(19) United States
(12) Patent Application Publication
(10) Pub. No.: US 2025/0361322 A1
(43) Pub. Date: Nov. 27, 2025

(54) ANTI-TISSUE FACTOR ANTIBODIES, ANTIBODY-DRUG CONJUGATES, AND RELATED METHODS

(71) Applicant: ICONIC THERAPEUTICS LLC, Palo Alto, CA (US)

(72) Inventors: Jan-Willem THEUNISSEN, South San Francisco, CA (US); Andrew D. AVERY, II, South San Francisco, CA (US); Allen G. CAI, South San Francisco, CA (US); Anthony Byron COOPER, South San Francisco, CA (US); Thi-Sau MIGONE, South San Francisco, CA (US)

(73) Assignee: ICONIC THERAPEUTICS LLC, Palo Alto, CA (US)

(21) Appl. No.: 19/000,442

(22) Filed: Dec. 23, 2024

Related U.S. Application Data
(62) Division of application No. 17/877,853, filed on Jul. 29, 2022, now Pat. No. 12,215,167.
Application No. 17/877,853 is a division of application No. 17/458,507, filed on Aug. 26, 2021, now Pat. No. 11,447,566.
Application No. 17/458,507 is a continuation of application No. 16/959,652, filed on Jul. 1, 2020, now abandoned.
(60) Provisional application No. 62/613,545, filed on Jan. 4, 2018.
(60) Provisional application No. 62/613,564, filed on Jan. 4, 2018.
(60) Provisional application No. 62/646,788, filed on Mar. 22, 2018.
(60) Provisional application No. 62/713,797, filed on Aug. 2, 2018.
(60) Provisional application No. 62/713,804, filed on Aug. 2, 2018.

(51) Int. Cl. C07K 16/36; A61K 31/713; A61K 47/68; A61P 27/02; C12N 5/10; C12N 15/63

(52) U.S. Cl. CPC C07K 16/36; A61K 31/713; A61K 47/68031; A61K 47/6843; A61K 47/6849; A61P 27/02; C12N 5/10; C12N 15/63; C07K 2317/21; C07K 2317/24; C07K 2317/33; C07K 2317/34; C07K 2317/56; C07K 2317/565; C07K 2317/76; C07K 2317/92

(57) ABSTRACT

Provided herein are antibodies that specifically bind to human tissue factor (TF), anti-TF antibody-drug conjugates (ADCs), and compositions comprising the antibodies or ADCs. Also provided herein are methods of making and using the antibodies or ADCs, such as therapeutic and diagnostic methods.

### CROSS REFERENCE TO RELATED APPLICATIONS

This application is a divisional of U.S. application Ser. No. 17/877,853, filed Jul. 29, 2022, which is a divisional of U.S. application Ser. No. 17/458,507, filed Aug. 26, 2021, now U.S. Pat. No. 11,447,566, which is a continuation of U.S. application Ser. No. 16/959,652, now abandoned, which is a U.S. National Stage Application under 35 U.S.C. § 371 of International Patent Application No. PCT/US2019/012427, filed Jan. 4, 2019, which claims the benefit of U.S. Provisional Application No. 62/713,804, filed Aug. 2, 2018, U.S. Provisional Application No. 62/713,797, filed Aug. 2, 2018, U.S. Provisional Application No. 62/646,788, filed Mar. 22, 2018, U.S. Provisional Application No. 62/613,564, filed Jan. 4, 2018 and U.S. Provisional Application No. 62/613,545, filed Jan. 4, 2018, the disclosure of each of which is incorporated by reference herein in its entirety.

### SEQUENCE LISTING

This application contains an electronic Sequence Listing which has been submitted in XML file format via Patent Center, the entire content of which is incorporated by reference herein in its entirety. The Sequence Listing XML file submitted via Patent Center is entitled “14529-140-999_SL.xml”, was created on Dec. 23, 2024, and is 823,022 bytes in size.

### BACKGROUND

Blood coagulation involves a complex set of processes that result in blood clotting. Tissue factor (TF) plays an important role in these coagulation processes. TF is a cell surface receptor for the serine protease factor VIIa (FVIIa). The TF/FVIIa complex catalyzes conversion of the inactive protease factor X (FX) into the active protease factor Xa (FXa). FXa and its co-factor FVa form the prothrombinase complex, which generates thrombin from prothrombin. Thrombin converts soluble fibrinogen into insoluble strands of fibrin and catalyzes many other coagulation-related processes.

TF is over-expressed on multiple types of solid tumors. In cancer, TF/FVIIa signaling can support angiogenesis, tumor progression, and metastasis. Increased TF expression can also induce inflammation and/or angiogenesis in many other diseases, including wet age-related macular degeneration (AMD) and diabetic retinopathy.

### SUMMARY

Provided herein are antibodies that specifically bind human Tissue Factor (TF), anti-TF antibody-drug conjugates, and related methods.

In one aspect, provided herein is an isolated human antibody which binds to the extracellular domain of human Tissue Factor (TF), wherein the antibody binds human TF at a human TF binding site that is distinct from a human TF binding site bound by human FVIIa.

In some embodiments, (1) the isolated antibody does not inhibit human thrombin generation as determined by thrombin generation assay (TGA) compared to a reference antibody comprising a V_{H} sequence of SEQ ID NO:821 and a V_{L} sequence of SEQ ID NO:822, and (2) the binding between the isolated antibody and a variant TF extracellular domain comprising a mutation at amino acid residue 149 of the sequence shown in SEQ ID NO:810 is less than 50% of the binding between the isolated antibody and the extracellular domain of TF of the sequence shown in SEQ ID NO:810, as determined by the median fluorescence intensity value of the isolated antibody relative to an isotype control in a live cell staining assay.

In some embodiments, (1) the isolated antibody inhibits human thrombin generation to a lesser extent as determined by thrombin generation assay (TGA) compared to a reference antibody comprising a V_{H} sequence of SEQ ID NO:821 and a V_{L} sequence of SEQ ID NO:822, and (2) the binding between the isolated antibody and a variant TF extracellular domain comprising a mutation at amino acid residue 149 of the sequence shown in SEQ ID NO:810 is less than 50% of the binding between the isolated antibody and the extracellular domain of TF of the sequence shown in SEQ ID NO:810, as determined by the median fluorescence intensity value of the isolated antibody relative to an isotype control in a live cell staining assay.

In some embodiments, (1) the isolated antibody allows human thrombin generation to a greater extent as determined by thrombin generation assay (TGA) compared to a reference antibody comprising a V_{H} sequence of SEQ ID NO:821 and a V_{L} sequence of SEQ ID NO:822, and (2) the binding between the isolated antibody and a variant TF extracellular domain comprising a mutation at amino acid residue 149 of the sequence shown in SEQ ID NO:810 is less than 50% of the binding between the isolated antibody and the extracellular domain of TF of the sequence shown in SEQ ID NO:810, as determined by the median fluorescence intensity value of the isolated antibody relative to an isotype control in a live cell staining assay.

In some embodiments, (1) the isolated antibody inhibits human thrombin generation by a lesser amount as determined by thrombin generation assay (TGA) compared to a reference antibody comprising a V_{H} sequence of SEQ ID NO:821 and a V_{L} sequence of SEQ ID NO:822, and (2) the binding between the isolated antibody and a variant TF extracellular domain comprising a mutation at amino acid residue 149 of the sequence shown in SEQ ID NO:810 is less than 50% of the binding between the isolated antibody and the extracellular domain of TF of the sequence shown in SEQ ID NO:810, as determined by the median fluorescence intensity value of the isolated antibody relative to an isotype control in a live cell staining assay.

In some embodiments, (1) the isolated antibody allows human thrombin generation by a greater amount as determined by thrombin generation assay (TGA) compared to a reference antibody comprising a V_{H} sequence of SEQ ID NO:821 and a V_{L} sequence of SEQ ID NO:822, and (2) the binding between the isolated antibody and a variant TF extracellular domain comprising a mutation at amino acid residue 149 of the sequence shown in SEQ ID NO:810 is less than 50% of the binding between the isolated antibody and the extracellular domain of TF of the sequence shown in SEQ ID NO:810, as determined by the median fluorescence intensity value of the isolated antibody relative to an isotype control in a live cell staining assay.

In some embodiments, the antibody comprises: a VH-CDR1 comprising the sequence set forth in SEQ ID NO:779; a VH-CDR2 comprising the sequence set forth in SEQ ID NO:780; a VH-CDR3 comprising the sequence set forth in SEQ ID NO:781; a VL-CDR1 comprising the sequence set forth in SEQ ID NO:782; a VL-CDR2 comprising the sequence set forth in SEQ ID NO:783; and a VL-CDR3 comprising the sequence set forth in SEQ ID NO:784.

In some embodiments, the antibody comprises: a VH-CDR1 comprising the sequence set forth in SEQ ID NO:872; a VH-CDR2 comprising the sequence set forth in SEQ ID NO:873; a VH-CDR3 comprising the sequence set forth in SEQ ID NO:874; a VL-CDR1 comprising the sequence set forth in SEQ ID NO:875; a VL-CDR2 comprising the sequence set forth in SEQ ID NO:876; and a VL-CDR3 comprising the sequence set forth in SEQ ID NO:877.

In some embodiments, the antibody comprises: a VH-CDR1 comprising the sequence set forth in SEQ ID NO:878; a VH-CDR2 comprising the sequence set forth in SEQ ID NO:879; a VH-CDR3 comprising the sequence set forth in SEQ ID NO:880; a VL-CDR1 comprising the sequence set forth in SEQ ID NO:881; a VL-CDR2 comprising the sequence set forth in SEQ ID NO:882; and a VL-CDR3 comprising the sequence set forth in SEQ ID NO:883.

In some embodiments, the isolated antibody does not inhibit human thrombin generation as determined by thrombin generation assay (TGA) compared to a reference antibody comprising a V_{H} sequence of SEQ ID NO:821 and a VL sequence of SEQ ID NO:822.

In some embodiments, the isolated antibody inhibits human thrombin generation to a lesser extent as determined by thrombin generation assay (TGA) compared to a reference antibody comprising a V_{H} sequence of SEQ ID NO:821 and a VL sequence of SEQ ID NO:822.

In some embodiments, the isolated antibody allows human thrombin generation to a greater extent as determined by thrombin generation assay (TGA) compared to a reference antibody comprising a V_{H} sequence of SEQ ID NO:821 and a VL sequence of SEQ ID NO:822.

In some embodiments, the isolated antibody inhibits human thrombin generation by a lesser amount as determined by thrombin generation assay (TGA) compared to a reference antibody comprising a V_{H} sequence of SEQ ID NO:821 and a VL sequence of SEQ ID NO:822.

In some embodiments, the isolated antibody allows human thrombin generation by a greater amount as determined by thrombin generation assay (TGA) compared to a reference antibody comprising a V_{H} sequence of SEQ ID NO:821 and a VL sequence of SEQ ID NO:822.

In some embodiments, the antibody does not inhibit human thrombin generation as determined by thrombin generation assay (TGA). In some embodiments, the antibody does not reduce the thrombin peak on a thrombin generation curve (Peak IIa) compared to an isotype control. In some embodiments, the antibody does not increase the time from the assay start to the thrombin peak on a thrombin generation curve (ttPeak) compared to an isotype control. In some embodiments, the antibody does not decrease the endogenous thrombin potential (ETP) as determined by the area under a thrombin generation curve compared to an isotype control. In some embodiments, the antibody allows human thrombin generation as determined by thrombin generation assay (TGA). In some embodiments, the antibody maintains the thrombin peak on a thrombin generation curve (Peak IIa) compared to an isotype control. In some embodiments, the antibody maintains the time from the assay start to the thrombin peak on a thrombin generation curve (ttPeak) compared to an isotype control. In some embodiments, the antibody preserves the endogenous thrombin potential (ETP) as determined by the area under a thrombin generation curve compared to an isotype control.

In some embodiments, the antibody binds human TF at a human TF binding site that is distinct from a human TF binding site bound by human FX. In some embodiments, the antibody does not interfere with the ability of TF:FVIIa to convert FX into FXa.

In some embodiments, the antibody does not compete for binding to human TF with human FVIIa.

In some embodiments, the antibody does not inhibit human thrombin generation as determined by thrombin generation assay (TGA), allows human thrombin generation as determined by thrombin generation assay (TGA), binds human TF at a human TF binding site that is distinct from a human TF binding site bound by human FX, does not interfere with the ability of TF:FVIIa to convert FX into FXa, and does not compete for binding to human TF with FVIIa.

In some embodiments, the antibody does not inhibit human thrombin generation as determined by thrombin generation assay (TGA), does not decrease the endogenous thrombin potential (ETP) as determined by the area under a thrombin generation curve compared to an isotype control, allows human thrombin generation as determined by thrombin generation assay (TGA), preserves the endogenous thrombin potential (ETP) as determined by the area under a thrombin generation curve compared to an isotype control, binds human TF at a human TF binding site that is distinct from a human TF binding site bound by human FX, does not interfere with the ability of TF:FVIIa to convert FX into FXa, and does not compete for binding to human TF with FVIIa.

In some embodiments, the antibody does not inhibit human thrombin generation as determined by thrombin generation assay (TGA), does not reduce the thrombin peak on a thrombin generation curve (Peak IIa) compared to an isotype control, does not increase the time from the assay start to the thrombin peak on a thrombin generation curve (ttPeak) compared to an isotype control, does not decrease the endogenous thrombin potential (ETP) as determined by the area under a thrombin generation curve compared to an isotype control, allows human thrombin generation as determined by thrombin generation assay (TGA), maintains the thrombin peak on a thrombin generation curve (Peak IIa) compared to an isotype control, maintains the time from the assay start to the thrombin peak on a thrombin generation curve (ttPeak) compared to an isotype control, preserves the endogenous thrombin potential (ETP) as determined by the area under a thrombin generation curve compared to an isotype control, binds human TF at a human TF binding site that is distinct from a human TF binding site bound by human FX, does not interfere with the ability of TF:FVIIa to convert FX into FXa, and does not compete for binding to human TF with FVIIa.

In some embodiments, the antibody inhibits FVIIa-dependent TF signaling.

In some embodiments, the binding between the isolated antibody and a variant TF extracellular domain comprising a mutation at amino acid residue 149 of the sequence shown in SEQ ID NO:810 is less than 50% of the binding between the isolated antibody and the extracellular domain of TF of the sequence shown in SEQ ID NO:810, as determined by the median fluorescence intensity value of the isolated antibody relative to an isotype control in a live cell staining assay. In some embodiments, the mutation at amino acid residue 149 of the sequence shown in SEQ ID NO:810 is K149N.

In some embodiments, the binding between the isolated antibody and a variant TF extracellular domain comprising a mutation at amino acid residue 68 of the sequence shown in SEQ ID NO:810 is greater than 50% of the binding between the isolated antibody and the extracellular domain of TF of the sequence shown in SEQ ID NO:810, as determined by the median fluorescence intensity value of the isolated antibody relative to an isotype control in a live cell staining assay. In some embodiments, the mutation at amino acid residue 68 of the sequence shown in SEQ ID NO:810 is K68N.

In some embodiments, the binding between the isolated antibody and a variant TF extracellular domain comprising mutations at amino acid residues 171 and 197 of the sequence shown in SEQ ID NO:810 is less than 50% of the binding between the isolated antibody and the extracellular domain of TF of the sequence shown in SEQ ID NO:810, as determined by the median fluorescence intensity value of the isolated antibody relative to an isotype control in a live cell staining assay. In some embodiments, the mutations at amino acid residues 171 and 197 of the sequence shown in SEQ ID NO:810 are N171H and T197K.

In some embodiments, the binding between the isolated antibody and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the isolated antibody and the extracellular domain of TF of the sequence shown in SEQ ID NO:810, as determined by the median fluorescence intensity value of the isolated antibody relative to an isotype control in a live cell staining assay.

In some embodiments, the binding between the isolated antibody and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the isolated antibody and the extracellular domain of TF of the sequence shown in SEQ ID NO:810, as determined by the median fluorescence intensity value of the isolated antibody relative to an isotype control in a live cell staining assay.

In some embodiments, the binding between the isolated antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the isolated antibody and the extracellular domain of TF of the sequence shown in SEQ ID NO:810, as determined by the median fluorescence intensity value of the isolated antibody relative to an isotype control in a live cell staining assay.

In some embodiments, the binding between the isolated antibody and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the isolated antibody and the extracellular domain of TF of the sequence shown in SEQ ID NO:810, as determined by the median fluorescence intensity value of the isolated antibody relative to an isotype control in a live cell staining assay.

In some embodiments, the binding between the isolated antibody and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the isolated antibody and the extracellular domain of TF of the sequence shown in SEQ ID NO:810, as determined by the median fluorescence intensity value of the isolated antibody relative to an isotype control in a live cell staining assay.

In some embodiments, the binding between the isolated antibody and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the isolated antibody and the extracellular domain of TF of the sequence shown in SEQ ID NO:810, as determined by the median fluorescence intensity value of the isolated antibody relative to an isotype control in a live cell staining assay.

In some embodiments, the binding between the isolated antibody and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the isolated antibody and the extracellular domain of TF of the sequence shown in SEQ ID NO:810, as determined by the median fluorescence intensity value of the isolated antibody relative to an isotype control in a live cell staining assay.

In some embodiments, the binding between the isolated antibody and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the isolated antibody and the extracellular domain of TF of the sequence shown in SEQ ID NO:810, as determined by the median fluorescence intensity value of the isolated antibody relative to an isotype control in a live cell staining assay.

In some embodiments, the binding between the isolated antibody and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the isolated antibody and the extracellular domain of TF of the sequence shown in SEQ ID NO:810, as determined by the median fluorescence intensity value of the isolated antibody relative to an isotype control in a live cell staining assay.

In some embodiments, the binding between the isolated antibody and a variant TF extracellular domain comprising a mutation at amino acid residue 149 of the sequence shown in SEQ ID NO:810 is less than 50% of the binding between the isolated antibody and the extracellular domain of TF of the sequence shown in SEQ ID NO:810; the binding between the isolated antibody and a variant TF extracellular domain comprising a mutation at amino acid residue 68 of the sequence shown in SEQ ID NO:810 is greater than 50% of the binding between the isolated antibody and the extracellular domain of TF of the sequence shown in SEQ ID NO:810; the binding between the isolated antibody and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the isolated antibody and the extracellular domain of TF of the sequence shown in SEQ ID NO:810; the binding between the isolated antibody and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the isolated antibody and the extracellular domain of TF of the sequence shown in SEQ ID NO:810; the binding between the isolated antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the isolated antibody and the extracellular domain of TF of the sequence shown in SEQ ID NO:810; the binding between the isolated antibody and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the isolated antibody and the extracellular domain of TF of the sequence shown in SEQ ID NO:810; and the binding between the isolated antibody and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the isolated antibody and the extracellular domain of TF of the sequence shown in SEQ ID NO:810, as determined by the median fluorescence intensity value of the antibody relative to an isotype control in a live cell staining assay. In some embodiments, the mutation at amino acid residue 149 of the sequence shown in SEQ ID NO:810 is K149N; and the mutation at amino acid residue 68 of the sequence shown in SEQ ID NO:810 is K68N.

In some embodiments, the binding between the isolated antibody and a variant TF extracellular domain comprising a mutation at amino acid residue 149 of the sequence shown in SEQ ID NO:810 is less than 50% of the binding between the isolated antibody and the extracellular domain of TF of the sequence shown in SEQ ID NO:810; the binding between the isolated antibody and a variant TF extracellular domain comprising a mutation at amino acid residue 68 of the sequence shown in SEQ ID NO:810 is greater than 50% of the binding between the isolated antibody and the extracellular domain of TF of the sequence shown in SEQ ID NO:810; the binding between the isolated antibody and a variant TF extracellular domain comprising mutations at amino acid residues 171 and 197 of the sequence shown in SEQ ID NO:810 is less than 50% of the binding between the isolated antibody and the extracellular domain of TF of the sequence shown in SEQ ID NO:810; the binding between the isolated antibody and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the isolated antibody and the extracellular domain of TF of the sequence shown in SEQ ID NO:810; the binding between the isolated antibody and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the isolated antibody and the extracellular domain of TF of the sequence shown in SEQ ID NO:810; the binding between the isolated antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the isolated antibody and the extracellular domain of TF of the sequence shown in SEQ ID NO:810; the binding between the isolated antibody and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the isolated antibody and the extracellular domain of TF of the sequence shown in SEQ ID NO:810; the binding between the isolated antibody and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the isolated antibody and the extracellular domain of TF of the sequence shown in SEQ ID NO:810; the binding between the isolated antibody and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the isolated antibody and the extracellular domain of TF of the sequence shown in SEQ ID NO:810; the binding between the isolated antibody and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the isolated antibody and the extracellular domain of TF of the sequence shown in SEQ ID NO:810; the binding between the isolated antibody and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the isolated antibody and the extracellular domain of TF of the sequence shown in SEQ ID NO:810; and the binding between the isolated antibody and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the isolated antibody and the extracellular domain of TF of the sequence shown in SEQ ID NO:810, as determined by the median fluorescence intensity value of the antibody relative to an isotype control in a live cell staining assay. In some embodiments, the mutation at amino acid residue 149 of the sequence shown in SEQ ID NO:810 is K149N; the mutation at amino acid residue 68 of the sequence shown in SEQ ID NO:810 is K68N; and the mutations at amino acid residues 171 and 197 of the sequence shown in SEQ ID NO:810 are N171H and T197K.

In some embodiments, the antibody binds to cynomolgus TF. In some embodiments, the antibody binds to mouse TF. In some embodiments, the antibody binds to rabbit TF. In some embodiments, the antibody binds to pig TF.

In some embodiments, the antibody reduces lesion size in a swine choroidal neovascularization (CNV) model.

In some embodiments, the antibody: (a) does not inhibit human thrombin generation as determined by thrombin generation assay (TGA); and (b) the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay. In some embodiments, the mutations at amino acid residues 171 and 197 of the sequence shown in SEQ ID NO:810 are N171H and T197K.

In some embodiments, the antibody: (a) allows human thrombin generation as determined by thrombin generation assay (TGA); and (b) the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay. In some embodiments, the mutations at amino acid residues 171 and 197 of the sequence shown in SEQ ID NO:810 are N171H and T197K.

In some embodiments, the antibody: (a) does not inhibit human thrombin generation as determined by thrombin generation assay (TGA); (b) the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and (c) the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay. In some embodiments, the mutation at amino acid residue 149 of the sequence shown in SEQ ID NO:810 is K149N; and the mutations at amino acid residues 171 and 197 of the sequence shown in SEQ ID NO:810 are N171H and T197K.

In some embodiments, the antibody: (a) allows human thrombin generation as determined by thrombin generation assay (TGA); (b) the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and (c) the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay. In some embodiments, the mutation at amino acid residue 149 of the sequence shown in SEQ ID NO:810 is K149N; and the mutations at amino acid residues 171 and 197 of the sequence shown in SEQ ID NO:810 are N171H and T197K.

In some embodiments, the antibody: (a) does not inhibit human thrombin generation as determined by thrombin generation assay (TGA); (b) binds to cynomolgus TF; (c) the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and (d) the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay. In some embodiments, the mutation at amino acid residue 149 of the sequence shown in SEQ ID NO:810 is K149N; and the mutations at amino acid residues 171 and 197 of the sequence shown in SEQ ID NO:810 are N171H and T197K.

In some embodiments, the antibody: (a) allows human thrombin generation as determined by thrombin generation assay (TGA); (b) binds to cynomolgus TF; (c) the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and (d) the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay. In some embodiments, the mutation at amino acid residue 149 of the sequence shown in SEQ ID NO:810 is K149N; and the mutations at amino acid residues 171 and 197 of the sequence shown in SEQ ID NO:810 are N171H and T197K.

In some embodiments, the antibody: (a) does not inhibit human thrombin generation as determined by thrombin generation assay (TGA); (b) allows human thrombin generation as determined by thrombin generation assay (TGA); (c) binds human TF at a human TF binding site that is distinct from a human TF binding site bound by human FX; (d) does not interfere with the ability of TF:FVIIa to convert FX into FXa; (e) does not compete for binding to human TF with FVIIa; (f) inhibits FVIIa-dependent TF signaling; (g) binds to cynomolgus TF; (h) binds to mouse TF; and (i) binds to rabbit TF.

In some embodiments, the antibody: (a) does not inhibit human thrombin generation as determined by thrombin generation assay (TGA); (b) does not decrease the endogenous thrombin potential (ETP) as determined by the area under a thrombin generation curve compared to an isotype control; (c) allows human thrombin generation as determined by thrombin generation assay (TGA); (d) preserves the endogenous thrombin potential (ETP) as determined by the area under a thrombin generation curve compared to an isotype control; (e) binds human TF at a human TF binding site that is distinct from a human TF binding site bound by human FX; (f) does not interfere with the ability of TF:FVIIa to convert FX into FXa; (g) does not compete for binding to human TF with FVIIa; (h) inhibits FVIIa-dependent TF signaling; (i) binds to cynomolgus TF; (j) binds to mouse TF; and (k) binds to rabbit TF.

In some embodiments, the antibody: (a) does not inhibit human thrombin generation as determined by thrombin generation assay (TGA); (b) does not reduce the thrombin peak on a thrombin generation curve (Peak IIa) compared to an isotype control; (c) does not increase the time from the assay start to the thrombin peak on a thrombin generation curve (ttPeak) compared to an isotype control; (d) does not decrease the endogenous thrombin potential (ETP) as determined by the area under a thrombin generation curve compared to an isotype control; (e) allows human thrombin generation as determined by thrombin generation assay (TGA); (f) maintains the thrombin peak on a thrombin generation curve (Peak IIa) compared to an isotype control; (g) maintains the time from the assay start to the thrombin peak on a thrombin generation curve (ttPeak) compared to an isotype control; (h) preserves the endogenous thrombin potential (ETP) as determined by the area under a thrombin generation curve compared to an isotype control; (i) binds human TF at a human TF binding site that is distinct from a human TF binding site bound by human FX; (j) does not interfere with the ability of TF:FVIIa to convert FX into FXa; (k) does not compete for binding to human TF with FVIIa; (l) inhibits FVIIa-dependent TF signaling; (m) binds to cynomolgus TF; (n) binds to mouse TF; and (o) binds to rabbit TF.

In some embodiments, the antibody: (a) does not inhibit human thrombin generation as determined by thrombin generation assay (TGA); (b) allows human thrombin generation as determined by thrombin generation assay (TGA); (c) binds human TF at a human TF binding site that is distinct from a human TF binding site bound by human FX; (d) does not interfere with the ability of TF:FVIIa to convert FX into FXa; (e) does not compete for binding to human TF with FVIIa; (f) inhibits FVIIa-dependent TF signaling; (g) binds to cynomolgus TF; (h) binds to mouse TF; (i) binds to rabbit TF; (j) binds to pig TF; and (k) reduces lesion size in a swine choroidal neovascularization (CNV) model.

In some embodiments, the antibody: (a) does not inhibit human thrombin generation as determined by thrombin generation assay (TGA); (b) does not decrease the endogenous thrombin potential (ETP) as determined by the area under a thrombin generation curve compared to an isotype control; (c) allows human thrombin generation as determined by thrombin generation assay (TGA); (d) preserves the endogenous thrombin potential (ETP) as determined by the area under a thrombin generation curve compared to an isotype control; (e) binds human TF at a human TF binding site that is distinct from a human TF binding site bound by human FX; (f) does not interfere with the ability of TF:FVIIa to convert FX into FXa; (g) does not compete for binding to human TF with FVIIa; (h) inhibits FVIIa-dependent TF signaling; (i) binds to cynomolgus TF; (j) binds to mouse TF; (k) binds to rabbit TF; (l) binds to pig TF; and (m) reduces lesion size in a swine choroidal neovascularization (CNV) model.

In some embodiments, the antibody: (a) does not inhibit human thrombin generation as determined by thrombin generation assay (TGA); (b) does not reduce the thrombin peak on a thrombin generation curve (Peak IIa) compared to an isotype control; (c) does not increase the time from the assay start to the thrombin peak on a thrombin generation curve (ttPeak) compared to an isotype control; (d) does not decrease the endogenous thrombin potential (ETP) as determined by the area under a thrombin generation curve compared to an isotype control; (e) allows human thrombin generation as determined by thrombin generation assay (TGA); (f) maintains the thrombin peak on a thrombin generation curve (Peak IIa) compared to an isotype control; (g) maintains the time from the assay start to the thrombin peak on a thrombin generation curve (ttPeak) compared to an isotype control; (h) preserves the endogenous thrombin potential (ETP) as determined by the area under a thrombin generation curve compared to an isotype control; (i) binds human TF at a human TF binding site that is distinct from a human TF binding site bound by human FX; (j) does not interfere with the ability of TF:FVIIa to convert FX into FXa; (k) does not compete for binding to human TF with FVIIa; (l) inhibits FVIIa-dependent TF signaling; (m) binds to cynomolgus TF; (n) binds to mouse TF; (o) binds to rabbit TF; (p) binds to pig TF; and (q) reduces lesion size in a swine choroidal neovascularization (CNV) model.

In some embodiments, the antibody: (a) does not inhibit human thrombin generation as determined by thrombin generation assay (TGA); (b) does not reduce the thrombin peak on a thrombin generation curve (Peak IIa) compared to an isotype control; (c) does not increase the time from the assay start to the thrombin peak on a thrombin generation curve (ttPeak) compared to an isotype control; (d) does not decrease the endogenous thrombin potential (ETP) as determined by the area under a thrombin generation curve compared to an isotype control; (e) allows human thrombin generation as determined by thrombin generation assay (TGA); (f) maintains the thrombin peak on a thrombin generation curve (Peak IIa) compared to an isotype control; (g) maintains the time from the assay start to the thrombin peak on a thrombin generation curve (ttPeak) compared to an isotype control; (h) preserves the endogenous thrombin potential (ETP) as determined by the area under a thrombin generation curve compared to an isotype control; (i) binds human TF at a human TF binding site that is distinct from a human TF binding site bound by human FX; (j) does not interfere with the ability of TF:FVIIa to convert FX into FXa; (k) does not compete for binding to human TF with FVIIa; (l) inhibits FVIIa-dependent TF signaling; (m) binds to cynomolgus TF; (n) binds to mouse TF; (o) binds to rabbit TF; (p) binds to pig TF; (q) reduces lesion size in a swine choroidal neovascularization (CNV) model; (r) the binding between the isolated antibody and a variant TF extracellular domain comprising a mutation at amino acid residue 149 of the sequence shown in SEQ ID NO:810 is less than 50% of the binding between the isolated antibody and the extracellular domain of TF of the sequence shown in SEQ ID NO:810, as determined by the median fluorescence intensity value of the isolated antibody relative to an isotype control in a live cell staining assay; (s) the binding between the isolated antibody and a variant TF extracellular domain comprising a mutation at amino acid residue 68 of the sequence shown in SEQ ID NO:810 is greater than 50% of the binding between the isolated antibody and the extracellular domain of TF of the sequence shown in SEQ ID NO:810, as determined by the median fluorescence intensity value of the isolated antibody relative to an isotype control in a live cell staining assay; (t) the binding between the isolated antibody and a variant TF extracellular domain comprising mutations at amino acid residues 171 and 197 of the sequence shown in SEQ ID NO:810 is less than 50% of the binding between the isolated antibody and the extracellular domain of TF of the sequence shown in SEQ ID NO:810, as determined by the median fluorescence intensity value of the isolated antibody relative to an isotype control in a live cell staining assay; (u) the binding between the isolated antibody and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the isolated antibody and the extracellular domain of TF of the sequence shown in SEQ ID NO:810, as determined by the median fluorescence intensity value of the isolated antibody relative to an isotype control in a live cell staining assay; (v) the binding between the isolated antibody and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the isolated antibody and the extracellular domain of TF of the sequence shown in SEQ ID NO:810, as determined by the median fluorescence intensity value of the isolated antibody relative to an isotype control in a live cell staining assay; (w) the binding between the isolated antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the isolated antibody and the extracellular domain of TF of the sequence shown in SEQ ID NO:810, as determined by the median fluorescence intensity value of the isolated antibody relative to an isotype control in a live cell staining assay; (x) the binding between the isolated antibody and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the isolated antibody and the extracellular domain of TF of the sequence shown in SEQ ID NO:810, as determined by the median fluorescence intensity value of the isolated antibody relative to an isotype control in a live cell staining assay; (y) the binding between the isolated antibody and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the isolated antibody and the extracellular domain of TF of the sequence shown in SEQ ID NO:810, as determined by the median fluorescence intensity value of the isolated antibody relative to an isotype control in a live cell staining assay; (z) the binding between the isolated antibody and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the isolated antibody and the extracellular domain of TF of the sequence shown in SEQ ID NO:810, as determined by the median fluorescence intensity value of the isolated antibody relative to an isotype control in a live cell staining assay; (aa) the binding between the isolated antibody and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the isolated antibody and the extracellular domain of TF of the sequence shown in SEQ ID NO:810, as determined by the median fluorescence intensity value of the isolated antibody relative to an isotype control in a live cell staining assay; (bb) the binding between the isolated antibody and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the isolated antibody and the extracellular domain of TF of the sequence shown in SEQ ID NO:810, as determined by the median fluorescence intensity value of the isolated antibody relative to an isotype control in a live cell staining assay; and (cc) wherein the binding between the isolated antibody and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the isolated antibody and the extracellular domain of TF of the sequence shown in SEQ ID NO:810, as determined by the median fluorescence intensity value of the isolated antibody relative to an isotype control in a live cell staining assay.

In some embodiments, the antibody: (a) does not inhibit human thrombin generation as determined by thrombin generation assay (TGA); (b) does not reduce the thrombin peak on a thrombin generation curve (Peak IIa) compared to an isotype control; (c) does not increase the time from the assay start to the thrombin peak on a thrombin generation curve (ttPeak) compared to an isotype control; (d) does not decrease the endogenous thrombin potential (ETP) as determined by the area under a thrombin generation curve compared to an isotype control; (e) allows human thrombin generation as determined by thrombin generation assay (TGA); (f) maintains the thrombin peak on a thrombin generation curve (Peak IIa) compared to an isotype control; (g) maintains the time from the assay start to the thrombin peak on a thrombin generation curve (ttPeak) compared to an isotype control; (h) preserves the endogenous thrombin potential (ETP) as determined by the area under a thrombin generation curve compared to an isotype control; (i) binds human TF at a human TF binding site that is distinct from a human TF binding site bound by human FX; (j) does not interfere with the ability of TF:FVIIa to convert FX into FXa; (k) does not compete for binding to human TF with FVIIa; (1) inhibits FVIIa-dependent TF signaling; (m) binds to cynomolgus TF; (n) binds to mouse TF; (o) binds to rabbit TF; (p) binds to pig TF; (q) reduces lesion size in a swine choroidal neovascularization (CNV) model; (r) the binding between the isolated antibody and a variant TF extracellular domain comprising a mutation K149N of the sequence shown in SEQ ID NO:810 is less than 50% of the binding between the isolated antibody and the extracellular domain of TF of the sequence shown in SEQ ID NO:810, as determined by the median fluorescence intensity value of the isolated antibody relative to an isotype control in a live cell staining assay; (s) the binding between the isolated antibody and a variant TF extracellular domain comprising a mutation K68N of the sequence shown in SEQ ID NO:810 is greater than 50% of the binding between the isolated antibody and the extracellular domain of TF of the sequence shown in SEQ ID NO:810, as determined by the median fluorescence intensity value of the isolated antibody relative to an isotype control in a live cell staining assay; (t) the binding between the isolated antibody and a variant TF extracellular domain comprising mutations N171H and T197K of the sequence shown in SEQ ID NO:810 is less than 50% of the binding between the isolated antibody and the extracellular domain of TF of the sequence shown in SEQ ID NO:810, as determined by the median fluorescence intensity value of the isolated antibody relative to an isotype control in a live cell staining assay; (u) the binding between the isolated antibody and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the isolated antibody and the extracellular domain of TF of the sequence shown in SEQ ID NO:810, as determined by the median fluorescence intensity value of the isolated antibody relative to an isotype control in a live cell staining assay; (v) the binding between the isolated antibody and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the isolated antibody and the extracellular domain of TF of the sequence shown in SEQ ID NO:810, as determined by the median fluorescence intensity value of the isolated antibody relative to an isotype control in a live cell staining assay; (w) the binding between the isolated antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the isolated antibody and the extracellular domain of TF of the sequence shown in SEQ ID NO:810, as determined by the median fluorescence intensity value of the isolated antibody relative to an isotype control in a live cell staining assay; (x) the binding between the isolated antibody and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the isolated antibody and the extracellular domain of TF of the sequence shown in SEQ ID NO:810, as determined by the median fluorescence intensity value of the isolated antibody relative to an isotype control in a live cell staining assay; (y) the binding between the isolated antibody and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the isolated antibody and the extracellular domain of TF of the sequence shown in SEQ ID NO:810, as determined by the median fluorescence intensity value of the isolated antibody relative to an isotype control in a live cell staining assay; (z) the binding between the isolated antibody and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the isolated antibody and the extracellular domain of TF of the sequence shown in SEQ ID NO:810, as determined by the median fluorescence intensity value of the isolated antibody relative to an isotype control in a live cell staining assay; (aa) the binding between the isolated antibody and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the isolated antibody and the extracellular domain of TF of the sequence shown in SEQ ID NO:810, as determined by the median fluorescence intensity value of the isolated antibody relative to an isotype control in a live cell staining assay; (bb) the binding between the isolated antibody and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the isolated antibody and the extracellular domain of TF of the sequence shown in SEQ ID NO:810, as determined by the median fluorescence intensity value of the isolated antibody relative to an isotype control in a live cell staining assay; and (cc) wherein the binding between the isolated antibody and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the isolated antibody and the extracellular domain of TF of the sequence shown in SEQ ID NO:810, as determined by the median fluorescence intensity value of the isolated antibody relative to an isotype control in a live cell staining assay.

In some embodiments, the antibody competes for binding to human TF with the antibody designated 25A, the antibody designated 25A3, the antibody designated 25A5, the antibody designated 25A5-T, the antibody designated 25G, the antibody designated 25G1, the antibody designated 25G9, the antibody designated 43B, the antibody designated 43B1, the antibody designated 43B7, the antibody designated 43D, the antibody designated 43D7, the antibody designated 43D8, the antibody designated 43E, or the antibody designated 43Ea.

In some embodiments, the antibody competes for binding to human TF with the antibody designated 25A, the antibody designated 25A3, the antibody designated 25A5, the antibody designated 25A5-T, the antibody designated 25G, the antibody designated 25G1, or the antibody designated 25G9.

In some embodiments, the antibody competes for binding to human TF with the antibody designated 43B, the antibody designated 43B1, the antibody designated 43B7, the antibody designated 43D, the antibody designated 43D7, the antibody designated 43D8, the antibody designated 43E, or the antibody designated 43Ea.

In some embodiments, the antibody binds to the same human TF epitope bound by the antibody designated 25A, the antibody designated 25A3, the antibody designated 25A5, the antibody designated 25A5-T, the antibody designated 25G, the antibody designated 25G1, the antibody designated 25G9, the antibody designated 43B, the antibody designated 43B1, the antibody designated 43B7, the antibody designated 43D, the antibody designated 43D7, the antibody designated 43D8, the antibody designated 43E, or the antibody designated 43Ea.

In some embodiments, the antibody binds to the same human TF epitope bound by the antibody designated 25A, the antibody designated 25A3, the antibody designated 25A5, the antibody designated 25A5-T, the antibody designated 25G, the antibody designated 25G1, or the antibody designated 25G9.

In some embodiments, the antibody binds to the same human TF epitope bound by the antibody designated 43B, the antibody designated 43B1, the antibody designated 43B7, the antibody designated 43D, the antibody designated 43D7, the antibody designated 43D8, the antibody designated 43E, or the antibody designated 43Ea.

In some embodiments, the antibody comprises all three heavy chain Complementary Determining Regions (CDRs) and all three light chain CDRs from: the antibody designated 25A, the antibody designated 25A3, the antibody designated 25A5, the antibody designated 25A5-T, the antibody designated 25G, the antibody designated 25G1, the antibody designated 25G9, the antibody designated 43B, the antibody designated 43B1, the antibody designated 43B7, the antibody designated 43D, the antibody designated 43D7, the antibody designated 43D8, the antibody designated 43E, or the antibody designated 43Ea. In some embodiments, the three heavy chain CDRs and the three light chain CDRs are determined using Kabat, Chothia, AbM, Contact, or IMGT numbering.

In some embodiments, the antibody comprises all three heavy chain Complementary Determining Regions (CDRs) and all three light chain CDRs from: the antibody designated 25A, the antibody designated 25A5-T, the antibody designated 25A3, the antibody designated 25A5, the antibody designated 25G, the antibody designated 25G1, or the antibody designated 25G9.

In some embodiments, the antibody comprises all three heavy chain Complementary Determining Regions (CDRs) and all three light chain CDRs from: the antibody designated 43B, the antibody designated 43B1, the antibody designated 43B7, the antibody designated 43D, the antibody designated 43D7, the antibody designated 43D8, the antibody designated 43E, or the antibody designated 43Ea.

In some embodiments, the antibody comprises all three heavy chain CDRs and all three light chain CDRs from the antibody designated 25A. In some embodiments, the antibody comprises all three heavy chain CDRs and all three light chain CDRs from the antibody designated 25A3. In some embodiments, the antibody comprises all three heavy chain CDRs and all three light chain CDRs from the antibody designated 25A5. In some embodiments, the antibody comprises all three heavy chain CDRs and all three light chain CDRs from the antibody designated 25A5-T. In some embodiments, the antibody comprises all three heavy chain CDRs and all three light chain CDRs from the antibody designated 25G.

In some embodiments, the antibody comprises all three heavy chain CDRs and all three light chain CDRs from the antibody designated 25G1. In some embodiments, the antibody comprises all three heavy chain CDRs and all three light chain CDRs from the antibody designated 25G9. In some embodiments, the antibody comprises all three heavy chain CDRs and all three light chain CDRs from the antibody designated 43B. In some embodiments, the antibody comprises all three heavy chain CDRs and all three light chain CDRs from the antibody designated 43B1. In some embodiments, the antibody comprises all three heavy chain CDRs and all three light chain CDRs from the antibody designated 43B7. In some embodiments, the antibody comprises all three heavy chain CDRs and all three light chain CDRs from the antibody designated 43D. In some embodiments, the antibody comprises all three heavy chain CDRs and all three light chain CDRs from the antibody designated 43D7.

In some embodiments, the antibody comprises all three heavy chain CDRs and all three light chain CDRs from the antibody designated 43D8. In some embodiments, the antibody comprises all three heavy chain CDRs and all three light chain CDRs from the antibody designated 43E. In some embodiments, the antibody comprises all three heavy chain CDRs and all three light chain CDRs from the antibody designated 43Ea.

In some embodiments, the antibody comprises a V_{H} sequence of SEQ ID NO: 113 and a V_{L} sequence of SEQ ID NO:114. In some embodiments, the antibody comprises a V_{H} sequence of SEQ ID NO:151 and a V_{L} sequence of SEQ ID NO:152. In some embodiments, the antibody comprises a V_{H} sequence of SEQ ID NO:189 and a V_{L} sequence of SEQ ID NO:190. In some embodiments, the antibody comprises a V_{H} sequence of SEQ ID NO:836 and a V_{L} sequence of SEQ ID NO:837. In some embodiments, the antibody comprises a V_{H} sequence of SEQ ID NO:227 and a V_{L} sequence of SEQ ID NO:228. In some embodiments, the antibody comprises a V_{H} sequence of SEQ ID NO:265 and a V_{L} sequence of SEQ ID NO:266. In some embodiments, the antibody comprises a V_{H} sequence of SEQ ID NO:303 and a V_{L} sequence of SEQ ID NO:304. In some embodiments, the antibody comprises a V_{H} sequence of SEQ ID NO:455 and a V_{L} sequence of SEQ ID NO:456. In some embodiments, the antibody comprises a V_{H} sequence of SEQ ID NO:493 and a V_{L} sequence of SEQ ID NO:494. In some embodiments, the antibody comprises a V_{H} sequence of SEQ ID NO:531 and a V_{L} sequence of SEQ ID NO:532. In some embodiments, the antibody comprises a V_{H} sequence of SEQ ID NO:569 and a V_{L} sequence of SEQ ID NO:570. In some embodiments, the antibody comprises a V_{H} sequence of SEQ ID NO:607 and a V_{L} sequence of SEQ ID NO:608. In some embodiments, the antibody comprises a V_{H} sequence of SEQ ID NO:645 and a V_{L} sequence of SEQ ID NO:646. In some embodiments, the antibody comprises a V_{H} sequence of SEQ ID NO:683 and a V_{L} sequence of SEQ ID NO:684. In some embodiments, the antibody comprises a V_{H} sequence of SEQ ID NO:721 and a V_{L} sequence of SEQ ID NO:722.

In some embodiments, the antibody comprises: a VH-CDR1 comprising the sequence set forth in SEQ ID NO:779; a VH-CDR2 comprising the sequence set forth in SEQ ID NO:780; a VH-CDR3 comprising the sequence set forth in SEQ ID NO:781; a VL-CDR1 comprising the sequence set forth in SEQ ID NO:782; a VL-CDR2 comprising the sequence set forth in SEQ ID NO:783; and a VL-CDR3 comprising the sequence set forth in SEQ ID NO:784. In some embodiments, the antibody comprises: a VH-CDR1 comprising the sequence set forth in SEQ ID NO:872; a VH-CDR2 comprising the sequence set forth in SEQ ID NO:873; a VH-CDR3 comprising the sequence set forth in SEQ ID NO:874; a VL-CDR1 comprising the sequence set forth in SEQ ID NO:875; a VL-CDR2 comprising the sequence set forth in SEQ ID NO:876; and a VL-CDR3 comprising the sequence set forth in SEQ ID NO:877. In some embodiments, the antibody comprises: a VH-CDR1 comprising the sequence set forth in SEQ ID NO:878; a VH-CDR2 comprising the sequence set forth in SEQ ID NO:879; a VH-CDR3 comprising the sequence set forth in SEQ ID NO:880; a VL-CDR1 comprising the sequence set forth in SEQ ID NO:881; a VL-CDR2 comprising the sequence set forth in SEQ ID NO:882; and a VL-CDR3 comprising the sequence set forth in SEQ ID NO:883. In some embodiments, the antibody comprises: a VH-CDR1 comprising the sequence set forth in SEQ ID NO:797; a VH-CDR2 comprising the sequence set forth in SEQ ID NO:798; a VH-CDR3 comprising the sequence set forth in SEQ ID NO:799; a VL-CDR1 comprising the sequence set forth in SEQ ID NO:800; a VL-CDR2 comprising the sequence set forth in SEQ ID NO:801; and a VL-CDR3 comprising the sequence set forth in SEQ ID NO:802.

In some embodiments, the antibody comprises a V_{H} sequence of SEQ ID NO:763 and a VL sequence of SEQ ID NO:764. In some embodiments, the antibody comprises a V_{H} sequence of SEQ ID NO:868 and a VL sequence of SEQ ID NO:869. In some embodiments, the antibody comprises a V_{H} sequence of SEQ ID NO:870 and a VL sequence of SEQ ID NO:871. In some embodiments, the antibody comprises a V_{H} sequence of SEQ ID NO:769 and a VL sequence of SEQ ID NO:770.

In some embodiments, the antibody comprises: the antibody designated 25A, the antibody designated 25A3, the antibody designated 25A5, the antibody designated 25A5-T, the antibody designated 25G, the antibody designated 25G1, the antibody designated 25G9, the antibody designated 43B, the antibody designated 43B1, the antibody designated 43B7, the antibody designated 43D, the antibody designated 43D7, the antibody designated 43D8, the antibody designated 43E, or the antibody designated 43Ea.

In some embodiments, the antibody comprises: the antibody designated 25A, the antibody designated 25A3, the antibody designated 25A5, the antibody designated 25A5-T, the antibody designated 25G, the antibody designated 25G1, or the antibody designated 25G9.

In some embodiments, the antibody comprises: the antibody designated 43B, the antibody designated 43B1, the antibody designated 43B7, the antibody designated 43D, the antibody designated 43D7, the antibody designated 43D8, the antibody designated 43E, or the antibody designated 43Ea.

In some embodiments, the antibody consists of: the antibody designated 25A, the antibody designated 25A3, the antibody designated 25A5, the antibody designated 25A5-T, the antibody designated 25G, the antibody designated 25G1, the antibody designated 25G9, the antibody designated 43B, the antibody designated 43B1, the antibody designated 43B7, the antibody designated 43D, the antibody designated 43D7, the antibody designated 43D8, the antibody designated 43E, or the antibody designated 43Ea.

In some embodiments, the antibody consists of: the antibody designated 25A, the antibody designated 25A3, the antibody designated 25A5, the antibody designated 25A5-T, the antibody designated 25G, the antibody designated 25G1, or the antibody designated 25G9.

In some embodiments, the antibody consists of: the antibody designated 43B, the antibody designated 43B1, the antibody designated 43B7, the antibody designated 43D, the antibody designated 43D7, the antibody designated 43D8, the antibody designated 43E, or the antibody designated 43Ea.

In another aspect, provided herein is an isolated antibody comprising all three heavy chain Complementary Determining Regions (CDRs) and all three light chain CDRs from: the antibody designated 25A, the antibody designated 25A3, the antibody designated 25A5, the antibody designated 25A5-T, the antibody designated 25G, the antibody designated 25G1, the antibody designated 25G9, the antibody designated 43B, the antibody designated 43B1, the antibody designated 43B7, the antibody designated 43D, the antibody designated 43D7, the antibody designated 43D8, the antibody designated 43E, or the antibody designated 43Ea.

In some embodiments, the antibody is human, humanized, or chimeric.

In some embodiments, the three heavy chain CDRs and the three light chain CDRs are determined using Kabat, Chothia, AbM, Contact, or IMGT numbering.

In some embodiments, the antibody comprises all three heavy chain CDRs and all three light chain CDRs from: the antibody designated 25A, the antibody designated 25A3, the antibody designated 25A5, the antibody designated 25A5-T, the antibody designated 25G, the antibody designated 25G1, or the antibody designated 25G9.

In some embodiments, the antibody comprises all three heavy chain CDRs and all three light chain CDRs from the antibody designated 25A. In some embodiments, the antibody comprises all three heavy chain CDRs and all three light chain CDRs from the antibody designated 25A3. In some embodiments, the antibody comprises all three heavy chain CDRs and all three light chain CDRs from the antibody designated 25A5. In some embodiments, the antibody comprises all three heavy chain CDRs and all three light chain CDRs from the antibody designated 25A5-T. In some embodiments, the antibody comprises all three heavy chain CDRs and all three light chain CDRs from the antibody designated 25G.

In some embodiments, the antibody comprises all three heavy chain CDRs and all three light chain CDRs from the antibody designated 25G1. In some embodiments, the antibody comprises all three heavy chain CDRs and all three light chain CDRs from the antibody designated 25G9.

In some embodiments, the antibody comprises all three heavy chain CDRs and all three light chain CDRs from: the antibody designated 43B, the antibody designated 43B1, the antibody designated 43B7, the antibody designated 43D, the antibody designated 43D7, the antibody designated 43D8, the antibody designated 43E, or the antibody designated 43Ea.

In some embodiments, the antibody comprises all three heavy chain CDRs and all three light chain CDRs from the antibody designated 43B. In some embodiments, the antibody comprises all three heavy chain CDRs and all three light chain CDRs from the antibody designated 43B1. In some embodiments, the antibody comprises all three heavy chain CDRs and all three light chain CDRs from the antibody designated 43B7. In some embodiments, the antibody comprises all three heavy chain CDRs and all three light chain CDRs from the antibody designated 43D. In some embodiments, the antibody comprises all three heavy chain CDRs and all three light chain CDRs from the antibody designated 43D7. In some embodiments, the antibody comprises all three heavy chain CDRs and all three light chain CDRs from the antibody designated 43D8. In some embodiments, the antibody comprises all three heavy chain CDRs and all three light chain CDRs from the antibody designated 43E. In some embodiments, the antibody comprises all three heavy chain CDRs and all three light chain CDRs from the antibody designated 43Ea.

In some embodiments, the antibody comprises a V_{H} sequence of SEQ ID NO: 113 and a V_{L} sequence of SEQ ID NO:114. In some embodiments, the antibody comprises a V_{H} sequence of SEQ ID NO:151 and a V_{L} sequence of SEQ ID NO:152. In some embodiments, the antibody comprises a V_{H} sequence of SEQ ID NO:189 and a V_{L} sequence of SEQ ID NO:190. In some embodiments, the antibody comprises a V_{H} sequence of SEQ ID NO:836 and a V_{L} sequence of SEQ ID NO:837. In some embodiments, the antibody comprises a V_{H} sequence of SEQ ID NO:227 and a V_{L} sequence of SEQ ID NO:228. In some embodiments, the antibody comprises a V_{H} sequence of SEQ ID NO:265 and a V_{L} sequence of SEQ ID NO:266. In some embodiments, the antibody comprises a V_{H} sequence of SEQ ID NO:303 and a V_{L} sequence of SEQ ID NO:304. In some embodiments, the antibody comprises a V_{H} sequence of SEQ ID NO:455 and a V_{L} sequence of SEQ ID NO:456. In some embodiments, the antibody comprises a V_{H} sequence of SEQ ID NO:493 and a V_{L} sequence of SEQ ID NO:494. In some embodiments, the antibody comprises a V_{H} sequence of SEQ ID NO:531 and a V_{L} sequence of SEQ ID NO:532. In some embodiments, the antibody comprises a V_{H} sequence of SEQ ID NO:569 and a V_{L} sequence of SEQ ID NO:570. In some embodiments, the antibody comprises a V_{H} sequence of SEQ ID NO:607 and a V_{L} sequence of SEQ ID NO:608. In some embodiments, the antibody comprises a V_{H} sequence of SEQ ID NO:645 and a V_{L} sequence of SEQ ID NO:646. In some embodiments, the antibody comprises a V_{H} sequence of SEQ ID NO:683 and a V_{L} sequence of SEQ ID NO:684. In some embodiments, the antibody comprises a V_{H} sequence of SEQ ID NO:721 and a V_{L} sequence of SEQ ID NO:722.

In some embodiments, the antibody comprises: a VH-CDR1 comprising the sequence set forth in SEQ ID NO:779; a VH-CDR2 comprising the sequence set forth in SEQ ID NO:780; a VH-CDR3 comprising the sequence set forth in SEQ ID NO:781; a VL-CDR1 comprising the sequence set forth in SEQ ID NO:782; a VL-CDR2 comprising the sequence set forth in SEQ ID NO:783; and a VL-CDR3 comprising the sequence set forth in SEQ ID NO:784. In some embodiments, the antibody comprises: a VH-CDR1 comprising the sequence set forth in SEQ ID NO:872; a VH-CDR2 comprising the sequence set forth in SEQ ID NO:873; a VH-CDR3 comprising the sequence set forth in SEQ ID NO:874; a VL-CDR1 comprising the sequence set forth in SEQ ID NO:875; a VL-CDR2 comprising the sequence set forth in SEQ ID NO:876; and a VL-CDR3 comprising the sequence set forth in SEQ ID NO:877. In some embodiments, the antibody comprises: a VH-CDR1 comprising the sequence set forth in SEQ ID NO:878; a VH-CDR2 comprising the sequence set forth in SEQ ID NO:879; a VH-CDR3 comprising the sequence set forth in SEQ ID NO:880; a VL-CDR1 comprising the sequence set forth in SEQ ID NO:881; a VL-CDR2 comprising the sequence set forth in SEQ ID NO:882; and a VL-CDR3 comprising the sequence set forth in SEQ ID NO:883. In some embodiments, the antibody comprises: a VH-CDR1 comprising the sequence set forth in SEQ ID NO:797; a VH-CDR2 comprising the sequence set forth in SEQ ID NO:798; a VH-CDR3 comprising the sequence set forth in SEQ ID NO:799; a VL-CDR1 comprising the sequence set forth in SEQ ID NO:800; a VL-CDR2 comprising the sequence set forth in SEQ ID NO:801; and a VL-CDR3 comprising the sequence set forth in SEQ ID NO:802.

In some embodiments, the antibody comprises a V_{H} sequence of SEQ ID NO:763 and a VL sequence of SEQ ID NO:764. In some embodiments, the antibody comprises a V_{H} sequence of SEQ ID NO:868 and a VL sequence of SEQ ID NO:869. In some embodiments, the antibody comprises a V_{H} sequence of SEQ ID NO:870 and a VL sequence of SEQ ID NO:871. In some embodiments, the antibody comprises a V_{H} sequence of SEQ ID NO:769 and a VL sequence of SEQ ID NO:770.

In some embodiments, the antibody comprises: the antibody designated 25A, the antibody designated 25A3, the antibody designated 25A5, the antibody designated 25A5-T, the antibody designated 25G, the antibody designated 25G1, the antibody designated 25G9, the antibody designated 43B, the antibody designated 43B1, the antibody designated 43B7, the antibody designated 43D, the antibody designated 43D7, the antibody designated 43D8, the antibody designated 43E, or the antibody designated 43Ea.

In some embodiments, the antibody comprises: the antibody designated 25A, the antibody designated 25A3, the antibody designated 25A5, the antibody designated 25A5-T, the antibody designated 25G, the antibody designated 25G1, or the antibody designated 25G9.

In some embodiments, the antibody comprises: the antibody designated 43B, the antibody designated 43B1, the antibody designated 43B7, the antibody designated 43D, the antibody designated 43D7, the antibody designated 43D8, the antibody designated 43E, or the antibody designated 43Ea.

In some embodiments, the antibody consists of: the antibody designated 25A, the antibody designated 25A3, the antibody designated 25A5, the antibody designated 25A5-T, the antibody designated 25G, the antibody designated 25G1, the antibody designated 25G9, the antibody designated 43B, the antibody designated 43B1, the antibody designated 43B7, the antibody designated 43D, the antibody designated 43D7, the antibody designated 43D8, the antibody designated 43E, or the antibody designated 43Ea.

In some embodiments, the antibody consists: the antibody designated 25A, the antibody designated 25A3, the antibody designated 25A5, the antibody designated 25A5-T, the antibody designated 25G, the antibody designated 25G1, or the antibody designated 25G9.

In some embodiments, the antibody consists: the antibody designated 43B, the antibody designated 43B1, the antibody designated 43B7, the antibody designated 43D, the antibody designated 43D7, the antibody designated 43D8, the antibody designated 43E, or the antibody designated 43Ea.

In another aspect, provided herein is an isolated antibody that competes for binding to human TF with: the antibody designated 1F, the antibody designated 1G, the antibody designated 29D, the antibody designated 29E, the antibody designated 39A, or the antibody designated 54E.

In some embodiments, the antibody is human, humanized, or chimeric.

In some embodiments, the antibody inhibits FVIIa-dependent TF signaling.

In some embodiments, the antibody binds to cynomolgus TF.

In some embodiments, the binding between the isolated antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the isolated antibody and the extracellular domain of TF of the sequence shown in SEQ ID NO:810, as determined by the median fluorescence intensity value of the isolated antibody relative to an isotype control in a live cell staining assay.

In some embodiments, the binding between the isolated antibody and a human TF extracellular domain with amino acid residues 78-93 of the sequence shown in SEQ ID NO:810 replaced by rat TF extracellular domain amino acid residues 77-98 of the sequence shown in SEQ ID NO:838 is less than 50% of the binding between the isolated antibody and the extracellular domain of TF of the sequence shown in SEQ ID NO:810, as determined by the median fluorescence intensity value of the isolated antibody relative to an isotype control in a live cell staining assay.

In some embodiments, the binding between the isolated antibody and a human TF extracellular domain with amino acid residues 78-107 of the sequence shown in SEQ ID NO:810 replaced by rat TF extracellular domain amino acid residues 77-112 of the sequence shown in SEQ ID NO:838 is greater than 50% of the binding between the isolated antibody and the extracellular domain of TF of the sequence shown in SEQ ID NO:810, as determined by the median fluorescence intensity value of the isolated antibody relative to an isotype control in a live cell staining assay.

In some embodiments, the binding between the isolated antibody and a human TF extracellular domain with amino acid residues 78-107 of the sequence shown in SEQ ID NO:810 replaced by rat TF extracellular domain amino acid residues 77-85 and 92-112 of the sequence shown in SEQ ID NO:838 is greater than 50% of the binding between the isolated antibody and the extracellular domain of TF of the sequence shown in SEQ ID NO:810, as determined by the median fluorescence intensity value of the isolated antibody relative to an isotype control in a live cell staining assay.

In some embodiments, the binding between the isolated antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the isolated antibody and the extracellular domain of TF of the sequence shown in SEQ ID NO:810; and the binding between the isolated antibody and a human TF extracellular domain with amino acid residues 78-93 of the sequence shown in SEQ ID NO:810 replaced by rat TF extracellular domain amino acid residues 77-98 of the sequence shown in SEQ ID NO:838 is less than 50% of the binding between the isolated antibody and the extracellular domain of TF of the sequence shown in SEQ ID NO:810, as determined by the median fluorescence intensity value of the isolated antibody relative to an isotype control in a live cell staining assay.

In some embodiments, the binding between the isolated antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the isolated antibody and the extracellular domain of TF of the sequence shown in SEQ ID NO:810; the binding between the isolated antibody and a human TF extracellular domain with amino acid residues 78-107 of the sequence shown in SEQ ID NO:810 replaced by rat TF extracellular domain amino acid residues 77-112 of the sequence shown in SEQ ID NO:838 is greater than 50% of the binding between the isolated antibody and the extracellular domain of TF of the sequence shown in SEQ ID NO:810; and wherein the binding between the isolated antibody and a human TF extracellular domain with amino acid residues 78-107 of the sequence shown in SEQ ID NO:810 replaced by rat TF extracellular domain amino acid residues 77-85 and 92-112 of the sequence shown in SEQ ID NO:838 is greater than 50% of the binding between the isolated antibody and the extracellular domain of TF of the sequence shown in SEQ ID NO:810, as determined by the median fluorescence intensity value of the isolated antibody relative to an isotype control in a live cell staining assay.

In some embodiments, the antibody comprises all three heavy chain Complementary Determining Regions (CDRs) and all three light chain CDRs from: the antibody designated 1F, the antibody designated 1G, the antibody designated 29D, the antibody designated 29E, the antibody designated 39A, the antibody designated 43Ea, or the antibody designated 54E. In some embodiments, the three heavy chain CDRs and the three light chain CDRs are determined using Kabat, Chothia, AbM, Contact, or IMGT numbering.

In some embodiments, the antibody comprises all three heavy chain CDRs and all three light chain CDRs from the antibody designated 1F. In some embodiments, the antibody comprises all three heavy chain CDRs and all three light chain CDRs from the antibody designated 1G. In some embodiments, the antibody comprises all three heavy chain CDRs and all three light chain CDRs from the antibody designated 29D. In some embodiments, the antibody comprises all three heavy chain CDRs and all three light chain CDRs from the antibody designated 29E. In some embodiments, the antibody comprises all three heavy chain CDRs and all three light chain CDRs from the antibody designated 39A. In some embodiments, the antibody comprises all three heavy chain CDRs and all three light chain CDRs from the antibody designated 54E.

In some embodiments, the antibody comprises a V_{H} sequence of SEQ ID NO:37 and a V_{L} sequence of SEQ ID NO:38. In some embodiments, the antibody comprises a V_{H} sequence of SEQ ID NO:75 and a V_{L} sequence of SEQ ID NO:76. In some embodiments, the antibody comprises a V_{H} sequence of SEQ ID NO:341 and a V_{L} sequence of SEQ ID NO:342. In some embodiments, the antibody comprises a V_{H} sequence of SEQ ID NO:379 and a V_{L} sequence of SEQ ID NO:380. In some embodiments, the antibody comprises a V_{H} sequence of SEQ ID NO:417 and a V_{L} sequence of SEQ ID NO:418. In some embodiments, the antibody comprises a V_{H} sequence of SEQ ID NO:759 and a V_{L} sequence of SEQ ID NO:760.

In some embodiments, the antibody comprises: a VH-CDR1 comprising the sequence set forth in SEQ ID NO:773; a VH-CDR2 comprising the sequence set forth in SEQ ID NO:774; a VH-CDR3 comprising the sequence set forth in SEQ ID NO:775; a VL-CDR1 comprising the sequence set forth in SEQ ID NO:776; a VL-CDR2 comprising the sequence set forth in SEQ ID NO:777; and a VL-CDR3 comprising the sequence set forth in SEQ ID NO:778. In some embodiments, the antibody comprises: a VH-CDR1 comprising the sequence set forth in SEQ ID NO:785; a VH-CDR2 comprising the sequence set forth in SEQ ID NO:786; a VH-CDR3 comprising the sequence set forth in SEQ ID NO:787; a VL-CDR1 comprising the sequence set forth in SEQ ID NO:788; a VL-CDR2 comprising the sequence set forth in SEQ ID NO:789; and a VL-CDR3 comprising the sequence set forth in SEQ ID NO:790. In some embodiments, the antibody comprises: a VH-CDR1 comprising the sequence set forth in SEQ ID NO:791; a VH-CDR2 comprising the sequence set forth in SEQ ID NO:792; a VH-CDR3 comprising the sequence set forth in SEQ ID NO:793; a VL-CDR1 comprising the sequence set forth in SEQ ID NO:794; a VL-CDR2 comprising the sequence set forth in SEQ ID NO:795; and a VL-CDR3 comprising the sequence set forth in SEQ ID NO:796. In some embodiments, the antibody comprises: a VH-CDR1 comprising the sequence set forth in SEQ ID NO:803; a VH-CDR2 comprising the sequence set forth in SEQ ID NO:804; a VH-CDR3 comprising the sequence set forth in SEQ ID NO:805; a VL-CDR1 comprising the sequence set forth in SEQ ID NO:806; a VL-CDR2 comprising the sequence set forth in SEQ ID NO:807; and a VL-CDR3 comprising the sequence set forth in SEQ ID NO:808.

In some embodiments, the antibody comprises a V_{H} sequence of SEQ ID NO:761 and a VL sequence of SEQ ID NO:762. In some embodiments, the antibody comprises a V_{H} sequence of SEQ ID NO:765 and a VL sequence of SEQ ID NO:766. In some embodiments, the antibody comprises a V_{H} sequence of SEQ ID NO:767 and a VL sequence of SEQ ID NO:768. In some embodiments, the antibody comprises a V_{H} sequence of SEQ ID NO:771 and a VL sequence of SEQ ID NO:772.

In some embodiments, the antibody comprises: the antibody designated 1F, the antibody designated 1G, the antibody designated 29D, the antibody designated 29E, the antibody designated 39A, or the antibody designated 54E.

In some embodiments, the antibody consists of: the antibody designated 1F, the antibody designated 1G, the antibody designated 29D, the antibody designated 29E, the antibody designated 39A, or the antibody designated 54E.

In another aspect, provided herein is an isolated antibody comprising: a VH-CDR1 comprising the sequence set forth in SEQ ID NO:773; a VH-CDR2 comprising the sequence set forth in SEQ ID NO:774; a VH-CDR3 comprising the sequence set forth in SEQ ID NO:775; a VL-CDR1 comprising the sequence set forth in SEQ ID NO:776; a VL-CDR2 comprising the sequence set forth in SEQ ID NO:777; and a VL-CDR3 comprising the sequence set forth in SEQ ID NO:778.

In another aspect, provided herein is an isolated antibody comprising: a VH-CDR1 comprising the sequence set forth in SEQ ID NO:779; a VH-CDR2 comprising the sequence set forth in SEQ ID NO:780; a VH-CDR3 comprising the sequence set forth in SEQ ID NO:781; a VL-CDR1 comprising the sequence set forth in SEQ ID NO:782; a VL-CDR2 comprising the sequence set forth in SEQ ID NO:783; and a VL-CDR3 comprising the sequence set forth in SEQ ID NO:784.

In another aspect, provided herein is an isolated antibody comprising: a VH-CDR1 comprising the sequence set forth in SEQ ID NO:785; a VH-CDR2 comprising the sequence set forth in SEQ ID NO:786; a VH-CDR3 comprising the sequence set forth in SEQ ID NO:787; a VL-CDR1 comprising the sequence set forth in SEQ ID NO:788; a VL-CDR2 comprising the sequence set forth in SEQ ID NO:789; and a VL-CDR3 comprising the sequence set forth in SEQ ID NO:790.

In another aspect, provided herein is an isolated antibody comprising: a VH-CDR1 comprising the sequence set forth in SEQ ID NO:791; a VH-CDR2 comprising the sequence set forth in SEQ ID NO:792; a VH-CDR3 comprising the sequence set forth in SEQ ID NO:793; a VL-CDR1 comprising the sequence set forth in SEQ ID NO:794; a VL-CDR2 comprising the sequence set forth in SEQ ID NO:795; and a VL-CDR3 comprising the sequence set forth in SEQ ID NO:796.

In another aspect, provided herein is an isolated antibody comprising: a VH-CDR1 comprising the sequence set forth in SEQ ID NO:797; a VH-CDR2 comprising the sequence set forth in SEQ ID NO:798; a VH-CDR3 comprising the sequence set forth in SEQ ID NO:799; a VL-CDR1 comprising the sequence set forth in SEQ ID NO:800; a VL-CDR2 comprising the sequence set forth in SEQ ID NO:801; and a VL-CDR3 comprising the sequence set forth in SEQ ID NO:802.

In another aspect, provided herein is an isolated antibody comprising: a VH-CDR1 comprising the sequence set forth in SEQ ID NO:803; a VH-CDR2 comprising the sequence set forth in SEQ ID NO:804; a VH-CDR3 comprising the sequence set forth in SEQ ID NO:805; a VL-CDR1 comprising the sequence set forth in SEQ ID NO:806; a VL-CDR2 comprising the sequence set forth in SEQ ID NO:807; and a VL-CDR3 comprising the sequence set forth in SEQ ID NO:808.

In another aspect, provided herein is an isolated antibody comprising: a VH-CDR1 comprising the sequence set forth in SEQ ID NO:872; a VH-CDR2 comprising the sequence set forth in SEQ ID NO:873; a VH-CDR3 comprising the sequence set forth in SEQ ID NO:874; a VL-CDR1 comprising the sequence set forth in SEQ ID NO:875; a VL-CDR2 comprising the sequence set forth in SEQ ID NO:876; and a VL-CDR3 comprising the sequence set forth in SEQ ID NO:877.

In another aspect, provided herein is an isolated antibody comprising: a VH-CDR1 comprising the sequence set forth in SEQ ID NO:878; a VH-CDR2 comprising the sequence set forth in SEQ ID NO:879; a VH-CDR3 comprising the sequence set forth in SEQ ID NO:880; a VL-CDR1 comprising the sequence set forth in SEQ ID NO:881; a VL-CDR2 comprising the sequence set forth in SEQ ID NO:882; and a VL-CDR3 comprising the sequence set forth in SEQ ID NO:883.

In some embodiments, the antibody binds to human TF with a KD of less than or equal to 50 nM, 10 nM, 5 nM, 1 nM, 0.5 nM or 0.1 nM, as measured by Octet QK384 or Biacore assay.

In some embodiments, the antibody is a monoclonal antibody.

In some embodiments, the antibody is multispecific.

In some embodiments, the antibody is a Fab, Fab′, F(ab′)2, Fv, scFv, (scFv)₂, single chain antibody molecule, dual variable domain antibody, single variable domain antibody, linear antibody, or V domain antibody.

In some embodiments, the antibody comprises a scaffold, optionally wherein the scaffold is Fc, optionally human Fc. In some embodiments, the antibody comprises a heavy chain constant region of a class selected from IgG, IgA, IgD, IgE, and IgM. In some embodiments, the antibody comprises a heavy chain constant region of the class IgG and a subclass selected from IgG1, IgG2, IgG3, and IgG4. In some embodiments, the antibody comprises a heavy chain constant region of IgG1.

In some embodiments, the Fc comprises one or more modifications, wherein the one or more modifications result in increased half-life, increased antibody-dependent cellular cytotoxicity (ADCC), increased antibody-dependent cellular phagocytosis (ADCP), increased complement-dependent cytotoxicity (CDC), or decreased effector function, compared with the Fc without the one or more modifications.

In another aspect, provided herein is an isolated antibody that competes for binding to human TF with any antibody above.

In another aspect, provided herein is an isolated antibody that binds the human TF epitope bound by any antibody above.

In another aspect, provided herein is an isolated polynucleotide or set of polynucleotides encoding any antibody above, a V_{H} thereof, a VL thereof, a light chain thereof, a heavy chain thereof, or an antigen-binding portion thereof.

In another aspect, provided herein is a vector or set of vectors comprising the polynucleotide or set of polynucleotides above.

In another aspect, provided herein is a host cell comprising the polynucleotide or set of polynucleotides above or the vector or set of vectors above.

In another aspect, provided herein is a method of producing an antibody comprising expressing the antibody with the host cell above and isolating the expressed antibody.

In another aspect, provided herein is a pharmaceutical composition comprising any antibody above and a pharmaceutically acceptable excipient.

In another aspect, provided herein is a method of treating or preventing a disease or condition in a subject in need thereof, comprising administering to the subject an effective amount of any antibody above or the pharmaceutical composition above.

In some embodiments, the disease or condition is cancer. In some embodiments, the cancer is head and neck cancer. In some embodiments, the cancer is ovarian cancer. In some embodiments, the cancer is gastric cancer. In some embodiments, the cancer is esophageal cancer. In some embodiments, the cancer is cervical cancer. In some embodiments, the cancer is prostate cancer. In some embodiments, the cancer is pancreatic cancer. In some embodiments, the cancer is estrogen receptors negative (ER−), progesterone receptors negative (PR−), and HER2 negative (HER2−) triple negative breast cancer. In some embodiments, the cancer is glioblastoma. In some embodiments, the cancer is lung cancer. In some embodiments, the cancer is bladder cancer. In some embodiments, the cancer is melanoma. In some embodiments, the cancer is kidney cancer.

In some embodiments, the disease or condition involves neovascularization. In some embodiments, the disease or condition involving neovascularization is age-related macular degeneration (AMD), diabetic retinopathy, or cancer. In some embodiments, the disease or condition involves vascular inflammation.

In some embodiments, the method further comprises administering one or more additional therapeutic agents to the subject. In some embodiments, the additional therapeutic agent is formulated in the same pharmaceutical composition as the antibody. In some embodiments, the additional therapeutic agent is formulated in a different pharmaceutical composition from the antibody. In some embodiments, the additional therapeutic agent is administered prior to administering the antibody. In some embodiments, the additional therapeutic agent is administered after administering the antibody. In some embodiments, the additional therapeutic agent is administered contemporaneously with the antibody.

In another aspect, provided herein is a method of detecting TF in a subject having or suspected of having a disease or condition, the method comprising: (a) receiving a sample from the subject; and (b) detecting the presence or the level of TF in the sample by contacting the sample with any antibody above.

In some embodiments, the disease or condition is cancer. In some embodiments, the cancer is head and neck cancer. In some embodiments, the cancer is ovarian cancer. In some embodiments, the cancer is gastric cancer. In some embodiments, the cancer is esophageal cancer. In some embodiments, the cancer is cervical cancer. In some embodiments, the cancer is prostate cancer. In some embodiments, the cancer is pancreatic cancer. In some embodiments, the cancer is estrogen receptors negative (ER−), progesterone receptors negative (PR−), and HER2 negative (HER2−) triple negative breast cancer. In some embodiments, the cancer is glioblastoma. In some embodiments, the cancer is lung cancer. In some embodiments, the cancer is bladder cancer. In some embodiments, the cancer is melanoma. In some embodiments, the cancer is kidney cancer.

In some embodiments, the disease or condition involves neovascularization. In some embodiments, the disease or condition involving neovascularization is age-related macular degeneration (AMD), diabetic retinopathy, or cancer. In some embodiments, the disease or condition involves vascular inflammation.

In another aspect, provided herein is a method of detecting TF in a subject having or suspected of having a disease or condition, the method comprising: (a) administering to the subject any antibody above; and (b) detecting the presence or the level of TF in the subject.

In some embodiments, the disease or condition is cancer. In some embodiments, the cancer is head and neck cancer. In some embodiments, the cancer is ovarian cancer. In some embodiments, the cancer is gastric cancer. In some embodiments, the cancer is esophageal cancer. In some embodiments, the cancer is cervical cancer. In some embodiments, the cancer is prostate cancer. In some embodiments, the cancer is pancreatic cancer. In some embodiments, the cancer is estrogen receptors negative (ER−), progesterone receptors negative (PR−), and HER2 negative (HER2−) triple negative breast cancer. In some embodiments, the cancer is glioblastoma. In some embodiments, the cancer is lung cancer. In some embodiments, the cancer is bladder cancer. In some embodiments, the cancer is melanoma. In some embodiments, the cancer is kidney cancer.

In some embodiments, the disease or condition involves neovascularization. In some embodiments, the disease or condition involving neovascularization is age-related macular degeneration (AMD), diabetic retinopathy, or cancer. In some embodiments, the disease or condition involves vascular inflammation.

In another aspect, provided herein is a kit comprising any antibody above or the pharmaceutical composition above and instructions for use.

In another aspect, provided herein is an antibody-drug conjugate comprising: an anti-human Tissue Factor (anti-hTF) antibody, a cytotoxic agent linked to the antibody, and optionally a linker that links the antibody to the cytotoxic agent, wherein the antibody binds to the extracellular domain of human Tissue Factor (TF) at a human TF binding site that is distinct from a human TF binding site bound by human FVIIa.

In some embodiments, (1) the antibody does not inhibit human thrombin generation as determined by thrombin generation assay (TGA) compared to a reference antibody comprising a V_{H} sequence of SEQ ID NO:821 and a V_{L} sequence of SEQ ID NO:822, and (2) the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, (1) the antibody inhibits human thrombin generation to a lesser extent as determined by thrombin generation assay (TGA) compared to a reference antibody comprising a V_{H} sequence of SEQ ID NO:821 and a V_{L} sequence of SEQ ID NO:822, and (2) the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, (1) the antibody allows human thrombin generation to a greater extent as determined by thrombin generation assay (TGA) compared to a reference antibody comprising a V_{H} sequence of SEQ ID NO:821 and a V_{L} sequence of SEQ ID NO:822, and (2) the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, (1) the antibody inhibits human thrombin generation by a lesser amount as determined by thrombin generation assay (TGA) compared to a reference antibody comprising a V_{H} sequence of SEQ ID NO:821 and a V_{L} sequence of SEQ ID NO:822, and (2) the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, (1) the antibody allows human thrombin generation by a greater amount as determined by thrombin generation assay (TGA) compared to a reference antibody comprising a V_{H} sequence of SEQ ID NO:821 and a V_{L} sequence of SEQ ID NO:822, and (2) the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the antibody comprises: a VH-CDR1 comprising the sequence set forth in SEQ ID NO:779; a VH-CDR2 comprising the sequence set forth in SEQ ID NO:780; a VH-CDR3 comprising the sequence set forth in SEQ ID NO:781; a VL-CDR1 comprising the sequence set forth in SEQ ID NO:782; a VL-CDR2 comprising the sequence set forth in SEQ ID NO:783; and a VL-CDR3 comprising the sequence set forth in SEQ ID NO:784.

In some embodiments, the antibody comprises: a VH-CDR1 comprising the sequence set forth in SEQ ID NO:872; a VH-CDR2 HI comprising the sequence set forth in SEQ ID NO:873; a VH-CDR3 comprising the sequence set forth in SEQ ID NO:874; a VL-CDR1 comprising the sequence set forth in SEQ ID NO:875; a VL-CDR2 comprising the sequence set forth in SEQ ID NO:876; and a VL-CDR3 comprising the sequence set forth in SEQ ID NO:877.

In some embodiments, the antibody comprises: a VH-CDR1 comprising the sequence set forth in SEQ ID NO:878; a VH-CDR2 comprising the sequence set forth in SEQ ID NO:879; a VH-CDR3 comprising the sequence set forth in SEQ ID NO:880; a VL-CDR1 comprising the sequence set forth in SEQ ID NO:881; a VL-CDR2 comprising the sequence set forth in SEQ ID NO:882; and a VL-CDR3 comprising the sequence set forth in SEQ ID NO:883.

In some embodiments, the antibody does not inhibit human thrombin generation as determined by thrombin generation assay (TGA) compared to a reference antibody comprising a V_{H} sequence of SEQ ID NO:821 and a VL sequence of SEQ ID NO:822.

In some embodiments, the antibody inhibits human thrombin generation to a lesser extent as determined by thrombin generation assay (TGA) compared to a reference antibody comprising a V_{H} sequence of SEQ ID NO:821 and a VL sequence of SEQ ID NO:822.

In some embodiments, the antibody allows human thrombin generation to a greater extent as determined by thrombin generation assay (TGA) compared to a reference antibody comprising a V_{H} sequence of SEQ ID NO:821 and a VL sequence of SEQ ID NO:822.

In some embodiments, the antibody inhibits human thrombin generation by a lesser amount as determined by thrombin generation assay (TGA) compared to a reference antibody comprising a V_{H} sequence of SEQ ID NO:821 and a VL sequence of SEQ ID NO:822.

In some embodiments, the antibody allows human thrombin generation by a greater amount as determined by thrombin generation assay (TGA) compared to a reference antibody comprising a V_{H} sequence of SEQ ID NO:821 and a VL sequence of SEQ ID NO:822.

In some embodiments, the antibody does not inhibit human thrombin generation as determined by thrombin generation assay (TGA). In some embodiments, the antibody does not reduce the thrombin peak on a thrombin generation curve (Peak IIa) compared to an isotype control. In some embodiments, the antibody does not increase the time from the assay start to the thrombin peak on a thrombin generation curve (ttPeak) compared to an isotype control. In some embodiments, the antibody does not decrease the endogenous thrombin potential (ETP) as determined by the area under a thrombin generation curve compared to an isotype control. In some embodiments, the antibody allows human thrombin generation as determined by thrombin generation assay (TGA). In some embodiments, the antibody maintains the thrombin peak on a thrombin generation curve (Peak IIa) compared to an isotype control. In some embodiments, the antibody maintains the time from the assay start to the thrombin peak on a thrombin generation curve (ttPeak) compared to an isotype control. In some embodiments, the antibody preserves the endogenous thrombin potential (ETP) as determined by the area under a thrombin generation curve compared to an isotype control.

In some embodiments, the antibody binds human TF at a human TF binding site that is distinct from a human TF binding site bound by human FX. In some embodiments, the antibody does not interfere with the ability of TF:FVIIa to convert FX into FXa.

In some embodiments, the antibody does not compete for binding to human TF with human FVIIa.

In some embodiments, the antibody does not inhibit human thrombin generation as determined by thrombin generation assay (TGA), allows human thrombin generation as determined by thrombin generation assay (TGA), binds human TF at a human TF binding site that is distinct from a human TF binding site bound by human FX, does not interfere with the ability of TF:FVIIa to convert FX into FXa, and does not compete for binding to human TF with FVIIa.

In some embodiments, the antibody does not inhibit human thrombin generation as determined by thrombin generation assay (TGA), does not decrease the endogenous thrombin potential (ETP) as determined by the area under a thrombin generation curve compared to an isotype control, allows human thrombin generation as determined by thrombin generation assay (TGA), preserves the endogenous thrombin potential (ETP) as determined by the area under a thrombin generation curve compared to an isotype control, binds human TF at a human TF binding site that is distinct from a human TF binding site bound by human FX, does not interfere with the ability of TF:FVIIa to convert FX into FXa, and does not compete for binding to human TF with FVIIa.

In some embodiments, the antibody does not inhibit human thrombin generation as determined by thrombin generation assay (TGA), does not reduce the thrombin peak on a thrombin generation curve (Peak IIa) compared to an isotype control, does not increase the time from the assay start to the thrombin peak on a thrombin generation curve (ttPeak) compared to an isotype control, does not decrease the endogenous thrombin potential (ETP) as determined by the area under a thrombin generation curve compared to an isotype control, allows human thrombin generation as determined by thrombin generation assay (TGA), maintains the thrombin peak on a thrombin generation curve (Peak IIa) compared to an isotype control, maintains the time from the assay start to the thrombin peak on a thrombin generation curve (ttPeak) compared to an isotype control, preserves the endogenous thrombin potential (ETP) as determined by the area under a thrombin generation curve compared to an isotype control, binds human TF at a human TF binding site that is distinct from a human TF binding site bound by human FX, does not interfere with the ability of TF:FVIIa to convert FX into FXa, and does not compete for binding to human TF with FVIIa.

In some embodiments, the antibody inhibits FVIIa-dependent TF signaling.

In some embodiments, the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay. In some embodiments, the mutation at amino acid residue 149 of the sequence shown in SEQ ID NO:810 is K149N.

In some embodiments, the binding between the antibody and a variant TF extracellular domain comprising a mutation at amino acid residue 68 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay. In some embodiments, the mutation at amino acid residue 68 of the sequence shown in SEQ ID NO:810 is K68N.

In some embodiments, the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay. In some embodiments, the mutations at amino acid residues 171 and 197 of the sequence shown in SEQ ID NO:810 are N171H and T197K.

In some embodiments, the binding between the antibody and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between the antibody and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between the antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between the antibody and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between the antibody and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between the antibody and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between the antibody and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between the antibody and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between the antibody and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, the binding between the antibody and a variant TF extracellular domain comprising a mutation at amino acid residue 68 of the sequence shown in SEQ ID NO:810 is greater than 50% of the binding between the antibody and the extracellular domain of TF of the sequence shown in SEQ ID NO:810, the binding between the antibody and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810, the binding between the antibody and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810, the binding between the antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810, the binding between the antibody and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810, the binding between the antibody and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay. In some embodiments, the mutation at amino acid residue 149 of the sequence shown in SEQ ID NO:810 is K149N; and the mutation at amino acid residue 68 of the sequence shown in SEQ ID NO:810 is K68N.

In some embodiments, the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, the binding between the antibody and a variant TF extracellular domain comprising a mutation at amino acid residue 68 of the sequence shown in SEQ ID NO:810 is greater than 50% of the binding between the antibody and the extracellular domain of TF of the sequence shown in SEQ ID NO:810, the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, the binding between the antibody and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810, the binding between the antibody and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810, the binding between the antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810, the binding between the antibody and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810, the binding between the antibody and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the antibody and the extracellular domain of TF of the sequence shown in SEQ ID NO:810, the binding between the antibody and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the antibody and the extracellular domain of TF of the sequence shown in SEQ ID NO:810, the binding between the antibody and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the antibody and the extracellular domain of TF of the sequence shown in SEQ ID NO:810, the binding between the antibody and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the antibody and the extracellular domain of TF of the sequence shown in SEQ ID NO:810, and wherein the binding between the antibody and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay. In some embodiments, the mutation at amino acid residue 149 of the sequence shown in SEQ ID NO:810 is K149N; the mutation at amino acid residue 68 of the sequence shown in SEQ ID NO:810 is K68N; and the mutations at amino acid residues 171 and 197 of the sequence shown in SEQ ID NO:810 are N171H and T197K.

In some embodiments, the antibody binds to cynomolgus TF. In some embodiments, the antibody binds to mouse TF. In some embodiments, the antibody binds to rabbit TF. In some embodiments, the antibody binds to pig TF.

In some embodiments, the antibody: (a) does not inhibit human thrombin generation as determined by thrombin generation assay (TGA); and (b) the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay. In some embodiments, the mutations at amino acid residues 171 and 197 of the sequence shown in SEQ ID NO:810 are N171H and T197K.

In some embodiments, the antibody: (a) allows human thrombin generation as determined by thrombin generation assay (TGA); and (b) the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay. In some embodiments, the mutations at amino acid residues 171 and 197 of the sequence shown in SEQ ID NO:810 are N171H and T197K.

In some embodiments, the antibody: (a) does not inhibit human thrombin generation as determined by thrombin generation assay (TGA); (b) the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and (c) the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay. In some embodiments, the mutation at amino acid residue 149 of the sequence shown in SEQ ID NO:810 is K149N; and the mutations at amino acid residues 171 and 197 of the sequence shown in SEQ ID NO:810 are N171H and T197K.

In some embodiments, the antibody: (a) allows human thrombin generation as determined by thrombin generation assay (TGA); (b) the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and (c) the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay. In some embodiments, the mutation at amino acid residue 149 of the sequence shown in SEQ ID NO:810 is K149N; and the mutations at amino acid residues 171 and 197 of the sequence shown in SEQ ID NO:810 are N171H and T197K.

In some embodiments, the antibody: (a) does not inhibit human thrombin generation as determined by thrombin generation assay (TGA); (b) binds to cynomolgus TF; (c) the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and (d) the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay. In some embodiments, the mutation at amino acid residue 149 of the sequence shown in SEQ ID NO:810 is K149N; and the mutations at amino acid residues 171 and 197 of the sequence shown in SEQ ID NO:810 are N171H and T197K.

In some embodiments, the antibody: (a) allows human thrombin generation as determined by thrombin generation assay (TGA); (b) binds to cynomolgus TF; (c) the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and (d) the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay. In some embodiments, the mutation at amino acid residue 149 of the sequence shown in SEQ ID NO:810 is K149N; and the mutations at amino acid residues 171 and 197 of the sequence shown in SEQ ID NO:810 are N171H and T197K.

In some embodiments, the antibody: (a) does not inhibit human thrombin generation as determined by thrombin generation assay (TGA); (b) allows human thrombin generation as determined by thrombin generation assay (TGA); (c) binds human TF at a human TF binding site that is distinct from a human TF binding site bound by human FX; (d) does not interfere with the ability of TF:FVIIa to convert FX into FXa; (e) does not compete for binding to human TF with FVIIa; (f) inhibits FVIIa-dependent TF signaling; (g) binds to cynomolgus TF; (h) binds to mouse TF; and (i) binds to rabbit TF.

In some embodiments, the antibody: (a) does not inhibit human thrombin generation as determined by thrombin generation assay (TGA); (b) does not decrease the endogenous thrombin potential (ETP) as determined by the area under a thrombin generation curve compared to an isotype control; (c) allows human thrombin generation as determined by thrombin generation assay (TGA); (d) preserves the endogenous thrombin potential (ETP) as determined by the area under a thrombin generation curve compared to an isotype control; (e) binds human TF at a human TF binding site that is distinct from a human TF binding site bound by human FX; (f) does not interfere with the ability of TF:FVIIa to convert FX into FXa; (g) does not compete for binding to human TF with FVIIa; (h) inhibits FVIIa-dependent TF signaling; (i) binds to cynomolgus TF; (j) binds to mouse TF; and (k) binds to rabbit TF.

In some embodiments, the antibody: (a) does not inhibit human thrombin generation as determined by thrombin generation assay (TGA); (b) does not reduce the thrombin peak on a thrombin generation curve (Peak IIa) compared to an isotype control; (c) does not increase the time from the assay start to the thrombin peak on a thrombin generation curve (ttPeak) compared to an isotype control; (d) does not decrease the endogenous thrombin potential (ETP) as determined by the area under a thrombin generation curve compared to an isotype control; (e) allows human thrombin generation as determined by thrombin generation assay (TGA); (f) maintains the thrombin peak on a thrombin generation curve (Peak IIa) compared to an isotype control; (g) maintains the time from the assay start to the thrombin peak on a thrombin generation curve (ttPeak) compared to an isotype control; (h) preserves the endogenous thrombin potential (ETP) as determined by the area under a thrombin generation curve compared to an isotype control; (i) binds human TF at a human TF binding site that is distinct from a human TF binding site bound by human FX; (j) does not interfere with the ability of TF:FVIIa to convert FX into FXa; (k) does not compete for binding to human TF with FVIIa; (l) inhibits FVIIa-dependent TF signaling; (m) binds to cynomolgus TF; (n) binds to mouse TF; and (o) binds to rabbit TF.

In some embodiments, the antibody: (a) does not inhibit human thrombin generation as determined by thrombin generation assay (TGA); (b) allows human thrombin generation as determined by thrombin generation assay (TGA); (c) binds human TF at a human TF binding site that is distinct from a human TF binding site bound by human FX; (d) does not interfere with the ability of TF:FVIIa to convert FX into FXa; (e) does not compete for binding to human TF with FVIIa; (f) inhibits FVIIa-dependent TF signaling; (g) binds to cynomolgus TF; (h) binds to mouse TF; (i) binds to rabbit TF; and (j) binds to pig TF.

In some embodiments, the antibody: (a) does not inhibit human thrombin generation as determined by thrombin generation assay (TGA); (b) does not decrease the endogenous thrombin potential (ETP) as determined by the area under a thrombin generation curve compared to an isotype control; (c) allows human thrombin generation as determined by thrombin generation assay (TGA); (d) preserves the endogenous thrombin potential (ETP) as determined by the area under a thrombin generation curve compared to an isotype control; (e) binds human TF at a human TF binding site that is distinct from a human TF binding site bound by human FX; (f) does not interfere with the ability of TF:FVIIa to convert FX into FXa; (g) does not compete for binding to human TF with FVIIa; (h) inhibits FVIIa-dependent TF signaling; (i) binds to cynomolgus TF; (j) binds to mouse TF; (k) binds to rabbit TF; and (l) binds to pig TF.

In some embodiments, the antibody: (a) does not inhibit human thrombin generation as determined by thrombin generation assay (TGA); (b) does not reduce the thrombin peak on a thrombin generation curve (Peak IIa) compared to an isotype control; (c) does not increase the time from the assay start to the thrombin peak on a thrombin generation curve (ttPeak) compared to an isotype control; (d) does not decrease the endogenous thrombin potential (ETP) as determined by the area under a thrombin generation curve compared to an isotype control; (e) allows human thrombin generation as determined by thrombin generation assay (TGA); (f) maintains the thrombin peak on a thrombin generation curve (Peak IIa) compared to an isotype control; (g) maintains the time from the assay start to the thrombin peak on a thrombin generation curve (ttPeak) compared to an isotype control; (h) preserves the endogenous thrombin potential (ETP) as determined by the area under a thrombin generation curve compared to an isotype control; (i) binds human TF at a human TF binding site that is distinct from a human TF binding site bound by human FX; (j) does not interfere with the ability of TF:FVIIa to convert FX into FXa; (k) does not compete for binding to human TF with FVIIa; (l) inhibits FVIIa-dependent TF signaling; (m) binds to cynomolgus TF; (n) binds to mouse TF; (o) binds to rabbit TF; and (p) binds to pig TF.

In some embodiments, the antibody: (a) does not inhibit human thrombin generation as determined by thrombin generation assay (TGA); (b) does not reduce the thrombin peak on a thrombin generation curve (Peak IIa) compared to an isotype control; (c) does not increase the time from the assay start to the thrombin peak on a thrombin generation curve (ttPeak) compared to an isotype control; (d) does not decrease the endogenous thrombin potential (ETP) as determined by the area under a thrombin generation curve compared to an isotype control; (e) allows human thrombin generation as determined by thrombin generation assay (TGA); (f) maintains the thrombin peak on a thrombin generation curve (Peak IIa) compared to an isotype control; (g) maintains the time from the assay start to the thrombin peak on a thrombin generation curve (ttPeak) compared to an isotype control; (h) preserves the endogenous thrombin potential (ETP) as determined by the area under a thrombin generation curve compared to an isotype control; (i) binds human TF at a human TF binding site that is distinct from a human TF binding site bound by human FX; (j) does not interfere with the ability of TF:FVIIa to convert FX into FXa; (k) does not compete for binding to human TF with FVIIa; (l) inhibits FVIIa-dependent TF signaling; (m) binds to cynomolgus TF; (n) binds to mouse TF; (o) binds to rabbit TF; (p) binds to pig TF; (q) the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (r) the binding between the antibody and a variant TF extracellular domain comprising a mutation at amino acid residue 68 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay; (s) the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (t) the binding between the antibody and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (u) the binding between the antibody and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (v) the binding between the antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (w) the binding between the antibody and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (x) the binding between the antibody and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (y) the binding between the antibody and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (z) the binding between the antibody and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (aa) the binding between the antibody and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; and (bb) the binding between the antibody and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the antibody: (a) does not inhibit human thrombin generation as determined by thrombin generation assay (TGA); (b) does not reduce the thrombin peak on a thrombin generation curve (Peak IIa) compared to an isotype control; (c) does not increase the time from the assay start to the thrombin peak on a thrombin generation curve (ttPeak) compared to an isotype control; (d) does not decrease the endogenous thrombin potential (ETP) as determined by the area under a thrombin generation curve compared to an isotype control; (e) allows human thrombin generation as determined by thrombin generation assay (TGA); (f) maintains the thrombin peak on a thrombin generation curve (Peak IIa) compared to an isotype control; (g) maintains the time from the assay start to the thrombin peak on a thrombin generation curve (ttPeak) compared to an isotype control; (h) preserves the endogenous thrombin potential (ETP) as determined by the area under a thrombin generation curve compared to an isotype control; (i) binds human TF at a human TF binding site that is distinct from a human TF binding site bound by human FX; (j) does not interfere with the ability of TF:FVIIa to convert FX into FXa; (k) does not compete for binding to human TF with FVIIa; (1) inhibits FVIIa-dependent TF signaling; (m) binds to cynomolgus TF; (n) binds to mouse TF; (o) binds to rabbit TF; (p) binds to pig TF; (q) the binding between the antibody and a variant TF extracellular domain comprising a mutation K149N of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (r) the binding between the antibody and a variant TF extracellular domain comprising a mutation K68N of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay; (s) the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (t) the binding between the antibody and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (u) the binding between the antibody and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (v) the binding between the antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (w) the binding between the antibody and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (x) the binding between the antibody and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (y) the binding between the antibody and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (z) the binding between the antibody and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (aa) the binding between the antibody and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; and (bb) the binding between the antibody and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the antibody competes for binding to human TF with the antibody designated 25A, the antibody designated 25A3, the antibody designated 25A5, the antibody designated 25A5-T, the antibody designated 25G, the antibody designated 25G1, the antibody designated 25G9, the antibody designated 43B, the antibody designated 43B1, the antibody designated 43B7, the antibody designated 43D, the antibody designated 43D7, the antibody designated 43D8, the antibody designated 43E, or the antibody designated 43Ea.

In some embodiments, the antibody competes for binding to human TF with the antibody designated 25A, the antibody designated 25A3, the antibody designated 25A5, the antibody designated 25A5-T, the antibody designated 25G, the antibody designated 25G1, or the antibody designated 25G9.

In some embodiments, the antibody competes for binding to human TF with the antibody designated 43B, the antibody designated 43B1, the antibody designated 43B7, the antibody designated 43D, the antibody designated 43D7, the antibody designated 43D8, the antibody designated 43E, or the antibody designated 43Ea.

In some embodiments, the antibody binds to the same human TF epitope bound by the antibody designated 25A, the antibody designated 25A3, the antibody designated 25A5, the antibody designated 25A5-T, the antibody designated 25G, the antibody designated 25G1, the antibody designated 25G9, the antibody designated 43B, the antibody designated 43B1, the antibody designated 43B7, the antibody designated 43D, the antibody designated 43D7, the antibody designated 43D8, the antibody designated 43E, or the antibody designated 43Ea.

In some embodiments, the antibody binds to the same human TF epitope bound by the antibody designated 25A, the antibody designated 25A3, the antibody designated 25A5, the antibody designated 25A5-T, the antibody designated 25G, the antibody designated 25G1, or the antibody designated 25G9.

In some embodiments, the antibody binds to the same human TF epitope bound by the antibody designated 43B, the antibody designated 43B1, the antibody designated 43B7, the antibody designated 43D, the antibody designated 43D7, the antibody designated 43D8, the antibody designated 43E, or the antibody designated 43Ea.

In some embodiments, the antibody comprises all three heavy chain Complementary Determining Regions (CDRs) and all three light chain CDRs from: the antibody designated 25A, the antibody designated 25A3, the antibody designated 25A5, the antibody designated 25A5-T, the antibody designated 25G, the antibody designated 25G1, the antibody designated 25G9, the antibody designated 43B, the antibody designated 43B1, the antibody designated 43B7, the antibody designated 43D, the antibody designated 43D7, the antibody designated 43D8, the antibody designated 43E, or the antibody designated 43Ea. In some embodiments, the three heavy chain CDRs and the three light chain CDRs are determined using Kabat, Chothia, AbM, Contact, or IMGT numbering.

In some embodiments, the antibody comprises all three heavy chain Complementary Determining Regions (CDRs) and all three light chain CDRs from: the antibody designated 25A, the antibody designated 25A3, the antibody designated 25A5, the antibody designated 25A5-T, the antibody designated 25G, the antibody designated 25G1, or the antibody designated 25G9.

In some embodiments, the antibody comprises all three heavy chain Complementary Determining Regions (CDRs) and all three light chain CDRs from: the antibody designated 43B, the antibody designated 43B1, the antibody designated 43B7, the antibody designated 43D, the antibody designated 43D7, the antibody designated 43D8, the antibody designated 43E, or the antibody designated 43Ea.

In some embodiments, the antibody comprises all three heavy chain CDRs and all three light chain CDRs from the antibody designated 25A. In some embodiments, the antibody comprises all three heavy chain CDRs and all three light chain CDRs from the antibody designated 25A3. In some embodiments, the antibody comprises all three heavy chain CDRs and all three light chain CDRs from the antibody designated 25A5. In some embodiments, the antibody comprises all three heavy chain CDRs and all three light chain CDRs from the antibody designated 25A5-T. In some embodiments, the antibody comprises all three heavy chain CDRs and all three light chain CDRs from the antibody designated 25G. In some embodiments, the antibody comprises all three heavy chain CDRs and all three light chain CDRs from the antibody designated 25G1. In some embodiments, the antibody comprises all three heavy chain CDRs and all three light chain CDRs from the antibody designated 25G9. In some embodiments, the antibody comprises all three heavy chain CDRs and all three light chain CDRs from the antibody designated 43B. In some embodiments, the antibody comprises all three heavy chain CDRs and all three light chain CDRs from the antibody designated 43B1. In some embodiments, the antibody comprises all three heavy chain CDRs and all three light chain CDRs from the antibody designated 43B7. In some embodiments, the antibody comprises all three heavy chain CDRs and all three light chain CDRs from the antibody designated 43D. In some embodiments, the antibody comprises all three heavy chain CDRs and all three light chain CDRs from the antibody designated 43D7. In some embodiments, the antibody comprises all three heavy chain CDRs and all three light chain CDRs from the antibody designated 43D8. In some embodiments, the antibody comprises all three heavy chain CDRs and all three light chain CDRs from the antibody designated 43E. In some embodiments, the antibody comprises all three heavy chain CDRs and all three light chain CDRs from the antibody designated 43Ea.

In some embodiments, the antibody comprises a V_{H} sequence of SEQ ID NO:113 and a V_{L} sequence of SEQ ID NO:114. In some embodiments, the antibody comprises a V_{H} sequence of SEQ ID NO:151 and a V_{L} sequence of SEQ ID NO:152. In some embodiments, the antibody comprises a V_{H} sequence of SEQ ID NO:189 and a V_{L} sequence of SEQ ID NO:190. In some embodiments, the antibody comprises a V_{H} sequence of SEQ ID NO:836 and a V_{L} sequence of SEQ ID NO:837. In some embodiments, the antibody comprises a V_{H} sequence of SEQ ID NO:227 and a V_{L} sequence of SEQ ID NO:228. In some embodiments, the antibody comprises a V_{H} sequence of SEQ ID NO:265 and a V_{L} sequence of SEQ ID NO:266. In some embodiments, the antibody comprises a V_{H} sequence of SEQ ID NO:303 and a VL sequence of SEQ ID NO:304. In some embodiments, the antibody comprises a V_{H} sequence of SEQ ID NO:455 and a VL sequence of SEQ ID NO:456. In some embodiments, the antibody comprises a V_{H} sequence of SEQ ID NO:493 and a VL sequence of SEQ ID NO:494. In some embodiments, the antibody comprises a V_{H} sequence of SEQ ID NO:531 and a VL sequence of SEQ ID NO:532. In some embodiments, the antibody comprises a V_{H} sequence of SEQ ID NO:569 and a VL sequence of SEQ ID NO:570. In some embodiments, the antibody comprises a V_{H} sequence of SEQ ID NO:607 and a VL sequence of SEQ ID NO:608. In some embodiments, the antibody comprises a V_{H} sequence of SEQ ID NO:645 and a VL sequence of SEQ ID NO:646. In some embodiments, the antibody comprises a V_{H} sequence of SEQ ID NO:683 and a VL sequence of SEQ ID NO:684. In some embodiments, the antibody comprises a V_{H} sequence of SEQ ID NO:721 and a VL sequence of SEQ ID NO:722.

In some embodiments, the antibody comprises: a VH-CDR1 comprising the sequence set forth in SEQ ID NO:779; a VH-CDR2 comprising the sequence set forth in SEQ ID NO:780; a VH-CDR3 comprising the sequence set forth in SEQ ID NO:781; a VL-CDR1 comprising the sequence set forth in SEQ ID NO:782; a VL-CDR2 comprising the sequence set forth in SEQ ID NO:783; and a VL-CDR3 comprising the sequence set forth in SEQ ID NO:784. In some embodiments, the antibody comprises: a VH-CDR1 comprising the sequence set forth in SEQ ID NO:872; a VH-CDR2 comprising the sequence set forth in SEQ ID NO:873; a VH-CDR3 comprising the sequence set forth in SEQ ID NO:874; a VL-CDR1 comprising the sequence set forth in SEQ ID NO:875; a VL-CDR2 comprising the sequence set forth in SEQ ID NO:876; and a VL-CDR3 comprising the sequence set forth in SEQ ID NO:877. In some embodiments, the antibody comprises: a VH-CDR1 comprising the sequence set forth in SEQ ID NO:878; a VH-CDR2 comprising the sequence set forth in SEQ ID NO:879; a VH-CDR3 comprising the sequence set forth in SEQ ID NO:880; a VL-CDR1 comprising the sequence set forth in SEQ ID NO:881; a VL-CDR2 comprising the sequence set forth in SEQ ID NO:882; and a VL-CDR3 comprising the sequence set forth in SEQ ID NO:883. In some embodiments, the antibody comprises: a VH-CDR1 comprising the sequence set forth in SEQ ID NO:797; a VH-CDR2 comprising the sequence set forth in SEQ ID NO:798; a VH-CDR3 comprising the sequence set forth in SEQ ID NO:799; a VL-CDR1 comprising the sequence set forth in SEQ ID NO:800; a VL-CDR2 comprising the sequence set forth in SEQ ID NO:801; and a VL-CDR3 comprising the sequence set forth in SEQ ID NO:802.

In some embodiments, the antibody comprises a V_{H} sequence of SEQ ID NO:763 and a V_{L} sequence of SEQ ID NO:764. In some embodiments, the antibody comprises a V_{H} sequence of SEQ ID NO:868 and a V_{L} sequence of SEQ ID NO:869. In some embodiments, the antibody comprises a V_{H} sequence of SEQ ID NO:870 and a V_{L} sequence of SEQ ID NO:871. In some embodiments, the antibody comprises a V_{H} sequence of SEQ ID NO:769 and a V_{L} sequence of SEQ ID NO:770.

In some embodiments, the antibody comprises: the antibody designated 25A, the antibody designated 25A3, the antibody designated 25A5, the antibody designated 25A5-T, the antibody designated 25G, the antibody designated 25G1, the antibody designated 25G9, the antibody designated 43B, the antibody designated 43B1, the antibody designated 43B7, the antibody designated 43D, the antibody designated 43D7, the antibody designated 43D8, the antibody designated 43E, or the antibody designated 43Ea. In some embodiments, the antibody comprises: the antibody designated 25A, the antibody designated 25A3, the antibody designated 25A5, the antibody designated 25A5-T, the antibody designated 25G, the antibody designated 25G1, or the antibody designated 25G9. In some embodiments, the antibody comprises: the antibody designated 43B, the antibody designated 43B1, the antibody designated 43B7, the antibody designated 43D, the antibody designated 43D7, the antibody designated 43D8, the antibody designated 43E, or the antibody designated 43Ea.

In some embodiments, the antibody consists of: the antibody designated 25A, the antibody designated 25A3, the antibody designated 25A5, the antibody designated 25A5-T, the antibody designated 25G, the antibody designated 25G1, the antibody designated 25G9, the antibody designated 43B, the antibody designated 43B1, the antibody designated 43B7, the antibody designated 43D, the antibody designated 43D7, the antibody designated 43D8, the antibody designated 43E, or the antibody designated 43Ea. In some embodiments, the antibody consists of: the antibody designated 25A, the antibody designated 25A3, the antibody designated 25A5, the antibody designated 25A5-T, the antibody designated 25G, the antibody designated 25G1, or the antibody designated 25G9. In some embodiments, the antibody consists of: the antibody designated 43B, the antibody designated 43B1, the antibody designated 43B7, the antibody designated 43D, the antibody designated 43D7, the antibody designated 43D8, the antibody designated 43E, or the antibody designated 43Ea.

In another aspect, provided herein is an antibody-drug conjugate comprising: an anti-human Tissue Factor (anti-hTF) antibody, a cytotoxic agent linked to the antibody, and optionally a linker that links the antibody to the cytotoxic agent, wherein the antibody competes for binding to human TF with: the antibody designated 1F, the antibody designated 1G, the antibody designated 29D, the antibody designated 29E, the antibody designated 39A, or the antibody designated 54E.

In some embodiments, the antibody inhibits FVIIa-dependent TF signaling.

In some embodiments, the antibody binds to cynomolgus TF.

In some embodiments, the binding between the antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between the antibody and a human TF extracellular domain with amino acid residues 78-93 of the sequence shown in SEQ ID NO:810 replaced by rat TF extracellular domain amino acid residues 77-98 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between the antibody and a human TF extracellular domain with amino acid residues 78-107 of the sequence shown in SEQ ID NO:810 replaced by rat TF extracellular domain amino acid residues 77-112 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between the antibody and a human TF extracellular domain with amino acid residues 78-107 of the sequence shown in SEQ ID NO:810 replaced by rat TF extracellular domain amino acid residues 77-85 and 92-112 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between the antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810; and the binding between the antibody and a human TF extracellular domain with amino acid residues 78-93 of the sequence shown in SEQ ID NO:810 replaced by rat TF extracellular domain amino acid residues 77-98 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between the antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810; the binding between the antibody and a human TF extracellular domain with amino acid residues 78-107 of the sequence shown in SEQ ID NO:810 replaced by rat TF extracellular domain amino acid residues 77-112 of the sequence shown in SEQ ID NO:838 is greater than 50% of the binding between the antibody and the extracellular domain of TF of the sequence shown in SEQ ID NO:810; and the binding between the antibody and a human TF extracellular domain with amino acid residues 78-107 of the sequence shown in SEQ ID NO:810 replaced by rat TF extracellular domain amino acid residues 77-85 and 92-112 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the antibody comprises all three heavy chain Complementary Determining Regions (CDRs) and all three light chain CDRs from: the antibody designated 1F, the antibody designated 1G, the antibody designated 29D, the antibody designated 29E, the antibody designated 39A, the antibody designated 43Ea, or the antibody designated 54E. In some embodiments, the three heavy chain CDRs and the three light chain CDRs are determined using Kabat, Chothia, AbM, Contact, or IMGT numbering.

In some embodiments, the antibody comprises all three heavy chain CDRs and all three light chain CDRs from the antibody designated 1F. In some embodiments, the antibody comprises all three heavy chain CDRs and all three light chain CDRs from the antibody designated 1G. In some embodiments, the antibody comprises all three heavy chain CDRs and all three light chain CDRs from the antibody designated 29D. In some embodiments, the antibody comprises all three heavy chain CDRs and all three light chain CDRs from the antibody designated 29E. In some embodiments, the antibody comprises all three heavy chain CDRs and all three light chain CDRs from the antibody designated 39A. In some embodiments, the antibody comprises all three heavy chain CDRs and all three light chain CDRs from the antibody designated 54E.

In some embodiments, the antibody comprises a V_{H} sequence of SEQ ID NO:37 and a V_{L} sequence of SEQ ID NO:38. In some embodiments, the antibody comprises a V_{H} sequence of SEQ ID NO:75 and a V_{L} sequence of SEQ ID NO:76. In some embodiments, the antibody comprises a V_{H} sequence of SEQ ID NO:341 and a V_{L} sequence of SEQ ID NO:342. In some embodiments, the antibody comprises a V_{H} sequence of SEQ ID NO:379 and a V_{L} sequence of SEQ ID NO:380. In some embodiments, the antibody comprises a V_{H} sequence of SEQ ID NO:417 and a V_{L} sequence of SEQ ID NO:418. In some embodiments, the antibody comprises a V_{H} sequence of SEQ ID NO:759 and a V_{L} sequence of SEQ ID NO:760.

In some embodiments, the antibody comprises: a VH-CDR1 comprising the sequence set forth in SEQ ID NO:773; a VH-CDR2 comprising the sequence set forth in SEQ ID NO:774; a VH-CDR3 comprising the sequence set forth in SEQ ID NO:775; a VL-CDR1 comprising the sequence set forth in SEQ ID NO:776; a VL-CDR2 comprising the sequence set forth in SEQ ID NO:777; and a VL-CDR3 comprising the sequence set forth in SEQ ID NO:778. In some embodiments, the antibody comprises: a VH-CDR1 comprising the sequence set forth in SEQ ID NO:785; a VH-CDR2 comprising the sequence set forth in SEQ ID NO:786; a VH-CDR3 comprising the sequence set forth in SEQ ID NO:787; a VL-CDR1 comprising the sequence set forth in SEQ ID NO:788; a VL-CDR2 comprising the sequence set forth in SEQ ID NO:789; and a VL-CDR3 comprising the sequence set forth in SEQ ID NO:790. In some embodiments, the antibody comprises: a VH-CDR1 comprising the sequence set forth in SEQ ID NO:791; a VH-CDR2 comprising the sequence set forth in SEQ ID NO:792; a VH-CDR3 comprising the sequence set forth in SEQ ID NO:793; a VL-CDR1 comprising the sequence set forth in SEQ ID NO:794; a VL-CDR2 comprising the sequence set forth in SEQ ID NO:795; and a VL-CDR3 comprising the sequence set forth in SEQ ID NO:796. In some embodiments, the antibody comprises: a VH-CDR1 comprising the sequence set forth in SEQ ID NO:803; a VH-CDR2 comprising the sequence set forth in SEQ ID NO:804; a VH-CDR3 comprising the sequence set forth in SEQ ID NO:805; a VL-CDR1 comprising the sequence set forth in SEQ ID NO:806; a VL-CDR2 comprising the sequence set forth in SEQ ID NO:807; and a VL-CDR3 comprising the sequence set forth in SEQ ID NO:808.

In some embodiments, the antibody comprises a V_{H} sequence of SEQ ID NO:761 and a V_{L} sequence of SEQ ID NO:762. In some embodiments, the antibody comprises a V_{H} sequence of SEQ ID NO:765 and a V_{L} sequence of SEQ ID NO:766. In some embodiments, the antibody comprises a V_{H} sequence of SEQ ID NO:767 and a V_{L} sequence of SEQ ID NO:768. In some embodiments, the antibody comprises a V_{H} sequence of SEQ ID NO:771 and a V_{L} sequence of SEQ ID NO:772.

In some embodiments, the antibody comprises: the antibody designated 1F, the antibody designated 1G, the antibody designated 29D, the antibody designated 29E, the antibody designated 39A, or the antibody designated 54E. In some embodiments, the antibody consists of: the antibody designated 1F, the antibody designated 1G, the antibody designated 29D, the antibody designated 29E, the antibody designated 39A, or the antibody designated 54E.

In another aspect, provided herein is an antibody-drug conjugate comprising: an anti-human Tissue Factor (anti-hTF) antibody, a cytotoxic agent linked to the antibody, and optionally a linker that links the antibody to the cytotoxic agent, wherein the antibody comprises: a VH-CDR1 comprising the sequence set forth in SEQ ID NO:773; a VH-CDR2 comprising the sequence set forth in SEQ ID NO:774; a VH-CDR3 comprising the sequence set forth in SEQ ID NO:775; a VL-CDR1 comprising the sequence set forth in SEQ ID NO:776; a VL-CDR2 comprising the sequence set forth in SEQ ID NO:777; and a VL-CDR3 comprising the sequence set forth in SEQ ID NO:778.

In another aspect, provided herein is an antibody-drug conjugate comprising: an anti-human Tissue Factor (anti-hTF) antibody, a cytotoxic agent linked to the antibody, and optionally a linker that links the antibody to the cytotoxic agent, wherein the antibody comprises: a VH-CDR1 comprising the sequence set forth in SEQ ID NO:779; a VH-CDR2 comprising the sequence set forth in SEQ ID NO:780; a VH-CDR3 comprising the sequence set forth in SEQ ID NO:781; a VL-CDR1 comprising the sequence set forth in SEQ ID NO:782; a VL-CDR2 comprising the sequence set forth in SEQ ID NO:783; and a VL-CDR3 comprising the sequence set forth in SEQ ID NO:784.

In another aspect, provided herein is an antibody-drug conjugate comprising: an anti-human Tissue Factor (anti-hTF) antibody, a cytotoxic agent linked to the antibody, and optionally a linker that links the antibody to the cytotoxic agent, wherein the antibody comprises: a VH-CDR1 comprising the sequence set forth in SEQ ID NO:785; a VH-CDR2 comprising the sequence set forth in SEQ ID NO:786; a VH-CDR3 comprising the sequence set forth in SEQ ID NO:787; a VL-CDR1 comprising the sequence set forth in SEQ ID NO:788; a VL-CDR2 comprising the sequence set forth in SEQ ID NO:789; and a VL-CDR3 comprising the sequence set forth in SEQ ID NO:790.

In another aspect, provided herein is an antibody-drug conjugate comprising: an anti-human Tissue Factor (anti-hTF) antibody, a cytotoxic agent linked to the antibody, and optionally a linker that links the antibody to the cytotoxic agent, wherein the antibody comprises: a VH-CDR1 comprising the sequence set forth in SEQ ID NO:791; a VH-CDR2 comprising the sequence set forth in SEQ ID NO:792; a VH-CDR3 comprising the sequence set forth in SEQ ID NO:793; a VL-CDR1 comprising the sequence set forth in SEQ ID NO:794; a VL-CDR2 comprising the sequence set forth in SEQ ID NO:795; and a VL-CDR3 comprising the sequence set forth in SEQ ID NO:796.

In another aspect, provided herein is an antibody-drug conjugate comprising: an anti-human Tissue Factor (anti-hTF) antibody, a cytotoxic agent linked to the antibody, and optionally a linker that links the antibody to the cytotoxic agent, wherein the antibody comprises: a VH-CDR1 comprising the sequence set forth in SEQ ID NO:797; a VH-CDR2 comprising the sequence set forth in SEQ ID NO:798; a VH-CDR3 comprising the sequence set forth in SEQ ID NO:799; a VL-CDR1 comprising the sequence set forth in SEQ ID NO:800; a VL-CDR2 comprising the sequence set forth in SEQ ID NO:801; and a VL-CDR3 comprising the sequence set forth in SEQ ID NO:802.

In another aspect, provided herein is an antibody-drug conjugate comprising: an anti-human Tissue Factor (anti-hTF) antibody, a cytotoxic agent linked to the antibody, and optionally a linker that links the antibody to the cytotoxic agent, wherein the antibody comprises: a VH-CDR1 comprising the sequence set forth in SEQ ID NO:803; a VH-CDR2 comprising the sequence set forth in SEQ ID NO:804; a VH-CDR3 comprising the sequence set forth in SEQ ID NO:805; a VL-CDR1 comprising the sequence set forth in SEQ ID NO:806; a VL-CDR2 comprising the sequence set forth in SEQ ID NO:807; and a VL-CDR3 comprising the sequence set forth in SEQ ID NO:808.

In another aspect, provided herein is an antibody-drug conjugate comprising: an anti-human Tissue Factor (anti-hTF) antibody, a cytotoxic agent linked to the antibody, and optionally a linker that links the antibody to the cytotoxic agent, wherein the antibody comprises: a VH-CDR1 comprising the sequence set forth in SEQ ID NO:872; a VH-CDR2 comprising the sequence set forth in SEQ ID NO:873; a VH-CDR3 comprising the sequence set forth in SEQ ID NO:874; a VL-CDR1 comprising the sequence set forth in SEQ ID NO:875; a VL-CDR2 comprising the sequence set forth in SEQ ID NO:876; and a VL-CDR3 comprising the sequence set forth in SEQ ID NO:877.

In another aspect, provided herein is an antibody-drug conjugate comprising: antibody-drug conjugate comprising: an anti-human Tissue Factor (anti-hTF) antibody, a cytotoxic agent linked to the antibody, and optionally a linker that links the antibody to the cytotoxic agent, wherein the antibody comprises: a VH-CDR1 comprising the sequence set forth in SEQ ID NO:878; a VH-CDR2 comprising the sequence set forth in SEQ ID NO:879; a VH-CDR3 comprising the sequence set forth in SEQ ID NO:880; a VL-CDR1 comprising the sequence set forth in SEQ ID NO:881; a VL-CDR2 comprising the sequence set forth in SEQ ID NO:882; and a VL-CDR3 comprising the sequence set forth in SEQ ID NO:883.

In some embodiments, the antibody is human, humanized, or chimeric.

In some embodiments, the antibody binds to human TF with a K_{D} of less than or equal to 50 nM, 10 nM, 5 nM, 1 nM, 0.5 nM or 0.1 nM, as measured by Octet QK384 or Biacore assay.

In some embodiments, the antibody is a monoclonal antibody.

In some embodiments, the antibody is multispecific.

In some embodiments, the antibody is a Fab, Fab′, F(ab′)₂, Fv, scFv, (scFv)₂, single chain antibody molecule, dual variable domain antibody, single variable domain antibody, linear antibody, or V domain antibody.

In some embodiments, the antibody comprises a scaffold, optionally wherein the scaffold is Fc, optionally human Fc. In some embodiments, the antibody comprises a heavy chain constant region of a class selected from IgG, IgA, IgD, IgE, and IgM. In some embodiments, the antibody comprises a heavy chain constant region of the class IgG and a subclass selected from IgG1, IgG2, IgG3, and IgG4. In some embodiments, the antibody comprises a heavy chain constant region of IgG1. In some embodiments, the Fc comprises one or more modifications, wherein the one or more modifications result in increased half-life, increased antibody-dependent cellular cytotoxicity (ADCC), increased antibody-dependent cellular phagocytosis (ADCP), increased complement-dependent cytotoxicity (CDC), or decreased effector function, compared with the Fc without the one or more modifications.

In another aspect, provided herein is an antibody-drug conjugate comprising: an anti-human Tissue Factor (anti-hTF) antibody, a cytotoxic agent linked to the antibody, and optionally a linker that links the antibody to the cytotoxic agent, wherein the antibody competes for binding to human TF with any antibody above.

In another aspect, provided herein is an antibody-drug conjugate comprising: an anti-human Tissue Factor (anti-hTF) antibody, a cytotoxic agent linked to the antibody, and optionally a linker that links the antibody to the cytotoxic agent, wherein the antibody binds the human TF epitope bound by any antibody above.

In some embodiments, the cytotoxic agent comprises an anti-angiogenic agent, a pro-apoptotic agent, an anti-mitotic agent, an anti-kinase agent, an alkylating agent, a hormone, a hormone agonist, a hormone antagonist, a chemokine, a drug, a prodrug, a toxin, an enzyme, an antimetabolite, an antibiotic, an alkaloid, or a radioactive isotope. In some embodiments, the cytotoxic agent comprises at least one of: calicheamycin, camptothecin, carboplatin, irinotecan, SN-38, carboplatin, camptothecan, cyclophosphamide, cytarabine, dacarbazine, docetaxel, dactinomycin, daunorubicin, doxorubicin, doxorubicin, etoposide, idarubicin, topotecan, vinca alkaloid, maytansinoid, maytansinoid analog, pyrrolobenzodiazepine, taxoid, duocarmycin, dolastatin, and auristatin.

In some embodiments, the linker comprises a labile linker, an acid labile linker, a photolabile linker, a charged linker, a disulfide-containing linker, a peptidase-sensitive linker, a β-glucuronide-linker, a dimethyl linker, a thio-ether linker, or a hydrophilic linker. In some embodiments, the linker is a cleavable linker. In some embodiments, the linker is a non-cleavable linker.

In another aspect, provided herein is a pharmaceutical composition comprising any antibody-drug conjugate above and a pharmaceutically acceptable excipient.

In another aspect, provided herein is a method of treating or preventing a disease or condition in a subject in need thereof, comprising administering to the subject an effective amount of any antibody-drug conjugate above or the pharmaceutical composition above.

In some embodiments, the disease or condition is cancer. In some embodiments, the cancer is head and neck cancer. In some embodiments, the cancer is ovarian cancer. In some embodiments, the cancer is gastric cancer. In some embodiments, the cancer is esophageal cancer. In some embodiments, the cancer is cervical cancer. In some embodiments, the cancer is prostate cancer. In some embodiments, the cancer is pancreatic cancer. In some embodiments, the cancer is estrogen receptors negative (ER−), progesterone receptors negative (PR−), and HER2 negative (HER2−) triple negative breast cancer. In some embodiments, the cancer is glioblastoma. In some embodiments, the cancer is lung cancer. In some embodiments, the cancer is bladder cancer. In some embodiments, the cancer is melanoma. In some embodiments, the cancer is kidney cancer.

In some embodiments, the method further comprises administering one or more additional therapeutic agents to the subject. In some embodiments, the additional therapeutic agent is formulated in the same pharmaceutical composition as the antibody-drug conjugate. In some embodiments, the additional therapeutic agent is formulated in a different pharmaceutical composition from the antibody-drug conjugate. In some embodiments, the additional therapeutic agent is administered prior to administering the antibody-drug conjugate. In some embodiments, the additional therapeutic agent is administered after administering the antibody-drug conjugate. In some embodiments, the additional therapeutic agent is administered contemporaneously with the antibody-drug conjugate.

In another aspect, provided herein is a method of detecting TF in a subject having or suspected of having a disease or condition, the method comprising: (a) administering to the subject any antibody-drug conjugate above; and (b) detecting the presence or the level of TF in the subject.

In some embodiments, the disease or condition is cancer. In some embodiments, the cancer is head and neck cancer. In some embodiments, the cancer is ovarian cancer. In some embodiments, the cancer is gastric cancer. In some embodiments, the cancer is esophageal cancer. In some embodiments, the cancer is cervical cancer. In some embodiments, the cancer is prostate cancer. In some embodiments, the cancer is pancreatic cancer. In some embodiments, the cancer is estrogen receptors negative (ER−), progesterone receptors negative (PR−), and HER2 negative (HER2−) triple negative breast cancer. In some embodiments, the cancer is glioblastoma. In some embodiments, the cancer is lung cancer. In some embodiments, the cancer is bladder cancer. In some embodiments, the cancer is melanoma. In some embodiments, the cancer is kidney cancer.

In another aspect, provided herein is a kit comprising any antibody-drug conjugate above or the pharmaceutical composition above and instructions for use.

### BRIEF DESCRIPTION OF THE DRAWINGS

These and other features, aspects, and advantages of the present invention will become better understood with regard to the following description, and accompanying drawings, where:

FIGS. 1A and 1B show binding of anti-TF antibodies to human TF-positive cells. FIG. 1A shows the median fluorescence intensity (MFI) of antibody bound to HCT-116 cells plotted against concentrations of antibodies from groups 1, 25, and 29 and the reportable cell EC₅₀. FIG. 1B shows the median fluorescence intensity of antibody bound to HCT-116 cells plotted against concentrations of antibodies from groups 39, 43, and 54 and the reportable cell EC₅₀. The isotype control in FIG. 1B applies to both FIGS. 1A and 1B.

FIGS. 2A and 2B show binding of anti-TF antibodies to mouse TF-positive cells. FIG. 2A shows the median fluorescence intensity (MFI) of antibody bound to CHO cells recombinantly expressing mouse TF (CHO-mTF) plotted against concentrations of antibodies from groups 1, 25, and 29 and the reportable cell EC₅₀. FIG. 2B shows the median fluorescence intensity of antibody bound to CHO-mTF cells plotted against concentrations of antibodies from groups 39, 43, and 54 and the reportable cell EC₅₀. The isotype control in FIG. 2B applies to both FIGS. 2A and 2B.

FIGS. 3A and 3B show thrombin generation in the presence of anti-TF antibody. FIG. 3A shows Peak IIa/Thrombin generation (% Peak IIa) as measured by the Thrombin Generation Assay (TGA) without antibody incubation prior to addition of calcium and thrombin substrate in the presence of titrations of anti-TF antibodies from groups 1, 25, 29, 39, 43, and 54. FIG. 3B shows Peak IIa/Thrombin generation (% Peak IIa) as measured by the Thrombin Generation Assay (TGA) with a 10-min antibody incubation prior to addition of calcium and thrombin substrate in the presence of titrations of anti-TF antibodies from groups 1, 25, 29, 39, 43, and 54.

FIGS. 4A and 4B show FXa conversion in the presence of anti-TF antibody. FIG. 4A shows the percentage of FXa conversion (% FXa) by TF:FVIIa in MDA-MB-231 cells in the presence of titrations of anti-TF antibodies from groups 1, 25, and 29. FIG. 4B shows the percentage of FXa conversion (% FXa) by TF:FVIIa in MDA-MB-231 cells in the presence of titrations of anti-TF antibodies from groups 39, 43, and 54. % FXa conversion at a reported concentration is calculated relative to an antibody-free FXa conversion reaction. The isotype control in FIG. 4B applies to both FIGS. 4A and 4B.

FIGS. 5A and 5B show FVIIa binding in the presence of anti-TF antibody. FIG. 5A shows the percentage of FVIIa binding (% FVIIa) in TF-positive MDA-MB-231 cells in the presence of titrations of anti-TF antibodies from groups 1, 25, and 29. FIG. 5B shows the percentage of FVIIa binding (% FVIIa) in MDA-MB-231 cells in the presence of titrations of anti-TF antibodies from groups 39, 43, and 54. % FVIIa binding at a reported concentration is calculated relative to antibody-free FVIIa binding. The isotype control in FIG. 5B applies to both FIGS. 5A and 5B.

FIGS. 6A, 6B, 6C, and 6D show FVIIa-dependent TF signaling in the presence of anti-TF antibody. FIG. 6A shows the concentration of IL8 (IL8 conc) in MDA-MB-231 cells in the presence of titrations of anti-TF antibodies from groups 1, 25, and 29. FIG. 6B shows the concentration of IL8 (IL8 conc) in MDA-MB-231 cells in the presence of titrations of anti-TF antibodies from groups 39, 43, and 54. The control in FIG. 6B applies to both FIGS. 6A and 6B. FIG. 6C shows the concentration of GM-CSF (GM-CSF conc) in MDA-MB-231 cells in the presence of titrations of anti-TF antibodies from groups 1, 25, and 29. FIG. 6D shows the concentration of GM-CSF (GM-CSF conc) in MDA-MB-231 cells in the presence of titrations of anti-TF antibodies from groups 39, 43, and 54. The control in FIG. 6D applies to both FIGS. 6C and 6D.

FIGS. 7A and 7B show internalization of anti-TF antibody by TF-positive cells. FIG. 7A shows the cell viability of TF-positive A431 cell cultures after the addition of an anti-TF antibody from groups 1, 25, and 29 and a secondary antibody against the human Fc conjugated to mono-methyl auristatin F (MMAF). FIG. 7B shows the cell viability of TF-positive A431 cell cultures after the addition of an anti-TF antibody from groups 39, 43, and 54 and a secondary antibody against the human Fc conjugated to mono-methyl auristatin F (MMAF). The isotype control in FIG. 7B applies to both FIGS. 7A and 7B.

FIGS. 8A and 8B show thrombin generation in the presence of anti-TF antibody. FIG. 8A shows Peak IIa/Thrombin generation (% Peak IIa) as measured by the Thrombin Generation Assay (TGA) without antibody incubation prior to addition of calcium and thrombin substrate in the presence of titrations of anti-TF antibodies from groups 25, 39, 43, and anti-TF M1593. FIG. 8B shows Peak IIa/Thrombin generation (% Peak IIa) as measured by the Thrombin Generation Assay (TGA) with a 10-min antibody incubation prior to addition of calcium and thrombin substrate in the presence of titrations of anti-TF antibodies from groups 25, 39, 43, and anti-TF M1593.

FIGS. 9A and 9B show anti-TF ADC-induced cell death in TF-positive cells. FIG. 9A shows cell viability of TF-positive A431 cells after a 3-day incubation with titrations of anti-TF antibodies conjugated to MC-vc-PAB-MMAE (DAR of 3-4). FIG. 9B shows cell viability of TF-positive HPAF-II cells after a 4-day incubation with titrations of anti-TF antibodies conjugated to MC-vc-PAB-MMAE (DAR of 3-4).

FIGS. 10A and 10B show the effect of anti-TF ADCs on tumor size in xenograft models. FIG. 10A shows the efficacy of anti-TF ADCs in the A431 xenograft model. FIG. 10B shows the efficacy of anti-TF ADCs in the HPAF-II xenograft model.

The arrows indicate treatments with ADC or vehicle (PBS) dosed at 5 mg/kg once per week for 3 weeks.

FIG. 11 shows the effect of anti-TF ADCs on tumor size in a head and neck cancer patient-derived xenograft model. The arrows indicate treatments with anti-TF ADC or IgG1 control ADC dosed at 5 mg/kg once per week for 2 weeks.

FIGS. 12A and 12B show binding of anti-TF antibodies to human TF-positive cancer cells. FIG. 12A shows the median fluorescence intensity (MFI) of antibody bound to A431 cells plotted against concentrations of antibodies from groups 1, 25, 29, 39, 43, and 54. Reportable cell EC₅₀'s and their 95% confidence intervals are listed. FIG. 12B shows the median fluorescence intensity of antibody bound to MDA-MB-231 cells plotted against concentrations of antibodies from groups 25, 29, 39, and 43. Reportable cell EC₅₀'s and their 95% confidence intervals are listed.

FIGS. 13A, 13B and 13C show thrombin generation in the presence of anti-TF antibody. FIG. 13A shows the thrombin generation curves in the absence or presence of 100 nM anti-TF antibodies from groups 1, 25, and 29 and previously generated anti-TF antibodies TF-011, 5G9, and 10H10 (samples on plate 1 of Table 44). FIG. 13B shows the thrombin generation curves in the absence or presence of 100 nM anti-TF antibodies from groups 39, 43, and 54 (samples on plate 2 of Table 44). FIG. 13C shows the peak thrombin concentration in the absence or presence of titrations of anti-TF antibodies. The mean of a triplicate data set is shown. The standard deviation of the mean is listed in Table 44.

FIGS. 14A and 14B show TF:FVIIa-dependent FXa Conversion and FVII binding in the presence of anti-TF antibodies TF-011, 5G9, and 10H10. FIG. 14A shows TF:FVIIa-dependent conversion of FX into FXa on the cell surface of MDA-MB-231 cells in the absence or presence of titrations of anti-TF antibodies TF-011, 5G9 and 10H10. FIG. 14B shows FVII binding in the absence or presence of titrations of anti-TF antibodies TF-011, 5G9 and 10H10 after pre-incubation of MDA-MB-231 cells with the anti-TF antibodies. For antibodies that exhibited no less than 25% competition with FVII, the IC₅₀ is reported.

FIGS. 15A and 15B show percent binding (% Binding) of A488-conjugated 25A3 anti-TF antibody to MDA-MB-231 cells after pre-incubation of the cells with titrations of unlabeled competitor antibodies. FIG. 15A shows percent binding of 25A3 after pre-incubation with unlabeled competitor antibodies from groups 1, 25, 29, 39, 43, and 54. FIG. 15B shows percent binding of 25A3 after pre-incubation with unlabeled competitor antibodies TF-011, 5G9, and 10H10. The IC₅₀ value of antibodies that compete with 25A3 is listed.

FIGS. 16A and 16B show percent binding (% Binding) of A488-conjugated 43D7 anti-TF antibody to MDA-MB-231 cells after pre-incubation of the cells with titrations of unlabeled competitor antibodies. FIG. 16A shows percent binding of 43D7 after pre-incubation with unlabeled competitor antibodies from groups 1, 25, 29, 39, 43, and 54. FIG. 16B shows percent binding of 43D7 after pre-incubation with unlabeled competitor antibodies TF-011, 5G9, and 10H10. The IC₅₀ value of antibodies that compete with 43D7 is listed.

FIGS. 17A and 17B show percent binding (% Binding) of A488-conjugated 39A anti-TF antibody to MDA-MB-231 cells after pre-incubation of the cells with titrations of unlabeled competitor antibodies. FIG. 17A shows percent binding of 39A after pre-incubation with unlabeled competitor antibodies from groups 1, 25, 29, 39, 43, and 54. FIG. 17B shows percent binding of 39A after pre-incubation with unlabeled competitor antibodies TF-011, 5G9, and 10H10. The IC₅₀ value of antibodies that compete with 39A is listed.

FIGS. 18A, 18B, and 18C show the internalization of anti-TF antibodies as measured by cell viability assay and internalization assay. FIG. 18A shows cell viability of TF-positive A431 cell cultures three days after titrations of anti-TF antibodies. FIG. 18B shows cell viability of TF-positive A431 cell cultures three days after titrations of anti-TF antibodies complexed with a Fab fragment against the human Fc conjugated to mono-methyl auristatin F (Fab:MMAF). The IC₅₀ of the anti-TF antibody Fab:MMAF complexes is listed. FIG. 18C shows internalization of anti-TF antibodies conjugated to A488. Percent internalization of A488-conjugated anti-TF antibodies at 4 h is listed.

FIGS. 19A, 19B, and 19C show the binding of anti-TF antibodies and ADCs to human TF-positive HCT-116 cells. FIG. 19A shows the binding of anti-TF antibodies HCT-116 cells. FIG. 19B shows the binding of anti-TF ADCs to HCT-116 cells. FIG. 19C lists reportable cell EC₅₀'s and their 95% confidence intervals.

FIGS. 20A, 20B, and 20C show cell viability of A431 cells after titrations of anti-TF ADCs. FIG. 20A shows the cell viability after titrations of anti-TF ADCs with a continuous 72 h incubation. FIG. 20B shows the cell viability after titrations of anti-TF ADCs with a 4 h incubation followed by removal of excess ADC and culture for another 68 h. FIG. 20C lists the reportable IC₅₀ values of ADCs.

FIGS. 21A, 21B, and 21C show the effect of FVIIa on the in vitro efficacy of anti-TF ADCs. FIG. 21A shows the cell viability after titrations of anti-TF ADCs with a 4 h incubation followed by removal of excess ADC and culture for another 68 h in the absence of FVIIa. FIG. 21B shows the cell viability after titrations of anti-TF ADCs with a 4 h incubation followed by removal of excess ADC and culture for another 68 h in the presence of FVIIa. FIG. 21C lists the reportable IC₅₀ values.

FIGS. 22A, 22B, 22C, 22D, and 22E show cell viability of additional cancer cell lines after titrations of anti-TF ADCs. FIG. 22A shows the TF copy number in various cell lines with the anti-TF antibody 5G9. The standard error of the mean and the number of measurements (n) are also presented. FIG. 22B shows the cell viability of HCT-116 cells after 72 h culture in the absence or presence of titrations of anti-TF MMAE ADCs. FIG. 22C shows the cell viability of CHO cells after 72 h culture in the absence or presence of titrations of anti-TF MMAE ADCs. FIG. 22D shows the cell viability of MDA-MB-231 cells after 5-day culture in the absence or presence of titrations of anti-TF MMAE ADCs. FIG. 22E shows the cell viability of HPAF-II cells after 5-day culture in the absence or presence of titrations of anti-TF MMAE ADCs.

FIGS. 23A and 23B show staining of the microtubule network after treatment with anti-TF 25A3 MMAE ADC (25A3-vc-MMAE) or isotype control MMAE ADC (isotype ctrl-vc-MMAE). FIG. 23A shows staining of the microtubule network of A431 cells after treatment. FIG. 23B shows staining of the microtubule network of HPAF-II cells after treatment. Scale bar, 10 microns.

FIGS. 24A and 24B show the TF expression after cytokine treatment and the effect of anti-TF ADCs on the viability of cytokine-treated human umbilical vein endothelial cells (HUVECs). FIG. 24A shows copy numbers of surface TF on HUVECs treated with or without an inflammatory cytokine cocktail for 3, 6, or 20 h prior to analysis. FIG. 24B shows cell viability of inflammatory cytokine-treated HUVEC cultures after 4 days of culture in the presence of titrations of anti-TF or isotype-control MMAE ADCs.

FIGS. 25A, 25B, and 25C show the quantitation of the G2/M arrest in HUVECs or HCT-116 cells treated for 24 h with titrations of anti-TF ADCs. FIG. 25A shows the percentage of pH3-positive cells (% pH3) with titrations of anti-TF ADCs of HUVECs in the absence of inflammatory cytokines. FIG. 25B shows the percentage of pH3-positive cells (% pH3) with titrations of anti-TF ADCs of HUVECs in the presence of inflammatory cytokines. FIG. 25C shows the percentage of pH3-positive cells (% pH3) with titrations of anti-TF ADCs of HCT-116 cells.

FIGS. 26A and 26B show the percentage of pH3-positive HCT-116 cells analyzed by flow cytometry with or without anti-TF ADC treatment. FIG. 26A shows the pH3 versus DNA content dot plot after treatment of 10 nM Isotype-vc-MMAE. FIG. 26B shows the pH3 versus DNA content dot plot after treatment of 10 nM 25A-vc-MMAE.

FIGS. 27A and 27B show the sensitivity of HUVECs and HCT-116 cells to MMAE. FIG. 27A shows the percentage of pH3-positive HUVECs (% pH3) in the absence or presence of 24 h of MMAE treatment. FIG. 27B shows the percentage of pH3-positive HCT-116 cells (% pH3) in the absence or presence of 24 h of MMAE treatment.

FIG. 28 shows the analysis of Erk phosphorylation by Western blotting with an anti-phospho-Erk1/2 antibody and an anti-Erk1/2 antibody. The values of pErk induction are listed.

FIGS. 29A, 29B, and 29C show antibody-dependent cellular cytotoxicity (ADCC) reporter luminescence after a 6 h incubation of the reporter Jurkat cell line with TF-positive A431 cells. FIG. 29A shows the ADCC reporter luminescence in the absence or presence of titrations anti-TF antibodies. FIG. 29B shows the ADCC reporter luminescence in the absence or presence of titrations anti-TF ADCs. FIG. 29C lists the ADCC reporter luminescence EC₅₀ values for each anti-TF antibody or ADC.

FIGS. 30A and 30B show in vivo efficacy of anti-TF ADCs in HPAF-II xenograft model. FIG. 30A shows the mean tumor volume after weekly treatment of an anti-TF ADC at 5 mg/kg for 3 weeks. FIG. 30B shows the mean tumor volume after weekly treatment of an anti-TF ADC at 2 mg/kg for 2 weeks. The mean tumor volumes (Mean) and tumor growth inhibition (TGI) percentages on day 21 are listed. The P-values for the mean tumor volume comparison between each ADC and the vehicle control are also listed. In addition, the number of partial regression (PR), complete regression (CR), and tumor-free survivor (TFS) animals at the end of the study (day 59 for FIG. 30A and day 39 for FIG. 30B) are also listed.

FIGS. 31A and 31B show in vivo efficacy of anti-TF ADCs in MDA-MB-231 xenograft model. FIG. 31A shows the mean tumor volume after weekly treatment of an anti-TF ADC at 4 mg/kg for 2 weeks. FIG. 31B shows the mean tumor volume after weekly treatment of an anti-TF ADC at 2 mg/kg for 2 weeks. The mean tumor volume and tumor growth inhibition on day 25 (FIG. 31A) and day 27 (FIG. 31B) are listed. The P-values for the mean tumor volume comparison between each ADC and the vehicle control are also listed. In addition, the number of partial regression (PR), complete regression (CR), and tumor-free survivor (TFS) animals at the end of the study (day 49 for FIG. 31A and day 41 for FIG. 31B) are also listed.

FIG. 32 shows the mean tumor volume after weekly treatment of unconjugated anti-TF antibodies at 10 mg/kg for 2 weeks in the HPAF-II xenograft model. The mean tumor volume on day 15 is listed.

FIGS. 33A, 33B, and 33C show in vivo efficacy of anti-TF ADCs in patient-derived xenograft (PDX) models. FIG. 33A shows the mean tumor volume in the PDX model of a head and neck carcinoma after treatment of an anti-TF ADC. FIG. 33B shows the mean tumor volume in the PDX model of an ovarian carcinoma after treatment of an anti-TF ADC. FIG. 33C shows the mean tumor volume in the PDX model of a gastric adenocarcinoma after treatment of an anti-TF ADC. The mean tumor volume and tumor growth inhibition on day 44 (FIG. 33A), day 15 (FIG. 33B), and day 25 (FIG. 33C) are listed. The P-values for the mean tumor volume comparison between each ADC and the isotype control are also listed. In addition, the number of partial responder (PR), complete responder (CR), and tumor free survivor (TFS) animals at the end of the study (day 60 for FIG. 33A and day 46 for FIGS. 33B-C) are also listed.

FIGS. 34A and 34B show the change in lesion size after administration of anti-TF antibody in a swine choroidal neovascularization (CNV) model. FIG. 34A shows the percentage change in lesion size from day 7 (baseline) to day 14 as measured by Fluorescein Angiography (FA) after intravitreal administration of anti-TF antibodies 25G9, 43D8, 1G, and 29D respectively. FIG. 34B shows the percentage change in lesion size from day 7 (baseline) to day 28 as measured by Fluorescein Angiography (FA) after intravitreal administration of anti-TF antibodies 25G9, 43D8, 1G, and 29D respectively.

FIG. 35 shows the change in lesion size in a swine choroidal neovascularization (CNV) model from day 7 (baseline) to day 28 as measured by Fluorescein Angiography (FA) after intravitreal administration of anti-TF antibodies 25G9 at 600 ug, 2 mg and 4 mg respectively.

FIG. 36 shows Clustal Omega alignment of chimeric TF constructs. Rat sequences are highlighted in bold. An “* (asterisk)” indicates positions which have a single, fully conserved residue. A “: (colon)” indicates conservation between groups of strongly similar properties—roughly equivalent to scoring>0.5 in the Gonnet Percent Accepted Mutation 250 matrix. A “. (period)” indicates conservation between groups of weakly similar properties—roughly equivalent to scoring=<0.5 and >0 in the Gonnet Percent Accepted Mutation 250 matrix.

FIG. 37 shows Clustal Omega alignment of chimeric TF constructs. Human sequences are highlighted in bold. An “* (asterisk)” indicates positions which have a single, fully conserved residue. A “: (colon)” indicates conservation between groups of strongly similar properties—roughly equivalent to scoring>0.5 in the Gonnet Percent Accepted Mutation 250 matrix. A “. (period)” indicates conservation between groups of weakly similar properties—roughly equivalent to scoring=<0.5 and >0 in the Gonnet Percent Accepted Mutation 250 matrix.

FIG. 38 shows Clustal Omega alignment of chimeric TF constructs. Rat sequences are highlighted in bold. An “* (asterisk)” indicates positions which have a single, fully conserved residue. A “: (colon)” indicates conservation between groups of strongly similar properties—roughly equivalent to scoring>0.5 in the Gonnet Percent Accepted Mutation 250 matrix. A “. (period)” indicates conservation between groups of weakly similar properties—roughly equivalent to scoring=<0.5 and >0 in the Gonnet Percent Accepted Mutation 250 matrix.

FIGS. 39A-F show the titration curves of anti-TF antibodies from lineages 25 and 43, h5G9, and 10H10 on select TF constructs. FIG. 39A shows the titration curves of anti-TF antibodies on human TF construct. FIG. 39B shows the titration curves of anti-TF antibodies on rat TF construct. FIG. 39C shows the titration curves of anti-TF antibodies on chimeric human-rat TF construct hTF_K68N. FIG. 39D shows the titration curves of anti-TF antibodies on chimeric human-rat TF construct hTF_K149N. FIG. 39E shows the titration curves of anti-TF antibodies on chimeric human-rat TF construct hTF_N171H_T197K. FIG. 39F shows the titration curves of anti-TF antibodies on chimeric rat-human TF construct r141-194_h.

### DETAILED DESCRIPTION

### 1. Definitions

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise.

The term “about” indicates and encompasses an indicated value and a range above and below that value. In certain embodiments, the term “about” indicates the designated value±10%, ±5%, or ±1%. In certain embodiments, where applicable, the term “about” indicates the designated value(s)±one standard deviation of that value(s).

The terms “Tissue Factor,” “TF,” “platelet tissue factor,” “factor III,” “thromboplastin,” and “CD142” are used interchangeably herein to refer to TF, or any variants (e.g., splice variants and allelic variants), isoforms, and species homologs of TF that are naturally expressed by cells, or that are expressed by cells transfected with a TF gene. In some aspects, the TF protein is a TF protein naturally expressed by a primate (e.g., a monkey or a human), a rodent (e.g., a mouse or a rat), a dog, a camel, a cat, a cow, a goat, a horse, a pig or a sheep. In some aspects, the TF protein is human TF (hTF; SEQ ID NO:809). In some aspects, the TF protein is cynomolgus TF (cTF; SEQ ID NO:813). In some aspects, the TF protein is mouse TF (mTF; SEQ ID NO:817). In some aspects, the TF protein is pig TF (pTF; SEQ ID NO:824). TF is a cell surface receptor for the serine protease factor VIIa. It is often times constitutively expressed by certain cells surrounding blood vessels and in some disease settings.

The term “antibody-drug conjugate” or “ADC” refers to a conjugate comprising an antibody conjugated to one or more cytotoxic agents, optionally through one or more linkers. The term “anti-TF antibody-drug conjugate” or “anti-TF ADC” refers to a conjugate comprising an anti-TF antibody conjugated to one or more cytotoxic agents, optionally through one or more linkers.

The term “cytotoxic agent,” as used herein, refers to a substance that inhibits or prevents a cellular function and/or causes cell death or destruction. The cytotoxic agent can be an anti-angiogenic agent, a pro-apoptotic agent, an anti-mitotic agent, an anti-kinase agent, an alkylating agent, a hormone, a hormone agonist, a hormone antagonist, a chemokine, a drug, a prodrug, a toxin, an enzyme, an antimetabolite, an antibiotic, an alkaloid, or a radioactive isotope. Exemplary cytotoxic agents include calicheamycin, camptothecin, carboplatin, irinotecan, SN-38, carboplatin, camptothecan, cyclophosphamide, cytarabine, dacarbazine, docetaxel, dactinomycin, daunorubicin, doxorubicin, doxorubicin, etoposide, idarubicin, topotecan, vinca alkaloid, maytansinoid, maytansinoid analog, pyrrolobenzodiazepine, taxoid, duocarmycin, dolastatin, auristatin, and derivatives thereof.

A “linker” refers to a molecule that connects one composition to another, e.g., an antibody to an agent. Linkers described herein can conjugate an antibody to a cytotoxic agent. Exemplary linkers include a labile linker, an acid labile linker, a photolabile linker, a charged linker, a disulfide-containing linker, a peptidase-sensitive linker, a -glucuronide-linker, a dimethyl linker, a thio-ether linker, and a hydrophilic linker. A linker can be cleavable or non-cleavable.

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (V_{H}) and a heavy chain constant region (CH). The heavy chain constant region typically comprises three domains, abbreviated CH1, CH2, and CH3. Each light chain typically comprises a light chain variable region (V_{L}) and a light chain constant region. The light chain constant region typically comprises one domain, abbreviated CL.

The term “antibody” is used herein in its broadest sense and includes certain types of immunoglobulin molecules comprising one or more antigen-binding domains that specifically bind to an antigen or epitope. An antibody specifically includes intact antibodies (e.g., intact immunoglobulins), antibody fragments, and multi-specific antibodies.

The term “alternative scaffold” refers to a molecule in which one or more regions may be diversified to produce one or more antigen-binding domains that specifically bind to an antigen or epitope. In some embodiments, the antigen-binding domain binds the antigen or epitope with specificity and affinity similar to that of an antibody. Exemplary alternative scaffolds include those derived from fibronectin (e.g., Adnectins™), the β-sandwich (e.g., iMab), lipocalin (e.g., Anticalins®), EETI-II/AGRP, BPTI/LACI-D1/ITI-D2 (e.g., Kunitz domains), thioredoxin peptide aptamers, protein A (e.g., Affibody®), ankyrin repeats (e.g., DARPins), gamma-B-crystallin/ubiquitin (e.g., Affilins), CTLD3 (e.g., Tetranectins), Fynomers, and (LDLR-A module) (e.g., Avimers). Additional information on alternative scaffolds is provided in Binz et al., Nat. Biotechnol., 2005 23:1257-1268; Skerra, Current Opin. in Biotech., 2007 18:295-304; and Silacci et al., J. Biol. Chem., 2014, 289:14392-14398; each of which is incorporated by reference in its entirety.

The term “antigen-binding domain” means the portion of an antibody that is capable of specifically binding to an antigen or epitope. One example of an antigen-binding domain is an antigen-binding domain formed by a V_{H}-V_{L} dimer of an antibody. Another example of an antigen-binding domain is an antigen-binding domain formed by diversification of certain loops from the tenth fibronectin type III domain of an Adnectin. Antigen-binding domains can be found in various contexts including antibodies and chimeric antigen receptors (CARs), for example CARs derived from antibodies or antibody fragments such as scFvs.

The terms “full length antibody,” “intact antibody,” and “whole antibody” are used herein interchangeably to refer to an antibody having a structure substantially similar to a naturally occurring antibody structure and having heavy chains that comprise an Fc region. For example, when used to refer to an IgG molecule, a “full length antibody” is an antibody that comprises two heavy chains and two light chains.

The term “Fc region” means the C-terminal region of an immunoglobulin heavy chain that, in naturally occurring antibodies, interacts with Fc receptors and certain proteins of the complement system. The structures of the Fc regions of various immunoglobulins, and the glycosylation sites contained therein, are known in the art. See Schroeder and Cavacini, J. Allergy Clin. Immunol., 2010, 125:541-52, incorporated by reference in its entirety. The Fc region may be a naturally occurring Fc region, or an Fc region modified as described in the art or elsewhere in this disclosure.

The V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and influence antigen specificity and binding affinity of the antibody. See Kabat et al., Sequences of Proteins of Immunological Interest 5th ed. (1991) Public Health Service, National Institutes of Health, Bethesda, MD, incorporated by reference in its entirety.

A “Complementary Determining Region (CDR)” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. CDRs are variable region sequences interspersed within the framework region sequences. CDRs are well recognized in the art and have been defined by, for example, Kabat as the regions of most hypervariability within the antibody variable (V) domains. See Kabat et al., J Biol Chem, 1977, 252:6609-6616 and Kabat, Adv Protein Chem, 1978, 32:1-75, each of which is incorporated by reference in its entirety. CDRs have also been defined structurally by Chothia as those residues that are not part of the conserved β-sheet framework, and thus are able to adapt different conformations. See Chothia and Lesk, J Mol Biol, 1987, 196:901-917, incorporated by reference in its entirety. Both the Kabat and Chothia nomenclatures are well known in the art. AbM, Contact and IMGT also defined CDRs. CDR positions within a canonical antibody variable domain have been determined by comparison of numerous structures. See Morea et al., Methods, 2000, 20:267-279 and Al-Lazikani et al., J Mol Biol, 1997, 273:927-48, each of which is incorporated by reference in its entirety. Because the number of residues within a hypervariable region varies in different antibodies, additional residues relative to the canonical positions are conventionally numbered with a, b, c and so forth next to the residue number in the canonical variable domain numbering scheme (Al-Lazikani et al., supra). Such terminology is well known to those skilled in the art.

A number of hypervariable region delineations are in use and are included herein. The Kabat CDRs are based on sequence variability and are the most commonly used. See Kabat et al. (1992) Sequences of Proteins of Immunological Interest, DIANE Publishing: 2719, incorporated by reference in its entirety. Chothia refers instead to the location of the structural loops (Chothia and Lesk, supra). The AbM hypervariable regions represent a compromise between the Kabat CDRs and Chothia structural loops, and are used by Oxford Molecular's AbM antibody modeling software. The Contact hypervariable regions are based on an analysis of the available complex crystal structures. The residues from each of these hypervariable regions are noted in Table 1.

More recently, a universal numbering system ImMunoGeneTics (IMGT) Information System™ has been developed and widely adopted. See Lefranc et al., Dev Comp Immunol, 2003, 27:55-77, incorporated by reference in its entirety. IMGT is an integrated information system specializing in immunoglobulins (IG), T cell receptors (TR) and major histocompatibility complex (MHIC) of human and other vertebrates. The IMGT CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. Correspondence between the Kabat, Chothia and IMGT numbering is also well known in the art (Lefranc et al., supra). An Exemplary system, shown herein, combines Kabat and Chothia CDR definitions.

TABLE 1

| | Exemplary | | | | | |
|---|---|---|---|---|---|---|
| | (Kabat + | | | | | |
| | Chothia) | Kabat | Chothia | AbM | Contact | IMGT |
| VH CDR1 | 26-35 | 31-35 | 26-32 | 26-35 | 30-35 | 27-38 |
| VH CDR2 | 50-65 | 50-65 | 52a-55 | 50-58 | 47-58 | 56-65 |
| VH CDR3 | 95-102 | 95-102 | 96-101 | 95-102 | 93-101 | 105-117 |
| VL CDR1 | 24-34 | 24-34 | 26-32 | 24-34 | 30-36 | 27-38 |
| VL CDR2 | 50-56 | 50-56 | 50-52 | 50-56 | 46-55 | 56-65 |
| VL CDR3 | 89-97 | 89-97 | 91-96 | 89-97 | 89-96 | 105-117 |

The light chain from any vertebrate species can be assigned to one of two types, called kappa (κ) and lambda (λ), based on the sequence of its constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The term “constant region” or “constant domain” refers to a carboxy terminal portion of the light and heavy chain which is not directly involved in binding of the antibody to antigen but exhibits various effector function, such as interaction with the Fc receptor. The terms refer to the portion of an immunoglobulin molecule having a more conserved amino acid sequence relative to the other portion of the immunoglobulin, the variable domain, which contains the antigen-binding site. The constant domain contains the CH1, CH2 and CH3 domains of the heavy chain and the CL domain of the light chain.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

An “antibody fragment” comprises a portion of an intact antibody, such as the antigen-binding or variable region of an intact antibody. Antibody fragments include, for example, Fv fragments, Fab fragments, F(ab′)₂ fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H1}) of the heavy chain. Fab fragments may be generated, for example, by recombinant methods or by papain digestion of a full-length antibody.

“F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with β-mercaptoethanol.

“Single-chain Fv” or “sFv” or “scFv” antibody fragments comprise a V_{H} domain and a V_{L} domain in a single polypeptide chain. The V_{H} and V_{L} are generally linked by a peptide linker. See Plückthun A. (1994). Any suitable linker may be used. In some embodiments, the linker is a (GGGGS)ₙ (SEQ ID NO:823). In some embodiments, n=1, 2, 3, 4, 5, or 6. See Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety.

“scFv-Fc” fragments comprise an scFv attached to an Fc domain. For example, an Fc domain may be attached to the C-terminal of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used.

The term “single domain antibody” refers to a molecule in which one variable domain of an antibody specifically binds to an antigen without the presence of the other variable domain. Single domain antibodies, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety. Single domain antibodies are also known as sdAbs or nanobodies.

A “multispecific antibody” is an antibody that comprises two or more different antigen-binding domains that collectively specifically bind two or more different epitopes. The two or more different epitopes may be epitopes on the same antigen (e.g., a single TF molecule expressed by a cell) or on different antigens (e.g., a TF molecule and a non-TF molecule). In some aspects, a multi-specific antibody binds two different epitopes (i.e., a “bispecific antibody”). In some aspects, a multi-specific antibody binds three different epitopes (i.e., a “trispecific antibody”). In some aspects, a multi-specific antibody binds four different epitopes (i.e., a “quadspecific antibody”). In some aspects, a multi-specific antibody binds five different epitopes (i.e., a “quintspecific antibody”). In some aspects, a multi-specific antibody binds 6, 7, 8, or more different epitopes. Each binding specificity may be present in any suitable valency. Examples of multispecific antibodies are provided elsewhere in this disclosure.

A “monospecific antibody” is an antibody that comprises one or more binding sites that specifically bind to a single epitope. An example of a monospecific antibody is a naturally occurring IgG molecule which, while divalent (i.e., having two antigen-binding domains), recognizes the same epitope at each of the two antigen-binding domains. The binding specificity may be present in any suitable valency.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human antibody (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

An “isolated antibody” or “isolated nucleic acid” is an antibody or nucleic acid that has been separated and/or recovered from a component of its natural environment. Components of the natural environment may include enzymes, hormones, and other proteinaceous or nonproteinaceous materials. In some embodiments, an isolated antibody is purified to a degree sufficient to obtain at least 15 residues of N-terminal or internal amino acid sequence, for example by use of a spinning cup sequenator. In some embodiments, an isolated antibody is purified to homogeneity by gel electrophoresis (e.g., SDS-PAGE) under reducing or nonreducing conditions, with detection by Coomassie blue or silver stain. In some embodiments, an isolated antibody may include an antibody in situ within recombinant cells, since at least one component of the antibody's natural environment is not present. In some aspects, an isolated antibody or isolated nucleic acid is prepared by at least one purification step. In some embodiments, an isolated antibody or isolated nucleic acid is purified to at least 80%, 85%, 90%, 95%, or 99% by weight. In some embodiments, an isolated antibody or isolated nucleic acid is purified to at least 80%, 85%, 90%, 95%, or 99% by volume. In some embodiments, an isolated antibody or isolated nucleic acid is provided as a solution comprising at least 85%, 90%, 95%, 98%, 99% to 100% antibody or nucleic acid by weight. In some embodiments, an isolated antibody or isolated nucleic acid is provided as a solution comprising at least 85%, 90%, 95%, 98%, 99% to 100% antibody or nucleic acid by volume.

“Affinity” refers to the strength of the sum total of non-covalent interactions between a single binding site of a molecule (e.g., an antibody) and its binding partner (e.g., an antigen or epitope). Unless indicated otherwise, as used herein, “affinity” refers to intrinsic binding affinity, which reflects a 1:1 interaction between members of a binding pair (e.g., antibody and antigen or epitope). The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein, such as surface plasmon resonance (SPR) technology (e.g., BIACORE®) or biolayer interferometry (e.g., FORTEBIO®).

With regard to the binding of an antibody to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the antibody to the target molecule is competitively inhibited by the control molecule. In some aspects, the affinity of a TF antibody for a non-target molecule is less than about 50% of the affinity for TF. In some aspects, the affinity of a TF antibody for a non-target molecule is less than about 40% of the affinity for TF. In some aspects, the affinity of a TF antibody for a non-target molecule is less than about 30% of the affinity for TF. In some aspects, the affinity of a TF antibody for a non-target molecule is less than about 20% of the affinity for TF. In some aspects, the affinity of a TF antibody for a non-target molecule is less than about 10% of the affinity for TF. In some aspects, the affinity of a TF antibody for a non-target molecule is less than about 1% of the affinity for TF. In some aspects, the affinity of a TF antibody for a non-target molecule is less than about 0.1% of the affinity for TF.

The term “k_{d}” (sec⁻¹), as used herein, refers to the dissociation rate constant of a particular antibody-antigen interaction. This value is also referred to as the k_{off} value.

The term “kₐ” (M⁻¹× sec⁻¹), as used herein, refers to the association rate constant of a particular antibody-antigen interaction. This value is also referred to as the kₒₙ value.

The term “K_{D}” (M), as used herein, refers to the dissociation equilibrium constant of a particular antibody-antigen interaction. K_{D}=k_{d}/kₐ. In some embodiments, the affinity of an antibody is described in terms of the K_{D} for an interaction between such antibody and its antigen. For clarity, as known in the art, a smaller K_{D} value indicates a higher affinity interaction, while a larger K_{D} value indicates a lower affinity interaction.

The term “K_{A}” (M⁻¹), as used herein, refers to the association equilibrium constant of a particular antibody-antigen interaction. K_{A}=kₐ/k_{d}.

An “affinity matured” antibody is an antibody with one or more alterations (e.g., in one or more CDRs or FRs) relative to a parent antibody (i.e., an antibody from which the altered antibody is derived or designed) that result in an improvement in the affinity of the antibody for its antigen, compared to the parent antibody which does not possess the alteration(s). In some embodiments, an affinity matured antibody has nanomolar or picomolar affinity for the target antigen. Affinity matured antibodies may be produced using a variety of methods known in the art. For example, Marks et al. (Bio/Technology, 1992, 10:779-783, incorporated by reference in its entirety) describes affinity maturation by V_{H} and V_{L} domain shuffling. Random mutagenesis of CDR and/or framework residues is described by, for example, Barbas et al., Proc. Nat. Acad. Sci. U.S.A., 1994, 91:3809-3813; Schier et al., Gene, 1995, 169:147-155; Yelton et al., J. Immunol., 1995, 155:1994-2004; Jackson et al., J. Immunol., 1995, 154:3310-33199; and Hawkins et al, J. Mol. Biol., 1992, 226:889-896; each of which is incorporated by reference in its entirety.

“Fc effector functions” refer to those biological activities mediated by the Fc region of an antibody, which activities may vary depending on the antibody isotype. Examples of antibody effector functions include C1q binding to activate complement dependent cytotoxicity (CDC), Fc receptor binding to activate antibody-dependent cellular cytotoxicity (ADCC), and antibody dependent cellular phagocytosis (ADCP).

When used herein in the context of two or more antibodies, the term “competes with” or “cross-competes with” indicates that the two or more antibodies compete for binding to an antigen (e.g., TF). In one exemplary assay, TF is coated on a surface and contacted with a first TF antibody, after which a second TF antibody is added. In another exemplary assay, first a TF antibody is coated on a surface and contacted with TF, and then a second TF antibody is added. If the presence of the first TF antibody reduces binding of the second TF antibody, in either assay, then the antibodies compete with each other. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least 25%, at least 50%, at least 60%, at least 70%, at least 80%, at least 85%, at least 90%, or at least 95%. A skilled artisan can select the concentrations of the antibodies used in the competition assays based on the affinities of the antibodies for TF and the valency of the antibodies. The assays described in this definition are illustrative, and a skilled artisan can utilize any suitable assay to determine if antibodies compete with each other. Suitable assays are described, for example, in Cox et al., “Immunoassay Methods,” in Assay Guidance Manual[Internet], Updated Dec. 24, 2014 (www.ncbi.nlm.nih.gov/books/NBK92434/; accessed Sep. 29, 2015); Silman et al., Cytometry, 2001, 44:30-37; and Finco et al., J. Pharm. Biomed. Anal., 2011, 54:351-358; each of which is incorporated by reference in its entirety. As provided in Example 8, antibodies of group 25 and antibodies of group 43 compete with each other for binding to human TF, while antibodies from groups 1, 29, 39, and 54 do not compete for binding to human TF with antibodies of groups 25 and 43.

As used herein, an antibody that binds specifically to a human antigen is considered to bind the same antigen of mouse origin when a K_{D} value can be measured on a ForteBio Octet with the mouse antigen. An antibody that binds specifically to a human antigen is considered to be “cross-reactive” with the same antigen of mouse origin when the K_{D} value for the mouse antigen is no greater than 20 times the corresponding K_{D} value for the respective human antigen. For example, the antibody M1593 described in U.S. Pat. Nos. 8,722,044, 8,951,525, and 8,999,333, each of which is herein incorporated by reference for all purposes, the humanized 5G9 antibody described in Ngo et al., 2007, Int J Cancer, 120(6):1261-1267, incorporated by reference in its entirety, and chimeric ALT-836 antibody described in Hong et al, 2012, J Nucl Med, 53(11):1748-1754, incorporated by reference in its entirety, do not bind to mouse TF. As provided in Examples 1 and 2, TF antibodies from groups 25 and 43 bind to mouse TF, e.g., the TF antibodies 25G, 25G1, 25G9, and 43D8 are cross-reactive with mouse TF.

As used herein, an antibody that binds specifically to a human antigen is considered to be “cross-reactive” with the same antigen of cynomolgus monkey origin when the K_{D} value for the cynomolgus monkey antigen is no greater than 15 times the corresponding K_{D} value for the respective human antigen. As provided in Example 1, all tested antibodies from groups 1, 25, 29, 39, 43, and 54 are cross-reactive with cynomolgus monkey TF.

The term “epitope” means a portion of an antigen that is specifically bound by an antibody. Epitopes frequently include surface-accessible amino acid residues and/or sugar side chains and may have specific three dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to TF variants with different point-mutations, or to chimeric TF variants.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution of an amino acid with a chemically or functionally similar amino acid. Conservative substitution tables providing similar amino acids are well known in the art. By way of example, the groups of amino acids provided in Tables 2-4 are, in some embodiments, considered conservative substitutions for one another.

TABLE 2
Selected groups of amino acids that are considered conservative
substitutions for one another, in certain embodiments.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 3
Additional selected groups of amino acids that are considered conservative
substitutions for one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 4
Further selected groups of amino acids that are considered conservative
substitutions for one another, in certain embodiments.

| | |
|---|---|
| Group A | A and G |
| Group B | D and E |
| Group C | N and Q |
| Group D | R, K, and H |
| Group E | I, L, M, V |
| Group F | F Y and W |
| Group G | S and T |
| Group H | C and M |

Additional conservative substitutions may be found, for example, in Creighton, Proteins: Structures and Molecular Properties 2nd ed. (1993) W. H. Freeman & Co., New York, NY. An antibody generated by making one or more conservative substitutions of amino acid residues in a parent antibody is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which an exogenous nucleic acid has been introduced, and the progeny of such cells. Host cells include “transformants” (or “transformed cells”) and “transfectants” (or “transfected cells”), which each include the primary transformed or transfected cell and progeny derived therefrom. Such progeny may not be completely identical in nucleic acid content to a parent cell, and may contain mutations.

The term “treating” (and variations thereof such as “treat” or “treatment”) refers to clinical intervention in an attempt to alter the natural course of a disease or condition in a subject in need thereof. Treatment can be performed both for prophylaxis and during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “therapeutically effective amount” or “effective amount” refers to an amount of an antibody or pharmaceutical composition provided herein that, when administered to a subject, is effective to treat a disease or disorder.

As used herein, the term “subject” means a mammalian subject. Exemplary subjects include humans, monkeys, dogs, cats, mice, rats, cows, horses, camels, goats, rabbits, pigs and sheep. In certain embodiments, the subject is a human. In some embodiments the subject has a disease or condition that can be treated with an antibody provided herein. In some aspects, the disease or condition is a cancer. In some aspects, the disease or condition involves neovascularization or vascular inflammation. In certain aspects, the disease or condition involving neovascularization is age-related macular degeneration (AIMD), diabetic retinopathy, or cancer.

The term “package insert” is used to refer to instructions customarily included in commercial packages of therapeutic or diagnostic products (e.g., kits) that contain information about the indications, usage, dosage, administration, combination therapy, contraindications and/or warnings concerning the use of such therapeutic or diagnostic products.

A “chemotherapeutic agent” refers to a chemical compound useful in the treatment of cancer. Chemotherapeutic agents include “anti-hormonal agents” or “endocrine therapeutics” which act to regulate, reduce, block, or inhibit the effects of hormones that can promote the growth of cancer.

The term “cytostatic agent” refers to a compound or composition which arrests growth of a cell either in vitro or in vivo. In some embodiments, a cytostatic agent is an agent that reduces the percentage of cells in S phase. In some embodiments, a cytostatic agent reduces the percentage of cells in S phase by at least about 20%, at least about 40%, at least about 60%, or at least about 80%.

The term “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “agonize” refers to the activation of receptor signaling to induce a biological response associated with activation of the receptor. An “agonist” is an entity that binds to and agonizes a receptor.

The term “antagonize” refers to the inhibition of receptor signaling to inhibit a biological response associated with activation of the receptor. An “antagonist” is an entity that binds to and antagonizes a receptor.

### 2. TF Antibodies

### 2.1. TF Binding

Provided herein are isolated antibodies that specifically bind to TF. In some aspects, the TF is hTF (SEQ ID NO:809). In some aspects, the TF is cTF (SEQ ID NO:813). In some aspects, the TF is mTF (SEQ ID NO:817). In some aspects, the TF is rabbit TF (SEQ ID NO:832). In some aspects, the TF is pTF (SEQ ID NO:824). In some embodiments, the antibodies provided herein specifically bind to hTF (SEQ ID NO:809), cTF (SEQ ID NO:813), mTF (SEQ ID NO:817), rabbit TF (SEQ ID NO:832), and pTF (SEQ ID NO:824). In some embodiments, the antibodies provided herein specifically bind to hTF (SEQ ID NO:809), cTF (SEQ ID NO:813), mTF (SEQ ID NO:817), and pTF (SEQ ID NO:824). In some embodiments, the antibodies provided herein specifically bind to hTF (SEQ ID NO:809), cTF (SEQ ID NO:813), and mTF (SEQ ID NO:817). In some embodiments, the antibodies provided herein specifically bind to hTF (SEQ ID NO:809) and cTF (SEQ ID NO:813). In some embodiments, the antibodies provided herein do not bind mTF (SEQ ID NO:817). In some embodiments, the antibodies provided herein do not bind pTF (SEQ ID NO:824). In some embodiments, the antibodies provided herein do not bind rabbit TF (SEQ ID NO:832).

In various embodiments, the antibodies provided herein specifically bind to the extracellular domain of human TF (SEQ ID NO:810).

In some embodiments, the binding between an antibody provided herein and a variant TF extracellular domain comprising a mutation at amino acid residue 149 of the sequence shown in SEQ ID NO:810 is less than 50% of the binding between the antibody provided herein and the extracellular domain of TF of the sequence shown in SEQ ID NO:810, as determined by the median fluorescence intensity value of the antibody relative to an isotype control in a live cell staining assay. In some embodiments, the mutation at amino acid residue 149 of the sequence shown in SEQ ID NO:810 is K149N.

In some embodiments, the binding between an antibody provided herein and a variant TF extracellular domain comprising a mutation at amino acid residue 68 of the sequence shown in SEQ ID NO:810 is greater than 50% of the binding between the antibody provided herein and the extracellular domain of TF of the sequence shown in SEQ ID NO:810, as determined by the median fluorescence intensity value of the antibody relative to an isotype control in a live cell staining assay. In some embodiments, the mutation at amino acid residue 68 of the sequence shown in SEQ ID NO:810 is K68N.

In some embodiments, the binding between an antibody provided herein and a variant TF extracellular domain comprising mutations at amino acid residues 171 and 197 of the sequence shown in SEQ ID NO:810 is less than 50% of the binding between the antibody provided herein and the extracellular domain of TF of the sequence shown in SEQ ID NO:810, as determined by the median fluorescence intensity value of the antibody relative to an isotype control in a live cell staining assay. In some embodiments, the mutations at amino acid residues 171 and 197 of the sequence shown in SEQ ID NO:810 are N171H and T197K.

In some embodiments, the binding between an antibody provided herein and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody provided herein and the extracellular domain of TF of the sequence shown in SEQ ID NO:810, as determined by the median fluorescence intensity value of the antibody relative to an isotype control in a live cell staining assay.

In some embodiments, the binding between an antibody provided herein and a variant TF extracellular domain comprising a mutation at amino acid residue 149 of the sequence shown in SEQ ID NO:810 is less than 50% of the binding between the antibody provided herein and the extracellular domain of TF of the sequence shown in SEQ ID NO:810; the binding between an antibody provided herein and a variant TF extracellular domain comprising a mutation at amino acid residue 68 of the sequence shown in SEQ ID NO:810 is greater than 50% of the binding between the antibody provided herein and the extracellular domain of TF of the sequence shown in SEQ ID NO:810; the binding between an antibody provided herein and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810; the binding between an antibody provided herein and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810; the binding between an antibody provided herein and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810; the binding between an antibody provided herein and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810; and the binding between an antibody provided herein and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody provided herein and the extracellular domain of TF of the sequence shown in SEQ ID NO:810, as determined by the median fluorescence intensity value of the antibody relative to an isotype control in a live cell staining assay. In some embodiments, the mutation at amino acid residue 149 of the sequence shown in SEQ ID NO:810 is K149N; and the mutation at amino acid residue 68 of the sequence shown in SEQ ID NO:810 is K68N.

In some embodiments, the binding between an antibody provided herein and a variant TF extracellular domain comprising a mutation at amino acid residue 149 of the sequence shown in SEQ ID NO:810 is less than 50% of the binding between the antibody provided herein and the extracellular domain of TF of the sequence shown in SEQ ID NO:810; the binding between an antibody provided herein and a variant TF extracellular domain comprising a mutation at amino acid residue 68 of the sequence shown in SEQ ID NO:810 is greater than 50% of the binding between the antibody provided herein and the extracellular domain of TF of the sequence shown in SEQ ID NO:810; the binding between an antibody provided herein and a variant TF extracellular domain comprising mutations at amino acid residues 171 and 197 of the sequence shown in SEQ ID NO:810 is less than 50% of the binding between the antibody provided herein and the extracellular domain of TF of the sequence shown in SEQ ID NO:810; the binding between an antibody provided herein and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810; the binding between an antibody provided herein and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810; the binding between an antibody provided herein and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810; the binding between an antibody provided herein and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810; the binding between an antibody provided herein and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the antibody and the extracellular domain of TF of the sequence shown in SEQ ID NO:810; the binding between an antibody provided herein and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the antibody and the extracellular domain of TF of the sequence shown in SEQ ID NO:810; the binding between an antibody provided herein and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the antibody and the extracellular domain of TF of the sequence shown in SEQ ID NO:810; the binding between an antibody provided herein and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the antibody and the extracellular domain of TF of the sequence shown in SEQ ID NO:810; and the binding between an antibody provided herein and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody provided herein and the extracellular domain of TF of the sequence shown in SEQ ID NO:810, as determined by the median fluorescence intensity value of the antibody relative to an isotype control in a live cell staining assay. In some embodiments, the mutation at amino acid residue 149 of the sequence shown in SEQ ID NO:810 is K149N; the mutation at amino acid residue 68 of the sequence shown in SEQ ID NO:810 is K68N; and the mutations at amino acid residues 171 and 197 of the sequence shown in SEQ ID NO:810 are N171H and T197K.

In some embodiments, the antibodies provided herein are inert in inhibiting human thrombin generation as determined by thrombin generation assay (TGA) compared to a reference antibody M1593, wherein the reference antibody M1593 comprises a V_{H} sequence of SEQ ID NO:821 and a V_{L} sequence of SEQ ID NO:822.

In some embodiments, the antibodies provided herein do not inhibit human thrombin generation as determined by thrombin generation assay (TGA). In certain embodiments, the antibodies provided herein allow human thrombin generation as determined by thrombin generation assay (TGA).

In some embodiments, the antibodies provided herein bind human TF at a human TF binding site that is distinct from a human TF binding site bound by human FX. In certain embodiments, the antibodies provided herein do not interfere with the ability of TF:FVIIa to convert FX into FXa.

In some embodiments, the antibodies provided herein bind human TF at a human TF binding site that is distinct from a human TF binding site bound by human FVIIa. In certain embodiments, the antibodies provided herein do not compete for binding to human TF with human FVIIa.

In some embodiments, the antibodies provided herein bind to the extracellular domain of human TF, bind human TF at a human TF binding site that is distinct from a human TF binding site bound by human FVIIa, bind human TF at a human TF binding site that is distinct from a human TF binding site bound by human FX, and allow human thrombin generation as determined by thrombin generation assay (TGA).

In some embodiments, the antibodies provided herein bind to the extracellular domain of human TF, do not inhibit human thrombin generation as determined by thrombin generation assay (TGA), do not interfere with the ability of TF:FVIIa to convert FX into FXa, and do not compete for binding to human TF with human FVIIa.

In some embodiments, the antibodies provided herein bind to the extracellular domain of human TF at a human TF binding site that is distinct from a human TF binding site bound by human FVIIa, do not inhibit human thrombin generation as determined by thrombin generation assay (TGA), allow human thrombin generation as determined by thrombin generation assay (TGA), bind to human TF at a human TF binding site that is distinct from a human TF binding site bound by human FX, do not interfere with the ability of TF:FVIIa to convert FX into FXa, and do not compete for binding to human TF with human FVIIa.

In some embodiments, the antibodies provided herein inhibit FVIIa-dependent TF signaling.

In some embodiments, the antibodies provided herein reduce lesion size in a swine choroidal neovascularization (CNV) model.

In some embodiments, the antibodies provided herein bind to the extracellular domain of human TF at a human TF binding site that is distinct from a human TF binding site bound by human FVIIa, do not inhibit human thrombin generation as determined by thrombin generation assay (TGA), allow human thrombin generation as determined by thrombin generation assay (TGA), bind to human TF at a human TF binding site that is distinct from a human TF binding site bound by human FX, do not interfere with the ability of TF:FVIIa to convert FX into FXa, do not compete for binding to human TF with human FVIIa, and bind to cynomolgus and mouse TF.

In some embodiments, the antibodies provided herein bind to the extracellular domain of human TF at a human TF binding site that is distinct from a human TF binding site bound by human FVIIa, do not inhibit human thrombin generation as determined by thrombin generation assay (TGA), allow human thrombin generation as determined by thrombin generation assay (TGA), bind to human TF at a human TF binding site that is distinct from a human TF binding site bound by human FX, do not interfere with the ability of TF:FVIIa to convert FX into FXa, do not compete for binding to human TF with human FVIIa, bind to cynomolgus, mouse, and pig TF, and reduce lesion size in a swine choroidal neovascularization (CNV) model.

In some embodiments, the antibodies provided herein bind to the extracellular domain of human TF, inhibit FVIIa-dependent TF signaling, and bind to cynomolgus TF.

### 2.2. Sequences of TF Antibodies

### 2.2.1. V_{H} Domains

In some embodiments, an antibody provided herein comprises a V_{H} sequence selected from SEQ ID NOs: 37, 75, 113, 151, 189, 227, 265, 303, 341, 379, 417, 455, 493, 531, 569, 607, 645, 683, 721, and 759. In some embodiments, an antibody provided herein comprises a V_{H} sequence of SEQ ID NO:37. In some embodiments, an antibody provided herein comprises a V_{H} sequence of SEQ ID NO:75. In some embodiments, an antibody provided herein comprises a V_{H} sequence of SEQ ID NO:113. In some embodiments, an antibody provided herein comprises a V_{H} sequence of SEQ ID NO:151. In some embodiments, an antibody provided herein comprises a V_{H} sequence of SEQ ID NO:189. In some embodiments, an antibody provided herein comprises a V_{H} sequence of SEQ ID NO:836. In some embodiments, an antibody provided herein comprises a V_{H} sequence of SEQ ID NO:227. In some embodiments, an antibody provided herein comprises a V_{H} sequence of SEQ ID NO:265. In some embodiments, an antibody provided herein comprises a V_{H} sequence of SEQ ID NO:303. In some embodiments, an antibody provided herein comprises a V_{H} sequence of SEQ ID NO:341. In some embodiments, an antibody provided herein comprises a V_{H} sequence of SEQ ID NO:379. In some embodiments, an antibody provided herein comprises a V_{H} sequence of SEQ ID NO:417. In some embodiments, an antibody provided herein comprises a V_{H} sequence of SEQ ID NO:455. In some embodiments, an antibody provided herein comprises a V_{H} sequence of SEQ ID NO:493. In some embodiments, an antibody provided herein comprises a V_{H} sequence of SEQ ID NO:531. In some embodiments, an antibody provided herein comprises a V_{H} sequence of SEQ ID NO:569. In some embodiments, an antibody provided herein comprises a V_{H} sequence of SEQ ID NO:607. In some embodiments, an antibody provided herein comprises a V_{H} sequence of SEQ ID NO:645. In some embodiments, an antibody provided herein comprises a V_{H} sequence of SEQ ID NO:683. In some embodiments, an antibody provided herein comprises a V_{H} sequence of SEQ ID NO:721. In some embodiments, an antibody provided herein comprises a V_{H} sequence of SEQ ID NO:759.

In some embodiments, an antibody provided herein comprises a V_{H} sequence having at least about 50%, 60%, 70%, 80%, 90%, 95%, or 99% identity to an illustrative V_{H} sequence provided in SEQ ID NOs: 37, 75, 113, 151, 189, 227, 265, 303, 341, 379, 417, 455, 493, 531, 569, 607, 645, 683, 721, and 759. In some embodiments, an antibody provided herein comprises a V_{H} sequence provided in SEQ ID NOs: 37, 75, 113, 151, 189, 227, 265, 303, 341, 379, 417, 455, 493, 531, 569, 607, 645, 683, 721, and 759, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### 2.2.2. V_{L} Domains

In some embodiments, an antibody provided herein comprises a V_{L} sequence selected from SEQ ID NOs: 38, 76, 114, 152, 190, 228, 266, 304, 342, 380, 418, 456, 494, 532, 570, 608, 646, 684, 722, and 760. In some embodiments, an antibody provided herein comprises a V_{L} sequence of SEQ ID NO:38. In some embodiments, an antibody provided herein comprises a V_{L} sequence of SEQ ID NO:76. In some embodiments, an antibody provided herein comprises a V_{L} sequence of SEQ ID NO:114. In some embodiments, an antibody provided herein comprises a V_{L} sequence of SEQ ID NO:152. In some embodiments, an antibody provided herein comprises a V_{L} sequence of SEQ ID NO:190. In some embodiments, an antibody provided herein comprises a V_{L} sequence of SEQ ID NO:837. In some embodiments, an antibody provided herein comprises a V_{L} sequence of SEQ ID NO:228. In some embodiments, an antibody provided herein comprises a V_{L} sequence of SEQ ID NO:266. In some embodiments, an antibody provided herein comprises a V_{L} sequence of SEQ ID NO:304. In some embodiments, an antibody provided herein comprises a V_{L} sequence of SEQ ID NO:342. In some embodiments, an antibody provided herein comprises a V_{L} sequence of SEQ ID NO:380. In some embodiments, an antibody provided herein comprises a V_{L} sequence of SEQ ID NO:418. In some embodiments, an antibody provided herein comprises a V_{L} sequence of SEQ ID NO:456. In some embodiments, an antibody provided herein comprises a V_{L} sequence of SEQ ID NO:494. In some embodiments, an antibody provided herein comprises a V_{L} sequence of SEQ ID NO:532. In some embodiments, an antibody provided herein comprises a V_{L} sequence of SEQ ID NO:570. In some embodiments, an antibody provided herein comprises a V_{L} sequence of SEQ ID NO:608. In some embodiments, an antibody provided herein comprises a V_{L} sequence of SEQ ID NO:646. In some embodiments, an antibody provided herein comprises a V_{L} sequence of SEQ ID NO:684. In some embodiments, an antibody provided herein comprises a V_{L} sequence of SEQ ID NO:722. In some embodiments, an antibody provided herein comprises a V_{L} sequence of SEQ ID NO:760.

In some embodiments, an antibody provided herein comprises a V_{L} sequence having at least about 50%, 60%, 70%, 80%, 90%, 95%, or 99% identity to an illustrative V_{L} sequence provided in SEQ ID NOs: 38, 76, 114, 152, 190, 228, 266, 304, 342, 380, 418, 456, 494, 532, 570, 608, 646, 684, 722, and 760. In some embodiments, an antibody provided herein comprises a V_{L} sequence provided in SEQ ID NOs: 38, 76, 114, 152, 190, 228, 266, 304, 342, 380, 418, 456, 494, 532, 570, 608, 646, 684, 722, and 760, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

2.2.3. V_{H}-V_{L} Combinations

In some embodiments, an antibody provided herein comprises a V_{H} sequence selected from SEQ ID NOs: 37, 75, 113, 151, 189, 227, 265, 303, 341, 379, 417, 455, 493, 531, 569, 607, 645, 683, 721, and 759 and a V_{L} sequence selected from SEQ ID NOs: 38, 76, 114, 152, 190, 228, 266, 304, 342, 380, 418, 456, 494, 532, 570, 608, 646, 684, 722, and 760.

In some embodiments, an antibody provided herein comprises a V_{H} sequence of SEQ ID NO:37 and a V_{L} sequence of SEQ ID NO:38. In some embodiments, an antibody provided herein comprises a V_{H} sequence of SEQ ID NO:75 and a V_{L} sequence of SEQ ID NO:76. In some embodiments, an antibody provided herein comprises a V_{H} sequence of SEQ ID NO:113 and a V_{L} sequence of SEQ ID NO:114. In some embodiments, an antibody provided herein comprises a V_{H} sequence of SEQ ID NO:151 and a V_{L} sequence of SEQ ID NO:152. In some embodiments, an antibody provided herein comprises a V_{H} sequence of SEQ ID NO:189 and a V_{L} sequence of SEQ ID NO:190. In some embodiments, an antibody provided herein comprises a V_{H} sequence of SEQ ID NO:836 and a V_{L} sequence of SEQ ID NO:837. In some embodiments, an antibody provided herein comprises a V_{H} sequence of SEQ ID NO:227 and a V_{L} sequence of SEQ ID NO:228. In some embodiments, an antibody provided herein comprises a V_{H} sequence of SEQ ID NO:265 and a V_{L} sequence of SEQ ID NO:266. In some embodiments, an antibody provided herein comprises a V_{H} sequence of SEQ ID NO:303 and a V_{L} sequence of SEQ ID NO:304. In some embodiments, an antibody provided herein comprises a V_{H} sequence of SEQ ID NO:341 and a V_{L} sequence of SEQ ID NO:342. In some embodiments, an antibody provided herein comprises a V_{H} sequence of SEQ ID NO:379 and a V_{L} sequence of SEQ ID NO:380. In some embodiments, an antibody provided herein comprises a V_{H} sequence of SEQ ID NO:417 and a V_{L} sequence of SEQ ID NO:418. In some embodiments, an antibody provided herein comprises a V_{H} sequence of SEQ ID NO:455 and a V_{L} sequence of SEQ ID NO:456. In some embodiments, an antibody provided herein comprises a V_{H} sequence of SEQ ID NO:493 and a V_{L} sequence of SEQ ID NO:494. In some embodiments, an antibody provided herein comprises a V_{H} sequence of SEQ ID NO:531 and a V_{L} sequence of SEQ ID NO:532. In some embodiments, an antibody provided herein comprises a V_{H} sequence of SEQ ID NO:569 and a V_{L} sequence of SEQ ID NO:570. In some embodiments, an antibody provided herein comprises a V_{H} sequence of SEQ ID NO:607 and a V_{L} sequence of SEQ ID NO:608. In some embodiments, an antibody provided herein comprises a V_{H} sequence of SEQ ID NO:645 and a V_{L} sequence of SEQ ID NO:646. In some embodiments, an antibody provided herein comprises a V_{H} sequence of SEQ ID NO:683 and a V_{L} sequence of SEQ ID NO:684. In some embodiments, an antibody provided herein comprises a V_{H} sequence of SEQ ID NO:721 and a V_{L} sequence of SEQ ID NO:722. In some embodiments, an antibody provided herein comprises a V_{H} sequence of SEQ ID NO:759 and a V_{L} sequence of SEQ ID NO:760.

In some embodiments, an antibody provided herein comprises a V_{H} sequence having at least about 50%, 60%, 70%, 80%, 90%, 95%, or 99% identity to an illustrative V_{H} sequence provided in SEQ ID NOs: 37, 75, 113, 151, 189, 227, 265, 303, 341, 379, 417, 455, 493, 531, 569, 607, 645, 683, 721, and 759, and a V_{L} sequence having at least about 50%, 60%, 70%, 80%, 90%, 95%, or 99% identity to an illustrative V_{L} sequence provided in SEQ ID NOs: 38, 76, 114, 152, 190, 228, 266, 304, 342, 380, 418, 456, 494, 532, 570, 608, 646, 684, 722, and 760. In some embodiments, an antibody provided herein comprises a V_{H} sequence provided in SEQ ID NOs: 37, 75, 113, 151, 189, 227, 265, 303, 341, 379, 417, 455, 493, 531, 569, 607, 645, 683, 721, and 759, with up to 1, 2, 3, 4, 5, 6, 7, 8, 9, 10, 11, 12, 13, 14, 15, 16, 17, 18, 19, 20, 21, 22, 23, 24, or 25 amino acid substitutions, and a V_{L} sequence provided in SEQ ID NOs: 38, 76, 114, 152, 190, 228, 266, 304, 342, 380, 418, 456, 494, 532, 570, 608, 646, 684, 722, and 760, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### 2.2.4. CDRs

In some embodiments, an antibody provided herein comprises one to three CDRs of a V_{H} domain selected from SEQ ID NOs: 37, 75, 113, 151, 189, 227, 265, 303, 341, 379, 417, 455, 493, 531, 569, 607, 645, 683, 721, and 759. In some embodiments, an antibody provided herein comprises two to three CDRs of a V_{H} domain selected from SEQ ID NOs: 37, 75, 113, 151, 189, 227, 265, 303, 341, 379, 417, 455, 493, 531, 569, 607, 645, 683, 721, and 759. In some embodiments, an antibody provided herein comprises three CDRs of a V_{H} domain selected from SEQ ID NOs: 37, 75, 113, 151, 189, 227, 265, 303, 341, 379, 417, 455, 493, 531, 569, 607, 645, 683, 721, and 759. In some aspects, the CDRs are Exemplary CDRs. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, the CDRs are CDRs having at least about 50%, 75%, 80%, 85%, 90%, or 95% identity with a CDR-H1, CDR-H2, or CDR-H3 of SEQ ID NOs: 37, 75, 113, 151, 189, 227, 265, 303, 341, 379, 417, 455, 493, 531, 569, 607, 645, 683, 721, and 759. In some embodiments, the CDR-H1 is a CDR-H1 of a V_{H} domain selected from SEQ ID NOs: 37, 75, 113, 151, 189, 227, 265, 303, 341, 379, 417, 455, 493, 531, 569, 607, 645, 683, 721, and 759, with up to 1, 2, 3, 4, or 5 amino acid substitutions. In some embodiments, the CDR-H2 is a CDR-H2 of a V_{H} domain selected from SEQ ID NOs: 37, 75, 113, 151, 189, 227, 265, 303, 341, 379, 417, 455, 493, 531, 569, 607, 645, 683, 721, and 759, with up to 1, 2, 3, 4, 5, 6, 7, or 8 amino acid substitutions. In some embodiments, the CDR-H3 is a CDR-H3 of a V_{H} domain selected from SEQ ID NOs: 37, 75, 113, 151, 189, 227, 265, 303, 341, 379, 417, 455, 493, 531, 569, 607, 645, 683, 721, and 759, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises one to three CDRs of a V_{L} domain selected from SEQ ID NOs: 38, 76, 114, 152, 190, 228, 266, 304, 342, 380, 418, 456, 494, 532, 570, 608, 646, 684, 722, and 760. In some embodiments, an antibody provided herein comprises two to three CDRs of a V_{L} domain selected from SEQ ID NOs: 38, 76, 114, 152, 190, 228, 266, 304, 342, 380, 418, 456, 494, 532, 570, 608, 646, 684, 722, and 760. In some embodiments, an antibody provided herein comprises three CDRs of a V_{L} domain selected from SEQ ID NOs: 38, 76, 114, 152, 190, 228, 266, 304, 342, 380, 418, 456, 494, 532, 570, 608, 646, 684, 722, and 760. In some aspects, the CDRs are Exemplary CDRs. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, the CDRs are CDRs having at least about 50%, 75%, 80%, 85%, 90%, or 95% identity with a CDR-L1, CDR-L2, or CDR-L3 of SEQ ID NOs: 38, 76, 114, 152, 190, 228, 266, 304, 342, 380, 418, 456, 494, 532, 570, 608, 646, 684, 722, and 760. In some embodiments, the CDR-L1 is a CDR-L1 of a V_{L} domain selected from SEQ ID NOs: 38, 76, 114, 152, 190, 228, 266, 304, 342, 380, 418, 456, 494, 532, 570, 608, 646, 684, 722, and 760, with up to 1, 2, 3, 4, or 5 amino acid substitutions. In some embodiments, the CDR-L2 is a CDR-L2 of a V_{L} domain selected from SEQ ID NOs: 38, 76, 114, 152, 190, 228, 266, 304, 342, 380, 418, 456, 494, 532, 570, 608, 646, 684, 722, and 760, with up to 1, 2, 3, 4, 5, 6, 7, or 8 amino acid substitutions. In some embodiments, the CDR-L3 is a CDR-L3 of a V_{L} domain selected from SEQ ID NOs: 38, 76, 114, 152, 190, 228, 266, 304, 342, 380, 418, 456, 494, 532, 570, 608, 646, 684, 722, and 760, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises one to three CDRs of a V_{H} domain selected from SEQ ID NOs: 37, 75, 113, 151, 189, 227, 265, 303, 341, 379, 417, 455, 493, 531, 569, 607, 645, 683, 721, and 759 and one to three CDRs of a V_{L} domain selected from SEQ ID NOs: 38, 76, 114, 152, 190, 228, 266, 304, 342, 380, 418, 456, 494, 532, 570, 608, 646, 684, 722, and 760. In some embodiments, an antibody provided herein comprises two to three CDRs of a V_{H} domain selected from SEQ ID NOs: 37, 75, 113, 151, 189, 227, 265, 303, 341, 379, 417, 455, 493, 531, 569, 607, 645, 683, 721, and 759 and two to three CDRs of a V_{L} domain selected from SEQ ID NOs: 38, 76, 114, 152, 190, 228, 266, 304, 342, 380, 418, 456, 494, 532, 570, 608, 646, 684, 722, and 760. In some embodiments, an antibody provided herein comprises three CDRs of a V_{H} domain selected from SEQ ID NOs: 37, 75, 113, 151, 189, 227, 265, 303, 341, 379, 417, 455, 493, 531, 569, 607, 645, 683, 721, and 759 and three CDRs of a V_{L} domain selected from SEQ ID NOs: 38, 76, 114, 152, 190, 228, 266, 304, 342, 380, 418, 456, 494, 532, 570, 608, 646, 684, 722, and 760. In some aspects, the CDRs are Exemplary CDRs. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, the CDRs are CDRs having at least about 50%, 75%, 80%, 85%, 90%, or 95% identity with a CDR-H1, CDR-H2, or CDR-H3 of SEQ ID NOs: 37, 75, 113, 151, 189, 227, 265, 303, 341, 379, 417, 455, 493, 531, 569, 607, 645, 683, 721, and 759 and at least about 50%, 75%, 80%, 85%, 90%, or 95% identity with a CDR-L1, CDR-L2, or CDR-L3 of SEQ ID NOs: 38, 76, 114, 152, 190, 228, 266, 304, 342, 380, 418, 456, 494, 532, 570, 608, 646, 684, 722, and 760. In some embodiments, the CDR-H1 is a CDR-H1 of a V_{H} domain selected from SEQ ID NOs: 37, 75, 113, 151, 189, 227, 265, 303, 341, 379, 417, 455, 493, 531, 569, 607, 645, 683, 721, and 759, with up to 1, 2, 3, 4, or 5 amino acid substitutions; the CDR-H2 is a CDR-H2 of a V_{H} domain selected from SEQ ID NOs: 37, 75, 113, 151, 189, 227, 265, 303, 341, 379, 417, 455, 493, 531, 569, 607, 645, 683, 721, and 759, with up to 1, 2, 3, 4, 5, 6, 7, or 8 amino acid substitutions; the CDR-H3 is a CDR-H3 of a V_{H} domain selected from SEQ ID NOs: 37, 75, 113, 151, 189, 227, 265, 303, 341, 379, 417, 455, 493, 531, 569, 607, 645, 683, 721, and 759, with up to 1, 2, 3, 4, 5, 6, 7, or 8 amino acid substitutions; the CDR-L1 is a CDR-L1 of a V_{L} domain selected from SEQ ID NOs: 38, 76, 114, 152, 190, 228, 266, 304, 342, 380, 418, 456, 494, 532, 570, 608, 646, 684, 722, and 760, with up to 1, 2, 3, 4, 5, or 6 amino acid substitutions; the CDR-L2 is a CDR-L2 of a V_{L} domain selected from SEQ ID NOs: 38, 76, 114, 152, 190, 228, 266, 304, 342, 380, 418, 456, 494, 532, 570, 608, 646, 684, 722, and 760, with up to 1, 2, 3, or 4 amino acid substitutions; and the CDR-L3 is a CDR-L3 of a V_{L} domain selected from SEQ ID NOs: 38, 76, 114, 152, 190, 228, 266, 304, 342, 380, 418, 456, 494, 532, 570, 608, 646, 684, 722, and 760, with up to 1, 2, 3, 4, or 5 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 selected from SEQ ID NOs: 3, 41, 79, 117, 155, 193, 231, 269, 307, 345, 383, 421, 459, 497, 535, 573, 611, 649, 687, and 725, as determined by the Exemplary numbering system. In some aspects, the CDR-H3 has at least about 50%, 75%, 80%, 85%, 90%, or 95% identity with a CDR-H3 of SEQ ID NOs: 3, 41, 79, 117, 155, 193, 231, 269, 307, 345, 383, 421, 459, 497, 535, 573, 611, 649, 687, and 725. In some embodiments, the CDR-H3 is a CDR-H3 selected from SEQ ID NOs: 3, 41, 79, 117, 155, 193, 231, 269, 307, 345, 383, 421, 459, 497, 535, 573, 611, 649, 687, and 725, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H2 selected from SEQ ID NOs: 2, 40, 78, 116, 154, 192, 230, 268, 306, 344, 382, 420, 458, 496, 534, 572, 610, 648, 686, and 724, as determined by the Exemplary numbering system. In some aspects, the CDR-H2 has at least about 50%, 75%, 80%, 85%, 90%, or 95% identity with a CDR-H2 of SEQ ID NOs: 2, 40, 78, 116, 154, 192, 230, 268, 306, 344, 382, 420, 458, 496, 534, 572, 610, 648, 686, and 724. In some embodiments, the CDR-H2 is a CDR-H2 selected from SEQ ID NOs: 2, 40, 78, 116, 154, 192, 230, 268, 306, 344, 382, 420, 458, 496, 534, 572, 610, 648, 686, and 724, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H1 selected from SEQ ID NOs: 1, 39, 77, 115, 153, 191, 229, 267, 305, 343, 381, 419, 457, 495, 533, 571, 609, 647, 685, and 723, as determined by the Exemplary numbering system. In some aspects, the CDR-H1 has at least about 50%, 75%, 80%, 85%, 90%, or 95% identity with a CDR-H1 of SEQ ID NOs: 1, 39, 77, 115, 153, 191, 229, 267, 305, 343, 381, 419, 457, 495, 533, 571, 609, 647, 685, and 723. In some embodiments, the CDR-H1 is a CDR-H1 selected from SEQ ID NOs: 1, 39, 77, 115, 153, 191, 229, 267, 305, 343, 381, 419, 457, 495, 533, 571, 609, 647, 685, and 723, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 selected from SEQ ID NOs: 3, 41, 79, 117, 155, 193, 231, 269, 307, 345, 383, 421, 459, 497, 535, 573, 611, 649, 687, and 725 and a CDR-H2 selected from SEQ ID NOs: 2, 40, 78, 116, 154, 192, 230, 268, 306, 344, 382, 420, 458, 496, 534, 572, 610, 648, 686, and 724. In some embodiments, an antibody provided herein comprises a CDR-H3 selected from SEQ ID NOs: 3, 41, 79, 117, 155, 193, 231, 269, 307, 345, 383, 421, 459, 497, 535, 573, 611, 649, 687, and 725, a CDR-H2 selected from SEQ ID NOs: 2, 40, 78, 116, 154, 192, 230, 268, 306, 344, 382, 420, 458, 496, 534, 572, 610, 648, 686, and 724, and a CDR-H1 selected from SEQ ID NOs: 1, 39, 77, 115, 153, 191, 229, 267, 305, 343, 381, 419, 457, 495, 533, 571, 609, 647, 685, and 723. In some embodiments, the CDR-H3 has at least about 50%, 75%, 80%, 85%, 90%, or 95% identity with a CDR-H3 of SEQ ID NOs: 3, 41, 79, 117, 155, 193, 231, 269, 307, 345, 383, 421, 459, 497, 535, 573, 611, 649, 687, and 725, the CDR-H2 has at least about 50%, 75%, 80%, 85%, 90%, or 95% identity with a CDR-H2 of SEQ ID NOs: 2, 40, 78, 116, 154, 192, 230, 268, 306, 344, 382, 420, 458, 496, 534, 572, 610, 648, 686, and 724, and the CDR-H1 has at least about 50%, 75%, 80%, 85%, 90%, or 95% identity with a CDR-H1 of SEQ ID NOs: 1, 39, 77, 115, 153, 191, 229, 267, 305, 343, 381, 419, 457, 495, 533, 571, 609, 647, 685, and 723. In some embodiments, the CDR-H3 is a CDR-H3 selected from SEQ ID NOs: 3, 41, 79, 117, 155, 193, 231, 269, 307, 345, 383, 421, 459, 497, 535, 573, 611, 649, 687, and 725, with up to 1, 2, 3, 4, 5, 6, 7, or 8 amino acid substitutions; the CDR-H2 is a CDR-H2 selected from SEQ ID NOs: 2, 40, 78, 116, 154, 192, 230, 268, 306, 344, 382, 420, 458, 496, 534, 572, 610, 648, 686, and 724, with up to 1, 2, 3, 4, 5, 6, 7, or 8 amino acid substitutions; and the CDR-H1 is a CDR-H1 selected from SEQ ID NOs: 1, 39, 77, 115, 153, 191, 229, 267, 305, 343, 381, 419, 457, 495, 533, 571, 609, 647, 685, and 723, with up to 1, 2, 3, 4, or 5 amino acid substitutions. In some aspects, the amino acid substitutions are conservative amino acid substitutions. In some embodiments, the antibody described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-L3 selected from SEQ ID NOs: 6, 44, 82, 120, 158, 196, 234, 272, 310, 348, 386, 424, 462, 500, 538, 576, 614, 652, 690, and 728, as determined by the Exemplary numbering system. In some aspects, the CDR-L3 has at least about 50%, 75%, 80%, 85%, 90%, or 95% identity with a CDR-L3 of SEQ ID NOs: 6, 44, 82, 120, 158, 196, 234, 272, 310, 348, 386, 424, 462, 500, 538, 576, 614, 652, 690, and 728. In some embodiments, the CDR-L3 is a CDR-L3 selected from SEQ ID NOs: 6, 44, 82, 120, 158, 196, 234, 272, 310, 348, 386, 424, 462, 500, 538, 576, 614, 652, 690, and 728, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-L2 selected from SEQ ID NOs: 5, 43, 81, 119, 157, 195, 233, 271, 309, 347, 385, 423, 461, 499, 537, 575, 613, 651, 689, and 727, as determined by the Exemplary numbering system. In some aspects, the CDR-L2 has at least about 50%, 75%, 80%, 85%, 90%, or 95% identity with a CDR-L2 of SEQ ID NOs: 5, 43, 81, 119, 157, 195, 233, 271, 309, 347, 385, 423, 461, 499, 537, 575, 613, 651, 689, and 727. In some embodiments, the CDR-L2 is a CDR-L2 selected from SEQ ID NOs: 5, 43, 81, 119, 157, 195, 233, 271, 309, 347, 385, 423, 461, 499, 537, 575, 613, 651, 689, and 727, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-L1 selected from SEQ ID NOs: 4, 42, 80, 118, 156, 194, 232, 270, 308, 346, 384, 422, 460, 498, 536, 574, 612, 650, 688, and 726, as determined by the Exemplary numbering system. In some aspects, the CDR-L1 has at least about 50%, 75%, 80%, 85%, 90%, or 95% identity with a CDR-L1 of SEQ ID NOs: 4, 42, 80, 118, 156, 194, 232, 270, 308, 346, 384, 422, 460, 498, 536, 574, 612, 650, 688, and 726. In some embodiments, the CDR-L1 is a CDR-L1 selected from SEQ ID NOs: 4, 42, 80, 118, 156, 194, 232, 270, 308, 346, 384, 422, 460, 498, 536, 574, 612, 650, 688, and 726, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-L3 selected from SEQ ID NOs: 6, 44, 82, 120, 158, 196, 234, 272, 310, 348, 386, 424, 462, 500, 538, 576, 614, 652, 690, and 728 and a CDR-L2 selected from SEQ ID NOs: 5, 43, 81, 119, 157, 195, 233, 271, 309, 347, 385, 423, 461, 499, 537, 575, 613, 651, 689, and 727. In some embodiments, an antibody provided herein comprises a CDR-L3 selected from SEQ ID NOs: 6, 44, 82, 120, 158, 196, 234, 272, 310, 348, 386, 424, 462, 500, 538, 576, 614, 652, 690, and 728, a CDR-L2 selected from SEQ ID NOs: 5, 43, 81, 119, 157, 195, 233, 271, 309, 347, 385, 423, 461, 499, 537, 575, 613, 651, 689, and 727, and a CDR-L1 selected from SEQ ID NOs: 4, 42, 80, 118, 156, 194, 232, 270, 308, 346, 384, 422, 460, 498, 536, 574, 612, 650, 688, and 726. In some embodiments, the CDR-L3 has at least about 50%, 75%, 80%, 85%, 90%, or 95% identity with a CDR-L3 of SEQ ID NOs: 6, 44, 82, 120, 158, 196, 234, 272, 310, 348, 386, 424, 462, 500, 538, 576, 614, 652, 690, and 728, the CDR-L2 has at least about 50%, 75%, 80%, 85%, 90%, or 95% identity with a CDR-L2 of SEQ ID NOs: 5, 43, 81, 119, 157, 195, 233, 271, 309, 347, 385, 423, 461, 499, 537, 575, 613, 651, 689, and 727, and the CDR-L1 has at least about 50%, 75%, 80%, 85%, 90%, or 95% identity with a CDR-L1 of SEQ ID NOs: 4, 42, 80, 118, 156, 194, 232, 270, 308, 346, 384, 422, 460, 498, 536, 574, 612, 650, 688, and 726. In some embodiments, the CDR-L3 is a CDR-L3 selected from SEQ ID NOs: 6, 44, 82, 120, 158, 196, 234, 272, 310, 348, 386, 424, 462, 500, 538, 576, 614, 652, 690, and 728, with up to 1, 2, 3, 4, or 5 amino acid substitutions; the CDR-L2 is a CDR-L2 selected from SEQ ID NOs: 5, 43, 81, 119, 157, 195, 233, 271, 309, 347, 385, 423, 461, 499, 537, 575, 613, 651, 689, and 727, with up to 1, 2, 3, or 4 amino acid substitutions; and the CDR-L1 is a CDR-L1 selected from SEQ ID NOs: 4, 42, 80, 118, 156, 194, 232, 270, 308, 346, 384, 422, 460, 498, 536, 574, 612, 650, 688, and 726, with up to 1, 2, 3, 4, 5, or 6 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 selected from SEQ ID NOs: 3, 41, 79, 117, 155, 193, 231, 269, 307, 345, 383, 421, 459, 497, 535, 573, 611, 649, 687, and 725, a CDR-H2 selected from SEQ ID NOs: 2, 40, 78, 116, 154, 192, 230, 268, 306, 344, 382, 420, 458, 496, 534, 572, 610, 648, 686, and 724, a CDR-H1 selected from SEQ ID NOs: 1, 39, 77, 115, 153, 191, 229, 267, 305, 343, 381, 419, 457, 495, 533, 571, 609, 647, 685, and 723, a CDR-L3 selected from SEQ ID NOs: 6, 44, 82, 120, 158, 196, 234, 272, 310, 348, 386, 424, 462, 500, 538, 576, 614, 652, 690, and 728, a CDR-L2 selected from SEQ ID NOs: 5, 43, 81, 119, 157, 195, 233, 271, 309, 347, 385, 423, 461, 499, 537, 575, 613, 651, 689, and 727, and a CDR-L1 selected from SEQ ID NOs: 4, 42, 80, 118, 156, 194, 232, 270, 308, 346, 384, 422, 460, 498, 536, 574, 612, 650, 688, and 726. In some embodiments, the CDR-H3 has at least about 50%, 75%, 80%, 85%, 90%, or 95% identity with a CDR-H3 of SEQ ID NOs: 3, 41, 79, 117, 155, 193, 231, 269, 307, 345, 383, 421, 459, 497, 535, 573, 611, 649, 687, and 725, the CDR-H2 has at least about 50%, 75%, 80%, 85%, 90%, or 95% identity with a CDR-H2 of SEQ ID NOs: 2, 40, 78, 116, 154, 192, 230, 268, 306, 344, 382, 420, 458, 496, 534, 572, 610, 648, 686, and 724, the CDR-H1 has at least about 50%, 75%, 80%, 85%, 90%, or 95% identity with a CDR-H1 of SEQ ID NOs: 1, 39, 77, 115, 153, 191, 229, 267, 305, 343, 381, 419, 457, 495, 533, 571, 609, 647, 685, and 723, the CDR-L3 has at least about 50%, 75%, 80%, 85%, 90%, or 95% identity with a CDR-L3 of SEQ ID NOs: 6, 44, 82, 120, 158, 196, 234, 272, 310, 348, 386, 424, 462, 500, 538, 576, 614, 652, 690, and 728, the CDR-L2 has at least about 50%, 75%, 80%, 85%, 90%, or 95% identity with a CDR-L2 of SEQ ID NOs: 5, 43, 81, 119, 157, 195, 233, 271, 309, 347, 385, 423, 461, 499, 537, 575, 613, 651, 689, and 727, and the CDR-L1 has at least about 50%, 75%, 80%, 85%, 90%, or 95% identity with a CDR-L1 of SEQ ID NOs: 4, 42, 80, 118, 156, 194, 232, 270, 308, 346, 384, 422, 460, 498, 536, 574, 612, 650, 688, and 726. In some embodiments, the CDR-H3 is a CDR-H3 selected from SEQ ID NOs: 3, 41, 79, 117, 155, 193, 231, 269, 307, 345, 383, 421, 459, 497, 535, 573, 611, 649, 687, and 725, with up to 1, 2, 3, 4, 5, 6, 7, or 8 amino acid substitutions; the CDR-H2 is a CDR-H2 selected from SEQ ID NOs: 2, 40, 78, 116, 154, 192, 230, 268, 306, 344, 382, 420, 458, 496, 534, 572, 610, 648, 686, and 724, with up to 1, 2, 3, 4, 5, 6, 7, or 8 amino acid substitutions; the CDR-H1 is a CDR-H1 selected from SEQ ID NOs: 1, 39, 77, 115, 153, 191, 229, 267, 305, 343, 381, 419, 457, 495, 533, 571, 609, 647, 685, and 723, with up to 1, 2, 3, 4, or 5 amino acid substitutions; the CDR-L3 is a CDR-L3 selected from SEQ ID NOs: 6, 44, 82, 120, 158, 196, 234, 272, 310, 348, 386, 424, 462, 500, 538, 576, 614, 652, 690, and 728, with up to 1, 2, 3, 4, or 5 amino acid substitutions; the CDR-L2 is a CDR-L2 selected from SEQ ID NOs: 5, 43, 81, 119, 157, 195, 233, 271, 309, 347, 385, 423, 461, 499, 537, 575, 613, 651, 689, and 727, with up to 1, 2, 3, or 4 amino acid substitutions; and the CDR-L1 is a CDR-L1 selected from SEQ ID NOs: 4, 42, 80, 118, 156, 194, 232, 270, 308, 346, 384, 422, 460, 498, 536, 574, 612, 650, 688, and 726, with up to 1, 2, 3, 4, 5, or 6 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H1 of SEQ ID NO:1, a CDR-H2 of SEQ ID NO:2, a CDR-H3 of SEQ ID NO:3, a CDR-L1 of SEQ ID NO:4, a CDR-L2 of SEQ ID NO:5, and a CDR-L1 of SEQ ID NO:6, as determined by the Exemplary numbering system.

In some embodiments, an antibody provided herein comprises a CDR-H1 of SEQ ID NO:39, a CDR-H2 of SEQ ID NO:40, a CDR-H3 of SEQ ID NO:41, a CDR-L1 of SEQ ID NO:42, a CDR-L2 of SEQ ID NO:43, and a CDR-L1 of SEQ ID NO:44, as determined by the Exemplary numbering system.

In some embodiments, an antibody provided herein comprises a CDR-H1 of SEQ ID NO:77, a CDR-H2 of SEQ ID NO:78, a CDR-H3 of SEQ ID NO:79, a CDR-L1 of SEQ ID NO:80, a CDR-L2 of SEQ ID NO:81, and a CDR-L1 of SEQ ID NO:82, as determined by the Exemplary numbering system.

In some embodiments, an antibody provided herein comprises a CDR-H1 of SEQ ID NO: 115, a CDR-H2 of SEQ ID NO: 116, a CDR-H3 of SEQ ID NO: 117, a CDR-L1 of SEQ ID NO: 118, a CDR-L2 of SEQ ID NO: 119, and a CDR-L1 of SEQ ID NO:120, as determined by the Exemplary numbering system.

In some embodiments, an antibody provided herein comprises a CDR-H1 of SEQ ID NO:153, a CDR-H2 of SEQ ID NO:154, a CDR-H3 of SEQ ID NO:155, a CDR-L1 of SEQ ID NO:156, a CDR-L2 of SEQ ID NO:157, and a CDR-L1 of SEQ ID NO:158, as determined by the Exemplary numbering system.

In some embodiments, an antibody provided herein comprises a CDR-H1 of SEQ ID NO:884, a CDR-H2 of SEQ ID NO:885, a CDR-H3 of SEQ ID NO:886, a CDR-L1 of SEQ ID NO:887, a CDR-L2 of SEQ ID NO:888, and a CDR-L1 of SEQ ID NO:889, as determined by the Exemplary numbering system.

In some embodiments, an antibody provided herein comprises a CDR-H1 of SEQ ID NO:191, a CDR-H2 of SEQ ID NO:192, a CDR-H3 of SEQ ID NO:193, a CDR-L1 of SEQ ID NO:194, a CDR-L2 of SEQ ID NO:195, and a CDR-L1 of SEQ ID NO:196, as determined by the Exemplary numbering system.

In some embodiments, an antibody provided herein comprises a CDR-H1 of SEQ ID NO:229, a CDR-H2 of SEQ ID NO:230, a CDR-H3 of SEQ ID NO:231, a CDR-L1 of SEQ ID NO:232, a CDR-L2 of SEQ ID NO:233, and a CDR-L1 of SEQ ID NO:234, as determined by the Exemplary numbering system.

In some embodiments, an antibody provided herein comprises a CDR-H1 of SEQ ID NO:267, a CDR-H2 of SEQ ID NO:268, a CDR-H3 of SEQ ID NO:269, a CDR-L1 of SEQ ID NO:270, a CDR-L2 of SEQ ID NO:271, and a CDR-L1 of SEQ ID NO:272, as determined by the Exemplary numbering system.

In some embodiments, an antibody provided herein comprises a CDR-H1 of SEQ ID NO:305, a CDR-H2 of SEQ ID NO:306, a CDR-H3 of SEQ ID NO:307, a CDR-L1 of SEQ ID NO:308, a CDR-L2 of SEQ ID NO:309, and a CDR-L1 of SEQ ID NO:310, as determined by the Exemplary numbering system.

In some embodiments, an antibody provided herein comprises a CDR-H1 of SEQ ID NO:343, a CDR-H2 of SEQ ID NO:344, a CDR-H3 of SEQ ID NO:345, a CDR-L1 of SEQ ID NO:346, a CDR-L2 of SEQ ID NO:347, and a CDR-L1 of SEQ ID NO:348, as determined by the Exemplary numbering system.

In some embodiments, an antibody provided herein comprises a CDR-H1 of SEQ ID NO:381, a CDR-H2 of SEQ ID NO:382, a CDR-H3 of SEQ ID NO:383, a CDR-L1 of SEQ ID NO:384, a CDR-L2 of SEQ ID NO:385, and a CDR-L1 of SEQ ID NO:386, as determined by the Exemplary numbering system.

In some embodiments, an antibody provided herein comprises a CDR-H1 of SEQ ID NO:419, a CDR-H2 of SEQ ID NO:420, a CDR-H3 of SEQ ID NO:421, a CDR-L1 of SEQ ID NO:422, a CDR-L2 of SEQ ID NO:423, and a CDR-L1 of SEQ ID NO:424, as determined by the Exemplary numbering system.

In some embodiments, an antibody provided herein comprises a CDR-H1 of SEQ ID NO:457, a CDR-H2 of SEQ ID NO:458, a CDR-H3 of SEQ ID NO:459, a CDR-L1 of SEQ ID NO:460, a CDR-L2 of SEQ ID NO:461, and a CDR-L1 of SEQ ID NO:462, as determined by the Exemplary numbering system.

In some embodiments, an antibody provided herein comprises a CDR-H1 of SEQ ID NO:495, a CDR-H2 of SEQ ID NO:496, a CDR-H3 of SEQ ID NO:497, a CDR-L1 of SEQ ID NO:498, a CDR-L2 of SEQ ID NO:499, and a CDR-L1 of SEQ ID NO:500, as determined by the Exemplary numbering system.

In some embodiments, an antibody provided herein comprises a CDR-H1 of SEQ ID NO:533, a CDR-H2 of SEQ ID NO:534, a CDR-H3 of SEQ ID NO:535, a CDR-L1 of SEQ ID NO:536, a CDR-L2 of SEQ ID NO:537, and a CDR-L1 of SEQ ID NO:538, as determined by the Exemplary numbering system.

In some embodiments, an antibody provided herein comprises a CDR-H1 of SEQ ID NO:571, a CDR-H2 of SEQ ID NO:572, a CDR-H3 of SEQ ID NO:573, a CDR-L1 of SEQ ID NO:574, a CDR-L2 of SEQ ID NO:575, and a CDR-L1 of SEQ ID NO:576, as determined by the Exemplary numbering system.

In some embodiments, an antibody provided herein comprises a CDR-H1 of SEQ ID NO:609, a CDR-H2 of SEQ ID NO:610, a CDR-H3 of SEQ ID NO:611, a CDR-L1 of SEQ ID NO:612, a CDR-L2 of SEQ ID NO:613, and a CDR-L1 of SEQ ID NO:614, as determined by the Exemplary numbering system.

In some embodiments, an antibody provided herein comprises a CDR-H1 of SEQ ID NO:647, a CDR-H2 of SEQ ID NO:648, a CDR-H3 of SEQ ID NO:649, a CDR-L1 of SEQ ID NO:650, a CDR-L2 of SEQ ID NO:651, and a CDR-L1 of SEQ ID NO:652, as determined by the Exemplary numbering system.

In some embodiments, an antibody provided herein comprises a CDR-H1 of SEQ ID NO:685, a CDR-H2 of SEQ ID NO:686, a CDR-H3 of SEQ ID NO:687, a CDR-L1 of SEQ ID NO:688, a CDR-L2 of SEQ ID NO:689, and a CDR-L1 of SEQ ID NO:690, as determined by the Exemplary numbering system.

In some embodiments, an antibody provided herein comprises a CDR-H1 of SEQ ID NO:723, a CDR-H2 of SEQ ID NO:724, a CDR-H3 of SEQ ID NO:725, a CDR-L1 of SEQ ID NO:726, a CDR-L2 of SEQ ID NO:727, and a CDR-L1 of SEQ ID NO:728, as determined by the Exemplary numbering system.

### 2.2.5. Consensus Sequences

In some embodiments, provided herein is a first family of antibodies, wherein an antibody of such family comprises the following six CDR sequences: (a) a CDR-H1 having the sequence G-F-T-F-S-X₁-Y-A-M-X₂, wherein X₁ is D or S and X₂ is A or G (SEQ ID NO:773); (b) a CDR-H2 having the sequence X₃-I-S-G-S-G-G-L-T-Y-Y-A-D-S-V-K-G, wherein X₃ is A or T (SEQ ID NO:774); (c) a CDR-H3 having the sequence APYGYYMDV (SEQ ID NO:775); (d) a CDR-L1 having the sequence RASQSISSWLA (SEQ ID NO:776); (e) a CDR-L2 having the sequence KASSLES (SEQ ID NO:777); and (f) a CDR-L3 having the sequence QQYKSYIT (SEQ ID NO:778). In some embodiments, an antibody of such family comprises a V_{H} sequence of SEQ ID NO:761 and a V_{L} sequence of SEQ ID NO:762. In some embodiments, provided herein is an antibody within such first family.

In some embodiments, provided herein is a second family of antibodies, wherein an antibody of such family comprises the following six CDR sequences: (a) a CDR-H1 having the sequence G-Y-T-F-X₁-X₂-Y-G-I-S, wherein X₁ is D or R and X₂ is S or V (SEQ ID NO:779); (b) a CDR-H2 having the sequence W-X₃-A-P-Y-X₄-G-N-T-N-Y-A-Q-K-L-Q-G, wherein X₃ is I or V and X₄ is S or N (SEQ ID NO:780); (c) a CDR-H3 having the sequence D-A-G-T-Y-S-P-X₅-G-Y-G-M-D-V, wherein X₅ is F or Y (SEQ ID NO:781); (d) a CDR-L1 having the sequence X₆-A-S-X₇-S-I-X₈-X₉-W-L-A, wherein X₆ is R or Q, X₇ is Q, E, or H, Xₛ is S, D, or N, and X₉ is S or N (SEQ ID NO:782); (e) a CDR-L2 having the sequence X₁₀-A-X₁₁-X₁₂-L-E-X₁₃, wherein X₁₀ is K or S, X₁₁ is S or Y, X₁₂ is S, Y, or N, and X₁₃ is S or Y (SEQ ID NO:783); and (f) a CDR-L3 having the sequence Q-X₁₄-F-Q-X₁₅-L-P-P-F-T, wherein X₁₄ is Q, L, or R, and X₁₅ is S or K (SEQ ID NO:784). In some embodiments, an antibody of such family comprises a V_{H} sequence of SEQ ID NO:763 and a V_{L} sequence of SEQ ID NO:764. In some embodiments, provided herein is an antibody within such second family.

In some embodiments, provided herein is a third family of antibodies, wherein an antibody of such family comprises the following six CDR sequences: (a) a CDR-H1 having the sequence G-F-T-F-X₁-S-X₂-G-M-H, wherein X₁ is H or R and X₂ is R or Y (SEQ ID NO:785); (b) a CDR-H2 having the sequence VITYDGINKYYADSVEG (SEQ ID NO:786); (c) a CDR-H3 having the sequence DGVYYGVYDY (SEQ ID NO:787); (d) a CDR-L1 having the sequence KSSQSVLFSSNNKNYLA (SEQ ID NO:788); (e) a CDR-L2 having the sequence WASTRES (SEQ ID NO:789); and (f) a CDR-L3 having the sequence QQFHSYPLT (SEQ ID NO:790). In some embodiments, an antibody of such family comprises a V_{H} sequence of SEQ ID NO:765 and a V_{L} sequence of SEQ ID NO:766. In some embodiments, provided herein is an antibody within such third family.

In some embodiments, provided herein is a fourth family of antibodies, wherein an antibody of such family comprises the following six CDR sequences: (a) a CDR-H1 having the sequence GGTFSSNAIG (SEQ ID NO:791); (b) a CDR-H2 having the sequence SIIPIIGFANYAQKFQG (SEQ ID NO:792); (c) a CDR-H3 having the sequence DSGYYYGASSFGMDV (SEQ ID NO:793); (d) a CDR-L1 having the sequence RASQSVSSNLA (SEQ ID NO:794); (e) a CDR-L2 having the sequence GASTRAT (SEQ ID NO:795); and (f) a CDR-L3 having the sequence EQYNNLPLT (SEQ ID NO:796). In some embodiments, an antibody of such family comprises a V_{H} sequence of SEQ ID NO:767 and a V_{L} sequence of SEQ ID NO:768. In some embodiments, provided herein is an antibody within such fourth family.

In some embodiments, provided herein is a fifth family of antibodies, wherein an antibody of such family comprises the following six CDR sequences: (a) a CDR-H1 having the sequence G-G-S-X₁-S-S-G-X₂-Y-W-S, wherein X₁ is I or L and X₂ is Q or Y (SEQ ID NO:797); (b) a CDR-H2 having the sequence E-I-X₃-X₄-S-G-S-T-R-Y-N-P-S-L-K-S, wherein X₃ is Y or G and X₄ is Y or A (SEQ ID NO:798); (c) a CDR-H3 having the sequence D-X₅-P-Y-Y-Y-X₆-G-G-Y-Y-Y-Y-M-D-V, wherein X₅ is T or A and X₆ is E, G, or D (SEQ ID NO:799); (d) a CDR-L1 having the sequence R-A-S-X₇-S-V-X₈-S-S-X₉-L-A, wherein X₇ is Q, E, or D, Xₛ is S or D, and X₉ is Y or F (SEQ ID NO:800); (e) a CDR-L2 having the sequence G-A-X₁₀-X₁₁-R-X₁₂-X₁₃, wherein X₁₀ is S, D, F, or Y, X₁₁ is S or T, X₁₂ is A or Q, and X₁₃ is T or N (SEQ ID NO:801); and (f) a CDR-L3 having the sequence Q-Q-X₁₄-G-V-V-P-Y-T, wherein X₁₄ is V, A, or D (SEQ ID NO:802). In some embodiments, an antibody of such family comprises a V_{H} sequence of SEQ ID NO:769 and a V_{L} sequence of SEQ ID NO:770. In some embodiments, provided herein is an antibody within such fifth family.

In some embodiments, provided herein is a sixth family of antibodies, wherein an antibody of such family comprises the following six CDR sequences: (a) a CDR-H1 having the sequence GYTFANYYMH (SEQ ID NO:803); (b) a CDR-H2 having the sequence IINPSGGITVYAQKFQG (SEQ ID NO:804); (c) a CDR-H3 having the sequence GGSKVAALAFDI (SEQ ID NO:805); (d) a CDR-L1 having the sequence QASQDISNSLN (SEQ ID NO:806); (e) a CDR-L2 having the sequence DASNLET (SEQ ID NO:807); and (f) a CDR-L3 having the sequence QQYNFHPLT (SEQ ID NO:808). In some embodiments, an antibody of such family comprises a V_{H} sequence of SEQ ID NO:771 and a V_{L} sequence of SEQ ID NO:772. In some embodiments, provided herein is an antibody within such sixth family.

In some embodiments, provided herein is a seventh family of antibodies, wherein an antibody of such family comprises the following six CDR sequences: (a) a CDR-H1 having the sequence G-Y-T-F-D-X₁-Y-G-I-S, wherein X₁ is V or A (SEQ ID NO:872); (b) a CDR-H2 having the sequence W-I-A-P-Y-X₂-G-N-T-N-Y-A-Q-K-L-Q-G, wherein X₂ is N or S (SEQ ID NO:873); (c) a CDR-H3 having the sequence D-A-G-T-Y-S-P-F-G-Y-G-M-D-V (SEQ ID NO:874); (d) a CDR-L1 having the sequence X₃-A-S-X₄-S-I-X₅-X₆-W-L-A, wherein X₃ is R or Q, X₄ is Q or E, X₅ is S or N, and X₆ is S or N (SEQ ID NO:875); (e) a CDR-L2 having the sequence K-A-X₇-X₈-L-E-X₉, wherein X₇ is S or Y, Xₛ is S or N, and X₉ is S or Y (SEQ ID NO:876); and (f) a CDR-L3 having the sequence Q-X₁₀-F-Q-X₁₁-L-P-P-F-T, wherein X₁₀ is Q or L, and X₁₁ is S or K (SEQ ID NO:877). In some embodiments, an antibody of such family comprises a V_{H} sequence of SEQ ID NO:868 and a V_{L} sequence of SEQ ID NO:869. In some embodiments, provided herein is an antibody within such seventh family.

In some embodiments, provided herein is an eighth family of antibodies, wherein an antibody of such family comprises the following six CDR sequences: (a) a CDR-H1 having the sequence G-Y-T-F-R-S-Y-G-I-S(SEQ ID NO:878); (b) a CDR-H2 having the sequence W-V-A-P-Y-X₁-G-N-T-N-Y-A-Q-K-L-Q-G, wherein X₁ is S or N (SEQ ID NO:879); (c) a CDR-H3 having the sequence D-A-G-T-Y-S-P-Y-G-Y-G-M-D-V (SEQ ID NO:880); (d) a CDR-L1 having the sequence X₂-A-S-X₃-S-I-X₄-S-W-L-A, wherein X₂ is R or Q, X₃ is Q or H, X₄ is S or D (SEQ ID NO:881); (e) a CDR-L2 having the sequence X₅-A-S-X₆-L-E-S, wherein X₅ is K or S, X₆ is S or Y (SEQ ID NO:882); and (f) a CDR-L3 having the sequence Q-X₇-F-Q-S-L-P-P-F-T, wherein X₇ is Q, L, or R (SEQ ID NO:883). In some embodiments, an antibody of such family comprises a V_{H} sequence of SEQ ID NO:870 and a VL sequence of SEQ ID NO:871. In some embodiments, provided herein is an antibody within such eighth family.

### 2.2.6. Functional Properties of Antibody Variants

As described above, and elsewhere in this disclosure, provided herein are antibody variants defined based on percent identity to an illustrative antibody sequence provided herein, or substitution of amino acid residues in comparison to an illustrative antibody sequence provided herein.

In some embodiments, a variant of an antibody provided herein has specificity for hTF. In some embodiments, a variant of an antibody provided herein has specificity for cTF. In some embodiments, a variant of an antibody provided herein has specificity for mTF. In some embodiments, a variant of an antibody provided herein has specificity for hTF and cTF. In some embodiments, a variant of an antibody provided herein has specificity for hTF and mTF. In some embodiments, a variant of an antibody provided herein has specificity for cTF and mTF. In some embodiments, a variant of an antibody provided herein has specificity for hTF, cTF and mTF.

In some embodiments, a variant of an antibody that is derived from an illustrative antibody sequence provided herein retains affinity, as measured by K_{D}, for hTF that is within about 1.5-fold, about 2-fold, about 3-fold, about 4-fold, about 5-fold, about 6-fold, about 7-fold, about 8-fold, about 9-fold or about 10-fold the affinity of such illustrative antibody. In some embodiments, a variant of an antibody that is derived from an illustrative antibody sequence provided herein retains affinity, as measured by K_{D}, for cTF that is within about 1.5-fold, about 2-fold, about 3-fold, about 4-fold, about 5-fold, about 6-fold, about 7-fold, about 8-fold, about 9-fold or about 10-fold the affinity of such illustrative antibody. In some embodiments, a variant of an antibody that is derived from an illustrative antibody sequence provided herein retains affinity, as measured by K_{D}, for mTF that is within about 1.5-fold, about 2-fold, about 3-fold, about 4-fold, about 5-fold, about 6-fold, about 7-fold, about 8-fold, about 9-fold or about 10-fold the affinity of such illustrative antibody. In some embodiments, a variant of an antibody that is derived from an illustrative antibody sequence provided herein retains affinity, as measured by K_{D}, for both hTF and cTF that is within about 1.5-fold, about 2-fold, about 3-fold, about 4-fold, about 5-fold, about 6-fold, about 7-fold, about 8-fold, about 9-fold or about 10-fold the affinity of such illustrative antibody. In some embodiments, a variant of an antibody that is derived from an illustrative antibody sequence provided herein retains affinity, as measured by K_{D}, for both hTF and mTF that is within about 1.5-fold, about 2-fold, about 3-fold, about 4-fold, about 5-fold, about 6-fold, about 7-fold, about 8-fold, about 9-fold or about 10-fold the affinity of such illustrative antibody. In some embodiments, a variant of an antibody that is derived from an illustrative antibody sequence provided herein retains affinity, as measured by K_{D}, for both cTF and mTF that is within about 1.5-fold, about 2-fold, about 3-fold, about 4-fold, about 5-fold, about 6-fold, about 7-fold, about 8-fold, about 9-fold or about 10-fold the affinity of such illustrative antibody. In some embodiments, a variant of an antibody that is derived from an illustrative antibody sequence provided herein retains affinity, as measured by K_{D}, for all three of hTF, cTF and mTF that is within about 1.5-fold, about 2-fold, about 3-fold, about 4-fold, about 5-fold, about 6-fold, about 7-fold, about 8-fold, about 9-fold or about 10-fold the affinity of such illustrative antibody.

In some embodiments, a variant of an antibody provided herein retains the ability to inhibit TF signaling, as measured by one or more assays or biological effects described herein. In some embodiments, a variant of an antibody provided herein retains the normal function of TF in the blood coagulation processes.

In some embodiments, a variant of an antibody provided herein competes for binding to TF with an antibody selected from 1F, 1G, 25A, 25A3, 25A5, 25A5-T, 25G, 25G1, 25G9, 29D, 29E, 39A, 43B, 43B1, 43B7, 43D, 43D7, 43D8, 43E, 43Ea, and 54E, each as provided in Table 13 of this disclosure. In some embodiments, a variant of an antibody provided herein competes for binding to TF with an antibody selected from 25A, 25A3, 25A5, 25A5-T, 25G, 25G1, and 25G9. In some embodiments, a variant of an antibody provided herein competes for binding to TF with an antibody selected from 43B, 43B1, 43B7, 43D, 43D7, 43D8, 43E, and 43Ea. In some embodiments, a variant of an antibody provided herein competes for binding to TF with an antibody selected from 25A, 25A3, 25A5, 25A5-T, 25G, 25G1, 25G9, 43B, 43B1, 43B7, 43D, 43D7, 43D8, 43E, and 43Ea. In some embodiments, a variant of an antibody provided herein competes for binding to TF with an antibody selected from 1F, 1G, 29D, 29E, 39A, or 54E.

In some embodiments, a variant of an antibody provided herein allows human thrombin generation as determined by thrombin generation assay (TGA). In some embodiments, a variant of an antibody provided herein does not inhibit human thrombin generation as determined by thrombin generation assay (TGA).

In some embodiments, a variant of an antibody provided herein binds human TF at a human TF binding site that is distinct from a human TF binding site bound by human FX. In some embodiments, a variant of an antibody provided herein does not interfere with the ability of TF:FVIIa to convert FX into FXa.

In some embodiments, a variant of an antibody provided herein binds human TF at a human TF binding site that is distinct from a human TF binding site bound by human FVIIa. In some embodiments, a variant of an antibody provided herein does not compete for binding to human TF with human FVIIa.

In some embodiments, a variant of an antibody provided herein inhibits FVIIa-dependent TF signaling.

In some embodiments, a variant of an antibody provided herein binds mouse TF (SEQ ID NO:817). In some embodiments, a variant of an antibody provided herein binds mouse TF with an affinity lower (as indicated by higher K_{D}) than the affinity of the antibody for hTF. In some embodiments, a variant of an antibody provided herein does not bind mTF.

In some embodiments, a variant of an antibody provided herein binds pig TF (SEQ ID NO:824). In some embodiments, a variant of an antibody provided herein binds pig TF with an affinity lower (as indicated by higher K_{D}) than the affinity of the antibody for hTF. In some embodiments, a variant of an antibody provided herein does not bind pTF.

In some embodiments, a variant of an antibody provided herein binds the same epitope of TF as such antibody.

### 2.2.7. Other Functional Properties of Antibodies

In some embodiments, an antibody provided herein has one or more of the characteristics listed in the following (a)-(dd): (a) binds human TF at a human TF binding site that is distinct from a human TF binding site bound by human FVIIa; (b) does not inhibit human thrombin generation as determined by thrombin generation assay (TGA); (c) does not reduce the thrombin peak on a thrombin generation curve (Peak IIa) compared to an isotype control; (d) does not increase the time from the assay start to the thrombin peak on a thrombin generation curve (ttPeak) compared to an isotype control; (e) does not decrease the endogenous thrombin potential (ETP) as determined by the area under a thrombin generation curve compared to an isotype control; (f) allows human thrombin generation as determined by thrombin generation assay (TGA); (g) maintains the thrombin peak on a thrombin generation curve (Peak IIa) compared to an isotype control; (h) maintains the time from the assay start to the thrombin peak on a thrombin generation curve (ttPeak) compared to an isotype control; (i) preserves the endogenous thrombin potential (ETP) as determined by the area under a thrombin generation curve compared to an isotype control; (j) binds human TF at a human TF binding site that is distinct from a human TF binding site bound by human FX; (k) does not interfere with the ability of TF:FVIIa to convert FX into FXa; (1) does not compete for binding to human TF with human FVIIa; (m) inhibits FVIIa-dependent TF signaling; (n) binds to cynomolgus TF; (o) binds to mouse TF; (p) binds to rabbit TF; (q) binds to pig TF; (r) reduces lesion size in a swine choroidal neovascularization (CNV) model; (s) the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (t) the binding between the antibody and a variant TF extracellular domain comprising a mutation at amino acid residue 68 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay; (u) the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (v) the binding between the antibody and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (w) the binding between the antibody and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (x) the binding between the antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (y) the binding between the antibody and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (z) the binding between the antibody and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (aa) the binding between the antibody and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (bb) the binding between the antibody and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (cc) the binding between the antibody and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; and (dd) the binding between the antibody and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay. In some embodiments, an antibody provided herein has two or more of the characteristics listed in the foregoing (a)-(dd). In some embodiments, an antibody provided herein has three or more of the characteristics listed in the foregoing (a)-(dd). In some embodiments, an antibody provided herein has four or more of the characteristics listed in the foregoing (a)-(dd). In some embodiments, an antibody provided herein has five or more of the characteristics listed in the foregoing (a)-(dd). In some embodiments, an antibody provided herein has six or more of the characteristics listed in the foregoing (a)-(dd). In some embodiments, an antibody provided herein has seven or more of the characteristics listed in the foregoing (a)-(dd). In some embodiments, an antibody provided herein has eight or more of the characteristics listed in the foregoing (a)-(dd). In some embodiments, an antibody provided herein has nine or more of the characteristics listed in the foregoing (a)-(dd). In some embodiments, an antibody provided herein has ten or more of the characteristics listed in the foregoing (a)-(dd). In some embodiments, an antibody provided herein has eleven or more of the characteristics listed in the foregoing (a)-(dd). In some embodiments, an antibody provided herein has twelve or more of the characteristics listed in the foregoing (a)-(dd). In some embodiments, an antibody provided herein has thirteen or more of the characteristics listed in the foregoing (a)-(dd). In some embodiments, an antibody provided herein has fourteen or more of the characteristics listed in the foregoing (a)-(dd). In some embodiments, an antibody provided herein has fifteen or more of the characteristics listed in the foregoing (a)-(dd). In some embodiments, an antibody provided herein has sixteen or more of the characteristics listed in the foregoing (a)-(dd). In some embodiments, an antibody provided herein has seventeen or more of the characteristics listed in the foregoing (a)-(dd). In some embodiments, an antibody provided herein has eighteen or more of the characteristics listed in the foregoing (a)-(dd). In some embodiments, an antibody provided herein has nineteen or more of the characteristics listed in the foregoing (a)-(dd). In some embodiments, an antibody provided herein has twenty or more of the characteristics listed in the foregoing (a)-(dd). In some embodiments, an antibody provided herein has twenty-one or more of the characteristics listed in the foregoing (a)-(dd). In some embodiments, an antibody provided herein has twenty-two or more of the characteristics listed in the foregoing (a)-(dd). In some embodiments, an antibody provided herein has twenty-three of the characteristics listed in the foregoing (a)-(dd). In some embodiments, an antibody provided herein has twenty-four of the characteristics listed in the foregoing (a)-(dd). In some embodiments, an antibody provided herein has twenty-five of the characteristics listed in the foregoing (a)-(dd). In some embodiments, an antibody provided herein has twenty-six of the characteristics listed in the foregoing (a)-(dd). In some embodiments, an antibody provided herein has twenty-seven of the characteristics listed in the foregoing (a)-(dd). In some embodiments, an antibody provided herein has twenty-eight of the characteristics listed in the foregoing (a)-(dd). In some embodiments, an antibody provided herein has twenty-nine of the characteristics listed in the foregoing (a)-(dd). In some embodiments, an antibody provided herein has all thirty of the characteristics listed in the foregoing (a)-(dd).

In some embodiments, an antibody provided herein has one or more of the characteristics listed in the following (a)-(dd): (a) binds human TF at a human TF binding site that is distinct from a human TF binding site bound by human FVIIa; (b) does not inhibit human thrombin generation as determined by thrombin generation assay (TGA); (c) does not reduce the thrombin peak on a thrombin generation curve (Peak IIa) compared to an isotype control; (d) does not increase the time from the assay start to the thrombin peak on a thrombin generation curve (ttPeak) compared to an isotype control; (e) does not decrease the endogenous thrombin potential (ETP) as determined by the area under a thrombin generation curve compared to an isotype control; (f) allows human thrombin generation as determined by thrombin generation assay (TGA); (g) maintains the thrombin peak on a thrombin generation curve (Peak IIa) compared to an isotype control; (h) maintains the time from the assay start to the thrombin peak on a thrombin generation curve (ttPeak) compared to an isotype control; (i) preserves the endogenous thrombin potential (ETP) as determined by the area under a thrombin generation curve compared to an isotype control; (j) binds human TF at a human TF binding site that is distinct from a human TF binding site bound by human FX; (k) does not interfere with the ability of TF:FVIIa to convert FX into FXa; (1) does not compete for binding to human TF with human FVIIa; (m) inhibits FVIIa-dependent TF signaling; (n) binds to cynomolgus TF; (o) binds to mouse TF; (p) binds to rabbit TF; (q) binds to pig TF; (r) reduces lesion size in a swine choroidal neovascularization (CNV) model; (s) the binding between the antibody and a variant TF extracellular domain comprising a mutation K149N of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (t) the binding between the antibody and a variant TF extracellular domain comprising a mutation K68N of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay; (u) the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (v) the binding between the antibody and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (w) the binding between the antibody and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (x) the binding between the antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (y) the binding between the antibody and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (z) the binding between the antibody and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (aa) the binding between the antibody and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (bb) the binding between the antibody and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (cc) the binding between the antibody and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; and (dd) the binding between the antibody and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay. In some embodiments, an antibody provided herein has two or more of the characteristics listed in the foregoing (a)-(dd). In some embodiments, an antibody provided herein has three or more of the characteristics listed in the foregoing (a)-(dd). In some embodiments, an antibody provided herein has four or more of the characteristics listed in the foregoing (a)-(dd). In some embodiments, an antibody provided herein has five or more of the characteristics listed in the foregoing (a)-(dd). In some embodiments, an antibody provided herein has six or more of the characteristics listed in the foregoing (a)-(dd). In some embodiments, an antibody provided herein has seven or more of the characteristics listed in the foregoing (a)-(dd). In some embodiments, an antibody provided herein has eight or more of the characteristics listed in the foregoing (a)-(dd). In some embodiments, an antibody provided herein has nine or more of the characteristics listed in the foregoing (a)-(dd). In some embodiments, an antibody provided herein has ten or more of the characteristics listed in the foregoing (a)-(dd). In some embodiments, an antibody provided herein has eleven or more of the characteristics listed in the foregoing (a)-(dd). In some embodiments, an antibody provided herein has twelve or more of the characteristics listed in the foregoing (a)-(dd). In some embodiments, an antibody provided herein has thirteen or more of the characteristics listed in the foregoing (a)-(dd). In some embodiments, an antibody provided herein has fourteen or more of the characteristics listed in the foregoing (a)-(dd). In some embodiments, an antibody provided herein has fifteen or more of the characteristics listed in the foregoing (a)-(dd). In some embodiments, an antibody provided herein has sixteen or more of the characteristics listed in the foregoing (a)-(dd). In some embodiments, an antibody provided herein has seventeen or more of the characteristics listed in the foregoing (a)-(dd). In some embodiments, an antibody provided herein has eighteen or more of the characteristics listed in the foregoing (a)-(dd). In some embodiments, an antibody provided herein has nineteen or more of the characteristics listed in the foregoing (a)-(dd). In some embodiments, an antibody provided herein has twenty or more of the characteristics listed in the foregoing (a)-(dd). In some embodiments, an antibody provided herein has twenty-one or more of the characteristics listed in the foregoing (a)-(dd). In some embodiments, an antibody provided herein has twenty-two or more of the characteristics listed in the foregoing (a)-(dd). In some embodiments, an antibody provided herein has twenty-three of the characteristics listed in the foregoing (a)-(dd). In some embodiments, an antibody provided herein has twenty-four of the characteristics listed in the foregoing (a)-(dd). In some embodiments, an antibody provided herein has twenty-five of the characteristics listed in the foregoing (a)-(dd). In some embodiments, an antibody provided herein has twenty-six of the characteristics listed in the foregoing (a)-(dd). In some embodiments, an antibody provided herein has twenty-seven of the characteristics listed in the foregoing (a)-(dd). In some embodiments, an antibody provided herein has twenty-eight of the characteristics listed in the foregoing (a)-(dd). In some embodiments, an antibody provided herein has twenty-nine of the characteristics listed in the foregoing (a)-(dd). In some embodiments, an antibody provided herein has all thirty of the characteristics listed in the foregoing (a)-(dd).

In some embodiments, an antibody provided herein exhibits a combination of characteristics comprising two or more of characteristics listed in the following (a)-(dd): (a) binds human TF at a human TF binding site that is distinct from a human TF binding site bound by human FVIIa; (b) does not inhibit human thrombin generation as determined by thrombin generation assay (TGA); (c) does not reduce the thrombin peak on a thrombin generation curve (Peak IIa) compared to an isotype control; (d) does not increase the time from the assay start to the thrombin peak on a thrombin generation curve (ttPeak) compared to an isotype control; (e) does not decrease the endogenous thrombin potential (ETP) as determined by the area under a thrombin generation curve compared to an isotype control; (f) allows human thrombin generation as determined by thrombin generation assay (TGA); (g) maintains the thrombin peak on a thrombin generation curve (Peak IIa) compared to an isotype control; (h) maintains the time from the assay start to the thrombin peak on a thrombin generation curve (ttPeak) compared to an isotype control; (i) preserves the endogenous thrombin potential (ETP) as determined by the area under a thrombin generation curve compared to an isotype control; (j) binds human TF at a human TF binding site that is distinct from a human TF binding site bound by human FX; (k) does not interfere with the ability of TF:FVIIa to convert FX into FXa; (l) does not compete for binding to human TF with human FVIIa; (m) inhibits FVIIa-dependent TF signaling; (n) binds to cynomolgus TF; (o) binds to mouse TF; (p) binds to rabbit TF; (q) binds to pig TF; (r) reduces lesion size in a swine choroidal neovascularization (CNV) model; (s) the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (t) the binding between the antibody and a variant TF extracellular domain comprising a mutation at amino acid residue 68 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay; (u) the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (v) the binding between the antibody and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (w) the binding between the antibody and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (x) the binding between the antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (y) the binding between the antibody and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (z) the binding between the antibody and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (aa) the binding between the antibody and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (bb) the binding between the antibody and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (cc) the binding between the antibody and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; and (dd) the binding between the antibody and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of characteristics comprising two or more of characteristics listed in the following (a)-(dd): (a) binds human TF at a human TF binding site that is distinct from a human TF binding site bound by human FVIIa; (b) does not inhibit human thrombin generation as determined by thrombin generation assay (TGA); (c) does not reduce the thrombin peak on a thrombin generation curve (Peak IIa) compared to an isotype control; (d) does not increase the time from the assay start to the thrombin peak on a thrombin generation curve (ttPeak) compared to an isotype control; (e) does not decrease the endogenous thrombin potential (ETP) as determined by the area under a thrombin generation curve compared to an isotype control; (f) allows human thrombin generation as determined by thrombin generation assay (TGA); (g) maintains the thrombin peak on a thrombin generation curve (Peak IIa) compared to an isotype control; (h) maintains the time from the assay start to the thrombin peak on a thrombin generation curve (ttPeak) compared to an isotype control; (i) preserves the endogenous thrombin potential (ETP) as determined by the area under a thrombin generation curve compared to an isotype control; (j) binds human TF at a human TF binding site that is distinct from a human TF binding site bound by human FX; (k) does not interfere with the ability of TF:FVIIa to convert FX into FXa; (l) does not compete for binding to human TF with human FVIIa; (m) inhibits FVIIa-dependent TF signaling; (n) binds to cynomolgus TF; (o) binds to mouse TF; (p) binds to rabbit TF; (q) binds to pig TF; (r) reduces lesion size in a swine choroidal neovascularization (CNV) model; (s) the binding between the antibody and a variant TF extracellular domain comprising a mutation K149N of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (t) the binding between the antibody and a variant TF extracellular domain comprising a mutation K68N of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay; (u) the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; (v) the binding between the antibody and a human TF extracellular domain with amino acid residues 1-77 of the sequence shown in SEQ ID NO:810 replaced by rat TF extracellular domain amino acid residues 1-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (w) the binding between the antibody and a human TF extracellular domain with amino acid residues 39-77 of the sequence shown in SEQ ID NO:810 replaced by rat TF extracellular domain amino acid residues 38-76 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (x) the binding between the antibody and a human TF extracellular domain with amino acid residues 94-107 of the sequence shown in SEQ ID NO:810 replaced by rat TF extracellular domain amino acid residues 99-112 of the sequence shown in SEQ ID NO:838 is greater than 50% of the binding between the antibody and the extracellular domain of TF of the sequence shown in SEQ ID NO:810, as determined by the median fluorescence intensity value of the antibody relative to an isotype control in a live cell staining assay; (y) the binding between the antibody and a human TF extracellular domain with amino acid residues 146-158 of the sequence shown in SEQ ID NO:810 replaced by rat TF extracellular domain amino acid residues 151-163 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (z) the binding between the antibody and a human TF extracellular domain with amino acid residues 159-219 of the sequence shown in SEQ ID NO:810 replaced by rat TF extracellular domain amino acid residues 164-224 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (aa) the binding between the antibody and a human TF extracellular domain with amino acid residues 159-189 of the sequence shown in SEQ ID NO:810 replaced by rat TF extracellular domain amino acid residues 164-194 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (bb) the binding between the antibody and a human TF extracellular domain with amino acid residues 159-174 of the sequence shown in SEQ ID NO:810 replaced by rat TF extracellular domain amino acid residues 164-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; (cc) the binding between the antibody and a human TF extracellular domain with amino acid residues 167-174 of the sequence shown in SEQ ID NO:810 replaced by rat TF extracellular domain amino acid residues 172-179 of the sequence shown in SEQ ID NO:838 is less than 50% of the binding between the antibody and the extracellular domain of TF of the sequence shown in SEQ ID NO:810, as determined by the median fluorescence intensity value of the antibody relative to an isotype control in a live cell staining assay; and (dd) the binding between the antibody and a rat TF extracellular domain with amino acid residues 141-194 of the sequence shown in SEQ ID NO:838 replaced by human TF extracellular domain amino acid residues 136-189 of the sequence shown in SEQ ID NO:810 is greater than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; does not inhibit human thrombin generation as determined by thrombin generation assay (TGA); and the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; does not inhibit human thrombin generation as determined by thrombin generation assay (TGA); and the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; allows human thrombin generation as determined by thrombin generation assay (TGA); and the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; allows human thrombin generation as determined by thrombin generation assay (TGA); and the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; does not inhibit human thrombin generation as determined by thrombin generation assay (TGA); the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; does not inhibit human thrombin generation as determined by thrombin generation assay (TGA); the binding between the antibody and a variant TF extracellular domain comprising a mutation K149N of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; allows human thrombin generation as determined by thrombin generation assay (TGA); the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; allows human thrombin generation as determined by thrombin generation assay (TGA); the binding between the antibody and a variant TF extracellular domain comprising a mutation K149N of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; does not inhibit human thrombin generation as determined by thrombin generation assay (TGA); binds to cynomolgus TF; the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; does not inhibit human thrombin generation as determined by thrombin generation assay (TGA); binds to cynomolgus TF; the binding between the antibody and a variant TF extracellular domain comprising a mutation K149N of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; allows human thrombin generation as determined by thrombin generation assay (TGA); binds to cynomolgus TF; the binding between the antibody and a variant TF extracellular domain comprising a mutation at amino acid residue 149 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations at amino acid residues 171 and 197 of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

In some embodiments, an antibody provided herein exhibits a combination of the characteristics listed in the following: binds human TF at a human TF binding site that is distinct from a human TF binding site bound by human FVIIa; allows human thrombin generation as determined by thrombin generation assay (TGA); binds to cynomolgus TF; the binding between the antibody and a variant TF extracellular domain comprising a mutation K149N of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay; and the binding between the antibody and a variant TF extracellular domain comprising mutations N171H and T197K of the sequence shown in SEQ ID NO:810 is less than 50% of the binding between the antibody and the extracellular domain of TF of the sequence shown in SEQ ID NO:810, as determined by the median fluorescence intensity value of the antibody relative to an isotype control in a live cell staining assay.

### 2.3. Affinity and Other Properties of TF Antibodies

### 2.3.1. Affinity of TF Antibodies

In some embodiments, the affinity of an antibody provided herein for TF as indicated by K_{D}, is less than about 10⁻⁵ M, less than about 10⁻⁶ M, less than about 10⁻⁷ M, less than about 10⁻⁸ M, less than about 10⁻⁹ M, less than about 10⁻¹⁰ M, less than about 10⁻¹¹ M, or less than about 10⁻¹² M. In some embodiments, the affinity of the antibody is between about 10⁻⁷ M and 10⁻¹² M. In some embodiments, the affinity of the antibody is between about 10⁻⁷ M and 10⁻¹¹ M. In some embodiments, the affinity of the antibody is between about 10⁻⁷ M and 10⁻¹⁰ M. In some embodiments, the affinity of the antibody is between about 10⁻⁷ M and 10⁻⁹ M. In some embodiments, the affinity of the antibody is between about 10⁻⁷ M and 10⁻⁸ M. In some embodiments, the affinity of the antibody is between about 10⁻⁸ M and 10⁻¹² M. In some embodiments, the affinity of the antibody is between about 10⁻⁸ M and 10⁻¹¹ M. In some embodiments, the affinity of the antibody is between about 10⁻⁹ M and 10⁻¹¹ M. In some embodiments, the affinity of the antibody is between about 10⁻¹⁰ M and 10⁻¹¹ M.

In some embodiments, the K_{D} value of an antibody provided herein for cTF is no more than 15× of the K_{D} value of the antibody for hTF. In some embodiments, the K_{D} value of an antibody provided herein for cTF is no more than 10× of the K_{D} value of the antibody for hTF. In some embodiments, the K_{D} value of an antibody provided herein for cTF is no more than 8× of the K_{D} value of the antibody for hTF. In some embodiments, the K_{D} value of an antibody provided herein for cTF is no more than 5× of the K_{D} value of the antibody for hTF. In some embodiments, the K_{D} value of an antibody provided herein for cTF is no more than 3× of the K_{D} value of the antibody for hTF. In some embodiments, the K_{D} value of an antibody provided herein for cTF is no more than 2× of the K_{D} value of the antibody for hTF.

In some embodiments, the K_{D} value of an antibody provided herein for mTF is no more than 20× of the K_{D} value of the antibody for hTF. In some embodiments, the K_{D} value of an antibody provided herein for mTF is no more than 15× of the K_{D} value of the antibody for hTF. In some embodiments, the K_{D} value of an antibody provided herein for mTF is no more than 10× of the K_{D} value of the antibody for hTF. In some embodiments, the K_{D} value of an antibody provided herein for mTF is no more than 5× of the K_{D} value of the antibody for hTF. In some embodiments, the K_{D} value of an antibody provided herein for mTF is no more than 2× of the K_{D} value of the antibody for hTF.

In some embodiments, the affinity of an antibody provided herein for hTF as indicated by K_{D} measured by Biacore, as set forth in Table 5 is selected from about 0.31 nM, about 6.20 nM, about 0.36 nM, about 0.08 nM, about 23.0 nM, about 0.94 nM, about 13.3 nM, about 0.47 nM, about 0.09 nM, about 1.75 nM, about 0.07 nM, about 0.14 nM, about 2.09 nM, about 0.06 nM, about 0.15 nM, about 1.46 nM, about 1.60 nM, and about 0.42 nM.

In some embodiments, such affinity as indicated by K_{D} ranges from about 23.0 nM to about 0.06 nM. In some embodiments, such is about 23.0 nM or less.

In some embodiments, the affinity of an antibody provided herein for hTF as indicated by K_{D} measured by ForteBio, as set forth in Table 5 is selected from about 1.28 nM, about 2.20 nM, about 8.45 nM, about 1.67 nM, about 0.64 nM, about 21.9 nM, about 3.97 nM, about 35.8 nM, about 3.30 nM, about 2.32 nM, about 0.83 nM, about 2.40 nM, about 0.96 nM, about 0.86 nM, about 3.84 nM, about 1.02 nM, about 1.61 nM, about 2.52 nM, about 2.28 nM, and about 1.59 nM. In some embodiments, such affinity as indicated by K_{D} ranges from about 35.8 nM to about 0.64 nM. In some embodiments, such K_{D} is about 35.8 nM or less.

In some embodiments, the affinity of an antibody provided herein for cTF as indicated by K_{D} measured by Biacore, as set forth in Table 5 is selected from about 0.26 nM, about 5.42 nM, about 0.21 nM, about 0.04 nM, about 18.0 nM, about 0.78 nM, about 16.4 nM, about 5.06 nM, about 0.08 nM, about 5.64 nM, about 0.12 nM, about 0.24 nM, about 5.66 nM, about 0.39 nM, about 5.69 nM, about 6.42 nM, and about 1.83 nM. In some embodiments, such affinity as indicated by K_{D} ranges from about 18.0 nM to about 0.04 nM. In some embodiments, such K_{D} is about 18.0 nM or less.

In some embodiments, the affinity of an antibody provided herein for cTF as indicated by K_{D} measured by ForteBio, as set forth in Table 5 is selected from about 1.43 nM, about 2.70 nM, about 7.65 nM, about 1.36 nM, about 0.76 nM, about 17.5 nM, about 4.99 nM, about 42.9 nM, about 12.0 nM, about 15.0 nM, about 0.57 nM, about 3.40 nM, about 1.05 nM, about 0.94 nM, about 4.12 nM, about 1.11 nM, about 1.96 nM, about 4.07 nM, about 2.71 nM, and about 4.16 nM. In some embodiments, such affinity as indicated by K_{D} ranges from about 42.9 nM to about 0.57 nM. In some embodiments, such K_{D} is about 42.9 nM or less.

In some embodiments, the affinity of an antibody provided herein for mTF as indicated by K_{D} measured by Biacore, as set forth in Table 5 is selected from about 5.4 nM, about 2.9 nM, about 21 nM, and about 2.4 nM. In some embodiments, such affinity as indicated by K_{D} ranges from about 21 nM to about 2.4 nM. In some embodiments, such K_{D} is about 21 nM or less.

In some embodiments, the affinity of an antibody provided herein for mTF as indicated by K_{D} measured by ForteBio, as set forth in Table 5 is selected from about 263 nM, about 131 nM, about 188 nM, about 114 nM, about 34.2 nM, about 9.16 nM, about 161 nM, about 72.1 nM, about 360 nM, about 281 nM, about 41.4 nM, about 6.12 nM, about 121 nM, and about 140 nM. In some embodiments, such affinity as indicated by K_{D} ranges from about 360 nM to about 6.12 nM. In some embodiments, such K_{D} is about 360 nM or less.

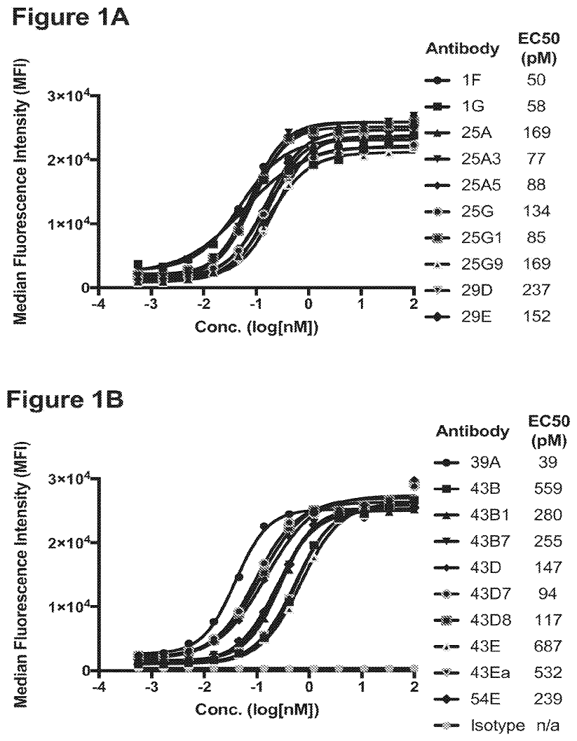

In some embodiments, the affinity of an antibody provided herein for hTF as indicated by EC₅₀ measured with human TF-positive HCT-116 cells, as set forth in FIGS. 1A and 1B is selected from about 50 pM, about 58 pM, about 169 pM, about 77 pM, about 88 pM, about 134 pM, about 85 pM, about 237 pM, about 152 pM, about 39 pM, about 559 pM, about 280 pM, about 255 pM, about 147 pM, about 94 pM, about 117 pM, about 687 pM, about 532 pM, and about 239 pM. In some embodiments, such affinity ranges from about 687 pM to about 39 pM. In some embodiments, such EC₅₀ is about 687 pM or less.

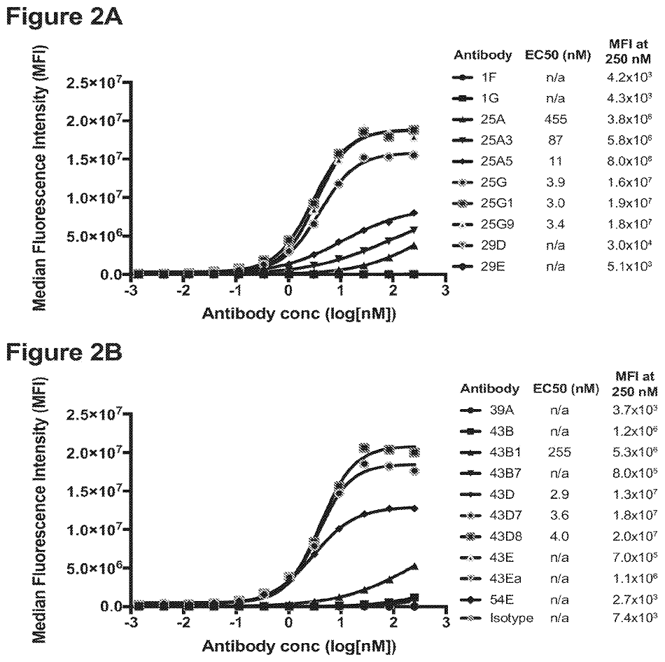

In some embodiments, the affinity of an antibody provided herein for mTF as indicated by EC₅₀ measured with mouse TF-positive CHO cells, as set forth in FIGS. 2A and 2B is selected from about 455 nM, about 87 nM, about 11 nM, about 3.9 nM, about 3.0 nM, about 3.4 nM, about 255 nM, about 2.9 nM, about 3.6 nM, and about 4.0 nM. In some embodiments, such affinity ranges from about 455 nM to about 2.9 nM. In some embodiments, such EC₅₀ is about 455 pM or less.

In some embodiments, the K_{D} value of an antibody provided herein for pTF is no more than 20× of the K_{D} value of the antibody for hTF. In some embodiments, the K_{D} value of an antibody provided herein for pTF is no more than 15× of the K_{D} value of the antibody for hTF. In some embodiments, the K_{D} value of an antibody provided herein for pTF is no more than 10× of the K_{D} value of the antibody for hTF. In some embodiments, the K_{D} value of an antibody provided herein for pTF is no more than 5× of the K_{D} value of the antibody for hTF. In some embodiments, the K_{D} value of an antibody provided herein for pTF is no more than 2× of the K_{D} value of the antibody for hTF.

In some embodiments, the affinity of an antibody provided herein for pTF as indicated by K_{D} measured by Biacore, as set forth in Table 40 is 3.31 nM or 12.9 nM.

### 2.3.2. Thrombin Generation in the Presence of TF Antibodies

In some embodiments, the TF antibodies provided herein do not inhibit human thrombin generation as determined by thrombin generation assay (TGA). In certain embodiments, the TF antibodies provided herein allow human thrombin generation as determined by thrombin generation assay (TGA).

In some embodiments, the percent peak thrombin generation (% Peak IIa) is at least 40% in the presence of no less than 100 nM TF antibody compared to the control conditions without the antibody, as determined by thrombin generation assay (TGA). In some embodiments, the % Peak IIa is at least 50% in the presence of no less than 100 nM TF antibody compared to the control conditions without the antibody, as determined by thrombin generation assay (TGA). In some embodiments, the % Peak IIa is at least 60% in the presence of no less than 100 nM TF antibody compared to the control conditions without the antibody, as determined by thrombin generation assay (TGA). In some embodiments, the % Peak IIa is at least 70% in the presence of no less than 100 nM TF antibody compared to the control conditions without the antibody, as determined by thrombin generation assay (TGA). In some embodiments, the % Peak IIa is at least 80% in the presence of no less than 100 nM TF antibody compared to the control conditions without the antibody, as determined by thrombin generation assay (TGA). In some embodiments, the % Peak IIa is at least 90% in the presence of no less than 100 nM TF antibody compared to the control conditions without the antibody, as determined by thrombin generation assay (TGA). In some embodiments, the % Peak IIa is at least 95% in the presence of no less than 100 nM TF antibody compared to the control conditions without the antibody, as determined by thrombin generation assay (TGA). In some embodiments, the % Peak IIa is at least 99% in the presence of no less than 100 nM TF antibody compared to the control conditions without the antibody, as determined by thrombin generation assay (TGA).

In some embodiments, the % Peak IIa is at least 40% in the presence of no less than 50 nM TF antibody compared to the control conditions without the antibody, as determined by thrombin generation assay (TGA). In some embodiments, the % Peak IIa is at least 50% in the presence of no less than 50 nM TF antibody compared to the control conditions without the antibody, as determined by thrombin generation assay (TGA). In some embodiments, the % Peak IIa is at least 60% in the presence of no less than 50 nM TF antibody compared to the control conditions without the antibody, as determined by thrombin generation assay (TGA). In some embodiments, the % Peak IIa is at least 70% in the presence of no less than 50 nM TF antibody compared to the control conditions without the antibody, as determined by thrombin generation assay (TGA). In some embodiments, the % Peak IIa is at least 80% in the presence of no less than 50 nM TF antibody compared to the control conditions without the antibody, as determined by thrombin generation assay (TGA). In some embodiments, the % Peak IIa is at least 90% in the presence of no less than 50 nM TF antibody compared to the control conditions without the antibody, as determined by thrombin generation assay (TGA). In some embodiments, the % Peak IIa is at least 95% in the presence of no less than 50 nM TF antibody compared to the control conditions without the antibody, as determined by thrombin generation assay (TGA). In some embodiments, the % Peak IIa is at least 99% in the presence of no less than 50 nM TF antibody compared to the control conditions without the antibody, as determined by thrombin generation assay (TGA).

In some embodiments, the % Peak IIa is at least 60% in the presence of no less than 10 nM TF antibody compared to the control conditions without the antibody, as determined by thrombin generation assay (TGA). In some embodiments, the % Peak IIa is at least 70% in the presence of no less than 10 nM TF antibody compared to the control conditions without the antibody, as determined by thrombin generation assay (TGA). In some embodiments, the % Peak IIa is at least 80% in the presence of no less than 10 nM TF antibody compared to the control conditions without the antibody, as determined by thrombin generation assay (TGA). In some embodiments, the % Peak IIa is at least 90% in the presence of no less than 10 nM TF antibody compared to the control conditions without the antibody, as determined by thrombin generation assay (TGA). In some embodiments, the % Peak IIa is at least 95% in the presence of no less than 10 nM TF antibody compared to the control conditions without the antibody, as determined by thrombin generation assay (TGA). In some embodiments, the % Peak IIa is at least 99% in the presence of no less than 10 nM TF antibody compared to the control conditions without the antibody, as determined by thrombin generation assay (TGA).

In some embodiments, the % Peak IIa in the presence of 100 nM TF antibody, as set forth in Table 6 and Table 37 is selected from about 99%, about 100%, about 103%, about 64%, about 52%, about 87%, about 96%, about 98%, and about 53% compared to the control conditions without the antibody, as determined by thrombin generation assay (TGA) without antibody pre-incubation. In some embodiments, such % Peak IIa ranges from about 52% to about 103%. In some embodiments, such % Peak IIa is about 52% or more.

In some embodiments, the % Peak IIa in the presence of 50 nM TF antibody, as set forth in Table 6 and Table 37 is selected from about 99%, about 100%, about 103%, about 67%, about 58%, about 89%, about 96%, about 98%, about 68%, about 62%, and about 88% compared to the control conditions without the antibody, as determined by thrombin generation assay (TGA) without antibody pre-incubation. In some embodiments, such % Peak IIa ranges from about 58% to about 103%. In some embodiments, such % Peak IIa is about 58% or more.

In some embodiments, the % Peak IIa in the presence of 10 nM TF antibody, as set forth in Table 6 and Table 37 is selected from about 100%, about 99%, about 103%, about 87%, about 83%, about 95%, about 98%, about 86%, and about 96% compared to the control conditions without the antibody, as determined by thrombin generation assay (TGA) without antibody pre-incubation. In some embodiments, such % Peak IIa ranges from about 83% to about 103%. In some embodiments, such % Peak IIa is about 83% or more.

In some embodiments, the % Peak IIa in the presence of 100 nM TF antibody, as set forth in Table 7 and Table 38 is selected from about 108%, about 105%, about 111%, about 58%, about 47%, about 91%, about 103%, about 109%, about 107%, and about 45% compared to the control conditions without the antibody, as determined by thrombin generation assay (TGA) with 10 min antibody pre-incubation. In some embodiments, such % Peak IIa ranges from about 45% to about 111%. In some embodiments, such % Peak IIa is about 45% or more.

In some embodiments, the % Peak IIa in the presence of 50 nM TF antibody, as set forth in Table 7 and Table 38 is selected from about 107%, about 104%, about 114%, about 62%, about 49%, about 87%, about 105%, about 109%, about 55%, and about 92% compared to the control conditions without the antibody, as determined by thrombin generation assay (TGA) with 10 min antibody pre-incubation. In some embodiments, such % Peak IIa ranges from about 49% to about 114%. In some embodiments, such % Peak IIa is about 49% or more.

In some embodiments, the % Peak IIa in the presence of 10 nM TF antibody, as set forth in Table 7 and Table 38 is selected from about 105%, about 114%, about 76%, about 68%, about 94%, about 108%, about 104%, about 74%, and about 93% compared to the control conditions without the antibody, as determined by thrombin generation assay (TGA) with 10 min antibody pre-incubation. In some embodiments, such % Peak IIa ranges from about 68% to about 114%. In some embodiments, such % Peak IIa is about 68% or more.

In some embodiments, the percent endogenous thrombin potential (% ETP) is at least 80% in the presence of no less than 100 nM TF antibody compared to the control conditions without the antibody, as determined by thrombin generation assay (TGA). In some embodiments, the % ETP is at least 90% in the presence of no less than 100 nM TF antibody compared to the control conditions without the antibody, as determined by thrombin generation assay (TGA). In some embodiments, the % ETP is at least 95% in the presence of no less than 100 nM TF antibody compared to the control conditions without the antibody, as determined by thrombin generation assay (TGA). In some embodiments, the % ETP is at least 99% in the presence of no less than 100 nM TF antibody compared to the control conditions without the antibody, as determined by thrombin generation assay (TGA).

In some embodiments, the % ETP is at least 80% in the presence of no less than 50 nM TF antibody compared to the control conditions without the antibody, as determined by thrombin generation assay (TGA). In some embodiments, the % ETP is at least 90% in the presence of no less than 50 nM TF antibody compared to the control conditions without the antibody, as determined by thrombin generation assay (TGA). In some embodiments, the % ETP is at least 95% in the presence of no less than 50 nM TF antibody compared to the control conditions without the antibody, as determined by thrombin generation assay (TGA). In some embodiments, the % ETP is at least 99% in the presence of no less than 50 nM TF antibody compared to the control conditions without the antibody, as determined by thrombin generation assay (TGA).

In some embodiments, the % ETP is at least 80% in the presence of no less than 10 nM TF antibody compared to the control conditions without the antibody, as determined by thrombin generation assay (TGA). In some embodiments, the % ETP is at least 90% in the presence of no less than 10 nM TF antibody compared to the control conditions without the antibody, as determined by thrombin generation assay (TGA). In some embodiments, the % ETP is at least 95% in the presence of no less than 10 nM TF antibody compared to the control conditions without the antibody, as determined by thrombin generation assay (TGA). In some embodiments, the % ETP is at least 99% in the presence of no less than 10 nM TF antibody compared to the control conditions without the antibody, as determined by thrombin generation assay (TGA).

In some embodiments, the % ETP in the presence of 100 nM TF antibody, as set forth in Table 6 and Table 37 is selected from about 108%, about 103%, about 109%, about 100%, about 96%, about 102%, about 105%, and about 92% compared to the control conditions without the antibody, as determined by thrombin generation assay (TGA) without antibody pre-incubation. In some embodiments, such % ETP ranges from about 92% to about 109%. In some embodiments, such % ETP is about 92% or more.

In some embodiments, the % ETP in the presence of 50 nM TF antibody, as set forth in Table 6 and Table 37 is selected from about 108%, about 103%, about 111%, about 101%, about 97%, about 104%, about 106%, about 93%, about 96%, and about 105% compared to the control conditions without the antibody, as determined by thrombin generation assay (TGA) without antibody pre-incubation. In some embodiments, such % ETP ranges from about 93% to about 111%. In some embodiments, such % ETP is about 93% or more.

In some embodiments, the % ETP in the presence of 10 nM TF antibody, as set forth in Table 6 and Table 37 is selected from about 106%, about 109%, about 105%, about 104%, about 107%, about 99%, about 101%, and about 102% compared to the control conditions without the antibody, as determined by thrombin generation assay (TGA) without antibody pre-incubation. In some embodiments, such % ETP ranges from about 99% to about 109%. In some embodiments, such % ETP is about 99% or more.

In some embodiments, the % ETP in the presence of 100 nM TF antibody, as set forth in Table 7 and Table 38 is selected from about 110%, about 104%, about 106%, about 98%, about 95%, about 108%, about 107%, about 96%, about 92%, and about 103% compared to the control conditions without the antibody, as determined by thrombin generation assay (TGA) with 10 min antibody pre-incubation. In some embodiments, such % ETP ranges from about 92% to about 110%. In some embodiments, such % ETP is about 92% or more.

In some embodiments, the % ETP in the presence of 50 nM TF antibody, as set forth in Table 7 and Table 38 is selected from about 110%, about 106%, about 108%, about 103%, about 96%, about 109%, about 102%, about 104%, about 94%, and about 98% compared to the control conditions without the antibody, as determined by thrombin generation assay (TGA) with 10 min antibody pre-incubation. In some embodiments, such % ETP ranges from about 94% to about 110%. In some embodiments, such % ETP is about 94% or more.

In some embodiments, the % ETP in the presence of 10 nM TF antibody, as set forth in Table 7 and Table 38 is selected from about 107%, about 106%, about 110%, about 103%, about 100%, about 105%, about 102%, and about 101% compared to the control conditions without the antibody, as determined by thrombin generation assay (TGA) with 10 min antibody pre-incubation. In some embodiments, such % ETP ranges from about 100% to about 110%. In some embodiments, such % ETP is about 100% or more.

### 2.3.3. FXa Conversion in the Presence of TF Antibodies

In some embodiments, the antibodies provided herein bind human TF at a human TF binding site that is distinct from a human TF binding site bound by human FX. In certain embodiments, the antibodies provided herein do not interfere with the ability of TF:FVIIa to convert FX into FXa.

In some embodiments, the percentage of FXa conversion (% FXa) is at least 75% in the presence of no less than 100 nM TF antibody compared to the control conditions without the antibody. In some embodiments, the % FXa is at least 80% in the presence of no less than 100 nM TF antibody compared to the control conditions without the antibody. In some embodiments, the % FXa is at least 85% in the presence of no less than 100 nM TF antibody compared to the control conditions without the antibody. In some embodiments, the % FXa is at least 90% in the presence of no less than 100 nM TF antibody compared to the control conditions without the antibody. In some embodiments, the % FXa is at least 95% in the presence of no less than 100 nM TF antibody compared to the control conditions without the antibody.

In some embodiments, the % FXa is at least 75% in the presence of no less than 50 nM TF antibody compared to the control conditions without the antibody. In some embodiments, the % FXa is at least 80% in the presence of no less than 50 nM TF antibody compared to the control conditions without the antibody. In some embodiments, the % FXa is at least 85% in the presence of no less than 50 nM TF antibody compared to the control conditions without the antibody. In some embodiments, the % FXa is at least 90% in the presence of no less than 50 nM TF antibody compared to the control conditions without the antibody. In some embodiments, the % FXa is at least 95% in the presence of no less than 50 nM TF antibody compared to the control conditions without the antibody.

In some embodiments, the % FXa is at least 75% in the presence of no less than 25 nM TF antibody compared to the control conditions without the antibody. In some embodiments, the % FXa is at least 80% in the presence of no less than 25 nM TF antibody compared to the control conditions without the antibody. In some embodiments, the % FXa is at least 85% in the presence of no less than 25 nM TF antibody compared to the control conditions without the antibody. In some embodiments, the % FXa is at least 90% in the presence of no less than 25 nM TF antibody compared to the control conditions without the antibody. In some embodiments, the % FXa is at least 95% in the presence of no less than 25 nM TF antibody compared to the control conditions without the antibody.

In some embodiments, the % FXa is at least 75% in the presence of no less than 12.5 nM TF antibody compared to the control conditions without the antibody. In some embodiments, the % FXa is at least 80% in the presence of no less than 12.5 nM TF antibody compared to the control conditions without the antibody. In some embodiments, the % FXa is at least 85% in the presence of no less than 12.5 nM TF antibody compared to the control conditions without the antibody. In some embodiments, % FXa is at least 90% in the presence of no less than 12.5 nM TF antibody compared to the control conditions without the antibody. In some embodiments, the % FXa is at least 95% in the presence of no less than 12.5 nM TF antibody compared to the control conditions without the antibody.

In some embodiments, the % FXa in the presence of 100 nM TF antibody, as set forth in Table 8 is selected from about 89%, about 96%, about 116%, about 108%, about 117%, about 105%, about 112%, about 106%, about 103%, about 111%, about 98%, and about 101% compared to the control conditions without the antibody. In some embodiments, such % FXa ranges from about 89% to about 117%. In some embodiments, such % FXa is about 89% or more.

In some embodiments, the % FXa in the presence of 50 nM TF antibody, as set forth in Table 8 is selected from about 94%, about 93%, about 78%, about 102%, about 99%, about 104%, about 105%, about 108%, about 107%, about 97%, and about 106% compared to the control conditions without the antibody. In some embodiments, such % FXa ranges from about 78% to about 108%. In some embodiments, such % FXa is about 78% or more.

In some embodiments, the % FXa in the presence of 25 nM TF antibody, as set forth in Table 8 is selected from about 81%, about 89%, about 85%, about 109%, about 96%, about 97%, about 108%, about 104%, about 103%, about 112%, and about 89% compared to the control conditions without the antibody. In some embodiments, such % FXa ranges from about 81% to about 112%. In some embodiments, such % FXa is about 81% or more.

In some embodiments, the % FXa in the presence of 12.5 nM TF antibody, as set forth in Table 8 is selected from about 87%, about 89%, about 82%, about 99%, about 101%, about 98%, about 113%, about 106%, about 115%, about 110%, about 120%, about 85%, and about 108% compared to the control conditions without the antibody. In some embodiments, such % FXa ranges from about 82% to about 120%. In some embodiments, such % FXa is about 82% or more.

### 2.3.4. FVIIa Binding in the Presence of TF Antibodies

In some embodiments, the antibodies provided herein bind human TF at a human TF binding site that is distinct from a human TF binding site bound by human FVIIa. In certain embodiments, the antibodies provided herein do not compete for binding to human TF with human FVIIa.

In some embodiments, the percentage of FVIIa binding (% FVIIa) is at least 75% in the presence of no less than 250 nM TF antibody compared to the control conditions without the antibody. In some embodiments, the % FVIIa is at least 80% in the presence of no less than 250 nM TF antibody compared to the control conditions without the antibody. In some embodiments, the % FVIIa is at least 85% in the presence of no less than 250 nM TF antibody compared to the control conditions without the antibody. In some embodiments, the % FVIIa is at least 90% in the presence of no less than 250 nM TF antibody compared to the control conditions without the antibody. In some embodiments, the % FVIIa is at least 95% in the presence of no less than 250 nM TF antibody compared to the control conditions without the antibody.

In some embodiments, the % FVIIa is at least 75% in the presence of no less than 83 nM TF antibody compared to the control conditions without the antibody. In some embodiments, the % FVIIa is at least 80% in the presence of no less than 83 nM TF antibody compared to the control conditions without the antibody. In some embodiments, the % FVIIa is at least 85% in the presence of no less than 83 nM TF antibody compared to the control conditions without the antibody. In some embodiments, the % FVIIa is at least 90% in the presence of no less than 83 nM TF antibody compared to the control conditions without the antibody. In some embodiments, the % FVIIa is at least 95% in the presence of no less than 83 nM TF antibody compared to the control conditions without the antibody.

In some embodiments, the % FVIIa is at least 75% in the presence of no less than 28 nM TF antibody compared to the control conditions without the antibody. In some embodiments, the % FVIIa is at least 80% in the presence of no less than 28 nM TF antibody compared to the control conditions without the antibody. In some embodiments, the % FVIIa is at least 85% in the presence of no less than 28 nM TF antibody compared to the control conditions without the antibody. In some embodiments, the % FVIIa is at least 90% in the presence of no less than 28 nM TF antibody compared to the control conditions without the antibody. In some embodiments, the % FVIIa is at least 95% in the presence of no less than 28 nM TF antibody compared to the control conditions without the antibody.

In some embodiments, the % FVIIa is at least 75% in the presence of no less than 9.25 nM TF antibody compared to the control conditions without the antibody. In some embodiments, the % FVIIa is at least 80% in the presence of no less than 9.25 nM TF antibody compared to the control conditions without the antibody. In some embodiments, the % FVIIa is at least 85% in the presence of no less than 9.25 nM TF antibody compared to the control conditions without the antibody. In some embodiments, the % FVIIa is at least 90% in the presence of no less than 9.25 nM TF antibody compared to the control conditions without the antibody. In some embodiments, the % FVIIa is at least 95% in the presence of no less than 9.25 nM TF antibody compared to the control conditions without the antibody.

In some embodiments, the % FVIIa in the presence of 250 nM TF antibody, as set forth in Table 9 is selected from about 98%, about 87%, about 80%, about 92%, about 95%, about 89%, about 91%, about 97%, about 94%, about 101%, and about 96% compared to the control conditions without the antibody. In some embodiments, such % FVIIa ranges from about 80% to about 101%. In some embodiments, such % FVIIa is about 80% or more.

In some embodiments, the % FVIIa in the presence of 83 nM TF antibody, as set forth in Table 9 is selected from about 97%, about 88%, about 77%, about 93%, about 94%, about 91%, about 98%, about 100%, and about 92% compared to the control conditions without the antibody. In some embodiments, such % FVIIa ranges from about 77% to about 100%. In some embodiments, such % FVIIa is about 77% or more.

In some embodiments, the % FVIIa in the presence of 28 nM TF antibody, as set forth in Table 9 is selected from about 101%, about 87%, about 79%, about 96%, about 93%, about 95%, about 98%, about 100%, about 102%, about 99%, about 92%, and about 91% compared to the control conditions without the antibody. In some embodiments, such % FVIIa ranges from about 79% to about 102%. In some embodiments, such % FVIIa is about 79% or more.

In some embodiments, the % FVIIa in the presence of 9.25 nM TF antibody, as set forth in Table 9 is selected from about 100%, about 90%, about 76%, about 97%, about 93%, about 99%, about 98%, about 102%, about 101%, and about 95% compared to the control conditions without the antibody. In some embodiments, such % FVIIa ranges from about 76% to about 102%. In some embodiments, such % FVIIa is about 76% or more.

### 2.3.5. FVIIa-Dependent TF Signaling in the Presence of TF Antibodies

In some embodiments, the antibodies provided herein inhibit FVIIa-dependent TF signaling. In some embodiments, the inhibition of FVIIa-dependent TF signaling is measured by the reduction of IL8. In some embodiments, the inhibition of FVIIa-dependent TF signaling is measured by the reduction of GM-CSF.

In some embodiments, the Interleukin 8 concentration (IL8 conc) is reduced by at least 70% in the presence of no less than 100 nM TF antibody compared to the control conditions without the antibody. In some embodiments, the IL8 conc is reduced by at least 80% in the presence of no less than 100 nM TF antibody compared to the control conditions without the antibody. In some embodiments, the IL8 conc is reduced by at least 90% in the presence of no less than 100 nM TF antibody compared to the control conditions without the antibody.

In some embodiments, the IL8 conc is reduced by at least 70% in the presence of no less than 40 nM TF antibody compared to the control conditions without the antibody. In some embodiments, the IL8 conc is reduced by at least 80% in the presence of no less than 40 nM TF antibody compared to the control conditions without the antibody. In some embodiments, the IL8 conc is reduced by at least 90% in the presence of no less than 40 nM TF antibody compared to the control conditions without the antibody.

In some embodiments, the IL8 conc is reduced by at least 60% in the presence of no less than 16 nM TF antibody compared to the control conditions without the antibody. In some embodiments, the IL8 conc is reduced by at least 70% in the presence of no less than 16 nM TF antibody compared to the control conditions without the antibody. In some embodiments, the IL8 conc is reduced by at least 80% in the presence of no less than 16 nM TF antibody compared to the control conditions without the antibody. In some embodiments, the IL8 conc is reduced by at least 90% in the presence of no less than 16 nM TF antibody compared to the control conditions without the antibody.

In some embodiments, the IL8 conc is reduced by at least 50% in the presence of no less than 6.4 nM TF antibody compared to the control conditions without the antibody. In some embodiments, the IL8 conc is reduced by at least 60% in the presence of no less than 6.4 nM TF antibody compared to the control conditions without the antibody. In some embodiments, the IL8 conc is reduced by at least 70% in the presence of no less than 6.4 nM TF antibody compared to the control conditions without the antibody. In some embodiments, the IL8 conc is reduced by at least 80% in the presence of no less than 6.4 nM TF antibody compared to the control conditions without the antibody. In some embodiments, the IL8 conc is reduced by at least 90% in the presence of no less than 6.4 nM TF antibody compared to the control conditions without the antibody.

In some embodiments, the Granulocyte-Macrophage Colony-Stimulating Factor concentration (GM-CSF conc) is reduced by at least 70% in the presence of no less than 100 nM TF antibody compared to the control conditions without the antibody. In some embodiments, the GM-CSF conc is reduced by at least 80% in the presence of no less than 100 nM TF antibody compared to the control conditions without the antibody. In some embodiments, the GM-CSF conc is reduced by at least 90% in the presence of no less than 100 nM TF antibody compared to the control conditions without the antibody.

In some embodiments, the GM-CSF conc is reduced by at least 70% in the presence of no less than 40 nM TF antibody compared to the control conditions without the antibody. In some embodiments, the GM-CSF conc is reduced by at least 80% in the presence of no less than 40 nM TF antibody compared to the control conditions without the antibody. In some embodiments, the GM-CSF conc is reduced by at least 90% in the presence of no less than 40 nM TF antibody compared to the control conditions without the antibody.

In some embodiments, the GM-CSF conc is reduced by at least 60% in the presence of no less than 16 nM TF antibody compared to the control conditions without the antibody. In some embodiments, the GM-CSF conc is reduced by at least 70% in the presence of no less than 16 nM TF antibody compared to the control conditions without the antibody. In some embodiments, the GM-CSF conc is reduced by at least 80% in the presence of no less than 16 nM TF antibody compared to the control conditions without the antibody. In some embodiments, the GM-CSF conc is reduced by at least 90% in the presence of no less than 16 nM TF antibody compared to the control conditions without the antibody.

In some embodiments, the GM-CSF conc is reduced by at least 50% in the presence of no less than 6.4 nM TF antibody compared to the control conditions without the antibody. In some embodiments, the GM-CSF conc is reduced by at least 60% in the presence of no less than 6.4 nM TF antibody compared to the control conditions without the antibody. In some embodiments, the GM-CSF conc is reduced by at least 70% in the presence of no less than 6.4 nM TF antibody compared to the control conditions without the antibody. In some embodiments, the GM-CSF conc is reduced by at least 80% in the presence of no less than 6.4 nM TF antibody compared to the control conditions without the antibody. In some embodiments, the GM-CSF conc is reduced by at least 90% in the presence of no less than 6.4 nM TF antibody compared to the control conditions without the antibody.

In some embodiments, the percentage of Interleukin 8 (% IL8) in the presence of 100 nM TF antibody, as set forth in Table 10 is selected from about 2%, about 9%, about 8%, about 6%, about 13%, about 1%, about 3%, about 4%, and about 5% compared to the control conditions without the antibody. In some embodiments, such % IL8 ranges from about 1% to about 13%. In some embodiments, such % IL8 is about 13% or less.

In some embodiments, the % IL8 in the presence of 40 nM TF antibody, as set forth in Table 10 is selected from about 2%, about 8%, about 7%, about 10%, about 14%, about 4%, about 5%, and about 6% compared to the control conditions without the antibody. In some embodiments, such % IL8 ranges from about 2% to about 14%. In some embodiments, such % IL8 is about 14% or less.

In some embodiments, the % IL8 in the presence of 16 nM TF antibody, as set forth in Table 10 is selected from about 2%, about 3%, about 10%, about 8%, about 7%, about 16%, about 9%, about 15%, about 5%, and about 6% compared to the control conditions without the antibody. In some embodiments, such % IL8 ranges from about 2% to about 16%. In some embodiments, such % IL8 is about 16% or less.

In some embodiments, the % IL8 in the presence of 6.4 nM TF antibody, as set forth in Table 10 is selected from about 3%, about 4%, about 11%, about 9%, about 14%, about 22%, about 12%, about 6%, about 5%, about 15%, about 21%, and about 8% compared to the control conditions without the antibody. In some embodiments, such % IL8 ranges from about 3% to about 22%. In some embodiments, such % IL8 is about 22% or less.

In some embodiments, the percentage of Granulocyte-Macrophage Colony-Stimulating Factor (% GM-CSF) in the presence of 100 nM TF antibody, as set forth in Table 11 is selected from about 6%, about 7%, about 22%, about 20%, about 12%, about 19%, about 17%, about 25%, about 5%, about 14%, about 11%, and about 10% compared to the control conditions without the antibody. In some embodiments, such % GM-CSF ranges from about 5% to about 25%. In some embodiments, such % GM-CSF is about 25% or less.

In some embodiments, the % GM-CSF in the presence of 40 nM TF antibody, as set forth in Table 11 is selected from about 6%, about 7%, about 19%, about 15%, about 18%, about 16%, about 26%, about 5%, about 13%, about 11%, and about 10% compared to the control conditions without the antibody. In some embodiments, such % GM-CSF ranges from about 5% to about 26%. In some embodiments, such % GM-CSF is about 26% or less.

In some embodiments, the % GM-CSF in the presence of 16 nM TF antibody, as set forth in Table 11 is selected from about 6%, about 7%, about 22%, about 19%, about 14%, about 32%, about 17%, about 26%, about 5%, about 12%, about 13%, about 9%, about 11%, and about 15% compared to the control conditions without the antibody. In some embodiments, such % GM-CSF ranges from about 5% to about 32%. In some embodiments, such % GM-CSF is about 32% or less.

In some embodiments, the % GM-CSF in the presence of 6.4 nM TF antibody, as set forth in Table 11 is selected from about 8%, about 9%, about 24%, about 20%, about 18%, about 39%, about 34%, about 15%, about 21%, about 16%, about 17%, and about 10% compared to the control conditions without the antibody. In some embodiments, such % GM-CSF ranges from about 8% to about 39%. In some embodiments, such % GM-CSF is about 39% or less.

### 2.3.6. Lesion Size Reduction in Swine Choroidal Neovascularization (CNV) Model

In some embodiments, the antibodies provided herein reduce lesion size in a swine choroidal neovascularization (CNV) model. In some embodiments, the reduction in lesion size is measured by Fluorescein Angiography (FA).

In some embodiments, the lesion size in a swine CNV model is reduced by at least 5% 7 days after administration of the anti-TF antibody. In some embodiments, the lesion size in a swine CNV model is reduced by at least 10% 7 days after administration of the anti-TF antibody. In some embodiments, the lesion size in a swine CNV model is reduced by at least 20% 7 days after administration of the anti-TF antibody. In some embodiments, the lesion size in a swine CNV model is reduced by at least 40% 7 days after administration of the anti-TF antibody. In some embodiments, the lesion size in a swine CNV model is reduced by at least 60% 7 days after administration of the anti-TF antibody.

In some embodiments, the lesion size in a swine CNV model is reduced by at least 10% 21 days after administration of the anti-TF antibody. In some embodiments, the lesion size in a swine CNV model is reduced by at least 20% 21 days after administration of the anti-TF antibody. In some embodiments, the lesion size in a swine CNV model is reduced by at least 40% 21 days after administration of the anti-TF antibody. In some embodiments, the lesion size in a swine CNV model is reduced by at least 60% 21 days after administration of the anti-TF antibody. In some embodiments, the lesion size in a swine CNV model is reduced by at least 80% 21 days after administration of the anti-TF antibody.

### 2.4. Germlines

The antibodies provided herein may comprise any suitable V_{H} and V_{L} germline sequences.

In some embodiments, the V_{H} region of an antibody provided herein is from the VH3 germline. In some embodiments, the V_{H} region of an antibody provided herein is from the VH1 germline. In some embodiments, the V_{H} region of an antibody provided herein is from the VH4 germline.

In some embodiments, the V_{H} region of an antibody provided herein is from the VH3-23 germline. In some embodiments, the V_{H} region of an antibody provided herein is from the VH1-18 germline. In some embodiments, the V_{H} region of an antibody provided herein is from the VH3-30 germline. In some embodiments, the V_{H} region of an antibody provided herein is from the VH1-69 germline. In some embodiments, the V_{H} region of an antibody provided herein is from the VH4-31 germline. In some embodiments, the V_{H} region of an antibody provided herein is from the VH4-34 germline. In some embodiments, the V_{H} region of an antibody provided herein is from the VH1-46 germline.

In some embodiments, the VL region of an antibody provided herein is from the VK1 germline. In some embodiments, the VL region of an antibody provided herein is from the VK4 germline. In some embodiments, the VL region of an antibody provided herein is from the VK3 germline

In some embodiments, the VL region of an antibody provided herein is from the VK1-05 germline. In some embodiments, the VL region of an antibody provided herein is from the VK4-01 germline. In some embodiments, the VL region of an antibody provided herein is from the VK3-15 germline. In some embodiments, the VL region of an antibody provided herein is from the VK3-20 germline. In some embodiments, the VL region of an antibody provided herein is from the VK1-33 germline.

### 2.5. Monospecific and Multispecific TF Antibodies

In some embodiments, the antibodies provided herein are monospecific antibodies.

In some embodiments, the antibodies provided herein are multispecific antibodies.

In some embodiments, a multispecific antibody provided herein binds more than one antigen. In some embodiments, a multispecific antibody binds two antigens. In some embodiments, a multispecific antibody binds three antigens. In some embodiments, a multispecific antibody binds four antigens. In some embodiments, a multispecific antibody binds five antigens.

In some embodiments, a multispecific antibody provided herein binds more than one epitope on a TF antigen. In some embodiments, a multispecific antibody binds two epitopes on a TF antigen. In some embodiments, a multispecific antibody binds three epitopes on a TF antigen.

Many multispecific antibody constructs are known in the art, and the antibodies provided herein may be provided in the form of any suitable multispecific suitable construct.

In some embodiments, the multispecific antibody comprises an immunoglobulin comprising at least two different heavy chain variable regions each paired with a common light chain variable region (i.e., a “common light chain antibody”). The common light chain variable region forms a distinct antigen-binding domain with each of the two different heavy chain variable regions. See Merchant et al., Nature Biotechnol., 1998, 16:677-681, incorporated by reference in its entirety.

In some embodiments, the multispecific antibody comprises an immunoglobulin comprising an antibody or fragment thereof attached to one or more of the N- or C-termini of the heavy or light chains of such immunoglobulin. See Coloma and Morrison, Nature Biotechnol., 1997, 15:159-163, incorporated by reference in its entirety. In some aspects, such antibody comprises a tetravalent bispecific antibody.

In some embodiments, the multispecific antibody comprises a hybrid immunoglobulin comprising at least two different heavy chain variable regions and at least two different light chain variable regions. See Milstein and Cuello, Nature, 1983, 305:537-540; and Staerz and Bevan, Proc. Natl. Acad. Sci. USA, 1986, 83:1453-1457; each of which is incorporated by reference in its entirety.

In some embodiments, the multispecific antibody comprises immunoglobulin chains with alterations to reduce the formation of side products that do not have multispecificity. In some aspects, the antibodies comprise one or more “knobs-into-holes” modifications as described in U.S. Pat. No. 5,731,168, incorporated by reference in its entirety.

In some embodiments, the multispecific antibody comprises immunoglobulin chains with one or more electrostatic modifications to promote the assembly of Fc hetero-multimers. See WO 2009/089004, incorporated by reference in its entirety.

In some embodiments, the multispecific antibody comprises a bispecific single chain molecule. See Traunecker et al., EMBO J., 1991, 10:3655-3659; and Gruber et al., J. Immunol., 1994, 152:5368-5374; each of which is incorporated by reference in its entirety.

In some embodiments, the multispecific antibody comprises a heavy chain variable domain and a light chain variable domain connected by a polypeptide linker, where the length of the linker is selected to promote assembly of multispecific antibodies with the desired multispecificity. For example, monospecific scFvs generally form when a heavy chain variable domain and light chain variable domain are connected by a polypeptide linker of more than 12 amino acid residues. See U.S. Pat. Nos. 4,946,778 and 5,132,405, each of which is incorporated by reference in its entirety. In some embodiments, reduction of the polypeptide linker length to less than 12 amino acid residues prevents pairing of heavy and light chain variable domains on the same polypeptide chain, thereby allowing pairing of heavy and light chain variable domains from one chain with the complementary domains on another chain. The resulting antibodies therefore have multispecificity, with the specificity of each binding site contributed by more than one polypeptide chain. Polypeptide chains comprising heavy and light chain variable domains that are joined by linkers between 3 and 12 amino acid residues form predominantly dimers (termed diabodies). With linkers between 0 and 2 amino acid residues, trimers (termed triabodies) and tetramers (termed tetrabodies) are favored. However, the exact type of oligomerization appears to depend on the amino acid residue composition and the order of the variable domain in each polypeptide chain (e.g., V_{H}-linker-V_{L} VS. V_{L}-linker-V_{H}), in addition to the linker length. A skilled person can select the appropriate linker length based on the desired multispecificity.

In some embodiments, the multispecific antibody comprises a diabody. See Hollinger et al., Proc. Natl. Acad. Sci. USA, 1993, 90:6444-6448, incorporated by reference in its entirety. In some embodiments, the multispecific antibody comprises a triabody. See Todorovska et al., J. Immunol. Methods, 2001, 248:47-66, incorporated by reference in its entirety. In some embodiments, the multispecific antibody comprises a tetrabody. See id., incorporated by reference in its entirety.

In some embodiments, the multispecific antibody comprises a trispecific F(ab′)3 derivative. See Tutt et al. J. Immunol., 1991, 147:60-69, incorporated by reference in its entirety.

In some embodiments, the multispecific antibody comprises a cross-linked antibody. See U.S. Pat. No. 4,676,980; Brennan et al., Science, 1985, 229:81-83; Staerz, et al. Nature, 1985, 314:628-631; and EP 0453082; each of which is incorporated by reference in its entirety.

In some embodiments, the multispecific antibody comprises antigen-binding domains assembled by leucine zippers. See Kostelny et al., J. Immunol., 1992, 148:1547-1553, incorporated by reference in its entirety.

In some embodiments, the multispecific antibody comprises complementary protein domains. In some aspects, the complementary protein domains comprise an anchoring domain (AD) and a dimerization and docking domain (DDD). In some embodiments, the AD and DDD bind to each other and thereby enable assembly of multispecific antibody structures via the “dock and lock” (DNL) approach. Antibodies of many specificities may be assembled, including bispecific antibodies, trispecific antibodies, tetraspecific antibodies, quintspecific antibodies, and hexaspecific antibodies. Multispecific antibodies comprising complementary protein domains are described, for example, in U.S. Pat. Nos. 7,521,056; 7,550,143; 7,534,866; and 7,527,787; each of which is incorporated by reference in its entirety.

In some embodiments, the multispecific antibody comprises a dual action Fab (DAF) antibody as described in U.S. Pat. Pub. No. 2008/0069820, incorporated by reference in its entirety.

In some embodiments, the multispecific antibody comprises an antibody formed by reduction of two parental molecules followed by mixing of the two parental molecules and reoxidation to assembly a hybrid structure. See Carlring et al., PLoS One, 2011, 6:e22533, incorporated by reference in its entirety.

In some embodiments, the multispecific antibody comprises a DVD-Ig™. A DVD-Ig™ is a dual variable domain immunoglobulin that can bind to two or more antigens. DVD-Igs™ are described in U.S. Pat. No. 7,612,181, incorporated by reference in its entirety.

In some embodiments, the multispecific antibody comprises a DART™. DARTs™ are described in Moore et al., Blood, 2011, 117:454-451, incorporated by reference in its entirety.

In some embodiments, the multispecific antibody comprises a DuoBody®. DuoBodies® are described in Labrijn et al., Proc. Natl. Acad. Sci. USA, 2013, 110:5145-5150; Gramer et al., mAbs, 2013, 5:962-972; and Labrijn et al., Nature Protocols, 2014, 9:2450-2463; each of which is incorporated by reference in its entirety.

In some embodiments, the multispecific antibody comprises an antibody fragment attached to another antibody or fragment. The attachment can be covalent or non-covalent. When the attachment is covalent, it may be in the form of a fusion protein or via a chemical linker. Illustrative examples of multispecific antibodies comprising antibody fragments attached to other antibodies include tetravalent bispecific antibodies, where an scFv is fused to the C-terminus of the C_{H3} from an IgG. See Coloma and Morrison, Nature Biotechnol., 1997, 15:159-163. Other examples include antibodies in which a Fab molecule is attached to the constant region of an immunoglobulin. See Miler et al., J. Immunol., 2003, 170:4854-4861, incorporated by reference in its entirety. Any suitable fragment may be used, including any of the fragments described herein or known in the art.

In some embodiments, the multispecific antibody comprises a CovX-Body. CovX-Bodies are described, for example, in Doppalapudi et al., Proc. Natl. Acad. Sci. USA, 2010, 107:22611-22616, incorporated by reference in its entirety.

In some embodiments, the multispecific antibody comprises an Fcab antibody, where one or more antigen-binding domains are introduced into an Fc region. Fcab antibodies are described in Wozniak-Knopp et al., Protein Eng. Des. Sel., 2010, 23:289-297, incorporated by reference in its entirety.

In some embodiments, the multispecific antibody comprises a TandAb® antibody. TandAb® antibodies are described in Kipriyanov et al., J. Mol. Biol., 1999, 293:41-56 and Zhukovsky et al., Blood, 2013, 122:5116, each of which is incorporated by reference in its entirety.

In some embodiments, the multispecific antibody comprises a tandem Fab. Tandem Fabs are described in WO 2015/103072, incorporated by reference in its entirety.

In some embodiments, the multispecific antibody comprises a Zybody™ Zybodies™ are described in LaFleur et al., mAbs, 2013, 5:208-218, incorporated by reference in its entirety.

### 2.6. Glycosylation Variants

In certain embodiments, an antibody provided herein may be altered to increase, decrease or eliminate the extent to which it is glycosylated. Glycosylation of polypeptides is typically either “N-linked” or “O-linked.”

“N-linked” glycosylation refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site.

“O-linked” glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition or deletion of N-linked glycosylation sites to or from an antibody provided herein may be accomplished by altering the amino acid sequence such that one or more of the above-described tripeptide sequences is created or removed. Addition or deletion of O-linked glycosylation sites may be accomplished by addition, deletion, or substitution of one or more serine or threonine residues in or to (as the case may be) the sequence of an antibody.

In some embodiments, an antibody provided herein comprises a glycosylation motif that is different from a naturally occurring antibody. Any suitable naturally occurring glycosylation motif can be modified in the antibodies provided herein. The structural and glycosylation properties of immunoglobulins, for example, are known in the art and summarized, for example, in Schroeder and Cavacini, J. Allergy Clin. Immunol., 2010, 125:S41-52, incorporated by reference in its entirety.

In some embodiments, an antibody provided herein comprises an IgG1 Fc region with modification to the oligosaccharide attached to asparagine 297 (Asn 297). Naturally occurring IgG1 antibodies produced by mammalian cells typically comprise a branched, biantennary oligosaccharide that is generally attached by an N-linkage to Asn 297 of the C_{H2} domain of the Fc region. See Wright et al., TIBTECH, 1997, 15:26-32, incorporated by reference in its entirety. The oligosaccharide attached to Asn 297 may include various carbohydrates such as mannose, N-acetyl glucosamine (GlcNAc), galactose, and sialic acid, as well as a fucose attached to a GlcNAc in the “stem” of the biantennary oligosaccharide structure.

In some embodiments, the oligosaccharide attached to Asn 297 is modified to create antibodies having altered ADCC. In some embodiments, the oligosaccharide is altered to improve ADCC. In some embodiments, the oligosaccharide is altered to reduce ADCC.

In some aspects, an antibody provided herein comprises an IgG1 domain with reduced fucose content at position Asn 297 compared to a naturally occurring IgG1 domain. Such Fc domains are known to have improved ADCC. See Shields et al., J. Biol. Chem., 2002, 277:26733-26740, incorporated by reference in its entirety. In some aspects, such antibodies do not comprise any fucose at position Asn 297. The amount of fucose may be determined using any suitable method, for example as described in WO 2008/077546, incorporated by reference in its entirety.

In some embodiments, an antibody provided herein comprises a bisected oligosaccharide, such as a biantennary oligosaccharide attached to the Fc region of the antibody that is bisected by GlcNAc. Such antibody variants may have reduced fucosylation and/or improved ADCC function. Examples of such antibody variants are described, for example, in WO 2003/011878; U.S. Pat. No. 6,602,684; and U.S. Pat. Pub. No. 2005/0123546; each of which is incorporated by reference in its entirety.

Other illustrative glycosylation variants which may be incorporated into the antibodies provided herein are described, for example, in U.S. Pat. Pub. Nos. 2003/0157108, 2004/0093621, 2003/0157108, 2003/0115614, 2002/0164328, 2004/0093621, 2004/0132140, 2004/0110704, 2004/0110282, 2004/0109865; International Pat. Pub. Nos. 2000/61739, 2001/29246, 2003/085119, 2003/084570, 2005/035586, 2005/035778; 2005/053742, 2002/031140; Okazaki et al., J. Mol. Biol., 2004, 336:1239-1249; and Yamane-Ohnuki et al., Biotech. Bioeng., 2004, 87: 614-622; each of which is incorporated by reference in its entirety.

In some embodiments, an antibody provided herein comprises an Fc region with at least one galactose residue in the oligosaccharide attached to the Fc region. Such antibody variants may have improved CDC function. Examples of such antibody variants are described, for example, in WO 1997/30087; WO 1998/58964; and WO 1999/22764; each of which is incorporated by reference in its entirety.

Examples of cell lines capable of producing defucosylated antibodies include Lec13 CHO cells, which are deficient in protein fucosylation (see Ripka et al., Arch. Biochem. Biophys., 1986, 249:533-545; U.S. Pat. Pub. No. 2003/0157108; WO 2004/056312; each of which is incorporated by reference in its entirety), and knockout cell lines, such as alpha-1,6-fucosyltransferase gene or FUT8 knockout CHO cells (see Yamane-Ohnuki et al., Biotech. Bioeng., 2004, 87: 614-622; Kanda et al., Biotechnol. Bioeng., 2006, 94:680-688; and WO 2003/085107; each of which is incorporated by reference in its entirety).

In some embodiments, an antibody provided herein is an aglycosylated antibody. An aglycosylated antibody can be produced using any method known in the art or described herein. In some aspects, an aglycosylated antibody is produced by modifying the antibody to remove all glycosylation sites. In some aspects, the glycosylation sites are removed only from the Fc region of the antibody. In some aspects, an aglycosylated antibody is produced by expressing the antibody in an organism that is not capable of glycosylation, such as E. coli, or by expressing the antibody in a cell-free reaction mixture.

In some embodiments, an antibody provided herein has a constant region with reduced effector function compared to a native IgG1 antibody. In some embodiments, the affinity of a constant region of an Fc region of an antibody provided herein for Fc receptor is less than the affinity of a native IgG1 constant region for such Fc receptor.

### 2.7. Fc Region Amino Acid Sequence Variants

In certain embodiments, an antibody provided herein comprises an Fc region with one or more amino acid substitutions, insertions, or deletions in comparison to a naturally occurring Fc region. In some aspects, such substitutions, insertions, or deletions yield antibodies with altered stability, glycosylation, or other characteristics. In some aspects, such substitutions, insertions, or deletions yield aglycosylated antibodies.

In some aspects, the Fc region of an antibody provided herein is modified to yield an antibody with altered affinity for an Fc receptor, or an antibody that is more immunologically inert. In some embodiments, the antibody variants provided herein possess some, but not all, effector functions. Such antibodies may be useful, for example, when the half-life of the antibody is important in vivo, but when certain effector functions (e.g., complement activation and ADCC) are unnecessary or deleterious.

In some embodiments, the Fc region of an antibody provided herein is a human IgG4 Fc region comprising one or more of the hinge stabilizing mutations S228P and L235E. See Aalberse et al., Immunology, 2002, 105:9-19, incorporated by reference in its entirety. In some embodiments, the IgG4 Fc region comprises one or more of the following mutations: E233P, F234V, and L235A. See Armour et al., Mol. Immunol., 2003, 40:585-593, incorporated by reference in its entirety. In some embodiments, the IgG4 Fc region comprises a deletion at position G236.

In some embodiments, the Fc region of an antibody provided herein is a human IgG1 Fc region comprising one or more mutations to reduce Fc receptor binding. In some aspects, the one or more mutations are in residues selected from S228 (e.g., S228A), L234 (e.g., L234A), L235 (e.g., L235A), D265 (e.g., D265A), and N297 (e.g., N297A). In some aspects, the antibody comprises a PVA236 mutation. PVA236 means that the amino acid sequence ELLG (SEQ ID NO:920), from amino acid position 233 to 236 of IgG1 or EFLG (SEQ ID NO:921) of IgG4, is replaced by PVA. See U.S. Pat. No. 9,150,641, incorporated by reference in its entirety.

In some embodiments, the Fc region of an antibody provided herein is modified as described in Armour et al., Eur. J. Immunol., 1999, 29:2613-2624; WO 1999/058572; and/or U.K. Pat. App. No. 98099518; each of which is incorporated by reference in its entirety.

In some embodiments, the Fc region of an antibody provided herein is a human IgG2 Fc region comprising one or more of mutations A330S and P331S.

In some embodiments, the Fc region of an antibody provided herein has an amino acid substitution at one or more positions selected from 238, 265, 269, 270, 297, 327 and 329. See U.S. Pat. No. 6,737,056, incorporated by reference in its entirety. Such Fc mutants include Fc mutants with substitutions at two or more of amino acid positions 265, 269, 270, 297 and 327, including the so-called “DANA” Fc mutant with substitution of residues 265 and 297 with alanine. See U.S. Pat. No. 7,332,581, incorporated by reference in its entirety. In some embodiments, the antibody comprises an alanine at amino acid position 265. In some embodiments, the antibody comprises an alanine at amino acid position 297.

In certain embodiments, an antibody provided herein comprises an Fc region with one or more amino acid substitutions which improve ADCC, such as a substitution at one or more of positions 298, 333, and 334 of the Fc region. In some embodiments, an antibody provided herein comprises an Fc region with one or more amino acid substitutions at positions 239, 332, and 330, as described in Lazar et al., Proc. Natl. Acad. Sci. USA, 2006, 103:4005-4010, incorporated by reference in its entirety.

In some embodiments, an antibody provided herein comprises one or more alterations that improves or diminishes C1q binding and/or CDC. See U.S. Pat. No. 6,194,551; WO 99/51642; and Idusogie et al., J. Immunol., 2000, 164:4178-4184; each of which is incorporated by reference in its entirety.

In some embodiments, an antibody provided herein comprises one or more alterations to increase half-life. Antibodies with increased half-lives and improved binding to the neonatal Fc receptor (FcRn) are described, for example, in Hinton et al., J. Immunol., 2006, 176:346-356; and U.S. Pat. Pub. No. 2005/0014934; each of which is incorporated by reference in its entirety. Such Fc variants include those with substitutions at one or more of Fc region residues: 238, 250, 256, 265, 272, 286, 303, 305, 307, 311, 312, 314, 317, 340, 356, 360, 362, 376, 378, 380, 382, 413, 424, 428, and 434 of an IgG.

In some embodiments, an antibody provided herein comprises one or more Fc region variants as described in U.S. Pat. Nos. 7,371,826, 5,648,260, and 5,624,821; Duncan and Winter, Nature, 1988, 322:738-740; and WO 94/29351; each of which is incorporated by reference in its entirety.

### 2.8. Pyroglutamate

As is known in the art, both glutamate (E) and glutamine (Q) at the N-termini of recombinant proteins can cyclize spontaneously to form pyroglutamate (pE) in vitro and in vivo. See Liu et al., J. Biol. Chem., 2011, 286:11211-11217, incorporated by reference in its entirety.

In some embodiments, provided herein are antibodies comprising a polypeptide sequence having a pE residue at the N-terminal position. In some embodiments, provided herein are antibodies comprising a polypeptide sequence in which the N-terminal residue has been converted from Q to pE. In some embodiments, provided herein are antibodies comprising a polypeptide sequence in which the N-terminal residue has been converted from E to pE.

### 2.9. Cysteine Engineered Antibody Variants

In certain embodiments, provided herein are cysteine engineered antibodies, also known as “thioMAbs,” in which one or more residues of the antibody are substituted with cysteine residues. In particular embodiments, the substituted residues occur at solvent accessible sites of the antibody. By substituting such residues with cysteine, reactive thiol groups are introduced at solvent accessible sites of the antibody and may be used to conjugate the antibody to other moieties, such as drug moieties or linker-drug moieties, for example, to create an immunoconjugate.

In certain embodiments, any one or more of the following residues may be substituted with cysteine: V205 of the light chain; A118 of the heavy chain Fc region; and S400 of the heavy chain Fc region. Cysteine engineered antibodies may be generated as described, for example, in U.S. Pat. No. 7,521,541, which is incorporated by reference in its entirety.

### 3. Anti-TF Antibody-Drug Conjugates

Provided herein are antibody-drug conjugates (ADCs) comprising an antibody that binds specifically to TF and a cytotoxic agent. In some embodiments, the cytotoxic agent is linked directly to the anti-TF antibody. In some embodiments, the cytotoxic agent is linked indirectly to the anti-TF antibody.

In some embodiments, the ADCs further comprise a linker. In some embodiments, the linker links the anti-TF antibody to the cytotoxic agent.

In some embodiments, the ADCs provided herein have a drug-antibody ratio (DAR) of 1. In some embodiments, the ADCs provided herein have a DAR of 2. In some embodiments, the ADCs provided herein have a DAR of 3. In some embodiments, the ADCs provided herein have a DAR of 4. In some embodiments, the ADCs provided herein have a DAR of 5. In some embodiments, the ADCs provided herein have a DAR of 1-2, 1-3, 1-4, 1-5, 2-3, 2-4, 2-5, 3-4, 3-5, 4-5, 1, 2, 3, 4, or 5. In some embodiments, the ADCs provided herein have a DAR greater than 5. In some embodiments, the DAR is measured by UV/vis spectroscopy, hydrophobic interaction chromatography (HIC), and/or reverse phase liquid chromatography separation with time-of-flight detection and mass characterization (RP-UPLC/Mass spectrometry).

### 4. Methods for Making TF Antibodies

### 4.1. TF Antigen Preparation

The TF antigen used for isolation of the antibodies provided herein may be intact TF or a fragment of TF. The TF antigen may be, for example, in the form of an isolated protein or a protein expressed on the surface of a cell.

In some embodiments, the TF antigen is a non-naturally occurring variant of TF, such as a TF protein having an amino acid sequence or post-translational modification that does not occur in nature.

In some embodiments, the TF antigen is truncated by removal of, for example, intracellular or membrane-spanning sequences, or signal sequences. In some embodiments, the TF antigen is fused at its C-terminus to a human IgG1 Fc domain or a polyhistidine tag.

### 4.2. Methods of Making Monoclonal Antibodies

Monoclonal antibodies may be obtained, for example, using the hybridoma method first described by Kohler et al., Nature, 1975, 256:495-497 (incorporated by reference in its entirety), and/or by recombinant DNA methods (see e.g., U.S. Pat. No. 4,816,567, incorporated by reference in its entirety). Monoclonal antibodies may also be obtained, for example, using phage-display libraries (see e.g., U.S. Pat. No. 8,258,082, which is incorporated by reference in its entirety) or, alternatively, using yeast-based libraries (see e.g., U.S. Pat. No. 8,691,730, which is incorporated by reference in its entirety).

In the hybridoma method, a mouse or other appropriate host animal is immunized to elicit lymphocytes that produce or are capable of producing antibodies that will specifically bind to the protein used for immunization. Alternatively, lymphocytes may be immunized in vitro. Lymphocytes are then fused with myeloma cells using a suitable fusing agent, such as polyethylene glycol, to form a hybridoma cell. See Goding J. W., Monoclonal Antibodies: Principles and Practice 3^{rd} ed. (1986) Academic Press, San Diego, CA, incorporated by reference in its entirety.

The hybridoma cells are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of antibody by the selected antibody-producing cells, and are sensitive media conditions, such as the presence or absence of HAT medium. Among these, preferred myeloma cell lines are murine myeloma lines, such as those derived from MOP-21 and MC-11 mouse tumors (available from the Salk Institute Cell Distribution Center, San Diego, CA), and SP-2 or X63-Ag8-653 cells (available from the American Type Culture Collection, Rockville, MD). Human myeloma and mouse-human heteromyeloma cell lines also have been described for the production of human monoclonal antibodies. See e.g., Kozbor, J. Immunol., 1984, 133:3001, incorporated by reference in its entirety.

After the identification of hybridoma cells that produce antibodies of the desired specificity, affinity, and/or biological activity, selected clones may be subcloned by limiting dilution procedures and grown by standard methods. See Goding, supra. Suitable culture media for this purpose include, for example, D-MEM or RPMI-1640 medium. In addition, the hybridoma cells may be grown in vivo as ascites tumors in an animal.

DNA encoding the monoclonal antibodies may be readily isolated and sequenced using conventional procedures (e.g., by using oligonucleotide probes that are capable of binding specifically to genes encoding the heavy and light chains of the monoclonal antibodies). Thus, the hybridoma cells can serve as a useful source of DNA encoding antibodies with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce antibody, to produce the monoclonal antibodies.

### 4.3. Methods of Making Chimeric Antibodies

Illustrative methods of making chimeric antibodies are described, for example, in U.S. Pat. No. 4,816,567; and Morrison et al., Proc. Natl. Acad Sci. USA, 1984, 81:6851-6855; each of which is incorporated by reference in its entirety. In some embodiments, a chimeric antibody is made by using recombinant techniques to combine a non-human variable region (e.g., a variable region derived from a mouse, rat, hamster, rabbit, or non-human primate, such as a monkey) with a human constant region.

### 4.4. Methods of Making Humanized Antibodies

Humanized antibodies may be generated by replacing most, or all, of the structural portions of a non-human monoclonal antibody with corresponding human antibody sequences. Consequently, a hybrid molecule is generated in which only the antigen-specific variable, or CDR, is composed of non-human sequence. Methods to obtain humanized antibodies include those described in, for example, Winter and Milstein, Nature, 1991, 349:293-299; Rader et al., Proc. Nat. Acad Sci. U.S.A., 1998, 95:8910-8915; Steinberger et al., J. Biol. Chem., 2000, 275:36073-36078; Queen et al., Proc. Natl. Acad Sci. U.S.A., 1989, 86:10029-10033; and U.S. Pat. Nos. 5,585,089, 5,693,761, 5,693,762, and 6,180,370; each of which is incorporated by reference in its entirety.

### 4.5. Methods of Making Human Antibodies

Human antibodies can be generated by a variety of techniques known in the art, for example by using transgenic animals (e.g., humanized mice). See, e.g., Jakobovits et al., Proc. Natl. Acad Sci. U.S.A., 1993, 90:2551; Jakobovits et al., Nature, 1993, 362:255-258; Bruggermann et al., Year in Immunol., 1993, 7:33; and U.S. Pat. Nos. 5,591,669, 5,589,369 and 5,545,807; each of which is incorporated by reference in its entirety. Human antibodies can also be derived from phage-display libraries (see e.g., Hoogenboom et al., J. Mol. Biol., 1991, 227:381-388; Marks et al., J. Mol. Biol., 1991, 222:581-597; and U.S. Pat. Nos. 5,565,332 and 5,573,905; each of which is incorporated by reference in its entirety). Human antibodies may also be generated by in vitro activated B cells (see e.g., U.S. Pat. Nos. 5,567,610 and 5,229,275, each of which is incorporated by reference in its entirety). Human antibodies may also be derived from yeast-based libraries (see e.g., U.S. Pat. No. 8,691,730, incorporated by reference in its entirety).

### 4.6. Methods of Making Antibody Fragments

The antibody fragments provided herein may be made by any suitable method, including the illustrative methods described herein or those known in the art. Suitable methods include recombinant techniques and proteolytic digestion of whole antibodies. Illustrative methods of making antibody fragments are described, for example, in Hudson et al., Nat. Med., 2003, 9:129-134, incorporated by reference in its entirety. Methods of making scFv antibodies are described, for example, in Plückthun, in The Pharmacology of Monoclonal Antibodies, vol. 113, Rosenburg and Moore eds., Springer-Verlag, New York, pp. 269-315 (1994); WO 93/16185; and U.S. Pat. Nos. 5,571,894 and 5,587,458; each of which is incorporated by reference in its entirety.

### 4.7. Methods of Making Alternative Scaffolds

The alternative scaffolds provided herein may be made by any suitable method, including the illustrative methods described herein or those known in the art. For example, methods of preparing Adnectins™ are described in Emanuel et al., mAbs, 2011, 3:38-48, incorporated by reference in its entirety. Methods of preparing iMabs are described in U.S. Pat. Pub. No. 2003/0215914, incorporated by reference in its entirety. Methods of preparing Anticalins® are described in Vogt and Skerra, Chem. Biochem., 2004, 5:191-199, incorporated by reference in its entirety. Methods of preparing Kunitz domains are described in Wagner et al., Biochem. & Biophys. Res. Comm., 1992, 186:118-1145, incorporated by reference in its entirety. Methods of preparing thioredoxin peptide aptamers are provided in Geyer and Brent, Meth. Enzymol., 2000, 328:171-208, incorporated by reference in its entirety. Methods of preparing Affibodies are provided in Fernandez, Curr. Opinion in Biotech., 2004, 15:364-373, incorporated by reference in its entirety. Methods of preparing DARPins are provided in Zahnd et al., J. Mol. Biol., 2007, 369:1015-1028, incorporated by reference in its entirety. Methods of preparing Affilins are provided in Ebersbach et al., J. Mol. Biol., 2007, 372:172-185, incorporated by reference in its entirety. Methods of preparing Tetranectins are provided in Graversen et al., J. Biol. Chem., 2000, 275:37390-37396, incorporated by reference in its entirety. Methods of preparing Avimers are provided in Silverman et al., Nature Biotech., 2005, 23:1556-1561, incorporated by reference in its entirety. Methods of preparing Fynomers are provided in Silacci et al., J. Biol. Chem., 2014, 289:14392-14398, incorporated by reference in its entirety.

Further information on alternative scaffolds is provided in Binz et al., Nat. Biotechnol., 2005 23:1257-1268; and Skerra, Current Opin. in Biotech., 2007 18:295-304, each of which is incorporated by reference in its entirety.

### 4.8. Methods of Making Multispecific Antibodies

The multispecific antibodies provided herein may be made by any suitable method, including the illustrative methods described herein or those known in the art. Methods of making common light chain antibodies are described in Merchant et al., Nature Biotechnol., 1998, 16:677-681, incorporated by reference in its entirety. Methods of making tetravalent bispecific antibodies are described in Coloma and Morrison, Nature Biotechnol., 1997, 15:159-163, incorporated by reference in its entirety. Methods of making hybrid immunoglobulins are described in Milstein and Cuello, Nature, 1983, 305:537-540; and Staerz and Bevan, Proc. Natl. Acad. Sci. USA, 1986, 83:1453-1457; each of which is incorporated by reference in its entirety. Methods of making immunoglobulins with knobs-into-holes modification are described in U.S. Pat. No. 5,731,168, incorporated by reference in its entirety. Methods of making immunoglobulins with electrostatic modifications are provided in WO 2009/089004, incorporated by reference in its entirety. Methods of making bispecific single chain antibodies are described in Traunecker et al., EMBO J., 1991, 10:3655-3659; and Gruber et al., J. Immunol., 1994, 152:5368-5374; each of which is incorporated by reference in its entirety. Methods of making single-chain antibodies, whose linker length may be varied, are described in U.S. Pat. Nos. 4,946,778 and 5,132,405, each of which is incorporated by reference in its entirety. Methods of making diabodies are described in Hollinger et al., Proc. Natl. Acad. Sci. USA, 1993, 90:6444-6448, incorporated by reference in its entirety. Methods of making triabodies and tetrabodies are described in Todorovska et al., J. Immunol. Methods, 2001, 248:47-66, incorporated by reference in its entirety. Methods of making trispecific F(ab′)3 derivatives are described in Tutt et al. J. Immunol., 1991, 147:60-69, incorporated by reference in its entirety. Methods of making cross-linked antibodies are described in U.S. Pat. No. 4,676,980; Brennan et al., Science, 1985, 229:81-83; Staerz, et al. Nature, 1985, 314:628-631; and EP 0453082; each of which is incorporated by reference in its entirety. Methods of making antigen-binding domains assembled by leucine zippers are described in Kostelny et al., J. Immunol., 1992, 148:1547-1553, incorporated by reference in its entirety. Methods of making antibodies via the DNL approach are described in U.S. Pat. Nos. 7,521,056; 7,550,143; 7,534,866; and 7,527,787; each of which is incorporated by reference in its entirety. Methods of making hybrids of antibody and non-antibody molecules are described in WO 93/08829, incorporated by reference in its entirety, for examples of such antibodies. Methods of making DAF antibodies are described in U.S. Pat. Pub. No. 2008/0069820, incorporated by reference in its entirety. Methods of making antibodies via reduction and oxidation are described in Carlring et al., PLoS One, 2011, 6:e22533, incorporated by reference in its entirety. Methods of making DVD-Igs™ are described in U.S. Pat. No. 7,612,181, incorporated by reference in its entirety. Methods of making DARTs™ are described in Moore et al., Blood, 2011, 117:454-451, incorporated by reference in its entirety. Methods of making DuoBodies® are described in Labrijn et al., Proc. Natl. Acad. Sci. USA, 2013, 110:5145-5150; Gramer et al., mAbs, 2013, 5:962-972; and Labrijn et al., Nature Protocols, 2014, 9:2450-2463; each of which is incorporated by reference in its entirety. Methods of making antibodies comprising scFvs fused to the C-terminus of the C_{H3} from an IgG are described in Coloma and Morrison, Nature Biotechnol., 1997, 15:159-163, incorporated by reference in its entirety. Methods of making antibodies in which a Fab molecule is attached to the constant region of an immunoglobulin are described in Miler et al., J. Immunol., 2003, 170:4854-4861, incorporated by reference in its entirety. Methods of making CovX-Bodies are described in Doppalapudi et al., Proc. Natl. Acad. Sci. USA, 2010, 107:22611-22616, incorporated by reference in its entirety. Methods of making Fcab antibodies are described in Wozniak-Knopp et al., Protein Eng. Des. Sel., 2010, 23:289-297, incorporated by reference in its entirety. Methods of making TandAb® antibodies are described in Kipriyanov et al., J. Mol. Biol., 1999, 293:41-56 and Zhukovsky et al., Blood, 2013, 122:5116, each of which is incorporated by reference in its entirety. Methods of making tandem Fabs are described in WO 2015/103072, incorporated by reference in its entirety. Methods of making Zybodies™ are described in LaFleur et al., mAbs, 2013, 5:208-218, incorporated by reference in its entirety.

### 4.9. Methods of Making Variants

In some embodiments, an antibody provided herein is an affinity matured variant of a parent antibody, which may be generated, for example, using phage display-based affinity maturation techniques. Briefly, one or more CDR residues may be mutated and the variant antibodies, or portions thereof, displayed on phage and screened for affinity. Such alterations may be made in CDR “hotspots,” or residues encoded by codons that undergo mutation at high frequency during the somatic maturation process (see Chowdhury, Methods Mol. Biol., 2008, 207:179-196, incorporated by reference in its entirety), and/or residues that contact the antigen.

Any suitable method can be used to introduce variability into a polynucleotide sequence(s) encoding an antibody, including error-prone PCR, chain shuffling, and oligonucleotide-directed mutagenesis such as trinucleotide-directed mutagenesis (TRIM). In some aspects, several CDR residues (e.g., 4-6 residues at a time) are randomized. CDR residues involved in antigen binding may be specifically identified, for example, using alanine scanning mutagenesis or modeling. CDR-H3 and CDR-L3 in particular are often targeted for mutation.

The introduction of diversity into the variable regions and/or CDRs can be used to produce a secondary library. The secondary library is then screened to identify antibody variants with improved affinity. Affinity maturation by constructing and reselecting from secondary libraries has been described, for example, in Hoogenboom et al., Methods in Molecular Biology, 2001, 178:1-37, incorporated by reference in its entirety.

### 4.10. Vectors, Host Cells, and Recombinant Methods

Also provided are isolated nucleic acids encoding TF antibodies, vectors comprising the nucleic acids, and host cells comprising the vectors and nucleic acids, as well as recombinant techniques for the production of the antibodies.

For recombinant production of an antibody, the nucleic acid(s) encoding it may be isolated and inserted into a replicable vector for further cloning (i.e., amplification of the DNA) or expression. In some aspects, the nucleic acid may be produced by homologous recombination, for example as described in U.S. Pat. No. 5,204,244, incorporated by reference in its entirety.

Many different vectors are known in the art. The vector components generally include one or more of the following: a signal sequence, an origin of replication, one or more marker genes, an enhancer element, a promoter, and a transcription termination sequence, for example as described in U.S. Pat. No. 5,534,615, incorporated by reference in its entirety.

Illustrative examples of suitable host cells are provided below. These host cells are not meant to be limiting, and any suitable host cell may be used to produce the antibodies provided herein.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. lichenformis), Pseudomonas (P. aeruginosa), and Streptomyces. One useful E. coli cloning host is E. coli 294, although other strains such as E. coli B, E. coli X1776, and E. coli W3110 are also suitable.

In addition to prokaryotes, eukaryotic microbes such as filamentous fungi or yeast are also suitable cloning or expression hosts for TF antibody-encoding vectors. Saccharomyces cerevisiae, or common baker's yeast, is a commonly used lower eukaryotic host microorganism. However, a number of other genera, species, and strains are available and useful, such as Schizosaccharomyces pombe, Kluyveromyces (K. lactis, K. fragilis, K. bulgaricus K. wickeramii, K. waltii, K. drosophilarum, K. thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells, baby hamster kidney (BHK) cells, Chinese hamster ovary (CHO), mouse sertoli cells, African green monkey kidney cells (VERO-76), and the like.

The host cells used to produce the TF antibody of this invention may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469; or WO 90/03430 and WO 87/00195 may be used. Each of the foregoing references is incorporated by reference in its entirety.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. For example, Carter et al. (Bio/Technology, 1992, 10:163-167, incorporated by reference in its entirety) describes a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation.

In some embodiments, the antibody is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the antibody may be useful, for example, where the antibody accumulates in a cell as an insoluble aggregate, or where yields from periplasmic expression are low.

Where the antibody is secreted into the medium, supernatants from such expression systems are generally first concentrated using a commercially available protein concentration filter, for example, an Amicon® or Millipore® Pellcon® ultrafiltration unit. A protease inhibitor such as PMSF may be included in any of the foregoing steps to inhibit proteolysis and antibiotics may be included to prevent the growth of adventitious contaminants. The antibody composition prepared from the cells can be purified using, for example, hydroxylapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the antibody. Protein A can be used to purify antibodies that comprise human γ1, γ2, or γ4 heavy chains (Lindmark et al., J. Immunol. Meth., 1983, 62:1-13, incorporated by reference in its entirety). Protein G is useful for all mouse isotypes and for human 73 (Guss et al., EMBO J., 1986, 5:1567-1575, incorporated by reference in its entirety).

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H3} domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art.

Following any preliminary purification step(s), the mixture comprising the antibody of interest and contaminants may be subjected to low pH hydrophobic interaction chromatography using an elution buffer at a pH between about 2.5 to about 4.5, generally performed at low salt concentrations (e.g., from about 0 to about 0.25 M salt).

### 5. Cytotoxic Agents

In some embodiments, ADCs provided herein comprise a cytotoxic agent. The cytotoxic agents provided herein include various anti-tumor or anti-cancer agents known in the art. In some embodiments, the cytotoxic agents cause destruction of cancer cells. In some embodiments, the cytotoxic agents inhibit the growth or proliferation of cancer cells.

Suitable cytotoxic agents include anti-angiogenic agents, pro-apoptotic agents, anti-mitotic agents, anti-kinase agents, alkylating agents, hormones, hormone agonists, hormone antagonists, chemokines, drugs, prodrugs, toxins, enzymes, antimetabolites, antibiotics, alkaloids, and radioactive isotopes.

In some embodiments, the cytotoxic agent comprises at least one of: calicheamycin, camptothecin, carboplatin, irinotecan, SN-38, carboplatin, camptothecan, cyclophosphamide, cytarabine, dacarbazine, docetaxel, dactinomycin, daunorubicin, doxorubicin, doxorubicin, etoposide, idarubicin, topotecan, vinca alkaloid, maytansinoid, maytansinoid analog, pyrrolobenzodiazepine, taxoid, duocarmycin, dolastatin, auristatin and derivatives thereof. In certain embodiments, the cytotoxic agent is monomethyl auristatin E (MMAE).

In some embodiments, the cytotoxic agent is a diagnostic agent, such as a radioactive isotope, a metal chelator, an enzyme, a fluorescent compound, a bioluminescent compound, or a chemiluminescent compound.

In some embodiments, the cytotoxic agent is a cytotoxic payload improved safety profile, for example XMT-1267 and other cytotoxic payloads described in Trail et al., Pharmacol Ther, 2018, 181:126-142.

### 6. Linkers

In some embodiments, ADCs provided herein comprise a linker. In some embodiments, an unbound linker comprises two reactive termini: an antibody conjugation reactive termini and an cytotoxic agent conjugation reactive termini. The antibody conjugation reactive terminus of the linker can be conjugated to the antibody through a cysteine thiol or lysine amine group on the antibody, typically a thiol-reactive group such as a double bond, a leaving group such as a chloro, bromo or iodo, an R-sulfanyl group or sulfonyl group, or an amine-reactive group such as a carboxyl group. The cytotoxic agent conjugation reactive terminus of the linker can be conjugated to the cytotoxic agent through formation of an amide bond with a basic amine or carboxyl group on the cytotoxin, typically a carboxyl or basic amine group.

In some embodiments, the linker is a non-cleavable linker. In some embodiments, the linker is a cleavable linker. In some embodiments, the cytotoxic agent is released from the ADC in a cell.

Suitable linkers of ADCs include labile linkers, acid labile linkers (e.g., hydrazone linkers), photolabile linkers, charged linkers, disulfide-containing linkers, peptidase-sensitive linkers (e.g., peptide linkers comprising amino acids, for example, valine and/or citrulline such as citrulline-valine or phenylalanine-lysine), β-glucuronide-linkers (See e.g., Graaf et al., Curr Pharm Des, 2002, 8:1391-1403), dimethyl linkers (See e.g., Chari et al., Cancer Research, 1992, 52:127-131; U.S. Pat. No. 5,208,020), thio-ether linkers, or hydrophilic linkers (See e.g., Kovtun et al., Cancer Res., 2010, 70:2528-2537). In certain embodiments, the cytotoxic agent is conjugated to the antibody using a valine-citrulline (vc) linker.

### 7. Methods for Making Antibody-Drug Conjugates

The antibody-drug conjugates (ADCs) provided herein can be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfoSIAB, sulfo-SMCC, and sulfo-SMPB, and SVSB (succinimidyl-(4-vinylsulfone)benzoate)). For example, a ricin immunotoxin can be prepared as described in Vitetta et al., Science, 1987, 238:1098. Additionally, the ADCs can be prepared using any suitable methods as disclosed in the art, e.g., in Bioconjugate Techniques, 2nd Ed., G. T. Hermanson, ed., Elsevier, San Francisco, 2008.

In some embodiments, the ADCs are made with site-specific conjugation techniques, resulting in homogeneous drug loading and avoiding ADC subpopulations with altered antigen-binding or pharmacokinetics. In some embodiments, “thiomabs” comprising cysteine substitutions at positions on the heavy and light chains are engineered to provide reactive thiol groups that do not disrupt immunoglobulin folding and assembly or alter antigen binding (Junutula et al., J. Immunol. Meth., 2008, 332: 41-52; Junutula et al., Nat. Biotechnol., 2008, 26: 925-932,). In some embodiments, selenocysteine is co-translationally inserted into an antibody sequence by recoding the stop codon UGA from termination to selenocysteine insertion, allowing site specific covalent conjugation at the nucleophilic selenol group of selenocysteine in the presence of the other natural amino acids (See e.g., Hofer et al., Proc. Natl. Acad. Sci. USA, 2008, 105:12451-12456; Hofer et al., Biochemistry, 2009, 48(50):12047-12057). In certain embodiments, ADCs were synthesized as described in Behrens et al., Mol Pharm, 2015, 12:3986-98.

### 8. Assays

A variety of assays known in the art may be used to identify and characterize anti-TF antibodies and anti-TF ADCs provided herein.

### 8.1. Binding, Competition, and Epitope Mapping Assays

Specific antigen-binding activity of the antibodies provided herein may be evaluated by any suitable method, including using SPR, BLI, RIA and MSD-SET, as described elsewhere in this disclosure. Additionally, antigen-binding activity may be evaluated by ELISA assays and Western blot assays.

Assays for measuring competition between two antibodies, or an antibody and another molecule (e.g., one or more ligands of TF) are described elsewhere in this disclosure and, for example, in Harlow and Lane, Antibodies: A Laboratory Manual ch. 14, 1988, Cold Spring Harbor Laboratory, Cold Spring Harbor, N.Y, incorporated by reference in its entirety.

Assays for mapping the epitopes to which the antibodies provided herein bind are described, for example, in Morris “Epitope Mapping Protocols,” in Methods in Molecular Biology vol. 66, 1996, Humana Press, Totowa, N.J., incorporated by reference in its entirety. In some embodiments, the epitope is determined by peptide competition. In some embodiments, the epitope is determined by mass spectrometry. In some embodiments, the epitope is determined by crystallography.

### 8.2. Thrombin Generation, FXa Conversion, and TF Signaling Assays

Thrombin generation in the presence of the antibodies provided herein can be determined by the Thrombin Generation Assay (TGA), as described elsewhere in this disclosure.

Assays for measuring FXa conversion in the presence of the antibodies provided herein are described elsewhere in this disclosure.

Inhibition of TF signaling can be determined by measuring the production of a cytokine regulated by the TF signaling, such as IL8 and GM-CSF. Assays for determining the IL8 and/or GM-CSF level are provided elsewhere in this disclosure and, for example, in Hjortoe et al., Blood, 2004, 103:3029-3037.

### 8.3. Assays for Effector Functions

Effector function following treatment with the antibodies provided herein may be evaluated using a variety of in vitro and in vivo assays known in the art, including those described in Ravetch and Kinet, Annu. Rev. Immunol., 1991, 9:457-492; U.S. Pat. Nos. 5,500,362, 5,821,337; Hellstrom et al., Proc. Nat'l Acad. Sci. USA, 1986, 83:7059-7063; Hellstrom et al., Proc. Nat'l Acad. Sci. USA, 1985, 82:1499-1502; Bruggemann et al., J. Exp. Med., 1987, 166:1351-1361; Clynes et al., Proc. Nat'l Acad. Sci. USA, 1998, 95:652-656; WO 2006/029879; WO 2005/100402; Gazzano-Santoro et al., J. Immunol. Methods, 1996, 202:163-171; Cragg et al., Blood, 2003, 101:1045-1052; Cragg et al. Blood, 2004, 103:2738-2743; and Petkova et al., Int'l. Immunol., 2006, 18:1759-1769; each of which is incorporated by reference in its entirety.

### 8.4. Cytotoxicity Assays and In Vivo Studies

Assays for evaluating cytotoxicity of the antibody-drug conjugates (ADCs) provided herein are described elsewhere in this disclosure.

Xenograft studies in immune compromised mice for evaluating the in vivo efficacy of the ADCs provided herein are described elsewhere in this disclosure.

Syngeneic studies in immune competent mice for evaluating the in vivo efficacy of the ADCs are included in this disclosure.

### 8.5. Immunohistochemistry (IHC) Assays

Immunohistochemistry (IHC) assays for evaluating the TF expression in patient samples are described elsewhere in this disclosure.

### 8.6. Chimeric Construct Mapping and Epitope Binning Assays

Epitope binding differences between the anti-human TF antibodies provided herein can be determined by the chimeric TF construct mapping experiments and the epitope binning assays, as described elsewhere in this disclosure.

### 9. Pharmaceutical Compositions

The antibodies or ADCs provided herein can be formulated in any appropriate pharmaceutical composition and administered by any suitable route of administration.

Suitable routes of administration include, but are not limited to, the intravitreal, intraarterial, intradermal, intramuscular, intraperitoneal, intravenous, nasal, parenteral, pulmonary, and subcutaneous routes.

The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

### 9.1. Parenteral Dosage Forms

In certain embodiments, the antibodies or ADCs provided herein are formulated as parenteral dosage forms. Parenteral dosage forms can be administered to subjects by various routes including, but not limited to, subcutaneous, intravenous (including infusions and bolus injections), intramuscular, and intraarterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry (e.g., lyophilized) products ready to be dissolved or suspended in a pharmaceutically acceptable vehicle for injection, suspensions ready for injection, and emulsions.

### 10. Dosage and Unit Dosage Forms

In human therapeutics, the doctor will determine the posology which he considers most appropriate according to a preventive or curative treatment and according to the age, weight, condition and other factors specific to the subject to be treated.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibodies or ADCs.

The amount of the antibody/ADC or composition which will be effective in the prevention or treatment of a disorder or one or more symptoms thereof can vary with the nature and severity of the disease or condition, and the route by which the antibody/ADC is administered. The frequency and dosage can also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the antibodies or ADCs provided herein are also encompassed by the dosage amounts and dose frequency schedules provided herein. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

As discussed in more detail elsewhere in this disclosure, an antibody or ADC provided herein may optionally be administered with one or more additional agents useful to prevent or treat a disease or disorder. The effective amount of such additional agents may depend on the amount of ADC present in the formulation, the type of disorder or treatment, and the other factors known in the art or described herein.

### 11. Therapeutic Applications

For therapeutic applications, the antibodies or ADCs of the invention are administered to a mammal, generally a human, in a pharmaceutically acceptable dosage form such as those known in the art and those discussed above. For example, the antibodies or ADCs of the invention may be administered to a human intravenously as a bolus or by continuous infusion over a period of time, by intravitreal, intramuscular, intraperitoneal, intra-cerebrospinal, subcutaneous, intra-articular, intrasynovial, intrathecal, or intratumoral routes. The antibodies or ADCs also are suitably administered by peritumoral, intralesional, or perilesional routes, to exert local as well as systemic therapeutic effects. The intraperitoneal route may be particularly useful, for example, in the treatment of ovarian tumors.

The antibodies or ADCs provided herein may be useful for the treatment of any disease or condition involving TF. In some embodiments, the disease or condition is a disease or condition that can benefit from treatment with an anti-TF antibody or ADC.

In some embodiments, the antibodies or ADCs provided herein are provided for use as a medicament. In some embodiments, the antibodies or ADCs provided herein are provided for use in the manufacture or preparation of a medicament. In some embodiments, the medicament is for the treatment of a disease or condition that can benefit from an anti-TF antibody or ADC.

In some embodiments, provided herein is a method of treating a disease or condition in a subject in need thereof by administering an effective amount of an anti-TF antibody or ADC provided herein to the subject.

In some embodiments, the disease or condition that can benefit from treatment with an anti-TF antibody or ADC is cancer. In some embodiments, the anti-TF antibodies or ADCs provided herein are provided for use as a medicament for the treatment of cancer. In some embodiments, the anti-TF antibodies or ADCs provided herein are provided for use in the manufacture or preparation of a medicament for the treatment of cancer. In some embodiments, provided herein is a method of treating cancer in a subject in need thereof by administering an effective amount of an anti-TF antibody or ADC provided herein to the subject.

TF is involved in thrombosis, metastasis, tumor growth, and/or tumor angiogenesis of various types of cancers, such as ovarian cancer (See Sakurai et al., Int J Gynecol Cancer, 2017, 27:37-43; Koizume et al., Biomark Cancer, 2015, 7:1-13; each of which is incorporated by reference in its entirety), cervical cancer (See Cocco et al., BMC Cancer, 2011, 11:263, incorporated by reference in its entirety), head and neck cancer (See Christensen et al., BMC Cancer, 2017, 17:572, incorporated by reference in its entirety), prostate cancer (See Yao et al., Cancer Invest., 2009, 27:430-434; Abdulkadir et al., Hum Pathol., 2009, 31:443-447; each of which is incorporated by reference in its entirety), pancreatic cancer (See Zhang et al., Oncotarget, 2017, 8:59086-59102, incorporated by reference in its entirety), triple negative breast cancer (See Zhang et al., Oncotarget, 2017, 8:59086-59102, incorporated by reference in its entirety), glioblastoma (See Guan et al., Clin Biochem., 2002, 35:321-325; Carneiro-Lobo et al., J Thromb Haemost, 2009, 7:1855-1864; each of which is incorporated by reference in its entirety), lung cancer (See Yeh et al., PLoS One, 2013, 8:e75287; Regina et al., Clin Chem., 2009, 55:1834-42; each of which is incorporated by reference in its entirety), gastric cancer (See Lo et al., Br J Cancer., 2012, 107:1125-1130, incorporated by reference in its entirety), esophageal cancer (See Chen et al., Acta Histochem., 2010, 3:233-239, incorporated by reference in its entirety), bladder cancer (See Patry et al., Int J Cancer., 2008, 122:1592-1597, incorporated by reference in its entirety), melanoma (See Bromberg et al., Proc Natl Acad Sci USA., 1995, 92:8205-8209, incorporated by reference in its entirety), and kidney cancer (See Silva et al., Int Braz J Urol., 2014, 40:499-506, incorporated by reference in its entirety).

Any suitable cancer may be treated with the antibodies or ADCs provided herein. In some embodiments, the cancer is head and neck cancer. In some embodiments, the cancer is ovarian cancer. In some embodiments, the cancer is gastric cancer. In some embodiments, the cancer is esophageal cancer. In some embodiments, the cancer is cervical cancer. In some embodiments, the cancer is prostate cancer. In some embodiments, the cancer is pancreatic cancer. In some embodiments, the cancer is estrogen receptors negative (ER−), progesterone receptors negative (PR−), and HER2 negative (HER2−) triple negative breast cancer. In some embodiments, the cancer is glioblastoma. In some embodiments, the cancer is lung cancer. In some embodiments, the cancer is bladder cancer. In some embodiments, the cancer is melanoma. In some embodiments, the cancer is kidney cancer. Additional information on the types of cancers that can be treated with anti-TF antibodies or ADCs is provided in van den Berg et al., Blood, 2012, 119:924-932, which is incorporated by reference in its entirety.

In some embodiments, provided herein is a method of delaying the onset of a cancer in a subject in need thereof by administering an effective amount of an antibody or ADC provided herein to the subject.

In some embodiments, provided herein is a method of preventing the onset of a cancer in a subject in need thereof by administering an effective amount of an antibody or ADC provided herein to the subject.

In some embodiments, provided herein is a method of reducing the size of a tumor in a subject in need thereof by administering an effective amount of an antibody or ADC provided herein to the subject.

In some embodiments, provided herein is a method of reducing the number of metastases in a subject in need thereof by administering an effective amount of an antibody or ADC provided herein to the subject.

In some embodiments, provided herein is a method for extending the period of overall survival, median survival time, or progression-free survival in a subject in need thereof by administering an effective amount of an antibody or ADC provided herein to the subject.

In some embodiments, provided herein is a method for treating a subject who has become resistant to a standard of care therapeutic by administering an effective amount of an antibody or ADC provided herein to the subject.

In some embodiments, the disease or condition that can benefit from treatment with an anti-TF antibody is a disease or condition involving neovascularization. In certain embodiments, the disease or condition involving neovascularization is age-related macular degeneration (AMD). In certain embodiments, the disease or condition involving neovascularization is diabetic retinopathy. In certain embodiments, the disease or condition involving neovascularization is cancer. In some embodiments, the disease or condition that can benefit from treatment with an anti-TF antibody is a disease or condition involving vascular inflammation.

In some embodiments, the anti-TF antibodies provided herein are provided for use as a medicament for the treatment of a disease or condition involving neovascularization. In some embodiments, the anti-TF antibodies provided herein are provided for use in the manufacture or preparation of a medicament for the treatment of a disease or condition involving neovascularization. In certain embodiments, the disease or condition involving neovascularization is age-related macular degeneration (AMD). In certain embodiments, the disease or condition involving neovascularization is diabetic retinopathy. In certain embodiments, the disease or condition involving neovascularization is cancer. In some embodiments, the anti-TF antibodies provided herein are provided for use as a medicament for the treatment of a disease or condition involving vascular inflammation. In some embodiments, the anti-TF antibodies provided herein are provided for use in the manufacture or preparation of a medicament for the treatment of a disease or condition involving vascular inflammation.

In some embodiments, provided herein is a method of treating a disease or condition involving neovascularization in a subject in need thereof by administering an effective amount of an anti-TF antibody provided herein to the subject. In certain embodiments, the disease or condition involving neovascularization is age-related macular degeneration (AMD). In certain embodiments, the disease or condition involving neovascularization is diabetic retinopathy. In certain embodiments, the disease or condition involving neovascularization is cancer. In some embodiments, provided herein is a method of treating a disease or condition involving vascular inflammation in a subject in need thereof by administering an effective amount of an anti-TF antibody provided herein to the subject.

In some embodiments, provided herein is a method of delaying the onset of a disease or condition involving neovascularization in a subject in need thereof by administering an effective amount of an antibody provided herein to the subject.

In some embodiments, provided herein is a method of preventing the onset of a disease or condition involving neovascularization in a subject in need thereof by administering an effective amount of an antibody provided herein to the subject.

In some embodiments, provided herein is a method of delaying the onset of age-related macular degeneration (AMD) in a subject in need thereof by administering an effective amount of an antibody provided herein to the subject.

In some embodiments, provided herein is a method of preventing the onset of age-related macular degeneration (AMD) in a subject in need thereof by administering an effective amount of an antibody provided herein to the subject.

In some embodiments, provided herein is a method of delaying the onset of diabetic retinopathy in a subject in need thereof by administering an effective amount of an antibody provided herein to the subject.

In some embodiments, provided herein is a method of preventing the onset of diabetic retinopathy in a subject in need thereof by administering an effective amount of an antibody provided herein to the subject.

In some embodiments, provided herein is a method of delaying the onset of a disease or condition involving vascular inflammation in a subject in need thereof by administering an effective amount of an antibody provided herein to the subject.

In some embodiments, provided herein is a method of preventing the onset of a disease or condition involving vascular inflammation in a subject in need thereof by administering an effective amount of an antibody provided herein to the subject.

### 12. Combination Therapies

In some embodiments, an antibody or ADC provided herein is administered with at least one additional therapeutic agent. Any suitable additional therapeutic agent may be administered with an antibody or ADC provided herein. In some aspects, the additional therapeutic agent is selected from radiation, a cytotoxic agent, a chemotherapeutic agent, a cytostatic agent, an anti-hormonal agent, an immunostimulatory agent, an anti-angiogenic agent, and combinations thereof.

The additional therapeutic agent may be administered by any suitable means. In some embodiments, an antibody or ADC provided herein and the additional therapeutic agent are included in the same pharmaceutical composition. In some embodiments, an antibody or ADC provided herein and the additional therapeutic agent are included in different pharmaceutical compositions.

In embodiments where an antibody or ADC provided herein and the additional therapeutic agent are included in different pharmaceutical compositions, administration of the antibody or ADC can occur prior to, simultaneously, and/or following, administration of the additional therapeutic agent.

### 13. Diagnostic Methods

Also provided are methods for detecting the presence of TF on cells from a subject. Such methods may be used, for example, to predict and evaluate responsiveness to treatment with an antibody or ADC provided herein.

In some embodiments, the method can be used to detect TF in a subject having or suspected of having a disease or condition. In some embodiments, the methods comprise (a) receiving a sample from the subject; and (b) detecting the presence or the level of TF in the sample by contacting the sample with the antibody provided herein. In some embodiments, the methods comprise (a) administering to the subject the antibody provided herein; and (b) detecting the presence or the level of TF in the subject. In some embodiments, the disease or condition is a cancer. In some embodiments, the cancer is head and neck cancer. In some embodiments, the cancer is ovarian cancer. In some embodiments, the cancer is gastric cancer. In some embodiments, the cancer is esophageal cancer. In some embodiments, the cancer is cervical cancer. In some embodiments, the cancer is prostate cancer. In some embodiments, the cancer is pancreatic cancer. In some embodiments, the cancer is estrogen receptors negative (ER−), progesterone receptors negative (PR−), and HER2 negative (HER2−) triple negative breast cancer. In some embodiments, the cancer is glioblastoma. In some embodiments, the cancer is lung cancer. In some embodiments, the cancer is bladder cancer. In some embodiments, the cancer is melanoma. In some embodiments, the cancer is kidney cancer. In some embodiments, the disease or condition involves neovascularization. In certain embodiments, the disease or condition involving neovascularization is age-related macular degeneration (AMD). In certain embodiments, the disease or condition involving neovascularization is diabetic retinopathy. In certain embodiments, the disease or condition involving neovascularization is cancer. In some embodiments, the disease or condition involves vascular inflammation.

In some embodiments, the methods comprise (a) administering to the subject the ADC provided herein; and (b) detecting the presence or the level of TF in the subject. In some embodiments, the disease or condition is a cancer. In some embodiments, the cancer is head and neck cancer. In some embodiments, the cancer is ovarian cancer. In some embodiments, the cancer is gastric cancer. In some embodiments, the cancer is esophageal cancer. In some embodiments, the cancer is cervical cancer. In some embodiments, the cancer is prostate cancer. In some embodiments, the cancer is pancreatic cancer. In some embodiments, the cancer is estrogen receptors negative (ER−), progesterone receptors negative (PR−), and HER2 negative (HER2−) triple negative breast cancer. In some embodiments, the cancer is glioblastoma. In some embodiments, the cancer is lung cancer. In some embodiments, the cancer is bladder cancer. In some embodiments, the cancer is melanoma. In some embodiments, the cancer is kidney cancer.

In some embodiments, the antibody provided herein is conjugated with a fluorescent label. In some embodiments, the antibody provided herein is conjugated with a radioactive label. In some embodiments, the antibody provided herein is conjugated with an enzyme label.

In some embodiments, the ADC provided herein comprises a fluorescent label. In some embodiments, the ADC provided herein comprises a radioactive label. In some embodiments, the ADC provided herein comprises an enzyme label.

In some embodiments, the relative amount of TF expressed by such cells is determined. The fraction of cells expressing TF and the relative amount of TF expressed by such cells can be determined by any suitable method. In some embodiments, flow cytometry is used to make such measurements. In some embodiments, fluorescence assisted cell sorting (FACS) is used to make such measurement.

### 14. Kits

Also provided are kits comprising the antibodies or ADCs provided herein. The kits may be used for the treatment, prevention, and/or diagnosis of a disease or disorder, as described herein.

In some embodiments, the kit comprises a container and a label or package insert on or associated with the container. Suitable containers include, for example, bottles, vials, syringes, and IV solution bags. The containers may be formed from a variety of materials, such as glass or plastic. The container holds a composition that is by itself, or when combined with another composition, effective for treating, preventing and/or diagnosing a disease or disorder. The container may have a sterile access port. For example, if the container is an intravenous solution bag or a vial, it may have a port that can be pierced by a needle. At least one active agent in the composition is an antibody or ADC provided herein. The label or package insert indicates that the composition is used for treating the selected condition.

In some embodiments, the kit comprises (a) a first container with a first composition contained therein, wherein the first composition comprises an antibody or ADC provided herein; and (b) a second container with a second composition contained therein, wherein the second composition comprises a further therapeutic agent. The kit in this embodiment of the invention may further comprise a package insert indicating that the compositions can be used to treat a particular condition.

Alternatively, or additionally, the kit may further comprise a second (or third) container comprising a pharmaceutically-acceptable excipient. In some aspects, the excipient is a buffer. The kit may further include other materials desirable from a commercial and user standpoint, including filters, needles, and syringes.

### EXAMPLES

The following are examples of methods and compositions of the invention. It is understood that various other embodiments may be practiced, given the general description provided herein.

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B(1992).

### Example 1: Generation of TF Antibodies

Human, cynomolgus monkey, and mouse TF extracellular domain (ECD) fragments were expressed as C-terminal His or Fcγ fragment fusions. Expi293 cells (ThermoFisher Scientific, Waltham, MA, USA) were transiently transfected as recommended by the manufacturer with pcDNA3.1V5-HisA (ThermoFisher Scientific) encoding human, cynomolgus, or mouse TF ECD-His6 (HHHHHH (SEQ ID NO:922)) (TF-His; SEQ ID NOs:811, 815, and 819, respectively) or pFUSE-hIgG1-Fc (Invivogen, San Diego, CA, USA) encoding human, cynomolgus or mouse TF ECD-Fc (TF-Fc; SEQ ID NOs:812, 816, and 820, respectively). For the His-tagged proteins, cell culture supernatants cleared from cells by centrifugation were preconditioned with 330 mM sodium chloride and 13.3 mM imidazole. Using recommended procedures, the TF-His6 and TF-Fc proteins were purified by affinity chromatography with a HisTrap HP and MabSelect SuRe column (GE Healthcare Bio-Sciences, Marlborough, MA, USA), respectively. FVII-Fc expressed in Expi293 was purified by affinity chromatography with a MabSelect SuRe column, followed by size exclusion chromatography. The TF-His6 and TF-Fc proteins were biotinylated with a 15× molar excess of Sulfo-NHS-SS-biotin as recommended (ThermoFisher Scientific). The non-labeled and biotinylated proteins were further purified by size exclusion chromatography using a Superdex 200 Increase 10/300 column (GE Healthcare Bio-Sciences).

Human antibodies against human TF were generated by Adimab™ yeast-based antibody presentation using the biotinylated recombinant TF proteins as screening antigens, as described below. All antibodies against human TF were evaluated for cross-reactivity with cynomolgus monkey and mouse TF. The binding activity of the antibodies to human, cynomolgus monkey, and mouse TF is shown in Table 5.

### I. Library Interrogation and Selection Methodology for Isolation of Anti-TF Antibodies

### Naive Library Selections

Eight naïve human synthetic yeast libraries each of ˜10⁹ diversity were designed, generated, and propagated as described previously (see, e.g., WO2009036379; WO2010105256; WO2012009568; Xu et al., Protein Eng Des Sel., 2013, 26(10):663-70). Eight parallel selections were performed, using the eight naïve libraries for monomeric human TF selections.

For the first two rounds of selection, a magnetic bead sorting technique utilizing the Miltenyi MACS system was performed, essentially as described (Siegel et al., J Immunol Methods, 2004, 286(1-2):141-53). Briefly, yeast cells (˜10¹⁰ cells/library) were incubated with 10 nM of biotinylated human TF Fc-fusion antigen for 15 min at room temperature in FACS wash buffer PBS with 0.1% BSA. After washing once with 50 mL ice-cold wash buffer, the cell pellet was resuspended in 40 mL wash buffer, and 500 μl Streptavidin MicroBeads (Miltenyi Biotec, Bergisch Gladbach, Germany; Cat #130-048-101) were added to the yeast and incubated for 15 min at 4° C. Next, the yeast were pelleted, resuspended in 5 mL wash buffer, and loaded onto a MACS LS column (Miltenyi Biotec, Bergisch Gladbach, Germany; Cat. #130-042-401). After the 5 mL was loaded, the column was washed 3 times with 3 mL FACS wash buffer. The column was then removed from the magnetic field, and the yeast were eluted with 5 mL of growth media and then grown overnight.

Subsequent to the two rounds of MACS, the following four rounds of sorting were performed using flow cytometry (FACS). For the first round of FACS, approximately 5×107 yeast were pelleted, washed three times with wash buffer, and incubated with 10 nM of each the biotinylated Fc-fusion proteins of mouse and/or cynomolgus TF antigen for 10-15 min at room temperature. Yeast were then washed twice and stained with LC-FITC diluted 1:100 (Southern Biotech, Birmingham, Alabama; Cat #2062-02) and either SA-633 (Life Technologies, Grand Island, NY; Cat #S21375) diluted 1:500, or EA-PE (Sigma-Aldrich, St Louis; Cat #E4011) diluted 1:50, secondary reagents for 15 min at 4° C. After washing twice with ice-cold wash buffer, the cell pellets were resuspended in 0.4 mL wash buffer and transferred to strainer-capped sort tubes. Sorting was performed using a FACS ARIA sorter (BD Biosciences), and sort gates were determined to select for TF binding. The mouse- and cyno-selected populations from the first round of FACS were grown out and expanded through sub-culturing in selective media. The second, third, and fourth rounds of FACS involved positive sorts to enrich for TF binders and/or negative sorts to decrease the number of non-specific binders using soluble membrane proteins from CHO cells (see, e.g., WO2014179363 and Xu et al., PEDS, 2013, 26(10):663-70). After the final round of sorting, yeast were plated and sequenced.

### Affinity Maturation of Clones Identified in Naïve Selections

Heavy chains from the naïve outputs (described above) were used to prepare light chain diversification libraries, which were then used for additional selection rounds. In particular, heavy chain variable regions were extracted from the fourth naïve selection round outputs and transformed into a light chain library with a diversity of 1×10⁶.

The first of selection round utilized Miltenyi MACS beads and 10 nM biotinylated human TF Fc-fusion as antigen. Subsequent to the MACS bead selections, three rounds of FACS sorting were performed as described above using cynomolgus and mouse Fc-fusion TF at 10 nM or either biotinylated Fc-fusion TF antigens or biotinylated monomeric HIS-forms of human, mouse or cynomolgus TF. Individual colonies from each FACS selection round were sequenced.

Optimization of Leads Identified from Naïve or Light Chain Diversification Selections

Optimization of lead clones was carried out utilizing three maturation strategies: diversification of CDR-H1 and CDR-H2; diversification of CDR-H3 following CDR-H1 and CDR-H2 diversity pool optimization; and diversification of CDR-L3 within selected CDR-L1 and CDR-L2 diversity pools.

CDR-H1 and CDR-H2 selection: The CDR-H3s from clones selected from either naïve or light chain diversification procedure were recombined into a premade library with CDR-H1 and CDR-H2 variants of a diversity of 1×10⁸ and selections were performed using biotinylated Fc-fusion cynomolgus TF antigen, biotinylated cynomolgus HIS-TF antigen, and/or biotinylated human HIS-TF. Affinity pressures were applied by using decreasing concentrations of biotinylated HIS-TF antigens (down to 1 nM) under equilibrium conditions at room temperature.

CDR-H3/CDR-H1/CDR-H2 selections: Oligos were ordered from IDT which comprised the CDR-H3 as well as a homologous flanking region on either side of the CDR-H3. Amino acid positions in the CDR-H3 were variegated via NNK diversity at two positions per oligo across the entire CDR-H3. The CDR-H3 oligos were double-stranded using primers which annealed to the flanking region of the CDR-H3. The remaining FR1 to FR3 of the heavy chain variable region was amplified from pools of antibodies with improved affinity that were isolated from the CDR-H1 and CDR-H2 diversities selected above. The library was then created by transforming the double stranded CDR-H3 oligo, the FR1 to FR3 pooled fragments, and the heavy chain expression vector into yeast already containing the light chain of the parent. Selections were performed as during previous cycles using FACS sorting. FACS rounds assessed non-specific binding, species cross-reactivity, and affinity pressure, and sorting was performed to obtain populations with the desired characteristics. Affinity pressures for these selections were performed as described above in the CDR-H1 and CDR-H2 selection.

CDR-L3/CDR-L1/CDR-L2 selections: Oligos were ordered from IDT which comprised the CDR-L3 as well as a homologous flanking region on either side of the CDR-L3. Amino acid positions in the CDR-L3 were variegated via NNK diversity at one position per oligo across the entire CDR-L3. The CDR-L3 oligos were double-stranded using primers which annealed to the flanking region of the CDR-L3. The remaining FR1 to FR3 of the light chain variable region was amplified from pools of antibodies with improved affinity that were isolated from the CDR-L1 and CDR-L2 diversities selected above. The library was then created by transforming the double stranded CDR-L3 oligo, the FR1 to FR3 pooled fragments, and the light chain expression vector into yeast already containing the heavy chain of the parent. Selections were performed as during previous cycles using FACS sorting. FACS rounds assessed non-specific binding, species cross-reactivity, and affinity pressure, and sorting was performed to obtain populations with the desired characteristics. Affinity pressures included titrations as well as incorporation of the parental Fab in antigen pre-complexation.

### II. IgG and Fab Production and Purification

In order to produce sufficient amounts of selected antibodies for further characterization, the yeast clones were grown to saturation and then induced for 48 h at 30° C. with shaking. After induction, yeast cells were pelleted and the supernatants were harvested for purification. IgGs were purified using a Protein A column and eluted with acetic acid, pH 2.0. Fab fragments were generated by papain digestion and purified over CaptureSelect IgG-CH1 affinity matrix (LifeTechnologies, Cat #1943200250).

### Example 2: Binding Affinity Assay

Kinetic measurements for the anti-TF antibodies were conducted on an Octet QK384 (Pall ForteBio, Fremont, CA, USA) or a Biacore (GE Healthcare Bio-Sciences).

ForteBio affinity measurements were performed generally as previously described (Estep et al., MAbs. 2013 March-April; 5(2):270-8). Briefly, ForteBio affinity measurements were performed by loading IgGs on-line onto AHC sensors. Sensors were equilibrated off-line in assay buffer for 30 min and then monitored on-line for 60 seconds for baseline establishment. Sensors with loaded IgGs were exposed to 100 nM antigen (human, cynomolgus, or mouse TF) for 3 min, afterwards they were transferred to assay buffer for 3 min for off-rate measurement. Alternatively, binding measurements were obtained by loading biotinylated TF monomer on SA sensors followed by exposure to 100 nM antibody Fab in solution. Kinetic data was analyzed and fitted using a 1:1 Langmuir binding model and the K_{D} was calculated by dividing the k_{off} by the kₒₙ. The K_{D} values of the TF antibodies measured by the Octet-based experiments are shown in Table 5.

For the Biacore-based measurements, the antibody was covalently coupled to a CM5 or C1 chip using an amine-coupling kit (GE Healthcare Bio-Sciences). Association between the anti-TF antibodies and a five-point three-fold titration of TF-His starting at 25 to 500 nM was measured for 300 sec. Subsequently, dissociation between the anti-TF antibody and TF-His was measured for up to 1800 sec. Kinetic data was analyzed and fitted globally using a 1:1 binding model. The K_{D} values of the TF antibodies measured by the Biacore-based experiments are shown in Table 5.

As shown in Table 5, the affinity of the antibodies for hTF, as indicated by K_{D}, is between 10-7 M and 10⁻¹¹ M. All anti-hTF antibodies are cross-reactive with cTF. In addition, all anti-hTF antibodies from groups 25 and 43 exhibit binding activity to mTF. The anti-hTF antibodies 25G, 25G1, 25G9, and 43D8 are cross-reactive with mTF. There are no other known human or humanized anti-hTF monoclonal antibodies that exhibit binding activity and cross-reactivity to mouse TF, indicating that the antibodies from groups 25 and 43 bind to a novel TF epitope.

TABLE 5
Antibody Kinetics

| | Human K_{D} | Cynomolgus | Mouse K_{D} | Human K_{D} | Cynomolgus | Mouse K_{D} |
|---|---|---|---|---|---|---|
| | (nM) | K_{D} (nM) | (nM) | (nM) | K_{D} (nM) | (nM) |
| Ab | [Biacore] | [Biacore] | [Biacore] | [ForteBio] | [ForteBio] | [ForteBio] |
| 1F | 0.31 | 0.26 | nd* | 1.28 | 1.43 | no binding* |
| 1G | nd* | nd* | nd* | 2.20 | 2.70 | nd* |
| 25A | 6.20 | 5.42 | nd* | 8.45 | 7.65 | 263 |
| 25A3 | 0.36 | 0.21 | nd* | 1.67 | 1.36 | 131 |
| 25A5 | 0.08 | 0.04 | nd* | 0.64 | 0.76 | 188 |
| 25G | 23.0 | 18.0 | nd* | 21.9 | 17.5 | 114 |
| 25G1 | 0.94 | 0.78 | 5.4 | 3.97 | 4.99 | 34.2 |
| 25G9 | 13.3 | 16.4 | 2.9 | 35.8 | 42.9 | 9.16 |
| 29D | nd* | nd* | nd* | 3.30 | 12.0 | nd |
| 29E | 0.47 | 5.06 | nd* | 2.32 | 15.0 | no binding* |
| 39A | 0.09 | 0.08 | nd* | 0.83 | 0.57 | no binding* |
| 43B | 1.75 | 5.64 | nd* | 2.40 | 3.40 | 161 |
| 43B1 | 0.07 | 0.12 | nd* | 0.96 | 1.05 | 72.1 |
| 43B7 | 0.14 | 0.24 | nd* | 0.86 | 0.94 | 360 |
| 43D | 2.09 | 5.66 | nd* | 3.84 | 4.12 | 281 |
| 43D7 | 0.06 | 0.12 | 21 | 1.02 | 1.11 | 41.4 |
| 43D8 | 0.15 | 0.39 | 2.4 | 1.61 | 1.96 | 6.12 |
| 43E | 1.46 | 5.69 | nd* | 2.52 | 4.07 | 121 |
| 43Ea | 1.60 | 6.42 | nd* | 2.28 | 2.71 | 140 |
| 54E | 0.42 | 1.83 | nd* | 1.59 | 4.16 | no binding* |

no binding*: no to weak binding, with no reportable K_{D}
nd*: not determined

### Example 3: Cell-Based Binding Assay

HCT116 cells with endogenous expression of human TF were obtained from the American Tissue Culture Collection (ATCC, Manassas, VA, USA) and were maintained as recommended. Flp-In-CHO cells expressing mouse TF were generated by transfection of Flp-In-CHO cells as recommended with a pcDNA5/FRT vector (ThermoFisher Scientific) encoding full-length mouse TF with a C-terminal FLAG tag. A mouse TF-positive CHO clone was isolated by limiting dilution in tissue culture-treated 96-well plates.

Cell-based antibody binding was assessed as previously described in Liao-Chan et al., PLoS One, 2015, 10:e0124708, which is incorporated by reference in its entirety. 1.2×10⁵ cells collected with Cellstripper (Mediatech, Manassas, VA, USA) were incubated with a twelve-point 1:3 dilution titration of anti-human TF IgG1 or Fab antibody starting at 250 nM or 100 nM for 2 hr on ice. After 2 washes, cells labeled with IgG1 or Fab were incubated for min on ice with 150 nM of Goat Phycoerythrin (PE) F(ab′)₂ fragment goat anti-human IgG, Fcγ fragment specific (Jackson ImmunoResearch, West Grove, PA, USA) or FITC-labeled F(ab′)₂ fragment goat anti-human kappa (SouthernBiotech, Birmingham, AL, USA), respectively. After 2 washes, dead cells were labeled with TO-PRO-3 Iodide (ThermoFisher Scientific) and samples were analyzed on a CytoFLEX flow cytometer (Beckman Coulter, Brea, CA, USA) or Novocyte flow cytometer (ACEA Biosciences, San Diego, CA, USA). The median fluorescence intensities (MFIs) at each dilution were plotted and cell EC₅₀'s were derived using a 4-parameter binding model in Prism (GraphPad, La Jolla, CA, USA). The results of binding of anti-TF antibodies to human TF-positive HCT-116 cells are shown in FIGS. 1A and 1B. The results of binding of anti-TF antibodies to CHO cells expressing mouse TF are shown in FIGS. 2A and 2B.

All anti-hTF antibodies in FIGS. 1A and 1B exhibit high affinity to human TF-positive HCT-116 cells with an EC₅₀ ranging from about 687 pM to about 39 pM. Antibodies from groups 25 and 43 exhibit binding to CHO cells expressing mouse TF with an EC₅₀ ranging from about 455 nM to about 2.9 nM, as shown in FIGS. 2A and 2B. The binding activity to mouse TF is a unique property of the anti-hTF antibodies from groups 25 and 43. This is advantageous for pre-clinical studies of these antibodies with mouse models.

### Example 4: Thrombin Generation Assay (TGA)

The TGA assay was performed using the calibrated-automated-thrombogram (CAT) instrument manufactured and distributed by STAGO. The test method design was equivalent to a standard CAT assay measurement, except that the plasma source was NPP in citrate/CTI. The anti-TF antibodies were titrated at 0, 10, 50 and 100 nM and mixed with normal pooled plasma (NPP) collected in 11 mM citrate supplemented with 100 microgram/mL of corn trypsin inhibitor (citrate/CTI). Relipidated TF was added to a 96-well assay plate, followed by addition of the antibody/NPP mixture. After a 10-min incubation or directly after combining the relipidated TF with antibody/NPP, thrombin generation was initiated by the addition of calcium and the thrombin substrate. The STAGO software was used to report the following parameters: Peak IIa (highest thrombin concentration generated [nM]); Lag Time (time to IIa generation [min]); ETP (endogenous thrombin potential, area under the curve [nM×min]); and ttPeak (time to Peak IIa [min]). Percent peak thrombin generation (% Peak IIa) and percent endogenous thrombin potential (% ETP) in the presence of each antibody relative to a no antibody plasma control on the same plate were also reported.

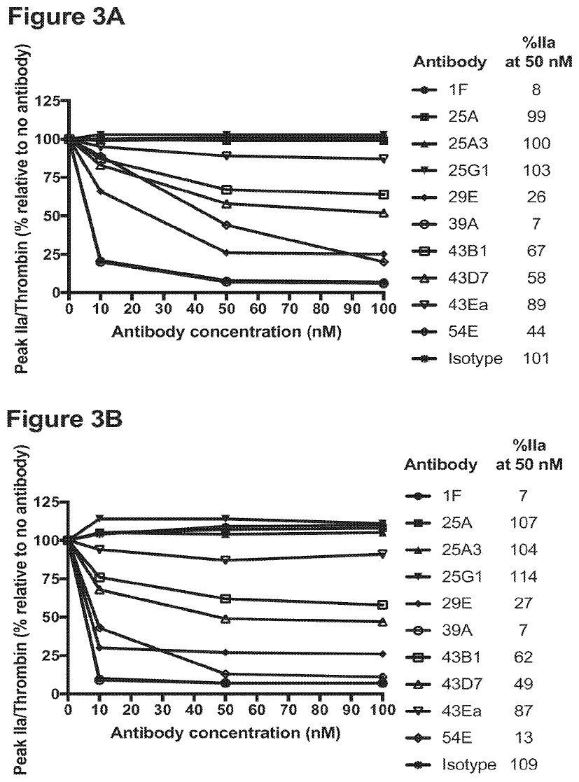

The Peak IIa, Lag Time, ETP, ttPeak, % Peak IIa, and % ETP in the presence of each antibody selected from 1F, 25A, 25A3, 25G1, 29E, 39A, 43B1, 43D7, 43Ea, and 54E without antibody incubation prior to addition of calcium and thrombin substrate are shown in Table 6. The Peak IIa, Lag Time, ETP, ttPeak, % Peak IIa, and % ETP in the presence of each antibody selected from 1F, 25A, 25A3, 25G1, 29E, 39A, 43B1, 43D7, 43Ea, and 54E with 10 min antibody incubation prior to addition of calcium and thrombin substrate are shown in Table 7. The % Peak IIa in the presence of titrations of anti-TF antibodies without antibody incubation prior to addition of calcium and thrombin substrate is plotted in FIG. 3A. The % Peak IIa in the presence of titrations of anti-TF antibodies with 10 min antibody incubation prior to addition of calcium and thrombin substrate is plotted in FIG. 3B.

The % Peak IIa is greater than 900 in the presence of antibodies from group 25, including 25A, 25A3, and 25G1. The 7 ETP is greater than 1000 in the presence of antibodies from group 25, including 25A, 25A3, and 25G1. The 00 Peak IIa is greater than 40% in the presence of antibodies from group 43, including 43B1, 43D7, and 43Ea. The 00 ETP is greater than 90% in the presence of antibodies from group 43, including 43B1, 43D7, and 43Ea.

This data indicates that antibodies from groups 25 and 43 allow normal thrombin generation, and therefore are not inhibitors of thrombin generation.

TABLE 6
Thrombin Generation Assay without Antibody Pre-Incubation

| | | Ab | Peak | Lag | | | % | |
|---|---|---|---|---|---|---|---|---|
| | | conc. | IIa | Time | ETP | ttPeak | Peak | % |
| Plate | Antibody | (nM) | (nM) | (mm) | (nM · min) | (mm) | IIa | ETP |
| 1 | 1F | 100 | 29 | 25.9 | * | 37.9 | 7 | * |
| | | 50 | 32 | 27.2 | * | 36.8 | 8 | * |
| | | 10 | 83 | 12.1 | 1395 | 19.8 | 21 | 58 |
| 1 | 25A | 100 | 398 | 4.4 | 2610 | 7.1 | 99 | 108 |
| | | 50 | 399 | 4.2 | 2621 | 7.1 | 99 | 108 |
| | | 10 | 403 | 4.1 | 2555 | 6.8 | 100 | 106 |
| 1 | 25A3 | 100 | 405 | 3.9 | 2493 | 6.5 | 100 | 103 |
| | | 50 | 404 | 3.9 | 2495 | 6.6 | 100 | 103 |
| | | 10 | 401 | 4.2 | 2550 | 7.3 | 99 | 106 |
| 1 | 25G1 | 100 | 416 | 4.5 | 2626 | 7.1 | 103 | 109 |
| | | 50 | 416 | 4.5 | 2680 | 7.1 | 103 | 111 |
| | | 10 | 417 | 4.5 | 2635 | 7.0 | 103 | 109 |
| 1 | 29E | 100 | 99 | 17.3 | * | 26.4 | 25 | * |
| | | 50 | 107 | 14.4 | 1747 | 22.7 | 26 | 72 |
| | | 10 | 266 | 5.7 | 2189 | 10.0 | 66 | 91 |
| 1 | 39A | 100 | 26 | 28.9 | * | 40.1 | 6 | * |
| | | 50 | 30 | 30.5 | * | 40.0 | 7 | * |
| | | 10 | 82 | 12.1 | 1330 | 19.6 | 20 | 55 |
| 1 | Plasma | NA | 403 | 4.1 | 2417 | 6.8 | 100 | 100 |
| | ctrl. | | | | | | | |
| 2 | 43B1 | 100 | 221 | 5.2 | 2167 | 10.6 | 64 | 100 |
| | | 50 | 232 | 5.2 | 2195 | 10.3 | 67 | 101 |
| | | 10 | 299 | 4.9 | 2288 | 8.9 | 87 | 105 |
| 2 | 43D7 | 100 | 179 | 5.4 | 2094 | 11.8 | 52 | 96 |
| | | 50 | 202 | 5.3 | 2116 | 11.1 | 58 | 97 |
| | | 10 | 287 | 5.0 | 2263 | 9.0 | 83 | 104 |
| 2 | 43Ea | 100 | 300 | 4.6 | 2219 | 8.1 | 87 | 102 |
| | | 50 | 307 | 4.6 | 2234 | 8.1 | 89 | 103 |
| | | 10 | 328 | 5.0 | 2329 | 8.3 | 95 | 107 |
| 2 | 54E | 100 | 68 | 14.8 | 1175 | 23.9 | 20 | 54 |
| | | 50 | 154 | 8.9 | 2019 | 15.9 | 44 | 93 |
| | | 10 | 307 | 5.7 | 2307 | 9.6 | 89 | 106 |
| 2 | Isotype | 100 | 348 | 5.0 | 2415 | 8.3 | 101 | 111 |
| | | 50 | 347 | 5.0 | 2360 | 8.0 | 101 | 109 |
| | | 10 | 346 | 4.3 | 2260 | 7.6 | 100 | 104 |
| 2 | Plasma | NA | 345 | 4.7 | 2171 | 7.8 | 100 | 100 |
| | ctrl. | | | | | | | |

* Groups with “No Tail Found” Errors when the software cannot calculate the ETP.

TABLE 7
Thrombin Generation Assay with 10 min Antibody Pre-Incubation

| | | Ab | Peak | Lag | | | % | |
|---|---|---|---|---|---|---|---|---|
| | | conc. | IIa | Time | ETP | ttPeak | Peak | % |
| Plate | Antibody | (nM) | (nM) | (mm) | (nM · min) | (mm) | IIa | ETP |
| 1 | 1F | 100 | 17 | 30.3 | * | 42.0 | 7 | * |
| | | 50 | 20 | 27.6 | * | 38.9 | 7 | * |
| | | 10 | 27 | 18.8 | 540 | 28.6 | 10 | 31 |
| 1 | 25A | 100 | 285 | 3.3 | 1898 | 6.7 | 108 | 110 |
| | | 50 | 284 | 3.3 | 1887 | 6.6 | 107 | 110 |
| | | 10 | 277 | 3.3 | 1842 | 6.7 | 105 | 107 |
| 1 | 25A3 | 100 | 277 | 3.1 | 1785 | 6.3 | 105 | 104 |
| | | 50 | 275 | 3.2 | 1824 | 6.4 | 104 | 106 |
| | | 10 | 278 | 3.2 | 1827 | 6.6 | 105 | 106 |
| 1 | 25G1 | 100 | 293 | 3.3 | 1827 | 6.4 | 111 | 106 |
| | | 50 | 301 | 3.3 | 1853 | 6.3 | 114 | 108 |
| | | 10 | 302 | 3.3 | 1891 | 6.3 | 114 | 110 |
| 1 | 29E | 100 | 68 | 15.1 | 1098 | 25.3 | 26 | 64 |
| | | 50 | 70 | 14.2 | 1168 | 24.3 | 27 | 68 |
| | | 10 | 78 | 10.4 | 1254 | 20.2 | 30 | 73 |
| 1 | 39A | 100 | 17 | 28.0 | * | 40.2 | 7 | * |
| | | 50 | 17 | 28.4 | 346 | 38.9 | 7 | 20 |
| | | 10 | 25 | 20.8 | 482 | 30.7 | 9 | 28 |
| 1 | Plasma | NA | 264 | 3.3 | 1720 | 6.8 | 100 | 100 |
| | ctrl. | | | | | | | |
| 2 | 43B1 | 100 | 152 | 3.2 | 1712 | 9.3 | 58 | 98 |
| | | 50 | 163 | 3.2 | 1797 | 9.0 | 62 | 103 |
| | | 10 | 200 | 3.2 | 1788 | 8.1 | 76 | 103 |
| 2 | 43D7 | 100 | 124 | 3.6 | 1656 | 10.3 | 47 | 95 |
| | | 50 | 128 | 3.6 | 1677 | 10.3 | 49 | 96 |
| | | 10 | 178 | 3.6 | 1745 | 8.8 | 68 | 100 |
| 2 | 43Ea | 100 | 239 | 2.9 | 1820 | 6.9 | 91 | 104 |
| | | 50 | 227 | 2.9 | 1791 | 7.1 | 87 | 103 |
| | | 10 | 247 | 3.2 | 1825 | 7.0 | 94 | 105 |
| 2 | 54E | 100 | 29 | 22.1 | 580 | 32.3 | 11 | 33 |
| | | 50 | 35 | 18.3 | 680 | 28.4 | 13 | 39 |
| | | 10 | 112 | 6.1 | 1530 | 13.4 | 43 | 88 |
| 2 | Isotype | 100 | 288 | 3.2 | 1888 | 6.6 | 110 | 108 |
| | | 50 | 285 | 3.2 | 1879 | 6.6 | 109 | 108 |
| | | 10 | 273 | 3.2 | 1804 | 6.6 | 104 | 104 |
| 2 | Plasma | NA | 262 | 3.2 | 1742 | 6.9 | 100 | 100 |
| | ctrl. | | | | | | | |

* Groups with “No Tail Found” Errors when the software cannot calculate the ETP.

### Example 5: FXa Conversion Assay

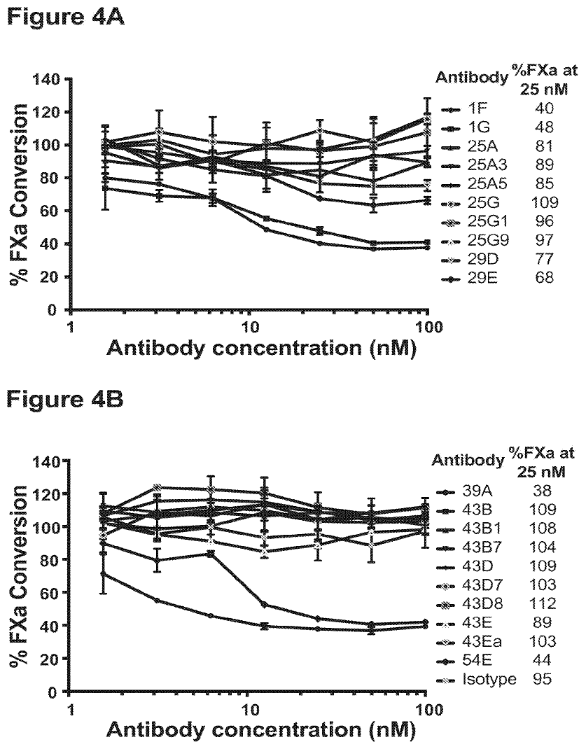

To evaluate the ability of TF:FVIIa to convert FX into FXa in the presence of human antibodies against TF, 5×10⁴ MDA-MB-231 cells (ATCC, Manassas, VA, USA) were plated into tissue culture-treated black 96-well plates (Greiner Bio-One, Monroe, NC, USA). After removal of the cell culture media and addition of a final concentration of 200 nM of FX in a HEPES buffer with 1.5 mM CaCl₂), cells were incubated with a titration of the antibodies for 15 min at 37° C. Upon reconstitution of the binary TF:FVIIa complex with a final concentration of 20 nM of FVIIa, cells were incubated for 5 min at 37° C. After quenching the reaction with ethylenediaminetetraacetic acid (EDTA), generated FXa was measured with 50 μM of SN-7 6-amino-1-naphthalenesulfonamide-based fluorogenic substrate (Haematologic Technologies, Essex Junction, VT, USA) on an Envision plate reader equipped with an Umbelliferone 355 excitation filter, an Umbelliferone 460 emission filter, and a LANCE/DELFIA top mirror (Perkin Elmer, Waltham, MA, USA). FXa conversion percentages (% FXa) in the presence of an anti-TF antibody titration relative to a no-antibody control are summarized in Table 8 and plotted in FIGS. 4A and 4B.

The FXa conversion percentage ranges from about 78% to about 120% in presence of different concentrations of antibodies from groups 25 and 43, including 25A, 25A3, 25G, 25G1, 25G5, 25G9, 43B, 43B1, 43B7, 43D, 43D7, 43D8, 43E, and 43Ea.

This data indicates that anti-TF antibodies from groups 25 and 43 do not inhibit TF:FVIIa mediated FXa conversion from FX. This data also indicates that anti-TF antibodies from groups 25 and 43 have a human TF binding site that is distinct from the human TF binding site bound by FX.

TABLE 8
% FXa conversion

| | % FXa | | | |
|---|---|---|---|---|
| Antibody | 12.5 nM | 25 nM | 50 nM | 100 nM |
| 1F | 49 | 40 | 37 | 38 |
| 1G | 55 | 48 | 41 | 41 |
| 25A | 87 | 81 | 94 | 89 |
| 25A3 | 89 | 89 | 93 | 96 |
| 25A5 | 82 | 85 | 78 | 89 |
| 25G | 99 | 109 | 102 | 116 |
| 25G1 | 101 | 96 | 99 | 108 |
| 25G9 | 98 | 97 | 104 | 117 |
| 29D | 85 | 77 | 75 | 75 |
| 29E | 81 | 68 | 63 | 66 |
| 39A | 39 | 38 | 37 | 39 |
| 43B | 113 | 109 | 105 | 105 |
| 43B1 | 106 | 108 | 108 | 112 |
| 43B7 | 113 | 104 | 108 | 112 |
| 43D | 115 | 109 | 104 | 106 |
| 43D7 | 110 | 103 | 102 | 103 |
| 43D8 | 120 | 112 | 107 | 111 |
| 43E | 85 | 89 | 97 | 98 |
| 43Ea | 108 | 103 | 106 | 101 |
| 54E | 53 | 44 | 41 | 42 |
| 5G9 | 37 | 33 | 30 | 30 |
| Isotype ctrl | 93 | 95 | 89 | 97 |

### Example 6: FVIIa Competition Assay

FVII-Fc conjugates were generated using Alexa Fluor 488 5-sulfo-dichlorophenol esters (ThermoFisher Scientific). Excess Alexa Fluor dye was removed from the conjugate preparations by gel filtration (ThermoFisher Scientific).

To evaluate competition between FVIIa and the human antibodies against TF, TF-positive MDA-MB-231 cells (ATCC, Manassas, VA, USA) were first incubated for 1 hr on ice with a titration of the human antibodies against TF. Subsequently, a final concentration of 20 nM of FVII-Fc conjugated to Alexa488 was added to the antibody cell mixture. After another 1 hr incubation on ice, cells were washed, stained with a viability dye, and analyzed by flow cytometry. The Alexa488 fluorescence data from viable cells was summarized using median fluorescence intensity. FVII-Fc binding was summarized with % FVII-Fc binding=[MFI_{antibody labeled cells}−MFI_{unstained cells}]/[MFI_{IgG1 control labeled cells}−MFI_{unstained cells}].

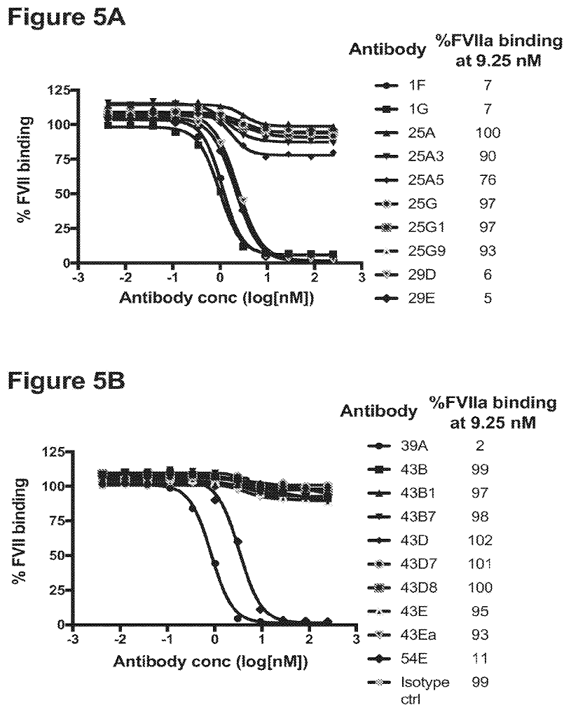

Percentage of FVIIa binding (% FVIIa) in the presence of an anti-TF antibody titration relative to a no-antibody control is summarized in Table 9 and plotted in FIGS. 5A and 5B.

The FVIIa binding percentage ranges from about 76% to about 102% in the presence of antibodies of different concentrations from groups 25 and 43, including 25A, 25A3, 25G, 25G1, 25G5, 25G9, 43B, 43B1, 43B7, 43D, 43D7, 43D8, 43E, and 43Ea.

This data indicates that anti-TF antibodies from groups 25 and 43 do not compete for binding to human TF with FVIIa. This data also indicates that anti-TF antibodies from groups 25 and 43 have a human TF binding site that is distinct from the human TF binding site bound by FVIIa.

TABLE 9
Competition of Anti-TF Antibody with FVIIa

| | % FVIIa | | | |
|---|---|---|---|---|
| Antibody | 9.25 nM | 28 nM | 83 nM | 250 nM |
| 1F | 7 | 7 | 7 | 6 |
| 1G | 7 | 7 | 7 | 6 |
| 25A | 100 | 101 | 97 | 98 |
| 25A3 | 90 | 87 | 88 | 87 |
| 25A5 | 76 | 79 | 77 | 80 |
| 25G | 97 | 96 | 93 | 92 |
| 25G1 | 97 | 93 | 94 | 95 |
| 25G9 | 93 | 93 | 91 | 89 |
| 29D | 6 | 4 | 3 | 3 |
| 29E | 5 | 3 | 2 | 2 |
| 39A | 2 | 2 | 2 | 2 |
| 43B | 99 | 95 | 93 | 91 |
| 43B1 | 97 | 95 | 93 | 91 |
| 43B7 | 98 | 98 | 97 | 97 |
| 43D | 102 | 100 | 98 | 94 |
| 43D7 | 101 | 102 | 100 | 101 |
| 43D8 | 100 | 99 | 98 | 96 |
| 43E | 95 | 92 | 91 | 89 |
| 43Ea | 93 | 91 | 92 | 89 |
| 54E | 11 | 3 | 3 | 2 |
| Isotype | 99 | 98 | 97 | 99 |

### Example 7: TF Signaling Assay

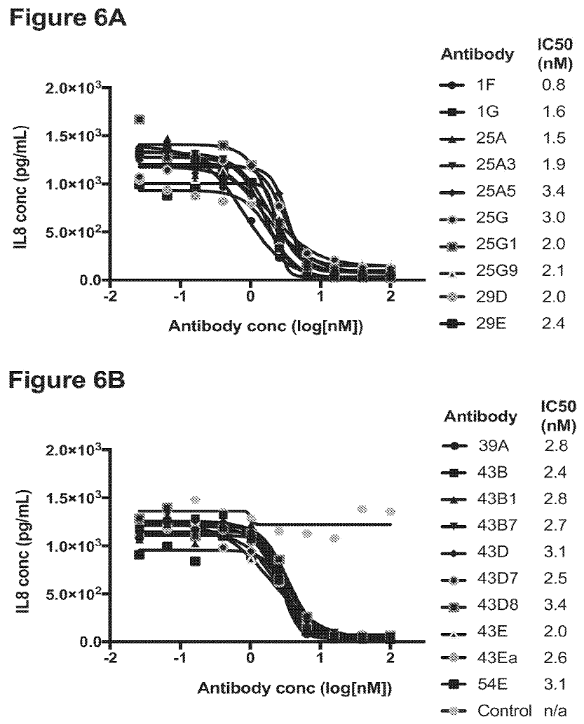

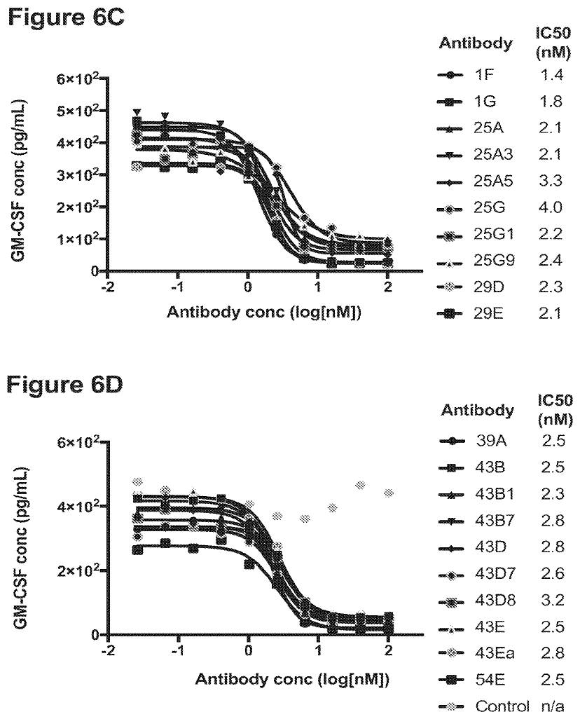

IL-8 and GM-CSF protein levels were measured as described previously in Hjortoe et al., Blood, 2004, 103:3029-3037. TF-positive MDA-MB-231 cells (ATCC, Manassas, VA, USA) that underwent a 2 hr serum starvation with Leibovitz's L-15 medium were incubated with an 8-point 1:2.5 titration starting at 100 nM of anti-TF antibody. After 30 min at 37° C., FVIIa (NovoSeven RT, Novo Nordisk, Bagsvaerd, Denmark) was added to the cells at a final concentration of 20 nM. 5 hr later cell culture supernatants were harvested and analyzed by ELISA for IL8 or GM-CSF as recommended (R&D Biosystems, Minneapolis, MN, USA). A standard curve using recombinant IL8 or GM-CSF (R&D Biosystems, Minneapolis, MN, USA) was used in Prism to calculate cytokine concentration in the cell culture supernatants. Percent IL8 and GM-CSF (% IL8 and % GM-CSF) at reported antibody concentration were calculated relative to a no antibody control. The concentration of IL8 with the anti-TF antibody titration is plotted in FIGS. 6A and 6B and the % IL8 at different antibodies concentrations are shown in Table 10. The concentration of GM-CSF with the anti-TF antibody titration is plotted in FIGS. 6C and 6D and the % IL8 at different antibodies concentrations are shown in Table 11.

IL8 concentrations were reduced by more than 7500 in the presence of the anti-TF antibodies at concentrations greater than or equal to 6.4 nM. GM-CSF concentrations were reduced by more than 6000 in the presence of the anti-TF antibodies at concentrations greater than or equal to 6.4 nM.

This data indicates that all tested anti-TF antibodies inhibit FVIIa-dependent TF signaling.

TABLE 10
Inhibition of IL8

| | % IL8 | | | | |
|---|---|---|---|---|---|
| Antibody | 100 nM | 40 nM | 16 nM | 6.4 nM | 2.56 nM |
| 1F | 2 | 2 | 2 | 3 | 18 |
| 1G | 2 | 2 | 3 | 4 | 26 |
| 25A | 9 | 8 | 10 | 11 | 43 |
| 25A3 | 8 | 8 | 8 | 9 | 47 |
| 25A5 | 6 | 7 | 7 | 14 | 70 |
| 25G | 9 | 10 | 16 | 22 | 60 |
| 25G1 | 9 | 8 | 9 | 12 | 46 |
| 25G9 | 13 | 14 | 15 | 22 | 51 |
| 29D | 1 | 2 | 2 | 6 | 27 |
| 29E | 2 | 2 | 2 | 5 | 33 |
| 39A | 3 | 2 | 2 | 6 | 52 |
| 43B | 4 | 4 | 5 | 11 | 50 |
| 43B1 | 5 | 5 | 6 | 12 | 56 |
| 43B7 | 4 | 4 | 8 | 15 | 55 |
| 43D | 5 | 5 | 7 | 21 | 58 |
| 43D7 | 5 | 4 | 5 | 11 | 48 |
| 43D8 | 5 | 5 | 5 | 21 | 67 |
| 43E | 5 | 5 | 6 | 15 | 49 |
| 43Ea | 6 | 6 | 6 | 14 | 52 |
| 54E | 2 | 2 | 3 | 8 | 48 |
| Control | 106 | 108 | 84 | 88 | 90 |

TABLE 11
Inhibition of GM-CSF

| | % GM-CSF | | | | |
|---|---|---|---|---|---|
| Antibody | 100 nM | 40 nM | 16 nM | 6.4 nM | 2.56 nM |
| 1F | 6 | 6 | 6 | 8 | 27 |
| 1G | 7 | 7 | 7 | 9 | 34 |
| 25A | 22 | 19 | 22 | 24 | 57 |
| 25A3 | 20 | 19 | 19 | 20 | 59 |
| 25A5 | 12 | 15 | 14 | 18 | 72 |
| 25G | 19 | 18 | 32 | 39 | 77 |
| 25G1 | 17 | 16 | 17 | 18 | 48 |
| 25G9 | 25 | 26 | 26 | 34 | 60 |
| 29D | 5 | 6 | 7 | 15 | 38 |
| 29E | 6 | 6 | 5 | 9 | 33 |
| 39A | 7 | 5 | 5 | 8 | 42 |
| 43B | 14 | 13 | 12 | 21 | 59 |
| 43B1 | 11 | 11 | 13 | 16 | 50 |
| 43B7 | 11 | 11 | 13 | 17 | 50 |
| 43D | 12 | 11 | 13 | 24 | 56 |
| 43D7 | 10 | 10 | 9 | 15 | 45 |
| 43D8 | 12 | 11 | 11 | 24 | 57 |
| 43E | 14 | 15 | 15 | 21 | 61 |
| 43Ea | 14 | 15 | 14 | 21 | 65 |
| 54E | 5 | 5 | 5 | 10 | 38 |
| Control | 105 | 111 | 94 | 86 | 88 |

### Example 8: Antibody Competition Assay

Alexa Fluor antibodies were generated using Alexa Fluor 488 5-sulfo-dichlorophenol esters (ThermoFisher Scientific). Excess Alexa Fluor dye was removed from the antibody dye conjugate preparations by gel filtration (ThermoFisher Scientific).

To evaluate competition between a first human antibody against TF and 25A, TF-positive A431 cells (ATCC, Manassas, VA, USA) were first incubated for 1 hr on ice with a titration of the first human antibody against TF. Subsequently, a final concentration of 20 nM of 25A conjugated to Alexa488 was added to the antibody cell mixture. After another 1 hr incubation on ice, cells were washed, stained with a viability dye, and analyzed by flow cytometry. The Alexa488 fluorescence data from viable cells was summarized using median fluorescence intensity. 25A binding was summarized with % 25A binding=[MFI_{antibody labeled cells}−MFI_{unstained cells}]/[MFI_{IgG1 control labeled cells}−MFI_{unstained cells}].

To evaluate competition between a first human antibody against TF and 43Ea, TF-positive A431 cells (ATCC, Manassas, VA, USA) were first incubated for 1 hr on ice with a titration of the first human antibody against TF. Subsequently, a final concentration of 20 nM of 43Ea conjugated to Alexa488 was added to the antibody cell mixture. After another 1 hr incubation on ice, cells were washed, stained with a viability dye, and analyzed by flow cytometry. The Alexa488 fluorescence data from viable cells was summarized using median fluorescence intensity. 43Ea binding was summarized with % 43Ea binding=[MFI_{antibody labeled cells}−MFI_{unstained cells}]/[MFI_{IgG1 control labeled cells}−MFI_{unstained cells}].

% 25A binding and % 43Ea binding are shown in Table 12. Antibodies from group 25 and group 43 reduced the % 25A binding and % 43Ea binding to less than 10%.

This data indicates that antibodies of group 25 and antibodies of group 43 compete with each other for binding to human TF, and may bind the same or an overlapping epitope of human TF.

TABLE 12
Competition of Anti-TF Antibody with Antibody Clone 25A or 43Ea

| Antibody (100 nM) | % 25A binding | % 43Ea binding |
|---|---|---|
| 1F | 95 | 77 |
| 1G | 75 | 58 |
| 25A | 3 | 1 |
| 25G | 7 | 3 |
| 29D | 70 | 64 |
| 29E | 96 | 85 |
| 39A | 99 | 96 |
| 43B | 0 | 0 |
| 43D | 0 | 0 |
| 43E | 0 | 0 |
| 54E | 99 | 96 |
| Isotype | 100 | 100 |

### Example 9: Cell Viability Assay

To evaluate internalization of the anti-TF antibodies, a cytotoxicity assay was conducted. Briefly, cells were plated in 384-well plates (Greiner Bio-One, Monroe, NC, USA) at 4×10³ cells per well in 40 μl of media. Antibodies and secondary anti-human Fc antibodies conjugated to the tubulin inhibitor mono-methyl auristatin F (MMAF) (Moradec, San Diego, CA, USA) were serially diluted starting at 5 and 30 nM, respectively. Plates were incubated for 3 days, followed by lysis in CellTiter-Glo (CTG) assay reagent (Promega, Madison, WI, USA). CTG luminescence was measured on an Envision plate reader and the mean and standard deviation of 4 replicates graphed in Prism. For each anti-TF antibody, the IC₅₀ and its associated 95% confidence interval were calculated in Prism using a 4-parameter binding model.

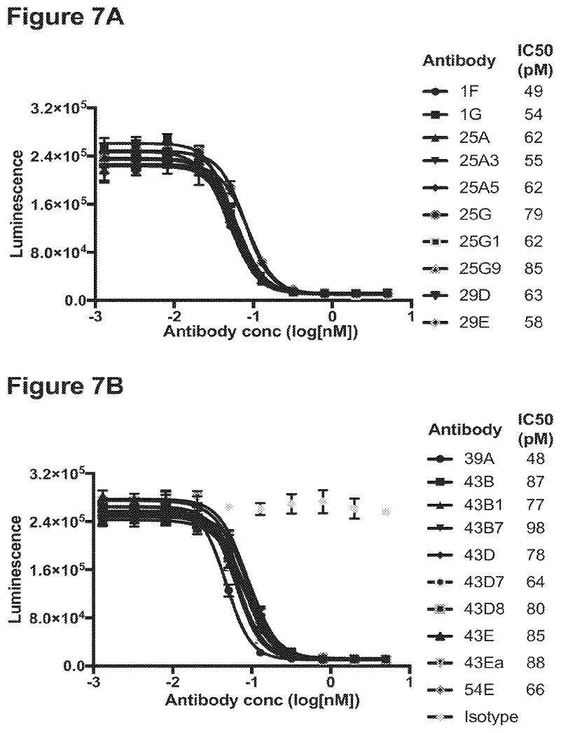

FIGS. 7A and 7B show the cell viability as indicated by the level of luminescence and the calculated IC₅₀.

This data indicates that all anti-TF antibodies tested from groups 1, 25, 29, 39, 43, and 54 were effective in reducing the viability of TF-positive A431 cells.

### Example 10: Thrombin Generation Assay (TGA)

The TGA assay was performed using the calibrated-automated-thrombogram (CAT) instrument manufactured and distributed by STAGO. The test method design was equivalent to a standard CAT assay measurement, except that the plasma source was normal pooled plasma (NPP) in citrate supplemented with corn trypsin inhibitor (citrate/CTI). The anti-TF antibodies were titrated at 0, 10, 50 and 100 nM and mixed with normal pooled plasma (NPP) collected in 11 mM citrate supplemented with 100 microgram/mL of corn trypsin inhibitor (citrate/CTI). Relipidated TF was added to a 96-well assay plate, followed by addition of the antibody/NPP mixture. After a 10-min incubation or directly after combining the relipidated TF with antibody/NPP, thrombin generation was initiated by the addition of calcium and the thrombin substrate. The STAGO software was used to report the following parameters: Peak IIa (highest thrombin concentration generated [nM]); Lag Time (time to IIa generation [min]); ETP (endogenous thrombin potential, area under the curve [nM×min]); and ttPeak (time to Peak IIa [min]). Percent peak thrombin generation (% Peak IIa) and percent endogenous thrombin potential (% ETP) in the presence of each antibody relative to a no antibody plasma control on the same plate were also reported.

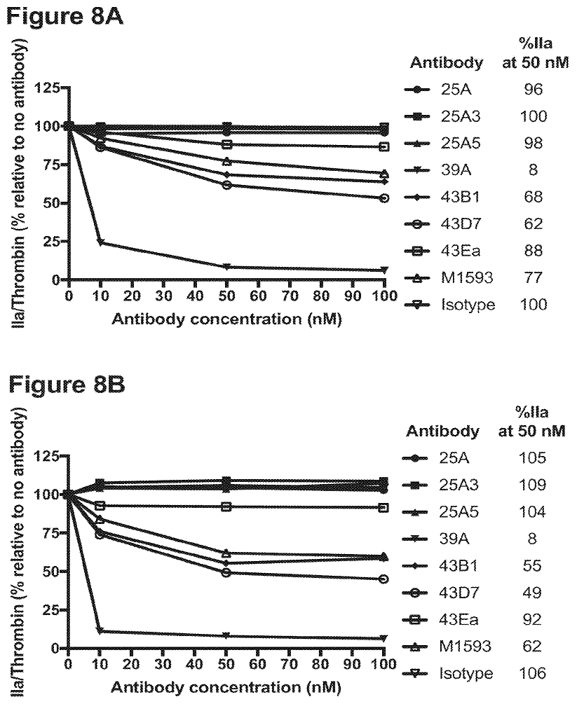

The Peak IIa, Lag Time, ETP, ttPeak, % Peak IIa, and % ETP in the presence of each antibody selected from 25A, 25A3, 25A5, 39A, 43B1, 43D7, 43Ea, and M1593 without antibody incubation prior to addition of calcium and thrombin substrate are shown in Table 37. The Peak IIa, Lag Time, ETP, ttPeak, % Peak IIa, and % ETP in the presence of each antibody selected from 25A, 25A3, 25A5, 39A, 43B1, 43D7, 43Ea, and M1593 with 10 min antibody incubation prior to addition of calcium and thrombin substrate are shown in Table 38. The % Peak IIa in the presence of titrations of anti-TF antibodies without antibody incubation prior to addition of calcium and thrombin substrate is plotted in FIG. 8A. The % Peak IIa in the presence of titrations of anti-TF antibodies with 10 min antibody incubation prior to addition of calcium and thrombin substrate is plotted in FIG. 8B. The M1593 antibody has a V_{H} sequence of SEQ ID NO:821 and VL sequence of SEQ ID NO:822.

The % Peak IIa is 95% or greater in the presence of antibodies from group 25, including 25A, 25A3, and 25A5 without antibody pre-incubation. The % Peak IIa is 100% or greater in the presence of antibodies from group 25, including 25A, 25A3, and 25A5 with 10 min antibody pre-incubation. The % ETP is 99% or greater in the presence of the tested antibodies from group 25.

The % Peak IIa is greater than 50% but equal to or less than 96% in the presence of antibodies from group 43, including 43B1, 43D7, and 43Ea and anti-TF antibody M1593 without antibody pre-incubation. The % Peak IIa is greater than 40% but equal to or less than 93% in the presence of antibodies from group 43, including 43B1, 43D7, and 43Ea and anti-TF antibody M1593 with 10 min antibody pre-incubation. The % ETP is 92% or greater in the presence of the tested antibodies from group 43 and M1593 antibody.

This data indicates that antibodies from groups 25 and 43 allow normal thrombin generation, and therefore are not inhibitors of thrombin generation. The percent peak thrombin generation (% Peak IIa) is greater in the presence of antibodies of group 25 compared to antibodies of group 43 and M1593 antibody.

TABLE 37
Thrombin Generation Assay without Antibody Pre-Incubation

| | | Ab | Peak | Lag | | | % | |
|---|---|---|---|---|---|---|---|---|
| | | conc. | IIa | Time | ETP | ttPeak | Peak | % |
| Plate | Antibody | (nM) | (nM) | (mm) | (nM · min) | (mm) | IIa | ETP |
| 3 | 25A | 100 | 334 | 5.0 | 2390 | 8.7 | 96 | 105 |
| | | 50 | 335 | 5.0 | 2380 | 8.7 | 96 | 104 |
| | | 10 | 333 | 5.0 | 2387 | 8.6 | 95 | 104 |
| 3 | 25A3 | 100 | 343 | 5.0 | 2405 | 8.4 | 98 | 105 |
| | | 50 | 349 | 5.0 | 2433 | 8.4 | 100 | 106 |
| | | 10 | 350 | 5.0 | 2426 | 8.0 | 100 | 106 |
| 3 | 25A5 | 100 | 342 | 5.1 | 2393 | 8.5 | 98 | 105 |
| | | 50 | 344 | 4.8 | 2317 | 8.1 | 98 | 101 |
| | | 10 | 343 | 4.7 | 2270 | 8.0 | 98 | 99 |
| 3 | 39A | 100 | 22 | 38.1 | * | 48.3 | 6 | * |
| | | 50 | 29 | 33.1 | * | 43.2 | 8 | * |
| | | 10 | 84 | 12.4 | 1332 | 20.7 | 24 | 58 |
| 3 | 43B1 | 100 | 223 | 4.8 | 2111 | 10.0 | 64 | 92 |
| | | 50 | 239 | 4.9 | 2134 | 9.9 | 68 | 93 |
| | | 10 | 303 | 5.1 | 2318 | 9.1 | 87 | 101 |
| 3 | 43D7 | 100 | 186 | 5.6 | 2105 | 12.2 | 53 | 92 |
| | | 50 | 216 | 5.5 | 2183 | 11.3 | 62 | 96 |
| | | 10 | 301 | 5.4 | 2338 | 9.3 | 86 | 102 |
| 3 | 43Ea | 100 | 302 | 5.1 | 2347 | 9.1 | 87 | 103 |
| | | 50 | 308 | 5.1 | 2392 | 8.8 | 88 | 105 |
| | | 10 | 336 | 4.5 | 2305 | 7.8 | 96 | 101 |
| 3 | M1593 | 100 | 242 | 5.1 | 2235 | 10.4 | 69 | 98 |
| | | 50 | 270 | 5.1 | 2282 | 9.8 | 77 | 100 |
| | | 10 | 322 | 5.1 | 2368 | 8.8 | 92 | 104 |
| 3 | Isotype | 100 | 347 | 5.0 | 2319 | 8.1 | 99 | 101 |
| | | 50 | 348 | 5.0 | 2324 | 8.1 | 100 | 102 |
| | | 10 | 348 | 5.0 | 2326 | 8.3 | 100 | 102 |
| 3 | Plasma | NA | 349 | 4.7 | 2285 | 7.7 | 100 | 100 |
| | ctrl. | | | | | | | |

* Groups with “No Tail Found” Errors when the software cannot calculate the ETP.

TABLE 38
Thrombin Generation Assay with 10 min Antibody Pre-Incubation

| | | Ab | Peak | Lag | | | % | |
|---|---|---|---|---|---|---|---|---|
| | | conc. | IIa | Time | ETP | ttPeak | Peak | % |
| Plate | Antibody | (nM) | (nM) | (mm) | (nM · min) | (mm) | IIa | ETP |
| 3 | 25A | 100 | 274 | 3.3 | 1879 | 7.0 | 103 | 106 |
| | | 50 | 279 | 3.3 | 1876 | 7.0 | 105 | 106 |
| | | 10 | 280 | 3.6 | 1872 | 7.0 | 105 | 106 |
| 3 | 25A3 | 100 | 290 | 3.4 | 1906 | 6.8 | 109 | 108 |
| | | 50 | 291 | 3.6 | 1925 | 6.8 | 109 | 109 |
| | | 10 | 287 | 3.3 | 1886 | 6.8 | 108 | 107 |
| 3 | 25A5 | 100 | 286 | 3.7 | 1883 | 7.0 | 107 | 107 |
| | | 50 | 277 | 3.7 | 1803 | 7.0 | 104 | 102 |
| | | 10 | 278 | 3.7 | 1808 | 7.0 | 104 | 102 |
| 3 | 39A | 100 | 17 | 32.1 | * | 43.2 | 6 | * |
| | | 50 | 21 | 29.0 | * | 39.7 | 8 | * |
| | | 10 | 30 | 20.9 | * | 30.8 | 11 | * |
| 3 | 43B1 | 100 | 156 | 3.6 | 1701 | 9.3 | 58 | 96 |
| | | 50 | 148 | 3.3 | 1667 | 9.6 | 55 | 94 |
| | | 10 | 203 | 3.7 | 1776 | 8.2 | 76 | 101 |
| 3 | 43D7 | 100 | 120 | 3.7 | 1633 | 10.8 | 45 | 92 |
| | | 50 | 131 | 3.7 | 1724 | 10.4 | 49 | 98 |
| | | 10 | 197 | 3.7 | 1784 | 8.8 | 74 | 101 |
| 3 | 43Ea | 100 | 244 | 3.3 | 1817 | 7.3 | 91 | 103 |
| | | 50 | 246 | 3.3 | 1833 | 7.3 | 92 | 104 |
| | | 10 | 247 | 3.3 | 1779 | 7.1 | 93 | 101 |
| 3 | M1593 | 100 | 160 | 3.7 | 1737 | 9.4 | 60 | 98 |
| | | 50 | 165 | 3.7 | 1739 | 9.3 | 62 | 99 |
| | | 10 | 224 | 3.7 | 1807 | 8.0 | 84 | 102 |
| 3 | Isotype | 100 | 279 | 3.7 | 1829 | 7.2 | 105 | 104 |
| | | 50 | 283 | 3.7 | 1839 | 7.0 | 106 | 104 |
| | | 10 | 279 | 3.7 | 1814 | 7.1 | 105 | 103 |
| 3 | Plasma | NA | 267 | 3.7 | 1766 | 7.2 | 100 | 100 |
| | ctrl. | | | | | | | |

* Groups with “No Tail Found” Errors when the software cannot calculate the ETP.

### Example 11: Synthesis of Antibody-Drug Conjugates (ADCs)

Antibody-Drug Conjugates (ADCs) were synthesized as described in Behrens et al., Mol Pharm, 2015, 12:3986-98. 5 mg/mL of antibody in phosphate-buffered saline (PBS), pH 7.4 was reduced with 2.5 molar equivalents of Tris(2-carboxyehtyl)phosphine. After 2 hr at 37° C., the partially reduced antibody was cooled to room temperature and conjugated for 1 hr to 3 to 5 molar equivalents of MC-vc-PAB-MMAE (maleimidocaproyl-valine-citrulline-p-aminobenzoyloxycarbonyl-monomethyl auristatin E). The reaction was buffer exchanged into PBS to remove small molecular weight reagents. The drug-antibody ratio (DAR) of the resulting ADCs was 3-4. The DAR was determined with the following formula: Absorbance (248 nm)/Absorbance (280 nm)=(n×Ex_{PAB[248 nm]}+Ex_{antibody[248 nm]})/(n×Ex_{PAB[280 nm]}+Ex_{antibody[280 nm]}) with n as a variable for the DAR and Ex as the extinction coefficients of PAB and the antibody. Hydrophobic interaction chromatography and size exclusion chromatography were used to corroborate the absorbance-based DAR estimation and to ensure the ADC preparation was at least 95% monomeric, respectively.

### Example 12: Cytotoxicity Assays of Antibody-Drug Conjugates (ADCs)

To evaluate cytotoxicity of ADCs, TF-positive A431 and HPAF-II cells were plated in 384-well plates (Greiner Bio-One, Monroe, NC, USA) at 4×10³ cells per well in 40 μL of media. Anti-TF antibodies conjugated to MC-vc-PAB-MMAE were serially diluted starting at 5 nM. Plates were incubated for 3 to 4 days, followed by lysis in CellTiter-Glo (CTG) assay reagent (Promega, Madison, WI, USA). CTG luminescence was measured on an Envision plate reader and the mean and standard deviation of 4 replicates were graphed in Prism. For each ADC, the IC₅₀ and its associated 95% confidence interval were calculated in Prism using a 4-parameter binding model.

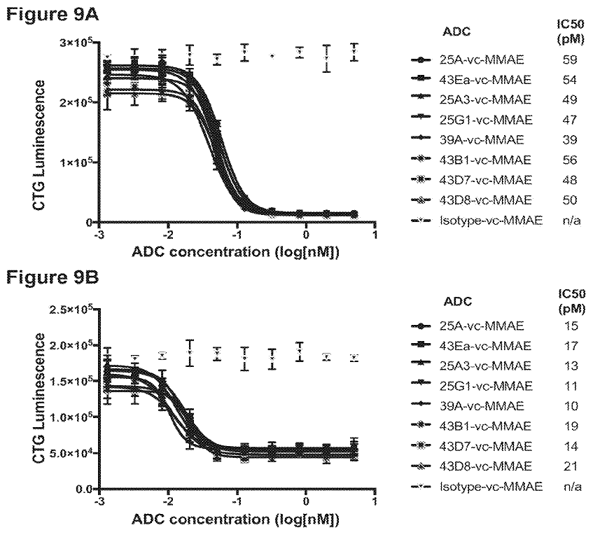

FIGS. 9A and 9B show the cell viability as indicated by CTG luminescence and the calculated IC₅₀ in TF-positive A431 and HPAF-II cells, respectively. ADCs comprising anti-TF antibodies from groups 25, 43, and 39 conjugated to MC-vc-PAB-MMAE resulted in cytotoxicity in TF-positive A431 and HPAF-II cells.

This data indicates that anti-TF antibody-drug conjugates reduced the viability of TF-positive cells in vitro.

### Example 13: Xenograft Cell Line Studies

Xenograft studies in immune compromised mice were performed to evaluate the efficacy of the ADCs in vivo. The TF-positive A431 epidermoid carcinoma and the HPAF-II pancreatic carcinoma xenografts were implanted subcutaneously in the flank of athymic nude mice (Charles River Laboratories, Wilmington, MA). Animals were randomized when tumors reached an average size of 150-200 mm³ and treated with 5 mg/kg of the indicated ADC or vehicle (PBS) intraperitoneally (i.p.) once weekly for 3 weeks. Body weight and tumor size assessments were performed bi-weekly. Animals were removed from study and euthanized once tumor size reached 1200 mm³ or skin ulceration was evident.

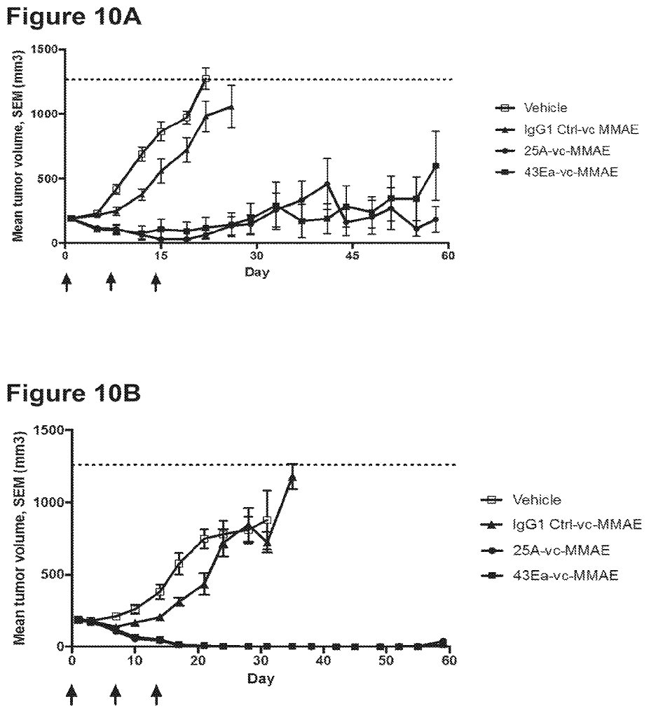

FIGS. 10A and 10B show the tumor size of vehicle-treated, IgG1 ADC-treated, and anti-TF ADC-treated groups in the TF-positive A431 epidermoid carcinoma and the HPAF-II pancreatic carcinoma xenograft models, respectively. ADCs comprising anti-TF antibodies 25A and 43Ea conjugated to MC-vc-PAB-MMAE decreased the tumor size in both xenograft models compared to the vehicle-treated or IgG1 ADC-treated groups.

This data indicates that anti-TF antibody-drug conjugates 25A-vc-MMAE and 43Ea-vc-MMAE were effective in reducing the tumor size in vivo.

### Example 14: Studies of Patient-Derived Xenograft (PDX) Model

A TF-positive head and neck cancer patient-derived xenograft model was generated in athymic nude mice (Envigo, Indianapolis, IN) to further evaluate the efficacy of the ADCs in vivo. Tumors were passaged in stock animals and harvested for re-implantation. Study animals were implanted unilaterally on the left flank with tumor fragments and were randomized to treatment group when tumors reached an average size of 150-200 mm³. Animals were treated with 5 mg/kg of the indicated ADC intraperitoneally (i.p.) once weekly for 2 weeks. Body weight and tumor size assessments were performed bi-weekly. Animals were removed from study and euthanized once tumor size reached 1200 mm³ or skin ulceration was evident.

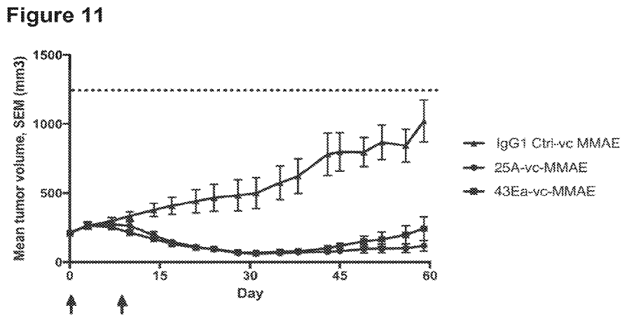

FIG. 11 shows the tumor size of IgG1 ADC-treated and anti-TF ADC-treated groups in the head and neck cancer patient-derived xenograft model. ADCs comprising anti-TF antibodies 25A and 43Ea conjugated to MC-vc-PAB-MMAE decreased the tumor size in the cancer patient-derived xenograft model compared to the IgG1 ADC-treated group.

This data indicates that anti-TF antibody-drug conjugates 25A-vc-MMAE and 43Ea-vc-MMAE were effective in reducing the tumor size in a cancer patient-derived xenograft model in vivo.

### Example 15: Binding Affinity Assay for Pig TF

The ability of certain antibodies was tested for binding to pig TF. For pig TF Biacore-based measurements, a given anti-TF antibody was captured by an anti-human IgG antibody covalently coupled to a CM5 chip (GE Healthcare Bio-Sciences). Association between the anti-TF antibodies and a five-point three-fold titration of pig TF-His starting at 100 nM was measured for 180 to 240 sec. Subsequently, dissociation between the anti-TF antibody and TF-His was measured for 1800 sec. Kinetic data was analyzed and fitted globally using a 1:1 binding model. The K_{D} values of the indicated TF antibodies measured by the Biacore-based experiments are shown in Table 40.

As shown in Table 40, anti-hTF antibodies from groups 25 and 43, 25G9 and 43D8, exhibit binding activity and cross-reactivity to pig TF.

TABLE 40
Antibody kinetics for pig TF

| Ab | Pig K_{D} (nM) [standard deviation] | |
|---|---|---|
| 1G | no binding* | |
| 29D | no binding* | |
| 25G9 | 3.31 | [0.08] |
| 43D8 | 12.9 | [0.03] |

no binding*: no binding to weak binding, with no reportable K_{D}

### Example 16: Cell-Based Binding Assay

Human TF-positive cancer cell lines A431 and MDA-MB-231 and Macaca mulatta TF-positive cell line RF/6A were obtained from the American Tissue Culture Collection (ATCC, Manassas, VA, USA) and were maintained as recommended.

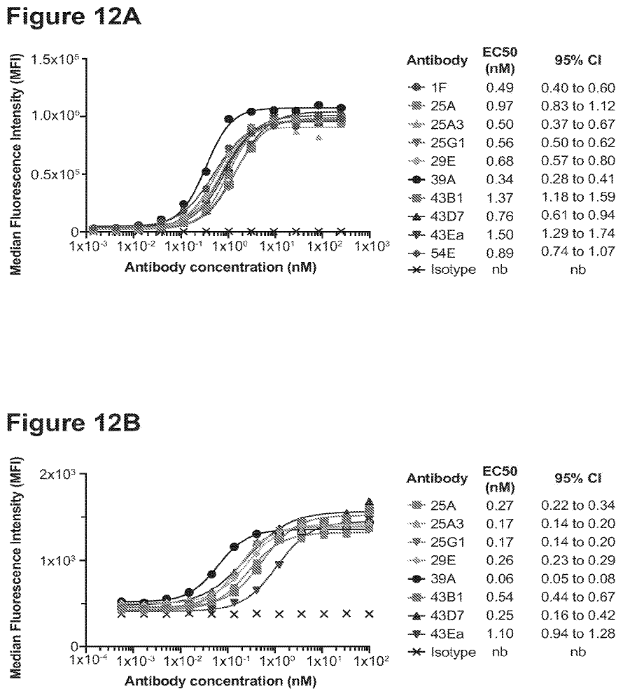

Cell-based antibody binding was assessed as previously described in Liao-Chan et al., PLoS One, 2015, 10:e0124708, which is incorporated by reference in its entirety. 1.2×10⁵ cells collected with Cellstripper (Mediatech, Manassas, VA, USA) were incubated with a twelve-point 1:3 dilution titration of anti-human TF IgG1 antibody starting at 250 nM or 100 nM for 2 hr on ice. After 2 washes, cells labeled with IgG1 antibody were incubated for 30 min on ice with 150 nM of Goat Phycoerythrin (PE) F(ab′)₂ fragment goat anti-human IgG, Fcγ fragment specific (Jackson ImmunoResearch, West Grove, PA, USA) or FITC-labeled F(ab′)₂ fragment goat anti-human kappa (SouthemfBiotech, Birmingham, AL, USA), respectively. After 2 washes, dead cells were labeled with TO-PRO-3 Iodide (ThermoFisher Scientific) and samples were analyzed on a CytoFLEX flow cytometer (Beckman Coulter, Brea, CA, USA) or Novocyte flow cytometer (ACEA Biosciences, San Diego, CA, USA). The median fluorescence intensities (MFIs) at each dilution were plotted and cell EC₅₀'s were derived using a 4-parameter binding model in Prism (GraphPad, La Jolla, CA, USA). Antibodies that does not substantially affect FX conversion (i.e. 25A, 25A3, 25G1, 43B1, 43D7 and 43Ea) and antibodies that inhibited FX conversion by more than 50% (i.e. 1F, 29E, 39A and 54E) were included in the assay. The results of binding of anti-TF antibodies to human TF-positive A431 cells are shown in FIG. 12A. The results of binding of anti-TF antibodies to human TF-positive MDA-MB-231 cells are shown in FIG. 12B.

All tested anti-hTF antibodies in FIG. 12A exhibit high affinity to human TF-positive A431 cells with an EC₅₀ ranging from about 1.50 nM to about 0.34 nM. An IgG1 isotype control did not bind A431 cells (no binding, nb). All tested anti-hTF antibodies in FIG. 12B exhibit high affinity to human TF-positive MDA-MB-231 cells with an EC₅₀ ranging from about 1.50 nM to about 0.06 nM. An IgG1 isotype control did not bind MDA-MB-231 cells (no binding, nb).

As described in Example 2 and shown in Table 5, the binding affinity of anti-hTF antibodies was evaluated on TF from cynomolgus monkey (Macaca fascicularis). The protein sequences of Macaca fascicularis TF and Macaca mulatta TF are identical. The binding of the TF-specific antibodies to cynomolgus monkey was confirmed using the Macaca mulatta RF/6A cell line as shown in Table 42. All tested anti-hTF antibodies exhibit high affinity to TF-positive Macaca mulatta RF/6A cells with an EC₅₀ ranging from about 1.28 nM to about 0.17 nM. The ability of the anti-TF antibodies to bind to cynomolgus monkey is advantageous for toxicology studies of these antibodies with nonhuman primate models.

TABLE 42
Binding of anti-TF antibodies to
Macaca mulatta RF/6A cells

| | RF/6A | RF/6A |
|---|---|---|
| Ab | EC50 (nM) | 95% CI |
| 1F | 0.17 | 0.14 to 0.21 |
| 25A | 0.43 | 0.37 to 0.50 |
| 25A3 | 0.27 | 0.24 to 0.30 |
| 25G1 | 0.27 | 0.23 to 0.32 |
| 29E | 0.53 | 0.46 to 0.61 |
| 39A | 0.27 | 0.23 to 0.32 |
| 43B1 | 0.47 | 0.40 to 0.55 |
| 43D7 | 0.41 | 0.35 to 0.49 |
| 43Ea | 0.92 | 0.83 to 1.01 |
| 54E | 1.28 | 1.16 to 1.41 |

### Example 17: Binding Assay to E. Coli-Derived TF

E. coli-derived TF was expressed as a fusion between the OmpA signal sequence and TF ECD-His6, and purified by affinity and anion exchange chromatography. The binding of anti-TF antibodies 1F, 25A, 25A3, 25G1, 29E, 39A, 43B1, 43D7, 43Ea, and 54E to Expi293- or E. coli-derived TF was determined by protein ELISA studies. Plates coated with Expi293- or E. coli-derived TF-His were incubated with increasing concentrations of antibodies. After incubation with an HRP-conjugated secondary antibody (Jackson Immunoresearch), luminescence data were obtained and used to calculate an EC₅₀ with 95% confidence intervals using Prism. The EC₅₀'s and 95% confidence intervals of the antibodies are listed in Table 43.

TABLE 43
Binding of anti-TF antibodies to Expi293- or E. coli-derived TF

| | Expi293- | Expi293- | | |
|---|---|---|---|---|
| | derived TF | derived TF | E. coli-derived | E.coli-derived |
| | protein EC50 | protein 95% | TF protein | TF protein |
| Ab | (nM) | CI | EC50 (nM) | 95% CI |
| 1F | 0.41 | 0.37 to 0.46 | 0.32 | 0.30 to 0.34 |
| 25A | 0.54 | 0.49 to 0.60 | 0.35 | 0.30 to 0.41 |
| 25A3 | 0.47 | 0.39 to 0.56 | 0.36 | 0.31 to 0.42 |
| 25G1 | 0.42 | 0.36 to 0.47 | 0.31 | 0.29 to 0.33 |
| 29E | 0.98 | 0.78 to 1.24 | 0.68 | 0.39 to 1.26 |
| 39A | 0.45 | 0.39 to 0.53 | 0.34 | 0.28 to 0.40 |
| 43B1 | 0.57 | 0.53 to 0.61 | 0.39 | 0.34 to 0.44 |
| 43D7 | 0.71 | 0.62 to 0.80 | 0.43 | 0.35 to 0.53 |
| 43Ea | 0.74 | 0.68 to 0.81 | 0.46 | 0.40 to 0.53 |
| 54E | 0.96 | 0.73 to 1.29 | 0.38 | 0.22 to 0.62 |

All tested anti-hTF antibodies exhibit high affinity to E. coli-derived TF with an EC₅₀ ranging from about 0.68 nM to about 0.31 nM, which is comparable to the binding affinity of the antibodies to Expi293-derived TF (about 0.98 nM to 0.41 nM). These results indicate that although the anti-TF antibodies were selected against glycosylated TF from a human cell line, the antibodies can bind to E. coli-derived TF with similar affinity when measured by protein ELISA.

### Example 18: Thrombin Generation Assay (TGA)

TGA assay was performed using the calibrated-automated-thrombogram (CAT) instrument manufactured and distributed by STAGO (Diagnostica Stago SAS, Asnieres sur Seine, France). See Samama et al., Thromb Res, 2012, 129:e77-82, which is incorporated by reference in its entirety. The test method design was equivalent to a standard CAT assay measurement, except that the plasma source was normal pooled plasma (NPP) collected in 11 mM citrate supplemented with 100 μg/mL of corn trypsin inhibitor (citrate/CTI). The anti-TF antibodies were titrated at 0, 10, 50 and 100 nM and mixed with NPP in citrate/CTI. Relipidated TF was added to a 96-well assay plate, followed by addition of the antibody/NPP mixture. After a 10-min incubation or directly after combining the relipidated TF with antibody/NPP, thrombin generation was initiated by the addition of calcium and the thrombin substrate. The STAGO software was used to report the following parameters: Peak IIa (highest thrombin concentration generated on the thrombin generation curve [nM]); Lag Time (time from assay start to the moment 10 nM of thrombin is formed [min]); ETP (endogenous thrombin potential, area under the curve [nM×min]); and ttPeak (time from assay start to Peak IIa [min]). Percent peak thrombin generation (% Peak IIa), percent endogenous thrombin potential (% ETP), and percent ttPeak (% ttPeak) in the presence of each antibody relative to a no-antibody plasma control on the same plate were also reported. As used herein, the term “thrombin generation assay” (TGA) refers to the TGA used in this example.

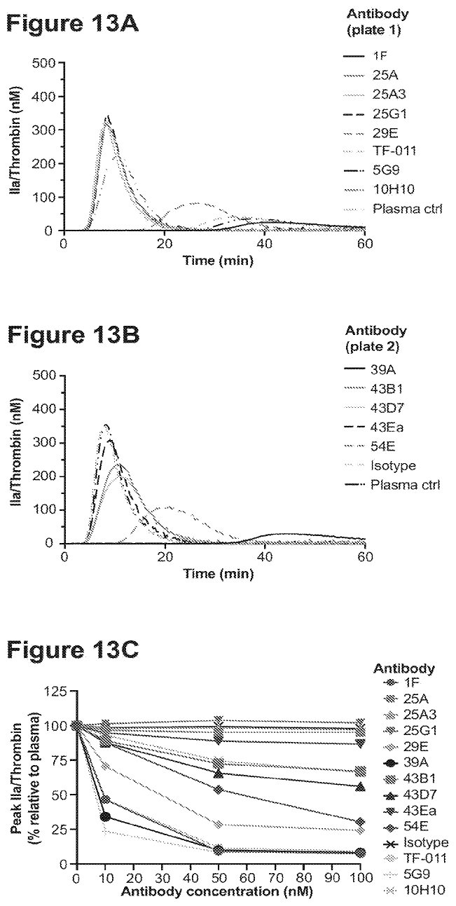

The Peak IIa, Lag Time, ETP, ttPeak, % Peak IIa, % ETP, and % ttPeak in the presence of each antibody selected from 1F, 25A, 25A3, 25G1, 29E, 39A, 43B1, 43D7, 43Ea, 54E, TF-011, 5G9, and 10H10 without antibody incubation prior to addition of calcium and thrombin substrate are shown in Table 44. The Peak IIa, Lag Time, ETP, ttPeak, % Peak IIa, % ETP, and % ttPeak in the presence of each antibody selected from 1F, 25A, 25A3, 25G1, 29E, 39A, 43B1, 43D7, 43Ea, 54E, TF-011, 5G9, and 10H10 with 10 min antibody incubation prior to addition of calcium and thrombin substrate are shown in Table 45. The thrombin generation curve in the presence of 100 nM anti-TF antibody without antibody pre-incubation is plotted in FIGS. 13A and 13B. The Peak thrombin concentration in the presence of titrations of anti-TF antibodies without antibody pre-incubation is plotted in FIG. 13C.

As shown in FIGS. 13A, 13B, and 13C and Table 44, under the conditions without antibody pre-incubation, at the 100 nM antibody concentration, 1F, 29E, 39A, 54E diminished the peak IIa concentration by 92, 76, 91 and 70%, respectively. Similarly, 100 nM of 5G9 and TF-011 inhibited peak IIa concentration by 92% and 91%, respectively. Severely reduced thrombin generation in the presence of the two highest concentrations of 1F, 39A, 5G9 and TF-011 hampered endogenous thrombin generation (ETP) calculations and increased time to Peak IIa/thrombin generation (ttPeak) by at least 284% and 353% at 50 nM and 100 nM respectively. In contrast, antibodies from group 25 did not impact the peak IIa concentration or ttPeak by more than 9%. Group 43 antibodies and 10H10 exhibited mild interference with the peak IIa concentration: 100 nM of 43B1, 43D7, 43Ea and 10H10 reduced the peak IIa concentration by 33, 44, 13 and 34%, respectively. In addition, 100 nM of 43B1, 43D7 and 10H10 showed at least a 29% increase in ttPeak. However, the observed decline in peak IIa concentration and delayed ttPeak for group 43 antibodies and 10H10 did not result in more than a 10% decline in the ETP.

Similar results are shown in Table 45 under the conditions with 10 min antibody pre-incubation. At the 100 nM antibody concentration, 1F, 29E, 39A, 54E diminished the peak IIa concentration by 93, 72, 93 and 87%, respectively. Similarly, 100 nM of 5G9 and TF-011 inhibited peak IIa concentration by 92% and 91%, respectively. Severely reduced thrombin generation in the presence of the two highest concentrations of 1F, 39A, 54E and TF-011 and all tested concentrations of 5G9 hampered endogenous thrombin generation (ETP) calculations and increased time to Peak IIa/thrombin generation (ttPeak) by at least 303% and 371% at 50 nM and 100 nM respectively. In contrast, antibodies from group 25 did not decrease the peak IIa concentration or increase ttPeak. Group 43 antibodies and 10H10 exhibited mild interference with the peak IIa concentration: 100 nM of 43B1, 43D7, 43Ea and 10H10 reduced the peak IIa concentration by 41, 56, 13 and 48%, respectively. In addition, 100 nM of 43B1, 43D7 and 10H10 showed at least a 33% increase in ttPeak. However, the observed decline in peak IIa concentration and delayed ttPeak for group 43 antibodies and 10H10 did not result in more than an 11% decline in the ETP.

Overall, these results indicate that group 25 antibodies are completely inert in the penultimate step of the coagulation cascade when all three TGA parameters (ETP, Peak IIa concentration and ttPeak) are taken into consideration.

TABLE 44
Thrombin Generation Assay without Antibody Pre-Incubation

| | | | Peak | Lag | | | | | |
|---|---|---|---|---|---|---|---|---|---|
| | | Ab | IIa | Time | ETP | ttPeak | % | | |
| | | conc. | [nM] | [min] | [nM · min] | [min] | Peak | % | % |
| Plate | Sample | (nM) | (SD) | (SD) | (SD) | (SD) | IIa | ETP | ttPeak |
| 1 | 1F | 100 | 25 (1) | 31 (1) | * | 41 (0.7) | 8 | * | 419 |
| | | 50 | 31 (0) | 25.6 (0.3) | * | 35.3 (0.3) | 9 | * | 347 |
| | | 10 | 155 (1) | 8.2 (0.2) | 1738 (25) | 14.9 (0.2) | 47 | 86 | 89 |
| 1 | 25A | 100 | 317 (6) | 5.2 (0.2) | 2134 (28) | 8.6 (0.2) | 95 | 105 | 9 |
| | | 50 | 317 (2) | 5.2 (0.2) | 2122 (30) | 8.6 (0.2) | 95 | 105 | 9 |
| | | 10 | 322 (2) | 5 (0) | 2108 (29) | 8.2 (0.2) | 97 | 104 | 4 |
| 1 | 25A3 | 100 | 323 (1) | 4.6 (0.2) | 2031 (19) | 7.9 (0.2) | 97 | 100 | 0 |
| | | 50 | 328 (2) | 4.7 (0) | 2080 (23) | 8 (0) | 98 | 103 | 1 |
| | | 10 | 326 (4) | 5.3 (0) | 2152 (14) | 8.4 (0.2) | 98 | 106 | 6 |
| 1 | 25G1 | 100 | 340 (3) | 5.3 (0) | 2160 (27) | 8.3 (0) | 102 | 107 | 5 |
| | | 50 | 346 (6) | 5.1 (0.2) | 2221 (40) | 8.2 (0.2) | 104 | 110 | 4 |
| | | 10 | 337 (1) | 4.7 (0) | 2061 (34) | 7.8 (0.2) | 101 | 102 | −1 |
| 1 | 29E | 100 | 81 (0) | 17.1 (0.2) | 1257 (18) | 26.2 (0.2) | 24 | 62 | 232 |
| | | 50 | 95 (1) | 14.1 (0.2) | 1365 (26) | 22.6 (0.4) | 29 | 67 | 186 |
| | | 10 | 235 (3) | 7 (0) | 1926 (9) | 11.7 (0) | 71 | 95 | 48 |
| 1 | Isotype | 100 | 326 (3) | 5.3 (0) | 2132 (13) | 8.6 (0.2) | 98 | 105 | 9 |
| | | 50 | 331 (3) | 5.3 (0) | 2177 (19) | 8.3 (0) | 99 | 108 | 5 |
| | | 10 | 328 (4) | 5.3 (0) | 2129 (26) | 8.4 (0.2) | 98 | 105 | 6 |
| 1 | TF-011 | 100 | 30 (1) | 26 (0.3) | * | 35.8 (0.2) | 9 | * | 353 |
| | | 50 | 39 (3) | 21.3 (0.5) | * | 30.3 (1.1) | 12 | * | 284 |
| | | 10 | 156 (7) | 8 (0) | 1714 (41) | 14.7 (0.5) | 47 | 85 | 86 |
| 1 | 5G9 | 100 | 27 (1) | 29.9 (0.4) | * | 39.6 (0.4) | 8 | * | 401 |
| | | 50 | 28 (0) | 25.1 (0.4) | * | 34.6 (0.2) | 8 | * | 338 |
| | | 10 | 79 (1) | 10.4 (0.2) | 1176 (16) | 18.6 (0.2) | 24 | 58 | 135 |
| 1 | 10H10 | 100 | 221 (4) | 5.2 (0.2) | 1945 (37) | 10.2 (0.2) | 66 | 96 | 29 |
| | | 50 | 248 (3) | 5.2 (0.2) | 1978 (32) | 9.8 (0.3) | 74 | 98 | 24 |
| | | 10 | 310 (2) | 5.2 (0.2) | 2036 (33) | 8.6 (0.2) | 93 | 101 | 9 |
| 1 | Plasma | NA | 333 (0) | 4.7 (0) | 2023 (30) | 7.9 (0.2) | 100 | 100 | 0 |
| | ctrl. | | | | | | | | |
| 2 | 39A | 100 | 29 (0) | 34.7 (0) | * | 44.6 (0.2) | 9 | * | 465 |
| | | 50 | 36 (1) | 29.8 (0.7) | * | 39.3 (0.7) | 11 | * | 397 |
| | | 10 | 122 (3) | 10.8 (0.3) | 1694 (57) | 18.6 (0.2) | 37 | 84 | 135 |
| 2 | 43B1 | 100 | 238 (4) | 5.3 (0) | 2300 (32) | 10.8 (0.2) | 67 | 99 | 37 |
| | | 50 | 258 (5) | 5.2 (0.2) | 2301 (29) | 10.2 (0.2) | 72 | 99 | 29 |
| | | 10 | 317 (1) | 5 (0) | 2341 (34) | 8.6 (0.2) | 89 | 101 | 9 |
| 2 | 43D7 | 100 | 199 (6) | 5.1 (0.2) | 2124 (27) | 11.2 (0.2) | 56 | 91 | 42 |
| | | 50 | 234 (1) | 5 (0) | 2190 (15) | 10.3 (0) | 66 | 94 | 30 |
| | | 10 | 312 (3) | 5 (0) | 2343 (49) | 8.9 (0.2) | 88 | 101 | 13 |
| 2 | 43Ea | 100 | 308 (2) | 5 (0) | 2349 (9) | 9 (0) | 87 | 101 | 14 |
| | | 50 | 316 (3) | 5 (0) | 2430 (69) | 8.7 (0) | 89 | 105 | 10 |
| | | 10 | 337 (4) | 5 (0) | 2416 (82) | 8.3 (0) | 95 | 104 | 5 |
| 2 | 54E | 100 | 108 (3) | 12.2 (0.2) | 1589 (13) | 20.2 (0.2) | 30 | 68 | 156 |
| | | 50 | 191 (2) | 8 (0) | 2109 (51) | 14.3 (0) | 54 | 91 | 81 |
| | | 10 | 311 (5) | 5 (0) | 2275 (41) | 8.8 (0.2) | 87 | 98 | 11 |
| 2 | Isotype | 100 | 351 (2) | 4.7 (0) | 2304 (14) | 7.9 (0.2) | 99 | 99 | 0 |
| | | 50 | 353 (1) | 5 (0) | 2391 (29) | 8.2 (0.2) | 99 | 103 | 4 |
| | | 10 | 348 (1) | 5 (0) | 2367 (9) | 8.3 (0) | 98 | 102 | 5 |
| 2 | Plasma | NA | 356 (1) | 4.9 (0.2) | 2323 (76) | 8.11 (0.3) | 100 | 100 | 3 |
| | ctrl. | | | | | | | | |

* Groups with “No Tail Found” Errors when the software cannot calculate the ETP.

TABLE 45
Thrombin Generation Assay with 10 min Antibody Pre-Incubation

| | | | Peak | Lag | | | | | |
|---|---|---|---|---|---|---|---|---|---|
| | | Ab | IIa | Time | ETP | ttPeak | % | | |
| | | conc. | [nM] | [min] | [nM · min] | [min] | Peak | % | % |
| Plate | Sample | (nM) | (SD) | (SD) | (SD) | (SD) | IIa | ETP | ttPeak |
| 1 | 1F | 100 | 20 (1) | 29.5 (0.2) | * | 40.8 (0.6) | 7 | * | 483 |
| | | 50 | 23 (0) | 26.5 (0.7) | * | 37.3 (0.4) | 8 | * | 433 |
| | | 10 | 44 (2) | 13.8 (0.5) | 742 (23) | 22.4 (0.4) | 16 | 41 | 220 |
| 1 | 25A | 100 | 291 (3) | 3.3 (0.1) | 1964 (36) | 6.7 (0.1) | 106 | 108 | −4 |
| | | 50 | 290 (0) | 3.3 (0.1) | 1972 (22) | 6.8 (0) | 106 | 108 | −3 |
| | | 10 | 284 (1) | 3.3 (0.1) | 1899 (21) | 6.8 (0) | 104 | 104 | −3 |
| 1 | 25A3 | 100 | 290 (3) | 3.1 (0) | 1893 (28) | 6.4 (0) | 106 | 104 | −9 |
| | | 50 | 284 (4) | 3.1 (0) | 1875 (16) | 6.4 (0) | 104 | 103 | −9 |
| | | 10 | 288 (3) | 3.1 (0) | 1901 (26) | 6.4 (0) | 105 | 105 | −9 |
| 1 | 25G1 | 100 | 311 (3) | 3.1 (0) | 1954 (20) | 6.3 (0.1) | 114 | 107 | −10 |
| | | 50 | 311 (1) | 3.1 (0) | 1951 (22) | 6.1 (0) | 114 | 107 | −13 |
| | | 10 | 302 (3) | 3.1 (0) | 1877 (33) | 6.1 (0) | 110 | 103 | −13 |
| 1 | 29E | 100 | 76 (1) | 14.7 (0.1) | 1201 (24) | 24.3 (0.3) | 28 | 66 | 247 |
| | | 50 | 83 (1) | 14.1 (0) | 1300 (17) | 23.6 (0.1) | 30 | 72 | 237 |
| | | 10 | 98 (1) | 9.4 (0) | 1408 (11) | 18.1 (0) | 36 | 77 | 159 |
| 1 | Isotype | 100 | 288 (2) | 3.4 (0) | 1922 (28) | 6.8 (0) | 105 | 106 | −3 |
| | | 50 | 292 (2) | 3.4 (0) | 1921 (25) | 6.8 (0) | 107 | 106 | −3 |
| | | 10 | 290 (3) | 3.4 (0) | 1926 (38) | 6.8 (0) | 106 | 106 | −3 |
| 1 | TF-011 | 100 | 26 (0) | 23.8 (1.1) | * | 34.2 (0.9) | 9 | * | 389 |
| | | 50 | 27 (1) | 22.4 (0.1) | * | 33 (0.1) | 10 | * | 371 |
| | | 10 | 46 (3) | 13.5 (0.5) | 792 (55) | 22.5 (0.2) | 17 | 44 | 221 |
| 1 | 5G9 | 100 | 22 (0) | 26.7 (0.3) | * | 37.5 (0.5) | 8 | * | 436 |
| | | 50 | 23 (3) | 23.6 (2.2) | * | 34 (2.4) | 8 | * | 386 |
| | | 10 | 30 (1) | 19.3 (0.4) | * | 29 (0.8) | 11 | * | 314 |
| 1 | 10H10 | 100 | 169 (3) | 3.4 (0) | 1795 (36) | 9.3 (0.1) | 62 | 99 | 33 |
| | | 50 | 175 (4) | 3.4 (0) | 1754 (20) | 9.2 (0.1) | 64 | 96 | 31 |
| | | 10 | 235 (8) | 3.4 (0) | 1807 (42) | 7.8 (0) | 86 | 99 | 11 |
| 1 | Plasma | NA | 274 (1) | 3.4 (0) | 1818 (24) | 7 (0.1) | 100 | 100 | 0 |
| | ctrl. | | | | | | | | |
| 2 | 39A | 100 | 19 (1) | 33.6 (0.7) | * | 44.6 (0.9) | 7 | * | 537 |
| | | 50 | 22 (0) | 30.7 (0.1) | * | 41.4 (0.1) | 8 | * | 491 |
| | | 10 | 36 (1) | 19.6 (0.7) | * | 29.3 (0.8) | 13 | 0 | 319 |
| 2 | 43B1 | 100 | 167 (0) | 4 (0) | 1806 (15) | 9.8 (0.1) | 59 | 98 | 40 |
| | | 50 | 174 (1) | 3.8 (0.1) | 1831 (22) | 9.6 (0) | 62 | 99 | 37 |
| | | 10 | 222 (5) | 3.7 (0.1) | 1841 (37) | 8.3 (0) | 79 | 100 | 19 |
| 2 | 43D7 | 100 | 123 (2) | 4 (0) | 1673 (27) | 11.5 (0.1) | 44 | 91 | 64 |
| | | 50 | 122 (1) | 3.7 (0.1) | 1639 (29) | 11.3 (0) | 43 | 89 | 61 |
| | | 10 | 194 (5) | 4 (0) | 1796 (35) | 8.8 (0.1) | 69 | 97 | 26 |
| 2 | 43Ea | 100 | 244 (2) | 3.5 (0.1) | 1857 (42) | 7.5 (0.1) | 87 | 101 | 7 |
| | | 50 | 245 (0) | 3.6 (0) | 1851 (29) | 7.6 (0) | 87 | 100 | 9 |
| | | 10 | 262 (1) | 3.6 (0) | 1877 (15) | 7.3 (0) | 93 | 102 | 4 |
| 2 | 54E | 100 | 37 (1) | 22.3 (0.2) | * | 33 (0.5) | 13 | * | 371 |
| | | 50 | 44 (1) | 18.3 (0.4) | * | 28.2 (1) | 16 | * | 303 |
| | | 10 | 121 (4) | 6.5 (0.1) | 1523 (20) | 13.7 (0.3) | 43 | 83 | 96 |
| 2 | Isotype | 100 | 275 (2) | 3.6 (0) | 1862 (23) | 7.3 (0) | 98 | 101 | 4 |
| | | 50 | 284 (0) | 3.6 (0) | 1899 (15) | 7.2 (0.1) | 101 | 103 | 3 |
| | | 10 | 281 (3) | 3.6 (0) | 1877 (13) | 7.3 (0) | 100 | 102 | 4 |
| 2 | Plasma | NA | 282 (2) | 3.8 (0.1) | 1845 (22) | 7.3 (0) | 100 | 100 | 4 |
| | ctrl. | | | | | | | | |

* Groups with “No Tail Found” Errors when the software cannot calculate the ETP.

### Example 19: FXa Conversion Assay and FVIIa Competition Assay with Previously Described Anti-TF Antibodies

The previously described TF-specific antibodies TF-01 1, 5G9 and 10H10 (Breij et al., Cancer Res, 2014, 74:1214-1226; Versteeg et al., Blood, 2008, 111:190-199; each of which is incorporated by reference in its entirety) were tested in FXa conversion assay and FVIIa competition assay.

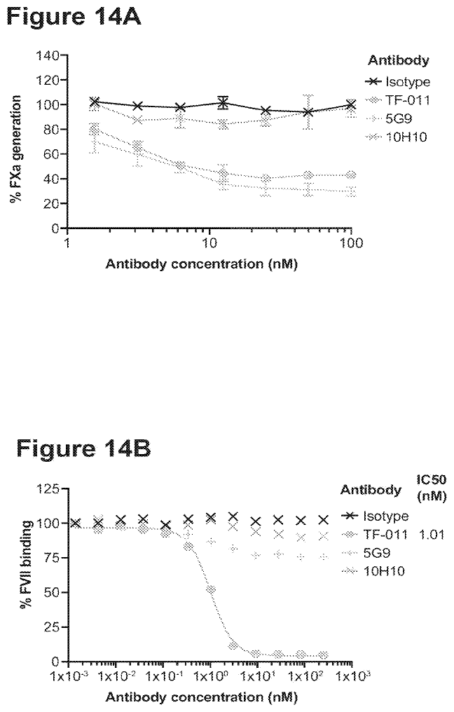

To evaluate the ability of TF:FVIIa to convert FX into FXa in the presence of human antibodies against TF, a cell-based FX conversion assay was conducted as described in Larsen et al., J Biol Chem, 2010, 285:19959-19966, which is incorporated by reference in its entirety. Briefly, 5×10⁴ MDA-MB-231 cells (ATCC, Manassas, VA, USA) were plated into tissue culture-treated black 96-well plates (Greiner Bio-One, Monroe, NC, USA) and cultured overnight. After removal of the cell culture media and addition of a final concentration of 200 nM of FX in a HEPES buffer with 1.5 mM CaCl₂), cells were incubated with a titration of the antibodies for 15 min at 37° C. Upon reconstitution of the binary TF:FVIIa complex with a final concentration of 20 nM of FVIIa, cells were incubated for 5 min at 37° C. After quenching the reaction with ethylenediaminetetraacetic acid (EDTA) in a black 94-well plate, generated FXa was measured with 50 μM of SN-7 6-amino-1-naphthalenesulfonamide-based fluorogenic substrate (Haematologic Technologies, Essex Junction, VT, USA) on an Envision plate reader equipped with an Umbelliferone 355 excitation filter, an Umbelliferone 460 emission filter, and a LANCE/DELFIA top mirror (Perkin Elmer, Waltham, MA, USA). FXa conversion percentages (% FXa) in the presence of an anti-TF antibody titration relative to a no antibody control are plotted in FIG. 14A.

To evaluate competition between FVIIa and the human antibodies against TF, TF-positive MDA-MB-231 cells (ATCC, Manassas, VA, USA) were first incubated for 1 hr on ice with a titration of the human antibodies against TF or an isotype control. Subsequently, FVII-Fc conjugated to Alexa488 was added to the antibody-cell mixture at a final concentration of 20 nM. After another 1 hr incubation on ice, cells were washed, stained with a viability dye, and analyzed by flow cytometry. The Alexa488 fluorescence data from viable cells was summarized using median fluorescence intensity (MFI). FVII-Fc binding was summarized with % FVII-Fc binding=[MFI_{antibody labeled cells}−MFI_{unstained cells}]/[MFI_{IgG1 control labeled cells}−MFI_{unstained cells}]. Percentage of FVIIa binding (% FVIIa) in the presence of an anti-TF antibody titration relative to an isotype control is plotted in FIG. 14B.

As presented in FIG. 14A, TF-011 and 5G9 inhibited FX conversion by 57-59% and 67-70% at concentrations of 25, 50, and 100 nM. 10H10 did not significantly inhibit FX conversion at these three concentrations.

As presented in FIG. 14B, TF-011 effectively competed with FVII, whereas 5G9 and 10H10 showed less than 25% and 10% competition at the highest concentration of antibody, respectively.

These results indicate that 5G9 predominantly competes with substrate FX binding, resulting in the observed inhibition of FX conversion and thrombin generation. TF-011 inhibits thrombin generation by competing with FVIIa for binding to TF. However, 10H10 inhibits TF-FVIIa mediated signaling without substantially affecting binding of FVIIa to TF. These findings are consistent with previous observations described in Huang et al., J Mol Biol, 1998, 275:873-894; Ruf et al., Biochem J, 1991, 278:729-733; and Teplyakov et al., Cell Signal, 2017, 36:139-144; each of which is incorporated by reference in its entirety.

### Example 20: Antibody Competition Assay

Alexa Fluor antibodies were generated using Alexa Fluor 488 5-sulfo-dichlorophenol esters (ThermoFisher Scientific) following manufacturer's protocol. Excess Alexa Fluor dye was removed from the antibody dye conjugate preparations by gel filtration (ThermoFisher Scientific).

To evaluate competition between a first human antibody against TF and 25A3, TF-positive MDA-MB-231 cells (ATCC, Manassas, VA, USA) were first incubated for 1 hr on ice with a titration of the first human antibody against TF. Subsequently, a final concentration of 20 nM of 25A3 conjugated to Alexa488 was added to the antibody cell mixture. After another 1 hr incubation on ice, cells were washed, stained with a viability dye, and analyzed by flow cytometry. The Alexa488 fluorescence data from viable cells was summarized using median fluorescence intensity. 25A3 binding was summarized with % 25A3 binding=[MFI_{antibody labeled cells}−MFI_{unstained cells}]/[MFI_{IgG1 control labeled cells}−MFI_{unstained cells}].

To evaluate competition between a first human antibody against TF and 43D7, TF-positive MDA-MB-231 cells (ATCC, Manassas, VA, USA) were first incubated for 1 hr on ice with a titration of the first human antibody against TF. Subsequently, a final concentration of 20 nM of 43D7 conjugated to Alexa488 was added to the antibody cell mixture. After another 1 hr incubation on ice, cells were washed, stained with a viability dye, and analyzed by flow cytometry. The Alexa488 fluorescence data from viable cells was summarized using median fluorescence intensity. 43D7 binding was summarized with % 43D7 binding=[MFI_{antibody labeled cells}−MFI_{unstained cells}]/[MFI_{IgG1 control labeled cells}−MFI_{unstained cells}].

To evaluate competition between a first human antibody against TF and 39A, TF-positive MDA-MB-231 cells (ATCC, Manassas, VA, USA) were first incubated for 1 hr on ice with a titration of the first human antibody against TF. Subsequently, a final concentration of 20 nM of 39A conjugated to Alexa488 was added to the antibody cell mixture. After another 1 hr incubation on ice, cells were washed, stained with a viability dye, and analyzed by flow cytometry. The Alexa488 fluorescence data from viable cells was summarized using median fluorescence intensity. 39A binding was summarized with % 39A binding=[MFI_{antibody labeled cells}−MFI_{unstained cells}]/[MFI_{IgG1 control labeled cells}−MFI_{unstained cells}].

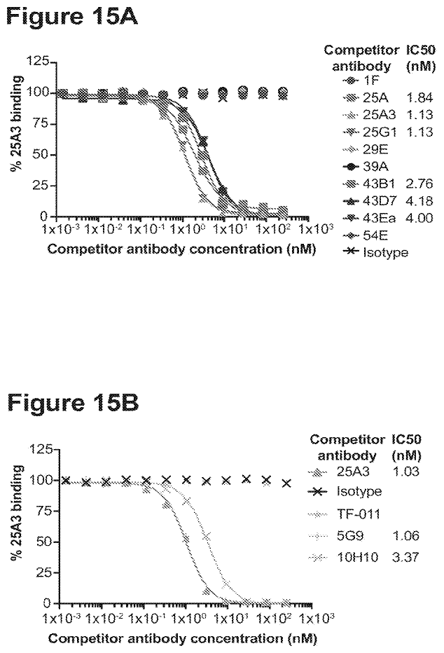

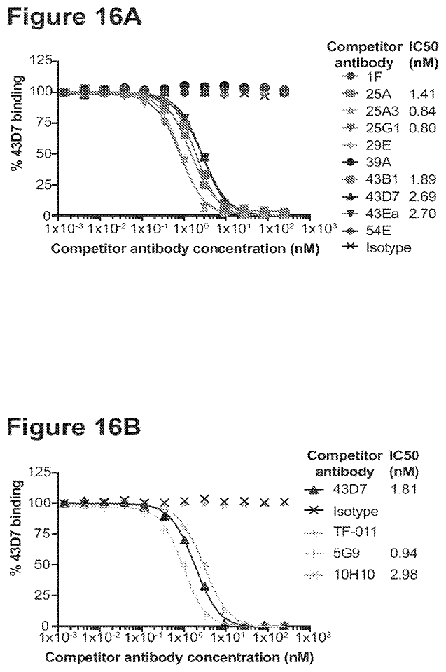

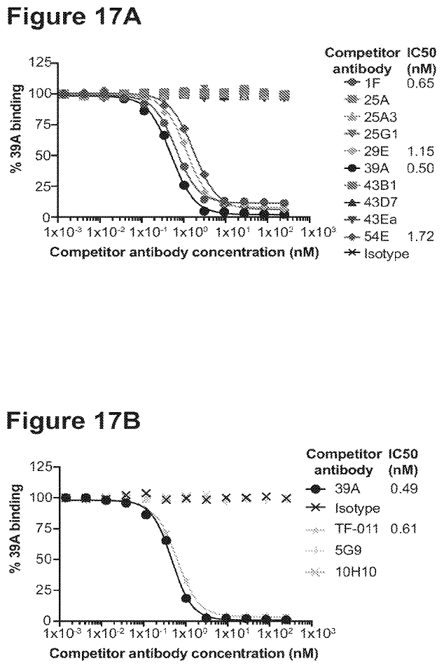

% 25A3 binding, % 43D7 binding, and % 39A binding are shown in FIGS. 15A and 15B, FIGS. 16A and 16B, and FIGS. 17A and 17B, respectively. Antibodies from groups 25 and 43, 5G9, and 10H10 reduced % 25A3 binding and % 43D7 binding and did not reduce % 39A binding. Antibodies from groups 1, 29, 39, and 54, and TF-011 reduced % 39A binding and did not reduce % 25A3 binding and % 43D7 binding.

While the antibody competition assay results indicate that groups 25 and 43 antibodies, 5G9, and 10H10 may bind to the same or an overlapping epitope of human TF or may affect the TF binding of each other through an allosteric mechanism, the chimeric TF construct mapping experiments as described elsewhere in this disclosure demonstrate that group 25 antibodies, group 43 antibodies, 5G9 and 10H10 bind distinct epitopes. In addition, while the antibody competition assay results indicate that antibodies of groups 1, 29, 39, and 54, and TF-011 may bind to the same or an overlapping epitope of human TF or may affect the TF binding of each other through an allosteric mechanism, the chimeric TF construct mapping experiments as described elsewhere in this disclosure demonstrate that the antibodies of groups 29, 39 and 54 bind epitopes distinct from TF-011's epitope.

### Example 21: Anti-TF Antibody Internalization

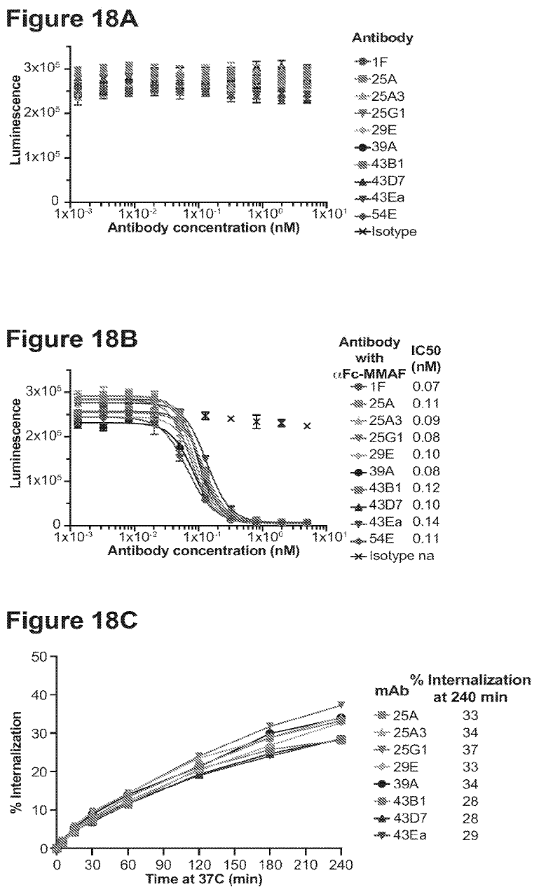

To evaluate internalization of the anti-TF antibodies, a cytotoxicity assay was conducted as described in Liao-Chan et al., PLoS One, 2015, 10:e0124708, which is incorporated by reference in its entirety. Briefly, cells were plated in 384-well plates (Greiner Bio-One, Monroe, NC, USA) at 4×10³ cells per well in 40 μl of media. Antibodies and an anti-human Fc Fab conjugated to the tubulin inhibitor mono-methyl auristatin F (MMAF) (Moradec, San Diego, CA, USA) were serially diluted starting at 5 and 30 nM, respectively. The anti-human Fc Fab conjugated to MMAF consisted of a polyclonal antibody specific to the Fc region of human IgGs with a DAR of 1.2 to 1.5. Plates were incubated for 3 days, followed by lysis in CellTiter-Glo (CTG) assay reagent (Promega, Madison, WI, USA). CTG luminescence was measured on an Envision plate reader and the mean and standard deviation of 4 replicates graphed in Prism (GraphPad, La Jolla, CA, USA). For each anti-TF antibody, the IC₅₀ and its associated 95% confidence interval were calculated in Prism using a 4-parameter binding model. The cell viability results after incubation with anti-TF antibodies and anti-TF antibody Fab:MMAF complexes are shown in FIGS. 18A and 18B. The 95% confidence intervals for the IC₅₀ values are shown in Table 46.

Internalization of the anti-TF antibodies was also evaluated by a quantitative assay based on internalized fluorescence and quenched surface-fluorescence. Cell surface fluorescence quenching was assessed as described in Liao-Chan et al., PLoS One, 2015, 10:e0124708. Briefly, 1.2×10⁵ MDA-MB-231 cells were pre-incubated with 100 nM of A488-conjugated antibodies in media for 2 hr on ice. After 2 washes, cells were resuspended in cold media and pulsed for up to 4 hr at 37° C. Cells were rapidly chilled and incubated with or without 300 nM of anti-A488 antibody (clone 19A) for 30 min on ice. After 2 washes, dead cells were labeled with DAPI and samples were analyzed on a Novocyte flow cytometer (ACEA Biosciences). The median fluorescence intensities (MFIs) at each anti-A488 mAb concentration were normalized against the isotype control to obtain a normalized MFI percentage. Internalized fluorescence was calculated from quenched and non-quenched sample data by correcting for incomplete surface quenching: 1−(N₁−Q₁)/(N₁−(N₁Q₀/N₀)) with N₁=unquenched MFI at each time point (t₁); Q₁=Quenched MFI at t₁; Q₀=Quenched MFI for the sample kept on ice (t₀); N₀=Unquenched MFI at t₀. Percent internalization of anti-TF antibodies conjugated to A488 is shown in FIG. 18C.

Because Fab:MMAF binds the Fc region of the TF-specific antibodies, cellular uptake of these complexes can trigger cell death. While the TF-specific antibodies alone had no impact on cell viability in three-day cultures of TF-positive A431 cells (FIG. 18A), the TF-specific antibodies in complex with Fab:MMAF showed dose-dependent cell killing with IC₅₀ values ranging between 0.07 and 0.14 nM (FIG. 18B).

Cellular uptake was corroborated with fluorescently labeled TF-specific antibodies. In a quantitative assay based on internalized fluorescence and quenched surface-fluorescence, the TF-specific antibodies showed between 28 and 37% internalization after a 4 h incubation (FIG. 18C).

These results indicate that the tested anti-TF antibodies can medicate internalization and toxin delivery into TF-positive cells.

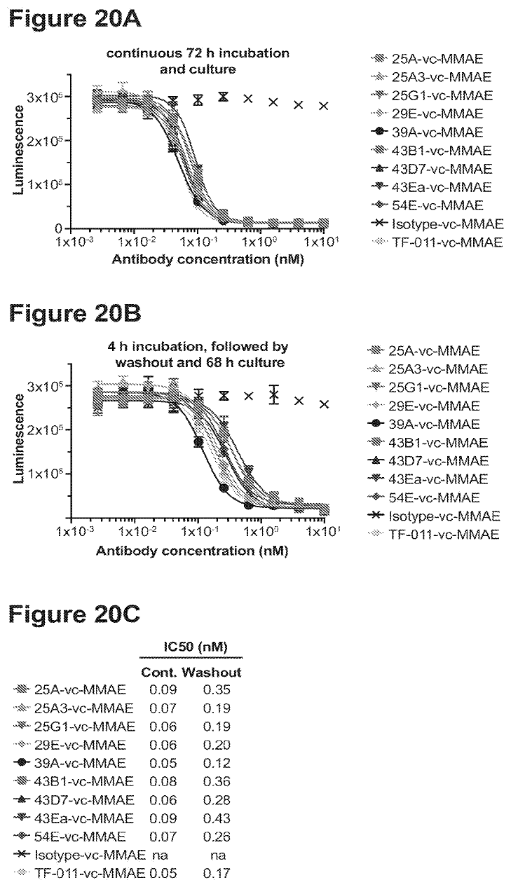

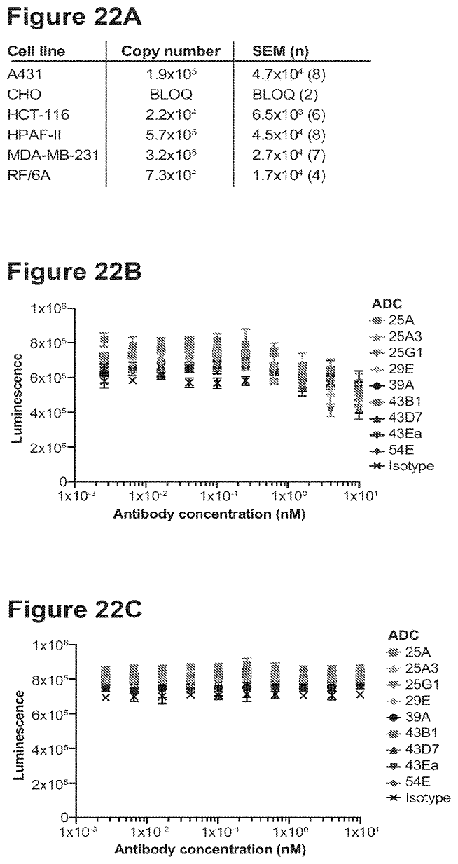

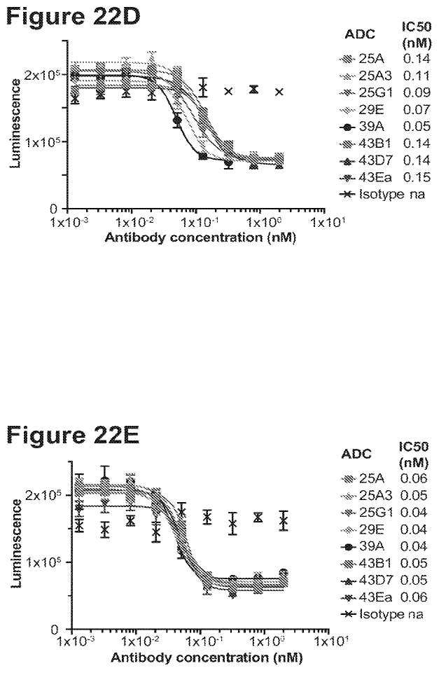

TABLE 46
ADC Data With Ranking (Continuous Incubation)

| | | Cell line: | | | | | | | | | | | | |
|---|---|---|---|---|---|---|---|---|---|---|---|---|---|---|
| | | A431 | | | A431 | | | MDA-MB-231 | | | HPAF-II | | | |
| | | ADC format: | | | | | | | | | | | | |
| | | Secondary ADC | | | Primary ADC | | | Primary ADC | | | Primary ADC | | | |
| | | Treatment: | | | | | | | | | | | | |
| | | Continuous | | | Continuous | | | Continuous | | | Continuous | | | |
| | | FIG.: | | | | | | | | | | | | |
| | | FIG. 18B | | | FIG. 20A | | | FIG. 22D | | | FIG. 22E | | | Continuous |
| | | Binding data: | | | | | | | | | | | | Primary |
| | | IC₅₀ | | | IC₅₀ | | | IC₅₀ | | | IC₅₀ | | | ADC |
| | | (nM) | 95% CI | rank | (nM) | 95% CI | rank | (nM) | 95% CI | rank | (nM) | 95% CI | rank | RANK |
| Anti- | 1F | 0.07 | 0.06 to 0.07 | Not | Not tested | | | Not tested | | | Not tested | | | Not |
| body: | | | | in- | | | | | | | | | | included |
| | | | | cluded | | | | | | | | | | |
| | 25A | 0.11 | 0.10 to 0.11 | 6 | 0.09 | 0.08 to 0.09 | 7 | 0.14 | 0.12 to 0.16 | 7 | 0.06 | 0.05 to 0.07 | 8 | 7 |
| | 25A3 | 0.09 | 0.08 to 0.09 | 3 | 0.07 | 0.07 to 0.08 | 5 | 0.11 | 0.10 to 0.12 | 4 | 0.05 | 0.04 to 0.05 | 5 | 4 |
| | 25G1 | 0.08 | 0.07 to 0.08 | 1 | 0.06 | 0.06 to 0.06 | 3 | 0.09 | 0.08 to 0.10 | 3 | 0.04 | 0.04 to 0.05 | 3 | 3 |
| | 29E | 0.10 | 0.09 to 0.10 | 4 | 0.06 | 0.05 to 0.06 | 2 | 0.07 | 0.07 to 0.08 | 2 | 0.04 | 0.04 to 0.05 | 2 | 2 |
| | 39A | 0.08 | 0.08 to 0.09 | 2 | 0.05 | 0.05 to 0.05 | 1 | 0.05 | 0.05 to 0.05 | 1 | 0.04 | 0.03 to 0.05 | 1 | 1 |
| | 43B1 | 0.12 | 0.11 to 0.13 | 7 | 0.08 | 0.08 to 0.08 | 6 | 0.14 | 0.13 to 0.15 | 5 | 0.05 | 0.04 to 0.06 | 4 | 5 |
| | 43D7 | 0.10 | 0.10 to 0.10 | 5 | 0.06 | 0.06 to 0.07 | 4 | 0.14 | 0.12 to 0.16 | 6 | 0.05 | 0.05 to 0.06 | 6 | 6 |
| | 43Ea | 0.13 | 0.13 to 0.14 | 8 | 0.09 | 0.09 to 0.10 | 8 | 0.15 | 0.13 to 0.17 | 8 | 0.06 | 0.05 to 0.06 | 7 | 8 |

| | | | | | | | | | |
|---|---|---|---|---|---|---|---|---|---|
| 54E | 0.11 | 0.11 to 0.12 | Not | 0.07 | 0.07 to 0.07 | Not | Not tested | Not tested | Not |
| | | | in- | | | in- | | | included |
| | | | cluded | | | cluded | | | |

| Isotype | Not applicable | Not applicable | | Not applicable | Not applicable | Not |
|---|---|---|---|---|---|---|
| | | | | | | included |
| TF-011 | Not tested | 0.05 | 0.05 to 0.05 | Not tested | Not tested | Not |
| | | | | | | included |

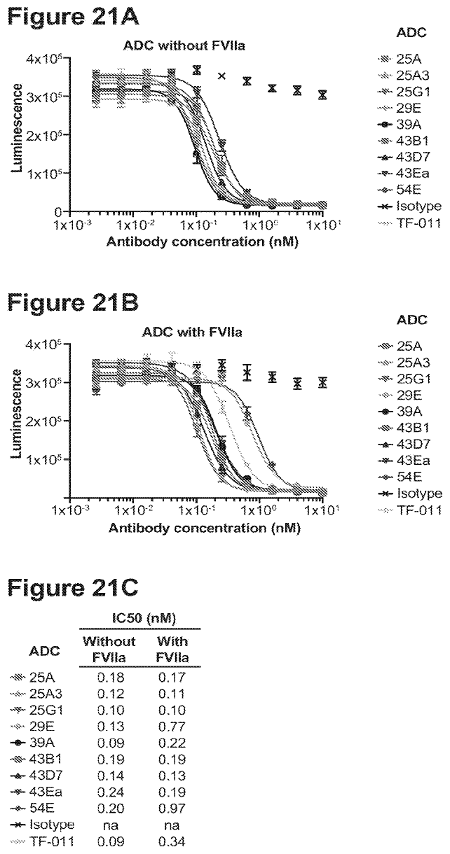

TABLE 47
ADC Data With Ranking (4 h Incubation)

| | | Cell line: | | | | | | |
|---|---|---|---|---|---|---|---|---|
| | | A431 | | | A431 | | | |
| | | ADC format: | | | | | | |
| | | Primary ADC | | | Primary ADC | | | |
| | | Treatment: | | | | | | |
| | | 4 hr, followed by washout | | | 4 hr, followed by washout | | | |
| | | FIG.: | | | | | | |
| | | FIG. 20B | | | FIG. 21A | | | |
| | | Measurement: | | | | | | 4 hr Primary |
| | | IC₅₀ (nM) | 95% CI | rank | IC₅₀ (nM) | 95% CI | rank | ADC RANK |
| Antibody: | 1F | Not tested | | | Not tested | | | Not included |
| | 25A | 0.35 | 0.32 to 0.39 | 6 | 0.18 | 0.17 to 0.19 | 6 | 6 |
| | 25A3 | 0.19 | 0.17 to 0.21 | 3 | 0.12 | 0.11 to 0.12 | 3 | 3 |
| | 25G1 | 0.19 | 0.17 to 0.20 | 2 | 0.10 | 0.09 to 0.10 | 2 | 2 |
| | 29E | 0.20 | 0.18 to 0.21 | 4 | 0.13 | 0.12 to 0.14 | 4 | 4 |
| | 39A | 0.12 | 0.11 to 0.13 | 1 | 0.09 | 0.09 to 0.10 | 1 | 1 |
| | 43B1 | 0.36 | 0.32 to 0.41 | 7 | 0.19 | 0.17 to 0.20 | 7 | 7 |
| | 43D7 | 0.28 | 0.25 to 0.30 | 5 | 0.14 | 0.13 to 0.15 | 5 | 5 |
| | 43Ea | 0.43 | 0.39 to 0.48 | 8 | 0.24 | 0.22 to 0.25 | 8 | 8 |
| | 54E | 0.26 | 0.24 to 0.29 | | 0.20 | 0.18 to 0.22 | | Not included |
| | Isotype | Not applicable | | | Not applicable | | | Not included |
| | TF-011 | 0.17 | 0.16 to 0.18 | | 0.09 | 0.09 to 0.10 | | Not included |

### Example 22: Cell-Based Binding Assay of Antibody-Drug Conjugates (ADCs)

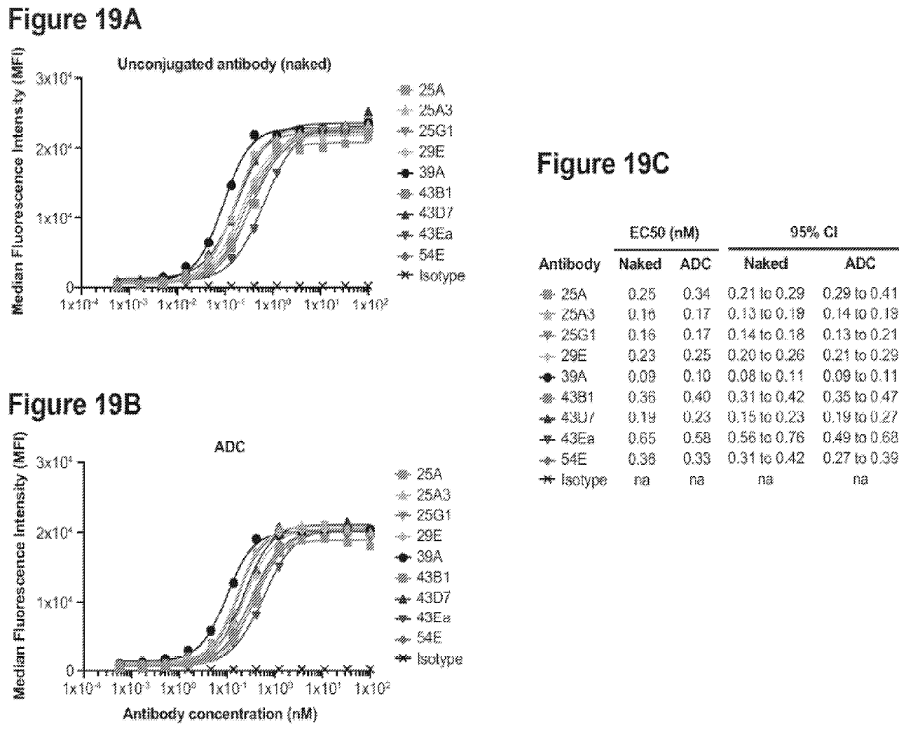

To evaluate the cell binding properties of ADCs, binding of anti-TF antibodies and anti-TF ADCs to endogenous human TF expressing HCT116 cells was was assessed as previously described in Liao-Chan et al., PLoS One, 2015, 10:e0124708, which is incorporated by reference in its entirety. Briefly, 1.2×10⁵ cells collected with Cellstripper (Mediatech, Manassas, VA, USA) were incubated with a twelve-point 1:3 dilution titration of anti-human TF antibody or ADC starting at 100 nM for 2 hr on ice. After 2 washes, cells labeled with antibody or ADC were incubated for 30 min on ice with 150 nM of Goat Phycoerythrin (PE) F(ab′)₂ fragment goat anti-human IgG, Fcγ fragment specific (Jackson ImmunoResearch, West Grove, PA, USA) or FITC-labeled F(ab′)₂ fragment goat anti-human kappa (SouthernBiotech, Birmingham, AL, USA), respectively. After 2 washes, dead cells were labeled with TO-PRO-3 Iodide (ThermoFisher Scientific) and samples were analyzed on a CytoFLEX flow cytometer (Beckman Coulter, Brea, CA, USA) or Novocyte flow cytometer (ACEA Biosciences, San Diego, CA, USA). The median fluorescence intensities (MFIs) at each dilution were plotted and cell EC₅₀'s were derived using a 4-parameter binding model in Prism (GraphPad, La Jolla, CA, USA). FIGS. 19A and 19B exhibit the binding curves of anti-TF antibodies and anti-TF ADCs, respectively. FIG. 19C lists the reportable cell EC₅₀'s and their 95% confidence intervals of the anti-TF antibodies and ADCs.

As shown in FIGS. 19A, 19B, and 19C, the cell binding properties of TF-specific ADCs are comparable to the cell binding properties of TF-specific antibodies, which indicates that the conjugation process of ADC did not alter the cell-binding properties of the TF-specific antibody moiety of the ADC.

### Example 23: Cytotoxicity Assays of Antibody-Drug Conjugates (ADCs)

To evaluate ADC cytotoxicity, A431cells were plated in 384-well plates (Greiner Bio-One). Anti-TF antibodies conjugated to MC-vc-PAB-MMAE were serially diluted as shown. The TF-specific ADCs were added to A431 cells, with either a 72 h incubation or a 4 h incubation followed by removal of excess ADC and culture for another 68 h. A431cells were lysed in CTG assay reagent after treatment. CTG luminescence was measured and the mean and standard deviation of 4 replicates graphed in Prism. For each ADC, the IC₅₀ and its associated 95% confidence interval were calculated in Prism using a 4-parameter binding model.

FIG. 20A shows the cell viability after titrations of anti-TF ADCs with a continuous 72 h incubation. FIG. 20B shows the cell viability after titrations of anti-TF ADCs with a 4 h incubation followed by removal of excess ADC and culture for another 68 h. FIG. 20C shows the reportable IC₅₀ values of ADCs under both the continuous treatment and the pulse treatment. The 95% confidence intervals for the IC₅₀'s of the continuous treatment and the pulse treatment are listed in Table 46 and Table 47 respectively.

Both treatments resulted in efficacious cell killing, with a 2.4 to 4.7-fold increase in IC₅₀ when excess ADC was removed from the culture after the 4 h incubation compared to the 72 h incubation. Removal of excess 25A3 and 39A ADC had the smallest impact on IC₅₀, with a 2.7 and 2.4-fold increase from 0.07 and 0.05 nM, respectively.

These results indicate that similar to the TF-specific antibodies, the TF-specific ADCs undergo substantial cellular internalization.

### Example 24: Cytotoxicity Assays in the Presence of FVIIa

To understand whether FVIIa interfered with the activity of the TF-specific ADC, we treated A431 cells for 4 h with the TF-specific ADCs (anti-TF antibodies conjugated to MC-vc-PAB-MMAE) in the absence or presence of FVIIa and measured cell viability 68 h later. A431 cells were pre-incubated for 30 min without or with 50 nM of FVIIa prior to the addition of an anti-TF ADC titration. Cell viability was determined by CTG assay. The mean and standard deviation of 4 replicates were graphed in Prism. For each ADC, the IC₅₀ were calculated in Prism using a 4-parameter binding model.

The cell viability after titrations of anti-TF ADCs in the absence or presence of FVIIa is shown in FIGS. 21A and 21B respectively. The reportable IC₅₀ values of ADCs in the absence or presence of FVIIa are listed in FIG. 21C.

While the ADCs that competed with FVIIa (29E, 39A, 54E and TF-011) were negatively affected by the presence of FVIIa by at least 2.3-fold, the ADCs that did not compete with FVIIa (group 25 and 43 antibodies) were equally efficacious in the absence or presence of FVIIa.

These results indicate that FVIIa does not interfere with the activity of anti-TF ADCs from groups 25 and 43.

### Example 25: Cytotoxicity Assays on Additional Cancer Cell Lines

To evaluate TF copy number on the cell surface of different cell lines, 1.2×10⁵ cells were harvested and incubated with 133 nM of anti-human TF antibody 5G9 on a mouse IgG2a backbone for 2 hr on ice. After 2 washes, QIFIKIT beads (Agilent) and cells labeled with anti-TF antibody were incubated for 30 min on ice with 150 nM of Goat Phycoerythrin (PE) F(ab′)₂ fragment goat anti-mouse IgG, Fc-gamma fragment specific (Jackson ImmunoResearch). After 2 washes, dead cells were labeled with TO-PRO-3 Iodide (ThermoFisher Scientific) and samples were analyzed on a CytoFLEX flow cytometer (Beckman Coulter). After gating for single live cells, the MFI's were determined using FlowJo (Flowjo, Ashland, OR, USA). A standard curve using QIFIKIT beads was generated in Prism using a 5-parameter binding model to determine copy number. The lower limit of quantitation was 1.9×10³ antibody binding sites (also referred to as copy number) and the upper limit of quantitation was 8.0×10⁵ antibody binding sites.

The TF copy number on A431, CHO, HCT-116, HPAF-II, MDA-MB-231, and RF/6A is listed in FIG. 22A. The level of surface TF ranged from 1.9×10⁵ to 5.7×10⁵ copies in A431, MDA-MB-231 and HPAF-II cells. HCT-116 cells expressed 2.2×10⁴ copies of surface TF and TF expression in CHO cells was below limit of quantitation (BLOQ). As 5G9 cross-reacts with Macaca fascicularis TF and the TF protein sequence between M. fascicularis and mulatta is identical, the level of surface TF in the M. mulatta cell line RF/6A was also qualified (1.7×10⁴ copies).

Cell viability of HCT-116, CHO, MDA-MB-231, and HPAF-II cells in the presence of titrations of anti-TF MMAE ADCs was shown in FIGS. 22B, 22C, 22D, and 22E respectively.

The TF-specific ADCs effectively reduced the viability of MDA-MB-231 and HPAF-II cancer cell lines (FIGS. 22D and 22E). Compared to the activity on MDA-MB-231 and HPAF-II cells, the ADCs were less efficacious on HCT-116 cells, with some activity at the highest concentration and no reportable IC₅₀ value (FIG. 22B). The TF-specific ADCs did not affect the viability of the CHO cultures (FIG. 22C).

These results indicate that the cytotoxicity of anti-TF ADCs is specific for TF positive cells.

When ranking the cell-killing efficacy of the ADCs on A431, HPAF-II and MDA-MB-231 cells, the top four ADCs in descending order were 39A, 29E, 25G1 and 25A3 (Table 46). When A431 cells were incubated for 4 h with the TF-specific ADCs followed by a washout, the top four ADCs in descending order were 39A, 25G1, 25A3 and 29E (Table 47). Thus, the top 2 ranking ADCs with no impact on coagulation were 25G1 and 25A3.

### Example 26: Intracellular Microtubule Network in the Presence of Antibody-Drug Conjugates (ADCs)

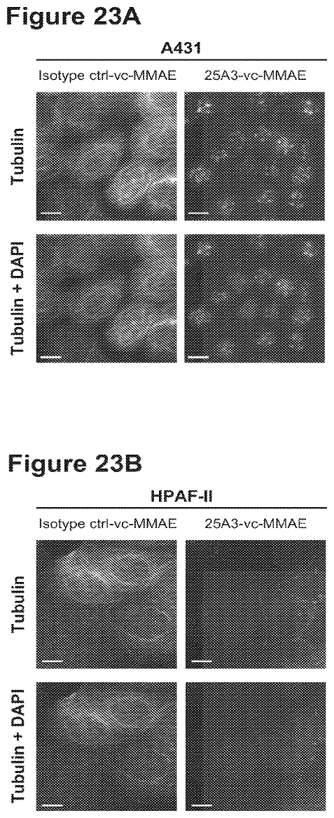

Immunofluorescence of the intracellular microtubule network of cells was conducted to illustrate the mechanism of action of the ADC. See Theunissen et al., Methods Enzymol, 2006, 409:251-284. Briefly, A431 or HPAF-II cells were seeded onto 8-well poly-D-lysine treated slides (Corning Inc, Corning, NY, USA). One day later, the culture medium was replaced with medium containing ADC at 5 nM. After twenty hours of ADC exposure, the cells were fixed for 15 min at room temperature with 4% paraformaldehyde (ThermoFisher Scientific). After three washes with PBS, the cells were permeabilized for 1 h with PBS containing 0.3% Triton X-100 and 5% normal goat serum. Next, the microtubule networks were stained for 3 h with anti-tubulin (11H10) rabbit mAb (Alexa Fluor 488 conjugate) (Cell Signaling Technology, Danvers, MA, USA) in PBS containing 1% BSA and 0.3% Triton X-100. After three washes, ProLong Gold Antifade reagent with DAPI (ThermoFisher Scientific) was added to the cells and the slide was mounted for microscopy by using a 0.17 mm coverslip. Image acquisition was conducted on a DMi8 fluorescence microscope (Leica Microsystems, Buffalo Grove, IL, USA) equipped with a sCMOS camera. The Leica LAS X software was used to acquire a system-optimized Z-stack of 6 to 7 microns. A sharp two-dimensional image from this Z-stack was created automatically with the extended depth of field (EDF) image feature. Representative images of tubulin staining of A431 or HPAF-II cells are shown in FIGS. 23A and 23B respectively.

While the isotype control ADC did not affect the microtubule network, the 25A3 ADC disrupted the microtubule network effectively in both A431 and HPAF-II cells.

These results indicate the MMAE-based anti-TF ADCs induce cytotoxicity in TF-positive cancer cells through disruption of the intracellular microtubule network.

### Example 27: Cytotoxicity Assays and G₂/M Arrest in HUVECs

To evaluate TF copy number on the cell surface of human umbilical vein endothelial cells (HUVECs), 1.2×10⁵ HUVECs were harvested and incubated with 133 nM of anti-human TF antibody 5G9 on a mouse IgG2a backbone for 2 hr on ice. After 2 washes, QIFIKIT beads (Agilent) and cells labeled with anti-TF antibody were incubated for 30 min on ice with 150 nM of Goat Phycoerythrin (PE) F(ab′)₂ fragment goat anti-mouse IgG, Fc-gamma fragment specific (Jackson ImmunoResearch). After 2 washes, dead cells were labeled with TO-PRO-3 Iodide (ThermoFisher Scientific) and samples were analyzed on a CytoFLEX flow cytometer (Beckman Coulter). After gating for single live cells, the MFI's were determined using FlowJo (Flowjo, Ashland, OR, USA). A standard curve using QIFIKIT beads was generated in Prism using a 5-parameter binding model to determine copy number. The lower limit of quantitation was 1.9×10³ antibody binding sites (also referred to as copy number) and the upper limit of quantitation was 8.0×10⁵ antibody binding sites.

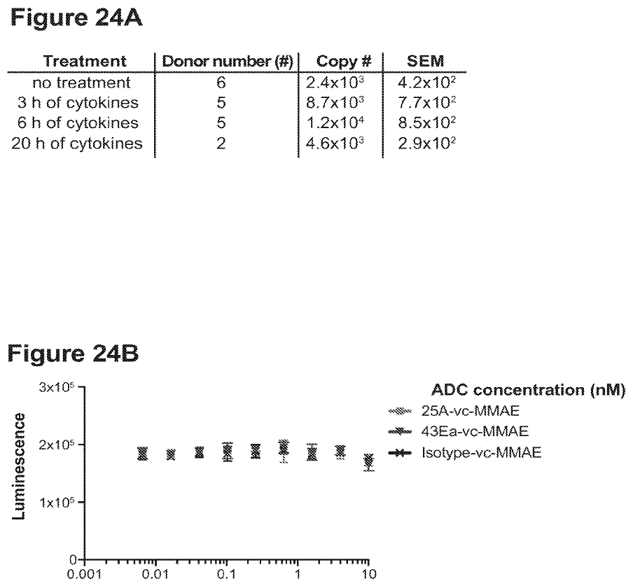

In response to injury, inflammatory and angiogenic factors transiently increase expression of surface TF in the vasculature. See Holy et al., Adv Pharmacol, 2010, 59:259-592, which is incorporated by reference in its entirety. The transient upregulation of TF in cell culture was mimicked by treating HUVECs with a combination of inflammatory cytokines (5 ng/mL IL1-beta, 25 ng/mL TNF-alpha and 50 ng/mL VEGF). As shown in FIG. 24A, surface TF levels increased from 2.4×10³ copies in the absence of inflammatory cytokines to 1.2×10⁴ copies after 6 h of cytokine treatment. The surface TF was ˜3-fold lower after 20 h of cytokine treatment relative to 6 h of treatment, which indicates that the cytokine-induced TF upregulation was transient.

For the ADC cytotoxicity assay, HUVEC cultures were seeded on half-area 96-well plates. The next day, the combination of inflammatory cytokines and a titration of ADCs was added to the cultures. Four days later viability of the cultures was assessed by lysis in CellTiter-Glo (CTG) assay reagent. As shown in FIG. 24B, the cell viability of inflammatory cytokine-treated HUVEC cultures was unaffected by the anti-TF ADCs, 25A-vc-MMAE and 43Ea-vc-MMAE. The results indicates that the inflammatory cytokine-treated endothelial cells are resistant to anti-TF ADCs.

To further understand the resistance of endothelial cells to anti-TF ADCs, cell cycle progression was evaluated 24 h after addition of the cytokines and TF-specific ADCs. Arrest at the the G₂/M phase of the cell cycle was analyzed as previously described in Theunissen et al, Methods Enzymol, 2006, 409:251-284. Briefly, low-passage HUVECs (Lifeline Cell Technologies, Frederick, MD, USA), propagated in VascuLife VEGF-Mv Endothelial media (Lifeline Cell Technologies), and HCT-116 cells were seeded on 12-well plates. The next day, media was removed and replaced with fresh media (no cytokines) or media containing 5 ng/mL IL1-beta, 25 ng/mL TNF-alpha and 50 ng/mL VEGF (with cytokines). A titration of MMAE-linked ADCs or free MMAE was added to the cells. After 24 h of treatment, cells were fixed in ice-cold 70% ethanol. Subsequently, the cells were washed with flow cytometry buffer (PBS, 1% FBS, 0.1% Triton) and stained for 1 h with a 1:100 dilution of phospho-Histone H3 (Ser10) (D2C8 PE Conjugate, Cell Signaling Technology). After 2 washes, the cells were treated for 20 min with 100 μg/mL PureLink RNAse A (ThermoFisher Scientific), followed by the addition of the viability dye TO-PRO-3 Iodide (ThermoFisher Scientific). 40,000 events were collected on a Novocyte flow cytometer. In the Flowjo data analysis software cell doublets and aneuploid cells were excluded. The pH3 signal was plotted against DNA content to determine the percentage of pH3-positive cells.

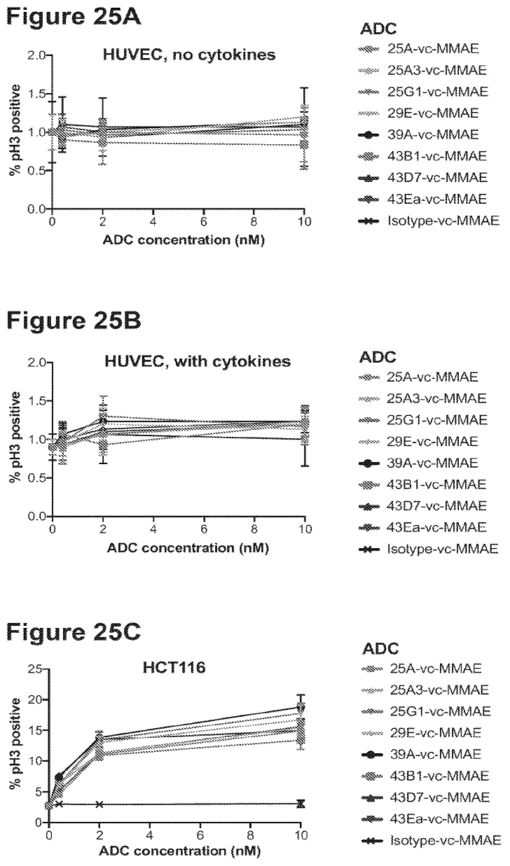

The percentage of pH3-positive cells (% pH3) with titrations of anti-TF ADCs on HUVECs in the absence or presence of inflammatory cytokines is shown in FIGS. 25A and 25B respectively. The percentage of pH3-positive cells (% pH3) with titrations of anti-TF ADCs on HCT-116 cells is shown in FIG. 25C.

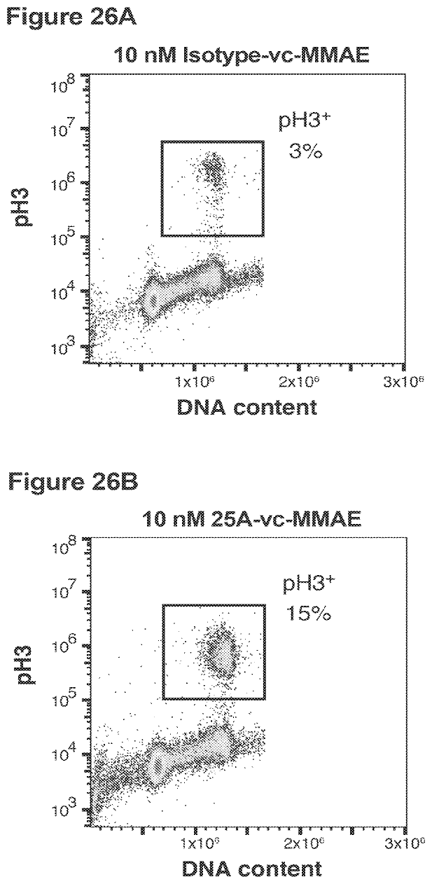

While the TF-specific ADCs induced an arrest at the G2/M phase of the cell cycle in HCT-116 cells, the ADCs did not impact cell cycle progression in HUVECs with or without inflammatory cytokine treatment. As shown in FIGS. 26A and 26B, the percentage of pH3-positive HCT-116 cells increased 5 times after treatment of 25A-vc-MMAE as compared to treatment of Isotype-vc-MMAE.

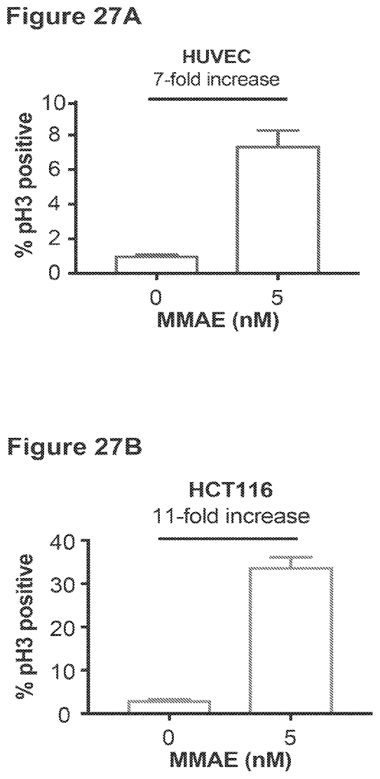

FIGS. 27A and 27B show that unconjugated MMAE increase the phosphorylation of histone H3 to a similar extent in both HCT-116 cells and HUVECs, indicating that the resistance in endothelial cells is specific for the MMAE-based ADC.

Taken together, these results indicate that the anti-TF ADCs do not affect the viability of HUVECs in the absence or presence of inflammatory cytokines.

### Example 28: Erk Phosphorylation Assay

For assessment of Erk phosphorylation, A431 cells were plated in 6-well plates (Corning) in media overnight. The following day, cells were washed once and serum starved in serum-free media. After starvation, cells were preincubated with 100 nM of anti-TF antibodies for 30 min at 37° C. FVIIa was spiked into the wells at 50 nM and incubated for 20 min at 37° C. for p-ERK induction. After induction, cells were lysed with RIPA Lysis and Extraction Buffer with Halt™ Protease and Phosphatase Inhibitor Cocktail (ThermoFisher Scientific). Western blot was performed with 20 μg of cell lysate using Phospho-p44/42 MAPK (Erk1/2) (Thr202/Tyr204) and p44/42 MAPK (Erk1/2) (137F5) (Cell Signaling Technology) as primary antibodies and Peroxidase AffiniPure Donkey Anti-Rabbit IgG (H+L) (Jackson ImmunoResearch) as a secondary antibody. Non-saturating band intensities for pErk and Erk were measured on an Amersham AI600 (GE Healthcare). Each pErk intensity was normalized against its respective Erk intensity and the no-antibody no-FVIIa sample intensity.

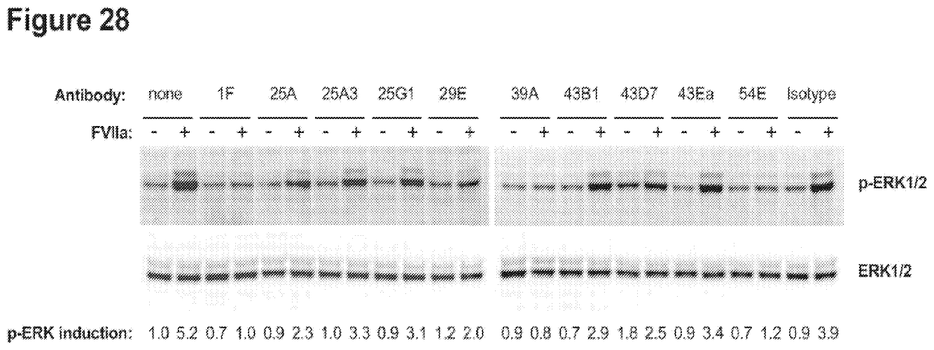

The Western blot results of pErk and Erk are shown in FIG. 28. Treatment with FVIIa induced Erk phosphorylation by 5.2 fold in cell cultures without pretreatment of anti-TF antibodies. The inducation of Erk phosphorylation was ablated by pretreatment with 1F, 39A and 54E (fold induction between 0.8 and 1.2) and attenuated by 29E and the members of groups 25 and 43 (fold induction between 2.0 and 3.4).

This data indicates that anti-TF antibodies inhibit FVIIa-dependent TF signaling when assessing Erk phosphorylation.

### Example 29: Antibody-Dependent Cellular Cytotoxicity (ADCC) Assay

To evaluate ADCC activity, an ADCC Reporter Bioassay Core Kit (Promega) was used following the manufacturer's protocol. Briefly, A431 cells were plated on a microtiter plate (Corning). The following day, the cells were incubated with a ten-point 1:3 dilution titration of anti-TF antibodies or the ADCs starting at 50 nM. An ADCC effector-to-target cell ratio of 8:1 was added to each well and incubated for 6 h at 37° C. Bio-Glo™ Luciferase Assay Reagent was added to each well to measure luminescence on an Envision plate reader (PerkinElmer, Waltham, MA, USA). The mean and standard deviation of 4 replicates were graphed in Prism. For each antibody and ADC, the EC₅₀ and its associated 95% confidence interval were calculated in Prism using a 4-parameter binding model.

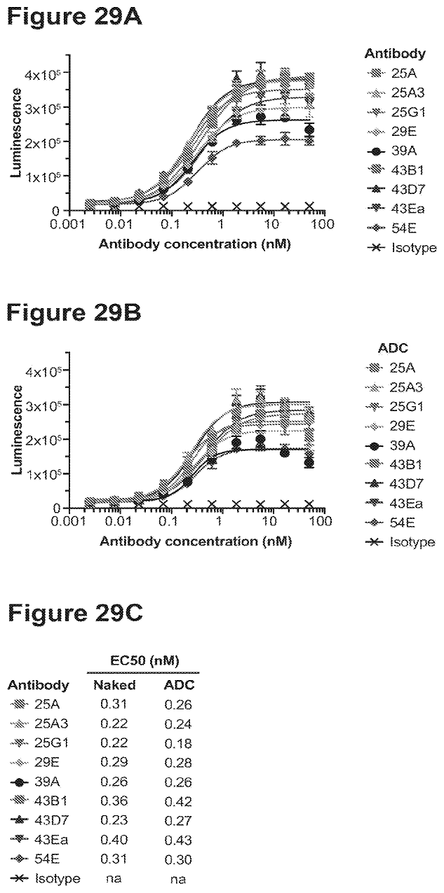

ADCC reporter luminescence after incubation with the reporter Jurkat cell line in the represece titrations of anti-TF antibodies or anti-TF ADCs is shown in FIGS. 29A and 29B respectively. The ADCC reporter luminescence EC₅₀ values for each anti-TF antibody or ADC are listed in FIG. 29C.

All the tested TF-specific antibodies and ADCs exerted induction of luciferase-dependent luminescence with EC₅₀ values ranging between 0.18 and 0.43 nM.

These data indicate that both the TF-specific antibodies and ADCs can induce antibody-dependent cellular cytotoxicity (ADCC) via the IgG1 Fc domain of the antibody.

### Example 30: Studies in Cell Line-Derived Xenograft (CDX) Models

To evaluate the efficacy of the ADCs in vivo, xenograft studies in immune compromised mice were performed as described in Kim et al., Blood Cancer J, 2015, 5:e316, which is incorporated by reference in its entirety. Briefly, the A431 epidermoid carcinoma and the HPAF-II pancreatic carcinoma cell lines were implanted subcutaneously in the flank of athymic nude mice (Charles River Laboratories, Wilmington, MA). Animals were randomized and treated as indicated in the figures. Body weight and tumor size assessments were performed bi-weekly. Animals were removed from study and euthanized once tumor size reached 1200 mm³ or skin ulceration was evident. In addition, the MTV curve for the treatment group in question was no longer shown once an animal was removed from study due to size. The animals' care was in accordance with institutional guidelines. Mean tumor volume (MTV) with the standard error of the mean (SEM) was plotted over time. Treatment efficacy was determined by calculating tumor growth inhibition (% TGI=100%×[1−(final MTV−initial MTV of a treated group)/(final MTV−initial MTV of the control group)]) before any of the animals in the vehicle arm were euthanized due to a tumor size>1200 mm³. Statistical comparisons between the MTVs were conducted using one-way ANOVA followed by Tukey's multiple comparisons test comparing all groups. The P-values for each ADC compared to the vehicle control arm are reported. At the end of the study, efficacy was also determined in each treatment arm by counting the number of animals with a partial regression (PR) or a complete regression (CR) of the tumor. In a PR response, the tumor volume was 50% or less of its day 1 volume for 3 consecutive measurements during the course of study, and equal to or greater than 14 mm³ for 1 or more of these measurements. In a CR response, the tumor volume was less than 14 mm³ for 3 consecutive measurements. When an animal exhibited a CR response at the end of the study, it was classified as a tumor-free survivor (TFS) instead of a CR. Throughout the ADC studies no significant body weight changes due to ADC treatment were observed.

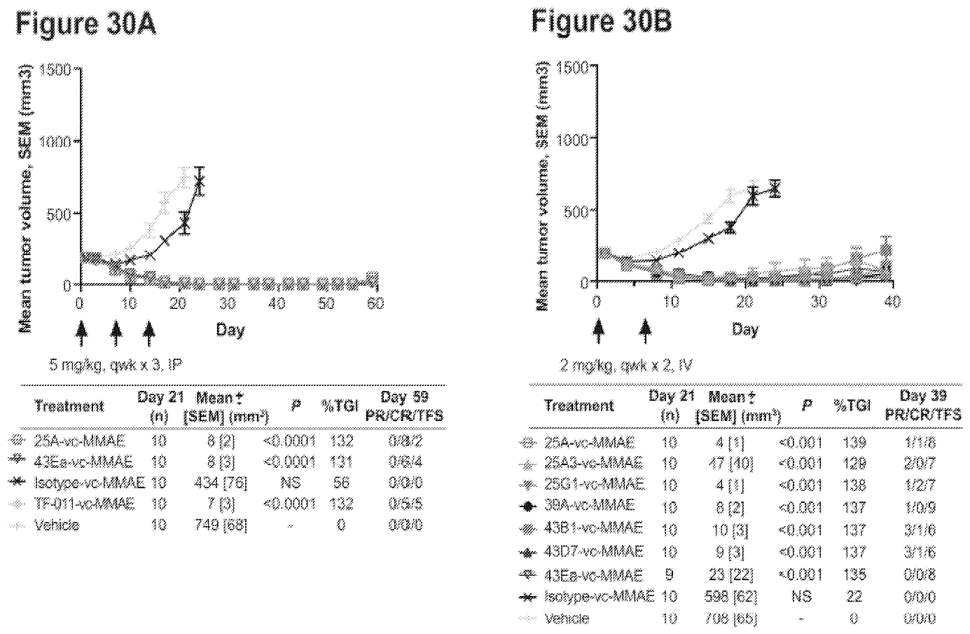

As shown in FIG. 30A, HPAF-II tumor-bearing mice were treated with 5 mg/kg of ADC on day 1, 8 and 15 after randomization. The effect of TF-011 ADC was compared with two representative clones from the two groups that did not impact coagulation (i.e. 25A and 43Ea). Twenty-one days after randomization the efficacy of the 25A, 43Ea and TF-011 ADCs was equivalent, with tumor growth inhibition ranging between 131 and 136%.

In the second HPAF-II study as shown in FIG. 30B, the highest affinity antibody that affected coagulation (i.e. 39A) and six antibodies with varying affinities from groups 25 and 43 (i.e. 25A, 25A3, 25G1, 43Ea, 43B1 and 43D7) were equally efficacious when dosed twice at 2 mg/kg. Tumor growth inhibition for the TF-specific ADCs ranged between 129 and 139% on day 21, and 6 to 9 out of 10 animals per treatment arm were classified as tumor-free survivors at the end of the study.

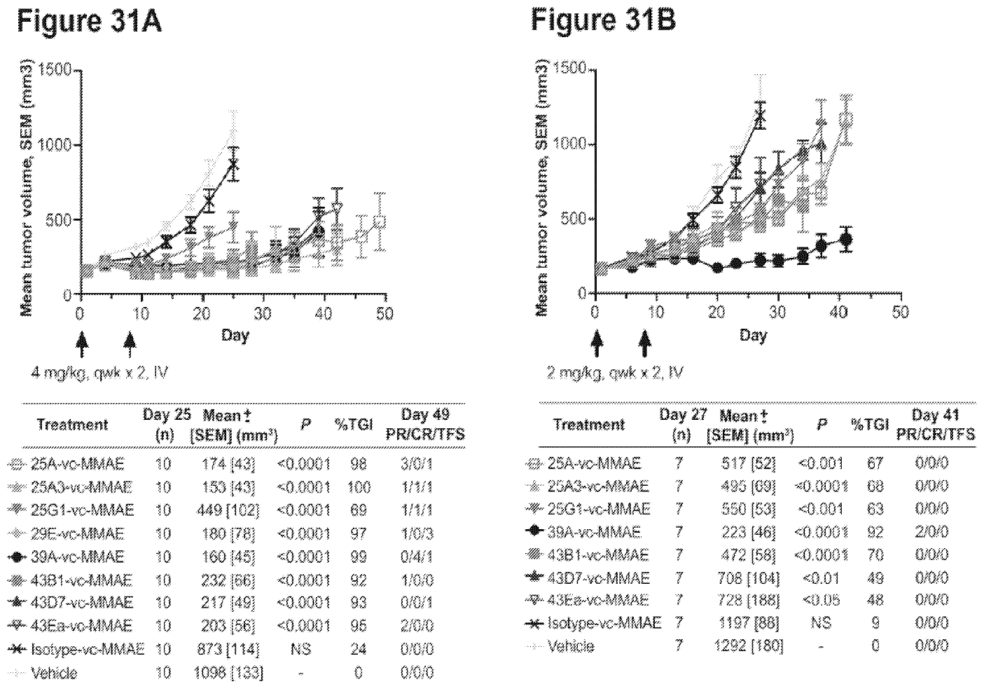

In the MDA-MB-231 xenograft model, the ADCs were administered on day 1 and 8 post-randomization at 4 or 2 mg/kg. As shown in FIG. 31A, all the TF-specific ADCs were active at 4 mg/kg, with tumor growth inhibition ranging between 69 and 100%, and a significant difference in mean tumor volume for each TF-specific ADC compared to the vehicle control arm. While a notable difference was observed in mean tumor volume between 25G1 and the other TF-specific ADCs, it was not statistically significant (P>0.05).

At 2 mg/kg of ADC as shown in FIG. 31B, all the TF-specific antibodies showed suboptimal activity with varying degrees of significance in mean tumor volume compared to the vehicle control arm. 25A3, 39A and 43B1 showed the greatest degree of significance in mean tumor volume compared to the vehicle control arm (P<1×10⁴). The difference in mean tumor volume between 39A and the other antibodies was only significant for the comparison between 39A and 43Ea (P<0.05).

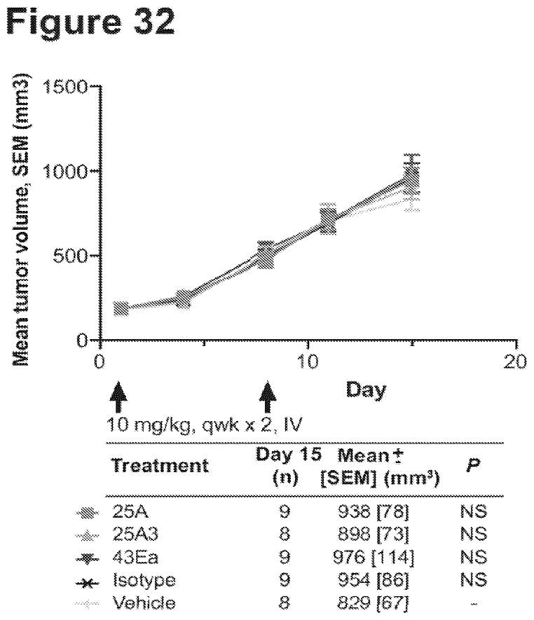

In contrast, the unconjugated antibodies of 25A, 25A3 and 43Ea lacked substantial anticancer activity when dosed twice at 10 mg/kg in the HPAF-II xenograft model (FIG. 32).

There results indicate that the TF-specific ADCs are efficacious in the HPAF-II and MDA-MB-231 xenograft model under various dosing regimens. The activity of the ADCs are caused by the toxin delivery of the anti-TF antibodies.

### Example 31: Studies in Patient-Derived Xenograft (PDX) Models

TF-positive PDX models were performed in athymic nude mice (Envigo, Indianapolis, IN) to evaluate the efficacy of the ADCs in vivo. The animals' care was in accordance with institutional guidelines. Study animals were implanted unilaterally on the left flank with tumor fragments.

For immunohistochemistry (IHC) analysis, tissues underwent pretreatment using Rip Tide (Mosaic Laboratories, Lake Forest, CA) for 40 min at 95-97° C. in a water bath, cooled for 10 min on the bench, rinsed 3 times with distilled water, and rinsed for 5 min with Splash-T Buffer (Mosaic Laboratories). Tissue sections were blocked in EnVision Peroxidase-Blocking Reagent (EnVision+Mouse HRP Detection Kit, Agilent, Carpinteria, CA) for 5 min, followed by 2 rinses in Splash-T Buffer for 5 min each. Next, the tissue sections were stained with an anti-TF antibody (mouse clone HTF-1) or a mouse negative control reagent for 30 min, followed by 2 rinses in Splash-T Buffer for 5 min each. The second staining step of the tissue sections was carried out for 30 min with EnVision+Mouse HRP (EnVision+Mouse HRP Detection Kit), followed by 2 rinses in Splash-T Buffer for 5 min each. To visualize the anti-TF staining, tissue sections were developed with DAB chromogen (EnVision+Mouse HRP Detection Kit) for 5 min, followed by 10 dips and a 5 min rinse in distilled water. Tissue sections were counterstained with Hematoxylin for 5 min followed by 3 rinses in distilled water.

Animals were randomized and treated as indicated in the figures. Animals were removed from study and euthanized once tumor size reached 1200 mm³ or skin ulceration was evident. In addition, the MTV curve for the treatment group in question was no longer shown once an animal was removed from study due to size. TGI and statistical analyses were conducted in the same manner as for the CDX studies. The CR and PR response definitions were as follows for the PDX studies: a PR responder had a MTV<30% of MTV at day 1 for 2 consecutive measurements; a CR responder had an undetectable MTV for 2 consecutive measurements. When an animal exhibited a CR response at the end of the study, it was classified as a TFS instead of a CR.

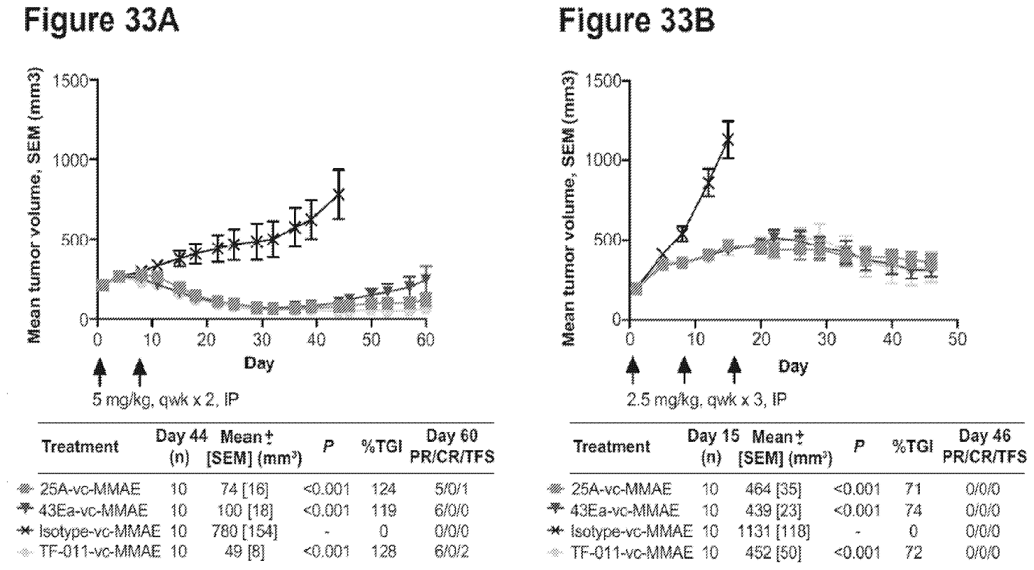

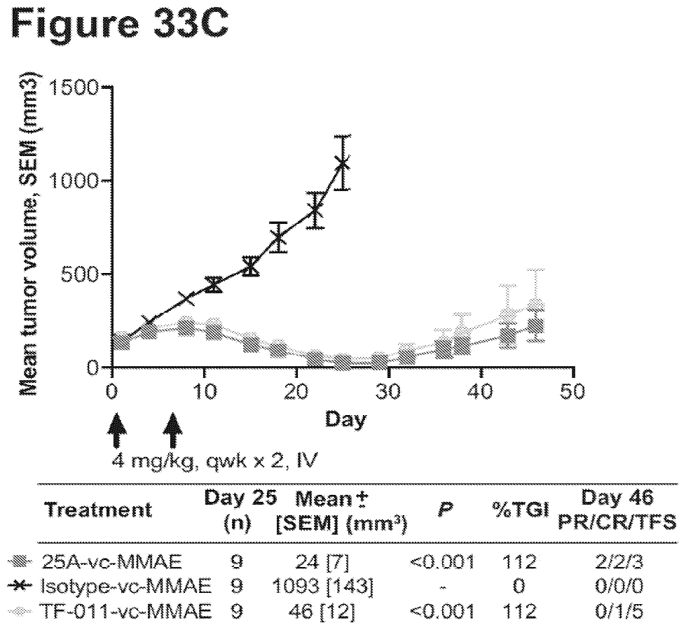

While the squamous cell carcinoma of the head and neck (SCCHN) and ovarian adenocarcinoma PDX had H-scores of 250 and 220, respectively, the gastric adenocarcinoma PDX had an H-score of 155 (data not shown). Upon randomization of tumor-bearing mice, treatment occurred on a weekly basis either twice or three times with the dose ranging between 2.5 and 5 mg/kg. As shown in FIGS. 33A, 33B, and 33C in all the PDX models a significant reduction in mean tumor volume was observed for each TF-specific ADC compared to the isotype control arm (P<1×10⁴), with no significant difference between the various TF-specific ADCs (P>0.05). In the head and neck and ovarian PDX model, the number of complete responders and tumor-free survivors did not exceed 2 out of 10 animals at the end of the study in any of the treatment groups (FIGS. 33A and 33B). However, in the gastric PDX the 25A treatment arm had 2 partial responders, 2 complete responders and 3 tumor-free survivors, and the TF-011 arm contained 1 complete responder and 5 tumor-free survivors at the end of the study (FIG. 33C).

These data indicate that the anti-TF ADCs from groups 25 and 43 (i.e. 25A and 43Ea) were equally efficacious as tisotumab vedotin (TF-011) ADC.

### Example 32: Efficacy Study in Swine CNV Model

An efficacy study in a swine choroidal neovascularization (CNV) model was performed to determine the effect 4 different anti-TF antibodies in reducing lesion size.

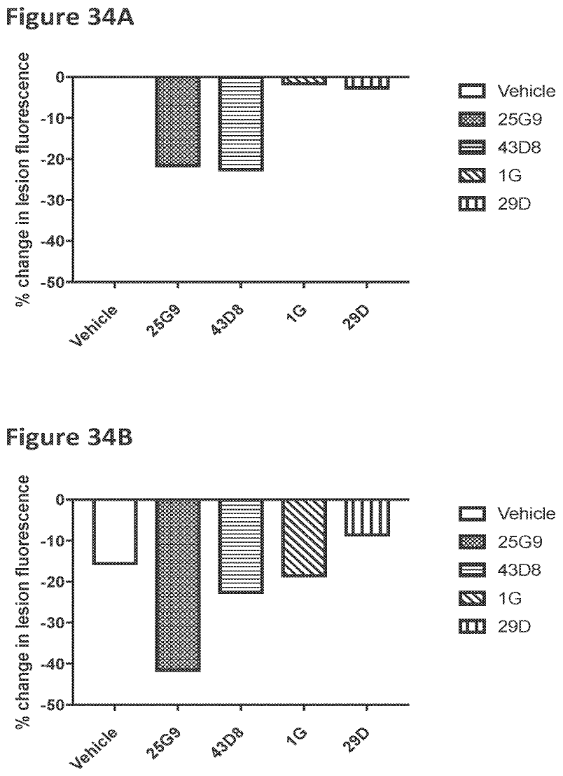

10-12 week old animals (Swine/Hampshire Cross) underwent bilateral laser using an 810 nm Diode laser delivered through an indirect ophthalmoscope to create approximately 6 single laser spots between retinal veins in each eye of each animal. For efficacy assessments, 2 mg of each anti-TF antibody, 25G9, 43D8, 1G, and 29D respectively, were administered intravitreally on day 7 post-laser treatment. A vehicle control group was also included in the study. Fluorescein Angiography (FA) to determine total lesion fluorescence was performed on day 7 (baseline), day 14 and day 28. FA was evaluated using a Corrected Total Lesion Fluorescence (CTLF) measurement for each individual lesion. The perimeter of the lesion was traced, and an integrated density value was obtained. CTLF was then calculated by subtracting the mean fluorescence background adjacent to the lesion from the integrated density measurement. Percent change in lesion size from day 7 to day 14 and from day 7 to day 28 are shown in FIG. 34A and FIG. 34B, respectively.

From day 7 to day 14, anti-hTF antibodies from groups 25 and 43, 25G9 and 43D8, reduced lesion size by greater than 20%. From day 7 to day 28, anti-hTF antibody 25G9 reduced lesion size by greater than 40%. Anti-hTF antibodies 1G and 29D did not reduce lesion size significantly as compared to the vehicle control group.

This data indicates that antibodies from groups 25 and 43, 25G9 and 43D8, were effective in reducing lesion size in a swine CNV model.

### Example 33: Efficacy of 25G9 in Swine CNV Model

An efficacy study in a swine choroidal neovascularization (CNV) model was performed to compare different doses of anti-TF antibody 25G9 for their ability to reduce lesion size.

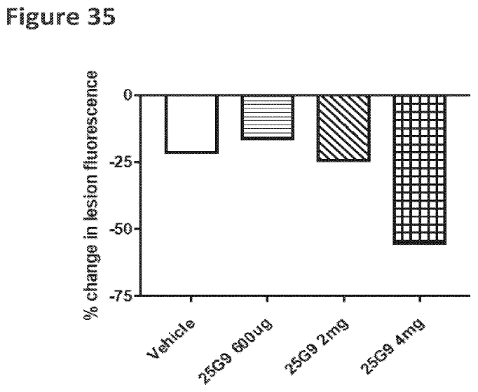

10-12 week old animals (Swine/Hampshire Cross) underwent bilateral laser using an 810 nm Diode laser delivered through an indirect ophthalmoscope to create approximately 6 single laser spots between retinal veins in each eye. For efficacy assessments 600 ug, 2 mg and 4 mg of anti-TF antibody 25G9 were administered intravitreally on Day 7 post-laser treatment. Fluorescein Angiography (FA) to determine total lesion fluorescence was performed on day 7 (baseline) and day 28. FA was evaluated using a Corrected Total Lesion Fluorescence (CTLF) measurement for each individual lesion. The perimeter of the lesion was traced, and an integrated density value was obtained. CTLF was then calculated by subtracting the mean fluorescence background adjacent to the lesion from the integrated density measurement. Percent changes in lesion size from day 7 to day 28 are shown in FIG. 35.

From day 7 to day 28, anti-hTF antibody 25G9 reduced lesion size in a dose-depenent matter. 25G9 reduced lesion size by greater than 50% at 4 mg. This data indicates that antibody 25G9 was effective in reducing lesion size in the swine CNV model in a dose-dependent matter.

### Example 34: Binding Affinity Assay for Pig TF and Rabbit TF

The ability of certain antibodies was tested for binding to pig TF. For pig TF Biacore-based measurements, a given anti-TF antibody was captured by an anti-human IgG antibody covalently coupled to a CM5 chip (GE Healthcare Bio-Sciences). Association between the anti-TF antibodies and a five-point three-fold titration of pig TF-His starting at 100 nM was measured for 180 to 240 sec. Subsequently, dissociation between the anti-TF antibody and TF-His was measured for 1800 sec. Kinetic data was analyzed and fitted globally using a 1:1 binding model. The K_{D} values of the indicated TF antibodies measured by the Biacore-based experiments are shown in Table 48.

The ability of certain antibodies was tested for binding to rabbit TF. For rabbit TF Biacore-based measurements, a given anti-TF antibody was captured by an anti-human IgG antibody covalently coupled to a CM5 chip (GE Healthcare Bio-Sciences). Association between the anti-TF antibodies and a five-point three-fold titration of rabbit TF-His starting at 100 nM was measured for 180 to 240 sec. Subsequently, dissociation between the anti-TF antibody and TF-His was measured for 1800 sec. Kinetic data was analyzed and fitted globally using a 1:1 binding model. The K_{D} values of the indicated TF antibodies measured by the Biacore-based experiments are shown in Table 48.

As shown in Table 48, anti-hTF antibodies from groups 25 and 43 exhibit binding activity and cross-reactivity to pig TF and rabbit TF. In contrast, antibodies from groups 1 and 29 show no binding activity to pig TF or rabbit TF.

TABLE 48
Antibody kinetics for pig and rabbit TF

| Antibody | Pig K_{D}, nM | Rabbit K_{D}, nM |
|---|---|---|
| 1G | no binding | no binding |
| 25A | 18.7 | 50.5 |
| 25A3 | 5.5 | 12.4 |
| 25A5 | 5.2 | 5.4 |
| 25A5-T | 4.5 | 5.4 |
| 25G | 26.0 | 75.5 |
| 25G1 | 2.6 | 3.6 |
| 25G9 | 3.3 | 4.2 |
| 29D | no binding | no binding |
| 43D7 | 8.8 | 6.8 |
| 43D8 | 19.2 | 7.7 |

no binding*: no binding to weak binding, with no reportable K_{D}

### Example 35: Immunohistochemistry (IHC) Assay

Tissues underwent pretreatment using Rip Tide (Mosaic Laboratories, Lake Forest, CA) for 40 min at 95-97° C. in a water bath, cooled for 10 min on the bench, rinsed 3 times with distilled water, and rinsed for 5 min with Splash-T Buffer (Mosaic Laboratories). Tissue sections were blocked in EnVision Peroxidase-Blocking Reagent (EnVision+Mouse HRP Detection Kit, Agilent, Carpinteria, CA) for 5 min, followed by 2 rinses in Splash-T Buffer for 5 min each. The tissue sections were then stained with an anti-TF antibody (mouse clone HTF-1) or a mouse negative control reagent for 30 min, followed by 2 rinses in Splash-T Buffer for 5 min each. The second staining step of the tissue sections was carried out for 30 min with EnVision+Mouse HRP (EnVision+Mouse HRP Detection Kit), followed by 2 rinses in Splash-T Buffer for 5 min each. To visualize the anti-TF staining, tissue sections were developed with DAB chromogen (EnVision+Mouse HRP Detection Kit) for 5 min, followed by 10 dips and a 5 min rinse in distilled water. Tissue sections were counterstained with Hematoxylin for 5 min followed by 3 rinses in distilled water.

Staining intensity was scored on a semi-quantitative integer scale from 0 (negative) to 3 (or “3+”) by a certified anatomic pathologist. The percentage of cells staining positively at each intensity level was recorded. Scoring was based on localization of TF to the cell membrane. The H score combines components of staining intensity with the percentage of positive cells. It has a value between 0 and 300 and is defined as: 1×(percentage of cells staining at 1+intensity)+2×(percentage of cells staining at 2+intensity)+3×(percentage of cells staining at 3+intensity)=H score.

Tissue sections from patients with kidney cancer, head & neck cancer, ovarian cancer, gastric cancer, prostate cancer, gastroesophageal junction cancer, cervical cancer, and glioblastoma were stained. The number of patients with scores within each H score range and the total number of patient for each cancer are shown in Table 50. These results indicate that TF is expressed in kidney cancer, head & neck cancer, ovarian cancer, gastric cancer, prostate cancer, gastroesophageal junction cancer, cervical cancer, and glioblastoma.

TABLE 50
Results of IHC assay

| | H score | H score | H score | H score |
|---|---|---|---|---|
| Indication | 0 | 1-100 | 101-200 | 201-300 |
| Kidney | 19/28 | 6/28 | 1/28 | 2/28 |
| Head & Neck | 4/74 | 31/74 | 19/74 | 20/74 |
| Ovarian | 13/26 | 11/26 | 0/26 | 2/26 |
| Gastric | 1/20 | 9/20 | 4/20 | 6/20 |
| Prostrate | 1/24 | 8/24 | 7/24 | 8/24 |
| Pancreatic | 14/37 | 18/37 | 5/37 | 0/37 |
| Gastroesophageal | 28/59 | 23/59 | 6/59 | 2/59 |
| junction | | | | |
| Cervical | 31/60 | 21/60 | 7/60 | 1/60 |
| Glioblastoma | 2/41 | 7/41 | 23/41 | 9/41 |

### Example 36: Epitope Binning of Anti-TF Antibodies

To establish epitope binding differences between the anti-human TF antibodies, chimeric TF construct mapping experiments were conducted. This mapping technique enables discrimination of antibody epitopes.

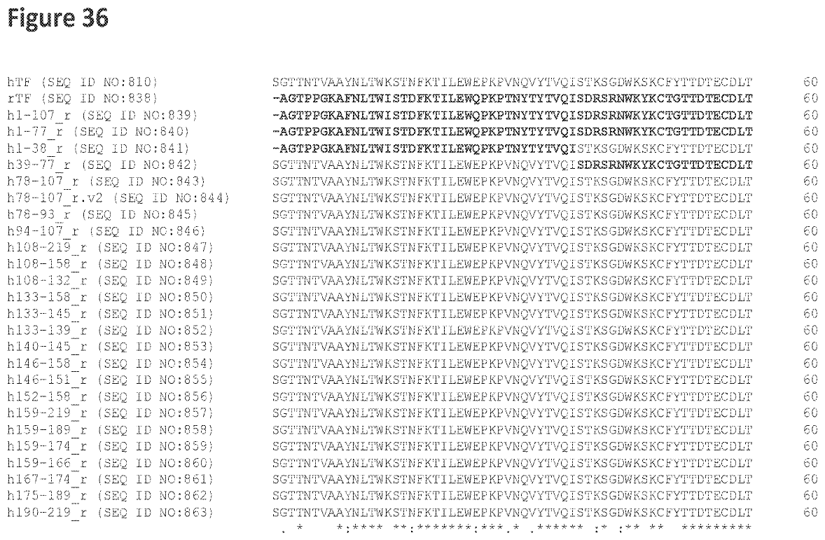

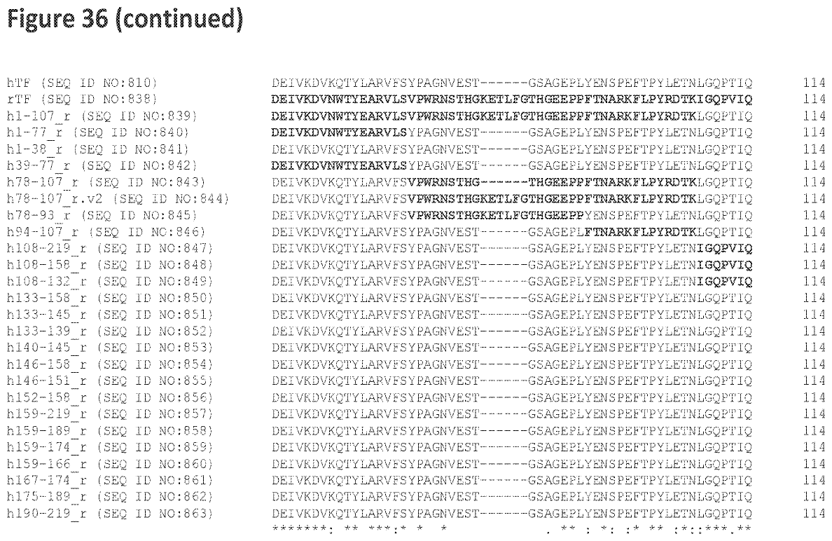

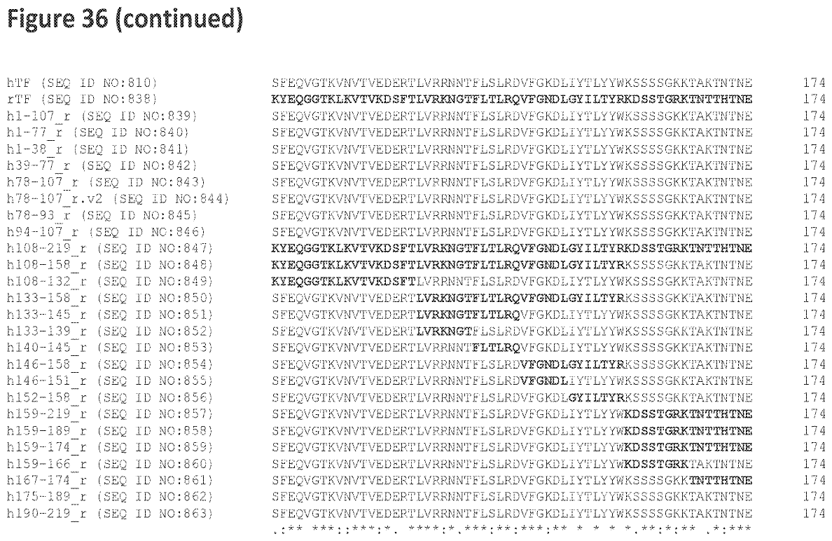

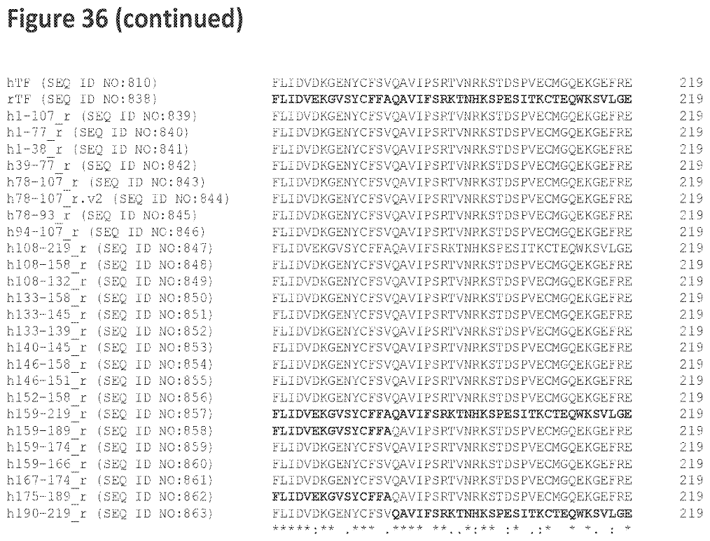

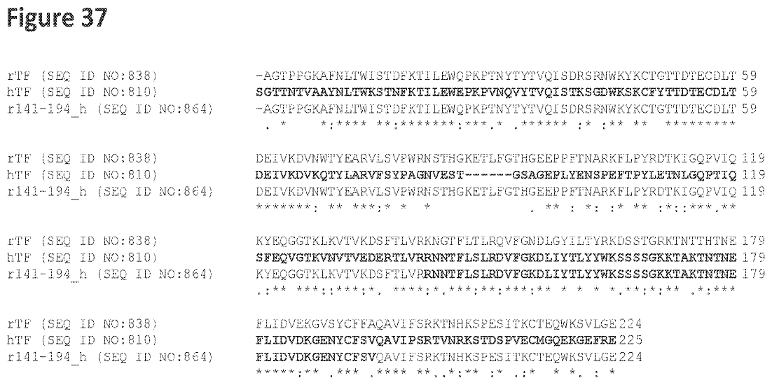

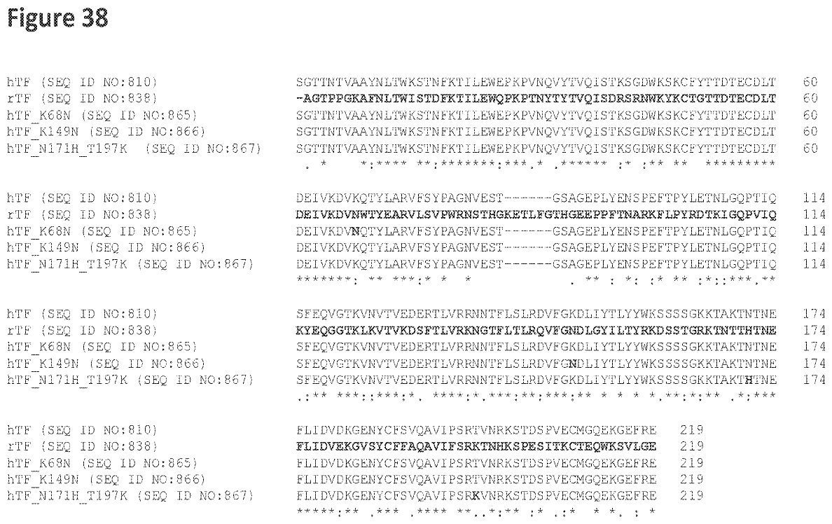

Because all the anti-human TF antibodies evaluated do not bind rat TF, the rat TF sequence was used for the construction of chimeric human-rat TF constructs. Chimeric human-rat construct design was guided by the N- and C-terminal domain of TF extracellular domain (amino acids 1-107 and 108-219 of the extracellular domain, respectively), with an alignment shown in FIG. 36. Based on the chimera mapping results using the constructs from FIG. 36, rat amino acid segment 141-194 was replaced by the human sequence (amino acid 136-189 of hTF extracelluar domain), with an alignment shown in FIG. 37. Design of three human TF constructs with either 1 or 2 human-rat substitutions (hTF_K68N, hTF_K149N and hTF_N171H_T197K) was based on reported contact residues K68, K149 and N171 and T197 for the 10H10 antibody (Teplyakov et al., Cell Signal., 2017, 36:139-144), with an alignment shown in FIG. 38.

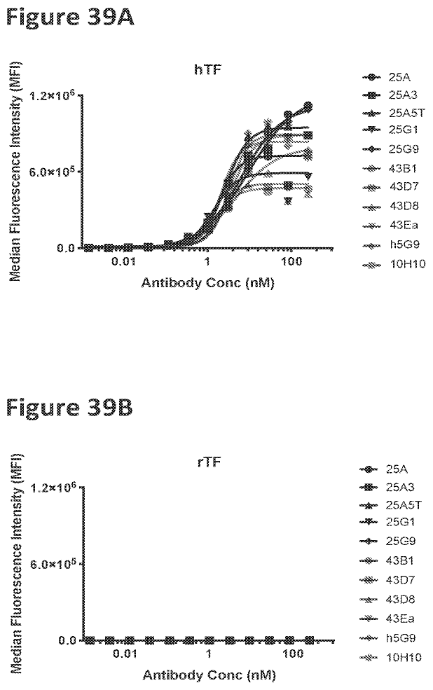

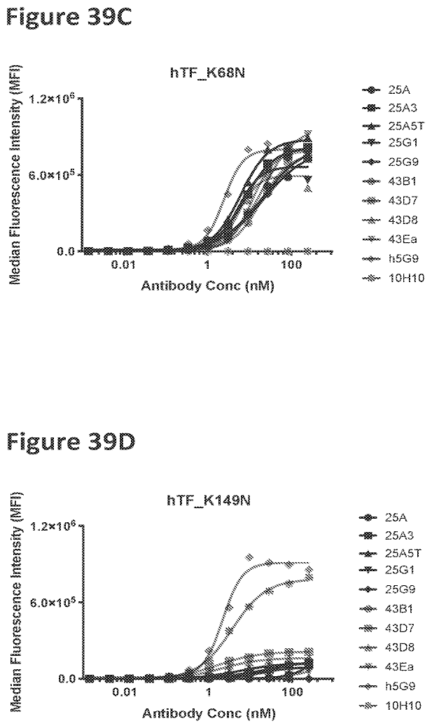

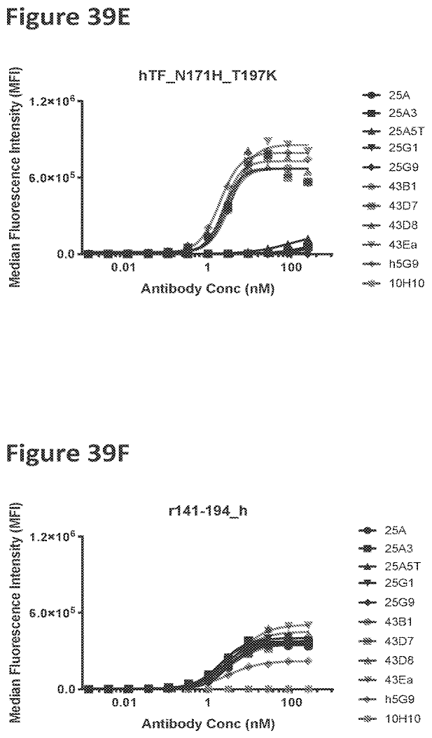

To establish binding of the anti-human TF antibodies to the various TF constructs, HEK293 cells were transfected with a DNA plasmid that co-expresses the TF construct and a green fluorescent protein marker. For a subset of the antibodies, an antibody titration (a 12-point 1:3 dilution series starting at 250 nM) was evaluated on select TF constructs (FIGS. 39A-F). These antibody titrations demonstrated that the antibody concentration of 15 μg/ml (100 nM) used in Tables 51 and 52 was appropriate to establish “Percentage antibody binding to TF construct relative to hTF”. Two days after transfection, cells were collected from the tissue culture plate, stained with 15 μg/ml of the indicated anti-TF antibody, washed, stained with anti-human IgG-Fc Alexa Fluor 647 polyclonal antibody, washed, and stained with the viability dye 4′,6-Diamidino-2-Phenylindole, Dihydrochloride. Upon acquisition of 80,000 live events on a flow cytometer, live cells marked with the fluorescent marker were analyzed for the degree of staining by the anti-TF antibody. The median fluorescence intensity values relative to an isotype control for each TF expression construct were divided by the median fluorescence intensity value relative to an isotype control for the hTF expression construct, and the resulting percentage listed as “Percentage antibody binding to TF construct relative to hTF” in Tables 51 and 52. As used herein, the term “live cell staining assay” refers to the antibody binding assay used in this example.

The assumption that all chimeric TF constructs were expressed on the cell surface at levels between 50% and 150% of the hTF control construct was met for all TF constructs for at least one anti-human TF antibody in the antibody collection, with the exception of the h1-107_r construct (human amino acid segment 1-107 replaced by rat sequence). Lack of binding of the anti-human TF antibodies to cell surface-expressed rat TF was expected. When “Percentage antibody binding to TF construct relative to hTF” in Tables 51 and 52 was less than 50%, the antibody was considered a non-binder (0) in Tables 53 and 54. When “Percentage antibody binding to TF construct relative to hTF” in Tables 51 and 52 was between 50% and 150%, the antibody was considered a binder (1) in Tables 53 and 54.

Each antibody was assigned to an epitope bin in Table 55 based on the combination of unbound constructs from Table 53. The antibodies from Lineage 25 (25A, 25A3, 25A5-T, 25G1 and 25G9) bind a unique epitope, referred to as Epitope Bin 6 in Table 55. The antibodies from Lineage 43 (43B1, 43D7, 43D8 and 43Ea) also bind a unique epitope, referred to as Epitope Bin 7 in Table 55. The antibody from Lineage 29 (29E) binds a unique epitope, referred to as Epitope Bin 2 in Table 55. The antibodies from Lineage 39 and 54 (39A and 54E) bind a unique epitope, referred to as Epitope Bin 3 in Table 55.

Lineage 25 and 43 antibodies are the only antibodies in the antibody panel that bind r141-194_h, the chimeric construct in which rat amino acids 141-194 were replaced by human sequence (FIG. 39F; Table 54). Furthermore, while M1593 cannot bind hTF_K68N, all the other antibodies in the antibody panel bind hTF_K68N (FIG. 39C; Table 54). Only Lineage 25 and 43 antibodies cannot bind hTF_K149N (FIG. 39D; Table 54). Only Lineage 25 antibodies cannot bind hTF_N171H_T197K (FIG. 39E; Table 54).

In summary, these results indicate that lineage 25 antibodies bind a unique epitope on human TF compared to all other antibodies tested. Lineage 43 antibodies bind a unique epitope on human TF compared to all other antibodies tested. Lineage 25 and lineage 43 antibodies bind a different epitope on human TF from M1593.

TABLE 51
Percent antibody binding to TF construct relative to hTF

| | | Antibody | | | | | | | | | | | | | | | |
|---|---|---|---|---|---|---|---|---|---|---|---|---|---|---|---|---|---|
| | Construct | 1F | 29E | 39A | 54E | TF-011 | 5G9 | M1593 | 25A | 25A3 | 25A5-T | 25G1 | 25G9 | 43B1 | 43D7 | 43D8 | 43Ea |
| | hTF | 100 | 100 | 100 | 100 | 100 | 100 | 100 | 100 | 100 | 100 | 100 | 100 | 100 | 100 | 100 | 100 |
| | rTF | 0 | 0 | 0 | 0 | 0 | 0 | 0 | 0 | 0 | 0 | 0 | 0 | 0 | 0 | 0 | 0 |
| Human | h1-107_r (52) | 0 | 0 | 0 | 0 | 0 | 41 | 0 | 32 | 36 | 36 | 37 | 28 | 33 | 35 | 31 | 37 |
| amino | h1-77_r (25) | 0 | 0 | 0 | 0 | 0 | 94 | 0 | 86 | 95 | 84 | 88 | 64 | 64 | 75 | 69 | 69 |
| acid | h1-38_r (14) | 91 | 87 | 100 | 102 | 104 | 100 | 104 | 101 | 104 | 93 | 101 | 88 | 97 | 106 | 104 | 103 |
| segment | h39-77_r (11) | 0 | 0 | 0 | 0 | 0 | 88 | 2 | 82 | 88 | 80 | 87 | 71 | 59 | 75 | 71 | 69 |
| replaced | h78-107_r (21) | 0 | 8 | 81 | 68 | 32 | 114 | 74 | 108 | 116 | 103 | 113 | 108 | 113 | 114 | 117 | 114 |
| by rat | h78-107_ | 0 | 0 | 76 | 62 | 23 | 101 | 59 | 95 | 96 | 91 | 94 | 93 | 97 | 100 | 101 | 101 |
| segment (in | r.v2 (27) | | | | | | | | | | | | | | | | |
| parentheses: | h78-93_r (18) | 102 | 0 | 77 | 91 | 110 | 102 | 104 | 106 | 105 | 92 | 101 | 98 | 101 | 104 | 102 | 103 |
| number of | h94-107_r (9) | 1 | 82 | 85 | 89 | 27 | 91 | 46 | 82 | 86 | 78 | 83 | 77 | 84 | 92 | 89 | 91 |
| amino acid | h108-219_r (46) | 119 | 118 | 118 | 122 | 128 | 0 | 0 | 0 | 0 | 0 | 0 | 0 | 0 | 0 | 0 | 0 |
| changes | h108-158_r (19) | 98 | 101 | 107 | 108 | 108 | 63 | 4 | 1 | 0 | 0 | 11 | 22 | 0 | 1 | 0 | 0 |
| relative to | h108-132_r (10) | 105 | 108 | 109 | 107 | 124 | 125 | 124 | 112 | 112 | 106 | 111 | 118 | 122 | 126 | 122 | 124 |
| human TF) | h133-158_r (9) | 113 | 122 | 119 | 130 | 134 | 91 | 0 | 0 | 0 | 0 | 2 | 3 | 0 | 4 | 1 | 0 |
| | h133-145_r (4) | 84 | 95 | 96 | 104 | 104 | 108 | 100 | 77 | 80 | 80 | 87 | 100 | 99 | 104 | 103 | 106 |
| | h133-139_r (2) | 82 | 90 | 95 | 103 | 102 | 104 | 103 | 88 | 89 | 88 | 91 | 86 | 94 | 101 | 97 | 101 |
| | h140-145_r (2) | 89 | 100 | 101 | 110 | 109 | 113 | 97 | 80 | 87 | 86 | 89 | 105 | 101 | 104 | 104 | 109 |
| | h146-158_r (5) | 115 | 122 | 125 | 134 | 134 | 91 | 133 | 2 | 17 | 18 | 17 | 0 | 3 | 20 | 10 | 0 |
| | h146-151_r (1) | 122 | 133 | 139 | 142 | 143 | 141 | 118 | 3 | 14 | 17 | 7 | 0 | 11 | 39 | 23 | 2 |
| | h152-158_r (4) | 110 | 121 | 128 | 127 | 136 | 82 | 132 | 110 | 116 | 112 | 116 | 111 | 119 | 134 | 129 | 134 |
| | h159-219_r (27) | 132 | 134 | 141 | 142 | 155 | 0 | 137 | 0 | 0 | 0 | 0 | 0 | 132 | 130 | 130 | 76 |
| | h159-189_r (11) | 94 | 101 | 104 | 110 | 112 | 0 | 105 | 0 | 0 | 0 | 0 | 0 | 100 | 106 | 104 | 94 |
| | h159-174_r (6) | 96 | 98 | 101 | 118 | 120 | 0 | 98 | 0 | 0 | 0 | 0 | 0 | 103 | 115 | 112 | 101 |
| | h159-166_r (3) | 89 | 93 | 96 | 100 | 98 | 104 | 100 | 93 | 95 | 87 | 91 | 88 | 99 | 106 | 105 | 110 |
| | h167-174_r (3) | 96 | 112 | 96 | 122 | 128 | 0 | 118 | 0 | 0 | 0 | 0 | 0 | 109 | 121 | 112 | 104 |
| | h175-189_r (5) | 97 | 113 | 112 | 118 | 123 | 119 | 114 | 86 | 95 | 99 | 100 | 86 | 109 | 118 | 114 | 122 |
| | h190-219_r (16) | 111 | 138 | 149 | 141 | 145 | 12 | 143 | 125 | 124 | 119 | 127 | 144 | 133 | 140 | 136 | 147 |

TABLE 52
Percent antibody binding to TF construct relative to hTF

| | | Antibody | | | | | | | | | | | | | | |
|---|---|---|---|---|---|---|---|---|---|---|---|---|---|---|---|---|
| Construct | 1F | 29E | 39A | 54E | TF-011 | 5G9 | M1593 | 25A | 25A3 | 25A5-T | 25G1 | 25G9 | 43B1 | 43D7 | 43D8 | 43Ea |
| hTF | 100 | 100 | 100 | 100 | 100 | 100 | 100 | 100 | 100 | 100 | 100 | 100 | 100 | 100 | 100 | 100 |
| rTF | 0 | 0 | 0 | 0 | 0 | 0 | 0 | 0 | 0 | 0 | 0 | 0 | 0 | 0 | 0 | 0 |
| r141-194_h* | 0 | 0 | 0 | 0 | 0 | 32 | 0 | 65 | 89 | 88 | 83 | 108 | 90 | 102 | 95 | 81 |
| hTF_K68N | 105 | 115 | 119 | 118 | 111 | 132 | 0 | 93 | 124 | 126 | 115 | 103 | 107 | 116 | 119 | 118 |
| hTF_K149N | 115 | 117 | 131 | 127 | 132 | 145 | 111 | 2 | 12 | 13 | 7 | 0 | 10 | 29 | 20 | 1 |
| hTF_N171H_T197K | 83 | 98 | 94 | 89 | 109 | 102 | 113 | 1 | 4 | 7 | 1 | 0 | 98 | 101 | 103 | 118 |

*rat amino acid segment replaced by human segment, resulting in 20 amino acid changes

TABLE 53
Antibody binding to TF construct

| | | Antibody | | | | | | | | | | | | | | | |
|---|---|---|---|---|---|---|---|---|---|---|---|---|---|---|---|---|---|
| | | | | | | TF- | | | | | | | | | | | |
| | Construct | 1F | 29E | 39A | 54E | 011 | 5G9 | M1593 | 25A | 25A3 | 25A5-T | 25G1 | 25G9 | 43B1 | 43D7 | 43D8 | 43Ea |
| | hTF | 1 | 1 | 1 | 1 | 1 | 1 | 1 | 1 | 1 | 1 | 1 | 1 | 1 | 1 | 1 | 1 |
| | rTF | 0 | 0 | 0 | 0 | 0 | 0 | 0 | 0 | 0 | 0 | 0 | 0 | 0 | 0 | 0 | 0 |
| Human amino | h1-107_r (52) | 0 | 0 | 0 | 0 | 0 | 0 | 0 | 0 | 0 | 0 | 0 | 0 | 0 | 0 | 0 | 0 |
| acid segment | h1-77_r (25) | 0 | 0 | 0 | 0 | 0 | 1 | 0 | 1 | 1 | 1 | 1 | 1 | 1 | 1 | 1 | 1 |
| replaced by | h1-38_r (14) | 1 | 1 | 1 | 1 | 1 | 1 | 1 | 1 | 1 | 1 | 1 | 1 | 1 | 1 | 1 | 1 |
| rat segment | h39-77_r (11) | 0 | 0 | 0 | 0 | 0 | 1 | 0 | 1 | 1 | 1 | 1 | 1 | 1 | 1 | 1 | 1 |
| (in parentheses: | h78-107_r (21) | 0 | 0 | 1 | 1 | 0 | 1 | 1 | 1 | 1 | 1 | 1 | 1 | 1 | 1 | 1 | 1 |
| number of | h78-107_r.v2 (27) | 0 | 0 | 1 | 1 | 0 | 1 | 1 | 1 | 1 | 1 | 1 | 1 | 1 | 1 | 1 | 1 |
| amino acid | h78-93_r (18) | 1 | 0 | 1 | 1 | 1 | 1 | 1 | 1 | 1 | 1 | 1 | 1 | 1 | 1 | 1 | 1 |
| changes relative | h94-107_r (9) | 0 | 1 | 1 | 1 | 0 | 1 | 0 | 1 | 1 | 1 | 1 | 1 | 1 | 1 | 1 | 1 |
| to human TF) | h108-219_r (46) | 1 | 1 | 1 | 1 | 1 | 0 | 0 | 0 | 0 | 0 | 0 | 0 | 0 | 0 | 0 | 0 |
| | h108-158_r (19) | 1 | 1 | 1 | 1 | 1 | 1 | 0 | 0 | 0 | 0 | 0 | 0 | 0 | 0 | 0 | 0 |
| | h108-132_r (10) | 1 | 1 | 1 | 1 | 1 | 1 | 1 | 1 | 1 | 1 | 1 | 1 | 1 | 1 | 1 | 1 |
| | h133-158_r (9) | 1 | 1 | 1 | 1 | 1 | 1 | 0 | 0 | 0 | 0 | 0 | 0 | 0 | 0 | 0 | 0 |
| | h133-145_r (4) | 1 | 1 | 1 | 1 | 1 | 1 | 1 | 1 | 1 | 1 | 1 | 1 | 1 | 1 | 1 | 1 |
| | h133-139_r (2) | 1 | 1 | 1 | 1 | 1 | 1 | 1 | 1 | 1 | 1 | 1 | 1 | 1 | 1 | 1 | 1 |
| | h140-145_r (2) | 1 | 1 | 1 | 1 | 1 | 1 | 1 | 1 | 1 | 1 | 1 | 1 | 1 | 1 | 1 | 1 |
| | h146-158_r (5) | 1 | 1 | 1 | 1 | 1 | 1 | 1 | 0 | 0 | 0 | 0 | 0 | 0 | 0 | 0 | 0 |
| | h146-151_r (1) | 1 | 1 | 1 | 1 | 1 | 1 | 1 | 0 | 0 | 0 | 0 | 0 | 0 | 0 | 0 | 0 |
| | h152-158_r (4) | 1 | 1 | 1 | 1 | 1 | 1 | 1 | 1 | 1 | 1 | 1 | 1 | 1 | 1 | 1 | 1 |
| | h159-219_r (27) | 1 | 1 | 1 | 1 | 1 | 0 | 1 | 0 | 0 | 0 | 0 | 0 | 1 | 1 | 1 | 1 |
| | h159-189_r (11) | 1 | 1 | 1 | 1 | 1 | 0 | 1 | 0 | 0 | 0 | 0 | 0 | 1 | 1 | 1 | 1 |
| | h159-174_r (6) | 1 | 1 | 1 | 1 | 1 | 0 | 1 | 0 | 0 | 0 | 0 | 0 | 1 | 1 | 1 | 1 |
| | h159-166_r (3) | 1 | 1 | 1 | 1 | 1 | 1 | 1 | 1 | 1 | 1 | 1 | 1 | 1 | 1 | 1 | 1 |
| | h167-174_r (3) | 1 | 1 | 1 | 1 | 1 | 0 | 1 | 0 | 0 | 0 | 0 | 0 | 1 | 1 | 1 | 1 |
| | h175-189_r (5) | 1 | 1 | 1 | 1 | 1 | 1 | 1 | 1 | 1 | 1 | 1 | 1 | 1 | 1 | 1 | 1 |
| | h190-219_r (16) | 1 | 1 | 1 | 1 | 1 | 0 | 1 | 1 | 1 | 1 | 1 | 1 | 1 | 1 | 1 | 1 |

TABLE 54
Antibody binding to TF construct

| | Antibody | | | | | | | | | | | | | | | |
|---|---|---|---|---|---|---|---|---|---|---|---|---|---|---|---|---|
| Construct | 1F | 29E | 39A | 54E | TF-011 | 5G9 | M1593 | 25A | 25A3 | 25A5-T | 25G1 | 25G9 | 43B1 | 43D7 | 43D8 | 43Ea |
| hTF | 1 | 1 | 1 | 1 | 1 | 1 | 1 | 1 | 1 | 1 | 1 | 1 | 1 | 1 | 1 | 1 |
| rTF | 0 | 0 | 0 | 0 | 0 | 0 | 0 | 0 | 0 | 0 | 0 | 0 | 0 | 0 | 0 | 0 |
| r141-194_h* | 0 | 0 | 0 | 0 | 0 | 0 | 0 | 1 | 1 | 1 | 1 | 1 | 1 | 1 | 1 | 1 |
| hTF_K68N | 1 | 1 | 1 | 1 | 1 | 1 | 0 | 1 | 1 | 1 | 1 | 1 | 1 | 1 | 1 | 1 |
| hTF_K149N | 1 | 1 | 1 | 1 | 1 | 1 | 1 | 0 | 0 | 0 | 0 | 0 | 0 | 0 | 0 | 0 |
| hTF_N171H_T197K | 1 | 1 | 1 | 1 | 1 | 1 | 1 | 0 | 0 | 0 | 0 | 0 | 1 | 1 | 1 | 1 |

*rat amino acid segment replaced by human segment, resulting in 20 amino acid changes

TABLE 55
Epitope Bin assignment based on unbound chimeric constructs

| Antibody | Constructs not bound by antibody | Epitope Bin |
|---|---|---|
| 1F | rTF, h1-107_r, h1-77_r, h39-77_r, h78-107_r, h78-107_r.v2. h94-107_r | 1 |
| 29E | rTF, h1-107_r, h1-77_r, h39-77_r, h78-107_r, H78-107_r.v2, h78-93_r | 2 |
| 39A | rTF, h1-107_r, h1-77_r, h39-77_r | 3 |
| 54E | rTF, h1-107_r, h1-77_r, h39-77_r | 3 |
| TF-011 | rTF, h1-107_r, h1-77_r, h39-77_r, h78-107_r, h78-107_r.v2, h94-107_r | 1 |
| 5G9 | rTF, h1-107_r, h108-219_r, h159-219_r, h159-189_r, h159-174_r, h 167-174_r, | 4 |
| | h190-219_r | |
| M1593 | rTF, h1-107_r, h1-77_r, h39-77_r, h94-107_r, h108-219_r, h 108-158_r, | 5 |
| | h133-158_r | |
| 25A | rTF, h1-107_r, h108-219_r, h108-158_r, h133-158_r, h146-158_r, h146-151_r, | 6 |
| | h159-219_r, h159-189_r, h159-174_r, h167-174_r | |
| 25A3 | rTF, h1-107_r, h108-219_r, h108-158_r, h133-158_r, h146-158_r, h146-151_r, | 6 |
| | h159-219 r, h159-189_r, h159-174_r, h167-174_r | |
| 25A5-T | rTF, h1-107_r, h108-219_r, h108-158_r, h133-158_r, h146-158_r, h146-151_r, | 6 |
| | h159-219_r, h 159-189_r, h159-174_r, h167-174_r | |
| 25G1 | rTF, h1-107_r, h108-219_r, h108-158_r, h133-158_r, h146-158_r, h146-151_r, | 6 |
| | h159-219_r, h159-189_r, h159-174_r, h167-174_r | |
| 25G9 | rTF, h1-107_r, h108-219_r, h108-158_r, h133-158_r, h146-158_r, h146-151_r, | 6 |
| | h159-219_r, h159-189_r, h159-174 r, h167-174_r | |
| 43B1 | rTF, h1-107_r, h108-219_r, h108-158_r, h133-158_r, h146-158_r, h146-151_r | 7 |
| 43D7 | rTF, h1-107_r, h108-219_r, h108-158_r, h133-158_r, h146-158_r, h146-151_r | 7 |
| 43D8 | rTF, h1-107_r, h108-219_r, h108-158_r, h133-158_r, h146-158_r, h146-151_r | 7 |
| 43Ea | rTF, h1-107_r, h108-219_r, h108-158_r, h133-158_r, h146-158_r, h146-151_r | 7 |

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### Sequences

TABLE 13
Variable region sequences

| Clone | VH Domains (SEQ ID NO) | VL Domains (SEQ ID NO) |
|---|---|---|
| 1F | EVQLLESGGGLVQPGGSLRLSCAASG | DIQMTQSPSTLSASVGDRVTITCRASQ |
| | FTFSDYAMGWVRQAPGKGLEWVSTIS | SISSWLAWYQQKPGKAPKLLIYKASSL |
| | GSGGLTYYADSVKGRFTISRDNSKNT | ESGVPSRFSGSGSGTEFTLTISSLQPD |
| | LYLQMNSLRAEDTAVYYCAKAPYGYY | DFATYYCQQYKSYITFGGGTKVEIK |
| | MDVWGKGTTVTVSS(SEQ ID NO: | (SEQ ID NO: 38) |
| | 37) | |
| 1G | EVQLLESGGGLVQPGGSLRLSCAASG | DIQMTQSPSTLSASVGDRVTITCRASQ |
| | FTFSSYAMAWVRQAPGKGLEWVSAIS | SISSWLAWYQQKPGKAPKLLIYKASSL |
| | GSGGLTYYADSVKGRFTISRDNSKNT | ESGVPSRFSGSGSGTEFTLTISSLQPD |
| | LYLQMNSLRAEDTAVYYCAKAPYGYY | DFATYYCQQYKSYITFGGGTKVEIK |
| | MDVWGKGTTVTVSS(SEQ ID NO: | (SEQ ID NO: 76) |
| | 75) | |
| 25A | QVQLVQSGAEVKKPGASVKVSCKASG | DIQMTQSPSTLSASVGDRVTITCRASQ |
| | YTFDVYGISWVRQAPGQGLEWMGWIA | SISSWLAWYQQKPGKAPKLLIYKASSL |
| | PYNGNTNYAQKLQGRVTMTTDTSTST | ESGVPSRFSGSGSGTEFTLTISSLQPD |
| | AYMELRSLRSDDTAVYYCARDAGTYS | DFATYYCQQFQSLPPFTFGGGTKVEIK |
| | PFGYGMDVWGQGTTVTVSS(SEQ ID | (SEQ ID NO: 114) |
| | NO: 113) | |
| 25A3 | QVQLVQSGAEVKKPGASVKVSCKASG | DIQMTQSPSTLSASVGDRVTITCQASQ |
| | YTFDVYGISWVRQAPGQGLEWMGWIA | SINNWLAWYQQKPGKAPKLLIYKAYNL |
| | PYSGNTNYAQKLQGRVTMTTDTSTST | ESGVPSRFSGSGSGTEFTLTISSLQPD |
| | AYMELRSLRSDDTAVYYCARDAGTYS | DFATYYCQLFQSLPPFTFGGGTKVEIK |
| | PFGYGMDVWGQGTTVTVSS(SEQ ID | (SEQ ID NO: 152) |
| | NO: 151) | |
| 25A5 | QVQLVQSGAEVKKPGASVKVSCKASG | DIQMTQSPSTLSASVGDRVTITCRASE |
| | YTFDVYGISWVRQAPGQGLEWMGWIA | SISNWLAWYQQKPGKAPKLLIYKAYSL |
| | PYSGNTNYAQKLQGRVTMTTDTSTST | EYGVPSRFSGSGSGTEFTLTISSLQPD |
| | AYMELRSLRSDDTAVYYCARDAGTYS | DFATYYCQQFQKLPPFTFGGGTKVEIK |
| | PFGYGMDVWGQGTTVTVSS(SEQ ID | (SEQ ID NO: 190) |
| | NO: 189) | |
| 25A5-T | QVQLVQSGAEVKKPGASVKVSCKASG | DIQMTQSPSTLSASVGDRVTITCRASE |
| | YTFDAYGISWVRQAPGQGLEWMGWIA | SISNWLAWYQQKPGKAPKLLIYKAYSL |
| | PYSGNTNYAQKLQGRVTMTTDTSTST | EYGVPSRFSGSGSGTEFTLTISSLQPD |
| | AYMELRSLRSDDTAVYYCARDAGTYS | DFATYYCQQFQKLPPFTFGGGTKVEIK |
| | PFGYGMDVWGQGTTVTVSS(SEQ ID | (SEQ ID NO: 837) |
| | NO: 836) | |
| 25G | QVQLVQSGAEVKKPGASVKVSCKASG | DIQMTQSPSTLSASVGDRVTITCRASQ |
| | YTFRSYGISWVRQAPGQGLEWMGWVA | SISSWLAWYQQKPGKAPKLLIYKASSL |
| | PYNGNTNYAQKLQGRVTMTTDTSTST | ESGVPSRFSGSGSGTEFTLTISSLQPD |
| | AYMELRSLRSDDTAVYYCARDAGTYS | DFATYYCQQFQSLPPFTFGGGTKVEIK |
| | PYGYGMDVWGQGTTVTVSS(SEQ ID | (SEQ ID NO: 228) |
| | NO: 227) | |
| 25G1 | QVQLVQSGAEVKKPGASVKVSCKASG | DIQMTQSPSTLSASVGDRVTITCRASH |
| | YTFRSYGISWVRQAPGQGLEWMGWVA | SIDSWLAWYQQKPGKAPKLLIYKASYL |
| | PYSGNTNYAQKLQGRVTMTTDTSTST | ESGVPSRFSGSGSGTEFTLTISSLQPD |
| | AYMELRSLRSDDTAVYYCARDAGTYS | DFATYYCQLFQSLPPFTFGGGTKVEIK |
| | PYGYGMDVWGQGTTVTVSS(SEQ ID | (SEQ ID NO: 266) |
| | NO: 265) | |
| 25G9 | QVQLVQSGAEVKKPGASVKVSCKASG | DIQMTQSPSTLSASVGDRVTITCQASQ |
| | YTFRSYGISWVRQAPGQGLEWMGWVA | SIDSWLAWYQQKPGKAPKLLIYSASYL |
| | PYSGNTNYAQKLQGRVTMTTDTSTST | ESGVPSRFSGSGSGTEFTLTISSLQPD |
| | AYMELRSLRSDDTAVYYCARDAGTYS | DFATYYCQRFQSLPPFTFGGGTKVEIK |
| | PYGYGMDVWGQGTTVTVSS(SEQ ID | (SEQ ID NO: 304) |
| | NO: 303) | |
| 29D | QVQLVESGGGVVQPGRSLRLSCAASG | DIVMTQSPDSLAVSLGERATINCKSSQ |
| | FTFHSRGMHWVRQAPGKGLEWVAVIT | SVLFSSNNKNYLAWYQQKPGQPPKLLI |
| | YDGINKYYADSVEGRFTISRDNSKNT | YWASTRESGVPDRFSGSGSGTDFTLTI |
| | LYLQMNSLRAEDTAVYYCARDGVYYG | SSLQAEDVAVYYCQQFHSYPLTFGGGT |
| | VYDYWGQGTLVTVSS(SEQ ID NO: | KVEIK(SEQ ID NO: 342) |
| | 341) | |
| 29E | QVQLVESGGGVVQPGRSLRLSCAASG | DIVMTQSPDSLAVSLGERATINCKSSQ |
| | FTFRSYGMHWVRQAPGKGLEWVAVIT | SVLFSSNNKNYLAWYQQKPGQPPKLLI |
| | YDGINKYYADSVEGRFTISRDNSKNT | YWASTRESGVPDRFSGSGSGTDFTLTI |
| | LYLQMNSLRAEDTAVYYCARDGVYYG | SSLQAEDVAVYYCQQFHSYPLTFGGGT |
| | VYDYWGQGTLVTVSS(SEQ ID | KVEIK(SEQ ID NO: 380) |
| | NO: 379) | |
| 39A | QVQLVQSGAEVKKPGSSVKVSCKASG | EIVMTQSPATLSVSPGERATLSCRASQ |
| | GTFSSNAIGWVRQAPGQGLEWMGSII | SVSSNLAWYQQKPGQAPRLLIYGASTR |
| | PIIGFANYAQKFQGRVTITADESTST | ATGIPARFSGSGSGTEFTLTISSLQSE |
| | AYMELSSLRSEDTAVYYCARDSGYYY | DFAVYYCEQYNNLPLTFGGGTKVEIK |
| | GASSFGMDVWGQGTTVTVSS(SEQ | (SEQ ID NO: 418) |
| | ID NO: 417) | |
| 43B | QVQLQESGPGLVKPSQTLSLTCTVSG | EIVLTQSPGTLSLSPGERATLSCRASQ |
| | GSISSGQYWSWIRQHPGKGLEWIGEI | SVSSSYLAWYQQKPGQAPRLLIYGASS |
| | YYSGSTRYNPSLKSRVTISVDTSKNQ | RATGIPDRFSGSGSGTDFTLTISRLEP |
| | FSLKLSSVTAADTAVYYCARDAPYYY | EDFAVYYCQQVGVVPYTFGGGTKVEIK |
| | GGGYYYYMDVWGKGTTVTVSS(SEQ | (SEQ ID NO: 456) |
| | ID NO: 455) | |
| 43B1 | QVQLQESGPGLVKPSQTLSLTCTVSG | EIVLTQSPGTLSLSPGERATLSCRASE |
| | GSISSGQYWSWIRQHPGKGLEWIGEI | SVDSSYLAWYQQKPGQAPRLLIYGAST |
| | YYSGSTRYNPSLKSRVTISVDTSKNQ | RQTGIPDRFSGSGSGTDFTLTISRLEP |
| | FSLKLSSVTAADTAVYYCARDAPYYY | EDFAVYYCQQAGVVPYTFGGGTKVEIK |
| | GGGYYYYMDVWGKGTTVTVSS(SEQ | (SEQ ID NO: 494) |
| | ID NO: 493) | |
| 43B7 | QVQLQESGPGLVKPSQTLSLTCTVSG | EIVLTQSPGTLSLSPGERATLSCRASE |
| | GSISSGQYWSWIRQHPGKGLEWIGEI | SVDSSYLAWYQQKPGQAPRLLIYGADS |
| | YYSGSTRYNPSLKSRVTISVDTSKNQ | RATGIPDRFSGSGSGTDFTLTISRLEP |
| | FSLKLSSVTAADTAVYYCARDAPYYY | EDFAVYYCQQDGVVPYTFGGGTKVEIK |
| | GGGYYYYMDVWGKGTTVTVSS(SEQ | (SEQ ID NO: 532) |
| | ID NO: 531) | |
| 43D | QVQLQQWGAGLLKPSETLSLTCAVYG | EIVLTQSPGTLSLSPGERATLSCRASQ |
| | GSLSGYYWSWIRQPPGKGLEWIGEIG | SVSSSYLAWYQQKPGQAPRLLIYGASS |
| | ASGSTRYNPSLKSRVTISVDTSKNQF | RATGIPDRFSGSGSGTDFTLTISRLEP |
| | SLKLSSVTAADTAVYYCARDTPYYYE | EDFAVYYCQQVGVVPYTFGGGTKVEIK |
| | GGYYYYMDVWGKGTTVTVSS(SEQ | (SEQ ID NO: 570) |
| | ID NO: 569) | |
| 43D7 | QVQLQQWGAGLLKPSETLSLTCAVYG | EIVLTQSPGTLSLSPGERATLSCRASD |
| | GSLSGYYWSWIRQPPGKGLEWIGEIG | SVDSSYLAWYQQKPGQAPRLLIYGAFS |
| | ASGSTRYNPSLKSRVTISVDTSKNQF | RANGIPDRFSGSGSGTDFTLTISRLEP |
| | SLKLSSVTAADTAVYYCARDTPYYYE | EDFAVYYCQQAGVVPYTFGGGTKVEIK |
| | GGYYYYMDVWGKGTTVTVSS(SEQ | (SEQ ID NO: 608) |
| | ID NO: 607) | |
| 43D8 | QVQLQQWGAGLLKPSETLSLTCAVYG | EIVLTQSPGTLSLSPGERATLSCRASQ |
| | GSLSGYYWSWIRQPPGKGLEWIGEIG | SVSSSFLAWYQQKPGQAPRLLIYGAYS |
| | ASGSTRYNPSLKSRVTISVDTSKNQF | RATGIPDRFSGSGSGTDFTLTISRLEP |
| | SLKLSSVTAADTAVYYCARDTPYYYE | EDFAVYYCQQAGVVPYTFGGGTKVEIK |
| | GGYYYYMDVWGKGTTVTVSS(SEQ | (SEQ ID NO: 646) |
| | ID NO: 645) | |
| 43E | QVQLQESGPGLVKPSQTLSLTCTVSG | EIVLTQSPGTLSLSPGERATLSCRASQ |
| | GSISSGQYWSWIRQHPGKGLEWIGEI | SVSSSYLAWYQQKPGQAPRLLIYGASS |
| | YYSGSTRYNPSLKSRVTISVDTSKDQ | RATGIPDRFSGSGSGTDFTLTISRLEP |
| | FSLKLSSVTAADTAVYYCARDTPYYY | EDFAVYYCQQVGVVPYTFGGGTKVEIK |
| | DGGYYYYMDVWGKGTTVTVSS(SEQ | (SEQ ID NO: 684) |
| | ID NO: 683) | |
| 43Ea | QVQLQESGPGLVKPSQTLSLTCTVSG | EIVLTQSPGTLSLSPGERATLSCRASQ |
| | GSISSGQYWSWIRQHPGKGLEWIGEI | SVSSSYLAWYQQKPGQAPRLLIYGASS |
| | YYSGSTRYNPSLKSRVTISVDTSKNQ | RATGIPDRFSGSGSGTDFTLTISRLEP |
| | FSLKLSSVTAADTAVYYCARDTPYYY | EDFAVYYCQQVGVVPYTFGGGTKVEIK |
| | DGGYYYYMDVWGKGTTVTVSS(SEQ | (SEQ ID NO: 722) |
| | ID NO: 721) | |
| 54E | QVQLVQSGAEVKKPGASVKVSCKASG | DIQMTQSPSSLSASVGDRVTITCQASQ |
| | YTFANYYMHWVRQAPGQGLEWMGIIN | DISNSLNWYQQKPGKAPKLLIYDASNL |
| | PSGGITVYAQKFQGRVTMTRDTSTST | ETGVPSRFSGSRSGTDFTFTISSLQPE |
| | VYMELSSLRSEDTAVYYCARGGSKVA | DIATYYCQQYNFHPLTFGGGTKVEIK |
| | ALAFDIWGQGTMVTVSS(SEQ ID | (SEQ ID NO: 760) |
| | NO: 759) | |

TABLE 14
Variable region sequence consensus

| | VH Domain Consensus | VL Domain Consensus |
|---|---|---|
| Group | (SEQ ID NO) | (SEQ ID NO) |
| 1 | EVQLLESGGGLVQPGGSLR | DIQMTQSPSTLSASVGDRV |
| | LSCAASGFTFSx[D/S]YA | TITCRASQSISSWLAWYQQ |
| | Mx[A/G]WVRQAPGKGLEW | KPGKAPKLLIYKASSLESG |
| | VSx[A/T]ISGSGGLTYYA | VPSRFSGSGSGTEFTLTIS |
| | DSVKGRFTISRDNSKNTLY | SLQPDDFATYYCQQYKSYI |
| | LQMNSLRAEDTAVYYCAKA | TFGGGTKVEIK |
| | PYGYYMDVWGKGTTVTVSS | (SEQ ID NO: 762) |
| | (SEQ ID NO: 761) | |
| 25 | QVQLVQSGAEVKKPGASVK | DIQMTQSPSTLSASVGDRV |
| | VSCKASGYTFx[D/R]x[S/ | TITCx[R/Q]ASx[Q/E/ |
| | V/A]YGISWVRQAPGQG | H]SIx[S/D/N]x[S/N]W |
| | LEWMGWx[I/V]APYx[S/ | LAWYQQKPGKAPKLLIYx |
| | N]GNTNYAQKLQGRVTMTT | [K/S]Ax[S/Y]x[S/Y/N] |
| | DTSTSTAYMELRSLRSDDT | LEx[S/Y]GVPSRFSGSGS |
| | AVYYCARDAGTYSPx[F/ | GTEFTLTISSLQPDDFATY |
| | Y]GYGMDVWGQGTTVTVSS | YCQx[Q/L/R]FQx[S/K] |
| | (SEQ ID NO: 763) | LPPFTFGGGTKVEIK |
| | | (SEQ ID NO: 764) |
| 29 | QVQLVESGGGVVQPGRSLR | DIVMTQSPDSLAVSLGERA |
| | LSCAASGFTFx[H/R]Sx | TINCKSSQSVLFSSNNKNY |
| | [R/Y]GMHWVRQAPGKGLEW | LAWYQQKPGQPPKLLIYWA |
| | VAVITYDGINKYYADSVEG | STRESGVPDRFSGSGSGTD |
| | RFTISRDNSKNTLYLQMNS | FTLTISSLQAEDVAVYYCQ |
| | LRAEDTAVYYCARDGVYYG | QFHSYPLTFGGGTKVEIK |
| | VYDYWGQGTLVTVSS | (SEQ ID NO: 766) |
| | (SEQ ID NO: 765) | |
| 39 | QVQLVQSGAEVKKPGSSVK | EIVMTQSPATLSVSPGERA |
| | VSCKASGGTFSSNAIGWVR | TLSCRASQSVSSNLAWYQQ |
| | QAPGQGLEWMGSIIPIIGF | KPGQAPRLLIYGASTRATG |
| | ANYAQKFQGRVTITADEST | IPARFSGSGSGTEFTLTIS |
| | STAYMELSSLRSEDTAVYY | SLQSEDFAVYYCEQYNNLP |
| | CARDSGYYYGASSFGMDVW | LTFGGGTKVEIK |
| | GQGTTVTVSS | (SEQ ID NO: 768) |
| | (SEQ ID NO: 767) | |
| 43 | QVQLQx[E/Q]x[S/W]Gx | EIVLTQSPGTLSLSPGERA |
| | [P/A]GLx[V/L]KPSx[Q/ | TLSCRASx[Q/E/D]SVx |
| | E]TLSLTCx[T/A]Vx[S/ | [S/D]SSx[Y/F]LAWYQQK |
| | Y]GGSx[I/L]SSGx[Q/ | PGQAPRLLIYGAx[S/D/ |
| | Y]YWSWIRQx[H/P]PG | F/Y]x[S/T]Rx[A/Q]x |
| | KGLEWIGEIx[Y/G]x[Y/ | [T/N]GIPDRFSGSGSGTDF |
| | A]SGSTRYNPSLKSRVTIS | TLTISRLEPEDFAVYYCQQ |
| | VDTSKx[N/D]QFSLKLSS | x[V/A/D]GVVPYTFGGGT |
| | VTAADTAVYYCARDx[T/ | KVEIK |
| | A]PYYYx[E/G/D]GGYYY | (SEQ ID NO: 770) |
| | YMDVWGKGTTVTVSS | |
| | (SEQ ID NO: 769) | |
| 54 | QVQLVQSGAEVKKPGASVK | DIQMTQSPSSLSASVGDRV |
| | VSCKASGYTFANYYMHWVR | TITCQASQDISNSLNWYQQ |
| | QAPGQGLEWMGHNPSGGIT | KPGKAPKLLIYDASNLETG |
| | VYAQKFQGRVTMTRDTSTS | VPSRFSGSRSGTDFTFTIS |
| | TVYMELSSLRSEDTAVYYC | SLQPEDIATYYCQQYNFHP |
| | ARGGSKVAALAFDIWGQGT | LTFGGGTKVEIK |
| | MVTVSS | (SEQ ID NO: 772) |
| | (SEQ ID NO: 771) | |

TABLE 15
Antibody 1F-CDR Sequences

| | | Exemplary* | Kabat | Chothia | AbM | Contact | IMGT |
|---|---|---|---|---|---|---|---|
| VH | VH | GFTFSDYAMG | DYAMG (SEQ | GFTFSDY | GFTFSDYAMG | SDYAMG | GFTFSDYA |
| CDR | CDR1 | (SEQ ID | ID NO: 7) | (SEQ ID | (SEQ ID | (SEQ ID | (SEQ ID |
| Seq. | | NO: 1) | | NO: 13) | NO: 19) | NO: 25) | NO: 3l) |
| | VH | TISGSGGLTY | TISGSGGLTYY | GSGG | TISGSGGLTY | WVSTISG | ISGSGGLT |
| | CDR2 | YADSVKG | ADSVKG | (SEQ ID | (SEQ ID | SGGLTY | (SEQ ID |
| | | (SEQ ID | (SEQ ID NO: | NO: 14) | NO: 20) | (SEQ ID | NO: 32) |
| | | NO: 2) | 8) | | | NO: 26) | |
| | VH | APYGYYMDV | APYGYYMDV | PYGYYMD | APYGYYMDV | AKAPYGY | AKAPYGYY |
| | CDR3 | (SEQ ID NO: | (SEQ ID NO: | (SEQ ID | (SEQ ID | YMD | MDV (SEQ |
| | | 3) | 9) | NO: 15) | NO: 21) | (SEQ ID | ID NO: |
| | | | | | | NO: 27) | 33) |
| VL | VL | RASQSISSWLA | RASQSISSWLA | SQSISSW | RASQSISSWL | SSWLAWY | QSISSW |
| CDR | CDR1 | (SEQ ID NO: | (SEQ ID NO: | (SEQ ID | A (SEQ ID | (SEQ ID | (SEQ ID |
| Seq. | | 4) | 10) | NO: 16) | NO: 22) | NO: 28) | NO: 34) |
| | VL | KASSLES | KASSLES | KAS | KASSLES | LLIYKAS | KAS |
| | CDR2 | (SEQ ID NO: | (SEQ ID NO: | | (SEQ ID | SLE | |
| | | 5) | 11) | | NO: 23) | (SEQ ID | |
| | | | | | | NO: 29) | |
| | VL | QQYKSYIT | QQYKSYIT) | YKSYI | QQYKSYIT | QQYKSYI | QQYKSYIT |
| | CDR3 | (SEQ ID | (SEQ ID NO: | (SEQ ID | (SEQ ID | (SEQ ID | (SEQ ID |
| | | NO: 6) | 12) | NO: 18) | NO: 24) | NO: 30) | NO: 36) |

VH Sequence*:
EVQLLESGGGLVQPGGSLRLSCAASGFTFSDYAMGWVRQAPGKGLEWVSTISGSGGLTYYADSVKGR
FTISRDNSKNTLYLQMNSLRAEDTAVYYCAKAPYGYYMDVWGKGTTVTVSS (SEQ ID NO: 37)
VL Sequence*:
DIQMTQSPSTLSASVGDRVTITCRASQSISSWLAWYQQKPGKAPKWYKASSLESGVPSRFSGSGSGT
EFTLTISSLQPDDFATYYCQQYKSYITFGGGTKVEIK (SEQ ID NO: 38)
*Exemplary CDR sequences encompass amino acids as determined by Kabat plus Chothia

TABLE 16
Antibody 1G-CDR Sequences

| | | Exemplary* | Kabat | Chothia | AbM | Contact | IMGT |
|---|---|---|---|---|---|---|---|
| VH | VH | GFTFSSYAMA | SYAMA (SEQ | GFTFSSY | GFTFSSYAMA | SSYAMA (SEQ | GFTFSSYA |
| CDR | CDR1 | (SEQ ID | ID NO: 45) | (SEQ ID | (SEQ ID | ID NO: 63) | (SEQ ID |
| Seq. | | NO: 39) | | NO: 51) | NO: 57) | | NO: 69) |
| | VH | AISGSGGLTY | AISGSGGLTY | GSGG | AISGSGGLTY | WVSAISGSGGL | ISGSGGLT |
| | CDR2 | YADSVKG | YADSVKG | (SEQ ID | (SEQ ID | TY (SEQ ID | (SEQ ID |
| | | (SEQ ID | (SEQ ID | NO: 52) | NO: 58) | NO: 64) | NO: 70) |
| | | NO: 40) | NO: 46) | | | | |
| | VH | APYGYYMDV | APYGYYMDV | PYGYYMD | APYGYYMDV | AKAPYGYYMD | AKAPYGYY |
| | CDR3 | (SEQ ID | (SEQ ID | (SEQ ID | (SEQ ID | (SEQ ID NO: | MDV (SEQ |
| | | NO: 41) | NO: 47) | NO: 53) | NO: 59) | 65) | ID NO: |
| | | | | | | | 71) |
| VL | VL | RASQSISSWL | RASQSISSWL | SQSISSW | RASQSISSWL | SSWLAWY | QSISSW |
| CDR | CDR1 | A (SEQ ID | A (SEQ ID | (SEQ ID | A (SEQ ID | (SEQ ID NO: | (SEQ ID |
| Seq. | | NO: 42) | NO: 48) | NO: 54) | NO: 60) | 66) | NO: 72) |
| | VL | KASSLES | KASSLES | KAS | KASSLES | LLIYKASSLE | KAS |
| | CDR2 | (SEQ ID | (SEQ ID | | (SEQ ID | (SEQ ID NO: | |
| | | NO: 43) | NO: 49) | | NO: 61) | 67) | |
| | VL | QQYKSYIT | QQYKSYIT | YKSYI | QQYKSYIT | QQYKSYI | QQYKSYIT |
| | CDR3 | (SEQ ID | (SEQ ID | (SEQ ID | (SEQ ID | (SEQ ID NO: | (SEQ ID |
| | | NO: 44) | NO: 50) | NO: 56) | NO: 62) | 68) | NO: 74) |

VH Sequence*:
EVQLLESGGGLVQPGGSLRLSCAASGFTFSSYAMAWVRQAPGKGLEWVSAISGSGGLTYYADSVKGRF
TISRDNSKNTLYLQMNSLRAEDTAVYYCAKAPYGYYMDVWGKGTTVTVSS (SEQ ID NO: 75)
VL Sequence*:
DIQMTQSPSTLSASVGDRVTITCRASQSISSWLAWYQQKPGKAPKLLIYKASSLESGVPSRFSGSGSG
TEFTLTISSLQPDDFATYYCQQYKSYITFGGGTKVEIK (SEQ ID NO: 76)
*Exemplary CDR sequences encompass amino acids as determined by Kabat plus Chothia

TABLE 17
Antibody 25A-CDR Sequences

| | | Exemplary* | Kabat | Chothia | AbM | Contact | IMGT |
|---|---|---|---|---|---|---|---|
| VH | VH | GYTFDVYGIS | VYGIS (SEQ | GYTFDVY | GYTFDVYGIS | DVYGIS | GYTFDVYG |
| CDR | CDR1 | (SEQ ID NO: | ID NO: 83) | (SEQ ID | (SEQ ID NO: | (SEQ ID | (SEQ ID |
| Seq. | | 77) | | NO: 89) | 95) | NO: 101) | NO: 107) |
| | VH | WIAPYNGNTNY | WIAPYNGNTNY | PYNG | WIAPYNGNTN | WMGWIAPY | IAPYNGNT |
| | CDR2 | AQKLQG (SEQ | AQKLQG (SEQ | (SEQ ID | (SEQ ID NO: | NGNTN | (SEQ ID |
| | | ID NO: 78) | ID NO: 84) | NO: 90) | 96) | (SEQ ID | NO: 108) |
| | | | | | | NO: 102) | |
| | VH | DAGTYSPFGYG | DAGTYSPFGYG | AGTYSPF | DAGTYSPFGYG | ARDAGTYS | ARDAGTYS |
| | CDR3 | MDV (SEQ ID | MDV (SEQ ID | GYGMD | MDV (SEQ ID | PFGYGMD | PFGTGMDV |
| | | NO: 79) | NO: 85) | (SEQ ID | NO: 97) | (SEQ ID | (SEQ ID |
| | | | | NO: 91) | | NO: 103) | NO: 109) |
| VL | VL | RASQSISSWLA | RASQSISSWLA | SQSISSW | RASQSISSWLA | SSWLAWY | QSISSW |
| CDR | CDR1 | (SEQ ID NO: | (SEQ ID NO: | (SEQ ID | (SEQ ID NO: | (SEQ ID | (SEQ ID |
| Seq. | | 80) | 86) | NO: 92) | 98) | NO: 104) | NO: 110) |
| | VL | KASSLES | KASSLES | KAS | KASSLES | LLIYKASS | KAS |
| | CDR2 | (SEQ ID NO: | (SEQ ID NO: | | (SEQ ID NO: | LE (SEQ | |
| | | 81) | 87) | | 99) | ID NO: | |
| | | | | | | 105) | |
| | VL | QQFQSLPPFT | QQFQSLPPFT | FQSLPPF | QQFQSLPPFT | QQFQSLPP | QQFQSLPP |
| | CDR3 | (SEQ ID NO: | (SEQ ID NO: | (SEQ ID | (SEQ ID NO: | F (SEQ | FT (SEQ |
| | | 82) | 88) | NO: 94) | 100) | ID NO: | ID NO: |
| | | | | | | 106) | 112) |

VH Sequence*:
QVQLVQSGAEVKKPGASVKVSCKASGYTFDVYGISWVRQAPGQGLEWMGWIAPYNGNTNYAQKLQGRVTMT
TDTSTSTAYMELRSLRSDDTAVYYCARDAGTYSPFGYGMDVWGQGTTVTVSS (SEQ ID NO: 113)
VL Sequence*:
DIQMTQSPSTLSASVGDRVTITCRASQSISSWLAWYQQKPGKAPKWYKASSLESGVPSRFSGSGSGTEFTL
TISSLQPDDFATYYCQQFQSLPPFTFGGGTKVEIK (SEQ ID NO: 114)
*Exemplary CDR sequences encompass amino acids as determined by Kabat plus Chothia

TABLE 18
Antibody 25A3-CDR Sequences

| | | Exemplary* | Kabat | Chothia | AbM | Contact | IMGT |
|---|---|---|---|---|---|---|---|
| VH | VH | GYTFDVYGIS | VYGIS (SEQ | GYTFDVY | GYTFDVYGIS | DVYGIS (SEQ | GYTFDVYG |
| CDR | CDR1 | (SEQ ID NO: | ID NO: 121) | (SEQ ID | (SEQ ID NO: | ID NO: 139) | (SEQ ID |
| Seq. | | 115) | | NO: 127) | 133) | | NO: 145) |
| | VH | WIAPYSGNTNY | WIAPYSGNTNY | PYSG | WIAPYSGNTN | WMGWIAPYSGN | IAPYSGNT |
| | CDR2 | AQKLQG (SEQ | AQKLQG (SEQ | (SEQ ID | (SEQ ID NO: | TN (SEQ ID | (SEQ ID |
| | | ID NO: 116) | ID NO: 122) | NO: 128) | 134) | NO: 140) | NO: 146) |
| | VH | DAGTYSPFGYG | DAGTYSPFGYG | AGTYSPFG | DAGTYSPFGYG | ARDAGTYSPFG | ARDACTYS |
| | CDR3 | MDV (SEQ ID | MDV (SEQ ID | YGMD | MDV (SEQ ID | YGMD (SEQ | PFGTGMDV |
| | | NO: 117) | NO: 123) | (SEQ ID | NO: 135) | ID NO: 141) | (SEQ ID |
| | | | | NO: 129) | | | NO: 147) |
| VL | VL | QASQSINNNWL | QASQSINNWLA | SQSINNW | QASQSINNWLA | NNWLAWY | QSINNW |
| CDR | CDR1 | A (SEQ ID | (SEQ ID NO: | (SEQ ID | (SEQ ID NO: | (SEQ ID NO: | (SEQ ID |
| Seq. | | NO: 118) | 124) | NO: 130) | 136) | 142) | NO: 148) |
| | VL | KAYNLES | KAYNLES | KAY | KAYNLES | LLIYKAYNLE | KAY |
| | CDR2 | (SEQ ID NO: | (SEQ ID NO: | | (SEQ ID NO: | (SEQ ID NO: | |
| | | 119) | 125) | | 137) | 143) | |
| | VL | QLFQSLPPFT | QLFQSLPPFT | FQSLPPF | QLFQSLPPFT | QLFQSLPPF | QLFQSLPP |
| | CDR3 | (SEQ ID NO: | (SEQ ID NO: | (SEQ ID | (SEQ ID NO: | (SEQ ID NO: | FT (SEQ |
| | | 120) | 126) | NO: 132) | 138) | 144) | ID NO: |
| | | | | | | | 150) |

VH Sequence*:
QVQLVQSGAEVKKPGASVKVSCKASGYTFDVYGISWVRQAPGQGLEWMGWIAPYSGNTNYAQKLQGRVTM
TTDTSTSTAYMELRSLRSDDTAVYYCARDAGTYSPFGYGMDVWGQGTTVTVSS (SEQ ID NO: 151)
VL Sequence*:
DIQMTQSPSTLSASVGDRVTITCQASQSINNWLAWYQQKPGKAPKLLIYKAYNLESGVPSRFSGSGSGTE
FTLTISSLQPDDFATYYCQLFQSLPPFTFGGGTKVEIK (SEQ ID NO: 152)
*Exemplary CDR sequences encompass amino acids as determined by Kabat plus Chothia

TABLE 19a
Antibody 25A5-CDR Sequences

| | | Exemplary* | Kabat | Chothia | AbM | Contact | IMGT |
|---|---|---|---|---|---|---|---|
| VH | VH | GYTFDVYGIS | VYGIS (SEQ | GYTFDVY | GYTFDVYGIS | DVYGIS | GYTFDVYG |
| CDR | CDR1 | (SEQ ID NO: | ID NO: 159) | (SEQ ID | (SEQ ID NO: | (SEQ ID | (SEQ ID |
| Seq. | | 153) | | NO: 165) | 171) | NO: 177) | NO: 183) |
| | VH | WIAPYSGNTNY | WIAPYSGNTNY | PYSG | WIAPYSGNTN | WMGWIAPYSGN | IAPYSGNT |
| | CDR2 | AQKLQG (SEQ | AQKLQG (SEQ | (SEQ ID | (SEQ ID NO: | TN (SEQ ID | (SEQ ID |
| | | ID NO: 154) | ID NO: 160) | NO: 166) | 172) | NO: 178) | NO: 184) |
| | VH | DAGTYSPFGYG | DAGTYSPFGYG | AGTYSPFG | DAGTYSPFGYG | ARDAGTYSPFG | ARDACTYS |
| | CDR3 | MDV (SEQ ID | MDV (SEQ ID | YGMD | MDV (SEQ ID | YGMD (SEQ | PFGTGMDV |
| | | NO: 155) | NO: 161) | (SEQ ID | NO: 173) | ID NO: 179) | (SEQ ID |
| | | | | NO: 167) | | | NO: 185) |
| VL | VL | RASESISNWLA | RASESISNWLA | SESISNW | RASESISNWLA | SNWLAWY | ESISNW |
| CDR | CDR1 | (SEQ ID NO: | (SEQ ID NO: | (SEQ ID | (SEQ ID NO: | (SEQ ID NO: | (SEQ ID |
| Seq. | | 156) | 162) | NO: 168) | 174) | 180) | NO: 186) |
| | VL | KAYSLEY | KAYSLEY | KAY | KAYSLEY | LLIYKAYSLE | KAY |
| | CDR2 | (SEQ ID NO: | (SEQ ID NO: | | (SEQ ID NO: | (SEQ ID NO: | |
| | | 157) | 163) | | 175) | 181) | |
| | VL | QQFQKLPPFT | QQFQKLPPFT | FQKLPPF | QQFQKLPPFT | QQFQKLPPF | QQFQKLPP |
| | CDR3 | (SEQ ID NO: | (SEQ ID NO: | (SEQ ID | (SEQ ID NO: | (SEQ ID NO: | FT (SEQ |
| | | 158) | 164) | NO: 170) | 176) | 182) | ID NO: |
| | | | | | | | 188) |

VH Sequence*:
QVQLVQSGAEVKKPGASVKVSCKASGYTFDVYGISWVRQAPGQGLEWMGWIAPYSGNTNYAQKLQGRVTM
TTDTSTSTAYMELRSLRSDDTAVYYCARDAGTYSPFGYGMDVWGQGTTVTVSS (SEQ ID NO: 189)
VL Sequence*:
DIQMTQSPSTLSASVGDRVTITCRASESISNWLAWYQQKPGKAPKWYKAYSLEYGVPSRFSGSGSGTEFT
LTISSLQPDDFATYYCQQFQKLPPFTFGGGTKVEIK (SEQ ID NO: 190)
*Exemplary CDR sequences encompass amino acids as determined by Kabat plus Chothia

TABLE 19b
Antibody 25A5-T-CDR Sequences

| | | Exemplary* | Kabat | Chothia | AbM | Contact | IMGT |
|---|---|---|---|---|---|---|---|
| VH | VH | GYTFDAYGIS | AYGIS (SEQ | GYTFDAY | GYTFDAYGIS | DAYGIS (SEQ | GYTFDAYG |
| CDR | CDR1 | (SEQ ID NO: | ID NO: 890) | (SEQ ID | (SEQ ID NO: | ID NO: 908) | (SEQ ID |
| Seq. | | 884) | | NO: 896) | 902) | | NO: 914) |
| | VH | WIAPYSGNTNY | WIAPYSGNTNY | PYSG (SEQ | WIAPYSGNTN | WMGWIAPYSGN | IAPYSGNT |
| | CDR2 | AQKLQG (SEQ | AQKLQG (SEQ | ID NO: | (SEQ ID NO: | TN (SEQ ID | (SEQ ID |
| | | ID NO: 885) | ID NO: 891) | 897) | 903) | NO: 909) | NO: 915) |
| | VH | DAGTYSPFGYG | DAGTYSPFGYG | AGTYSPFGY | DAGTYSPFGYG | ARDAGTYSPFG | ARDACTYS |
| | CDR3 | MDV (SEQ ID | MDV (SEQ ID | GMD (SEQ | MDV (SEQ ID | YGMD (SEQ | PFGTGMDV |
| | | NO: 886) | NO: 892) | ID NO: | NO: 904) | ID NO: 910) | (SEQ ID |
| | | | | 898) | | | NO: 916) |
| VL | VL | RASESISNWLA | RASESISNWLA | SESISNW | RASESISNWLA | SNWLAWT | ESISNW |
| CDR | CDR1 | (SEQ ID NO: | (SEQ ID NO: | (SEQ ID | (SEQ ID NO: | (SEQ ID NO: | (SEQ ID |
| Seq. | | 887) | 893) | NO: 899) | 905) | 911) | NO: 917) |
| | VL | KAYSLEY | KAYSLEY | KAY | KAYSLEY | LLIYKAYSLE | KAY |
| | CDR2 | (SEQ ID NO: | (SEQ ID NO: | | (SEQ ID NO: | (SEQ ID NO: | |
| | | 888) | 894) | | 906) | 912) | |
| | VL | QQFQKLPPFT | QQFQKLPPFT | FQKLPPF | QQFQKLPPFT | QQFQKLPPF | QQFQKLPP |
| | CDR3 | (SEQ ID NO: | (SEQ ID NO: | (SEQ ID | (SEQ ID NO: | (SEQ ID NO: | FT (SEQ |
| | | 889) | 895) | NO: 901) | 907) | 913) | ID NO: |
| | | | | | | | 919) |

VH Sequence*:
QVQLVQSGAEVKKPGASVKVSCKASGYTFDAYGISWVRQAPGQGLEWMGWIAPYSGNTNYAQKLQGRVTM
TTDTSTSTAYMELRSLRSDDTAVYYCARDAGTYSPFGYGMDVWGQGTTVTVSS (SEQ ID NO: 836)
VL Sequence*:
DIQMTQSPSTLSASVGDRVTITCRASESISNWLAWYQQKPGKAPKWYKAYSLEYGVPSRFSGSGSGTEFT
LTISSLQPDDFATYYCQQFQKLPPFTFGGGTKVEIK (SEQ ID NO: 837)
*Exemplary CDR sequences encompass amino acids as determined by Kabat plus Chothia

TABLE 20
Antibody 25G-CDR Sequences

| | | Exemplary* | Kabat | Chothia | AbM | Contact | IMGT |
|---|---|---|---|---|---|---|---|
| VH | VH | GYTFRSYGIS | SYGIS | GYTFRSY | GYTFRSYGIS | RSYGIS | GYTFRSYG |
| CDR | CDR1 | (SEQ ID NO: | (SEQ ID NO: | (SEQ ID | (SEQ ID NO: | (SEQ ID | (SEQ ID |
| Seq. | | 191) | 197) | NO: 203) | 209) | NO: 215) | NO: 221) |
| | VH | WVAPYNGNTNY | WVAPYNGNTNY | PYNG | WVAPYNGNTN | WMGWVAPY | VAPYNGNT |
| | CDR2 | AQKLQG (SEQ | AQKLQG (SEQ | (SEQ ID | (SEQ ID NO: | NGNTN | (SEQ ID |
| | | ID NO: 192) | ID NO: 198) | NO: 204) | 210) | (SEQ ID | NO: 222) |
| | | | | | | NO: 216) | |
| | VH | DAGTYSPYGYG | DAGTYSPYGYG | AGTYSPYG | DAGTYSPYGYG | ARDAGTYS | ARDAGTYS |
| | CDR3 | MDV (SEQ ID | MDV (SEQ ID | YGMD | MDV (SEQ ID | PYGYGMD | PYGYGMDV |
| | | NO: 193) | NO: 199) | (SEQ ID | NO: 211) | (SEQ ID | (SEQ ID |
| | | | | NO: 205) | | NO: 217) | NO: 223) |
| VL | VL | RASQSISSWLA | RASQSISSWLA | SQSISSW | RASQSISSWLA | SSWLAWY | QSISSW |
| CDR | CDR1 | (SEQ ID NO: | (SEQ ID NO: | (SEQ ID | (SEQ ID NO: | (SEQ ID | (SEQ ID |
| Seq. | | 194) | 200) | NO: 206) | 212) | NO: 218) | NO: 224) |
| | VL | KASSLES | KASSLES | KAS | KASSLES | LLIYKASS | KAS |
| | CDR2 | (SEQ ID NO: | (SEQ ID NO: | | (SEQ ID NO: | LE (SEQ | |
| | | 195) | 201) | | 213) | ID NO: | |
| | | | | | | 219) | |
| | VL | QQFQSLPPFT | QQFQSLPPFT | FQSLPPF | QQFQSLPPFT | QQFQSLPP | QQFQSLPP |
| | CDR3 | (SEQ ID NO: | (SEQ ID NO: | (SEQ ID | (SEQ ID NO: | F (SEQ | FT (SEQ |
| | | 196) | 202) | NO: 208) | 214) | ID NO: | ID NO: |
| | | | | | | 220) | 226) |

VH Sequence*:
QVQLVQSGAEVKKPGASVKVSCKASGYTFRSYGISWVRQAPGQGLEWMGWVAPYNGNTNYAQKLQGRVTM
TTDTSTSTAYMELRSLRSDDTAVYYCARDAGTYSPYGYGMDVWGQGTTVTVSS (SEQ ID NO: 227)
VL Sequence*:
DIQMTQSPSTLSASVGDRVTITCRASQSISSWLAWYQQKPGKAPKWYKASSLESGVPSRFSGSGSGTEFT
LTISSLQPDDFATYYCQQFQSLPPFTFGGGTKVEIK (SEQ ID NO: 228)
*Exemplary CDR sequences encompass amino acids as determined by Kabat plus Chothia

TABLE 21
Antibody 25G1-CDR Sequences

| | | Exemplary* | Kabat | Chothia | AbM | Contact | IMGT |
|---|---|---|---|---|---|---|---|
| VH | VH | GYTFRSYGIS | SYGIS (SEQ | GYTFRSY | GYTFRSYGIS | RSYGIS | GYTFRSYG |
| CDR | CDR1 | (SEQ ID NO: | ID NO: 235) | (SEQ ID | (SEQ ID NO: | (SEQ ID | (SEQ ID |
| Seq. | | 229) | | NO 241) | 247) | NO: 253) | NO: 259) |
| | VH | WVAPYSGNTNY | WVAPYSGNTNY | PYSG (SEQ | WVAPYSGNTN | WMGWVAPY | VAPYSGNT |
| | CDR2 | AQKLQG (SEQ | AQKLQG (SEQ | ID NO: | (SEQ ID NO: | SGNTN | (SEQ ID |
| | | ID NO: 230) | ID NO: 236) | 242) | 248) | (SEQ ID | NO: 260) |
| | | | | | | NO: 254) | |
| | VH | DAGTYSPYGYG | DAGTYSPYGYG | AGTYSPYGY | DAGTYSPYGYG | ARDAGTYS | ARDAGTYS |
| | CDR3 | MDV (SEQ ID | MDV (SEQ ID | GMD (SEQ | MDV (SEQ ID | PYGYGMD | PYGYGMDV |
| | | NO: 231) | NO: 237) | ID NO: | NO: 240) | (SEQ ID | (SEQ ID |
| | | | | 243) | | NO: 255) | NO: 26l) |
| VL | VL | RASHSIDSWLA | RASHSIDSWLA | SHSIDSW | RASHSIDSWLA | DSWLAWY | HSIDSW |
| CDR | CDR1 | (SEQ ID NO: | (SEQ ID NO: | (SEQ ID | (SEQ ID NO: | (SEQ ID | (SEQ ID |
| Seq. | | 232) | 238) | NO: 244) | 250) | NO: 256) | NO: 262) |
| | VL | KASYLES | KASYLES | KAS | KASYLES | LLIYKASY | KAS |
| | CDR2 | (SEQ ID NO: | (SEQ ID NO: | | (SEQ ID NO: | LE (SEQ | |
| | | 233) | 239) | | 251) | ID NO: | |
| | | | | | | 257) | |
| | VL | QLFQSLPPFT | QLFQSLPPFT | FQSLPPF | QLFQSLPPFT | QLFQSLPP | QLFQSLPP |
| | CDR3 | (SEQ ID NO: | (SEQ ID NO: | (SEQ ID | (SEQ ID NO: | F (SEQ | FT (SEQ |
| | | 234) | 240) | NO: 246) | 252) | ID NO: | ID NO: |
| | | | | | | 258) | 264) |

VH Sequence*:
QVQLVQSGAEVKKPGASVKVSCKASGYTFRSYGISWVRQAPGQGLEWMGWVAPYSGNTNYAQKLQGRVTM
TTDTSTSTAYMELRSLRSDDTAVYYCARDAGTYSPYGYGMDVWGQGTTVTVSS (SEQ ID NO: 265)
VL Sequence*:
DIQMTQSPSTLSASVGDRVTITCRASHSIDSWLAWYQQKPGKAPKWYKASYLESGVPSRFSGSGSGTEFT
LTISSLQPDDFATYYCQLFQSLPPFTFGGGTKVEIK (SEQ ID NO: 266)
*Exemplary CDR sequences encompass amino acids as determined by Kabat plus Chothia

TABLE 22
Antibody 25G9-CDR Sequences

| | | Exemplary* | Kabat | Chothia | AbM | Contact | IMGT |
|---|---|---|---|---|---|---|---|
| VH | VH | GYTFRSYGIS | SYGIS (SEQ | GYTFRSY | GYTFRSYGIS | RSYGIS | GYTFRSYG |
| CDR | CDR1 | (SEQ ID NO: | ID NO: 273) | (SEQ ID | (SEQ ID NO: | (SEQ ID NO: | (SEQ ID |
| Seq. | | 267) | | NO: 279) | 285) | 291) | NO: 297) |
| | VH | WVAPYSGNTNY | WVAPYSGNTNY | PYSG | WVAPYSGNTN | WMGWVAPYSGN | VAPYSGNT |
| | CDR2 | AQKLQG (SEQ | AQKLQG (SEQ | (SEQ ID | (SEQ ID NO: | TN (SEQ ID | (SEQ ID |
| | | ID NO: 268) | ID NO: 274) | NO: 280) | 286) | NO: 292) | NO: 298) |
| | VH | DAGTYSPYGYG | DAGTYSPYGYG | AGTYSPYG | DAGTYSPYGYG | ARDAGTYSPYG | ARDAGTYS |
| | CDR3 | MDV (SEQ ID | MDV (SEQ ID | YGMD | MDV (SEQ ID | YGMD (SEQ | PYGYGMDV |
| | | NO: 269) | NO: 275) | (SEQ ID | NO: 287) | ID NO: 293) | (SEQ ID |
| | | | | NO: 281) | | | NO: 299) |
| VL | VL | QASQSIDSWLA | QASQSIDSWLA | SQSIDSW | QASQSIDSWLA | DSWLAWY | QSIDSW |
| CDR | CDR1 | (SEQ ID NO: | (SEQ ID NO: | (SEQ ID | (SEQ ID NO: | (SEQ ID NO: | (SEQ ID |
| Seq. | | 270) | 276) | NO: 282) | 288) | 294) | NO: 300) |
| | VL | SASYLES | SASYLES | SAS | SASYLES | LLIYSASYLE | SAS |
| | CDR2 | (SEQ ID NO: | (SEQ ID NO: | | (SEQ ID NO: | (SEQ ID NO: | |
| | | 27l) | 277) | | 289) | 295) | |
| | VL | QRFQSLPPFT | QRFQSLPPFT | FQSLPPF | QRFQSLPPFT | QRFQSLPPF | QRFQSLPP |
| | CDR3 | (SEQ ID NO: | (SEQ ID NO: | (SEQ ID | (SEQ ID NO: | (SEQ ID NO: | FT (SEQ |
| | | 272) | 278) | NO: 284) | 290) | 296) | ID NO: |
| | | | | | | | 302) |

VH Sequence*:
QVQLVQSGAEVKKPGASVKVSCKASGYTFRSYGISWVRQAPGQGLEWMGWVAPYSGNTNYAQKLQGRVTM
TTDTSTSTAYMELRSLRSDDTAVYYCARDAGTYSPYGYGMDVWGQGTTVTVSS (SEQ ID NO: 303)
VL Sequence*:
DIQMTQSPSTLSASVGDRVTITCQASQSIDSWLAWYQQKPGKAPKWYSASYLESGVPSRFSGSGSGTEFT
LTISSLQPDDFATYYCQRFQSLPPFTFGGGTKVEIK (SEQ ID NO: 304)
*Exemplary CDR sequences encompass amino acids as determined by Kabat plus Chothia

TABLE 23
Antibody 29D-CDR Sequences

| | | Exemplary* | Kabat | Chothia | AbM | Contact | IMGT |
|---|---|---|---|---|---|---|---|
| VH | VH | GFTFHSRGMH | SRGMH (SEQ | GFTFHSR | GFTFHSRGMH | HSRGMH (SEQ | GFTFHSRG |
| CDR | CDR1 | (SEQ ID NO: | ID NO: 3l1) | (SEQ ID | (SEQ ID NO: | ID NO: 329) | (SEQ ID NO: |
| Seq. | | 305) | | NO: 317) | 323) | | 335) |
| | VH | VITYDGINKYY | VITYDGINKYY | YDGI | VITYDGINKY | WVAVITYDGIN | ITYDGINK |
| | CDR2 | ADSVEG (SEQ | ADSVEG (SEQ | (SEQ ID | (SEQ ID NO: | KY (SEQ ID | (SEQ ID NO: |
| | | ID NO: 306) | ID NO: 312) | NO: 318) | 324) | NO: 330) | 336) |
| | VH | DGVYYGVYDY | DGVYYGVYDY | GVYYGVYD | DGVYYGVYDY | ARDGVYYGVYD | ARDGVYYGVYD |
| | CDR3 | (SEQ ID NO: | (SEQ ID NO: | (SEQ ID | (SEQ ID NO: | (SEQ ID NO: | Y (SEQ ID |
| | | 307) | 313) | NO: 319) | 325) | 33l) | NO: 337) |
| VL | VL | KSSQSVLFSSN | KSSQSVLFSSN | SQSVLFSS | KSSQSVLFSSN | LFSSNNKNYLA | QSVLFSSNNKN |
| CDR | CDR1 | NKNYLA (SEQ | NKNYLA (SEQ | NNKNY | NKNYLA (SEQ | WY (SEQ ID | Y (SEQ ID |
| Seq. | | ID NO: 308) | ID NO: 314) | (SEQ ID | ID NO: 326) | NO: 332) | NO: 338) |
| | | | | NO: 320) | | | |
| | VL | WASTRES | WASTRES | WAS | WASTRES | LLIYWASTRE | WAS |
| | CDR2 | (SEQ ID NO: | (SEQ ID NO: | | (SEQ ID NO: | (SEQ ID NO: | |
| | | 309) | 315) | | 327) | 333) | |
| | VL | QQFHSYPLT | QQFHSYPLT | FHSYPL | QQFHSYPLT | QQFHSYPL | QQFHSYPLT |
| | CDR3 | (SEQ ID NO: | (SEQ ID NO: | (SEQ ID | (SEQ ID NO: | (SEQ ID NO: | (SEQ ID NO: |
| | | 310) | 316) | NO: 322) | 328) | 334) | 340) |

VH Sequence*:
QVQLVESGGGVVQPGRSLRLSCAASGFTFHSRGMHWVRQAPGKGLEWVAVITYDGINKYYADSVEGRFTIS
RDNSKNTLYLQMNSLRAEDTAVYYCARDGVYYGVYDYWGQGTLVTVSS (SEQ ID NO: 341)
VL Sequence*:
DIVMTQSPDSLAVSLGERATINCKSSQSVLFSSNNKNYLAWYQQKPGQPPKWYWASTRESGVPDRFSGSGS
GTDFTLTISSLQAEDVAVYYCQQFHSYPLTFGGGTKVEIK (SEQ ID NO: 342)
*Exemplary CDR sequences encompass amino acids as determined by Kabat plus Chothia

TABLE 24
Antibody 29E-CDR Sequences

| | | Exemplary* | Kabat | Chothia | AbM | Contact | IMGT |
|---|---|---|---|---|---|---|---|
| VH | VH | GFTFRSYGMH | SYGMH (SEQ | GFTFRSY | GFTFRSYGMH | RSYGMH (SEQ | GFTFRSYG |
| CDR | CDR1 | (SEQ ID NO: | ID NO: 349) | (SEQ ID | (SEQ ID NO: | ID NO: 367) | (SEQ ID |
| Seq. | | 343) | | NO: 355) | 361) | | NO: 373) |
| | VH | VITYDGINKYY | VITYDGINKYY | YDGI | VITYDGINKY | WVAVITYDGIN | ITYDGINK |
| | CDR2 | ADSVEG (SEQ | ADSVEG (SEQ | (SEQ ID | (SEQ ID NO: | KY (SEQ ID | (SEQ ID |
| | | ID NO: 344) | ID NO: 350) | NO: 356) | 362) | NO: 368) | NO: 374) |
| | VH | DGVYYGVYDY | DGVYYGVYDY | GVYYGVYD | DGVYYGVYDY | ARDGVYYGVYD | ARDGVYYG |
| | CDR3 | (SEQ ID NO: | (SEQ ID NO: | (SEQ ID | (SEQ ID NO: | (SEQ ID NO: | VYDY |
| | | 345) | 351) | NO: 357) | 363) | 369) | (SEQ ID |
| | | | | | | | NO: 375) |
| VL | VL | KSSQSVLFSSN | KSSQSVLFSSN | SQSVLFSS | KSSQSVLFSSN | LFSSNNKNYLA | QSVLFSSN |
| CDR | CDR1 | NKNYLA (SEQ | NKNYLA (SEQ | NNKNY | NKNYLA (SEQ | WY (SEQ ID | NKNY |
| Seq. | | ID NO: 346) | ID NO: 352) | (SEQ ID | ID NO: 364) | NO: 370) | (SEQ ID |
| | | | | NO: 358) | | | NO: 376) |
| | VL | WASTRES | WASTRES | WAS | WASTRES | LLIYWASTRE | WAS |
| | CDR2 | (SEQ ID NO: | (SEQ ID NO: | | (SEQ ID NO: | (SEQ ID NO: | |
| | | 347) | 353) | | 365) | 37l) | |
| | VL | QQFHSYPLT | QQFHSYPLT | FHSYPL | QQFHSYPLT | QQFHSYPL | QQFHSYPL |
| | CDR3 | (SEQ ID NO: | (SEQ ID NO: | (SEQ ID | (SEQ ID NO: | (SEQ ID NO: | T (SEQ |
| | | 348) | 354) | NO: 360) | 366) | 372) | ID NO: |
| | | | | | | | 378) |

VH Sequence*:
QVQLVESGGGVVQPGRSLRLSCAASGFTFRSYGMHWVRQAPGKGLEWVAVITYDGINKYYADSVEGRF
TISRDNSKNTLYLQMNSLRAEDTAVYYCARDGVYYGVYDYWGQGTLVTVSS (SEQ ID NO: 379)
VL Sequence*:
DIVMTQSPDSLAVSLGERATINCKSSQSVLFSSNNICNYLAWYQQKPGQPPKWYWASTRESGVPDRFS
GSGSGTDFTLTISSLQAEDVAVYYCQQFHSYPLTFGGGTKVEIK (SEQ ID NO: 380)
*Exemplary CDR sequences encompass amino acids as determined by Kabat plus Chothia

TABLE 25
Antibody 39A-CDR Sequences

| | | Exemplary* | Kabat | Chothia | AbM | Contact | IMGT |
|---|---|---|---|---|---|---|---|
| VH | VH | GGTFSSNAIG | SNAIG (SEQ | GGTFSSN | GGTFSSNAIG | SSNAIG (SEQ | GGTFSSNA |
| CDR | CDR1 | (SEQ ID NO: | ID NO: 387) | (SEQ ID | (SEQ ID NO: | ID NO: 405) | (SEQ ID |
| Seq. | | 381) | | NO: 393) | 399) | | NO: 411) |
| | VH | SIIPIIGFANY | SIIPIIGFANY | PIIG | SIIPIIGFAN | WMGSIIPIIGF | IIPIIGFA |
| | CDR2 | AQKFQG (SEQ | AQKFQG (SEQ | (SEQ ID | (SEQ ID NO: | AN (SEQ ID | (SEQ ID |
| | | ID NO: 382) | ID NO: 388) | NO: 394) | 400) | NO: 406) | NO: 412) |
| | VH | DSGYYYGASSF | DSGYYYGASSF | SGYYYGAS | DSGYYYGASSF | ARDSGYYYGAS | ARDSGYYY |
| | CDR3 | GMDV (SEQ | GMDV (SEQ | SFGMD | GMDV (SEQ | SFGMD (SEQ | GASSFGMD |
| | | ID NO: 383) | ID NO: 389) | (SEQ ID | ID NO: 401) | ID NO: 407) | V (SEQ |
| | | | | NO: 395) | | | ID NO: |
| | | | | | | | 413) |
| VL | VL | RASQSVSSNLA | RASQSVSSNLA | SQSVSSN | RASQSVSSNLA | SSNLAWY | QSVSSN |
| CDR | CDR1 | (SEQ ID NO: | (SEQ ID NO: | (SEQ ID | (SEQ ID NO: | (SEQ ID NO: | (SEQ ID |
| Seq. | | 384) | 390) | NO: 396) | 402) | 408) | NO: 414) |
| | VL | GASTRAT | GASTRAT | GAS | GASTRAT | LLIYGASTRA | GAS |
| | CDR2 | (SEQ ID NO: | (SEQ ID NO: | | (SEQ ID NO: | (SEQ ID NO: | |
| | | 385) | 391) | | 403) | 409) | |
| | VL | EQYNNLPLT | EQYNNLPLT | YNNLPL | EQYNNLPLT | EQYNNLPL | EQYNNLPL |
| | CDR3 | (SEQ ID NO: | (SEQ ID NO: | (SEQ ID | (SEQ ID NO: | (SEQ ID NO: | T (SEQ |
| | | 386) | 392) | NO: 398) | 404) | 410) | ID NO: |
| | | | | | | | 416) |

VH Sequence*:
QVQLVQSGAEVKKPGSSVKVSCKASGGTFSSNAIGWVRQAPGQGLEWMGSIIPIIGFANYAQKFQGRVTIT
ADESTSTAYMELSSLRSEDTAVYYCARDSGYYYGASSFGMDVWGQGTTVTVSS (SEQ ID NO: 417)
VL Sequence*:
EIVMTQSPATLSVSPGERATLSCRASQSVSSNLAWYQQKPGQAPRLLIYGASTRATGIPARFSGSGSGTEF
TLTISSLQSEDFAVYYCEQYNNLPLTFGGGTKVEIK (SEQ ID NO: 418)
*Exemplary CDR sequences encompass amino acids as determined by Kabat plus Chothia

TABLE 26
Antibody 43B-CDR Sequences

| | | Exemplary* | Kabat | Chothia | AbM | Contact | IMGT |
|---|---|---|---|---|---|---|---|
| VH | VH | GGSISSGQYWS | SGQYWS (SEQ | GGSISSGQ | GGSISSGQYWS | SSGQYWS | GGSISSGQY |
| CDR | CDR1 | (SEQ ID NO: | ID NO: 425) | (SEQ ID | (SEQ ID NO: | (SEQ ID NO: | (SEQ ID |
| Seq. | | 419) | | NO: 431) | 437) | 443) | NO: 449) |
| | VH | EIYYSGSTRYN | EIYYSGSTRYN | YSG (SEQ | EIYYSGSTR | WIGEIYYSGST | IYYSGST |
| | CDR2 | PSLKS (SEQ | PSLKS (SEQ | ID NO: | (SEQ ID NO: | R (SEQ ID | (SEQ ID |
| | | ID NO: 420) | ID NO: 426) | 432) | 438) | NO: 444) | NO: 450) |
| | VH | DAPYYYGGGYY | DAPYYYGGGYY | APYYYGGG | DAPYYYGGGYY | ARDAPYYYGGG | ARDAPYYYG |
| | CDR3 | YYMDV (SEQ | YYMDV (SEQ | YYYYMD | YYMDV (SEQ | YYYYMD (SEQ | GGYYYYMDV |
| | | ID NO: 42l) | ID NO: 427) | (SEQ ID | ID NO: 439) | ID NO: 445) | (SEQ ID |
| | | | | NO: 433) | | | NO: 451) |
| VL | VL | RASQSVSSSYL | RASQSVSSSYL | SQSVSSSY | RASQSVSSSYL | SSSYLAWY | QSVSSSY |
| CDR | CDR1 | A (SEQ ID | A (SEQ ID | (SEQ ID | A (SEQ ID | (SEQ ID NO: | (SEQ ID |
| Seq. | | NO: 422) | NO: 428) | NO: 434) | NO: 440) | 446) | NO: 452) |
| | VL | GASSRAT | GASSRAT | GAS | GASSRAT | LLIYGASSRA | GAS |
| | CDR2 | (SEQ ID NO: | (SEQ ID NO: | | (SEQ ID | (SEQ ID NO: | |
| | | 423) | 429) | | NO: 441) | 447) | |
| | VL | QQVGVVPYT | QQVGVVPYT | VGVVPY | QQVGVVPYT | QQVGVVPY | QQVGVVPYT |
| | CDR3 | (SEQ ID NO: | (SEQ ID NO: | (SEQ ID | (SEQ ID NO: | (SEQ ID NO: | (SEQ ID |
| | | 424) | 430) | NO: 436) | 442) | 448) | NO: 454) |

VH Sequence*:
QVQLQESGPGLVKPSQTLSLTCTVSGGSISSGQYWSWIRQHPGKGLEWIGEIYYSGSTRYNPSLKSRVTIS
VDTSKNQFSLKLSSVTAADTAVYYCARDAPYYYGGGYYYYMDVWGKGTTVTVSS (SEQ ID NO: 455)
VL Sequence*:
EIVLTQSPGTLSLSPGERATLSCRASQSVSSSYLAWYQQKPGQAPRLLIYGASSRATGIPDRFSGSGSGTD
FTLTISRLEPEDFAVYYCQQVGVVPYTFGGGTKVEIK (SEQ ID NO: 456)
*Exemplary CDR sequences encompass amino acids as determined by Kabat & Chothia

TABLE 27
Antibody 43B1-CDR Sequences

| | | Exemplary* | Kabat | Chothia | AbM | Contact | IMGT |
|---|---|---|---|---|---|---|---|
| VH | VH | GGSISSGQYWS | SGQYWS (SEQ | GGSISSGQ | GGSISSGQYWS | SSGQYWS | GGSISSGQY |
| CDR | CDR1 | (SEQ ID NO: | ID NO: 463) | (SEQ ID | (SEQ ID NO: | (SEQ ID | (SEQ ID |
| Seq. | | 457) | | NO: 469) | 475) | NO: 481) | NO: 487) |
| | VH | EIYYSGSTRYN | EIYYSGSTRYN | YSG (SEQ | EIYYSGSTR | WIGEIYYS | IYYSGST |
| | CDR2 | PSLKS (SEQ | PSLKS (SEQ | ID NO: | (SEQ ID NO: | GSTR(SEQ | (SEQ ID |
| | | ID NO: 458) | ID NO: 464) | 470) | 476) | ID NO: | NO: 488) |
| | | | | | | 482) | |
| | VH | DAPYYYGGGYY | DAPYYYGGGYY | APYYYGGG | DAPYYYGGGYY | ARDAPYYY | ARDAPYYYG |
| | CDR3 | YYMDV (SEQ | YYMDV (SEQ | YYYYMD | YYMDV (SEQ | GGGYYYYM | GGYYYYMDV |
| | | ID NO: 459) | ID NO: 465) | (SEQ ID | ID NO: 477) | D (SEQ | (SEQ ID |
| | | | | NO: 471) | | ID NO: | NO: 489) |
| | | | | | | 483) | |
| VL | VL | RASESVDSSYL | RASESVDSSYL | SESVDSSY | RASESVDSSYL | DSSYLAWY | ESVDSSY |
| CDR | CDR1 | A (SEQ ID | A (SEQ ID | (SEQ ID | A (SEQ ID | (SEQ ID | (SEQ ID |
| Seq. | | NO: 460) | NO: 466) | NO: 472) | NO: 478) | NO: 484) | NO: 490) |
| | VL | GASTRQT | GASTRQT | GAS | GASTRQT | LLIYGAST | GAS |
| | CDR2 | (SEQ ID NO: | (SEQ ID NO: | | (SEQ ID NO: | RQ (SEQ | |
| | | 46l) | 467) | | 479) | ID NO: | |
| | | | | | | 485) | |
| | VL | QQAGVVPYT | QQAGVVPYT | AGVVPY | QQAGVVPYT | QQAGVVPY | QQAGVVPY |
| | CDR3 | (SEQ ID NO: | (SEQ ID NO: | (SEQ ID | (SEQ ID NO: | (SEQ ID | T (SEQ |
| | | 462) | 468) | NO: 474) | 480) | NO: 486) | ID NO: |
| | | | | | | | 492) |

VH Sequence*:
QVQLQESGPGLVKPSQTLSLTCTVSGGSISSGQYWSWIRQHPGKGLEWIGEIYYSGSTRYNPSLKSRVTIS
VDTSKNQFSLKLSSVTAADTAVYYCARDAPYYYGGGYYYYMDVWGKGTTVTVSS (SEQ ID NO: 493)
VL Sequence*:
EIVLTQSPGTLSLSPGERATLSCRASESVDSSYLAWYQQKPGQAPRLLIYGASTRQTGIPDRFSGSGSGTD
FTLTISRLEPEDFAVYYCQQAGVVPYTFGGGTKVEIK (SEQ ID NO: 494)
*Exemplary CDR sequences encompass amino acids as determined by Kabat & Chothia

TABLE 28
Antibody 43B7-CDR Sequences

| | | Exem- | | | | | |
|---|---|---|---|---|---|---|---|
| | | plary* | Kabat | Chothia | AbM | Contact | IMGT |
| VH | VH | GGSI | SGQY | GGSI | GGSI | SSGQ | GGSI |
| CDR | CDR1 | SSGQ | WS | SSGQ | SSGQ | YWS | SSGQ |
| Seq. | | YWS | (SEQ | (SEQ | YWS | (SEQ | Y |
| | | (SEQ | ID | ID | (SEQ | ID | (SEQ |
| | | ID | NO: | NO: | ID | NO: | ID |
| | | NO: | 501) | 507) | NO: | 519) | NO: |
| | | 495) | | | 513) | | 525) |
| | VH | EIYY | EIYY | YSG | EIYY | WIGE | IYYS |
| | CDR2 | SGST | SGST | (SEQ | SGST | IYYS | GST |
| | | RYNP | RYNP | ID | R | GSTR | (SEQ |
| | | SLKS | SLKS | NO: | (SEQ | (SEQ | ID |
| | | (SEQ | (SEQ | 508) | ID | ID | NO: |
| | | ID | ID | | NO: | NO: | 526) |
| | | NO: | NO: | | 514) | 520) | |
| | | 496) | 502) | | | | |
| | VH | DAPY | DAPY | APYY | DAPY | ARDA | ARDA |
| | CDR3 | YYGG | YYGG | YGGG | YYGG | PYYY | PYYY |
| | | GYY | GYY | YYY | GYY | GGG | GGG |
| | | YYMD | YYMD | YMD | YYMD | YYYY | YYYY |
| | | V | V | (SEQ | V | MD | MDV |
| | | (SEQ | (SEQ | ID | (SEQ | (SEQ | (SEQ |
| | | ID | ID | NO: | ID | ID | ID |
| | | NO: | NO: | 509) | NO: | NO: | NO: |
| | | 497) | 503) | | 515) | 521) | 527) |
| VL | VL | RASE | RASE | SESV | RASE | DSSY | ESVD |
| CDR | CDR1 | SVDS | SVDS | DSSY | SVDS | LAWY | SSY |
| Seq. | | SYLA | SYLA | (SEQ | SYLA | (SEQ | (SEQ |
| | | (SEQ | (SEQ | ID | (SEQ | ID | ID |
| | | ID | ID | NO: | ID | NO: | NO: |
| | | NO: | NO: | 510) | NO: | 522) | 528) |
| | | 498) | 504) | | 516) | | |
| | VL | GADS | GADS | GAD | GADS | LLIY | GAD |
| | CDR2 | RAT | RAT | (SEQ | RAT | GADS | (SEQ |
| | | (SEQ | (SEQ | ID | (SEQ | RA | ID |
| | | ID | ID | NO: | ID | (SEQ | NO: |
| | | NO: | NO: | 511) | NO: | ID | 529) |
| | | 499) | 505) | | 517) | NO: | |
| | | | | | | 523) | |
| | VL | QQDG | QQDG | DGVV | QQDG | QQDG | QQDG |
| | CDR3 | VVPY | VVPY | PY | VVPY | VVPY | VVPY |
| | | T | T | (SEQ | T | (SEQ | T |
| | | (SEQ | (SEQ | ID | (SEQ | ID | (SEQ |
| | | ID | ID | NO: | ID | NO: | ID |
| | | NO: | NO: | 512) | NO: | 524) | NO: |
| | | 500) | 506) | | 518) | | 530) |

VH Sequence*:
QVQLQESGPGLVKPSQTLSLTCTVSGGSISSGQYWSWIRQHPGKGLEWIG
EIYYSGSTRYNPSLKSRVTISVDTSKNQFSLKLSSVTAADTAVYYCARDA
PYYYGGGYYYYMDVWGKGTTVTVSS (SEQ ID NO: 531)
VL Sequence*:
EIVLTQSPGTLSLSPGERATLSCRASESVDSSYLAWYQQKPGQAPRLLIY
GADSRATGIPDRFSGSGSGTDFTLTISRLEPEDFAVYYCQQDGVVPYTFG
GGTKVEIK (SEQ ID NO: 532)
*Exemplary CDR sequences encompass amino acids as determined by Kabat & Chothia

TABLE 29
Antibody 43D-CDR Sequences

| | | Exem- | | | | | |
|---|---|---|---|---|---|---|---|
| | | plary* | Kabat | Chothia | AbM | Contact | IMGT |
| VH | VH | GGSL | GYYW | GGSL | GGSL | SGYY | GGSL |
| CDR | CDR1 | SGYY | S | SGY | SGYY | WS | SGYY |
| Seq. | | WS | (SEQ | (SEQ | WS | (SEQ | (SEQ |
| | | (SEQ | ID | ID | (SEQ | ID | ID |
| | | ID | NO: | NO: | ID | NO: | NO: |
| | | NO: | 539) | 545) | NO: | 557) | 563) |
| | | 533) | | | 551) | | |
| | VH | EIGA | EIGA | ASG | EIGA | WIGE | IGAS |
| | CDR2 | SGST | SGST | (SEQ | SGST | IGAS | GST |
| | | RYNP | RYNP | ID | R | GSTR | (SEQ |
| | | SLKS | SLKS | NO: | (SEQ | (SEQ | ID |
| | | (SEQ | (SEQ | 546) | ID | ID | NO: |
| | | ID | ID | | NO: | NO: | 564) |
| | | NO: | NO: | | 552) | 558) | |
| | | 534) | 540) | | | | |
| | VH | DTPY | DTPY | TPYY | DTPY | ARDT | ARDT |
| | CDR3 | YYEG | YYEG | YEGG | YYEG | PYYY | PYYY |
| | | GYYY | GYYY | YYY | GYYY | EGGY | EGG |
| | | YMDV | YMDV | YMD | YMDV | YYY | YYYY |
| | | (SEQ | (SEQ | (SEQ | (SEQ | MD | MDV |
| | | ID | ID | ID | ID | (SEQ | (SEQ |
| | | NO: | NO: | NO: | NO: | ID | ID |
| | | 535) | 541) | 547) | 553) | NO: | NO: |
| | | | | | | 559) | 565) |
| VL | VL | RASQ | RASQ | SQSV | RASQ | SSSY | QSVS |
| CDR | CDR1 | SVSS | SVSS | SSSY | SVSS | LAWY | SSY |
| Seq. | | SYLA | SYLA | (SEQ | SYLA | (SEQ | (SEQ |
| | | (SEQ | (SEQ | ID | (SEQ | ID | ID |
| | | ID | ID | NO: | ID | NO: | NO: |
| | | NO: | NO: | 548) | NO: | 560) | 566) |
| | | 536) | 542) | | 554) | | |
| | VL | GASS | GASS | GAS | GASS | LLIY | GAS |
| | CDR2 | RAT | RAT | (SEQ | RAT | GASS | (SEQ |
| | | (SEQ | (SEQ | ID | (SEQ | RA | ID |
| | | ID | ID | NO: | ID | (SEQ | NO: |
| | | NO: | NO: | 549) | NO: | ID | 567) |
| | | 537) | 543) | | 555) | NO: | |
| | | | | | | 561 | |
| | VL | QQVG | QQVG | VGVV | QQVG | QQVG | QQVG |
| | CDR3 | VVPY | VVPY | PY | VVPY | VVPY | VVPY |
| | | T | T | (SEQ | T | (SEQ | T |
| | | (SEQ | (SEQ | ID | (SEQ | ID | (SEQ |
| | | ID | ID | NO: | ID | NO: | ID |
| | | NO: | NO: | 550) | NO: | 562) | NO: |
| | | 538) | 544) | | 556) | | 568) |

VH Sequence*:
QVQLQQWGAGLLKPSETLSLTCAVYGGSLSGYYWSWIRQPPGKGLEWIGE
IGASGSTRYNPSLKSRVTISVDTSKNQFSLKLSSVTAADTAVYYCARDTP
YYYEGGYYYYMDVWGKGTTVTVSS (SEQ ID NO: 569)
VL Sequence*:
EIVLTQSPGTLSLSPGERATLSCRASQSVSSSYLAWYQQKPGQAPRLLIY
GASSRATGIPDRFSGSGSGTDFTLTISRLEPEDFAVYYCQQVGVVPYTFG
GGTKVEIK (SEQ ID NO: 570)
*Exemplary CDR sequences encompass amino acids as determined by Kabat & Chothia

TABLE 30
Antibody 43D7-CDR Sequences

| | | Exem- | | | | | |
|---|---|---|---|---|---|---|---|
| | | plary* | Kabat | Chothia | AbM | Contact | IMGT |
| VH | VH | GGSL | GYYW | GGSL | GGSL | SGYY | GGSL |
| CDR | CDR1 | SGYY | S | SGY | SGYY | WS | SGYY |
| Seq. | | WS | (SEQ | (SEQ | WS | (SEQ | (SEQ |
| | | (SEQ | ID | ID | (SEQ | ID | ID |
| | | ID | NO: | NO: | ID | NO: | NO: |
| | | NO: | 577) | 583) | NO: | 595) | 601) |
| | | 571) | | | 589) | | |
| | VH | EIGA | EIGA | ASG | EIGA | WIGE | IGAS |
| | CDR2 | SGST | SGST | (SEQ | SGST | IGAS | GST |
| | | RYNP | RYNP | ID | R | GSTR | (SEQ |
| | | SLKS | SLKS | NO: | (SEQ | (SEQ | ID |
| | | (SEQ | (SEQ | 584) | ID | ID | NO: |
| | | ID | ID | | NO: | NO: | 602) |
| | | NO: | NO: | | 590) | 596) | |
| | | 572) | 578) | | | | |
| | VH | DTPY | DTPY | TPYY | DTPY | ARDT | ARDT |
| | CDR3 | YYEG | YYEG | YEGG | YYEG | PYYY | PYYY |
| | | GYYY | GYYY | YYYY | GYYY | EGG | EGG |
| | | YMDV | YMDV | MD | YMDV | YYYY | YYYY |
| | | (SEQ | (SEQ | (SEQ | (SEQ | MD | MDV |
| | | ID | ID | ID | ID | (SEQ | (SEQ |
| | | NO: | NO: | NO: | NO: | ID | ID |
| | | 573) | 579) | 585) | 591) | NO: | NO: |
| | | | | | | 597) | 603) |
| VL | VL | RASD | RASD | SDSV | RASD | DSSY | DSVD |
| CDR | CDR1 | SVDS | SVDS | DSSY | SVDS | LAWY | SSY |
| Seq. | | SYLA | SYLA | (SEQ | SYLA | (SEQ | (SEQ |
| | | (SEQ | (SEQ | ID | (SEQ | ID | ID |
| | | ID | ID | NO: | ID | NO: | NO: |
| | | NO: | NO: | 586) | NO: | 598) | 604) |
| | | 574) | 580) | | 592) | | |
| | VL | GAFS | GAFS | GAF | GAFS | LLIY | GAF |
| | CDR2 | RAN | RAN | (SEQ | RAN | GAFS | (SEQ |
| | | (SEQ | (SEQ | ID | (SEQ | RA | ID |
| | | ID | ID | NO: | ID | (SEQ | NO: |
| | | NO: | NO: | 587) | NO: | ID | 605) |
| | | 575) | 581) | | 593) | NO: | |
| | | | | | | 599) | |
| | VL | QQAG | QQAG | AGVV | QQAG | QQAG | QQAG |
| | CDR3 | VVPY | VVPY | PY | VVPY | VVPY | VVPY |
| | | T | T | (SEQ | T | (SEQ | T |
| | | (SEQ | (SEQ | ID | (SEQ | ID | (SEQ |
| | | ID | ID | NO: | ID | NO: | ID |
| | | NO: | NO: | 588) | NO: | 600) | NO: |
| | | 576) | 582) | | 594) | | 606) |

VH Sequence*:
QVQLQQWGAGLLKPSETLSLTCAVYGGSLSGYYWSWIRQPPGKGLEWIGE
IGASGSTRYNPSLKSRVTISVDTSKNQFSLKLSSVTAADTAVYYCARDTP
YYYEGGYYYYMDVWGKGTTVTVSS (SEQ ID NO: 607)
VL Sequence*:
EIVLTQSPGTLSLSPGERATLSCRASDSVDSSYLAWYQQKPGQAPRLLIY
GAFSRANGIPDRFSGSGSGTDFTLTISRLEPEDFAVYYCQQAGVVPYTFG
GGTKVEIK (SEQ ID NO: 608)
*Exemplary CDR sequences encompass amino acids as determined by Kabat & Chothia

TABLE 31
Antibody 43D8-CDR Sequences

| | | Exem- | | | | | |
|---|---|---|---|---|---|---|---|
| | | plary* | Kabat | Chothia | AbM | Contact | IMGT |
| VH | VH | GGSL | GYYW | GGSL | GGSL | SGYY | GGSL |
| CDR | CDR1 | SGYY | S | SGY | SGYY | WS | SGYY |
| Seq. | | WS | (SEQ | (SEQ | WS | (SEQ | (SEQ |
| | | (SEQ | ID | ID | (SEQ | ID | ID |
| | | ID | NO: | NO: | ID | NO: | NO: |
| | | NO: | 615) | 621) | NO: | 633) | 639) |
| | | 609) | | | 627) | | |
| | VH | EIGA | EIGA | ASG | EIGA | WIGE | IGAS |
| | CDR2 | SGST | SGST | (SEQ | SGST | IGAS | GST |
| | | RYNP | RYNP | ID | R | GSTR | (SEQ |
| | | SLKS | SLKS | NO: | (SEQ | (SEQ | ID |
| | | (SEQ | (SEQ | 622) | ID | ID | NO: |
| | | ID | ID | | NO: | NO: | 640) |
| | | NO: | NO: | | 628) | 634) | |
| | | 610) | 616) | | | | |
| | VH | DTPY | DTPY | TPYY | DTPY | ARDT | ARDT |
| | CDR3 | YYEG | YYEG | YEGG | YYEG | PYYY | PYYY |
| | | GYYY | GYYY | YYYY | GYYY | EGG | EGG |
| | | YMDV | YMDV | MD | YMDV | YYYY | YYYY |
| | | (SEQ | (SEQ | (SEQ | (SEQ | MD | MDV |
| | | ID | ID | ID | ID | (SEQ | (SEQ |
| | | NO: | NO: | NO: | NO: | ID | ID |
| | | 611) | 617) | 623) | 629) | NO: | NO: |
| | | | | | | 635) | 641) |
| VL | VL | RASQ | RASQ | SQSV | RASQ | SSSF | QSVS |
| CDR | CDR1 | SVSS | SVSS | SSSF | SVSS | LAWY | SSF |
| Seq. | | SFLA | SFLA | (SEQ | SFLA | (SEQ | (SEQ |
| | | (SEQ | (SEQ | ID | (SEQ | ID | ID |
| | | ID | ID | NO: | ID | NO: | NO: |
| | | NO: | N0: | 624) | NO: | 636) | 642) |
| | | 612) | 618) | | 630) | | |
| | VL | GAYS | GAYS | GAY | GAYS | LLIY | GAY |
| | CDR2 | RAT | RAT | (SEQ | RAT | GAYS | (SEQ |
| | | (SEQ | (SEQ | ID | (SEQ | RA | ID |
| | | ID | ID | NO: | ID | (SEQ | NO: |
| | | NO: | NO: | 625) | NO: | ID | 643) |
| | | 613) | 619) | | 631) | NO: | |
| | | | | | | 637) | |
| | VL | QQAG | QQAG | AGVV | QQAG | QQAG | QQAG |
| | CDR3 | VVPY | VVPY | PY | VVPY | VVPY | VVPY |
| | | T | T | (SEQ | T | (SEQ | T |
| | | (SEQ | (SEQ | ID | (SEQ | ID | (SEQ |
| | | ID | ID | NO: | ID | NO: | ID |
| | | NO: | NO: | 626) | NO: | 638) | NO: |
| | | 614) | 620) | | 632) | | 644) |

VH Sequence*:
QVQLQQWGAGLLKPSETLSLTCAVYGGSLSGYYWSWIRQPPGKGLEWIGE
IGASGSTRYNPSLKSRVTISVDTSKNQFSLKLSSVTAADTAVYYCARDTP
YYYEGGYYYYMDVWGKGTTVTVSS (SEQ ID NO: 645)
VL Sequence*:
EIVLTQSPGTLSLSPGERATLSCRASQSVSSSFLAWYQQKPGQAPRLLIY
GAYSRATGIPDRFSGSGSGTDFTLTISRLEPEDFAVYYCQQAGVVPYTFG
GGTKVEIK (SEQ ID NO: 646)
*Exemplary CDR sequences encompass amino acids as determined by Kabat & Chothia

TABLE 32
Antibody 43E-CDR Sequences

| | | Exem- | | | | | |
|---|---|---|---|---|---|---|---|
| | | plary* | Kabat | Chothia | AbM | Contact | IMGT |
| VH | VH | GGSI | SGQY | GGSI | GGSI | SSGQ | GGSI |
| CDR | CDR1 | SSGQ | WS | SSGQ | SSGQ | YWS | SSGQ |
| Seq. | | YWS | (SEQ | (SEQ | YWS | (SEQ | Y |
| | | (SEQ | ID | ID | (SEQ | ID | (SEQ |
| | | ID | NO: | NO: | ID | NO: | ID |
| | | NO: | (653 | 659) | NO: | 671) | NO: |
| | | 647) | | | 665) | | 677) |
| | VH | EIYY | EIYY | YSG | EIYY | WIGE | IYYS |
| | CDR2 | SGST | SGST | (SEQ | SGST | IYYS | GST |
| | | RYNP | RYNP | ID | R | GSTR | (SEQ |
| | | SLKS | SLKS | NO: | (SEQ | (SEQ | ID |
| | | (SEQ | (SEQ | 660) | ID | ID | NO: |
| | | ID | ID | | NO: | NO: | 678) |
| | | NO: | NO: | | 666) | 672) | |
| | | 648) | 654) | | | | |
| | VH | DTPY | DTPY | TPYY | DTPY | ARDT | ARDT |
| | CDR3 | YYDG | YYDG | YDGG | YYDG | PYYY | PYYY |
| | | GYYY | GYYY | YYYY | GYYY | DGG | DGG |
| | | YMDV | YMDV | MD | YMDV | YYYY | YYYY |
| | | (SEQ | (SEQ | (SEQ | (SEQ | MD | MDV |
| | | ID | ID | ID | ID | (SEQ | (SEQ |
| | | NO: | NO: | NO: | NO: | ID | ID |
| | | 649) | 655) | 661) | 667) | NO: | NO: |
| | | | | | | 673) | 679) |
| VL | VL | RASQ | RASQ | SQSV | RASQ | SSSY | QSVS |
| CDR | CDR1 | SVSS | SVSS | SSSY | SVSS | LAWY | SSY |
| Seq. | | SYLA | SYLA | (SEQ | SYLA | (SEQ | (SEQ |
| | | (SEQ | (SEQ | ID | (SEQ | ID | ID |
| | | ID | ID | NO: | ID | NO: | NO: |
| | | NO: | NO: | 662) | NO: | 674) | 680) |
| | | 650) | 656) | | 668) | | |
| | VL | GASS | GASS | GAS | GASS | LLIY | GAS |
| | CDR2 | RAT | RAT | (SEQ | RAT | GASS | (SEQ |
| | | (SEQ | (SEQ | ID | (SEQ | RA | ID |
| | | ID | ID | NO: | ID | (SEQ | NO: |
| | | NO: | NO: | 663) | NO: | ID | 681) |
| | | 651) | 657) | | 669) | NO: | |
| | | | | | | 675) | |
| | VL | QQVG | QQVG | VGVV | QQVG | QQVG | QQVG |
| | CDR3 | VVPY | VVPY | PY | VVPY | VVPY | VVPY |
| | | T | T | (SEQ | T | (SEQ | T |
| | | (SEQ | (SEQ | ID | (SEQ | ID | (SEQ |
| | | ID | ID | NO: | ID | NO: | ID |
| | | NO: | NO: | 664) | NQ: | 676) | NO: |
| | | 652) | 658) | | 670) | | 682) |

VH Sequence*:
QVQLQESGPGLVKPSQTLSLTCTVSGGSISSGQYWSWIRQHPGKGLEWIG
EIYYSGSTRYNPSLKSRVTISVDTSKDQFSLKLSSVTAADTAVYYCARDT
PYYYDGGYYYYMDVWGKGTTVTVSS (SEQ ID NO: 683)
VL Sequence*:
EIVLTQSPGTLSLSPGERATLSCRASQSVSSSYLAWYQQKPGQAPRLLIY
GASSRATGIPDRFSGSGSGTDFTLTISRLEPEDFAVYYCQQVGVVPYTFG
GGTKVEIK (SEQ ID NO: 684)
*Exemplary CDR sequences encompass amino acids as determined by Kabat & Chothia

TABLE 33
Antibody 43Ea-CDR Sequences

| | | Exem- | | | | | |
|---|---|---|---|---|---|---|---|
| | | plary* | Kabat | Chothia | AbM | Contact | IMGT |
| VH | VH | GGSI | SGQY | GGSI | GGSI | SSGQ | GGSI |
| CDR | CDR1 | SSGQ | WS | SSGQ | SSGQ | YWS | SSGQ |
| Seq. | | YWS | (SEQ | (SEQ | YWS | (SEQ | Y |
| | | (SEQ | ID | ID | (SEQ | ID | (SEQ |
| | | ID | NO: | NO: | ID | NO: | ID |
| | | NO: | 691) | 697) | NO: | 709) | NO: |
| | | 685) | | | 703) | | 715) |
| | VH | EIYY | EIYY | YSG | EIYY | WIGE | IYYS |
| | CDR2 | SGST | SGST | (SEQ | SGST | IYYS | GST |
| | | RYNP | RYNP | ID | R | GSTR | (SEQ |
| | | SLKS | SLKS | NO: | (SEQ | (SEQ | ID |
| | | (SEQ | (SEQ | 698) | ID | ID | NO: |
| | | ID | ID | | NO: | NO: | 716) |
| | | NO: | NO: | | 704) | 710) | |
| | | 686) | 692) | | | | |
| | VH | DTPY | DTPY | TPYY | DTPY | ARDT | ARDT |
| | CDR3 | YYDG | YYDG | YDGG | YYDG | PYYY | PYYY |
| | | GYYY | GYYY | YYYY | GYYY | DGG | DGG |
| | | YMDV | YMDV | MD | YMDV | YYYY | YYYY |
| | | (SEQ | (SEQ | (SEQ | (SEQ | MD | MDV |
| | | ID | ID | ID | ID | (SEQ | (SEQ |
| | | NO: | NO: | NO: | NO: | ID | ID |
| | | 687) | 693) | 699) | 705) | NO: | NO: |
| | | | | | | 711) | 717) |
| VL | VL | RASQ | RASQ | SQSV | RASQ | SSSY | QSVS |
| CDR | CDR1 | SVSS | SVSS | SSSY | SVSS | LAWY | SSY |
| Seq. | | SYLA | SYLA | (SEQ | SYLA | (SEQ | (SEQ |
| | | (SEQ | (SEQ | ID | (SEQ | ID | ID |
| | | ID | ID | NO: | ID | NO: | NO: |
| | | NO: | NO: | 700) | NO: | 712) | 718) |
| | | 688) | 694) | | 706) | | |
| | VL | GASS | GASS | GAS | GASS | LLIY | GAS |
| | CDR2 | RAT | RAT | (SEQ | RAT | GASS | (SEQ |
| | | (SEQ | (SEQ | ID | (SEQ | RA | ID |
| | | ID | ID | NO: | ID | (SEQ | NO: |
| | | NO: | NO: | 701) | NO: | ID | 719) |
| | | 689) | 695) | | 707) | NO: | |
| | | | | | | 713) | |
| | VL | QQVG | QQVG | VGVV | QQVG | QQVG | QQVG |
| | CDR3 | VVPY | VVPY | PY | VVPY | VVPY | VVPY |
| | | T | T | (SEQ | T | (SEQ | T |
| | | (SEQ | (SEQ | ID | (SEQ | ID | (SEQ |
| | | ID NO: | ID | NO: | ID | NO: | ID |
| | | 690) | NO: | 702) | NO: | 714) | NO: |
| | | | 696) | | 708) | | 720) |

VH Sequence*:
QVQLQESGPGLVKPSQTLSLTCTVSGGSISSGQYWSWIRQHPGKGLEWIG
EIYYSGSTRYNPSLKSRVTISVDTSNQFSLKLSSVTAADTAVYYCARDTP
YYYDGGYYYYMDVWGKGTTVTVSS (SEQ ID NO: 721)
VL Sequence*:
EIVLTQSPGTLSLSPGERATLSCRASQSVSSSYLAWYQQKPGQAPRLLIY
GASSRATGIPDRFSGSGSGTDFTLTISRLEPEDFAVYYCQQVGVVPYTFG
GGTKVEIK (SEQ ID NO: 722)
*Exemplary CDR sequences encompass amino acids as determined by Kabat & Chothia

TABLE 34
Antibody 54E-CDR Sequences

| | | Exem- | | | | | |
|---|---|---|---|---|---|---|---|
| | | plary* | Kabat | Chothia | AbM | Contact | IMGT |
| VH | VH | GYTF | NYYM | GYTF | GYTF | ANYY | GYTF |
| CDR | CDR1 | ANYY | H | ANY | ANYY | MH | ANYY |
| Seq. | | MH | (SEQ | (SEQ | MH | (SEQ | (SEQ |
| | | (SEQ | ID | ID | (SEQ | ID | ID |
| | | ID | NO: | NO: | ID | NO: | NO: |
| | | NO: | 729) | 735) | NO: | 747) | 753) |
| | | 723) | | | 741) | | |
| | VH | IINP | IINP | PSGG | IINP | WMGI | INPS |
| | CDR2 | SGGI | SGGI | (SEQ | SGGI | INPS | GGIT |
| | | TVYA | TVYA | ID | TV | GGIT | (SEQ |
| | | Q | Q | NO: | (SEQ | V | ID |
| | | KFQG | KFQG | 736) | ID | (SEQ | NO: |
| | | (SEQ | (SEQ | | NO: | ID | 754) |
| | | ID | ID | | 742) | NO: | |
| | | NO: | NO: | | | 748) | |
| | | 724) | 730) | | | | |
| | VH | GGSK | GGSK | GSKV | GGSK | ARGG | ARGG |
| | CDR3 | VAAL | VAAL | AALA | VAAL | SKVA | SKVA |
| | | AFDI | AFDI | FD | AFDI | ALA | ALA |
| | | (SEQ | (SEQ | (SEQ | (SEQ | FD | FDI |
| | | ID | ID | ID | ID | (SEQ | (SEQ |
| | | NO: | NO: | NO: | NO: | ID | ID |
| | | 725) | 731) | 737) | 743) | NO: | NO: |
| | | | | | | 749) | 755) |
| VL | VL | QASQ | QASQ | SQDI | QASQ | SNSL | QDIS |
| CDR | CDR1 | DISN | DISN | SNS | DISN | NWY | NS |
| Seq. | | SLN | SLN | (SEQ | SLN | (SEQ | (SEQ |
| | | (SEQ | (SEQ | ID | (SEQ | ID | ID |
| | | ID | ID | NO: | ID | NO: | NO: |
| | | NO: | NO: | 738) | NO: | 750) | 756) |
| | | 726) | 732) | | 744) | | |
| | VL | DASN | DASN | DAS | DASN | LLIY | DAS |
| | CDR2 | LET | LET | (SEQ | LET | DASN | (SEQ |
| | | (SEQ | (SEQ | ID | (SEQ | LE | ID |
| | | ID | ID | NO: | ID | (SEQ | NO: |
| | | NO: | NO: | 739) | NO: | ID | 757) |
| | | 727) | 733) | | 745) | NO: | |
| | | | | | | 751) | |
| | VL | QQYN | QQYN | YNFH | QQYN | QQYN | QQYN |
| | CDR3 | FHPL | FHPL | PL | FHPL | FHPL | FHPL |
| | | T | T | (SEQ | T | (SEQ | T |
| | | (SEQ | (SEQ | ID | (SEQ | ID | (SEQ |
| | | ID | ID | NQ: | ID | NO: | ID |
| | | NO: | NO: | 740) | NO: | 752) | NO: |
| | | 728) | 734) | | 746) | | 758) |

VH Sequence*:
QVQLVQSGAEVKKPGASVKVSCKASGYTFANYYMHWVRQAPGQGLEWMGI
INPSGGITVYAQKFQGRVTMTRTDSTSTVYMELSSLRSEDTAVYYCARGG
SKVAALAFDIWGQGTMVTVSS (SEQ ID NO: 759)
VL Sequence*:
DIQMTQSPSSLSASVGDRVTITCQASQDISNSLNWYQQKPGKAPKLLIY
DASNLETGVPSRFSGSRSGTDFTFTISSLQPEDIATYYCQQYNFHPLTF
GGGTKVEIK (SEQ ID NO: 760)
*Exemplary CDR sequences encompass amino acids as determined by Kabat plus Chothia

TABLE 35
Consensus CDRs

| | | Antibody Group | | | | | |
|---|---|---|---|---|---|---|---|
| | | 1 | 25 | 29 | 39 | 43 | 54 |
| VH | VH | GFTFS | GYTF | GFTF | GGTF | GGSx | GYTF |
| CDR | CDR1 | [x[D/ | x[D/R] | x[H/R] | SSNA | [I/L] | ANYY |
| Seq.* | | S] | x[S/V] | Sx[R/ | IG | SSGx | MH |
| | | YAMx | YGIS | Y]GMH | (SEQ | [Q/Y] | (SEQ |
| | | [A/G] | (SEQ | (SEQ | ID | YWS | ID |
| | | (SEQ | ID | ID | NO: | (SEQ | NO: |
| | | ID | NO: | NO: | 791) | ID | 803) |
| | | NO: | 779) | 785) | | NO: | |
| | | 773) | | | | 797) | |
| | VH | x[A/T] | Wx[I/V] | VITY | SIIP | EIx | IINP |
| | CDR2 | ISGS | APYx | DGIN | IIGF | [Y/G] | SGGI |
| | | GGLT | [S/N] | KYYA | ANYA | x[Y/A] | TVYA |
| | | YYAD | GNTNY | DSVE | QK | SGST | QKFQ |
| | | SVKG | AQKL | G | FQG | RYNP | G |
| | | (SEQ | QG | (SEQ | (SEQ | SLKS | (SEQ |
| | | ID | (SEQ | ID | ID | (SEQ | ID |
| | | NO: | ID | NO: | NO: | ID | NO: |
| | | 774) | NO: | 786) | 792) | NO: | 804) |
| | | | 780) | | | 798) | |
| | VH | APYG | DAGT | DGVY | DSGY | Dx[T/A] | GGSK |
| | CDR3 | YYMD | YSPx | YGVY | YYGA | PYYYx | VAAL |
| | | V | [F/Y] | DY | SSFG | [E/G/D] | AFDI |
| | | (SEQ | GYG | (SEQ | MDV | GGYYY | (SEQ |
| | | ID | MDV | ID | (SEQ | YMDV | ID |
| | | NO: | (SEQ | NO: | ID | (SEQ | NO: |
| | | 775) | ID | 787) | NO: | ID | 805) |
| | | | NO: | | 793) | NO: | |
| | | | 781) | | | 799) | |
| VL | VL | RASQ | x[R/Q] | KSSQ | RASQ | RASx | QASQ |
| CDR | CDR1 | SISS | Asx | SVLF | SV | [Q/ | DI |
| Seq.* | | WLA | [Q/E/ | SS | SS | E/D] | SN |
| | | (SEQ | H] | NN | NLA | SVx | SLN |
| | | ID | SIx[S/ | KNY | (SEQ | [S/D] | (SEQ |
| | | NO: | D/N] | LA | ID | SSx | ID |
| | | 776) | x[S/N] | (SEQ | NO: | [Y/F] | NO: |
| | | | WLA | ID | 794) | LA | 806) |
| | | | (SEQ | NO: | | (SEQ | |
| | | | ID | 788) | | ID | |
| | | | NO: | | | NO: | |
| | | | 782) | | | 800) | |
| | VL | KASS | x[K/S] | WAST | GAST | GAx | DASN |
| | CDR2 | LES | Ax | RES | RAT | [S/D/ | LET |
| | | (SEQ | [S/Y] | (SEQ | (SEQ | F/Y] | (SEQ |
| | | ID | x[S/ | ID | ID | x[S/T] | ID |
| | | NO: | Y/N] | NO: | NO: | Rx | NO: |
| | | 777) | LEx | 789) | 795) | [A/Q] | 807) |
| | | | [S/Y] | | | x[T/N] | |
| | | | (SEQ | | | (SEQ | |
| | | | ID | | | ID | |
| | | | NO: | | | NO: | |
| | | | 783) | | | 801) | |
| | VL | QQYK | Qx[Q/ | QQFH | EQYN | QQx | QQYN |
| | CDR3 | SYIT | L/R] | SYPL | NLPL | [V/A/ | FHPL |
| | | (SEQ | FQx | T | T | D]GV | T |
| | | ID | [S/K] | (SEQ | (SEQ | VPYT | (SEQ |
| | | NO: | LPPF | ID | ID | (SEQ | ID |
| | | 778) | T | NO: | NO: | ID | NO: |
| | | | (SEQ | 790) | 796) | NO: | 808) |
| | | | ID | | | 802) | |
| | | | NO: | | | | |
| | | | 784) | | | | |

*Exemplary CDR sequences encompass amino acids as determined by Kabat plus Chothia

TABLE 36
Human, Cynomolgus Monkey, and Mouse TF Sequences

| | Species | | |
|---|---|---|---|
| | | Cynomolgus | |
| | Human | Monkey | Mouse |
| | (Homo | (Macaca | (Mus |
| | sapiens) | fascicularis) | musculus) |
| Full-length | METPAWPRVP | METPAWPRVP | MAILVRPRLL |
| sequence | RPETAVARTL | RPETAVARTL | AALAPTFLGC |
| [signal | LLGWVFAQVA | LLGWVFAQVA | LLLQVTAGAG |
| sequence | GASGTTNTVA | GASGTTNTVA | IPEKAFNLTW |
| underlined] | AYNLTWKSTN | AYNLTWKSTN | ISTDFKTILE |
| | FKTILEWEPK | FKTILEWEPK | WQPKPTNYTY |
| | PVNQVYTVQI | PINQVYTVQI | TVQISDRSRN |
| | STKSGDWKSK | STKSGDWKSK | WKNKCFSTTD |
| | CFYTTDTECD | CFYTADTECD | TECDLTDEIV |
| | LTDEIVKDVK | LTDEIVKDVK | KDVTWAYEAK |
| | QTYLARVFSY | QTYLARVFSY | VLSVPRRNSV |
| | PAGNVESTGS | PAGHVESTGS | HGDGDQLVIH |
| | AGEPLYENSP | TEEPPYENSP | GEEPPFTNAP |
| | EFTPYLETNL | EFTPYLETNL | KFLPYRDTNL |
| | GQPTIQSFEQ | GQPTIQSFEQ | GQPVIQQFEQ |
| | VGTKVNVTVE | VGTKVNVTVQ | DGRKLNVVVK |
| | DERTLVRRNN | DEWTLVRRND | DSLTLVRKNG |
| | TFLSLRDVFG | TFLSLRDVFG | TFLTLRQVFG |
| | KDLIYTLYYW | KDLIYTLYYW | KDLGYIITYR |
| | KSSSSGKKTA | KSSSSGKKTA | KGSSTGKKTN |
| | KTNTNEFLID | KTNTNEFLID | ITNTNEFSID |
| | VDKGENYCFS | VDKGENYCFS | VEEGVSYCFF |
| | VQAVIPSRTV | VQAVIPSRRT | VQAMIFSRKT |
| | NRKSTDSPVE | ANRKSTDSPV | NQNSPGSSTV |
| | CMGQEKGEFR | ECMGHEKGES | CTEQWKSFLG |
| | EIFYIIGAVV | REIFYIIGAV | ETLIIVGAVV |
| | FVVIILVIIL | VFVVIILVII | LLATIFIILL |
| | AISLHKCRKA | LAISLHKCK | SISLCKRRKN |
| | GVGQSWKENS | KARVGRS | RAGQKGKNTP |
| | PLNVS | WKENSPLNVA | SRLA |
| | (SEQ ID | (SEQ ID | (SEQ ID |
| | NO: 809) | NO: 813) | NO: 817) |
| Extra- | SGTTNTVAAY | SGTTNTVAAY | AGIPEKAFNL |
| cellular | NLTWKSTNFK | NLTWKSTNFK | TWISTDFKTI |
| domain | TILEWEPKPV | TILEWEPKPI | LEWQPKPTNY |
| (ECD) | NQVYTVQIST | NQVYTVQIST | TYTVQISDRS |
| | KSGDWKSKCF | KSGDWKSKCF | RNWKNKCFST |
| | YTTDTECDLT | YTADTECDLT | TDTECDLTDE |
| | DEIVKDVKQT | DEIVKDVKQT | IVKDVTWAYE |
| | YLARVFSYPA | YLARVFSYPA | AKVLSVPRRN |
| | GNVESTGSAG | GHVESTGSTE | SVHGDGDQLV |
| | EPLYENSPEF | EPPYENSPEF | IHGEEPPFTN |
| | TPYLETNLGQ | TPYLETNLGQ | APKFLPYRDT |
| | PTIQSFEQVG | PTIQSFEQVG | NLGQPVIQQF |
| | TKVNVTVEDE | TKVNVTVQDE | EQDGRKLNVV |
| | RTLVRRNNTF | WTLVRRNDTF | VKDSLTLVRK |
| | LSLRDVFGKD | LSLRDVFGKD | NGTFLTLRQV |
| | LIYTLYYWKS | LIYTLYYWKS | FGKDLGYIIT |
| | SSSGKKTAKT | SSSGKKTAKT | YRKGSSTGKK |
| | NTNEFLIDVD | NTNEFLIDVD | TNITNTNEFS |
| | KGENYCFSVQ | KGENYCFSVQ | IDVEEGVSYC |
| | AVIPSRTVNR | AVIPSRRTAN | FFVQAMIFSR |
| | KSTDSPVECM | RKSTDSPVEC | KTNQNSPGSS |
| | GQEKGEFRE | MGHEKGESRE | TVCTEQWKSF |
| | (SEQ ID | (SEQ ID | LGE |
| | NO: 810) | NO: 814) | (SEQ ID |
| | | | NO: 818) |
| Sequence of | SGTTNTVAAY | SGTTNTVAAY | AGIPEKAFNL |
| TF ECD-His | NLTWKSTNFK | NLTWKSTNFK | TWISTDFKTI |
| (TF-His) | TILEWEPKPV | TILEWEPKPI | LEWQPKPTNY |
| protein | NQVYTVQIST | NQVYTVQIST | TYTVQISDRS |
| | KSGDWKSKCF | KSGDWKSKCF | RNWKNKCFST |
| | YTTDTECDLT | YTADTECDLT | TDTECDLTDE |
| | DEIVKDVKQT | DEIVKDVKQT | IVKDVTWAYE |
| | YLARVFSYPA | YLARVFSYPA | AKVLSVPRRN |
| | GNVESTGSAG | GHVESTGSTE | SVHGDGDQLV |
| | EPLYENSPEF | EPPYENSPEF | IHGEEPPFTN |
| | TPYLETNLGQ | TPYLETNLGQ | APKFLPYRDT |
| | PTIQSFEQVG | PTIQSFEQVG | NLGQPVIQQF |
| | TKVNVTVEDE | TKVNVTVQDE | EQDGRKLNVV |
| | RTLVRRNNTF | WTLVRRNDTF | VKDSLTLVRK |
| | LSLRDVFGKD | LSLRDVFGKD | NGTFLTLRQV |
| | LIYTLYYWKS | LIYTLYYWKS | FGKDLGYIIT |
| | SSSGKKTAKT | SSSGKKTAKT | YRKGSSTGKK |
| | NTNEFLIDVD | NTNEFLIDVD | TNITNTNEFS |
| | KGENYCFSVQ | KGENYCFSVQ | IDVEEGVSYC |
| | AVIPSRTVNR | AVIPSRRTAN | FFVQAMIFSR |
| | KSTDSPVECM | RKSTDSPVEC | KTNQNSPGSS |
| | GQEKGEFRET | MGHEKGESRE | TVCTEQWKSF |
| | GHHHHHH | TGHHHHHH | LGETGHHHHH |
| | (SEQ ID | (SEQ ID | H |
| | NO: 811) | NO: 815) | (SEQ ID |
| | | | NO: 819) |
| Sequence of | SGTTNTVAAY | SGTTNTVAAY | AGIPEKAFNL |
| TF ECD-Fc | NLTWKSTNFK | NLTWKSTNFK | TWISTDFKTI |
| (TF-Fc) | TILEWEPKPV | TILEWEPKPI | LEWQPKPTNY |
| fusion | NQVYTVQIST | NQVYTVQIST | TYTVQISDRS |
| protein | KSGDWKSKCF | KSGDWKSKCF | RNWKNKCFST |
| | YTTDTECDLT | YTADTECDLT | TDTECDLTDE |
| | DEIVKDVKQT | DEIVKDVKQT | IVKDVTWAYE |
| | YLARVFSYPA | YLARVFSYPA | AKVLSVPRRN |
| | GNVESTGSAG | GHVESTGSTE | SVHGDGDQLV |
| | EPLYENSPEF | EPPYENSPEF | IHGEEPPFTN |
| | TPYLETNLGQ | TPYLETNLGQ | APKFLPYRDT |
| | PTIQSFEQVG | PTIQSFEQVG | NLGQPVIQQF |
| | TKVNVTVEDE | TKVNVTVQDE | EQDGRKLNVV |
| | RTLVRRNNTF | WTLVRRNDTF | VKDSLTLVRK |
| | LSLRDVFGKD | LSLRDVFGKD | NGTFLTLRQV |
| | LIYTLYYWKS | LIYTLYYWKS | FGKDLGYIIT |
| | SSSGKKTAKT | SSSGKKTAKT | YRKGSSTGKK |
| | NTNEFLIDVD | NTNEFLIDVD | TNITNTNEFS |
| | KGENYCFSVQ | KGENYCFSVQ | IDVEEGVSYC |
| | AVIPSRTVNR | AVIPSRRTAN | FFVQAMIFSR |
| | KSTDSPVECM | RKSTDSPVEC | KTNQNSPGSS |
| | GQEKGEFRET | MGHEKGESRE | TVCTEQWKSF |
| | GENLYFQGDK | TGENLYFQGD | LGETGENLYF |
| | THTCPPCPAP | KTHTCPPCPA | QGDKTHTCPP |
| | ELLGGPSVFL | PELLGGPSVF | CPAPELLGGP |
| | FPPKPKDTLM | LFPPKPKDTL | SVFLFPPKPK |
| | ISRTPEVTCV | MISRTPEVTC | DTLMISRTPE |
| | VVDVSHEDPE | VVVDVSHEDP | VTCVVVDVSH |
| | VKFNWYVDGV | EVKFNWYVDG | EDPEVKFNWY |
| | EVHNAKTKPR | VEVHNAKTKP | VDGVEVHNAK |
| | EEQYNSTYRV | REEQYNSTYR | TKPREEQYNS |
| | VSVLTVLHQD | VVSVLTVLHQ | TYRVVSVLTV |
| | WLNGKEYKCK | DWLNGKEYKC | LHQDWLNGKE |
| | VSNKALPAPI | KVSNKALPAP | YKCKVSNKAL |
| | EKTISKAKGQ | IEKTISKAKG | PAPIEKTISK |
| | PREPQVYTLP | QPREPQVYTL | AKGQPREPQV |
| | PSREEMTKNQ | PPSREEMTKN | YTLPPSREEM |
| | VSLTCLVKGF | QVSLTCLVKG | TKNQVSLTCL |
| | YPSDIAVEWE | FYPSDIAVEW | VKGFYPSDIA |
| | SNGQPENNYK | ESNGQPENNY | VEWESNGQPE |
| | TTPPVLDSDG | KTTPPVLDSD | NNYKTTPPVL |
| | SFFLYSKLTV | GSFFLYSKLT | DSDGSFFLYS |
| | DKSRWQQGNV | VDKSRWQQGN | KLTVDKSRWQ |
| | FSCSVMHEAL | VFSCSVMHEA | QGNVFSCSVM |
| | HNHYTQKSLS | LHNHYTQKSL | HEALHNHYTQ |
| | LSPGK | SLSPGK | KSLSLSPGK |
| | (SEQ ID | (SEQ ID | (SEQ ID |
| | NO: 812) | NO: 816) | NO: 820) |

TABLE 39
Sequences of Anti-TF Antibodies

| Antibody | VH domain | VL domain |
|---|---|---|
| 10H10 | EVQLVQSGAEVKKPG | DIVMTQTPLSLPVTP |
| (M1593) | ESLRISCKGSGYTFA | GEPASISCKSSQSLL |
| | PYWIEWVRQMPGKGL | SSGNQKNYLTWYLQK |
| | EWMGDILPGTGFTTY | PGQSPQLLIYWASTR |
| | SPSFQGHVTISADKS | ESGVPDRFSGSGSGT |
| | ISTAYLQWSSLKASD | DFTLKISRVEAEDVG |
| | TAMYYCARSGYYGNS | VYYCQNDYTYPLTFG |
| | GFAYWGQGTLVTVSS | QGTKLEIK |
| | (SEQ ID NO: 821) | (SEQ ID NO: 822) |
| TF-011 | EVQLLESGGGLVQPG | DIQMTQSPPSLSASA |
| | GSLRLSCAASGFTFS | GDRVTITCRASQGIS |
| | NYAMSWVRQAPGKGL | SRLAWYQQKPEKAPK |
| | EWVSSISGSGDYTYY | SLIYAASSLQSGVPS |
| | TDSVKGRFTISRDNS | RFSGSGSGTDFTLTI |
| | KNTLYLQMNSLRAED | SSLQPEDFATYYCQQ |
| | TAVYYCARSPWGYYL | YNSYPYTFGQGTKLE |
| | DSWGQGTLVTVSS | IK |
| | (SEQ ID NO: 828) | (SEQ ID NO: 829) |
| 5G9 | QVQLVESGGGVVQPG | DIQMTQSPSSLSASV |
| (humanized | RSLRLSCKASGFNIK | GDRVTITCKASQDIR |
| TF8-5G9, | DYYMHWVRQAPGKGL | KYLNWYQQKPGKAPK |
| CNTO 860) | EWIGLIDPENGNTIY | LLIYYATSLADGVPS |
| | DPKFQGRFTISADNS | RFSGSGSGTDYTFTI |
| | KNTLFLQMDSLRPED | SSLQPEDIATYYCLQ |
| | TAVYYCARDNSYYFD | HGESPYTFGQGTKLE |
| | YWGQGTPVTVSS | IT |
| | (SEQ ID NO: 830) | (SEQ ID NO: 831) |

TABLE 41
Pig TF sequences

| | Species |
|---|---|
| | Pig (Sus scrofa) |
| Full-length | MATPTGPPVSCPKAAVARALLLGWV |
| sequence | LVQVAGATGTTDVIVAYNLTWKSTN |
| [signal | FKTILEWEPKPINYVYTVQISPRLG |
| sequence | DWKNKCFHTTDTECDVTDEIMRNVK |
| underlined] | ETYVARVLSYPADTVLTAQEPPFTN |
| | SPPFTPYLDTNLGQPVIQSFEQVGT |
| | KLNVTVEAARTLVRVNGTFLRLRDV |
| | FGKDLNYTLYYWRASSTGKKKATTN |
| | TNEFLIDVDKGENYCFSVQAVIPSR |
| | RVNQKSPESRIECTSQEKAVSRELF |
| | LIVGAVVFAVIVFVLVLSVSLYKCR |
| | KERAGPSGKENAPLNVA |
| | (SEQ ID NO: 824) |
| Extracellular | TGTTDVIVAYNLTWKSTNFKTILEW |
| domain | EPKPINYVYTVQISPRLGDWKNKCF |
| (ECD) | HTTDTECDVTDEIMRNVKETYVARV |
| | LSYPADTVLTAQEPPFTNSPPFTPY |
| | LDTNLGQPVIQSFEQVGTKLNVTVE |
| | AARTLVRVNGTFLRLRDVFGKDLNY |
| | TLYYWRASSTGKKKATTNTNEFLID |
| | VDKGENYCFSVQAVIPSRRVNQKSP |
| | ESRIECTSQEKAVSRE |
| | (SEQ ID NO: 825) |
| Sequence of | TGTTDVIVAYNLTWKSTNFKTILEW |
| TF ECD-His | EPKPINYVYTVQISPRLGDWKNKCF |
| (TF-His) | HTTDTECDVTDEIMRNVKETYVARV |
| protein | LSYPADTVLTAQEPPFTNSPPFTPY |
| | LDTNLGQPVIQSFEQVGTKLNVTVE |
| | AARTLVRVNGTFLRLRDVFGKDLNY |
| | TLYYWRASSTGKKKATTNTNEFLID |
| | VDKGENYCFSVQAVIPSRRVNQKSP |
| | ESRIECTSQEKAVSRETGHHHHHH |
| | (SEQ ID NO: 826) |
| Sequence of | TGTTDVIVAYNLTWKSTNFKTILEW |
| TF ECD-Fc | EPKPINYVYTVQISPRLGDWKNKCF |
| (TF-Fc) | HTTDTECDVTDEIMRNVKETYVARV |
| fusion | LSYPADTVLTAQEPPFTNSPPFTPY |
| protein | LDTNLGQPVIQSFEQVGTKLNVTVE |
| | AARTLVRVNGTFLRLRDVFGKDLNY |
| | TLYYWRASSTGKKKATTNTNEFLID |
| | VDKGENYCFSVQAVIPSRRVNQKSP |
| | ESRIECTSQEKAVSRETGENLYFQG |
| | DKTHTCPPCPAPELLGGPSVFLFPP |
| | KPKDTLMISRTPEVTCVVVDVSHED |
| | PEVKFNWYVDGVEVHNAKTKPREEQ |
| | YNSTYRVVSVLTVLHQDWLNGKEYK |
| | CKVSNKALPAPIEKTISKAKGQPRE |
| | PQVYTLPPSREEMTKNQVSLTCLVK |
| | GFYPSDIAVEWESNGQPENNYKTTP |
| | PVLDSDGSFFLYSKLTVDKSRWQQG |
| | NVFSCSVMHEALHNHYTQKSLSLSP |
| | GK (SEQ ID NO: 827) |

TABLE 49
Rabbit TF sequences

| | Species |
|---|---|
| | Rabbit |
| | (Oryctolagus cuniculus) |
| Full-length | MAPPTRLQVPRPGTAVPYTV |
| sequence | LLGWLLAQVARAADTTGRAY |
| [signal | NLTWKSTNFKTILEWEPKSI |
| sequence | DHVYTVQISTRLENWKSKCF |
| underlined] | LTAETECDLTDEVVKDVGQT |
| | YMARVLSYPARNGNTTGFPE |
| | EPPFRNSPEFTPYLDTNLGQ |
| | PTIQSFEQVGTKLNVTVQDA |
| | RTLVRRNGTFLSLRAVFGKD |
| | LNYTLYYWRASSTGKKTATT |
| | NTNEFLIDVDKGENYCFSVQ |
| | AVIPSRKRKQRSPESLTECT |
| | SREQGRAREMFFIIGAVVVV |
| | ALLIIVLSVTVYKCRKARAG |
| | PSGKESSPLNIA |
| | (SEQ ID NO: 832) |
| Extracellular | ADTTGRAYNLTWKSTNFKTI |
| domain (ECD) | LEWEPKSIDHVYTVQISTRL |
| | ENWKSKCFLTAETECDLTDE |
| | VVKDVGQTYMARVLSYPARN |
| | GNTTGFPEEPPFRNSPEFTP |
| | YLDTNLGQPTIQSFEQVGTK |
| | LNVTVQDARTLVRRNGTFLS |
| | LRAVFGKDLNYTLYYWRASS |
| | TGKKTATTNTNEFLIDVDKG |
| | ENYCFSVQAVIPSRKRKQRS |
| | PESLTECTSREQGRAREM |
| | (SEQ ID NO: 833) |
| Sequence of | ADTTGRAYNLTWKSTNFKTI |
| TF ECD-His | LEWEPKSIDHVYTVQISTRL |
| (TF-His) | ENWKSKCFLTAETECDLTDE |
| protein | VVKDVGQTYMARVLSYPARN |
| | GNTTGFPEEPPFRNSPEFTP |
| | YLDTNLGQPTIQSFEQVGTK |
| | LNVTVQDARTLVRRNGTFLS |
| | LRAVFGKDLNYTLYYWRASS |
| | TGKKTATTNTNEFLIDVDKG |
| | ENYCFSVQAVIPSRKRKQRS |
| | PESLTECTSREQGRAREMTG |
| | HHHHHH |
| | (SEQ ID NO: 834) |
| Sequence of | ADTTGRAYNLTWKSTNFKTI |
| TF ECD-Fc | LEWEPKSIDHVYTVQISTRL |
| (TF-Fc) | ENWKSKCFLTAETECDLTDE |
| fusion | VVKDVGQTYMARVLSYPARN |
| protein | GNTTGFPEEPPFRNSPEFTP |
| | YLDTNLGQPTIQSFEQVGTK |
| | LNVTVQDARTLVRRNGTFLS |
| | LRAVFGKDLNYTLYYWRASS |
| | TGKKTATTNTNEFLIDVDKG |
| | ENYCFSVQAVIPSRKRKQRS |
| | PESLTECTSREQGRAREMEN |
| | LYFQGDKTHTCPPCPAPELL |
| | GGPSVFLFPPKPKDTLMISR |
| | TPEVTCVVVDVSHEDPEVKF |
| | NWYVDGVEVHNAKTKPREEQ |
| | YNSTYRVVSVLTVLHQDWLN |
| | GKEYKCKVSNKALPAPIEKT |
| | ISKAKGQPREPQVYTLPPSR |
| | EEMTKNQVSLTCLVKGFYPS |
| | DIAVEWESNGQPENNYKTTP |
| | PVLDSDGSFFLYSKLTVDKS |
| | RWQQGNVFSCSVMHEALHNH |
| | YTQKSLSLSPGK |
| | (SEQ ID NO: 835) |

TABLE 56
Rat TF ECD and chimeric
construct ECD sequences

| Rat/Chimeric | Extracellular domain |
|---|---|
| construct | (ECD) sequence |
| rTF (rat TF) | AGTPPGKAFNLTWISTDFKTILEWQ |
| | PKPTNYTYTVQISDRSRNWKYKCTG |
| | TTDTECDLTDEIVKDVNWTYEARVL |
| | SVPWRNSTHGKETLFGTHGEEPPFT |
| | NARKFLPYRDTKIGQPVIQKYEQGG |
| | TKLKVTVKDSFTLVRKNGTFLTLRQ |
| | VFGNDLGYILTYRKDSSTGRKTNTT |
| | HTNEFLIDVEKGVSYCFFAQAVIFS |
| | RKTNHKSPESITKCTEQWKSVLGE |
| | (SEQ ID NO: 838) |
| h1-107_r | AGTPPGKAFNLTWISTDFKTILEWQ |
| | PKPTNYTYTVQISDRSRNWKYKCTG |
| | TTDTECDLTDEIVKDVNWTYEARVL |
| | SVPWRNSTHGKETLFGTHGEEPPFT |
| | NARKFLPYRDTKLGQPTIQSFEQVG |
| | TKVNVTVEDERTLVRRNNTFLSLRD |
| | VFGKDLIYTLYYWKSSSSGKKTAKT |
| | NTNEFLIDVDKGENYCFSVQAVIPS |
| | RTVNRKSTDSPVECMGQEKGEFRE |
| | (SEQ ID NO: 839) |
| h1-77_r | AGTPPGKAFNLTWISTDFKTILEWQ |
| | PKPTNYTYTVQISDRSRNWKYKCTG |
| | TTDTECDLTDEIVKDVNWTYEARVL |
| | SYPAGNVESTGSAGEPLYENSPEFT |
| | PYLETNLGQPTIQSFEQVGTKVNVT |
| | VEDERTLVRRNNTFLSLRDVFGKDL |
| | IYTLYYWKSSSSGKKTAKTNTNEFL |
| | IDVDKGENYCFSVQAVIPSRTVNRK |
| | STDSPVECMGQEKGEFRE |
| | (SEQ ID NO: 840) |
| h1-38_r | AGTPPGKAFNLTWISTDFKTILEWQ |
| | PKPTNYTYTVQISTKSGDWKSKCFY |
| | TTDTECDLTDEIVKDVKQTYLARVF |
| | SYPAGNVESTGSAGEPLYENSPEFT |
| | PYLETNLGQPTIQSFEQVGTKVNVT |
| | VEDERTLVRRNNTFLSLRDVFGKDL |
| | IYTLYYWKSSSSGKKTAKTNTNEFL |
| | IDVDKGENYCFSVQAVIPSRTVNRK |
| | STDSPVECMGQEKGEFRE |
| | (SEQ ID NO: 841) |
| h39-77_r | SGTTNTVAAYNLTWKSTNFKTILEW |
| | EPKPVNQVYTVQISDRSRNWKYKCT |
| | GTTDTECDLTDEIVKDVNWTYEARV |
| | LSYPAGNVESTGSAGEPLYENSPEF |
| | TPYLETNLGQPTIQSFEQVGTKVNV |
| | TVEDERTLVRRNNTFLSLRDVFGKD |
| | LIYTLYYWKSSSSGKKTAKTNTNEF |
| | LIDVDKGENYCFSVQAVIPSRTVNR |
| | KSTDSPVECMGQEKGEFRE |
| | (SEQ ID NO: 842) |
| h78-107_r | SGTTNTVAAYNLTWKSTNFKTILEW |
| | EPKPVNQVYTVQISTKSGDWKSKCF |
| | YTTDTECDLTDEIVKDVKQTYLARV |
| | FSVPWRNSTHGTHGEEPPFTNARKF |
| | LPYRDTKLGQPTIQSFEQVGTKVNV |
| | TVEDERTLVRRNNTFLSLRDVFGKD |
| | LIYTLYYWKSSSSGKKTAKTNTNEF |
| | LIDVDKGENYCFSVQAVIPSRTVNR |
| | KSTDSPVECMGQEKGEFRE |
| | (SEQ ID NO: 843) |
| h78-107_r.v2 | SGTTNTVAAYNLTWKSTNFKTILEW |
| | EPKPVNQVYTVQISTKSGDWKSKCF |
| | YTTDTECDLTDEIVKDVKQTYLARV |
| | FSVPWRNSTHGKETLFGTHGEEPPF |
| | TNARKFLPYRDTKLGQPTIQSFEQV |
| | GTKVNVTVEDERTLVRRNNTFLSLR |
| | DVFGKDLIYTLYYWKSSSSGKKTAK |
| | TNTNEFLIDVDKGENYCFSVQAVIP |
| | SRTVNRKSTDSPVECMGQEKGEFRE |
| | (SEQ ID NO: 844) |
| h78-93_r | SGTTNTVAAYNLTWKSTNFKTILEW |
| | EPKPVNQVYTVQISTKSGDWKSKCF |
| | YTTDTECDLTDEIVKDVKQTYLARV |
| | FSVPWRNSTHGKETLFGTHGEEPPY |
| | ENSPEFTPYLETNLGQPTIQSFEQV |
| | GTKVNVTVEDERTLVRRNNTFLSLR |
| | DVFGKDLIYTLYYWKSSSSGKKTAK |
| | TNTNEFLIDVDKGENYCFSVQAVIP |
| | SRTVNRKSTDSPVECMGQEKGEFRE |
| | (SEQ ID NO: 845) |
| h94-107_r | SGTTNTVAAYNLTWKSTNFKTILEW |
| | EPKPVNQVYTVQISTKSGDWKSKCF |
| | YTTDTECDLTDEIVKDVKQTYLARV |
| | FSYPAGNVESTGSAGEPLFTNARKF |
| | LPYRDTKLGQPTIQSFEQVGTKVNV |
| | TVEDERTLVRRNNTFLSLRDVFGKD |
| | LIYTLYYWKSSSSGKKTAKTNTNEF |
| | LIDVDKGENYCFSVQAVIPSRTVNR |
| | KSTDSPVECMGQEKGEFRE |
| | (SEQ ID NO: 846) |
| h108-219_r | SGTTNTVAAYNLTWKSTNFKTILEW |
| | EPKPVNQVYTVQISTKSGDWKSKCF |
| | YTTDTECDLTDEIVKDVKQTYLARV |
| | FSYPAGNVESTGSAGEPLYENSPEF |
| | TPYLETNIGQPVIQKYEQGGTKLKV |
| | TVKDSFTLVRKNGTFLTLRQVFGND |
| | LGYILTYRKDSSTGRKTNTTHTNEF |
| | LIDVEKGVSYCFFAQAVIFSRKTNH |
| | KSPESITKCTEQWKSVLGE |
| | (SEQ ID NO: 847) |
| h108-158_r | SGTTNTVAAYNLTWKSTNFKTILEW |
| | EPKPVNQVYTVQISTKSGDWKSKCF |
| | YTTDTECDLTDEIVKDVKQTYLARV |
| | FSYPAGNVESTGSAGEPLYENSPEF |
| | TPYLETNIGQPVIQKYEQGGTKLKV |
| | TVKDSFTLVRKNGTFLTLRQVFGND |
| | LGYILTYRKSSSSGKKTAKTNTNEF |
| | LIDVDKGENYCFSVQAVIPSRTVNR |
| | KSTDSPVECMGQEKGEFRE |
| | (SEQ ID NO: 848) |
| h108-132_r | SGTTNTVAAYNLTWKSTNFKTILEW |
| | EPKPVNQVYTVQISTKSGDWKSKCF |
| | YTTDTECDLTDEIVKDVKQTYLARV |
| | FSYPAGNVESTGSAGEPLYENSPEF |
| | TPYLETNIGQPVIQKYEQGGTKLKV |
| | TVKDSFTLVRRNNTFLSLRDVFGKD |
| | LIYTLYYWKSSSSGKKTAKTNTNEF |
| | LIDVDKGENYCFSVQAVIPSRTVNR |
| | KSTDSPVECMGQEKGEFRE |
| | (SEQ ID NO: 849) |
| h133-158_r | SGTTNTVAAYNLTWKSTNFKTILEW |
| | EPKPVNQVYTVQISTKSGDWKSKCF |
| | YTTDTECDLTDEIVKDVKQTYLARV |
| | FSYPAGNVESTGSAGEPLYENSPEF |
| | TPYLETNLGQPTIQSFEQVGTKVNV |
| | TVEDERTLVRKNGTFLTLRQVFGND |
| | LGYILTYRKSSSSGKKTAKTNTNEF |
| | LIDVDKGENYCFSVQAVIPSRTVNR |
| | KSTDSPVECMGQEKGEFRE |
| | (SEQ ID NO: 850) |
| h133-145_r | SGTTNTVAAYNLTWKSTNFKTILEW |
| | EPKPVNQVYTVQISTKSGDWKSKCF |
| | YTTDTECDLTDEIVKDVKQTYLARV |
| | FSYPAGNVESTGSAGEPLYENSPEF |
| | TPYLETNLGQPTIQSFEQVGTKVNV |
| | TVEDERTLVRKNGTFLTLRQVFGKD |
| | LIYTLYYWKSSSSGKKTAKTNTNEF |
| | LIDVDKGENYCFSVQAVIPSRTVNR |
| | KSTDSPVECMGQEKGEFRE |
| | (SEQ ID NO: 851) |
| h133-139_r | SGTTNTVAAYNLTWKSTNFKTILEW |
| | EPKPVNQVYTVQISTKSGDWKSKCF |
| | YTTDTECDLTDEIVKDVKQTYLARV |
| | FSYPAGNVESTGSAGEPLYENSPEF |
| | TPYLETNLGQPTIQSFEQVGTKVNV |
| | TVEDERTLVRKNGTFLSLRDVFGKD |
| | LIYTLYYWKSSSSGKKTAKTNTNEF |
| | LIDVDKGENYCFSVQAVIPSRTVNR |
| | KSTDSPVECMGQEKGEFRE |
| | (SEQ ID NO: 852) |
| h140-145_r | SGTTNTVAAYNLTWKSTNFKTILEW |
| | EPKPVNQVYTVQISTKSGDWKSKCF |
| | YTTDTECDLTDEIVKDVKQTYLARV |
| | FSYPAGNVESTGSAGEPLYENSPEF |
| | TPYLETNLGQPTIQSFEQVGTKVNV |
| | TVEDERTLVRRNNTFLTLRQVFGKD |
| | LIYTLYYWKSSSSGKKTAKTNTNEF |
| | LIDVDKGENYCFSVQAVIPSRTVNR |
| | KSTDSPVECMGQEKGEFRE |
| | (SEQ ID NO: 853) |
| h146-158_r | SGTTNTVAAYNLTWKSTNFKTILEW |
| | EPKPVNQVYTVQISTKSGDWKSKCF |
| | YTTDTECDLTDEIVKDVKQTYLARV |
| | FSYPAGNVESTGSAGEPLYENSPEF |
| | TPYLETNLGQPTIQSFEQVGTKVNV |
| | TVEDERTLVRRNNTFLSLRDVFGND |
| | LGYILTYRKSSSSGKKTAKTNTNEF |
| | LIDVDKGENYCFSVQAVIPSRTVNR |
| | KSTDSPVECMGQEKGEFRE |
| | (SEQ ID NO: 854) |
| h146-151_r | SGTTNTVAAYNLTWKSTNFKTILEW |
| | EPKPVNQVYTVQISTKSGDWKSKCF |
| | YTTDTECDLTDEIVKDVKQTYLARV |
| | FSYPAGNVESTGSAGEPLYENSPEF |
| | TPYLETNLGQPTIQSFEQVGTKVNV |
| | TVEDERTLVRRNNTFLSLRDVFGND |
| | LIYTLYYWKSSSSGKKTAKTNTNEF |
| | LIDVDKGENYCFSVQAVIPSRTVNR |
| | KSTDSPVECMGQEKGEFRE |
| | (SEQ ID NO: 855) |
| h152-158_r | SGTTNTVAAYNLTWKSTNFKTILEW |
| | EPKPVNQVYTVQISTKSGDWKSKCF |
| | YTTDTECDLTDEIVKDVKQTYLARV |
| | FSYPAGNVESTGSAGEPLYENSPEF |
| | TPYLETNLGQPTIQSFEQVGTKVNV |
| | TVEDERTLVRRNNTFLSLRDVFGKD |
| | LGYILTYRKSSSSGKKTAKTNTNEF |
| | LIDVDKGENYCFSVQAVIPSRTVNR |
| | KSTDSPVECMGQEKGEFRE |
| | (SEQ ID NO: 856) |
| h159-219_r | SGTTNTVAAYNLTWKSTNFKTILEW |
| | EPKPVNQVYTVQISTKSGDWKSKCF |
| | YTTDTECDLTDEIVKDVKQTYLARV |
| | FSYPAGNVESTGSAGEPLYENSPEF |
| | TPYLETNLGQPTIQSFEQVGTKVNV |
| | TVEDERTLVRRNNTFLSLRDVFGKD |
| | LIYTLYYWKDSSTGRKTNTTHTNEF |
| | LIDVEKGVSYCFFAQAVIFSRKTNH |
| | KSPESITKCTEQWKSVLGE |
| | (SEQ ID NO: 857) |
| h159-189_r | SGTTNTVAAYNLTWKSTNFKTILEW |
| | EPKPVNQVYTVQISTKSGDWKSKCF |
| | YTTDTECDLTDEIVKDVKQTYLARV |
| | FSYPAGNVESTGSAGEPLYENSPEF |
| | TPYLETNLGQPTIQSFEQVGTKVNV |
| | TVEDERTLVRRNNTFLSLRDVFGKD |
| | LIYTLYYWKDSSTGRKTNTTHTNEF |
| | LIDVEKGVSYCFFAQAVIPSRTVNR |
| | KSTDSPVECMGQEKGEFRE |
| | (SEQ ID NO: 858) |
| h159-174_r | SGTTNTVAAYNLTWKSTNFKTILEW |
| | EPKPVNQVYTVQISTKSGDWKSKCF |
| | YTTDTECDLTDEIVKDVKQTYLARV |
| | FSYPAGNVESTGSAGEPLYENSPEF |
| | TPYLETNLGQPTIQSFEQVGTKVNV |
| | TVEDERTLVRRNNTFLSLRDVFGKD |
| | LIYTLYYWKDSSTGRKTNTTHTNEF |
| | LIDVDKGENYCFSVQAVIPSRTVNR |
| | KSTDSPVECMGQEKGEFRE |
| | (SEQ ID NO: 859) |
| h159-166_r | SGTTNTVAAYNLTWKSTNFKTILEW |
| | EPKPVNQVYTVQISTKSGDWKSKCF |
| | YTTDTECDLTDEIVKDVKQTYLARV |
| | FSYPAGNVESTGSAGEPLYENSPEF |
| | TPYLETNLGQPTIQSFEQVGTKVNV |
| | TVEDERTLVRRNNTFLSLRDVFGKD |
| | LIYTLYYWKDSSTGRKTAKTNTNEF |
| | LIDVDKGENYCFSVQAVIPSRTVNR |
| | KSTDSPVECMGQEKGEFRE |
| | (SEQ ID NO: 860) |
| h167-174_r | SGTTNTVAAYNLTWKSTNFKTILEW |
| | EPKPVNQVYTVQISTKSGDWKSKCF |
| | YTTDTECDLTDEIVKDVKQTYLARV |
| | FSYPAGNVESTGSAGEPLYENSPEF |
| | TPYLETNLGQPTIQSFEQVGTKVNV |
| | TVEDERTLVRRNNTFLSLRDVFGKD |
| | LIYTLYYWKSSSSGKKTNTTHTNEF |
| | LIDVDKGENYCFSVQAVIPSRTVNR |
| | KSTDSPVECMGQEKGEFRE |
| | (SEQ ID NO: 861) |
| h175-189_r | SGTTNTVAAYNLTWKSTNFKTILEW |
| | EPKPVNQVYTVQISTKSGDWKSKCF |
| | YTTDTECDLTDEIVKDVKQTYLARV |
| | FSYPAGNVESTGSAGEPLYENSPEF |
| | TPYLETNLGQPTIQSFEQVGTKVNV |
| | TVEDERTLVRRNNTFLSLRDVFGKD |
| | LIYTLYYWKSSSSGKKTAKTNTNEF |
| | LIDVEKGVSYCFFAQAVIPSRTVNR |
| | KSTDSPVECMGQEKGEFRE |
| | (SEQ ID NO: 862) |
| h190-219_r | SGTTNTVAAYNLTWKSTNFKTILEW |
| | EPKPVNQVYTVQISTKSGDWKSKCF |
| | YTTDTECDLTDEIVKDVKQTYLARV |
| | FSYPAGNVESTGSAGEPLYENSPEF |
| | TPYLETNLGQPTIQSFEQVGTKVNV |
| | TVEDERTLVRRNNTFLSLRDVFGKD |
| | LIYTLYYWKSSSSGKKTAKTNTNEF |
| | LIDVDKGENYCFSVQAVIFSRKTNH |
| | KSPESITKCTEQWKSVLGE |
| | (SEQ ID NO: 863) |
| hTF_K68N | SGTTNTVAAYNLTWKSTNFKTILEW |
| | EPKPVNQVYTVQISTKSGDWKSKCF |
| | YTTDTECDLTDEIVKDVNQTYLARV |
| | FSYPAGNVESTGSAGEPLYENSPEF |
| | TPYLETNLGQPTIQSFEQVGTKVNV |
| | TVEDERTLVRRNNTFLSLRDVFGKD |
| | LIYTLYYWKSSSSGKKTAKTNTNEF |
| | LIDVDKGENYCFSVQAVIPSRTVNR |
| | KSTDSPVECMGQEKGEFRE |
| | (SEQ ID NO: 865) |
| hTF_K149N | SGTTNTVAAYNLTWKSTNFKTILEW |
| | EPKPVNQVYTVQISTKSGDWKSKCF |
| | YTTDTECDLTDEIVKDVKQTYLARV |
| | FSYPAGNVESTGSAGEPLYENSPEF |
| | TPYLETNLGQPTIQSFEQVGTKVNV |
| | TVEDERTLVRRNNTFLSLRDVFGND |
| | LIYTLYYWKSSSSGKKTAKTNTNEF |
| | LIDVDKGENYCFSVQAVIPSRTVNR |
| | KSTDSPVECMGQEKGEFRE |
| | (SEQ ID NO: 866) |
| hTF_N171H_T197K | SGTTNTVAAYNLTWKSTNFKTILEW |
| | EPKPVNQVYTVQISTKSGDWKSKCF |
| | YTTDTECDLTDEIVKDVKQTYLARV |
| | FSYPAGNVESTGSAGEPLYENSPEF |
| | TPYLETNLGQPTIQSFEQVGTKVNV |
| | TVEDERTLVRRNNTFLSLRDVFGKD |
| | LIYTLYYWKSSSSGKKTAKTHTNEF |
| | LIDVDKGENYCFSVQAVIPSRKVNR |
| | KSTDSPVECMGQEKGEFRE |
| | (SEQ ID NO: 867) |
| r141-194_h | AGTPPGKAFNLTWISTDFKTILEWQ |
| | PKPTNYTYTVQISDRSRNWKYKCTG |
| | TTDTECDLTDEIVKDVNWTYEARVL |
| | SVPWRNSTHOKETLFGTHGEEPPFT |
| | NARKFLPYRDTKIGQPVIQKYEQGG |
| | TKLKVTVKDSFTLVRRNNTFLSLRD |
| | VFGKDLIYTLYYWKSSSSGKKTAKT |
| | NTNEFLIDVDKGENYCFSVQAVIFS |
| | RKTNHKSPESITKCTEQWKSVLGE |
| | (SEQ ID NO: 864) |

TABLE 57
Variable region sequence consensus

| | VH Domain | VL Domain |
|---|---|---|
| | Consensus | Consensus |
| Group | (SEQ ID NO) | (SEQ ID NO) |
| Lineage | QVQLVQSGAEVKKP | DIQMTQSPSTLSAS |
| 25A | GASVKVSCKASGYT | VGDRVTITCx[R/Q] |
| | FDx[V/A]YGISWV | ASx[Q/E]SIx |
| | RQAPGQGLEWMGWI | [S/N]x[S/N\|WLA |
| | APYx[N/S]GNTNY | WYQQKPGKAPKLLI |
| | AQKLQGRVTMTTDT | YKAx[S/Y]x[S/N] |
| | STSTAYMELRSLRS | LEx\|S/Y]GVPSRFS |
| | DDTAVYYCARDAGT | GSGSGTEFTLTISS |
| | YSPFGYGMDVWGQG | LQPDDFATYYCQx |
| | TTVTVSS | [Q/L]FQx[S/K] |
| | (SEQ ID | LPPFTFGGGTKV |
| | NO: 868) | EIK |
| | | (SEQ ID NO: 869) |
| Lineage | QVQLVQSGAEVKKP | DIQMTQSPSTLSAS |
| 25G | GASVKVSCKASGYT | VGDRVTITCx[R/Q] |
| | FRSYGISWVRQAPG | ASx[Q/H)SIx[S/D] |
| | QGLEWMGWVAPYx | SWLAWYQQKPGKA |
| | [N/S]GNTNYAQKL | PKLLIYx\|K/S]ASx |
| | QGRVTMTT | [S/Y]LESGVPSRFSG |
| | DTSTSTAYMELRSL | SGSGTEFTLTISSLQP |
| | RSDDTAVYYCARDA | DDFATYYCQx[Q/L/R] |
| | GTYSPYGYGMDVWG | FQSLPPFTFGGGT |
| | QGTTVTVSS | KVEIK |
| | (SEQ ID | (SEQ ID NO: 871) |
| | NO: 870) | |

TABLE 58
Consensus CDRs

| | | Antibody Group | |
|---|---|---|---|
| | | Lineage 25A | Lineage 25G |
| VH CDR | VH CDR1 | GYTFDx[V/ | GYTFR |
| Seq.* | | A]YGIS | SYGIS |
| | | (SEQ ID | (SEQ ID |
| | | NO: 872) | NO: 878) |
| | VH CDR2 | WIAPYx | WVAPYx |
| | | [N/S] | [N/S]GN |
| | | GNTNYA | TNYAQK |
| | | QKLQG | LQG |
| | | (SEQ ID | (SEQ ID |
| | | NO: 873) | NO: 879) |
| | VH CDR3 | DAGTYSPF | DAGTYSP |
| | | GYGMDV | YGYGMDV |
| | | (SEQ ID | (SEQ ID |
| | | NO: 874) | NO: 880) |
| VL CDR | VL CDR1 | x[R/Q]ASx | x[R/Q]AS |
| Seq.* | | [Q/E]SIx | x[Q/H]S |
| | | [S/N]x[S/ | Ix[S/D] |
| | | N]WLA | SWLA |
| | | (SEQ ID | (SEQ ID |
| | | NO: 875) | NO: 881) |
| | VL CDR2 | KAx[S/Y] | x[K/S]AS |
| | | x[S/N]LE | x[S/Y]LES |
| | | x[S/Y] | (SEQ ID |
| | | (SEQ ID | NO: 882) |
| | | NO: 876) | |
| | VL CDR3 | Qx[Q/L]F | Qx[Q/L/R] |
| | | Qx[S/K]L | FQSLPPFT |
| | | PPFT | (SEQ ID |
| | | (SEQ ID | NO: 883) |
| | | NO: 877) | |

*Exemplary CDR sequences encompass amino acids as determined by Kabat & Chothia

1-503. (canceled)

504. An antibody which binds to the extracellular domain of human Tissue Factor (TF), wherein the antibody comprises
(1) a VH-CDR1, a VH-CDR2, and a VH-CDR3 comprising the amino acid sequences of the VH-CDR1, the VH-CDR2, and the VH-CDR3, respectively, as set forth in a VH comprising the amino acid sequence of SEQ ID NO: 37, and a VL-CDR1, a VL-CDR2, and a VL-CDR3 comprising the amino acid sequences of the VL-CDR1, the VL-CDR2, and the VL-CDR3, respectively, as set forth in a VL comprising the amino acid sequence of SEQ ID NO: 38;
(2) a VH-CDR1, a VH-CDR2, and a VH-CDR3 comprising the amino acid sequences of the VH-CDR1, the VH-CDR2, and the VH-CDR3, respectively, as set forth in a VH comprising the amino acid sequence of SEQ ID NO: 75, and a VL-CDR1, a VL-CDR2, and a VL-CDR3 comprising the amino acid sequences of the VL-CDR1, the VL-CDR2, and the VL-CDR3, respectively, as set forth in a VL comprising the amino acid sequence of SEQ ID NO: 76;
(3) a VH-CDR1, a VH-CDR2, and a VH-CDR3 comprising the amino acid sequences of the VH-CDR1, the VH-CDR2, and the VH-CDR3, respectively, as set forth in a VH comprising the amino acid sequence of SEQ ID NO: 113, and a VL-CDR1, a VL-CDR2, and a VL-CDR3 comprising the amino acid sequences of the VL-CDR1, the VL-CDR2, and the VL-CDR3, respectively, as set forth in a VL comprising the amino acid sequence of SEQ ID NO: 114;
(4) a VH-CDR1, a VH-CDR2, and a VH-CDR3 comprising the amino acid sequences of the VH-CDR1, the VH-CDR2, and the VH-CDR3, respectively, as set forth in a VH comprising the amino acid sequence of SEQ ID NO: 189, and a VL-CDR1, a VL-CDR2, and a VL-CDR3 comprising the amino acid sequences of the VL-CDR1, the VL-CDR2, and the VL-CDR3, respectively, as set forth in a VL comprising the amino acid sequence of SEQ ID NO: 190;
(5) a VH-CDR1, a VH-CDR2, and a VH-CDR3 comprising the amino acid sequences of the VH-CDR1, the VH-CDR2, and the VH-CDR3, respectively, as set forth in a VH comprising the amino acid sequence of SEQ ID NO: 227, and a VL-CDR1, a VL-CDR2, and a VL-CDR3 comprising the amino acid sequences of the VL-CDR1, the VL-CDR2, and the VL-CDR3, respectively, as set forth in a VL comprising the amino acid sequence of SEQ ID NO: 228;
(6) a VH-CDR1, a VH-CDR2, and a VH-CDR3 comprising the amino acid sequences of the VH-CDR1, the VH-CDR2, and the VH-CDR3, respectively, as set forth in a VH comprising the amino acid sequence of SEQ ID NO: 265, and a VL-CDR1, a VL-CDR2, and a VL-CDR3 comprising the amino acid sequences of the VL-CDR1, the VL-CDR2, and the VL-CDR3, respectively, as set forth in a VL comprising the amino acid sequence of SEQ ID NO: 266;
(7) a VH-CDR1, a VH-CDR2, and a VH-CDR3 comprising the amino acid sequences of the VH-CDR1, the VH-CDR2, and the VH-CDR3, respectively, as set forth in a VH comprising the amino acid sequence of SEQ ID NO: 303, and a VL-CDR1, a VL-CDR2, and a VL-CDR3 comprising the amino acid sequences of the VL-CDR1, the VL-CDR2, and the VL-CDR3, respectively, as set forth in a VL comprising the amino acid sequence of SEQ ID NO: 304;
(8) a VH-CDR1, a VH-CDR2, and a VH-CDR3 comprising the amino acid sequences of the VH-CDR1, the VH-CDR2, and the VH-CDR3, respectively, as set forth in a VH comprising the amino acid sequence of SEQ ID NO: 341, and a VL-CDR1, a VL-CDR2, and a VL-CDR3 comprising the amino acid sequences of the VL-CDR1, the VL-CDR2, and the VL-CDR3, respectively, as set forth in a VL comprising the amino acid sequence of SEQ ID NO: 342;
(9) a VH-CDR1, a VH-CDR2, and a VH-CDR3 comprising the amino acid sequences of the VH-CDR1, the VH-CDR2, and the VH-CDR3, respectively, as set forth in a VH comprising the amino acid sequence of SEQ ID NO: 379, and a VL-CDR1, a VL-CDR2, and a VL-CDR3 comprising the amino acid sequences of the VL-CDR1, the VL-CDR2, and the VL-CDR3, respectively, as set forth in a VL comprising the amino acid sequence of SEQ ID NO: 380;
(10) a VH-CDR1, a VH-CDR2, and a VH-CDR3 comprising the amino acid sequences of the VH-CDR1, the VH-CDR2, and the VH-CDR3, respectively, as set forth in a VH comprising the amino acid sequence of SEQ ID NO: 417, and a VL-CDR1, a VL-CDR2, and a VL-CDR3 comprising the amino acid sequences of the VL-CDR1, the VL-CDR2, and the VL-CDR3, respectively, as set forth in a VL comprising the amino acid sequence of SEQ ID NO: 418;
(11) a VH-CDR1, a VH-CDR2, and a VH-CDR3 comprising the amino acid sequences of the VH-CDR1, the VH-CDR2, and the VH-CDR3, respectively, as set forth in a VH comprising the amino acid sequence of SEQ ID NO: 455, and a VL-CDR1, a VL-CDR2, and a VL-CDR3 comprising the amino acid sequences of the VL-CDR1, the VL-CDR2, and the VL-CDR3, respectively, as set forth in a VL comprising the amino acid sequence of SEQ ID NO: 456;
(12) a VH-CDR1, a VH-CDR2, and a VH-CDR3 comprising the amino acid sequences of the VH-CDR1, the VH-CDR2, and the VH-CDR3, respectively, as set forth in a VH comprising the amino acid sequence of SEQ ID NO: 493, and a VL-CDR1, a VL-CDR2, and a VL-CDR3 comprising the amino acid sequences of the VL-CDR1, the VL-CDR2, and the VL-CDR3, respectively, as set forth in a VL comprising the amino acid sequence of SEQ ID NO: 494;
(13) a VH-CDR1, a VH-CDR2, and a VH-CDR3 comprising the amino acid sequences of the VH-CDR1, the VH-CDR2, and the VH-CDR3, respectively, as set forth in a VH comprising the amino acid sequence of SEQ ID NO: 531, and a VL-CDR1, a VL-CDR2, and a VL-CDR3 comprising the amino acid sequences of the VL-CDR1, the VL-CDR2, and the VL-CDR3, respectively, as set forth in a VL comprising the amino acid sequence of SEQ ID NO: 532;
(14) a VH-CDR1, a VH-CDR2, and a VH-CDR3 comprising the amino acid sequences of the VH-CDR1, the VH-CDR2, and the VH-CDR3, respectively, as set forth in a VH comprising the amino acid sequence of SEQ ID NO: 569, and a VL-CDR1, a VL-CDR2, and a VL-CDR3 comprising the amino acid sequences of the VL-CDR1, the VL-CDR2, and the VL-CDR3, respectively, as set forth in a VL comprising the amino acid sequence of SEQ ID NO: 570;
(15) a VH-CDR1, a VH-CDR2, and a VH-CDR3 comprising the amino acid sequences of the VH-CDR1, the VH-CDR2, and the VH-CDR3, respectively, as set forth in a VH comprising the amino acid sequence of SEQ ID NO: 607, and a VL-CDR1, a VL-CDR2, and a VL-CDR3 comprising the amino acid sequences of the VL-CDR1, the VL-CDR2, and the VL-CDR3, respectively, as set forth in a VL comprising the amino acid sequence of SEQ ID NO: 608;
(16) a VH-CDR1, a VH-CDR2, and a VH-CDR3 comprising the amino acid sequences of the VH-CDR1, the VH-CDR2, and the VH-CDR3, respectively, as set forth in a VH comprising the amino acid sequence of SEQ ID NO: 645, and a VL-CDR1, a VL-CDR2, and a VL-CDR3 comprising the amino acid sequences of the VL-CDR1, the VL-CDR2, and the VL-CDR3, respectively, as set forth in a VL comprising the amino acid sequence of SEQ ID NO: 646;
(17) a VH-CDR1, a VH-CDR2, and a VH-CDR3 comprising the amino acid sequences of the VH-CDR1, the VH-CDR2, and the VH-CDR3, respectively, as set forth in a VH comprising the amino acid sequence of SEQ ID NO: 683, and a VL-CDR1, a VL-CDR2, and a VL-CDR3 comprising the amino acid sequences of the VL-CDR1, the VL-CDR2, and the VL-CDR3, respectively, as set forth in a VL comprising the amino acid sequence of SEQ ID NO: 684;
(18) a VH-CDR1, a VH-CDR2, and a VH-CDR3 comprising the amino acid sequences of the VH-CDR1, the VH-CDR2, and the VH-CDR3, respectively, as set forth in a VH comprising the amino acid sequence of SEQ ID NO: 721, and a VL-CDR1, a VL-CDR2, and a VL-CDR3 comprising the amino acid sequences of the VL-CDR1, the VL-CDR2, and the VL-CDR3, respectively, as set forth in a VL comprising the amino acid sequence of SEQ ID NO: 722; or
(19) a VH-CDR1, a VH-CDR2, and a VH-CDR3 comprising the amino acid sequences of the VH-CDR1, the VH-CDR2, and the VH-CDR3, respectively, as set forth in a VH comprising the amino acid sequence of SEQ ID NO: 759, and a VL-CDR1, a VL-CDR2, and a VL-CDR3 comprising the amino acid sequences of the VL-CDR1, the VL-CDR2, and the VL-CDR3, respectively, as set forth in a VL comprising the amino acid sequence of SEQ ID NO: 760.

505. The antibody of claim 504, wherein the VH-CDR1, the VH-CDR2, the VH-CDR3, the VL-CDR1, the VL-CDR2, and the VL-CDR3 are determined according to Kabat numbering scheme, Chothia numbering scheme, AbM numbering scheme, Contact numbering scheme, IMGT numbering scheme, or a combination thereof.

506. The antibody of claim 504, wherein
(1) (i) the VH-CDR1 comprises the amino acid sequence of SEQ ID NO: 1, the VH-CDR2 comprises the amino acid sequence of SEQ ID NO: 2, the VH-CDR3 comprises the amino acid sequence of SEQ ID NO: 3, the VL-CDR1 comprises the amino acid sequence of SEQ ID NO: 4, the VL-CDR2 comprises the amino acid sequence of SEQ ID NO: 5, and the VL-CDR3 comprises the amino acid sequence of SEQ ID NO: 6;
(ii) the VH-CDR1 comprises the amino acid sequence of SEQ ID NO: 7, the VH-CDR2 comprises the amino acid sequence of SEQ ID NO: 8, the VH-CDR3 comprises the amino acid sequence of SEQ ID NO: 9, the VL-CDR1 comprises the amino acid sequence of SEQ ID NO: 10, the VL-CDR2 comprises the amino acid sequence of SEQ ID NO: 11, and the VL-CDR3 comprises the amino acid sequence of SEQ ID NO: 12;
(iii) the VH-CDR1 comprises the amino acid sequence of SEQ ID NO: 13, the VH-CDR2 comprises the amino acid sequence of SEQ ID NO: 14, the VH-CDR3 comprises the amino acid sequence of SEQ ID NO: 15, the VL-CDR1 comprises the amino acid sequence of SEQ ID NO: 16, the VL-CDR2 comprises the amino acid sequence of KAS, and the VL-CDR3 comprises the amino acid sequence of SEQ ID NO: 18;
(iv) the VH-CDR1 comprises the amino acid sequence of SEQ ID NO: 19, the VH-CDR2 comprises the amino acid sequence of SEQ ID NO: 20, the VH-CDR3 comprises the amino acid sequence of SEQ ID NO: 21, the VL-CDR1 comprises the amino acid sequence of SEQ ID NO: 22, the VL-CDR2 comprises the amino acid sequence of SEQ ID NO: 23, and the VL-CDR3 comprises the amino acid sequence of SEQ ID NO: 24;
(v) the VH-CDR1 comprises the amino acid sequence of SEQ ID NO: 25, the VH-CDR2 comprises the amino acid sequence of SEQ ID NO: 26, the VH-CDR3 comprises the amino acid sequence of SEQ ID NO: 27, the VL-CDR1 comprises the amino acid sequence of SEQ ID NO: 28, the VL-CDR2 comprises the amino acid sequence of SEQ ID NO: 29, and the VL-CDR3 comprises the amino acid sequence of SEQ ID NO: 30; or
(vi) the VH-CDR1 comprises the amino acid sequence of SEQ ID NO: 31, the VH-CDR2 comprises the amino acid sequence of SEQ ID NO: 32, the VH-CDR3 comprises the amino acid sequence of SEQ ID NO: 33, the VL-CDR1 comprises the amino acid sequence of SEQ ID NO: 34, the VL-CDR2 comprises the amino acid sequence of KAS, and the VL-CDR3 comprises the amino acid sequence of SEQ ID NO: 36;
(2) (i) the VH-CDR1 comprises the amino acid sequence of SEQ ID NO: 39, the VH-CDR2 comprises the amino acid sequence of SEQ ID NO: 40, the VH-CDR3 comprises the amino acid sequence of SEQ ID NO: 41, the VL-CDR1 comprises the amino acid sequence of SEQ ID NO: 42, the VL-CDR2 comprises the amino acid sequence of SEQ ID NO: 43, and the VL-CDR3 comprises the amino acid sequence of SEQ ID NO: 44;
(ii) the VH-CDR1 comprises the amino acid sequence of SEQ ID NO: 45, the VH-CDR2 comprises the amino acid sequence of SEQ ID NO: 46, the VH-CDR3 comprises the amino acid sequence of SEQ ID NO: 47, the VL-CDR1 comprises the amino acid sequence of SEQ ID NO: 48, the VL-CDR2 comprises the amino acid sequence of SEQ ID NO: 49, and the VL-CDR3 comprises the amino acid sequence of SEQ ID NO: 50;
(iii) the VH-CDR1 comprises the amino acid sequence of SEQ ID NO: 51, the VH-CDR2 comprises the amino acid sequence of SEQ ID NO: 52, the VH-CDR3 comprises the amino acid sequence of SEQ ID NO: 53, the VL-CDR1 comprises the amino acid sequence of SEQ ID NO: 54, the VL-CDR2 comprises the amino acid sequence of KAS, and the VL-CDR3 comprises the amino acid sequence of SEQ ID NO: 56;
(iv) the VH-CDR1 comprises the amino acid sequence of SEQ ID NO: 57, the VH-CDR2 comprises the amino acid sequence of SEQ ID NO: 58, the VH-CDR3 comprises the amino acid sequence of SEQ ID NO: 59, the VL-CDR1 comprises the amino acid sequence of SEQ ID NO: 60, the VL-CDR2 comprises the amino acid sequence of SEQ ID NO: 61, and the VL-CDR3 comprises the amino acid sequence of SEQ ID NO: 62;
(v) the VH-CDR1 comprises the amino acid sequence of SEQ ID NO: 63, the VH-CDR2 comprises the amino acid sequence of SEQ ID NO: 64, the VH-CDR3 comprises the amino acid sequence of SEQ ID NO: 65, the VL-CDR1 comprises the amino acid sequence of SEQ ID NO: 66, the VL-CDR2 comprises the amino acid sequence of SEQ ID NO: 67, and the VL-CDR3 comprises the amino acid sequence of SEQ ID NO: 68; or
(vi) the VH-CDR1 comprises the amino acid sequence of SEQ ID NO: 69, the VH-CDR2 comprises the amino acid sequence of SEQ ID NO: 70, the VH-CDR3 comprises the amino acid sequence of SEQ ID NO: 71, the VL-CDR1 comprises the amino acid sequence of SEQ ID NO: 72, the VL-CDR2 comprises the amino acid sequence of KAS, and the VL-CDR3 comprises the amino acid sequence of SEQ ID NO: 74;
(3) (i) the VH-CDR1 comprises the amino acid sequence of SEQ ID NO: 77, the VH-CDR2 comprises the amino acid sequence of SEQ ID NO: 78, the VH-CDR3 comprises the amino acid sequence of SEQ ID NO: 79, the VL-CDR1 comprises the amino acid sequence of SEQ ID NO: 80, the VL-CDR2 comprises the amino acid sequence of SEQ ID NO: 81, and the VL-CDR3 comprises the amino acid sequence of SEQ ID NO: 82;
(ii) the VH-CDR1 comprises the amino acid sequence of SEQ ID NO: 83, the VH-CDR2 comprises the amino acid sequence of SEQ ID NO: 84, the VH-CDR3 comprises the amino acid sequence of SEQ ID NO: 85, the VL-CDR1 comprises the amino acid sequence of SEQ ID NO: 86, the VL-CDR2 comprises the amino acid sequence of SEQ ID NO: 87, and the VL-CDR3 comprises the amino acid sequence of SEQ ID NO: 88;
(iii) the VH-CDR1 comprises the amino acid sequence of SEQ ID NO: 89, the VH-CDR2 comprises the amino acid sequence of SEQ ID NO: 90, the VH-CDR3 comprises the amino acid sequence of SEQ ID NO: 91, the VL-CDR1 comprises the amino acid sequence of SEQ ID NO: 92, the VL-CDR2 comprises the amino acid sequence of KAS, and the VL-CDR3 comprises the amino acid sequence of SEQ ID NO: 94;
(iv) the VH-CDR1 comprises the amino acid sequence of SEQ ID NO: 95, the VH-CDR2 comprises the amino acid sequence of SEQ ID NO: 96, the VH-CDR3 comprises the amino acid sequence of SEQ ID NO: 97, the VL-CDR1 comprises the amino acid sequence of SEQ ID NO: 98, the VL-CDR2 comprises the amino acid sequence of SEQ ID NO: 99, and the VL-CDR3 comprises the amino acid sequence of SEQ ID NO: 100;
(v) the VH-CDR1 comprises the amino acid sequence of SEQ ID NO: 101, the VH-CDR2 comprises the amino acid sequence of SEQ ID NO: 102, the VH-CDR3 comprises the amino acid sequence of SEQ ID NO: 103, the VL-CDR1 comprises the amino acid sequence of SEQ ID NO: 104, the VL-CDR2 comprises the amino acid sequence of SEQ ID NO: 105, and the VL-CDR3 comprises the amino acid sequence of SEQ ID NO: 106; or
(vi) the VH-CDR1 comprises the amino acid sequence of SEQ ID NO: 107, the VH-CDR2 comprises the amino acid sequence of SEQ ID NO: 108, the VH-CDR3 comprises the amino acid sequence of SEQ ID NO: 109, the VL-CDR1 comprises the amino acid sequence of SEQ ID NO: 110, the VL-CDR2 comprises the amino acid sequence of KAS, and the VL-CDR3 comprises the amino acid sequence of SEQ ID NO: 112;
(4) (i) the VH-CDR1 comprises the amino acid sequence of SEQ ID NO: 153, the VH-CDR2 comprises the amino acid sequence of SEQ ID NO: 154, the VH-CDR3 comprises the amino acid sequence of SEQ ID NO: 155, the VL-CDR1 comprises the amino acid sequence of SEQ ID NO: 156, the VL-CDR2 comprises the amino acid sequence of SEQ ID NO: 157, and the VL-CDR3 comprises the amino acid sequence of SEQ ID NO: 158;
(ii) the VH-CDR1 comprises the amino acid sequence of SEQ ID NO: 159, the VH-CDR2 comprises the amino acid sequence of SEQ ID NO: 160, the VH-CDR3 comprises the amino acid sequence of SEQ ID NO: 161, the VL-CDR1 comprises the amino acid sequence of SEQ ID NO: 162, the VL-CDR2 comprises the amino acid sequence of SEQ ID NO: 163, and the VL-CDR3 comprises the amino acid sequence of SEQ ID NO: 164;
(iii) the VH-CDR1 comprises the amino acid sequence of SEQ ID NO: 165, the VH-CDR2 comprises the amino acid sequence of SEQ ID NO: 166, the VH-CDR3 comprises the amino acid sequence of SEQ ID NO: 167, the VL-CDR1 comprises the amino acid sequence of SEQ ID NO: 168, the VL-CDR2 comprises the amino acid sequence of KAY, and the VL-CDR3 comprises the amino acid sequence of SEQ ID NO: 170;
(iv) the VH-CDR1 comprises the amino acid sequence of SEQ ID NO: 171, the VH-CDR2 comprises the amino acid sequence of SEQ ID NO: 172, the VH-CDR3 comprises the amino acid sequence of SEQ ID NO: 173, the VL-CDR1 comprises the amino acid sequence of SEQ ID NO: 174, the VL-CDR2 comprises the amino acid sequence of SEQ ID NO: 175, and the VL-CDR3 comprises the amino acid sequence of SEQ ID NO: 176;
(v) the VH-CDR1 comprises the amino acid sequence of SEQ ID NO: 177, the VH-CDR2 comprises the amino acid sequence of SEQ ID NO: 178, the VH-CDR3 comprises the amino acid sequence of SEQ ID NO: 179, the VL-CDR1 comprises the amino acid sequence of SEQ ID NO: 180, the VL-CDR2 comprises the amino acid sequence of SEQ ID NO: 181, and the VL-CDR3 comprises the amino acid sequence of SEQ ID NO: 182; or
(vi) the VH-CDR1 comprises the amino acid sequence of SEQ ID NO: 183, the VH-CDR2 comprises the amino acid sequence of SEQ ID NO: 184, the VH-CDR3 comprises the amino acid sequence of SEQ ID NO: 185, the VL-CDR1 comprises the amino acid sequence of SEQ ID NO: 186, the VL-CDR2 comprises the amino acid sequence of KAY, and the VL-CDR3 comprises the amino acid sequence of SEQ ID NO: 188;
(5) (i) the VH-CDR1 comprises the amino acid sequence of SEQ ID NO: 191, the VH-CDR2 comprises the amino acid sequence of SEQ ID NO: 192, the VH-CDR3 comprises the amino acid sequence of SEQ ID NO: 193, the VL-CDR1 comprises the amino acid sequence of SEQ ID NO: 194, the VL-CDR2 comprises the amino acid sequence of SEQ ID NO: 195, and the VL-CDR3 comprises the amino acid sequence of SEQ ID NO: 196;
(ii) the VH-CDR1 comprises the amino acid sequence of SEQ ID NO: 197, the VH-CDR2 comprises the amino acid sequence of SEQ ID NO: 198, the VH-CDR3 comprises the amino acid sequence of SEQ ID NO: 199, the VL-CDR1 comprises the amino acid sequence of SEQ ID NO: 200, the VL-CDR2 comprises the amino acid sequence of SEQ ID NO: 201, and the VL-CDR3 comprises the amino acid sequence of SEQ ID NO: 202;
(iii) the VH-CDR1 comprises the amino acid sequence of SEQ ID NO: 203, the VH-CDR2 comprises the amino acid sequence of SEQ ID NO: 204, the VH-CDR3 comprises the amino acid sequence of SEQ ID NO: 205, the VL-CDR1 comprises the amino acid sequence of SEQ ID NO: 206, the VL-CDR2 comprises the amino acid sequence of KAS, and the VL-CDR3 comprises the amino acid sequence of SEQ ID NO: 208;
(iv) the VH-CDR1 comprises the amino acid sequence of SEQ ID NO: 209, the VH-CDR2 comprises the amino acid sequence of SEQ ID NO: 210, the VH-CDR3 comprises the amino acid sequence of SEQ ID NO: 211, the VL-CDR1 comprises the amino acid sequence of SEQ ID NO: 212, the VL-CDR2 comprises the amino acid sequence of SEQ ID NO: 213, and the VL-CDR3 comprises the amino acid sequence of SEQ ID NO: 214;
(v) the VH-CDR1 comprises the amino acid sequence of SEQ ID NO: 215, the VH-CDR2 comprises the amino acid sequence of SEQ ID NO: 216, the VH-CDR3 comprises the amino acid sequence of SEQ ID NO: 217, the VL-CDR1 comprises the amino acid sequence of SEQ ID NO: 218, the VL-CDR2 comprises the amino acid sequence of SEQ ID NO: 219, and the VL-CDR3 comprises the amino acid sequence of SEQ ID NO: 220; or
(vi) the VH-CDR1 comprises the amino acid sequence of SEQ ID NO: 221, the VH-CDR2 comprises the amino acid sequence of SEQ ID NO: 222, the VH-CDR3 comprises the amino acid sequence of SEQ ID NO: 223, the VL-CDR1 comprises the amino acid sequence of SEQ ID NO: 224, the VL-CDR2 comprises the amino acid sequence of KAS, and the VL-CDR3 comprises the amino acid sequence of SEQ ID NO: 226;
(6) (i) the VH-CDR1 comprises the amino acid sequence of SEQ ID NO: 229, the VH-CDR2 comprises the amino acid sequence of SEQ ID NO: 230, the VH-CDR3 comprises the amino acid sequence of SEQ ID NO: 231, the VL-CDR1 comprises the amino acid sequence of SEQ ID NO: 232, the VL-CDR2 comprises the amino acid sequence of SEQ ID NO: 233, and the VL-CDR3 comprises the amino acid sequence of SEQ ID NO: 234;
(ii) the VH-CDR1 comprises the amino acid sequence of SEQ ID NO: 235, the VH-CDR2 comprises the amino acid sequence of SEQ ID NO: 236, the VH-CDR3 comprises the amino acid sequence of SEQ ID NO: 237, the VL-CDR1 comprises the amino acid sequence of SEQ ID NO: 238, the VL-CDR2 comprises the amino acid sequence of SEQ ID NO: 239, and the VL-CDR3 comprises the amino acid sequence of SEQ ID NO: 240;
(iii) the VH-CDR1 comprises the amino acid sequence of SEQ ID NO: 241, the VH-CDR2 comprises the amino acid sequence of SEQ ID NO: 242, the VH-CDR3 comprises the amino acid sequence of SEQ ID NO: 243, the VL-CDR1 comprises the amino acid sequence of SEQ ID NO: 244, the VL-CDR2 comprises the amino acid sequence of KAS, and the VL-CDR3 comprises the amino acid sequence of SEQ ID NO: 246;
(iv) the VH-CDR1 comprises the amino acid sequence of SEQ ID NO: 247, the VH-CDR2 comprises the amino acid sequence of SEQ ID NO: 248, the VH-CDR3 comprises the amino acid sequence of SEQ ID NO: 249, the VL-CDR1 comprises the amino acid sequence of SEQ ID NO: 250, the VL-CDR2 comprises the amino acid sequence of SEQ ID NO: 251, and the VL-CDR3 comprises the amino acid sequence of SEQ ID NO: 252;
(v) the VH-CDR1 comprises the amino acid sequence of SEQ ID NO: 253, the VH-CDR2 comprises the amino acid sequence of SEQ ID NO: 254, the VH-CDR3 comprises the amino acid sequence of SEQ ID NO: 255, the VL-CDR1 comprises the amino acid sequence of SEQ ID NO: 256, the VL-CDR2 comprises the amino acid sequence of SEQ ID NO: 257, and the VL-CDR3 comprises the amino acid sequence of SEQ ID NO: 258; or
(vi) the VH-CDR1 comprises the amino acid sequence of SEQ ID NO: 259, the VH-CDR2 comprises the amino acid sequence of SEQ ID NO: 260, the VH-CDR3 comprises the amino acid sequence of SEQ ID NO: 261, the VL-CDR1 comprises the amino acid sequence of SEQ ID NO: 262, the VL-CDR2 comprises the amino acid sequence of KAS, and the VL-CDR3 comprises the amino acid sequence of SEQ ID NO: 264;
(7) (i) the VH-CDR1 comprises the amino acid sequence of SEQ ID NO: 267, the VH-CDR2 comprises the amino acid sequence of SEQ ID NO: 268, the VH-CDR3 comprises the amino acid sequence of SEQ ID NO: 269, the VL-CDR1 comprises the amino acid sequence of SEQ ID NO: 270, the VL-CDR2 comprises the amino acid sequence of SEQ ID NO: 271, and the VL-CDR3 comprises the amino acid sequence of SEQ ID NO: 272;
(ii) the VH-CDR1 comprises the amino acid sequence of SEQ ID NO: 273, the VH-CDR2 comprises the amino acid sequence of SEQ ID NO: 274, the VH-CDR3 comprises the amino acid sequence of SEQ ID NO: 275, the VL-CDR1 comprises the amino acid sequence of SEQ ID NO: 276, the VL-CDR2 comprises the amino acid sequence of SEQ ID NO: 277, and the VL-CDR3 comprises the amino acid sequence of SEQ ID NO: 278;
(iii) the VH-CDR1 comprises the amino acid sequence of SEQ ID NO: 279, the VH-CDR2 comprises the amino acid sequence of SEQ ID NO: 280, the VH-CDR3 comprises the amino acid sequence of SEQ ID NO: 281, the VL-CDR1 comprises the amino acid sequence of SEQ ID NO: 282, the VL-CDR2 comprises the amino acid sequence of SAS, and the VL-CDR3 comprises the amino acid sequence of SEQ ID NO: 284;
(iv) the VH-CDR1 comprises the amino acid sequence of SEQ ID NO: 285, the VH-CDR2 comprises the amino acid sequence of SEQ ID NO: 286, the VH-CDR3 comprises the amino acid sequence of SEQ ID NO: 287, the VL-CDR1 comprises the amino acid sequence of SEQ ID NO: 288, the VL-CDR2 comprises the amino acid sequence of SEQ ID NO: 289, and the VL-CDR3 comprises the amino acid sequence of SEQ ID NO: 290;
(v) the VH-CDR1 comprises the amino acid sequence of SEQ ID NO: 291, the VH-CDR2 comprises the amino acid sequence of SEQ ID NO: 292, the VH-CDR3 comprises the amino acid sequence of SEQ ID NO: 293, the VL-CDR1 comprises the amino acid sequence of SEQ ID NO: 294, the VL-CDR2 comprises the amino acid sequence of SEQ ID NO: 295, and the VL-CDR3 comprises the amino acid sequence of SEQ ID NO: 296; or
(vi) the VH-CDR1 comprises the amino acid sequence of SEQ ID NO: 297, the VH-CDR2 comprises the amino acid sequence of SEQ ID NO: 298, the VH-CDR3 comprises the amino acid sequence of SEQ ID NO: 299, the VL-CDR1 comprises the amino acid sequence of SEQ ID NO: 300, the VL-CDR2 comprises the amino acid sequence of SAS, and the VL-CDR3 comprises the amino acid sequence of SEQ ID NO: 302;
(8) (i) the VH-CDR1 comprises the amino acid sequence of SEQ ID NO: 305, the VH-CDR2 comprises the amino acid sequence of SEQ ID NO: 306, the VH-CDR3 comprises the amino acid sequence of SEQ ID NO: 307, the VL-CDR1 comprises the amino acid sequence of SEQ ID NO: 308, the VL-CDR2 comprises the amino acid sequence of SEQ ID NO: 309, and the VL-CDR3 comprises the amino acid sequence of SEQ ID NO: 310;
(ii) the VH-CDR1 comprises the amino acid sequence of SEQ ID NO: 311, the VH-CDR2 comprises the amino acid sequence of SEQ ID NO: 312, the VH-CDR3 comprises the amino acid sequence of SEQ ID NO: 313, the VL-CDR1 comprises the amino acid sequence of SEQ ID NO: 314, the VL-CDR2 comprises the amino acid sequence of SEQ ID NO: 315, and the VL-CDR3 comprises the amino acid sequence of SEQ ID NO: 316;
(iii) the VH-CDR1 comprises the amino acid sequence of SEQ ID NO: 317, the VH-CDR2 comprises the amino acid sequence of SEQ ID NO: 318, the VH-CDR3 comprises the amino acid sequence of SEQ ID NO: 319, the VL-CDR1 comprises the amino acid sequence of SEQ ID NO: 320, the VL-CDR2 comprises the amino acid sequence of WAS, and the VL-CDR3 comprises the amino acid sequence of SEQ ID NO: 322;
(iv) the VH-CDR1 comprises the amino acid sequence of SEQ ID NO: 323, the VH-CDR2 comprises the amino acid sequence of SEQ ID NO: 324, the VH-CDR3 comprises the amino acid sequence of SEQ ID NO: 325, the VL-CDR1 comprises the amino acid sequence of SEQ ID NO: 326, the VL-CDR2 comprises the amino acid sequence of SEQ ID NO: 327, and the VL-CDR3 comprises the amino acid sequence of SEQ ID NO: 328;
(v) the VH-CDR1 comprises the amino acid sequence of SEQ ID NO: 329, the VH-CDR2 comprises the amino acid sequence of SEQ ID NO: 330, the VH-CDR3 comprises the amino acid sequence of SEQ ID NO: 331, the VL-CDR1 comprises the amino acid sequence of SEQ ID NO: 332, the VL-CDR2 comprises the amino acid sequence of SEQ ID NO: 333, and the VL-CDR3 comprises the amino acid sequence of SEQ ID NO: 334; or
(vi) the VH-CDR1 comprises the amino acid sequence of SEQ ID NO: 335, the VH-CDR2 comprises the amino acid sequence of SEQ ID NO: 336, the VH-CDR3 comprises the amino acid sequence of SEQ ID NO: 337, the VL-CDR1 comprises the amino acid sequence of SEQ ID NO: 338, the VL-CDR2 comprises the amino acid sequence of WAS, and the VL-CDR3 comprises the amino acid sequence of SEQ ID NO: 340;
(9) (i) the VH-CDR1 comprises the amino acid sequence of SEQ ID NO: 343, the VH-CDR2 comprises the amino acid sequence of SEQ ID NO: 344, the VH-CDR3 comprises the amino acid sequence of SEQ ID NO: 345, the VL-CDR1 comprises the amino acid sequence of SEQ ID NO: 346, the VL-CDR2 comprises the amino acid sequence of SEQ ID NO: 347, and the VL-CDR3 comprises the amino acid sequence of SEQ ID NO: 348;
(ii) the VH-CDR1 comprises the amino acid sequence of SEQ ID NO: 349, the VH-CDR2 comprises the amino acid sequence of SEQ ID NO: 350, the VH-CDR3 comprises the amino acid sequence of SEQ ID NO: 351, the VL-CDR1 comprises the amino acid sequence of SEQ ID NO: 352, the VL-CDR2 comprises the amino acid sequence of SEQ ID NO: 353, and the VL-CDR3 comprises the amino acid sequence of SEQ ID NO: 354;
(iii) the VH-CDR1 comprises the amino acid sequence of SEQ ID NO: 355, the VH-CDR2 comprises the amino acid sequence of SEQ ID NO: 356, the VH-CDR3 comprises the amino acid sequence of SEQ ID NO: 357, the VL-CDR1 comprises the amino acid sequence of SEQ ID NO: 358, the VL-CDR2 comprises the amino acid sequence of WAS, and the VL-CDR3 comprises the amino acid sequence of SEQ ID NO: 360;
(iv) the VH-CDR1 comprises the amino acid sequence of SEQ ID NO: 361, the VH-CDR2 comprises the amino acid sequence of SEQ ID NO: 362, the VH-CDR3 comprises the amino acid sequence of SEQ ID NO: 363, the VL-CDR1 comprises the amino acid sequence of SEQ ID NO: 364, the VL-CDR2 comprises the amino acid sequence of SEQ ID NO: 365, and the VL-CDR3 comprises the amino acid sequence of SEQ ID NO: 366;
(v) the VH-CDR1 comprises the amino acid sequence of SEQ ID NO: 367, the VH-CDR2 comprises the amino acid sequence of SEQ ID NO: 368, the VH-CDR3 comprises the amino acid sequence of SEQ ID NO: 369, the VL-CDR1 comprises the amino acid sequence of SEQ ID NO: 370, the VL-CDR2 comprises the amino acid sequence of SEQ ID NO: 371, and the VL-CDR3 comprises the amino acid sequence of SEQ ID NO: 372; or
(vi) the VH-CDR1 comprises the amino acid sequence of SEQ ID NO: 373, the VH-CDR2 comprises the amino acid sequence of SEQ ID NO: 374, the VH-CDR3 comprises the amino acid sequence of SEQ ID NO: 375, the VL-CDR1 comprises the amino acid sequence of SEQ ID NO: 376, the VL-CDR2 comprises the amino acid sequence of WAS, and the VL-CDR3 comprises the amino acid sequence of SEQ ID NO: 378;
(10) (i) the VH-CDR1 comprises the amino acid sequence of SEQ ID NO: 381, the VH-CDR2 comprises the amino acid sequence of SEQ ID NO: 382, the VH-CDR3 comprises the amino acid sequence of SEQ ID NO: 383, the VL-CDR1 comprises the amino acid sequence of SEQ ID NO: 384, the VL-CDR2 comprises the amino acid sequence of SEQ ID NO: 385, and the VL-CDR3 comprises the amino acid sequence of SEQ ID NO: 386;
(ii) the VH-CDR1 comprises the amino acid sequence of SEQ ID NO: 387, the VH-CDR2 comprises the amino acid sequence of SEQ ID NO: 388, the VH-CDR3 comprises the amino acid sequence of SEQ ID NO: 389, the VL-CDR1 comprises the amino acid sequence of SEQ ID NO: 390, the VL-CDR2 comprises the amino acid sequence of SEQ ID NO: 391, and the VL-CDR3 comprises the amino acid sequence of SEQ ID NO: 392;
(iii) the VH-CDR1 comprises the amino acid sequence of SEQ ID NO: 393, the VH-CDR2 comprises the amino acid sequence of SEQ ID NO: 394, the VH-CDR3 comprises the amino acid sequence of SEQ ID NO: 395, the VL-CDR1 comprises the amino acid sequence of SEQ ID NO: 396, the VL-CDR2 comprises the amino acid sequence of GAS, and the VL-CDR3 comprises the amino acid sequence of SEQ ID NO: 398;
(iv) the VH-CDR1 comprises the amino acid sequence of SEQ ID NO: 399, the VH-CDR2 comprises the amino acid sequence of SEQ ID NO: 400, the VH-CDR3 comprises the amino acid sequence of SEQ ID NO: 401, the VL-CDR1 comprises the amino acid sequence of SEQ ID NO: 402, the VL-CDR2 comprises the amino acid sequence of SEQ ID NO: 403, and the VL-CDR3 comprises the amino acid sequence of SEQ ID NO: 404;
(v) the VH-CDR1 comprises the amino acid sequence of SEQ ID NO: 405, the VH-CDR2 comprises the amino acid sequence of SEQ ID NO: 406, the VH-CDR3 comprises the amino acid sequence of SEQ ID NO: 407, the VL-CDR1 comprises the amino acid sequence of SEQ ID NO: 408, the VL-CDR2 comprises the amino acid sequence of SEQ ID NO: 409, and the VL-CDR3 comprises the amino acid sequence of SEQ ID NO: 410; or
(vi) the VH-CDR1 comprises the amino acid sequence of SEQ ID NO: 411, the VH-CDR2 comprises the amino acid sequence of SEQ ID NO: 412, the VH-CDR3 comprises the amino acid sequence of SEQ ID NO: 413, the VL-CDR1 comprises the amino acid sequence of SEQ ID NO: 414, the VL-CDR2 comprises the amino acid sequence of GAS, and the VL-CDR3 comprises the amino acid sequence of SEQ ID NO: 416;
(11) (i) the VH-CDR1 comprises the amino acid sequence of SEQ ID NO: 419, the VH-CDR2 comprises the amino acid sequence of SEQ ID NO: 420, the VH-CDR3 comprises the amino acid sequence of SEQ ID NO: 421, the VL-CDR1 comprises the amino acid sequence of SEQ ID NO: 422, the VL-CDR2 comprises the amino acid sequence of SEQ ID NO: 423, and the VL-CDR3 comprises the amino acid sequence of SEQ ID NO: 424;
(ii) the VH-CDR1 comprises the amino acid sequence of SEQ ID NO: 425, the VH-CDR2 comprises the amino acid sequence of SEQ ID NO: 426, the VH-CDR3 comprises the amino acid sequence of SEQ ID NO: 427, the VL-CDR1 comprises the amino acid sequence of SEQ ID NO: 428, the VL-CDR2 comprises the amino acid sequence of SEQ ID NO: 429, and the VL-CDR3 comprises the amino acid sequence of SEQ ID NO: 430;
(iii) the VH-CDR1 comprises the amino acid sequence of SEQ ID NO: 431, the VH-CDR2 comprises the amino acid sequence of SEQ ID NO: 432, the VH-CDR3 comprises the amino acid sequence of SEQ ID NO: 433, the VL-CDR1 comprises the amino acid sequence of SEQ ID NO: 434, the VL-CDR2 comprises the amino acid sequence of GAS, and the VL-CDR3 comprises the amino acid sequence of SEQ ID NO: 436;
(iv) the VH-CDR1 comprises the amino acid sequence of SEQ ID NO: 437, the VH-CDR2 comprises the amino acid sequence of SEQ ID NO: 438, the VH-CDR3 comprises the amino acid sequence of SEQ ID NO: 439, the VL-CDR1 comprises the amino acid sequence of SEQ ID NO: 440, the VL-CDR2 comprises the amino acid sequence of SEQ ID NO: 441, and the VL-CDR3 comprises the amino acid sequence of SEQ ID NO: 442;
(v) the VH-CDR1 comprises the amino acid sequence of SEQ ID NO: 443, the VH-CDR2 comprises the amino acid sequence of SEQ ID NO: 444, the VH-CDR3 comprises the amino acid sequence of SEQ ID NO: 445, the VL-CDR1 comprises the amino acid sequence of SEQ ID NO: 446, the VL-CDR2 comprises the amino acid sequence of SEQ ID NO: 447, and the VL-CDR3 comprises the amino acid sequence of SEQ ID NO: 448; or
(vi) the VH-CDR1 comprises the amino acid sequence of SEQ ID NO: 449, the VH-CDR2 comprises the amino acid sequence of SEQ ID NO: 450, the VH-CDR3 comprises the amino acid sequence of SEQ ID NO: 451, the VL-CDR1 comprises the amino acid sequence of SEQ ID NO: 452, the VL-CDR2 comprises the amino acid sequence of GAS, and the VL-CDR3 comprises the amino acid sequence of SEQ ID NO: 454;
(12) (i) the VH-CDR1 comprises the amino acid sequence of SEQ ID NO: 457, the VH-CDR2 comprises the amino acid sequence of SEQ ID NO: 458, the VH-CDR3 comprises the amino acid sequence of SEQ ID NO: 459, the VL-CDR1 comprises the amino acid sequence of SEQ ID NO: 460, the VL-CDR2 comprises the amino acid sequence of SEQ ID NO: 461, and the VL-CDR3 comprises the amino acid sequence of SEQ ID NO: 462;
(ii) the VH-CDR1 comprises the amino acid sequence of SEQ ID NO: 463, the VH-CDR2 comprises the amino acid sequence of SEQ ID NO: 464, the VH-CDR3 comprises the amino acid sequence of SEQ ID NO: 465, the VL-CDR1 comprises the amino acid sequence of SEQ ID NO: 466, the VL-CDR2 comprises the amino acid sequence of SEQ ID NO: 467, and the VL-CDR3 comprises the amino acid sequence of SEQ ID NO: 468;
(iii) the VH-CDR1 comprises the amino acid sequence of SEQ ID NO: 469, the VH-CDR2 comprises the amino acid sequence of SEQ ID NO: 470, the VH-CDR3 comprises the amino acid sequence of SEQ ID NO: 471, the VL-CDR1 comprises the amino acid sequence of SEQ ID NO: 472, the VL-CDR2 comprises the amino acid sequence of GAS, and the VL-CDR3 comprises the amino acid sequence of SEQ ID NO: 474;
(iv) the VH-CDR1 comprises the amino acid sequence of SEQ ID NO: 475, the VH-CDR2 comprises the amino acid sequence of SEQ ID NO: 476, the VH-CDR3 comprises the amino acid sequence of SEQ ID NO: 477, the VL-CDR1 comprises the amino acid sequence of SEQ ID NO: 478, the VL-CDR2 comprises the amino acid sequence of SEQ ID NO: 479, and the VL-CDR3 comprises the amino acid sequence of SEQ ID NO: 480;
(v) the VH-CDR1 comprises the amino acid sequence of SEQ ID NO: 481, the VH-CDR2 comprises the amino acid sequence of SEQ ID NO: 482, the VH-CDR3 comprises the amino acid sequence of SEQ ID NO: 483, the VL-CDR1 comprises the amino acid sequence of SEQ ID NO: 484, the VL-CDR2 comprises the amino acid sequence of SEQ ID NO: 485, and the VL-CDR3 comprises the amino acid sequence of SEQ ID NO: 486; or
(vi) the VH-CDR1 comprises the amino acid sequence of SEQ ID NO: 487, the VH-CDR2 comprises the amino acid sequence of SEQ ID NO: 488, the VH-CDR3 comprises the amino acid sequence of SEQ ID NO: 489, the VL-CDR1 comprises the amino acid sequence of SEQ ID NO: 490, the VL-CDR2 comprises the amino acid sequence of GAS, and the VL-CDR3 comprises the amino acid sequence of SEQ ID NO: 492;
(13) (i) the VH-CDR1 comprises the amino acid sequence of SEQ ID NO: 495, the VH-CDR2 comprises the amino acid sequence of SEQ ID NO: 496, the VH-CDR3 comprises the amino acid sequence of SEQ ID NO: 497, the VL-CDR1 comprises the amino acid sequence of SEQ ID NO: 498, the VL-CDR2 comprises the amino acid sequence of SEQ ID NO: 499, and the VL-CDR3 comprises the amino acid sequence of SEQ ID NO: 500;
(ii) the VH-CDR1 comprises the amino acid sequence of SEQ ID NO: 501, the VH-CDR2 comprises the amino acid sequence of SEQ ID NO: 502, the VH-CDR3 comprises the amino acid sequence of SEQ ID NO: 503, the VL-CDR1 comprises the amino acid sequence of SEQ ID NO: 504, the VL-CDR2 comprises the amino acid sequence of SEQ ID NO: 505, and the VL-CDR3 comprises the amino acid sequence of SEQ ID NO: 506;
(iii) the VH-CDR1 comprises the amino acid sequence of SEQ ID NO: 507, the VH-CDR2 comprises the amino acid sequence of SEQ ID NO: 508, the VH-CDR3 comprises the amino acid sequence of SEQ ID NO: 509, the VL-CDR1 comprises the amino acid sequence of SEQ ID NO: 510, the VL-CDR2 comprises the amino acid sequence of GAD, and the VL-CDR3 comprises the amino acid sequence of SEQ ID NO: 512;
(iv) the VH-CDR1 comprises the amino acid sequence of SEQ ID NO: 513, the VH-CDR2 comprises the amino acid sequence of SEQ ID NO: 514, the VH-CDR3 comprises the amino acid sequence of SEQ ID NO: 515, the VL-CDR1 comprises the amino acid sequence of SEQ ID NO: 516, the VL-CDR2 comprises the amino acid sequence of SEQ ID NO: 517, and the VL-CDR3 comprises the amino acid sequence of SEQ ID NO: 518;
(v) the VH-CDR1 comprises the amino acid sequence of SEQ ID NO: 519, the VH-CDR2 comprises the amino acid sequence of SEQ ID NO: 520, the VH-CDR3 comprises the amino acid sequence of SEQ ID NO: 521, the VL-CDR1 comprises the amino acid sequence of SEQ ID NO: 522, the VL-CDR2 comprises the amino acid sequence of SEQ ID NO: 523, and the VL-CDR3 comprises the amino acid sequence of SEQ ID NO: 524; or
(vi) the VH-CDR1 comprises the amino acid sequence of SEQ ID NO: 525, the VH-CDR2 comprises the amino acid sequence of SEQ ID NO: 526, the VH-CDR3 comprises the amino acid sequence of SEQ ID NO: 527, the VL-CDR1 comprises the amino acid sequence of SEQ ID NO: 528, the VL-CDR2 comprises the amino acid sequence of GAD, and the VL-CDR3 comprises the amino acid sequence of SEQ ID NO: 530;
(14) (i) the VH-CDR1 comprises the amino acid sequence of SEQ ID NO: 533, the VH-CDR2 comprises the amino acid sequence of SEQ ID NO: 534, the VH-CDR3 comprises the amino acid sequence of SEQ ID NO: 535, the VL-CDR1 comprises the amino acid sequence of SEQ ID NO: 536, the VL-CDR2 comprises the amino acid sequence of SEQ ID NO: 537, and the VL-CDR3 comprises the amino acid sequence of SEQ ID NO: 538;
(ii) the VH-CDR1 comprises the amino acid sequence of SEQ ID NO: 539, the VH-CDR2 comprises the amino acid sequence of SEQ ID NO: 540, the VH-CDR3 comprises the amino acid sequence of SEQ ID NO: 541, the VL-CDR1 comprises the amino acid sequence of SEQ ID NO: 542, the VL-CDR2 comprises the amino acid sequence of SEQ ID NO: 543, and the VL-CDR3 comprises the amino acid sequence of SEQ ID NO: 544;
(iii) the VH-CDR1 comprises the amino acid sequence of SEQ ID NO: 545, the VH-CDR2 comprises the amino acid sequence of SEQ ID NO: 546, the VH-CDR3 comprises the amino acid sequence of SEQ ID NO: 547, the VL-CDR1 comprises the amino acid sequence of SEQ ID NO: 548, the VL-CDR2 comprises the amino acid sequence of GAS, and the VL-CDR3 comprises the amino acid sequence of SEQ ID NO: 550;
(iv) the VH-CDR1 comprises the amino acid sequence of SEQ ID NO: 551, the VH-CDR2 comprises the amino acid sequence of SEQ ID NO: 552, the VH-CDR3 comprises the amino acid sequence of SEQ ID NO: 553, the VL-CDR1 comprises the amino acid sequence of SEQ ID NO: 554, the VL-CDR2 comprises the amino acid sequence of SEQ ID NO: 555, and the VL-CDR3 comprises the amino acid sequence of SEQ ID NO: 556;
(v) the VH-CDR1 comprises the amino acid sequence of SEQ ID NO: 557, the VH-CDR2 comprises the amino acid sequence of SEQ ID NO: 558, the VH-CDR3 comprises the amino acid sequence of SEQ ID NO: 559, the VL-CDR1 comprises the amino acid sequence of SEQ ID NO: 560, the VL-CDR2 comprises the amino acid sequence of SEQ ID NO: 561, and the VL-CDR3 comprises the amino acid sequence of SEQ ID NO: 562; or
(vi) the VH-CDR1 comprises the amino acid sequence of SEQ ID NO: 563, the VH-CDR2 comprises the amino acid sequence of SEQ ID NO: 564, the VH-CDR3 comprises the amino acid sequence of SEQ ID NO: 565, the VL-CDR1 comprises the amino acid sequence of SEQ ID NO: 566, the VL-CDR2 comprises the amino acid sequence of GAS, and the VL-CDR3 comprises the amino acid sequence of SEQ ID NO: 568;
(15) (i) the VH-CDR1 comprises the amino acid sequence of SEQ ID NO: 571, the VH-CDR2 comprises the amino acid sequence of SEQ ID NO: 572, the VH-CDR3 comprises the amino acid sequence of SEQ ID NO: 573, the VL-CDR1 comprises the amino acid sequence of SEQ ID NO: 574, the VL-CDR2 comprises the amino acid sequence of SEQ ID NO: 575, and the VL-CDR3 comprises the amino acid sequence of SEQ ID NO: 576;
(ii) the VH-CDR1 comprises the amino acid sequence of SEQ ID NO: 577, the VH-CDR2 comprises the amino acid sequence of SEQ ID NO: 578, the VH-CDR3 comprises the amino acid sequence of SEQ ID NO: 579, the VL-CDR1 comprises the amino acid sequence of SEQ ID NO: 580, the VL-CDR2 comprises the amino acid sequence of SEQ ID NO: 581, and the VL-CDR3 comprises the amino acid sequence of SEQ ID NO: 582;
(iii) the VH-CDR1 comprises the amino acid sequence of SEQ ID NO: 583, the VH-CDR2 comprises the amino acid sequence of SEQ ID NO: 584, the VH-CDR3 comprises the amino acid sequence of SEQ ID NO: 585, the VL-CDR1 comprises the amino acid sequence of SEQ ID NO: 586, the VL-CDR2 comprises the amino acid sequence of GAF, and the VL-CDR3 comprises the amino acid sequence of SEQ ID NO: 588;
(iv) the VH-CDR1 comprises the amino acid sequence of SEQ ID NO: 589, the VH-CDR2 comprises the amino acid sequence of SEQ ID NO: 590, the VH-CDR3 comprises the amino acid sequence of SEQ ID NO: 591, the VL-CDR1 comprises the amino acid sequence of SEQ ID NO: 592, the VL-CDR2 comprises the amino acid sequence of SEQ ID NO: 593, and the VL-CDR3 comprises the amino acid sequence of SEQ ID NO: 594;
(v) the VH-CDR1 comprises the amino acid sequence of SEQ ID NO: 595, the VH-CDR2 comprises the amino acid sequence of SEQ ID NO: 596, the VH-CDR3 comprises the amino acid sequence of SEQ ID NO: 597, the VL-CDR1 comprises the amino acid sequence of SEQ ID NO: 598, the VL-CDR2 comprises the amino acid sequence of SEQ ID NO: 599, and the VL-CDR3 comprises the amino acid sequence of SEQ ID NO: 600; or
(vi) the VH-CDR1 comprises the amino acid sequence of SEQ ID NO: 601, the VH-CDR2 comprises the amino acid sequence of SEQ ID NO: 602, the VH-CDR3 comprises the amino acid sequence of SEQ ID NO: 603, the VL-CDR1 comprises the amino acid sequence of SEQ ID NO: 604, the VL-CDR2 comprises the amino acid sequence of GAF, and the VL-CDR3 comprises the amino acid sequence of SEQ ID NO: 606;
(16) (i) the VH-CDR1 comprises the amino acid sequence of SEQ ID NO: 609, the VH-CDR2 comprises the amino acid sequence of SEQ ID NO: 610, the VH-CDR3 comprises the amino acid sequence of SEQ ID NO: 611, the VL-CDR1 comprises the amino acid sequence of SEQ ID NO: 612, the VL-CDR2 comprises the amino acid sequence of SEQ ID NO: 613, and the VL-CDR3 comprises the amino acid sequence of SEQ ID NO: 614;
(ii) the VH-CDR1 comprises the amino acid sequence of SEQ ID NO: 615, the VH-CDR2 comprises the amino acid sequence of SEQ ID NO: 616, the VH-CDR3 comprises the amino acid sequence of SEQ ID NO: 617, the VL-CDR1 comprises the amino acid sequence of SEQ ID NO: 618, the VL-CDR2 comprises the amino acid sequence of SEQ ID NO: 619, and the VL-CDR3 comprises the amino acid sequence of SEQ ID NO: 620;
(iii) the VH-CDR1 comprises the amino acid sequence of SEQ ID NO: 621, the VH-CDR2 comprises the amino acid sequence of SEQ ID NO: 622, the VH-CDR3 comprises the amino acid sequence of SEQ ID NO: 623, the VL-CDR1 comprises the amino acid sequence of SEQ ID NO: 624, the VL-CDR2 comprises the amino acid sequence of GAY, and the VL-CDR3 comprises the amino acid sequence of SEQ ID NO: 626;
(iv) the VH-CDR1 comprises the amino acid sequence of SEQ ID NO: 627, the VH-CDR2 comprises the amino acid sequence of SEQ ID NO: 628, the VH-CDR3 comprises the amino acid sequence of SEQ ID NO: 629, the VL-CDR1 comprises the amino acid sequence of SEQ ID NO: 630, the VL-CDR2 comprises the amino acid sequence of SEQ ID NO: 631, and the VL-CDR3 comprises the amino acid sequence of SEQ ID NO: 632;
(v) the VH-CDR1 comprises the amino acid sequence of SEQ ID NO: 633, the VH-CDR2 comprises the amino acid sequence of SEQ ID NO: 634, the VH-CDR3 comprises the amino acid sequence of SEQ ID NO: 635, the VL-CDR1 comprises the amino acid sequence of SEQ ID NO: 636, the VL-CDR2 comprises the amino acid sequence of SEQ ID NO: 637, and the VL-CDR3 comprises the amino acid sequence of SEQ ID NO: 638; or
(vi) the VH-CDR1 comprises the amino acid sequence of SEQ ID NO: 639, the VH-CDR2 comprises the amino acid sequence of SEQ ID NO: 640, the VH-CDR3 comprises the amino acid sequence of SEQ ID NO: 641, the VL-CDR1 comprises the amino acid sequence of SEQ ID NO: 642, the VL-CDR2 comprises the amino acid sequence of GAY, and the VL-CDR3 comprises the amino acid sequence of SEQ ID NO: 644;
(17) (i) the VH-CDR1 comprises the amino acid sequence of SEQ ID NO: 647, the VH-CDR2 comprises the amino acid sequence of SEQ ID NO: 648, the VH-CDR3 comprises the amino acid sequence of SEQ ID NO: 649, the VL-CDR1 comprises the amino acid sequence of SEQ ID NO: 650, the VL-CDR2 comprises the amino acid sequence of SEQ ID NO: 651, and the VL-CDR3 comprises the amino acid sequence of SEQ ID NO: 652;
(ii) the VH-CDR1 comprises the amino acid sequence of SEQ ID NO: 653, the VH-CDR2 comprises the amino acid sequence of SEQ ID NO: 654, the VH-CDR3 comprises the amino acid sequence of SEQ ID NO: 655, the VL-CDR1 comprises the amino acid sequence of SEQ ID NO: 656, the VL-CDR2 comprises the amino acid sequence of SEQ ID NO: 657, and the VL-CDR3 comprises the amino acid sequence of SEQ ID NO: 658;
(iii) the VH-CDR1 comprises the amino acid sequence of SEQ ID NO: 659, the VH-CDR2 comprises the amino acid sequence of SEQ ID NO: 660, the VH-CDR3 comprises the amino acid sequence of SEQ ID NO: 661, the VL-CDR1 comprises the amino acid sequence of SEQ ID NO: 662, the VL-CDR2 comprises the amino acid sequence of GAS, and the VL-CDR3 comprises the amino acid sequence of SEQ ID NO: 664;
(iv) the VH-CDR1 comprises the amino acid sequence of SEQ ID NO: 665, the VH-CDR2 comprises the amino acid sequence of SEQ ID NO: 666, the VH-CDR3 comprises the amino acid sequence of SEQ ID NO: 667, the VL-CDR1 comprises the amino acid sequence of SEQ ID NO: 668, the VL-CDR2 comprises the amino acid sequence of SEQ ID NO: 669, and the VL-CDR3 comprises the amino acid sequence of SEQ ID NO: 670;
(v) the VH-CDR1 comprises the amino acid sequence of SEQ ID NO: 671, the VH-CDR2 comprises the amino acid sequence of SEQ ID NO: 672, the VH-CDR3 comprises the amino acid sequence of SEQ ID NO: 673, the VL-CDR1 comprises the amino acid sequence of SEQ ID NO: 674, the VL-CDR2 comprises the amino acid sequence of SEQ ID NO: 675, and the VL-CDR3 comprises the amino acid sequence of SEQ ID NO: 676; or
(vi) the VH-CDR1 comprises the amino acid sequence of SEQ ID NO: 677, the VH-CDR2 comprises the amino acid sequence of SEQ ID NO: 678, the VH-CDR3 comprises the amino acid sequence of SEQ ID NO: 679, the VL-CDR1 comprises the amino acid sequence of SEQ ID NO: 680, the VL-CDR2 comprises the amino acid sequence of GAS, and the VL-CDR3 comprises the amino acid sequence of SEQ ID NO: 682;
(18) (i) the VH-CDR1 comprises the amino acid sequence of SEQ ID NO: 685, the VH-CDR2 comprises the amino acid sequence of SEQ ID NO: 686, the VH-CDR3 comprises the amino acid sequence of SEQ ID NO: 687, the VL-CDR1 comprises the amino acid sequence of SEQ ID NO: 688, the VL-CDR2 comprises the amino acid sequence of SEQ ID NO: 689, and the VL-CDR3 comprises the amino acid sequence of SEQ ID NO: 690;
(ii) the VH-CDR1 comprises the amino acid sequence of SEQ ID NO: 691, the VH-CDR2 comprises the amino acid sequence of SEQ ID NO: 692, the VH-CDR3 comprises the amino acid sequence of SEQ ID NO: 693, the VL-CDR1 comprises the amino acid sequence of SEQ ID NO: 694, the VL-CDR2 comprises the amino acid sequence of SEQ ID NO: 695, and the VL-CDR3 comprises the amino acid sequence of SEQ ID NO: 696;
(iii) the VH-CDR1 comprises the amino acid sequence of SEQ ID NO: 697, the VH-CDR2 comprises the amino acid sequence of SEQ ID NO: 698, the VH-CDR3 comprises the amino acid sequence of SEQ ID NO: 699, the VL-CDR1 comprises the amino acid sequence of SEQ ID NO: 700, the VL-CDR2 comprises the amino acid sequence of GAS, and the VL-CDR3 comprises the amino acid sequence of SEQ ID NO: 702;
(iv) the VH-CDR1 comprises the amino acid sequence of SEQ ID NO: 703, the VH-CDR2 comprises the amino acid sequence of SEQ ID NO: 704, the VH-CDR3 comprises the amino acid sequence of SEQ ID NO: 705, the VL-CDR1 comprises the amino acid sequence of SEQ ID NO: 706, the VL-CDR2 comprises the amino acid sequence of SEQ ID NO: 707, and the VL-CDR3 comprises the amino acid sequence of SEQ ID NO: 708;
(v) the VH-CDR1 comprises the amino acid sequence of SEQ ID NO: 709, the VH-CDR2 comprises the amino acid sequence of SEQ ID NO: 710, the VH-CDR3 comprises the amino acid sequence of SEQ ID NO: 711, the VL-CDR1 comprises the amino acid sequence of SEQ ID NO: 712, the VL-CDR2 comprises the amino acid sequence of SEQ ID NO: 713, and the VL-CDR3 comprises the amino acid sequence of SEQ ID NO: 714; or
(vi) the VH-CDR1 comprises the amino acid sequence of SEQ ID NO: 715, the VH-CDR2 comprises the amino acid sequence of SEQ ID NO: 716, the VH-CDR3 comprises the amino acid sequence of SEQ ID NO: 717, the VL-CDR1 comprises the amino acid sequence of SEQ ID NO: 718, the VL-CDR2 comprises the amino acid sequence of GAS, and the VL-CDR3 comprises the amino acid sequence of SEQ ID NO: 720; or
(19) (i) the VH-CDR1 comprises the amino acid sequence of SEQ ID NO: 723, the VH-CDR2 comprises the amino acid sequence of SEQ ID NO: 724, the VH-CDR3 comprises the amino acid sequence of SEQ ID NO: 725, the VL-CDR1 comprises the amino acid sequence of SEQ ID NO: 726, the VL-CDR2 comprises the amino acid sequence of SEQ ID NO: 727, and the VL-CDR3 comprises the amino acid sequence of SEQ ID NO: 728;
(ii) the VH-CDR1 comprises the amino acid sequence of SEQ ID NO: 729, the VH-CDR2 comprises the amino acid sequence of SEQ ID NO: 730, the VH-CDR3 comprises the amino acid sequence of SEQ ID NO: 731, the VL-CDR1 comprises the amino acid sequence of SEQ ID NO: 732, the VL-CDR2 comprises the amino acid sequence of SEQ ID NO: 733, and the VL-CDR3 comprises the amino acid sequence of SEQ ID NO: 734;
(iii) the VH-CDR1 comprises the amino acid sequence of SEQ ID NO: 735, the VH-CDR2 comprises the amino acid sequence of SEQ ID NO: 736, the VH-CDR3 comprises the amino acid sequence of SEQ ID NO: 737, the VL-CDR1 comprises the amino acid sequence of SEQ ID NO: 738, the VL-CDR2 comprises the amino acid sequence of DAS, and the VL-CDR3 comprises the amino acid sequence of SEQ ID NO: 740;
(iv) the VH-CDR1 comprises the amino acid sequence of SEQ ID NO: 741, the VH-CDR2 comprises the amino acid sequence of SEQ ID NO: 742, the VH-CDR3 comprises the amino acid sequence of SEQ ID NO: 743, the VL-CDR1 comprises the amino acid sequence of SEQ ID NO: 744, the VL-CDR2 comprises the amino acid sequence of SEQ ID NO: 745, and the VL-CDR3 comprises the amino acid sequence of SEQ ID NO: 746;
(v) the VH-CDR1 comprises the amino acid sequence of SEQ ID NO: 747, the VH-CDR2 comprises the amino acid sequence of SEQ ID NO: 748, the VH-CDR3 comprises the amino acid sequence of SEQ ID NO: 749, the VL-CDR1 comprises the amino acid sequence of SEQ ID NO: 750, the VL-CDR2 comprises the amino acid sequence of SEQ ID NO: 751, and the VL-CDR3 comprises the amino acid sequence of SEQ ID NO: 752; or
(vi) the VH-CDR1 comprises the amino acid sequence of SEQ ID NO: 753, the VH-CDR2 comprises the amino acid sequence of SEQ ID NO: 754, the VH-CDR3 comprises the amino acid sequence of SEQ ID NO: 755, the VL-CDR1 comprises the amino acid sequence of SEQ ID NO: 756, the VL-CDR2 comprises the amino acid sequence of DAS, and the VL-CDR3 comprises the amino acid sequence of SEQ ID NO: 758.

507. The antibody of claim 504, wherein the antibody comprises:
(1) a VH sequence of SEQ ID NO: 37 and a VL sequence of SEQ ID NO: 38;
(2) a VH sequence of SEQ ID NO: 75 and a VL sequence of SEQ ID NO: 76;
(3) a VH sequence of SEQ ID NO: 113 and a VL sequence of SEQ ID NO: 114;
(4) a VH sequence of SEQ ID NO: 189 and a VL sequence of SEQ ID NO: 190;
(5) a VH sequence of SEQ ID NO: 227 and a VL sequence of SEQ ID NO: 228;
(6) a VH sequence of SEQ ID NO: 265 and a VL sequence of SEQ ID NO: 266;
(7) a VH sequence of SEQ ID NO: 303 and a VL sequence of SEQ ID NO: 304;
(8) a VH sequence of SEQ ID NO: 341 and a VL sequence of SEQ ID NO: 342;
(9) a VH sequence of SEQ ID NO: 379 and a VL sequence of SEQ ID NO: 380;
(10) a VH sequence of SEQ ID NO: 417 and a VL sequence of SEQ ID NO: 418;
(11) a VH sequence of SEQ ID NO: 455 and a VL sequence of SEQ ID NO: 456;
(12) a VH sequence of SEQ ID NO: 493 and a VL sequence of SEQ ID NO: 494;
(13) a VH sequence of SEQ ID NO: 531 and a VL sequence of SEQ ID NO: 532;
(14) a VH sequence of SEQ ID NO: 569 and a VL sequence of SEQ ID NO: 570;
(15) a VH sequence of SEQ ID NO: 607 and a VL sequence of SEQ ID NO: 608;
(16) a VH sequence of SEQ ID NO: 645 and a VL sequence of SEQ ID NO: 646;
(17) a VH sequence of SEQ ID NO: 683 and a VL sequence of SEQ ID NO: 684;
(18) a VH sequence of SEQ ID NO: 721 and a VL sequence of SEQ ID NO: 722; or
(19) a VH sequence of SEQ ID NO: 759 and a VL sequence of SEQ ID NO: 760.

508. The antibody of claim 504, wherein the antibody is human, humanized, or chimeric.

509. The antibody of claim 504, wherein the antibody is a monoclonal antibody.

510. The antibody of claim 504, wherein the antibody is a Fab, Fab′, F(ab′)2, Fv, scFv, (scFv)2, single chain antibody molecule, dual variable domain antibody, linear antibody, or V domain antibody.

511. The antibody of claim 504, wherein the antibody comprises an Fc region.

512. The antibody of claim 504, wherein the antibody comprises a heavy chain constant region of a class selected from IgG, IgA, IgD, IgE, and IgM.

513. The antibody of claim 504, wherein the antibody comprises a heavy chain constant region of the class IgG and a subclass selected from IgG1, IgG2, IgG3, and IgG4.

514. An isolated polynucleotide or set of polynucleotides encoding the antibody of claim 504, a VH thereof, a VL thereof, a light chain thereof, a heavy chain thereof, or an antigen-binding portion thereof.

515. A vector or set of vectors comprising the polynucleotide or set of polynucleotides of claim 514.

516. A host cell comprising the polynucleotide or set of polynucleotides of claim 514.

517. A method of producing an antibody comprising expressing the antibody with the host cell of claim 516 and isolating the expressed antibody.

518. A pharmaceutical composition comprising the antibody of claim 504 and a pharmaceutically acceptable excipient.

519. A kit comprising
(i) the antibody of claim 504; and
(ii) instructions for use.


SEQUENCE LISTING (922 sequences)

SEQ ID NO: 1 - protein, 10 aa, synthetic construct
GFTFSDYAMG

SEQ ID NO: 2 - protein, 17 aa, synthetic construct
TISGSGGLTYYADSVKG

SEQ ID NO: 3 - protein, 9 aa, synthetic construct
APYGYYMDV

SEQ ID NO: 4 - protein, 11 aa, synthetic construct
RASQSISSWLA

SEQ ID NO: 5 - protein, 7 aa, synthetic construct
KASSLES

SEQ ID NO: 6 - protein, 8 aa, synthetic construct
QQYKSYIT

SEQ ID NO: 7 - protein, 5 aa, synthetic construct
DYAMG

SEQ ID NO: 8 - protein, 17 aa, synthetic construct
TISGSGGLTYYADSVKG

SEQ ID NO: 9 - protein, 9 aa, synthetic construct
APYGYYMDV

SEQ ID NO: 10 - protein, 11 aa, synthetic construct
RASQSISSWLA

SEQ ID NO: 11 - protein, 7 aa, synthetic construct
KASSLES

SEQ ID NO: 12 - protein, 8 aa, synthetic construct
QQYKSYIT

SEQ ID NO: 13 - protein, 7 aa, synthetic construct
GFTFSDY

SEQ ID NO: 14 - protein, 4 aa, synthetic construct
GSGG

SEQ ID NO: 15 - protein, 7 aa, synthetic construct
PYGYYMD

SEQ ID NO: 16 - protein, 7 aa, synthetic construct
SQSISSW

SEQ ID NO: 17 - skipped

SEQ ID NO: 18 - protein, 5 aa, synthetic construct
YKSYI

SEQ ID NO: 19 - protein, 10 aa, synthetic construct
GFTFSDYAMG

SEQ ID NO: 20 - protein, 10 aa, synthetic construct
TISGSGGLTY

SEQ ID NO: 21 - protein, 9 aa, synthetic construct
APYGYYMDV

SEQ ID NO: 22 - protein, 11 aa, synthetic construct
RASQSISSWLA

SEQ ID NO: 23 - protein, 7 aa, synthetic construct
KASSLES

SEQ ID NO: 24 - protein, 8 aa, synthetic construct
QQYKSYIT

SEQ ID NO: 25 - protein, 6 aa, synthetic construct
SDYAMG

SEQ ID NO: 26 - protein, 13 aa, synthetic construct
WVSTISGSGGLTY

SEQ ID NO: 27 - protein, 10 aa, synthetic construct
AKAPYGYYMD

SEQ ID NO: 28 - protein, 7 aa, synthetic construct
SSWLAWY

SEQ ID NO: 29 - protein, 10 aa, synthetic construct
LLIYKASSLE

SEQ ID NO: 30 - protein, 7 aa, synthetic construct
QQYKSYI

SEQ ID NO: 31 - protein, 8 aa, synthetic construct
GFTFSDYA

SEQ ID NO: 32 - protein, 8 aa, synthetic construct
ISGSGGLT

SEQ ID NO: 33 - protein, 11 aa, synthetic construct
AKAPYGYYMDV

SEQ ID NO: 34 - protein, 6 aa, synthetic construct
QSISSW

SEQ ID NO: 35 - skipped

SEQ ID NO: 36 - protein, 8 aa, synthetic construct
QQYKSYIT

SEQ ID NO: 37 - protein, 118 aa, synthetic construct
EVQLLESGGGLVQPGGSLRLSCAASGFTFSDYAMGWVRQAPGKGLEWVSTISGSGGLTYYADSVKGRFTISRDNSKNTLYLQMNSLRAEDTAVYYCAKAPYGYYMDVWGKGTTVTVSS

SEQ ID NO: 38 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYKSYITFGGGTKVEIK

SEQ ID NO: 39 - protein, 10 aa, synthetic construct
GFTFSSYAMA

SEQ ID NO: 40 - protein, 17 aa, synthetic construct
AISGSGGLTYYADSVKG

SEQ ID NO: 41 - protein, 9 aa, synthetic construct
APYGYYMDV

SEQ ID NO: 42 - protein, 11 aa, synthetic construct
RASQSISSWLA

SEQ ID NO: 43 - protein, 7 aa, synthetic construct
KASSLES

SEQ ID NO: 44 - protein, 8 aa, synthetic construct
QQYKSYIT

SEQ ID NO: 45 - protein, 5 aa, synthetic construct
SYAMA

SEQ ID NO: 46 - protein, 17 aa, synthetic construct
AISGSGGLTYYADSVKG

SEQ ID NO: 47 - protein, 9 aa, synthetic construct
APYGYYMDV

SEQ ID NO: 48 - protein, 11 aa, synthetic construct
RASQSISSWLA

SEQ ID NO: 49 - protein, 7 aa, synthetic construct
KASSLES

SEQ ID NO: 50 - protein, 8 aa, synthetic construct
QQYKSYIT

SEQ ID NO: 51 - protein, 7 aa, synthetic construct
GFTFSSY

SEQ ID NO: 52 - protein, 4 aa, synthetic construct
GSGG

SEQ ID NO: 53 - protein, 7 aa, synthetic construct
PYGYYMD

SEQ ID NO: 54 - protein, 7 aa, synthetic construct
SQSISSW

SEQ ID NO: 55 - skipped

SEQ ID NO: 56 - protein, 5 aa, synthetic construct
YKSYI

SEQ ID NO: 57 - protein, 10 aa, synthetic construct
GFTFSSYAMA

SEQ ID NO: 58 - protein, 10 aa, synthetic construct
AISGSGGLTY

SEQ ID NO: 59 - protein, 9 aa, synthetic construct
APYGYYMDV

SEQ ID NO: 60 - protein, 11 aa, synthetic construct
RASQSISSWLA

SEQ ID NO: 61 - protein, 7 aa, synthetic construct
KASSLES

SEQ ID NO: 62 - protein, 8 aa, synthetic construct
QQYKSYIT

SEQ ID NO: 63 - protein, 6 aa, synthetic construct
SSYAMA

SEQ ID NO: 64 - protein, 13 aa, synthetic construct
WVSAISGSGGLTY

SEQ ID NO: 65 - protein, 10 aa, synthetic construct
AKAPYGYYMD

SEQ ID NO: 66 - protein, 7 aa, synthetic construct
SSWLAWY

SEQ ID NO: 67 - protein, 10 aa, synthetic construct
LLIYKASSLE

SEQ ID NO: 68 - protein, 7 aa, synthetic construct
QQYKSYI

SEQ ID NO: 69 - protein, 8 aa, synthetic construct
GFTFSSYA

SEQ ID NO: 70 - protein, 8 aa, synthetic construct
ISGSGGLT

SEQ ID NO: 71 - protein, 11 aa, synthetic construct
AKAPYGYYMDV

SEQ ID NO: 72 - protein, 6 aa, synthetic construct
QSISSW

SEQ ID NO: 73 - skipped

SEQ ID NO: 74 - protein, 8 aa, synthetic construct
QQYKSYIT

SEQ ID NO: 75 - protein, 118 aa, synthetic construct
EVQLLESGGGLVQPGGSLRLSCAASGFTFSSYAMAWVRQAPGKGLEWVSAISGSGGLTYYADSVKGRFTISRDNSKNTLYLQMNSLRAEDTAVYYCAKAPYGYYMDVWGKGTTVTVSS

SEQ ID NO: 76 - protein, 106 aa, synthetic construct; identical to SEQ ID NO: 38

SEQ ID NO: 77 - protein, 10 aa, synthetic construct
GYTFDVYGIS

SEQ ID NO: 78 - protein, 17 aa, synthetic construct
WIAPYNGNTNYAQKLQG

SEQ ID NO: 79 - protein, 14 aa, synthetic construct
DAGTYSPFGYGMDV

SEQ ID NO: 80 - protein, 11 aa, synthetic construct
RASQSISSWLA

SEQ ID NO: 81 - protein, 7 aa, synthetic construct
KASSLES

SEQ ID NO: 82 - protein, 10 aa, synthetic construct
QQFQSLPPFT

SEQ ID NO: 83 - protein, 5 aa, synthetic construct
VYGIS

SEQ ID NO: 84 - protein, 17 aa, synthetic construct
WIAPYNGNTNYAQKLQG

SEQ ID NO: 85 - protein, 14 aa, synthetic construct
DAGTYSPFGYGMDV

SEQ ID NO: 86 - protein, 11 aa, synthetic construct
RASQSISSWLA

SEQ ID NO: 87 - protein, 7 aa, synthetic construct
KASSLES

SEQ ID NO: 88 - protein, 10 aa, synthetic construct
QQFQSLPPFT

SEQ ID NO: 89 - protein, 7 aa, synthetic construct
GYTFDVY

SEQ ID NO: 90 - protein, 4 aa, synthetic construct
PYNG

SEQ ID NO: 91 - protein, 12 aa, synthetic construct
AGTYSPFGYGMD

SEQ ID NO: 92 - protein, 7 aa, synthetic construct
SQSISSW

SEQ ID NO: 93 - skipped

SEQ ID NO: 94 - protein, 7 aa, synthetic construct
FQSLPPF

SEQ ID NO: 95 - protein, 10 aa, synthetic construct
GYTFDVYGIS

SEQ ID NO: 96 - protein, 10 aa, synthetic construct
WIAPYNGNTN

SEQ ID NO: 97 - protein, 14 aa, synthetic construct
DAGTYSPFGYGMDV

SEQ ID NO: 98 - protein, 11 aa, synthetic construct
RASQSISSWLA

SEQ ID NO: 99 - protein, 7 aa, synthetic construct
KASSLES

SEQ ID NO: 100 - protein, 10 aa, synthetic construct
QQFQSLPPFT

SEQ ID NO: 101 - protein, 6 aa, synthetic construct
DVYGIS

SEQ ID NO: 102 - protein, 13 aa, synthetic construct
WMGWIAPYNGNTN

SEQ ID NO: 103 - protein, 15 aa, synthetic construct
ARDAGTYSPFGYGMD

SEQ ID NO: 104 - protein, 7 aa, synthetic construct
SSWLAWY

SEQ ID NO: 105 - protein, 10 aa, synthetic construct
LLIYKASSLE

SEQ ID NO: 106 - protein, 9 aa, synthetic construct
QQFQSLPPF

SEQ ID NO: 107 - protein, 8 aa, synthetic construct
GYTFDVYG

SEQ ID NO: 108 - protein, 8 aa, synthetic construct
IAPYNGNT

SEQ ID NO: 109 - protein, 16 aa, synthetic construct
ARDAGTYSPFGTGMDV

SEQ ID NO: 110 - protein, 6 aa, synthetic construct
QSISSW

SEQ ID NO: 111 - skipped

SEQ ID NO: 112 - protein, 10 aa, synthetic construct
QQFQSLPPFT

SEQ ID NO: 113 - protein, 123 aa, synthetic construct
QVQLVQSGAEVKKPGASVKVSCKASGYTFDVYGISWVRQAPGQGLEWMGWIAPYNGNTNYAQKLQGRVTMTTDTSTSTAYMELRSLRSDDTAVYYCARDAGTYSPFGYGMDVWGQGTTVTVSS

SEQ ID NO: 114 - protein, 108 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQFQSLPPFTFGGGTKVEIK

SEQ ID NO: 115 - protein, 10 aa, synthetic construct
GYTFDVYGIS

SEQ ID NO: 116 - protein, 17 aa, synthetic construct
WIAPYSGNTNYAQKLQG

SEQ ID NO: 117 - protein, 14 aa, synthetic construct
DAGTYSPFGYGMDV

SEQ ID NO: 118 - protein, 11 aa, synthetic construct
QASQSINNWLA

SEQ ID NO: 119 - protein, 7 aa, synthetic construct
KAYNLES

SEQ ID NO: 120 - protein, 10 aa, synthetic construct
QLFQSLPPFT

SEQ ID NO: 121 - protein, 5 aa, synthetic construct
VYGIS

SEQ ID NO: 122 - protein, 17 aa, synthetic construct
WIAPYSGNTNYAQKLQG

SEQ ID NO: 123 - protein, 14 aa, synthetic construct
DAGTYSPFGYGMDV

SEQ ID NO: 124 - protein, 11 aa, synthetic construct
QASQSINNWLA

SEQ ID NO: 125 - protein, 7 aa, synthetic construct
KAYNLES

SEQ ID NO: 126 - protein, 10 aa, synthetic construct
QLFQSLPPFT

SEQ ID NO: 127 - protein, 7 aa, synthetic construct
GYTFDVY

SEQ ID NO: 128 - protein, 4 aa, synthetic construct
PYSG

SEQ ID NO: 129 - protein, 12 aa, synthetic construct
AGTYSPFGYGMD

SEQ ID NO: 130 - protein, 7 aa, synthetic construct
SQSINNW

SEQ ID NO: 131 - skipped

SEQ ID NO: 132 - protein, 7 aa, synthetic construct
FQSLPPF

SEQ ID NO: 133 - protein, 10 aa, synthetic construct
GYTFDVYGIS

SEQ ID NO: 134 - protein, 10 aa, synthetic construct
WIAPYSGNTN

SEQ ID NO: 135 - protein, 14 aa, synthetic construct
DAGTYSPFGYGMDV

SEQ ID NO: 136 - protein, 11 aa, synthetic construct
QASQSINNWLA

SEQ ID NO: 137 - protein, 7 aa, synthetic construct
KAYNLES

SEQ ID NO: 138 - protein, 10 aa, synthetic construct
QLFQSLPPFT

SEQ ID NO: 139 - protein, 6 aa, synthetic construct
DVYGIS

SEQ ID NO: 140 - protein, 13 aa, synthetic construct
WMGWIAPYSGNTN

SEQ ID NO: 141 - protein, 15 aa, synthetic construct
ARDAGTYSPFGYGMD

SEQ ID NO: 142 - protein, 7 aa, synthetic construct
NNWLAWY

SEQ ID NO: 143 - protein, 10 aa, synthetic construct
LLIYKAYNLE

SEQ ID NO: 144 - protein, 9 aa, synthetic construct
QLFQSLPPF

SEQ ID NO: 145 - protein, 8 aa, synthetic construct
GYTFDVYG

SEQ ID NO: 146 - protein, 8 aa, synthetic construct
IAPYSGNT

SEQ ID NO: 147 - protein, 16 aa, synthetic construct
ARDAGTYSPFGTGMDV

SEQ ID NO: 148 - protein, 6 aa, synthetic construct
QSINNW

SEQ ID NO: 149 - skipped

SEQ ID NO: 150 - protein, 10 aa, synthetic construct
QLFQSLPPFT

SEQ ID NO: 151 - protein, 123 aa, synthetic construct
QVQLVQSGAEVKKPGASVKVSCKASGYTFDVYGISWVRQAPGQGLEWMGWIAPYSGNTNYAQKLQGRVTMTTDTSTSTAYMELRSLRSDDTAVYYCARDAGTYSPFGYGMDVWGQGTTVTVSS

SEQ ID NO: 152 - protein, 108 aa, synthetic construct
DIQMTQSPSTLSASVGDRVTITCQASQSINNWLAWYQQKPGKAPKLLIYKAYNLESGVPSRFSGSGSGTEFTLTISSLQPDDFATYYCQLFQSLPPFTFGGGTKVEIK

SEQ ID NO: 153 - protein, 10 aa, synthetic construct
GYTFDVYGIS

SEQ ID NO: 154 - protein, 17 aa, synthetic construct
WIAPYSGNTNYAQKLQG

SEQ ID NO: 155 - protein, 14 aa, synthetic construct
DAGTYSPFGYGMDV

SEQ ID NO: 156 - protein, 11 aa, synthetic construct
RASESISNWLA

SEQ ID NO: 157 - protein, 7 aa, synthetic construct
KAYSLEY

SEQ ID NO: 158 - protein, 10 aa, synthetic construct
QQFQKLPPFT

SEQ ID NO: 159 - protein, 5 aa, synthetic construct
VYGIS

SEQ ID NO: 160 - protein, 17 aa, synthetic construct
WIAPYSGNTNYAQKLQG

SEQ ID NO: 161 - protein, 14 aa, synthetic construct
DAGTYSPFGYGMDV

SEQ ID NO: 162 - protein, 11 aa, synthetic construct
RASESISNWLA

SEQ ID NO: 163 - protein, 7 aa, synthetic construct
KAYSLEY

SEQ ID NO: 164 - protein, 10 aa, synthetic construct
QQFQKLPPFT

SEQ ID NO: 165 - protein, 7 aa, synthetic construct
GYTFDVY

SEQ ID NO: 166 - protein, 4 aa, synthetic construct
PYSG

SEQ ID NO: 167 - protein, 12 aa, synthetic construct
AGTYSPFGYGMD

SEQ ID NO: 168 - protein, 7 aa, synthetic construct
SESISNW

SEQ ID NO: 169 - skipped

SEQ ID NO: 170 - protein, 7 aa, synthetic construct
FQKLPPF

SEQ ID NO: 171 - protein, 10 aa, synthetic construct
GYTFDVYGIS

SEQ ID NO: 172 - protein, 10 aa, synthetic construct
WIAPYSGNTN

SEQ ID NO: 173 - protein, 14 aa, synthetic construct
DAGTYSPFGYGMDV

SEQ ID NO: 174 - protein, 11 aa, synthetic construct
RASESISNWLA

SEQ ID NO: 175 - protein, 7 aa, synthetic construct
KAYSLEY

SEQ ID NO: 176 - protein, 10 aa, synthetic construct
QQFQKLPPFT

SEQ ID NO: 177 - protein, 6 aa, synthetic construct
DVYGIS

SEQ ID NO: 178 - protein, 13 aa, synthetic construct
WMGWIAPYSGNTN

SEQ ID NO: 179 - protein, 15 aa, synthetic construct
ARDAGTYSPFGYGMD

SEQ ID NO: 180 - protein, 7 aa, synthetic construct
SNWLAWY

SEQ ID NO: 181 - protein, 10 aa, synthetic construct
LLIYKAYSLE

SEQ ID NO: 182 - protein, 9 aa, synthetic construct
QQFQKLPPF

SEQ ID NO: 183 - protein, 8 aa, synthetic construct
GYTFDVYG

SEQ ID NO: 184 - protein, 8 aa, synthetic construct
IAPYSGNT

SEQ ID NO: 185 - protein, 16 aa, synthetic construct
ARDAGTYSPFGTGMDV

SEQ ID NO: 186 - protein, 6 aa, synthetic construct
ESISNW

SEQ ID NO: 187 - skipped

SEQ ID NO: 188 - protein, 10 aa, synthetic construct
QQFQKLPPFT

SEQ ID NO: 189 - protein, 123 aa, synthetic construct; identical to SEQ ID NO: 151

SEQ ID NO: 190 - protein, 108 aa, synthetic construct
DIQMTQSPSTLSASVGDRVTITCRASESISNWLAWYQQKPGKAPKLLIYKAYSLEYGVPSRFSGSGSGTEFTLTISSLQPDDFATYYCQQFQKLPPFTFGGGTKVEIK

SEQ ID NO: 191 - protein, 10 aa, synthetic construct
GYTFRSYGIS

SEQ ID NO: 192 - protein, 17 aa, synthetic construct
WVAPYNGNTNYAQKLQG

SEQ ID NO: 193 - protein, 14 aa, synthetic construct
DAGTYSPYGYGMDV

SEQ ID NO: 194 - protein, 11 aa, synthetic construct
RASQSISSWLA

SEQ ID NO: 195 - protein, 7 aa, synthetic construct
KASSLES

SEQ ID NO: 196 - protein, 10 aa, synthetic construct
QQFQSLPPFT

SEQ ID NO: 197 - protein, 5 aa, synthetic construct
SYGIS

SEQ ID NO: 198 - protein, 17 aa, synthetic construct
WVAPYNGNTNYAQKLQG

SEQ ID NO: 199 - protein, 14 aa, synthetic construct
DAGTYSPYGYGMDV

SEQ ID NO: 200 - protein, 11 aa, synthetic construct
RASQSISSWLA

SEQ ID NO: 201 - protein, 7 aa, synthetic construct
KASSLES

SEQ ID NO: 202 - protein, 10 aa, synthetic construct
QQFQSLPPFT

SEQ ID NO: 203 - protein, 7 aa, synthetic construct
GYTFRSY

SEQ ID NO: 204 - protein, 4 aa, synthetic construct
PYNG

SEQ ID NO: 205 - protein, 12 aa, synthetic construct
AGTYSPYGYGMD

SEQ ID NO: 206 - protein, 7 aa, synthetic construct
SQSISSW

SEQ ID NO: 207 - skipped

SEQ ID NO: 208 - protein, 7 aa, synthetic construct
FQSLPPF

SEQ ID NO: 209 - protein, 10 aa, synthetic construct
GYTFRSYGIS

SEQ ID NO: 210 - protein, 10 aa, synthetic construct
WVAPYNGNTN

SEQ ID NO: 211 - protein, 14 aa, synthetic construct
DAGTYSPYGYGMDV

SEQ ID NO: 212 - protein, 11 aa, synthetic construct
RASQSISSWLA

SEQ ID NO: 213 - protein, 7 aa, synthetic construct
KASSLES

SEQ ID NO: 214 - protein, 10 aa, synthetic construct
QQFQSLPPFT

SEQ ID NO: 215 - protein, 6 aa, synthetic construct
RSYGIS

SEQ ID NO: 216 - protein, 13 aa, synthetic construct
WMGWVAPYNGNTN

SEQ ID NO: 217 - protein, 15 aa, synthetic construct
ARDAGTYSPYGYGMD

SEQ ID NO: 218 - protein, 7 aa, synthetic construct
SSWLAWY

SEQ ID NO: 219 - protein, 10 aa, synthetic construct
LLIYKASSLE

SEQ ID NO: 220 - protein, 9 aa, synthetic construct
QQFQSLPPF

SEQ ID NO: 221 - protein, 8 aa, synthetic construct
GYTFRSYG

SEQ ID NO: 222 - protein, 8 aa, synthetic construct
VAPYNGNT

SEQ ID NO: 223 - protein, 16 aa, synthetic construct
ARDAGTYSPYGYGMDV

SEQ ID NO: 224 - protein, 6 aa, synthetic construct
QSISSW

SEQ ID NO: 225 - skipped

SEQ ID NO: 226 - protein, 10 aa, synthetic construct
QQFQSLPPFT

SEQ ID NO: 227 - protein, 123 aa, synthetic construct
QVQLVQSGAEVKKPGASVKVSCKASGYTFRSYGISWVRQAPGQGLEWMGWVAPYNGNTNYAQKLQGRVTMTTDTSTSTAYMELRSLRSDDTAVYYCARDAGTYSPYGYGMDVWGQGTTVTVSS

SEQ ID NO: 228 - protein, 108 aa, synthetic construct; identical to SEQ ID NO: 114

SEQ ID NO: 229 - protein, 10 aa, synthetic construct
GYTFRSYGIS

SEQ ID NO: 230 - protein, 17 aa, synthetic construct
WVAPYSGNTNYAQKLQG

SEQ ID NO: 231 - protein, 14 aa, synthetic construct
DAGTYSPYGYGMDV

SEQ ID NO: 232 - protein, 11 aa, synthetic construct
RASHSIDSWLA

SEQ ID NO: 233 - protein, 7 aa, synthetic construct
KASYLES

SEQ ID NO: 234 - protein, 10 aa, synthetic construct
QLFQSLPPFT

SEQ ID NO: 235 - protein, 5 aa, synthetic construct
SYGIS

SEQ ID NO: 236 - protein, 17 aa, synthetic construct
WVAPYSGNTNYAQKLQG

SEQ ID NO: 237 - protein, 14 aa, synthetic construct
DAGTYSPYGYGMDV

SEQ ID NO: 238 - protein, 11 aa, synthetic construct
RASHSIDSWLA

SEQ ID NO: 239 - protein, 7 aa, synthetic construct
KASYLES

SEQ ID NO: 240 - protein, 10 aa, synthetic construct
QLFQSLPPFT

SEQ ID NO: 241 - protein, 7 aa, synthetic construct
GYTFRSY

SEQ ID NO: 242 - protein, 4 aa, synthetic construct
PYSG

SEQ ID NO: 243 - protein, 12 aa, synthetic construct
AGTYSPYGYGMD

SEQ ID NO: 244 - protein, 7 aa, synthetic construct
SHSIDSW

SEQ ID NO: 245 - skipped

SEQ ID NO: 246 - protein, 7 aa, synthetic construct
FQSLPPF

SEQ ID NO: 247 - protein, 10 aa, synthetic construct
GYTFRSYGIS

SEQ ID NO: 248 - protein, 10 aa, synthetic construct
WVAPYSGNTN

SEQ ID NO: 249 - protein, 14 aa, synthetic construct
DAGTYSPYGYGMDV

SEQ ID NO: 250 - protein, 11 aa, synthetic construct
RASHSIDSWLA

SEQ ID NO: 251 - protein, 7 aa, synthetic construct
KASYLES

SEQ ID NO: 252 - protein, 10 aa, synthetic construct
QLFQSLPPFT

SEQ ID NO: 253 - protein, 6 aa, synthetic construct
RSYGIS

SEQ ID NO: 254 - protein, 13 aa, synthetic construct
WMGWVAPYSGNTN

SEQ ID NO: 255 - protein, 15 aa, synthetic construct
ARDAGTYSPYGYGMD

SEQ ID NO: 256 - protein, 7 aa, synthetic construct
DSWLAWY

SEQ ID NO: 257 - protein, 10 aa, synthetic construct
LLIYKASYLE

SEQ ID NO: 258 - protein, 9 aa, synthetic construct
QLFQSLPPF

SEQ ID NO: 259 - protein, 8 aa, synthetic construct
GYTFRSYG

SEQ ID NO: 260 - protein, 8 aa, synthetic construct
VAPYSGNT

SEQ ID NO: 261 - protein, 16 aa, synthetic construct
ARDAGTYSPYGYGMDV

SEQ ID NO: 262 - protein, 6 aa, synthetic construct
HSIDSW

SEQ ID NO: 263 - skipped

SEQ ID NO: 264 - protein, 10 aa, synthetic construct
QLFQSLPPFT

SEQ ID NO: 265 - protein, 123 aa, synthetic construct
QVQLVQSGAEVKKPGASVKVSCKASGYTFRSYGISWVRQAPGQGLEWMGWVAPYSGNTNYAQKLQGRVTMTTDTSTSTAYMELRSLRSDDTAVYYCARDAGTYSPYGYGMDVWGQGTTVTVSS

SEQ ID NO: 266 - protein, 108 aa, synthetic construct
DIQMTQSPSTLSASVGDRVTITCRASHSIDSWLAWYQQKPGKAPKLLIYKASYLESGVPSRFSGSGSGTEFTLTISSLQPDDFATYYCQLFQSLPPFTFGGGTKVEIK

SEQ ID NO: 267 - protein, 10 aa, synthetic construct
GYTFRSYGIS

SEQ ID NO: 268 - protein, 17 aa, synthetic construct
WVAPYSGNTNYAQKLQG

SEQ ID NO: 269 - protein, 14 aa, synthetic construct
DAGTYSPYGYGMDV

SEQ ID NO: 270 - protein, 11 aa, synthetic construct
QASQSIDSWLA

SEQ ID NO: 271 - protein, 7 aa, synthetic construct
SASYLES

SEQ ID NO: 272 - protein, 10 aa, synthetic construct
QRFQSLPPFT

SEQ ID NO: 273 - protein, 5 aa, synthetic construct
SYGIS

SEQ ID NO: 274 - protein, 17 aa, synthetic construct
WVAPYSGNTNYAQKLQG

SEQ ID NO: 275 - protein, 14 aa, synthetic construct
DAGTYSPYGYGMDV

SEQ ID NO: 276 - protein, 11 aa, synthetic construct
QASQSIDSWLA

SEQ ID NO: 277 - protein, 7 aa, synthetic construct
SASYLES

SEQ ID NO: 278 - protein, 10 aa, synthetic construct
QRFQSLPPFT

SEQ ID NO: 279 - protein, 7 aa, synthetic construct
GYTFRSY

SEQ ID NO: 280 - protein, 4 aa, synthetic construct
PYSG

SEQ ID NO: 281 - protein, 12 aa, synthetic construct
AGTYSPYGYGMD

SEQ ID NO: 282 - protein, 7 aa, synthetic construct
SQSIDSW

SEQ ID NO: 283 - skipped

SEQ ID NO: 284 - protein, 7 aa, synthetic construct
FQSLPPF

SEQ ID NO: 285 - protein, 10 aa, synthetic construct
GYTFRSYGIS

SEQ ID NO: 286 - protein, 10 aa, synthetic construct
WVAPYSGNTN

SEQ ID NO: 287 - protein, 14 aa, synthetic construct
DAGTYSPYGYGMDV

SEQ ID NO: 288 - protein, 11 aa, synthetic construct
QASQSIDSWLA

SEQ ID NO: 289 - protein, 7 aa, synthetic construct
SASYLES

SEQ ID NO: 290 - protein, 10 aa, synthetic construct
QRFQSLPPFT

SEQ ID NO: 291 - protein, 6 aa, synthetic construct
RSYGIS

SEQ ID NO: 292 - protein, 13 aa, synthetic construct
WMGWVAPYSGNTN

SEQ ID NO: 293 - protein, 15 aa, synthetic construct
ARDAGTYSPYGYGMD

SEQ ID NO: 294 - protein, 7 aa, synthetic construct
DSWLAWY

SEQ ID NO: 295 - protein, 10 aa, synthetic construct
LLIYSASYLE

SEQ ID NO: 296 - protein, 9 aa, synthetic construct
QRFQSLPPF

SEQ ID NO: 297 - protein, 8 aa, synthetic construct
GYTFRSYG

SEQ ID NO: 298 - protein, 8 aa, synthetic construct
VAPYSGNT

SEQ ID NO: 299 - protein, 16 aa, synthetic construct
ARDAGTYSPYGYGMDV

SEQ ID NO: 300 - protein, 6 aa, synthetic construct
QSIDSW

SEQ ID NO: 301 - skipped

SEQ ID NO: 302 - protein, 10 aa, synthetic construct
QRFQSLPPFT

SEQ ID NO: 303 - protein, 123 aa, synthetic construct; identical to SEQ ID NO: 265

SEQ ID NO: 304 - protein, 108 aa, synthetic construct
DIQMTQSPSTLSASVGDRVTITCQASQSIDSWLAWYQQKPGKAPKLLIYSASYLESGVPSRFSGSGSGTEFTLTISSLQPDDFATYYCQRFQSLPPFTFGGGTKVEIK

SEQ ID NO: 305 - protein, 10 aa, synthetic construct
GFTFHSRGMH

SEQ ID NO: 306 - protein, 17 aa, synthetic construct
VITYDGINKYYADSVEG

SEQ ID NO: 307 - protein, 10 aa, synthetic construct
DGVYYGVYDY

SEQ ID NO: 308 - protein, 17 aa, synthetic construct
KSSQSVLFSSNNKNYLA

SEQ ID NO: 309 - protein, 7 aa, synthetic construct
WASTRES

SEQ ID NO: 310 - protein, 9 aa, synthetic construct
QQFHSYPLT

SEQ ID NO: 311 - protein, 5 aa, synthetic construct
SRGMH

SEQ ID NO: 312 - protein, 17 aa, synthetic construct
VITYDGINKYYADSVEG

SEQ ID NO: 313 - protein, 10 aa, synthetic construct
DGVYYGVYDY

SEQ ID NO: 314 - protein, 17 aa, synthetic construct
KSSQSVLFSSNNKNYLA

SEQ ID NO: 315 - protein, 7 aa, synthetic construct
WASTRES

SEQ ID NO: 316 - protein, 9 aa, synthetic construct
QQFHSYPLT

SEQ ID NO: 317 - protein, 7 aa, synthetic construct
GFTFHSR

SEQ ID NO: 318 - protein, 4 aa, synthetic construct
YDGI

SEQ ID NO: 319 - protein, 8 aa, synthetic construct
GVYYGVYD

SEQ ID NO: 320 - protein, 13 aa, synthetic construct
SQSVLFSSNNKNY

SEQ ID NO: 321 - skipped

SEQ ID NO: 322 - protein, 6 aa, synthetic construct
FHSYPL

SEQ ID NO: 323 - protein, 10 aa, synthetic construct
GFTFHSRGMH

SEQ ID NO: 324 - protein, 10 aa, synthetic construct
VITYDGINKY

SEQ ID NO: 325 - protein, 10 aa, synthetic construct
DGVYYGVYDY

SEQ ID NO: 326 - protein, 17 aa, synthetic construct
KSSQSVLFSSNNKNYLA

SEQ ID NO: 327 - protein, 7 aa, synthetic construct
WASTRES

SEQ ID NO: 328 - protein, 9 aa, synthetic construct
QQFHSYPLT

SEQ ID NO: 329 - protein, 6 aa, synthetic construct
HSRGMH

SEQ ID NO: 330 - protein, 13 aa, synthetic construct
WVAVITYDGINKY

SEQ ID NO: 331 - protein, 11 aa, synthetic construct
ARDGVYYGVYD

SEQ ID NO: 332 - protein, 13 aa, synthetic construct
LFSSNNKNYLAWY

SEQ ID NO: 333 - protein, 10 aa, synthetic construct
LLIYWASTRE

SEQ ID NO: 334 - protein, 8 aa, synthetic construct
QQFHSYPL

SEQ ID NO: 335 - protein, 8 aa, synthetic construct
GFTFHSRG

SEQ ID NO: 336 - protein, 8 aa, synthetic construct
ITYDGINK

SEQ ID NO: 337 - protein, 12 aa, synthetic construct
ARDGVYYGVYDY

SEQ ID NO: 338 - protein, 12 aa, synthetic construct
QSVLFSSNNKNY

SEQ ID NO: 339 - skipped

SEQ ID NO: 340 - protein, 9 aa, synthetic construct
QQFHSYPLT

SEQ ID NO: 341 - protein, 119 aa, synthetic construct
QVQLVESGGGVVQPGRSLRLSCAASGFTFHSRGMHWVRQAPGKGLEWVAVITYDGINKYYADSVEGRFTISRDNSKNTLYLQMNSLRAEDTAVYYCARDGVYYGVYDYWGQGTLVTVSS

SEQ ID NO: 342 - protein, 113 aa, synthetic construct
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQFHSYPLTFGGGTKVEIK

SEQ ID NO: 343 - protein, 10 aa, synthetic construct
GFTFRSYGMH

SEQ ID NO: 344 - protein, 17 aa, synthetic construct
VITYDGINKYYADSVEG

SEQ ID NO: 345 - protein, 10 aa, synthetic construct
DGVYYGVYDY

SEQ ID NO: 346 - protein, 17 aa, synthetic construct
KSSQSVLFSSNNKNYLA

SEQ ID NO: 347 - protein, 7 aa, synthetic construct
WASTRES

SEQ ID NO: 348 - protein, 9 aa, synthetic construct
QQFHSYPLT

SEQ ID NO: 349 - protein, 5 aa, synthetic construct
SYGMH

SEQ ID NO: 350 - protein, 17 aa, synthetic construct
VITYDGINKYYADSVEG

SEQ ID NO: 351 - protein, 10 aa, synthetic construct
DGVYYGVYDY

SEQ ID NO: 352 - protein, 17 aa, synthetic construct
KSSQSVLFSSNNKNYLA

SEQ ID NO: 353 - protein, 7 aa, synthetic construct
WASTRES

SEQ ID NO: 354 - protein, 9 aa, synthetic construct
QQFHSYPLT

SEQ ID NO: 355 - protein, 7 aa, synthetic construct
GFTFRSY

SEQ ID NO: 356 - protein, 4 aa, synthetic construct
YDGI

SEQ ID NO: 357 - protein, 8 aa, synthetic construct
GVYYGVYD

SEQ ID NO: 358 - protein, 13 aa, synthetic construct
SQSVLFSSNNKNY

SEQ ID NO: 359 - skipped

SEQ ID NO: 360 - protein, 6 aa, synthetic construct
FHSYPL

SEQ ID NO: 361 - protein, 10 aa, synthetic construct
GFTFRSYGMH

SEQ ID NO: 362 - protein, 10 aa, synthetic construct
VITYDGINKY

SEQ ID NO: 363 - protein, 10 aa, synthetic construct
DGVYYGVYDY

SEQ ID NO: 364 - protein, 17 aa, synthetic construct
KSSQSVLFSSNNKNYLA

SEQ ID NO: 365 - protein, 7 aa, synthetic construct
WASTRES

SEQ ID NO: 366 - protein, 9 aa, synthetic construct
QQFHSYPLT

SEQ ID NO: 367 - protein, 6 aa, synthetic construct
RSYGMH

SEQ ID NO: 368 - protein, 13 aa, synthetic construct
WVAVITYDGINKY

SEQ ID NO: 369 - protein, 11 aa, synthetic construct
ARDGVYYGVYD

SEQ ID NO: 370 - protein, 13 aa, synthetic construct
LFSSNNKNYLAWY

SEQ ID NO: 371 - protein, 10 aa, synthetic construct
LLIYWASTRE

SEQ ID NO: 372 - protein, 8 aa, synthetic construct
QQFHSYPL

SEQ ID NO: 373 - protein, 8 aa, synthetic construct
GFTFRSYG

SEQ ID NO: 374 - protein, 8 aa, synthetic construct
ITYDGINK

SEQ ID NO: 375 - protein, 12 aa, synthetic construct
ARDGVYYGVYDY

SEQ ID NO: 376 - protein, 12 aa, synthetic construct
QSVLFSSNNKNY

SEQ ID NO: 377 - skipped

SEQ ID NO: 378 - protein, 9 aa, synthetic construct
QQFHSYPLT

SEQ ID NO: 379 - protein, 119 aa, synthetic construct
QVQLVESGGGVVQPGRSLRLSCAASGFTFRSYGMHWVRQAPGKGLEWVAVITYDGINKYYADSVEGRFTISRDNSKNTLYLQMNSLRAEDTAVYYCARDGVYYGVYDYWGQGTLVTVSS

SEQ ID NO: 380 - protein, 113 aa, synthetic construct; identical to SEQ ID NO: 342

SEQ ID NO: 381 - protein, 10 aa, synthetic construct
GGTFSSNAIG

SEQ ID NO: 382 - protein, 17 aa, synthetic construct
SIIPIIGFANYAQKFQG

SEQ ID NO: 383 - protein, 15 aa, synthetic construct
DSGYYYGASSFGMDV

SEQ ID NO: 384 - protein, 11 aa, synthetic construct
RASQSVSSNLA

SEQ ID NO: 385 - protein, 7 aa, synthetic construct
GASTRAT

SEQ ID NO: 386 - protein, 9 aa, synthetic construct
EQYNNLPLT

SEQ ID NO: 387 - protein, 5 aa, synthetic construct
SNAIG

SEQ ID NO: 388 - protein, 17 aa, synthetic construct
SIIPIIGFANYAQKFQG

SEQ ID NO: 389 - protein, 15 aa, synthetic construct
DSGYYYGASSFGMDV

SEQ ID NO: 390 - protein, 11 aa, synthetic construct
RASQSVSSNLA

SEQ ID NO: 391 - protein, 7 aa, synthetic construct
GASTRAT

SEQ ID NO: 392 - protein, 9 aa, synthetic construct
EQYNNLPLT

SEQ ID NO: 393 - protein, 7 aa, synthetic construct
GGTFSSN

SEQ ID NO: 394 - protein, 4 aa, synthetic construct
PIIG

SEQ ID NO: 395 - protein, 13 aa, synthetic construct
SGYYYGASSFGMD

SEQ ID NO: 396 - protein, 7 aa, synthetic construct
SQSVSSN

SEQ ID NO: 397 - skipped

SEQ ID NO: 398 - protein, 6 aa, synthetic construct
YNNLPL

SEQ ID NO: 399 - protein, 10 aa, synthetic construct
GGTFSSNAIG

SEQ ID NO: 400 - protein, 10 aa, synthetic construct
SIIPIIGFAN

SEQ ID NO: 401 - protein, 15 aa, synthetic construct
DSGYYYGASSFGMDV

SEQ ID NO: 402 - protein, 11 aa, synthetic construct
RASQSVSSNLA

SEQ ID NO: 403 - protein, 7 aa, synthetic construct
GASTRAT

SEQ ID NO: 404 - protein, 9 aa, synthetic construct
EQYNNLPLT

SEQ ID NO: 405 - protein, 6 aa, synthetic construct
SSNAIG

SEQ ID NO: 406 - protein, 13 aa, synthetic construct
WMGSIIPIIGFAN

SEQ ID NO: 407 - protein, 16 aa, synthetic construct
ARDSGYYYGASSFGMD

SEQ ID NO: 408 - protein, 7 aa, synthetic construct
SSNLAWY

SEQ ID NO: 409 - protein, 10 aa, synthetic construct
LLIYGASTRA

SEQ ID NO: 410 - protein, 8 aa, synthetic construct
EQYNNLPL

SEQ ID NO: 411 - protein, 8 aa, synthetic construct
GGTFSSNA

SEQ ID NO: 412 - protein, 8 aa, synthetic construct
IIPIIGFA

SEQ ID NO: 413 - protein, 17 aa, synthetic construct
ARDSGYYYGASSFGMDV

SEQ ID NO: 414 - protein, 6 aa, synthetic construct
QSVSSN

SEQ ID NO: 415 - skipped

SEQ ID NO: 416 - protein, 9 aa, synthetic construct
EQYNNLPLT

SEQ ID NO: 417 - protein, 124 aa, synthetic construct
QVQLVQSGAEVKKPGSSVKVSCKASGGTFSSNAIGWVRQAPGQGLEWMGSIIPIIGFANYAQKFQGRVTITADESTSTAYMELSSLRSEDTAVYYCARDSGYYYGASSFGMDVWGQGTTVTVSS

SEQ ID NO: 418 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCEQYNNLPLTFGGGTKVEIK

SEQ ID NO: 419 - protein, 11 aa, synthetic construct
GGSISSGQYWS

SEQ ID NO: 420 - protein, 16 aa, synthetic construct
EIYYSGSTRYNPSLKS

SEQ ID NO: 421 - protein, 16 aa, synthetic construct
DAPYYYGGGYYYYMDV

SEQ ID NO: 422 - protein, 12 aa, synthetic construct
RASQSVSSSYLA

SEQ ID NO: 423 - protein, 7 aa, synthetic construct
GASSRAT

SEQ ID NO: 424 - protein, 9 aa, synthetic construct
QQVGVVPYT

SEQ ID NO: 425 - protein, 6 aa, synthetic construct
SGQYWS

SEQ ID NO: 426 - protein, 16 aa, synthetic construct
EIYYSGSTRYNPSLKS

SEQ ID NO: 427 - protein, 16 aa, synthetic construct
DAPYYYGGGYYYYMDV

SEQ ID NO: 428 - protein, 12 aa, synthetic construct
RASQSVSSSYLA

SEQ ID NO: 429 - protein, 7 aa, synthetic construct
GASSRAT

SEQ ID NO: 430 - protein, 9 aa, synthetic construct
QQVGVVPYT

SEQ ID NO: 431 - protein, 8 aa, synthetic construct
GGSISSGQ

SEQ ID NO: 432 - skipped

SEQ ID NO: 433 - protein, 14 aa, synthetic construct
APYYYGGGYYYYMD

SEQ ID NO: 434 - protein, 8 aa, synthetic construct
SQSVSSSY

SEQ ID NO: 435 - skipped

SEQ ID NO: 436 - protein, 6 aa, synthetic construct
VGVVPY

SEQ ID NO: 437 - protein, 11 aa, synthetic construct
GGSISSGQYWS

SEQ ID NO: 438 - protein, 9 aa, synthetic construct
EIYYSGSTR

SEQ ID NO: 439 - protein, 16 aa, synthetic construct
DAPYYYGGGYYYYMDV

SEQ ID NO: 440 - protein, 12 aa, synthetic construct
RASQSVSSSYLA

SEQ ID NO: 441 - protein, 7 aa, synthetic construct
GASSRAT

SEQ ID NO: 442 - protein, 9 aa, synthetic construct
QQVGVVPYT

SEQ ID NO: 443 - protein, 7 aa, synthetic construct
SSGQYWS

SEQ ID NO: 444 - protein, 12 aa, synthetic construct
WIGEIYYSGSTR

SEQ ID NO: 445 - protein, 17 aa, synthetic construct
ARDAPYYYGGGYYYYMD

SEQ ID NO: 446 - protein, 8 aa, synthetic construct
SSSYLAWY

SEQ ID NO: 447 - protein, 10 aa, synthetic construct
LLIYGASSRA

SEQ ID NO: 448 - protein, 8 aa, synthetic construct
QQVGVVPY

SEQ ID NO: 449 - protein, 9 aa, synthetic construct
GGSISSGQY

SEQ ID NO: 450 - protein, 7 aa, synthetic construct
IYYSGST

SEQ ID NO: 451 - protein, 18 aa, synthetic construct
ARDAPYYYGGGYYYYMDV

SEQ ID NO: 452 - protein, 7 aa, synthetic construct
QSVSSSY

SEQ ID NO: 453 - skipped

SEQ ID NO: 454 - protein, 9 aa, synthetic construct
QQVGVVPYT

SEQ ID NO: 455 - protein, 125 aa, synthetic construct
QVQLQESGPGLVKPSQTLSLTCTVSGGSISSGQYWSWIRQHPGKGLEWIGEIYYSGSTRYNPSLKSRVTISVDTSKNQFSLKLSSVTAADTAVYYCARDAPYYYGGGYYYYMDVWGKGTTVTVSS

SEQ ID NO: 456 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQVGVVPYTFGGGTKVEIK

SEQ ID NO: 457 - protein, 11 aa, synthetic construct
GGSISSGQYWS

SEQ ID NO: 458 - protein, 16 aa, synthetic construct
EIYYSGSTRYNPSLKS

SEQ ID NO: 459 - protein, 16 aa, synthetic construct
DAPYYYGGGYYYYMDV

SEQ ID NO: 460 - protein, 12 aa, synthetic construct
RASESVDSSYLA

SEQ ID NO: 461 - protein, 7 aa, synthetic construct
GASTRQT

SEQ ID NO: 462 - protein, 9 aa, synthetic construct
QQAGVVPYT

SEQ ID NO: 463 - protein, 6 aa, synthetic construct
SGQYWS

SEQ ID NO: 464 - protein, 16 aa, synthetic construct
EIYYSGSTRYNPSLKS

SEQ ID NO: 465 - protein, 16 aa, synthetic construct
DAPYYYGGGYYYYMDV

SEQ ID NO: 466 - protein, 12 aa, synthetic construct
RASESVDSSYLA

SEQ ID NO: 467 - protein, 7 aa, synthetic construct
GASTRQT

SEQ ID NO: 468 - protein, 9 aa, synthetic construct
QQAGVVPYT

SEQ ID NO: 469 - protein, 8 aa, synthetic construct
GGSISSGQ

SEQ ID NO: 470 - skipped

SEQ ID NO: 471 - protein, 14 aa, synthetic construct
APYYYGGGYYYYMD

SEQ ID NO: 472 - protein, 8 aa, synthetic construct
SESVDSSY

SEQ ID NO: 473 - skipped

SEQ ID NO: 474 - protein, 6 aa, synthetic construct
AGVVPY

SEQ ID NO: 475 - protein, 11 aa, synthetic construct
GGSISSGQYWS

SEQ ID NO: 476 - protein, 9 aa, synthetic construct
EIYYSGSTR

SEQ ID NO: 477 - protein, 16 aa, synthetic construct
DAPYYYGGGYYYYMDV

SEQ ID NO: 478 - protein, 12 aa, synthetic construct
RASESVDSSYLA

SEQ ID NO: 479 - protein, 7 aa, synthetic construct
GASTRQT

SEQ ID NO: 480 - protein, 9 aa, synthetic construct
QQAGVVPYT

SEQ ID NO: 481 - protein, 7 aa, synthetic construct
SSGQYWS

SEQ ID NO: 482 - protein, 12 aa, synthetic construct
WIGEIYYSGSTR

SEQ ID NO: 483 - protein, 17 aa, synthetic construct
ARDAPYYYGGGYYYYMD

SEQ ID NO: 484 - protein, 8 aa, synthetic construct
DSSYLAWY

SEQ ID NO: 485 - protein, 10 aa, synthetic construct
LLIYGASTRQ

SEQ ID NO: 486 - protein, 8 aa, synthetic construct
QQAGVVPY

SEQ ID NO: 487 - protein, 9 aa, synthetic construct
GGSISSGQY

SEQ ID NO: 488 - protein, 7 aa, synthetic construct
IYYSGST

SEQ ID NO: 489 - protein, 18 aa, synthetic construct
ARDAPYYYGGGYYYYMDV

SEQ ID NO: 490 - protein, 7 aa, synthetic construct
ESVDSSY

SEQ ID NO: 491 - skipped

SEQ ID NO: 492 - protein, 9 aa, synthetic construct
QQAGVVPYT

SEQ ID NO: 493 - protein, 125 aa, synthetic construct; identical to SEQ ID NO: 455

SEQ ID NO: 494 - protein, 108 aa, synthetic construct
EIVLTQSPGTLSLSPGERATLSCRASESVDSSYLAWYQQKPGQAPRLLIYGASTRQTGIPDRFSGSGSGTDFTLTISRLEPEDFAVYYCQQAGVVPYTFGGGTKVEIK

SEQ ID NO: 495 - protein, 11 aa, synthetic construct
GGSISSGQYWS

SEQ ID NO: 496 - protein, 16 aa, synthetic construct
EIYYSGSTRYNPSLKS

SEQ ID NO: 497 - protein, 16 aa, synthetic construct
DAPYYYGGGYYYYMDV

SEQ ID NO: 498 - protein, 12 aa, synthetic construct
RASESVDSSYLA

SEQ ID NO: 499 - protein, 7 aa, synthetic construct
GADSRAT

SEQ ID NO: 500 - protein, 9 aa, synthetic construct
QQDGVVPYT

SEQ ID NO: 501 - protein, 6 aa, synthetic construct
SGQYWS

SEQ ID NO: 502 - protein, 16 aa, synthetic construct
EIYYSGSTRYNPSLKS

SEQ ID NO: 503 - protein, 16 aa, synthetic construct
DAPYYYGGGYYYYMDV

SEQ ID NO: 504 - protein, 12 aa, synthetic construct
RASESVDSSYLA

SEQ ID NO: 505 - protein, 7 aa, synthetic construct
GADSRAT

SEQ ID NO: 506 - protein, 9 aa, synthetic construct
QQDGVVPYT

SEQ ID NO: 507 - protein, 8 aa, synthetic construct
GGSISSGQ

SEQ ID NO: 508 - skipped

SEQ ID NO: 509 - protein, 14 aa, synthetic construct
APYYYGGGYYYYMD

SEQ ID NO: 510 - protein, 8 aa, synthetic construct
SESVDSSY

SEQ ID NO: 511 - skipped

SEQ ID NO: 512 - protein, 6 aa, synthetic construct
DGVVPY

SEQ ID NO: 513 - protein, 11 aa, synthetic construct
GGSISSGQYWS

SEQ ID NO: 514 - protein, 9 aa, synthetic construct
EIYYSGSTR

SEQ ID NO: 515 - protein, 16 aa, synthetic construct
DAPYYYGGGYYYYMDV

SEQ ID NO: 516 - protein, 12 aa, synthetic construct
RASESVDSSYLA

SEQ ID NO: 517 - protein, 7 aa, synthetic construct
GADSRAT

SEQ ID NO: 518 - protein, 9 aa, synthetic construct
QQDGVVPYT

SEQ ID NO: 519 - protein, 7 aa, synthetic construct
SSGQYWS

SEQ ID NO: 520 - protein, 12 aa, synthetic construct
WIGEIYYSGSTR

SEQ ID NO: 521 - protein, 17 aa, synthetic construct
ARDAPYYYGGGYYYYMD

SEQ ID NO: 522 - protein, 8 aa, synthetic construct
DSSYLAWY

SEQ ID NO: 523 - protein, 10 aa, synthetic construct
LLIYGADSRA

SEQ ID NO: 524 - protein, 8 aa, synthetic construct
QQDGVVPY

SEQ ID NO: 525 - protein, 9 aa, synthetic construct
GGSISSGQY

SEQ ID NO: 526 - protein, 7 aa, synthetic construct
IYYSGST

SEQ ID NO: 527 - protein, 18 aa, synthetic construct
ARDAPYYYGGGYYYYMDV

SEQ ID NO: 528 - protein, 7 aa, synthetic construct
ESVDSSY

SEQ ID NO: 529 - skipped

SEQ ID NO: 530 - protein, 9 aa, synthetic construct
QQDGVVPYT

SEQ ID NO: 531 - protein, 125 aa, synthetic construct; identical to SEQ ID NO: 455

SEQ ID NO: 532 - protein, 108 aa, synthetic construct
EIVLTQSPGTLSLSPGERATLSCRASESVDSSYLAWYQQKPGQAPRLLIYGADSRATGIPDRFSGSGSGTDFTLTISRLEPEDFAVYYCQQDGVVPYTFGGGTKVEIK

SEQ ID NO: 533 - protein, 10 aa, synthetic construct
GGSLSGYYWS

SEQ ID NO: 534 - protein, 16 aa, synthetic construct
EIGASGSTRYNPSLKS

SEQ ID NO: 535 - protein, 16 aa, synthetic construct
DTPYYYEGGYYYYMDV

SEQ ID NO: 536 - protein, 12 aa, synthetic construct
RASQSVSSSYLA

SEQ ID NO: 537 - protein, 7 aa, synthetic construct
GASSRAT

SEQ ID NO: 538 - protein, 9 aa, synthetic construct
QQVGVVPYT

SEQ ID NO: 539 - protein, 5 aa, synthetic construct
GYYWS

SEQ ID NO: 540 - protein, 16 aa, synthetic construct
EIGASGSTRYNPSLKS

SEQ ID NO: 541 - protein, 16 aa, synthetic construct
DTPYYYEGGYYYYMDV

SEQ ID NO: 542 - protein, 12 aa, synthetic construct
RASQSVSSSYLA

SEQ ID NO: 543 - protein, 7 aa, synthetic construct
GASSRAT

SEQ ID NO: 544 - protein, 9 aa, synthetic construct
QQVGVVPYT

SEQ ID NO: 545 - protein, 7 aa, synthetic construct
GGSLSGY

SEQ ID NO: 546 - skipped

SEQ ID NO: 547 - protein, 14 aa, synthetic construct
TPYYYEGGYYYYMD

SEQ ID NO: 548 - protein, 8 aa, synthetic construct
SQSVSSSY

SEQ ID NO: 549 - skipped

SEQ ID NO: 550 - protein, 6 aa, synthetic construct
VGVVPY

SEQ ID NO: 551 - protein, 10 aa, synthetic construct
GGSLSGYYWS

SEQ ID NO: 552 - protein, 9 aa, synthetic construct
EIGASGSTR

SEQ ID NO: 553 - protein, 16 aa, synthetic construct
DTPYYYEGGYYYYMDV

SEQ ID NO: 554 - protein, 12 aa, synthetic construct
RASQSVSSSYLA

SEQ ID NO: 555 - protein, 7 aa, synthetic construct
GASSRAT

SEQ ID NO: 556 - protein, 9 aa, synthetic construct
QQVGVVPYT

SEQ ID NO: 557 - protein, 6 aa, synthetic construct
SGYYWS

SEQ ID NO: 558 - protein, 12 aa, synthetic construct
WIGEIGASGSTR

SEQ ID NO: 559 - protein, 17 aa, synthetic construct
ARDTPYYYEGGYYYYMD

SEQ ID NO: 560 - protein, 8 aa, synthetic construct
SSSYLAWY

SEQ ID NO: 561 - protein, 10 aa, synthetic construct
LLIYGASSRA

SEQ ID NO: 562 - protein, 8 aa, synthetic construct
QQVGVVPY

SEQ ID NO: 563 - protein, 8 aa, synthetic construct
GGSLSGYY

SEQ ID NO: 564 - protein, 7 aa, synthetic construct
IGASGST

SEQ ID NO: 565 - protein, 18 aa, synthetic construct
ARDTPYYYEGGYYYYMDV

SEQ ID NO: 566 - protein, 7 aa, synthetic construct
QSVSSSY

SEQ ID NO: 567 - skipped

SEQ ID NO: 568 - protein, 9 aa, synthetic construct
QQVGVVPYT

SEQ ID NO: 569 - protein, 124 aa, synthetic construct
QVQLQQWGAGLLKPSETLSLTCAVYGGSLSGYYWSWIRQPPGKGLEWIGEIGASGSTRYNPSLKSRVTISVDTSKNQFSLKLSSVTAADTAVYYCARDTPYYYEGGYYYYMDVWGKGTTVTVSS

SEQ ID NO: 570 - protein, 108 aa, synthetic construct; identical to SEQ ID NO: 456

SEQ ID NO: 571 - protein, 10 aa, synthetic construct
GGSLSGYYWS

SEQ ID NO: 572 - protein, 16 aa, synthetic construct
EIGASGSTRYNPSLKS

SEQ ID NO: 573 - protein, 16 aa, synthetic construct
DTPYYYEGGYYYYMDV

SEQ ID NO: 574 - protein, 12 aa, synthetic construct
RASDSVDSSYLA

SEQ ID NO: 575 - protein, 7 aa, synthetic construct
GAFSRAN

SEQ ID NO: 576 - protein, 9 aa, synthetic construct
QQAGVVPYT

SEQ ID NO: 577 - protein, 5 aa, synthetic construct
GYYWS

SEQ ID NO: 578 - protein, 16 aa, synthetic construct
EIGASGSTRYNPSLKS

SEQ ID NO: 579 - protein, 16 aa, synthetic construct
DTPYYYEGGYYYYMDV

SEQ ID NO: 580 - protein, 12 aa, synthetic construct
RASDSVDSSYLA

SEQ ID NO: 581 - protein, 7 aa, synthetic construct
GAFSRAN

SEQ ID NO: 582 - protein, 9 aa, synthetic construct
QQAGVVPYT

SEQ ID NO: 583 - protein, 7 aa, synthetic construct
GGSLSGY

SEQ ID NO: 584 - skipped

SEQ ID NO: 585 - protein, 14 aa, synthetic construct
TPYYYEGGYYYYMD

SEQ ID NO: 586 - protein, 8 aa, synthetic construct
SDSVDSSY

SEQ ID NO: 587 - skipped

SEQ ID NO: 588 - protein, 6 aa, synthetic construct
AGVVPY

SEQ ID NO: 589 - protein, 10 aa, synthetic construct
GGSLSGYYWS

SEQ ID NO: 590 - protein, 9 aa, synthetic construct
EIGASGSTR

SEQ ID NO: 591 - protein, 16 aa, synthetic construct
DTPYYYEGGYYYYMDV

SEQ ID NO: 592 - protein, 12 aa, synthetic construct
RASDSVDSSYLA

SEQ ID NO: 593 - protein, 7 aa, synthetic construct
GAFSRAN

SEQ ID NO: 594 - protein, 9 aa, synthetic construct
QQAGVVPYT

SEQ ID NO: 595 - protein, 6 aa, synthetic construct
SGYYWS

SEQ ID NO: 596 - protein, 12 aa, synthetic construct
WIGEIGASGSTR

SEQ ID NO: 597 - protein, 17 aa, synthetic construct
ARDTPYYYEGGYYYYMD

SEQ ID NO: 598 - protein, 8 aa, synthetic construct
DSSYLAWY

SEQ ID NO: 599 - protein, 10 aa, synthetic construct
LLIYGAFSRA

SEQ ID NO: 600 - protein, 8 aa, synthetic construct
QQAGVVPY

SEQ ID NO: 601 - protein, 8 aa, synthetic construct
GGSLSGYY

SEQ ID NO: 602 - protein, 7 aa, synthetic construct
IGASGST

SEQ ID NO: 603 - protein, 18 aa, synthetic construct
ARDTPYYYEGGYYYYMDV

SEQ ID NO: 604 - protein, 7 aa, synthetic construct
DSVDSSY

SEQ ID NO: 605 - skipped

SEQ ID NO: 606 - protein, 9 aa, synthetic construct
QQAGVVPYT

SEQ ID NO: 607 - protein, 124 aa, synthetic construct; identical to SEQ ID NO: 569

SEQ ID NO: 608 - protein, 108 aa, synthetic construct
EIVLTQSPGTLSLSPGERATLSCRASDSVDSSYLAWYQQKPGQAPRLLIYGAFSRANGIPDRFSGSGSGTDFTLTISRLEPEDFAVYYCQQAGVVPYTFGGGTKVEIK

SEQ ID NO: 609 - protein, 10 aa, synthetic construct
GGSLSGYYWS

SEQ ID NO: 610 - protein, 16 aa, synthetic construct
EIGASGSTRYNPSLKS

SEQ ID NO: 611 - protein, 16 aa, synthetic construct
DTPYYYEGGYYYYMDV

SEQ ID NO: 612 - protein, 12 aa, synthetic construct
RASQSVSSSFLA

SEQ ID NO: 613 - protein, 7 aa, synthetic construct
GAYSRAT

SEQ ID NO: 614 - protein, 9 aa, synthetic construct
QQAGVVPYT

SEQ ID NO: 615 - protein, 5 aa, synthetic construct
GYYWS

SEQ ID NO: 616 - protein, 16 aa, synthetic construct
EIGASGSTRYNPSLKS

SEQ ID NO: 617 - protein, 16 aa, synthetic construct
DTPYYYEGGYYYYMDV

SEQ ID NO: 618 - protein, 12 aa, synthetic construct
RASQSVSSSFLA

SEQ ID NO: 619 - protein, 7 aa, synthetic construct
GAYSRAT

SEQ ID NO: 620 - protein, 9 aa, synthetic construct
QQAGVVPYT

SEQ ID NO: 621 - protein, 7 aa, synthetic construct
GGSLSGY

SEQ ID NO: 622 - skipped

SEQ ID NO: 623 - protein, 14 aa, synthetic construct
TPYYYEGGYYYYMD

SEQ ID NO: 624 - protein, 8 aa, synthetic construct
SQSVSSSF

SEQ ID NO: 625 - skipped

SEQ ID NO: 626 - protein, 6 aa, synthetic construct
AGVVPY

SEQ ID NO: 627 - protein, 10 aa, synthetic construct
GGSLSGYYWS

SEQ ID NO: 628 - protein, 9 aa, synthetic construct
EIGASGSTR

SEQ ID NO: 629 - protein, 16 aa, synthetic construct
DTPYYYEGGYYYYMDV

SEQ ID NO: 630 - protein, 12 aa, synthetic construct
RASQSVSSSFLA

SEQ ID NO: 631 - protein, 7 aa, synthetic construct
GAYSRAT

SEQ ID NO: 632 - protein, 9 aa, synthetic construct
QQAGVVPYT

SEQ ID NO: 633 - protein, 6 aa, synthetic construct
SGYYWS

SEQ ID NO: 634 - protein, 12 aa, synthetic construct
WIGEIGASGSTR

SEQ ID NO: 635 - protein, 17 aa, synthetic construct
ARDTPYYYEGGYYYYMD

SEQ ID NO: 636 - protein, 8 aa, synthetic construct
SSSFLAWY

SEQ ID NO: 637 - protein, 10 aa, synthetic construct
LLIYGAYSRA

SEQ ID NO: 638 - protein, 8 aa, synthetic construct
QQAGVVPY

SEQ ID NO: 639 - protein, 8 aa, synthetic construct
GGSLSGYY

SEQ ID NO: 640 - protein, 7 aa, synthetic construct
IGASGST

SEQ ID NO: 641 - protein, 18 aa, synthetic construct
ARDTPYYYEGGYYYYMDV

SEQ ID NO: 642 - protein, 7 aa, synthetic construct
QSVSSSF

SEQ ID NO: 643 - skipped

SEQ ID NO: 644 - protein, 9 aa, synthetic construct
QQAGVVPYT

SEQ ID NO: 645 - protein, 124 aa, synthetic construct; identical to SEQ ID NO: 569

SEQ ID NO: 646 - protein, 108 aa, synthetic construct
EIVLTQSPGTLSLSPGERATLSCRASQSVSSSFLAWYQQKPGQAPRLLIYGAYSRATGIPDRFSGSGSGTDFTLTISRLEPEDFAVYYCQQAGVVPYTFGGGTKVEIK

SEQ ID NO: 647 - protein, 11 aa, synthetic construct
GGSISSGQYWS

SEQ ID NO: 648 - protein, 16 aa, synthetic construct
EIYYSGSTRYNPSLKS

SEQ ID NO: 649 - protein, 16 aa, synthetic construct
DTPYYYDGGYYYYMDV

SEQ ID NO: 650 - protein, 12 aa, synthetic construct
RASQSVSSSYLA

SEQ ID NO: 651 - protein, 7 aa, synthetic construct
GASSRAT

SEQ ID NO: 652 - protein, 9 aa, synthetic construct
QQVGVVPYT

SEQ ID NO: 653 - protein, 6 aa, synthetic construct
SGQYWS

SEQ ID NO: 654 - protein, 16 aa, synthetic construct
EIYYSGSTRYNPSLKS

SEQ ID NO: 655 - protein, 16 aa, synthetic construct
DTPYYYDGGYYYYMDV

SEQ ID NO: 656 - protein, 12 aa, synthetic construct
RASQSVSSSYLA

SEQ ID NO: 657 - protein, 7 aa, synthetic construct
GASSRAT

SEQ ID NO: 658 - protein, 9 aa, synthetic construct
QQVGVVPYT

SEQ ID NO: 659 - protein, 8 aa, synthetic construct
GGSISSGQ

SEQ ID NO: 660 - skipped

SEQ ID NO: 661 - protein, 14 aa, synthetic construct
TPYYYDGGYYYYMD

SEQ ID NO: 662 - protein, 8 aa, synthetic construct
SQSVSSSY

SEQ ID NO: 663 - skipped

SEQ ID NO: 664 - protein, 6 aa, synthetic construct
VGVVPY

SEQ ID NO: 665 - protein, 11 aa, synthetic construct
GGSISSGQYWS

SEQ ID NO: 666 - protein, 9 aa, synthetic construct
EIYYSGSTR

SEQ ID NO: 667 - protein, 16 aa, synthetic construct
DTPYYYDGGYYYYMDV

SEQ ID NO: 668 - protein, 12 aa, synthetic construct
RASQSVSSSYLA

SEQ ID NO: 669 - protein, 7 aa, synthetic construct
GASSRAT

SEQ ID NO: 670 - protein, 9 aa, synthetic construct
QQVGVVPYT

SEQ ID NO: 671 - protein, 7 aa, synthetic construct
SSGQYWS

SEQ ID NO: 672 - protein, 12 aa, synthetic construct
WIGEIYYSGSTR

SEQ ID NO: 673 - protein, 17 aa, synthetic construct
ARDTPYYYDGGYYYYMD

SEQ ID NO: 674 - protein, 8 aa, synthetic construct
SSSYLAWY

SEQ ID NO: 675 - protein, 10 aa, synthetic construct
LLIYGASSRA

SEQ ID NO: 676 - protein, 8 aa, synthetic construct
QQVGVVPY

SEQ ID NO: 677 - protein, 9 aa, synthetic construct
GGSISSGQY

SEQ ID NO: 678 - protein, 7 aa, synthetic construct
IYYSGST

SEQ ID NO: 679 - protein, 18 aa, synthetic construct
ARDTPYYYDGGYYYYMDV

SEQ ID NO: 680 - protein, 7 aa, synthetic construct
QSVSSSY

SEQ ID NO: 681 - skipped

SEQ ID NO: 682 - protein, 9 aa, synthetic construct
QQVGVVPYT

SEQ ID NO: 683 - protein, 125 aa, synthetic construct
QVQLQESGPGLVKPSQTLSLTCTVSGGSISSGQYWSWIRQHPGKGLEWIGEIYYSGSTRYNPSLKSRVTISVDTSKDQFSLKLSSVTAADTAVYYCARDTPYYYDGGYYYYMDVWGKGTTVTVSS

SEQ ID NO: 684 - protein, 108 aa, synthetic construct; identical to SEQ ID NO: 456

SEQ ID NO: 685 - protein, 11 aa, synthetic construct
GGSISSGQYWS

SEQ ID NO: 686 - protein, 16 aa, synthetic construct
EIYYSGSTRYNPSLKS

SEQ ID NO: 687 - protein, 16 aa, synthetic construct
DTPYYYDGGYYYYMDV

SEQ ID NO: 688 - protein, 12 aa, synthetic construct
RASQSVSSSYLA

SEQ ID NO: 689 - protein, 7 aa, synthetic construct
GASSRAT

SEQ ID NO: 690 - protein, 9 aa, synthetic construct
QQVGVVPYT

SEQ ID NO: 691 - protein, 6 aa, synthetic construct
SGQYWS

SEQ ID NO: 692 - protein, 16 aa, synthetic construct
EIYYSGSTRYNPSLKS

SEQ ID NO: 693 - protein, 16 aa, synthetic construct
DTPYYYDGGYYYYMDV

SEQ ID NO: 694 - protein, 12 aa, synthetic construct
RASQSVSSSYLA

SEQ ID NO: 695 - protein, 7 aa, synthetic construct
GASSRAT

SEQ ID NO: 696 - protein, 9 aa, synthetic construct
QQVGVVPYT

SEQ ID NO: 697 - protein, 8 aa, synthetic construct
GGSISSGQ

SEQ ID NO: 698 - skipped

SEQ ID NO: 699 - protein, 14 aa, synthetic construct
TPYYYDGGYYYYMD

SEQ ID NO: 700 - protein, 8 aa, synthetic construct
SQSVSSSY

SEQ ID NO: 701 - skipped

SEQ ID NO: 702 - protein, 6 aa, synthetic construct
VGVVPY

SEQ ID NO: 703 - protein, 11 aa, synthetic construct
GGSISSGQYWS

SEQ ID NO: 704 - protein, 9 aa, synthetic construct
EIYYSGSTR

SEQ ID NO: 705 - protein, 16 aa, synthetic construct
DTPYYYDGGYYYYMDV

SEQ ID NO: 706 - protein, 12 aa, synthetic construct
RASQSVSSSYLA

SEQ ID NO: 707 - protein, 7 aa, synthetic construct
GASSRAT

SEQ ID NO: 708 - protein, 9 aa, synthetic construct
QQVGVVPYT

SEQ ID NO: 709 - protein, 7 aa, synthetic construct
SSGQYWS

SEQ ID NO: 710 - protein, 12 aa, synthetic construct
WIGEIYYSGSTR

SEQ ID NO: 711 - protein, 17 aa, synthetic construct
ARDTPYYYDGGYYYYMD

SEQ ID NO: 712 - protein, 8 aa, synthetic construct
SSSYLAWY

SEQ ID NO: 713 - protein, 10 aa, synthetic construct
LLIYGASSRA

SEQ ID NO: 714 - protein, 8 aa, synthetic construct
QQVGVVPY

SEQ ID NO: 715 - protein, 9 aa, synthetic construct
GGSISSGQY

SEQ ID NO: 716 - protein, 7 aa, synthetic construct
IYYSGST

SEQ ID NO: 717 - protein, 18 aa, synthetic construct
ARDTPYYYDGGYYYYMDV

SEQ ID NO: 718 - protein, 7 aa, synthetic construct
QSVSSSY

SEQ ID NO: 719 - skipped

SEQ ID NO: 720 - protein, 9 aa, synthetic construct
QQVGVVPYT

SEQ ID NO: 721 - protein, 125 aa, synthetic construct
QVQLQESGPGLVKPSQTLSLTCTVSGGSISSGQYWSWIRQHPGKGLEWIGEIYYSGSTRYNPSLKSRVTISVDTSKNQFSLKLSSVTAADTAVYYCARDTPYYYDGGYYYYMDVWGKGTTVTVSS

SEQ ID NO: 722 - protein, 108 aa, synthetic construct; identical to SEQ ID NO: 456

SEQ ID NO: 723 - protein, 10 aa, synthetic construct
GYTFANYYMH

SEQ ID NO: 724 - protein, 17 aa, synthetic construct
IINPSGGITVYAQKFQG

SEQ ID NO: 725 - protein, 12 aa, synthetic construct
GGSKVAALAFDI

SEQ ID NO: 726 - protein, 11 aa, synthetic construct
QASQDISNSLN

SEQ ID NO: 727 - protein, 7 aa, synthetic construct
DASNLET

SEQ ID NO: 728 - protein, 9 aa, synthetic construct
QQYNFHPLT

SEQ ID NO: 729 - protein, 5 aa, synthetic construct
NYYMH

SEQ ID NO: 730 - protein, 17 aa, synthetic construct
IINPSGGITVYAQKFQG

SEQ ID NO: 731 - protein, 12 aa, synthetic construct
GGSKVAALAFDI

SEQ ID NO: 732 - protein, 11 aa, synthetic construct
QASQDISNSLN

SEQ ID NO: 733 - protein, 7 aa, synthetic construct
DASNLET

SEQ ID NO: 734 - protein, 9 aa, synthetic construct
QQYNFHPLT

SEQ ID NO: 735 - protein, 7 aa, synthetic construct
GYTFANY

SEQ ID NO: 736 - protein, 4 aa, synthetic construct
PSGG

SEQ ID NO: 737 - protein, 10 aa, synthetic construct
GSKVAALAFD

SEQ ID NO: 738 - protein, 7 aa, synthetic construct
SQDISNS

SEQ ID NO: 739 - skipped

SEQ ID NO: 740 - protein, 6 aa, synthetic construct
YNFHPL

SEQ ID NO: 741 - protein, 10 aa, synthetic construct
GYTFANYYMH

SEQ ID NO: 742 - protein, 10 aa, synthetic construct
IINPSGGITV

SEQ ID NO: 743 - protein, 12 aa, synthetic construct
GGSKVAALAFDI

SEQ ID NO: 744 - protein, 11 aa, synthetic construct
QASQDISNSLN

SEQ ID NO: 745 - protein, 7 aa, synthetic construct
DASNLET

SEQ ID NO: 746 - protein, 9 aa, synthetic construct
QQYNFHPLT

SEQ ID NO: 747 - protein, 6 aa, synthetic construct
ANYYMH

SEQ ID NO: 748 - protein, 13 aa, synthetic construct
WMGIINPSGGITV

SEQ ID NO: 749 - protein, 13 aa, synthetic construct
ARGGSKVAALAFD

SEQ ID NO: 750 - protein, 7 aa, synthetic construct
SNSLNWY

SEQ ID NO: 751 - protein, 10 aa, synthetic construct
LLIYDASNLE

SEQ ID NO: 752 - protein, 8 aa, synthetic construct
QQYNFHPL

SEQ ID NO: 753 - protein, 8 aa, synthetic construct
GYTFANYY

SEQ ID NO: 754 - protein, 8 aa, synthetic construct
INPSGGIT

SEQ ID NO: 755 - protein, 14 aa, synthetic construct
ARGGSKVAALAFDI

SEQ ID NO: 756 - protein, 6 aa, synthetic construct
QDISNS

SEQ ID NO: 757 - skipped

SEQ ID NO: 758 - protein, 9 aa, synthetic construct
QQYNFHPLT

SEQ ID NO: 759 - protein, 121 aa, synthetic construct
QVQLVQSGAEVKKPGASVKVSCKASGYTFANYYMHWVRQAPGQGLEWMGIINPSGGITVYAQKFQGRVTMTRDTSTSTVYMELSSLRSEDTAVYYCARGGSKVAALAFDIWGQGTMVTVSS

SEQ ID NO: 760 - protein, 107 aa, synthetic construct
DIQMTQSPSSLSASVGDRVTITCQASQDISNSLNWYQQKPGKAPKLLIYDASNLETGVPSRFSGSRSGTDFTFTISSLQPEDIATYYCQQYNFHPLTFGGGTKVEIK

SEQ ID NO: 761 - protein, 118 aa, synthetic construct
  VARIANT 31: note = D or S
  VARIANT 35: note = A or G
  VARIANT 50: note = A or T
EVQLLESGGGLVQPGGSLRLSCAASGFTFSXYAMXWVRQAPGKGLEWVSXISGSGGLTYYADSVKGRFTISRDNSKNTLYLQMNSLRAEDTAVYYCAKAPYGYYMDVWGKGTTVTVSS

SEQ ID NO: 762 - protein, 106 aa, synthetic construct; identical to SEQ ID NO: 38

SEQ ID NO: 763 - protein, 123 aa, synthetic construct
  VARIANT 30: note = D or R
  VARIANT 31: note = S or V or A
  VARIANT 51: note = I or V
  VARIANT 55: note = S or N
  VARIANT 106: note = F or Y
QVQLVQSGAEVKKPGASVKVSCKASGYTFXXYGISWVRQAPGQGLEWMGWXAPYXGNTNYAQKLQGRVTMTTDTSTSTAYMELRSLRSDDTAVYYCARDAGTYSPXGYGMDVWGQGTTVTVSS

SEQ ID NO: 764 - protein, 108 aa, synthetic construct
  VARIANT 24: note = R or Q
  VARIANT 27: note = Q or E or H
  VARIANT 30: note = S or D or N
  VARIANT 31: note = S or N
  VARIANT 50: note = K or S
  VARIANT 52: note = S or Y
  VARIANT 53: note = S or Y or N
  VARIANT 56: note = S or Y
  VARIANT 90: note = Q or L or R
  VARIANT 93: note = S or K
DIQMTQSPSTLSASVGDRVTITCXASXSIXXWLAWYQQKPGKAPKLLIYXAXXLEXGVPSRFSGSGSGTEFTLTISSLQPDDFATYYCQXFQXLPPFTFGGGTKVEIK

SEQ ID NO: 765 - protein, 119 aa, synthetic construct
  VARIANT 30: note = H or R
  VARIANT 32: note = R or Y
QVQLVESGGGVVQPGRSLRLSCAASGFTFXSXGMHWVRQAPGKGLEWVAVITYDGINKYYADSVEGRFTISRDNSKNTLYLQMNSLRAEDTAVYYCARDGVYYGVYDYWGQGTLVTVSS

SEQ ID NO: 766 - protein, 113 aa, synthetic construct; identical to SEQ ID NO: 342

SEQ ID NO: 767 - protein, 124 aa, synthetic construct; identical to SEQ ID NO: 417

SEQ ID NO: 768 - protein, 107 aa, synthetic construct; identical to SEQ ID NO: 418

SEQ ID NO: 769 - protein, 125 aa, synthetic construct
  VARIANT 6: note = E or Q
  VARIANT 7: note = S or W
  VARIANT 9: note = P or A
  VARIANT 12: note = V or L
  VARIANT 16: note = Q or E
  VARIANT 23: note = T or A
  VARIANT 25: note = S or Y
  VARIANT 29: note = I or L
  VARIANT 33: note = Q or Y
  VARIANT 41: note = H or P
  VARIANT 53: note = Y or G
  VARIANT 54: note = Y or A
  VARIANT 77: note = N or D
  VARIANT 100: note = T or A
  VARIANT 105: note = E or G or D
QVQLQXXGXGLXKPSXTLSLTCXVXGGSXSSGXYWSWIRQXPGKGLEWIGEIXXSGSTRYNPSLKSRVTISVDTSKXQFSLKLSSVTAADTAVYYCARDXPYYYXGGYYYYMDVWGKGTTVTVSS

SEQ ID NO: 770 - protein, 108 aa, synthetic construct
  VARIANT 27: note = Q or E or D
  VARIANT 30: note = S or D
  VARIANT 33: note = Y or F
  VARIANT 53: note = S or D or F or Y
  VARIANT 54: note = S or T
  VARIANT 56: note = A or Q
  VARIANT 57: note = T or N
  VARIANT 92: note = V or A or D
EIVLTQSPGTLSLSPGERATLSCRASXSVXSSXLAWYQQKPGQAPRLLIYGAXXRXXGIPDRFSGSGSGTDFTLTISRLEPEDFAVYYCQQXGVVPYTFGGGTKVEIK

SEQ ID NO: 771 - protein, 121 aa, synthetic construct; identical to SEQ ID NO: 759

SEQ ID NO: 772 - protein, 107 aa, synthetic construct; identical to SEQ ID NO: 760

SEQ ID NO: 773 - protein, 10 aa, synthetic construct
  VARIANT 6: note = D or S
  VARIANT 10: note = A or G
GFTFSXYAMX

SEQ ID NO: 774 - protein, 17 aa, synthetic construct
  VARIANT 1: note = A or T
XISGSGGLTYYADSVKG

SEQ ID NO: 775 - protein, 9 aa, synthetic construct
APYGYYMDV

SEQ ID NO: 776 - protein, 11 aa, synthetic construct
RASQSISSWLA

SEQ ID NO: 777 - protein, 7 aa, synthetic construct
KASSLES

SEQ ID NO: 778 - protein, 8 aa, synthetic construct
QQYKSYIT

SEQ ID NO: 779 - protein, 10 aa, synthetic construct
  VARIANT 5: note = D or R
  VARIANT 6: note = S or V
GYTFXXYGIS

SEQ ID NO: 780 - protein, 17 aa, synthetic construct
  VARIANT 2: note = I or V
  VARIANT 6: note = S or N
WXAPYXGNTNYAQKLQG

SEQ ID NO: 781 - protein, 14 aa, synthetic construct
  VARIANT 8: note = F or Y
DAGTYSPXGYGMDV

SEQ ID NO: 782 - protein, 11 aa, synthetic construct
  VARIANT 1: note = R or Q
  VARIANT 4: note = Q or E or H
  VARIANT 7: note = S or D or N
  VARIANT 8: note = S or N
XASXSIXXWLA

SEQ ID NO: 783 - skipped

SEQ ID NO: 784 - protein, 10 aa, synthetic construct
  VARIANT 2: note = Q or L or R
  VARIANT 5: note = S or K
QXFQXLPPFT

SEQ ID NO: 785 - protein, 10 aa, synthetic construct
  VARIANT 5: note = H or R
  VARIANT 7: note = R or Y
GFTFXSXGMH

SEQ ID NO: 786 - protein, 17 aa, synthetic construct
VITYDGINKYYADSVEG

SEQ ID NO: 787 - protein, 10 aa, synthetic construct
DGVYYGVYDY

SEQ ID NO: 788 - protein, 17 aa, synthetic construct
KSSQSVLFSSNNKNYLA

SEQ ID NO: 789 - protein, 7 aa, synthetic construct
WASTRES

SEQ ID NO: 790 - protein, 9 aa, synthetic construct
QQFHSYPLT

SEQ ID NO: 791 - protein, 10 aa, synthetic construct
GGTFSSNAIG

SEQ ID NO: 792 - protein, 17 aa, synthetic construct
SIIPIIGFANYAQKFQG

SEQ ID NO: 793 - protein, 15 aa, synthetic construct
DSGYYYGASSFGMDV

SEQ ID NO: 794 - protein, 11 aa, synthetic construct
RASQSVSSNLA

SEQ ID NO: 795 - protein, 7 aa, synthetic construct
GASTRAT

SEQ ID NO: 796 - protein, 9 aa, synthetic construct
EQYNNLPLT

SEQ ID NO: 797 - protein, 11 aa, synthetic construct
  VARIANT 4: note = I or L
  VARIANT 8: note = Q or Y
GGSXSSGXYWS

SEQ ID NO: 798 - protein, 16 aa, synthetic construct
  VARIANT 3: note = Y or G
  VARIANT 4: note = Y or A
EIXXSGSTRYNPSLKS

SEQ ID NO: 799 - protein, 16 aa, synthetic construct
  VARIANT 2: note = T or A
  VARIANT 7: note = E or G or D
DXPYYYXGGYYYYMDV

SEQ ID NO: 800 - protein, 12 aa, synthetic construct
  VARIANT 4: note = Q or E or D
  VARIANT 7: note = S or D
  VARIANT 10: note = Y or F
RASXSVXSSXLA

SEQ ID NO: 801 - skipped

SEQ ID NO: 802 - protein, 9 aa, synthetic construct
  VARIANT 3: note = V or A or D
QQXGVVPYT

SEQ ID NO: 803 - protein, 10 aa, synthetic construct
GYTFANYYMH

SEQ ID NO: 804 - protein, 17 aa, synthetic construct
IINPSGGITVYAQKFQG

SEQ ID NO: 805 - protein, 12 aa, synthetic construct
GGSKVAALAFDI

SEQ ID NO: 806 - protein, 11 aa, synthetic construct
QASQDISNSLN

SEQ ID NO: 807 - protein, 7 aa, synthetic construct
DASNLET

SEQ ID NO: 808 - protein, 9 aa, synthetic construct
QQYNFHPLT

SEQ ID NO: 809 - protein, 295 aa, Homo sapiens
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKCRKAGVGQSWKENSPLNVS

SEQ ID NO: 810 - protein, 219 aa, Homo sapiens
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 811 - protein, 227 aa, synthetic construct
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRETGHHHHHH

SEQ ID NO: 812 - protein, 455 aa, synthetic construct
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRETGENLYFQGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 813 - protein, 296 aa, Macaca fascicularis
METPAWPRVPRPETAVARTLLLGWVFAQVAGASGTTNTVAAYNLTWKSTNFKTILEWEPKPINQVYTVQISTKSGDWKSKCFYTADTECDLTDEIVKDVKQTYLARVFSYPAGHVESTGSTEEPPYENSPEFTPYLETNLGQPTIQSFEQVGTKVNVTVQDEWTLVRRNDTFLSLRDVFGKDLIYTLYYWKSSSSGKKTAKTNTNEFLIDVDKGENYCFSVQAVIPSRRTANRKSTDSPVECMGHEKGESREIFYIIGAVVFVVIILVIILAISLHKCKKARVGRSWKENSPLNVA

SEQ ID NO: 814 - protein, 220 aa, Macaca fascicularis
SGTTNTVAAYNLTWKSTNFKTILEWEPKPINQVYTVQISTKSGDWKSKCFYTADTECDLTDEIVKDVKQTYLARVFSYPAGHVESTGSTEEPPYENSPEFTPYLETNLGQPTIQSFEQVGTKVNVTVQDEWTLVRRNDTFLSLRDVFGKDLIYTLYYWKSSSSGKKTAKTNTNEFLIDVDKGENYCFSVQAVIPSRRTANRKSTDSPVECMGHEKGESRE

SEQ ID NO: 815 - protein, 228 aa, synthetic construct
SGTTNTVAAYNLTWKSTNFKTILEWEPKPINQVYTVQISTKSGDWKSKCFYTADTECDLTDEIVKDVKQTYLARVFSYPAGHVESTGSTEEPPYENSPEFTPYLETNLGQPTIQSFEQVGTKVNVTVQDEWTLVRRNDTFLSLRDVFGKDLIYTLYYWKSSSSGKKTAKTNTNEFLIDVDKGENYCFSVQAVIPSRRTANRKSTDSPVECMGHEKGESRETGHHHHHH

SEQ ID NO: 816 - protein, 456 aa, synthetic construct
SGTTNTVAAYNLTWKSTNFKTILEWEPKPINQVYTVQISTKSGDWKSKCFYTADTECDLTDEIVKDVKQTYLARVFSYPAGHVESTGSTEEPPYENSPEFTPYLETNLGQPTIQSFEQVGTKVNVTVQDEWTLVRRNDTFLSLRDVFGKDLIYTLYYWKSSSSGKKTAKTNTNEFLIDVDKGENYCFSVQAVIPSRRTANRKSTDSPVECMGHEKGESRETGENLYFQGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 817 - protein, 294 aa, Mus musculus
MAILVRPRLLAALAPTFLGCLLLQVTAGAGIPEKAFNLTWISTDFKTILEWQPKPTNYTYTVQISDRSRNWKNKCFSTTDTECDLTDEIVKDVTWAYEAKVLSVPRRNSVHGDGDQLVIHGEEPPFTNAPKFLPYRDTNLGQPVIQQFEQDGRKLNVVVKDSLTLVRKNGTFLTLRQVFGKDLGYIITYRKGSSTGKKTNITNTNEFSIDVEEGVSYCFFVQAMIFSRKTNQNSPGSSTVCTEQWKSFLGETLIIVGAVVLLATIFIILLSISLCKRRKNRAGQKGKNTPSRLA

SEQ ID NO: 818 - protein, 223 aa, Mus musculus
AGIPEKAFNLTWISTDFKTILEWQPKPTNYTYTVQISDRSRNWKNKCFSTTDTECDLTDEIVKDVTWAYEAKVLSVPRRNSVHGDGDQLVIHGEEPPFTNAPKFLPYRDTNLGQPVIQQFEQDGRKLNVVVKDSLTLVRKNGTFLTLRQVFGKDLGYIITYRKGSSTGKKTNITNTNEFSIDVEEGVSYCFFVQAMIFSRKTNQNSPGSSTVCTEQWKSFLGE

SEQ ID NO: 819 - protein, 231 aa, synthetic construct
AGIPEKAFNLTWISTDFKTILEWQPKPTNYTYTVQISDRSRNWKNKCFSTTDTECDLTDEIVKDVTWAYEAKVLSVPRRNSVHGDGDQLVIHGEEPPFTNAPKFLPYRDTNLGQPVIQQFEQDGRKLNVVVKDSLTLVRKNGTFLTLRQVFGKDLGYIITYRKGSSTGKKTNITNTNEFSIDVEEGVSYCFFVQAMIFSRKTNQNSPGSSTVCTEQWKSFLGETGHHHHHH

SEQ ID NO: 820 - protein, 459 aa, synthetic construct
AGIPEKAFNLTWISTDFKTILEWQPKPTNYTYTVQISDRSRNWKNKCFSTTDTECDLTDEIVKDVTWAYEAKVLSVPRRNSVHGDGDQLVIHGEEPPFTNAPKFLPYRDTNLGQPVIQQFEQDGRKLNVVVKDSLTLVRKNGTFLTLRQVFGKDLGYIITYRKGSSTGKKTNITNTNEFSIDVEEGVSYCFFVQAMIFSRKTNQNSPGSSTVCTEQWKSFLGETGENLYFQGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 821 - protein, 120 aa, synthetic construct
EVQLVQSGAEVKKPGESLRISCKGSGYTFAPYWIEWVRQMPGKGLEWMGDILPGTGFTTYSPSFQGHVTISADKSISTAYLQWSSLKASDTAMYYCARSGYYGNSGFAYWGQGTLVTVSS

SEQ ID NO: 822 - protein, 113 aa, synthetic construct
DIVMTQTPLSLPVTPGEPASISCKSSQSLLSSGNQKNYLTWYLQKPGQSPQLLIYWASTRESGVPDRFSGSGSGTDFTLKISRVEAEDVGVYYCQNDYTYPLTFGQGTKLEIK

SEQ ID NO: 823 - protein, 5 aa, synthetic construct
GGGGS

SEQ ID NO: 824 - protein, 292 aa, Sus scrofa
MATPTGPPVSCPKAAVARALLLGWVLVQVAGATGTTDVIVAYNLTWKSTNFKTILEWEPKPINYVYTVQISPRLGDWKNKCFHTTDTECDVTDEIMRNVKETYVARVLSYPADTVLTAQEPPFTNSPPFTPYLDTNLGQPVIQSFEQVGTKLNVTVEAARTLVRVNGTFLRLRDVFGKDLNYTLYYWRASSTGKKKATTNTNEFLIDVDKGENYCFSVQAVIPSRRVNQKSPESRIECTSQEKAVSRELFLIVGAVVFAVIVFVLVLSVSLYKCRKERAGPSGKENAPLNVA

SEQ ID NO: 825 - protein, 216 aa, Sus scrofa
TGTTDVIVAYNLTWKSTNFKTILEWEPKPINYVYTVQISPRLGDWKNKCFHTTDTECDVTDEIMRNVKETYVARVLSYPADTVLTAQEPPFTNSPPFTPYLDTNLGQPVIQSFEQVGTKLNVTVEAARTLVRVNGTFLRLRDVFGKDLNYTLYYWRASSTGKKKATTNTNEFLIDVDKGENYCFSVQAVIPSRRVNQKSPESRIECTSQEKAVSRE

SEQ ID NO: 826 - protein, 224 aa, synthetic construct
TGTTDVIVAYNLTWKSTNFKTILEWEPKPINYVYTVQISPRLGDWKNKCFHTTDTECDVTDEIMRNVKETYVARVLSYPADTVLTAQEPPFTNSPPFTPYLDTNLGQPVIQSFEQVGTKLNVTVEAARTLVRVNGTFLRLRDVFGKDLNYTLYYWRASSTGKKKATTNTNEFLIDVDKGENYCFSVQAVIPSRRVNQKSPESRIECTSQEKAVSRETGHHHHHH

SEQ ID NO: 827 - protein, 452 aa, synthetic construct
TGTTDVIVAYNLTWKSTNFKTILEWEPKPINYVYTVQISPRLGDWKNKCFHTTDTECDVTDEIMRNVKETYVARVLSYPADTVLTAQEPPFTNSPPFTPYLDTNLGQPVIQSFEQVGTKLNVTVEAARTLVRVNGTFLRLRDVFGKDLNYTLYYWRASSTGKKKATTNTNEFLIDVDKGENYCFSVQAVIPSRRVNQKSPESRIECTSQEKAVSRETGENLYFQGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 828 - protein, 118 aa, synthetic construct
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSS

SEQ ID NO: 829 - protein, 107 aa, synthetic construct
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIK

SEQ ID NO: 830 - protein, 117 aa, synthetic construct
QVQLVESGGGVVQPGRSLRLSCKASGFNIKDYYMHWVRQAPGKGLEWIGLIDPENGNTIYDPKFQGRFTISADNSKNTLFLQMDSLRPEDTAVYYCARDNSYYFDYWGQGTPVTVSS

SEQ ID NO: 831 - protein, 107 aa, synthetic construct
DIQMTQSPSSLSASVGDRVTITCKASQDIRKYLNWYQQKPGKAPKLLIYYATSLADGVPSRFSGSGSGTDYTFTISSLQPEDIATYYCLQHGESPYTFGQGTKLEIT

SEQ ID NO: 832 - protein, 292 aa, Oryctolagus cuniculus
MAPPTRLQVPRPGTAVPYTVLLGWLLAQVARAADTTGRAYNLTWKSTNFKTILEWEPKSIDHVYTVQISTRLENWKSKCFLTAETECDLTDEVVKDVGQTYMARVLSYPARNGNTTGFPEEPPFRNSPEFTPYLDTNLGQPTIQSFEQVGTKLNVTVQDARTLVRRNGTFLSLRAVFGKDLNYTLYYWRASSTGKKTATTNTNEFLIDVDKGENYCFSVQAVIPSRKRKQRSPESLTECTSREQGRAREMFFIIGAVVVVALLIIVLSVTVYKCRKARAGPSGKESSPLNIA

SEQ ID NO: 833 - protein, 218 aa, Oryctolagus cuniculus
ADTTGRAYNLTWKSTNFKTILEWEPKSIDHVYTVQISTRLENWKSKCFLTAETECDLTDEVVKDVGQTYMARVLSYPARNGNTTGFPEEPPFRNSPEFTPYLDTNLGQPTIQSFEQVGTKLNVTVQDARTLVRRNGTFLSLRAVFGKDLNYTLYYWRASSTGKKTATTNTNEFLIDVDKGENYCFSVQAVIPSRKRKQRSPESLTECTSREQGRAREM

SEQ ID NO: 834 - protein, 226 aa, synthetic construct
ADTTGRAYNLTWKSTNFKTILEWEPKSIDHVYTVQISTRLENWKSKCFLTAETECDLTDEVVKDVGQTYMARVLSYPARNGNTTGFPEEPPFRNSPEFTPYLDTNLGQPTIQSFEQVGTKLNVTVQDARTLVRRNGTFLSLRAVFGKDLNYTLYYWRASSTGKKTATTNTNEFLIDVDKGENYCFSVQAVIPSRKRKQRSPESLTECTSREQGRAREMTGHHHHHH

SEQ ID NO: 835 - protein, 452 aa, synthetic construct
ADTTGRAYNLTWKSTNFKTILEWEPKSIDHVYTVQISTRLENWKSKCFLTAETECDLTDEVVKDVGQTYMARVLSYPARNGNTTGFPEEPPFRNSPEFTPYLDTNLGQPTIQSFEQVGTKLNVTVQDARTLVRRNGTFLSLRAVFGKDLNYTLYYWRASSTGKKTATTNTNEFLIDVDKGENYCFSVQAVIPSRKRKQRSPESLTECTSREQGRAREMENLYFQGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 836 - protein, 123 aa, synthetic construct
QVQLVQSGAEVKKPGASVKVSCKASGYTFDAYGISWVRQAPGQGLEWMGWIAPYSGNTNYAQKLQGRVTMTTDTSTSTAYMELRSLRSDDTAVYYCARDAGTYSPFGYGMDVWGQGTTVTVSS

SEQ ID NO: 837 - protein, 108 aa, synthetic construct; identical to SEQ ID NO: 190

SEQ ID NO: 838 - protein, 224 aa, Rattus norvegicus
AGTPPGKAFNLTWISTDFKTILEWQPKPTNYTYTVQISDRSRNWKYKCTGTTDTECDLTDEIVKDVNWTYEARVLSVPWRNSTHGKETLFGTHGEEPPFTNARKFLPYRDTKIGQPVIQKYEQGGTKLKVTVKDSFTLVRKNGTFLTLRQVFGNDLGYILTYRKDSSTGRKTNTTHTNEFLIDVEKGVSYCFFAQAVIFSRKTNHKSPESITKCTEQWKSVLGE

SEQ ID NO: 839 - protein, 224 aa, synthetic construct
AGTPPGKAFNLTWISTDFKTILEWQPKPTNYTYTVQISDRSRNWKYKCTGTTDTECDLTDEIVKDVNWTYEARVLSVPWRNSTHGKETLFGTHGEEPPFTNARKFLPYRDTKLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 840 - protein, 218 aa, synthetic construct
AGTPPGKAFNLTWISTDFKTILEWQPKPTNYTYTVQISDRSRNWKYKCTGTTDTECDLTDEIVKDVNWTYEARVLSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 841 - protein, 218 aa, synthetic construct
AGTPPGKAFNLTWISTDFKTILEWQPKPTNYTYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 842 - protein, 219 aa, synthetic construct
SGTTNTVAAYNLTWKSTNFKTILEWEPKPVNQVYTVQISDRSRNWKYKCTGTTDTECDLTDEIVKDVNWTYEARVLSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 843 - protein, 219 aa, synthetic construct
SGTTNTVAAYNLTWKSTNFKTILEWEPKPVNQVYTVQISTKSGDWKSKCFYTTDTECDLTDEIVKDVKQTYLARVFSVPWRNSTHGTHGEEPPFTNARKFLPYRDTKLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 844 - protein, 225 aa, synthetic construct
SGTTNTVAAYNLTWKSTNFKTILEWEPKPVNQVYTVQISTKSGDWKSKCFYTTDTECDLTDEIVKDVKQTYLARVFSVPWRNSTHGKETLFGTHGEEPPFTNARKFLPYRDTKLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 845 - protein, 225 aa, synthetic construct
SGTTNTVAAYNLTWKSTNFKTILEWEPKPVNQVYTVQISTKSGDWKSKCFYTTDTECDLTDEIVKDVKQTYLARVFSVPWRNSTHGKETLFGTHGEEPPYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 846 - protein, 219 aa, synthetic construct
SGTTNTVAAYNLTWKSTNFKTILEWEPKPVNQVYTVQISTKSGDWKSKCFYTTDTECDLTDEIVKDVKQTYLARVFSYPAGNVESTGSAGEPLFTNARKFLPYRDTKLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 847 - protein, 219 aa, synthetic construct
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNIGQPVIQKYEQGGTKLKVTVKDSFTLVRKNGTFLTLRQVFGNDLGYILTYRKDSSTGRKTNTTHTNEFLIDVEKGVSYCFFAQAVIFSRKTNHKSPESITKCTEQWKSVLGE

SEQ ID NO: 848 - protein, 219 aa, synthetic construct
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNIGQPVIQKYEQGGTKLKVTVKDSFTLVRKNGTFLTLRQVFGNDLGYILTYRKSSSSGKKTAKTNTNEFLIDVDKGENYCFSVQAVIPSRTVNRKSTDSPVECMGQEKGEFRE

SEQ ID NO: 849 - protein, 219 aa, synthetic construct
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNIGQPVIQKYEQGGTKLKVTVKDSFTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 850 - protein, 219 aa, synthetic construct
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRKNGTFLTLRQVFGNDLGYILTYRKSSSSGKKTAKTNTNEFLIDVDKGENYCFSVQAVIPSRTVNRKSTDSPVECMGQEKGEFRE

SEQ ID NO: 851 - protein, 219 aa, synthetic construct
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRKNGTFLTLRQVFGKDLIYTLYYWKSSSSGKKTAKTNTNEFLIDVDKGENYCFSVQAVIPSRTVNRKSTDSPVECMGQEKGEFRE

SEQ ID NO: 852 - protein, 219 aa, synthetic construct
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRKNGTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 853 - protein, 219 aa, synthetic construct
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLTLRQVFGKDLIYTLYYWKSSSSGKKTAKTNTNEFLIDVDKGENYCFSVQAVIPSRTVNRKSTDSPVECMGQEKGEFRE

SEQ ID NO: 854 - protein, 219 aa, synthetic construct
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGNDLGYILTYRKSSSSGKKTAKTNTNEFLIDVDKGENYCFSVQAVIPSRTVNRKSTDSPVECMGQEKGEFRE

SEQ ID NO: 855 - protein, 219 aa, synthetic construct
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGNDLIYTLYYWKSSSSGKKTAKTNTNEFLIDVDKGENYCFSVQAVIPSRTVNRKSTDSPVECMGQEKGEFRE

SEQ ID NO: 856 - protein, 219 aa, synthetic construct
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLGYILTYRKSSSSGKKTAKTNTNEFLIDVDKGENYCFSVQAVIPSRTVNRKSTDSPVECMGQEKGEFRE

SEQ ID NO: 857 - protein, 219 aa, synthetic construct
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKDSSTGRKTNTTHTNEFLIDVEKGVSYCFFAQAVIFSRKTNHKSPESITKCTEQWKSVLGE

SEQ ID NO: 858 - protein, 219 aa, synthetic construct
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKDSSTGRKTNTTHTNEFLIDVEKGVSYCFFAQAVIPSRTVNRKSTDSPVECMGQEKGEFRE

SEQ ID NO: 859 - protein, 219 aa, synthetic construct
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKDSSTGRKTNTTHTNEFLIDVDKGENYCFSVQAVIPSRTVNRKSTDSPVECMGQEKGEFRE

SEQ ID NO: 860 - protein, 219 aa, synthetic construct
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKDSSTGRKTAKTNTNEFLIDVDKGENYCFSVQAVIPSRTVNRKSTDSPVECMGQEKGEFRE

SEQ ID NO: 861 - protein, 219 aa, synthetic construct
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTNTTHTNEFLIDVDKGENYCFSVQAVIPSRTVNRKSTDSPVECMGQEKGEFRE

SEQ ID NO: 862 - protein, 219 aa, synthetic construct
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVEKGVSYCFFAQAVIPSRTVNRKSTDSPVECMGQEKGEFRE

SEQ ID NO: 863 - protein, 219 aa, synthetic construct
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIFSRKTNHKSPESITKCTEQWKSVLGE

SEQ ID NO: 864 - protein, 224 aa, synthetic construct
AGTPPGKAFNLTWISTDFKTILEWQPKPTNYTYTVQISDRSRNWKYKCTGTTDTECDLTDEIVKDVNWTYEARVLSVPWRNSTHGKETLFGTHGEEPPFTNARKFLPYRDTKIGQPVIQKYEQGGTKLKVTVKDSFTLVRRNNTFLSLRDVFGKDLIYTLYYWKSSSSGKKTAKTNTNEFLIDVDKGENYCFSVQAVIFSRKTNHKSPESITKCTEQWKSVLGE

SEQ ID NO: 865 - protein, 219 aa, synthetic construct
SGTTNTVAAYNLTWKSTNFKTILEWEPKPVNQVYTVQISTKSGDWKSKCFYTTDTECDLTDEIVKDVNQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 866 - protein, 219 aa, synthetic construct; identical to SEQ ID NO: 855

SEQ ID NO: 867 - protein, 219 aa, synthetic construct
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTHTNEFLIDVDKGENYCFSVQAVIPSRKVNRKSTDSPVECMGQEKGEFRE

SEQ ID NO: 868 - protein, 123 aa, synthetic construct
  VARIANT 31: note = V or A
  VARIANT 55: note = N or S
QVQLVQSGAEVKKPGASVKVSCKASGYTFDXYGISWVRQAPGQGLEWMGWIAPYXGNTNYAQKLQGRVTMTTDTSTSTAYMELRSLRSDDTAVYYCARDAGTYSPFGYGMDVWGQGTTVTVSS

SEQ ID NO: 869 - protein, 108 aa, synthetic construct
  VARIANT 24: note = R or Q
  VARIANT 27: note = Q or E
  VARIANT 30: note = S or N
  VARIANT 31: note = S or N
  VARIANT 52: note = S or Y
  VARIANT 53: note = S or N
  VARIANT 56: note = S or Y
  VARIANT 90: note = Q or L
  VARIANT 93: note = S or K
DIQMTQSPSTLSASVGDRVTITCXASXSIXXWLAWYQQKPGKAPKLLIYKAXXLEXGVPSRFSGSGSGTEFTLTISSLQPDDFATYYCQXFQXLPPFTFGGGTKVEIK

SEQ ID NO: 870 - protein, 123 aa, synthetic construct
  VARIANT 55: note = N or S
QVQLVQSGAEVKKPGASVKVSCKASGYTFRSYGISWVRQAPGQGLEWMGWVAPYXGNTNYAQKLQGRVTMTTDTSTSTAYMELRSLRSDDTAVYYCARDAGTYSPYGYGMDVWGQGTTVTVSS

SEQ ID NO: 871 - protein, 108 aa, synthetic construct
  VARIANT 24: note = Q or R
  VARIANT 27: note = Q or H
  VARIANT 30: note = S or D
  VARIANT 50: note = K or S
  VARIANT 53: note = S or Y
  VARIANT 90: note = Q or L or R
DIQMTQSPSTLSASVGDRVTITCXASXSIXSWLAWYQQKPGKAPKLLIYXASXLESGVPSRFSGSGSGTEFTLTISSLQPDDFATYYCQXFQSLPPFTFGGGTKVEIK

SEQ ID NO: 872 - protein, 10 aa, synthetic construct
  VARIANT 6: note = V or A
GYTFDXYGIS

SEQ ID NO: 873 - protein, 17 aa, synthetic construct
  VARIANT 6: note = N or S
WIAPYXGNTNYAQKLQG

SEQ ID NO: 874 - protein, 14 aa, synthetic construct
DAGTYSPFGYGMDV

SEQ ID NO: 875 - protein, 11 aa, synthetic construct
  VARIANT 1: note = R or Q
  VARIANT 4: note = Q or E
  VARIANT 7: note = S or N
  VARIANT 8: note = S or N
XASXSIXXWLA

SEQ ID NO: 876 - protein, 7 aa, synthetic construct
  VARIANT 3: note = S or Y
  VARIANT 4: note = S or N
  VARIANT 7: note = S or Y
KAXXLEX

SEQ ID NO: 877 - protein, 10 aa, synthetic construct
  VARIANT 2: note = Q or L
  VARIANT 5: note = S or K
QXFQXLPPFT

SEQ ID NO: 878 - protein, 10 aa, synthetic construct
GYTFRSYGIS

SEQ ID NO: 879 - protein, 17 aa, synthetic construct
  VARIANT 6: note = N or S
WVAPYXGNTNYAQKLQG

SEQ ID NO: 880 - protein, 14 aa, synthetic construct
DAGTYSPYGYGMDV

SEQ ID NO: 881 - protein, 11 aa, synthetic construct
  VARIANT 1: note = R or Q
  VARIANT 4: note = Q or H
  VARIANT 7: note = S or D
XASXSIXSWLA

SEQ ID NO: 882 - protein, 7 aa, synthetic construct
  VARIANT 1: note = K or S
  VARIANT 4: note = S or Y
XASXLES

SEQ ID NO: 883 - protein, 10 aa, synthetic construct
  VARIANT 2: note = Q or L or R
QXFQSLPPFT

SEQ ID NO: 884 - protein, 10 aa, synthetic construct
GYTFDAYGIS

SEQ ID NO: 885 - protein, 17 aa, synthetic construct
WIAPYSGNTNYAQKLQG

SEQ ID NO: 886 - protein, 14 aa, synthetic construct
DAGTYSPFGYGMDV

SEQ ID NO: 887 - protein, 11 aa, synthetic construct
RASESISNWLA

SEQ ID NO: 888 - protein, 7 aa, synthetic construct
KAYSLEY

SEQ ID NO: 889 - protein, 10 aa, synthetic construct
QQFQKLPPFT

SEQ ID NO: 890 - protein, 5 aa, synthetic construct
AYGIS

SEQ ID NO: 891 - protein, 17 aa, synthetic construct
WIAPYSGNTNYAQKLQG

SEQ ID NO: 892 - protein, 14 aa, synthetic construct
DAGTYSPFGYGMDV

SEQ ID NO: 893 - protein, 11 aa, synthetic construct
RASESISNWLA

SEQ ID NO: 894 - protein, 7 aa, synthetic construct
KAYSLEY

SEQ ID NO: 895 - protein, 10 aa, synthetic construct
QQFQKLPPFT

SEQ ID NO: 896 - protein, 7 aa, synthetic construct
GYTFDAY

SEQ ID NO: 897 - protein, 4 aa, synthetic construct
PYSG

SEQ ID NO: 898 - protein, 12 aa, synthetic construct
AGTYSPFGYGMD

SEQ ID NO: 899 - protein, 7 aa, synthetic construct
SESISNW

SEQ ID NO: 900 - skipped

SEQ ID NO: 901 - protein, 7 aa, synthetic construct
FQKLPPF

SEQ ID NO: 902 - protein, 10 aa, synthetic construct
GYTFDAYGIS

SEQ ID NO: 903 - protein, 10 aa, synthetic construct
WIAPYSGNTN

SEQ ID NO: 904 - protein, 14 aa, synthetic construct
DAGTYSPFGYGMDV

SEQ ID NO: 905 - protein, 11 aa, synthetic construct
RASESISNWLA

SEQ ID NO: 906 - protein, 7 aa, synthetic construct
KAYSLEY

SEQ ID NO: 907 - protein, 10 aa, synthetic construct
QQFQKLPPFT

SEQ ID NO: 908 - protein, 6 aa, synthetic construct
DAYGIS

SEQ ID NO: 909 - protein, 13 aa, synthetic construct
WMGWIAPYSGNTN

SEQ ID NO: 910 - protein, 15 aa, synthetic construct
ARDAGTYSPFGYGMD

SEQ ID NO: 911 - protein, 7 aa, synthetic construct
SNWLAWY

SEQ ID NO: 912 - protein, 10 aa, synthetic construct
LLIYKAYSLE

SEQ ID NO: 913 - protein, 9 aa, synthetic construct
QQFQKLPPF

SEQ ID NO: 914 - protein, 8 aa, synthetic construct
GYTFDAYG

SEQ ID NO: 915 - protein, 8 aa, synthetic construct
IAPYSGNT

SEQ ID NO: 916 - protein, 16 aa, synthetic construct
ARDAGTYSPFGTGMDV

SEQ ID NO: 917 - protein, 6 aa, synthetic construct
ESISNW

SEQ ID NO: 918 - skipped

SEQ ID NO: 919 - protein, 10 aa, synthetic construct
QQFQKLPPFT

SEQ ID NO: 920 - protein, 4 aa, Homo sapiens
ELLG

SEQ ID NO: 921 - protein, 4 aa, Homo sapiens
EFLG

SEQ ID NO: 922 - protein, 6 aa, synthetic construct
  source 1..6: note = Synthetic 6xHis tag
HHHHHH